(19) United States
(12) Patent Application Publication
(10) Pub. No.: US 2026/0028391 A1
(43) Pub. Date: Jan. 29, 2026

(54) BROADLY NEUTRALIZING ANTIBODIES AGAINST RSV AND MPV PARAMYXOVIRUSES

(71) Applicants: Humabs BioMed SA, Bellinzona (CH); Vir Biotechnology, Inc., San Francisco, CA (US)

(72) Inventors: Elisabetta Cameroni, Claro (CH); Davide Corti, Bellinzona (CH); Luca PICCOLI, Bellinzona (CH); Gyorgy Snell, San Francisco, CA (US)

(21) Appl. No.: 18/996,878

(22) PCT Filed: Jul. 27, 2023
(86) PCT No.: PCT/US2023/071126

§ 371 Date: Jan. 17, 2025

Related U.S. Application Data
(60) Provisional application No. 63/392,834, filed on Jul. 27, 2022.
(60) Provisional application No. 63/395,269, filed on Aug. 4, 2022.
(60) Provisional application No. 63/427,391, filed on Nov. 22, 2022.
(60) Provisional application No. 63/430,310, filed on Dec. 5, 2022.
(60) Provisional application No. 63/482,538, filed on Jan. 31, 2023.
(60) Provisional application No. 63/494,751, filed on Apr. 6, 2023.

(51) Int. Cl. C07K 16/10; A61K 39/00; A61P 31/14; G01N 33/569

(52) U.S. Cl. CPC C07K 16/1027; A61P 31/14; G01N 33/56983; A61K 2039/505; C07K 2317/33; C07K 2317/732; C07K 2317/76; C07K 2317/92; C07K 2317/94; G01N 2333/13; G01N 2469/10

(57) ABSTRACT

The instant disclosure provides antibodies and antigen-binding fragments that can bind to a RSV and/or MPV fusion glycoprotein and can neutralize a RSV and/or MPV infection. Also provided are polynucleotides that encode an antibody, vectors that comprise such polynucleotides, host cells that can express the antibodies, related compositions, and methods of using the herein disclosed compositions to, for example, treat or prevent a RSV and/or MPV infection.

### REFERENCE TO AN ELECTRONIC SEQUENCE LISTING

The contents of the electronic sequence listing (430WO_SeqListing.xml; Size: 1030 kilobytes; and Date of Creation: Jul. 26, 2023) are herein incorporated by reference in their entirety.

### BACKGROUND

Respiratory Syncytial Virus (RSV) and Metapneumovirus (MPV) are common cold viruses belonging to the family of paramyxovirus that share target population and represent a major health problem in newborns and immunocompromised patients.

RSV is the major cause of acute respiratory tract disease in infants and adults across the globe. Between 0.5% and 3.2% of children with RSV infection require hospitalization (Thompson et al., JAMA: The Journal of the American Medical Association 289: 179-186 (2003)), and 5% to 10% of children have prolonged severe infection, a factor believed to be predisposing to wheezing and asthma-like symptoms later in childhood. Immunity to RSV appears to be short-lived, thus re-infections are frequent (Ogra, Paediatric Respiratory Reviews 5 Suppl A:S119-126 (2003)).

The human MPV was isolated for the first time in 2001 and is now recognized to be the second major cause of acute respiratory tract disease in infants and adults; it is estimated that it infects over 50% of infants by two years of age and almost all children by five years. MPV accounts for roughly 5 to 15% of respiratory disease in hospitalized young children (Alto, The Journal of the American Board of Family Practice/American Board of Family Practice 17:466-469 (2004); Williams et al., N Engl J Med 350:443-450 (2004)). Infection with MPV is a significant burden of disease in at-risk premature infants, chronic lung disease of prematurity, congestive heart disease, and immunodeficiency (Martino et al., Biology of Blood and Marrow Transplantation: Journal of the American Society for Blood and Marrow Transplantation 11:781-796 (2005)).

Co-infections with MPV and RSV may be common given their prevalence and overlapping winter epidemics. Although it is unclear whether synergistic pathology can occur between these two viruses, exacerbations leading to particularly severe respiratory tract disease were observed in some children co-infected with MPV and RSV (Greensill, Emerging Infectious Diseases 9:372 (2003)).

RSV, which belongs to the Pneumovirus genus of the subfamily Pneumoviriniae, and MPV, which belongs to the Metapneumovirus genus of the subfamily Pneumoviriniae, have some similarities in their genetic structure, though MPV lacks the non-structural genes NS1 and NS2 found in RSV. The RSV and MPV envelopes contain three virally encoded transmembrane surface glycoproteins: the major attachment glycoprotein G, the fusion glycoprotein F, and the small hydrophobic SH protein. Although the RSV and MPV envelopes contain proteins that are functionally similar, it is important to note, however, that the F proteins of RSV and MPV share only 33% amino acid sequence identity.

The RSV and MPV F glycoproteins direct viral penetration by fusion between the virion envelope and the host cell plasma membrane. Later in infection, F protein expressed on the cell surface can mediate fusion with neighboring cells to form syncytia (Collins et al., PNAS 81:7683-7687 (1984)). In both cases, the N-terminus of the F subunit that is created by proteolytic cleavage and contains hydrophobic stretch of amino acids, called the fusion peptide, inserts directly into the target membrane to initiate fusion. After binding to the target cell and subsequent activation, the metastable pre-fusion F protein undergoes a series of structural rearrangements that result in the insertion of the fusion peptide into the target cell membrane, followed by the formation of a stable helical bundle that forms as the viral and cell membranes are opposed. These structural changes lead to the formation of a stable post-fusion F protein.

Only one vaccine for RSV has been approved in the US and is available only for older patients and no vaccine is available for MPV. As the RSV case makes clear, even as more vaccines are developed, they may not be readily available for all age groups.

Evidence for the role of serum antibodies in protection against RSV virus has emerged from epidemiological as well as animal studies. In infants, titers of maternally transmitted antibodies correlate with resistance to serious disease (Glezen et al., The Journal of Pediatrics 98:708-715 (1981)) and in adults incidence and severity of lower respiratory tract involvement is diminished in the presence of high levels of serum RSV neutralizing antibodies (Mcintosh et al., The Journal of Infectious Diseases 138:24-32 (1978)). A monoclonal antibody, palivizumab (Synagis), is registered for the prevention of RSV infection in premature newborns, palivizumab, however, is not always effective in preventing RSV infection and is not effective therapeutically. Further, prolonged pulmonary replication of RSV in the presence of palivizumab is followed in animals by the appearance of resistant virus strains (Zhao and Sullender, Journal of Virology 79:3962-3968 (2005)). Another monoclonal antibody. nirsevimab (Astra Zeneca/Sanofi), was recently approved in Europe and is still in the approval process for RSV infections in infants and very young children, and clesrovimab (Merck) is in clinical studies. Longer-term studies for this antibody are not available. Currently there are no monoclonal antibodies for the treatment or prevention of MPV infection.

The lack of a good working animal model for the most severe forms of RSV infection is related to the fact that RSV and MPV are host-restricted Pneumovirus pathogens. The development of new drugs for the therapy of RSV and MPV infections has been hampered by the lack of an animal model able to recapitulate all the symptoms and severity of the human disease. Indeed, RSV and MPV are not natural mouse pathogens and induce only a limited, minimally symptomatic, and rapidly aborted primary infection in response to a massive, non-physiologic inoculum of the virus. Pneumonia virus of mice (PVM) is a natural rodent Pneumovirus pathogen which belongs to the same family, subfamily and genus (Pneumovirus) of human and bovine RSV.

The PVM F protein shares only 40% amino acid identity with huma RSV F protein, but has the same genetic organization with the exception of the M2-L overlap which is present in RSV but absent in PVM. The infection by the natural mouse pathogen PVM replicates many of the signs and symptoms of the most severe forms of RSV as it occurs in human infants. PVM infection is characterized by rapid virus replication accompanied by a massive inflammatory response that leads to respiratory failure and death (Rosemberg and Domachowske, Immunology Letter 118:6-12 (2008)). PVM infection in mice is therefore considered to be the most relevant animal model of RSV and MPV severe infections of humans. The lack of preventive treatment for MPV infection and of widely available vaccines against RSV and MPV infections, as well as the therapeutic inefficacy of palivizumab and lack of longer-term data for nirsevimab and clesrovimab, highlight the need for new preventive and therapeutic agents against these prominent human pathogens.

### BRIEF DESCRIPTION OF THE DRAWINGS

FIG. 1 as described in Example 1 shows the results of a neutralization assay against RSV infection in HEp-2 cells.

FIG. 2 as described in Example 3 shows the results of a neutralization assay against MPV infection in HEp-2 cells (reported as IC50 in μg/ml). Also tested was antibody MPH12, along with antibodies MPE33, MPE8, MPF5, and RSD5.

FIGS. 3A-3U as described in Example 6 show binding of antibodies to RSV-F, MPV-F D280 (“MPV-F” in the figures), and MPV-F N280 proteins (also referred to as “MPV-F D280N”), as measured by surface plasmon resonance (SPR). Sample ID at the top of each graph in FIGS. 3A-3R (see also FIG. 3V) correlates with MPK antibody name (e.g. sample 9 is MPK9, sample 44 is MPK44); FIGS. 3S-3U show data for antibodies MPH12, MPE8, or RSD5, as indicated by the Sample ID at the top of each figure.

FIGS. 4A-4I as described in Example 6 show individual SPR data plots for each antibody pair tested in the competitive binding assay.

FIG. 5A, as described in Example 6, shows the results of a post-fusion RSV F protein-binding assay using MPE8.

FIG. 5B, as described in Example 6, shows the results of a post-fusion RSV F protein-binding assay using MPH12.

FIGS. 6A and 6B as described in Example 7 show neutralization by MPE8 and MPH12 antibodies against RSV A and B strains, and MPV A and B strains (reported as ng/ml). FIG. 6A shows neutralization of RSV strains. FIG. 6B shows neutralization of MPV A and B strains.

FIG. 7 as described in Example 8 shows binding of MPH12-v1, MPH12-v2, MPH12-v3, and MPH12-v4 to DS-Cav1 as measured by ELISA (reported in ng/ml). MPH12 was tested as a reference.

FIGS. 8A and 8B as described in Example 8 show neutralization of RSV A/A2/61 (FIG. 8A) and MPV A1/6621 (FIG. 8B) as measured by ELISA (reported in ng/ml) by antibodies MPH12v1, MPH12v2, MPH12v3, and MPH12v4. MPH12 (“MPH12-rIgG1”), without mutations, was tested as a reference.

FIGS. 9A and 9B as described in Example 9 show two rotated views of MPH12 H-CDR3 models generated using homology modeling. Five homology models are shown in various shades of green and the crystal structure of MPH12 is shown in grey.

FIGS. 10A-10D as described in Example 9 show four rotated views of the Fab structure of MPH12. In FIG. 10C, a groove corresponding to the space occupied by H-CDR3 is shown between L-CDR1 and L-CDR3; the H-CDR3 is shown occupying the groove.

FIGS. 11A and 11B as described in Example 9 show two rotated views of the MPH12 H-CDR3 conformation at temperatures of 100 K (FIG. 11A) and 293 K (room temperature—FIG. 11B).

FIGS. 12A and 12B as described in Example 9 show binding of MPH12 to purified RSV-F (FIG. 12A) and purified MPV-F D280 (labeled “MPV-F” in the figure) (FIG. 12B) as measured by SPR.

FIG. 13 as described in Example 10 shows the Fab structure of MPH12 obtained using 2.5A room temp X-ray structure with the broadest CDR definitions (combination of all CDR definitions).

FIG. 14 as described in Example 11 shows production titers of six variant antibodies (MPH12-v16, MPH12-v17, MPH12-v28, MPH12-v29, MPH12-v34, and MPH12-v35, as set forth in Tables 2 and 20, with reference to Table 1 and the Sequence Listing) that were selected for further characterization. MPH 12*(Parental MPH12-wt) was included as a comparator.

FIGS. 15A-15C as described in Example 11 show binding of MPH12 and MPH12 variant antibodies (MPH12-v16, MPH12-v17, MPH12-v28, MPH12-v29, MPH12-v34, and MPH12-v35) to RSV-F (FIG. 15A), MPV-F D280 (labeled “MPV-F” in the figure) (FIG. 15B), and MPV-F N280 (FIG. 15C) as measured using biolayer interferometry (BLI).

FIG. 16A and FIG. 16B as described in Example 11 show neutralization of RSV (FIG. 16A) and MPV (FIG. 16B) by the six MPH12 variant antibodies as measured using a GFP-based in vitro neutralization assay.

FIG. 17 as described in Example 11 shows thermal stability of the six MPH12 variant antibodies measured using a ProteinShift assay. MPH12 parental antibody and rituximab were included as comparators.

FIG. 18 as described in Example 11 shows lack of polyreactivity of the six MPH12 variant antibodies when tested in a Eurimmune 293 slide assay. MPH12 parental antibody (“WT”) was included as a comparator.

FIGS. 19A-19C as described in Example 11 show antibody activation of FcγRIIIa (F158 allele) and FcγRIIa (H131 allele). MPH12-v16 and MPH12-v34 were tested, along with comparator antibodies, MPE8-v3 (p11), MPE8-v3 (p12), palivizumab, and MEDI8897-YTE. Activation of Jurkat-FcγRIIIa (F158 allele) using RSV-F-transfected Expi293 target cells (FIG. 19A), Jurkat-FcγRIIIa (F158 allele) using MPV-F D280-transfected Expi293 target cells (FIG. 19B), and Jurkat-FcγRIIa (H131 allele) by RSV-F-transfected Expi293 target cells (FIG. 19C) was tested.

FIG. 20, as described in Example 12, shows the results of a competition/binning assay for MPK73, MPK65, MPK 44, MPK36, MPK15, and MPH12 with comparator antibodies MPE8, D25 and RSD5 (as set forth in Tables 2, 3 and 4, with reference to Table 1 and the Sequence Listing).

FIG. 21, as described in Example 13, shows the results of a RSV ADCC assay for MPK44 and MPK65-v2 (as set forth in Table 2, with reference to Table 1 and the Sequence Listing).

FIG. 22, as described in Example 13, shows the results of a MPV ADCC assay for MPK15 (as set forth in Table 2, with reference to Table 1 and the Sequence Listing).

FIG. 23A, as described in Example 13, shows the results of a RSV ADCC assay for various monoclonal antibodies (mAbs).

FIG. 23B, as described In Example 13, shows the results of a MPV ADCC assay for various mAbs.

FIG. 24, as described in Example 14, shows the results of a RSV-based ADCP assay for MPK44 and MPK65-v2.

FIG. 25, as described in Example 14, shows the results of a MPV-based ADCP assay for MPK15 and comparator antibodies MPE33 and MPF5.

FIGS. 26A-26D, as described in Example 14, show i) the results of RSV-based ADCP assays (FIG. 26A and FIG. 26B), and ii) the results of a MPV-based ADCP assay (FIG. 26C and FIG. 26D) for various antibodies.

FIG. 27 as described in Example 15, shows the results of a RSV escape mutants assay for various mAbs.

FIG. 28, as described in Example 16, shows the results of a MPV escape mutants assay for various mAbs.

FIG. 29, as described in Example 17, shows the results of MPK190 and MPK77 and comparator antibody nirsevimab on weight loss in RSV-infected mice.

FIG. 30, as described in Example 17, shows the results of MPK190 and MPK77 and comparator antibody nirsevimab on survival in RSV-infected mice.

FIG. 31, as described in Example 17, shows the results of MPK51 and MPK190 and comparator antibody nirsevimab on weight loss and survival in RSV-infected mice.

FIG. 32, as described in Example 17, shows the results of low doses of MPK190 and comparator antibody nirsevimab on weight loss and survival in RSV-infected mice.

FIG. 33, as described in Example 18, shows a heat map of variant antibodies binding to RSV A and RSV B strains as assessed by fluorescence-activated cell sorting (FACS).

FIG. 34, as described in Example 19, shows a heat map of variant antibodies binding to MPV strains as assessed by FACS.

FIG. 35, as described in Example 21, shows the results of MPK190 incubated with RSV A-GFP added to HEp-2 and LLC-MK2 cells after 8 cycles of reinfection.

FIG. 36, as described in Example 21, shows the results of MPK104 incubated with RSV A-GFP added to HEp-2 and LLC-MK2 cells after 8 cycles of reinfection.

FIG. 37, as described in Example 21, shows the results of MPK51 incubated with RSV A-GFP added to HEp-2 and LLC-MK2 cells after 8 cycles of reinfection.

FIG. 38, as described in Example 21, shows the results of MPK77 incubated with RSV A-GFP added to HEp-2 and LLC-MK2 cells after 8 cycles of reinfection.

FIG. 39, as described in Example 21, shows the results of palivizumab incubated with RSV A-GFP added to HEp-2 and LLC-MK2 cells after 8 cycles of reinfection.

FIG. 40, as described in Example 21, shows the results of MPK190 incubated with MPV A2-GFP added to HEp-2 and LLC-MK2 cells after 8 cycles of reinfection.

FIG. 41, as described in Example 21, shows the results of MPK104 incubated with MPV A2-GFP added to HEp-2 and LLC-MK2 cells after 8 cycles of reinfection.

FIG. 42, as described in Example 21, shows the results of MPK51 incubated with MPV A2-GFP added to HEp-2 and LLC-MK2 cells after 8 cycles of reinfection.

FIG. 43, as described in Example 21, shows the results of MPK77 incubated with MPV A2-GFP added to HEp-2 and LLC-MK2 cells after 8 cycles of reinfection.

FIG. 44, as described in Example 21, shows the results of MPE8 incubated with MPV A2-GFP added to HEp-2 and LLC-MK2 cells after 8 cycles of reinfection.

FIG. 45, as described in Example 22, shows a graph of mAb concentration for MPK190, MPK51, MPK104, and MPK77 versus number of days in an in vivo PK study.

FIG. 46, as described in Example 17, shows IC50 graphs of neutralization of mouse adapted RSV clone by MPK190, MPK104, MPK51, MPK77, and MEDI8897 in RSV-infected mice.

FIG. 47, as described in Example 17, shows the results of MPK104 and MPK51 and comparator antibody nirsevimab on weight loss in RSV-infected mice.

FIG. 48, as described in Example 17, shows the results of MPK104 and MPK51 and comparator antibody nirsevimab on survival in RSV-infected mice.

FIG. 49, as described in Example 23, shows lack of polyreactivity of selected MPK variant antibodies when tested in a Euroimmun 1522-2010 slide assay. MPK wild type antibodies (“WT”) were included as a comparator.

FIGS. 50A-50C, as described in Example 23, show binding of MPK104 variant antibodies (MPK104-v1.1, MPK104-v1.2, MPK104-v1.3, MPK104-v4.1, MPK104-v4.2, and MPK104-v4.3) to RSV-F (FIG. 50A), MPV-F (FIG. 50B), and MPV-F D280N (FIG. 50C), as measured using BLI.

FIGS. 51A-51C, as described in Example 23, show binding of MPK190 variant antibodies (MPK190-v1.1, MPK190-v1.3, MPK190-v3.1, MPK190-v3.3, MPK190-v4.1, and MPK190-v4.3) to RSV-F (FIG. 51A), MPV-F (FIG. 51B), and MPV-F D280N (FIG. 51C), as measured using BLI.

FIGS. 52A-52C, as described in Example 23, show binding of MPK51 variant antibodies (MPK51-v1.1, MPK51-v3.1, and MPK51-v4.1) to RSV-F (FIG. 52A), MPV-F (FIG. 52B), and MPV-F D280N (FIG. 52C), as measured using BLI.

FIGS. 53A-53C, as described in Example 23, show binding of MPK77 variant antibodies (MPK77-v3.1 and MPK-v1.1) to RSV-F (FIG. 53A), MPV-F (FIG. 53B), and MPV-F D280N (FIG. 53C), as measured using BLI.

FIGS. 54A-54C, as described in Example 24, show binding of MPK190 variants after forced deamidation to RSV-F (FIG. 54A), MPV-F (FIG. 54B), and MPV-F D280N (FIG. 54C), as measured using BLI.

FIG. 55, as described in Example 24, shows binding of MPK190 variants to MPV-F D280N after forced deamidation of MPK-190, as measured using BLI.

FIG. 56, as described in Example 26, shows lack of polyreactivity of selected MPK variant antibodies when tested in a Euroimmun 1522-2010 slide assay.

FIGS. 57A-57D, as described in Example 28, show results of binding (FACS) of MPK antibody variants to RSV F TM WT (FIG. 57A), MPV F D280N (FIG. 57B), MPV F (FIG. 57C), and mock binding (FIG. 57D).

FIGS. 58A-58C, as described in Example 28, shows results of a PK study on MPK190-v1.1 tested for binding to RSV F (FIG. 58A), MPV F (FIG. 58B), and D280N F (FIG. 58C) on transfected cells.

FIGS. 59A and 59B, as described in Example 29, show MPK190-v1.3 (a MPK190 variant with the NG motif) in activation of FcγRIIa (ADCP) and FcRγIIIa (ADCC), and in inducing NK cell killing (ADCC), compared to nirsevimab, in RSV A-infected Hep2 cells (FIG. 59A) and in MPV A1-infected Hep2 cells (FIG. 57B) (NK-mediated cell killing not shown in FIG. 59B).

FIGS. 60A and 60B, as described in Example 30, show the results of MPK190-v1.3, MEDI18897, MPE8-v3, and comparator antibody palivizumab at 2 mg/kg (FIG. 60A) and at 0.5 mg/kg (FIG. 60B) on weight loss in RSV-infected mice.

FIGS. 61A and 61B, as described in Example 30, show the results of MPK190-v1.3, MEDI18897, MPE8-v3, and comparator antibody palivizumab at 2 mg/kg (FIG. 61A) and at 0.5 mg/kg (FIG. 61B) on survival in RSV-infected mice.

FIG. 62A and FIG. 62B, as described in Examples 31 and 32, shows results of neutralization screenings of MPV/RSV antibodies, including MPK190-v1.3 (designated as MPK 190 in the figure) against a MPV A strain and a MPV B strain.

FIG. 63, as described in Example 31, shows a heat map of RSV-only antibodies and their binding to a panel of F proteins of recently circulating RSV B strains.

FIGS. 64A (IC50) and 64B (IC90), as described in Examples 31 and 32, show results of neutralization screenings of MPV/RSV antibodies, including MPK190-v1.3, and RSV-only antibodies, including MPK102 and MPK176, against various RSV A and RSV B lab-adapted and circulating strains.

FIG. 65, as described in Example 32 shows the results of neutralization testing of MPK190-v1.3 and various comparator antibodies against representatives of all four MPV subtypes.

FIG. 66, as described in Example 32, shows results of MPK190-v1.3 neutralization of a representative MPV B1 strain virus (NL/1/99).

FIG. 67, as described in Example 32, shows the results of ADCC testing using Hep-2 cells infected with the RSV A2 strain (MOI 2.5, NK 10:1) and MPK190.

FIGS. 68A-68E as described in Examples 31 and 32 show luminescence of FcγRIIIa (F158 allele) using RSV-FA-transfected Expi293 target cells (FIG. 68A), RSV-FB-transfected Expi293 target cells (FIG. 68B), MPV-FA-transfected Expi293 target cells (FIG. 68D), or MPV-FB-transfected Expi293 target cells (FIG. 68E), and antibody-dependent killing of RSV-A2-transfected Hep-2 cells (FIG. 68C. Legend for FIGS. 68A-68C is shown in FIG. 68C and legend for FIGS. 68D-68E is shown in FIG. 68E.

FIG. 69A and FIG. 69B, as described in Example 30, show the effects of various doses of MPK190-v1.3 and comparator antibodies on viral titers in cotton rats exposed to RSV A (FIG. 69A) or RSV B (FIG. 69B).

FIGS. 70A-F, as described in Example 32, show synergism between MPK190 and selected RSV-only antibodies for neutralization of RSV A (FIGS. 70A-C) or RSV F (FIGS. 70D-F).

FIGS. 71A and 71B, as described in Example 30, show the effects of MPK176, MPK201, and MPK65-v2, and comparator antibodies at 2 mg/kg (FIG. 71A and FIG. 71C) and at 0.5 mg/kg (FIG. 71B and FIG. 71D) on weight change and survival in RSV-infected mice.

FIG. 72, as described in Example 32, shows neutralization of various MPV subtypes by MPK190-v1.3.

FIG. 73A and FIG. 73B, as described in Example 30, show the effects of various doses of MPK190-v1.3 and comparator antibodies on viral titers in cotton rats exposed to MPV.

FIG. 74, as described in Example 32, shows predicted RSV F binding of MPK190-v1.3 to RSVF as compared to binding of comparator antibodies MPE8 and MPH12.

FIGS. 75A-75E as described in Example 33 show luminescence of FcγRIIIa (V158 allele) using RSV-FB-transfected Expi293 target cells for various combinations of MPK190-v1.2 and RSV-only antibodies.

FIG. 76, as described in Example 33 shows AUC results for the data of FIGS. 75A-75E.

FIG. 77, as described in Example 33, shows a summary graph of RSV A2 neutralization data for individual antibodies as well as combinations of RSV-only antibodies with MPK190-v1.3 from FIGS. 75A-75E.

FIG. 78, as described in Example 33, shows a summary of neutralization data for RSV A2 for combinations of RSV-only antibodies with MPK190-v1.3.

FIG. 79A and FIG. 79B, as described in Example 31 and Example 32, shows neutralization data (FIG. 79A) and a summary graphs (FIG. 79B) data for Fab fragments of RSV-only antibodies and MPK190-v1.3 for RSV A (upper graphs) and MPV (lower graphs).

FIG. 80, as described in Example 31 and Example 32, shows a summary of effector functions of MPK190-v1.3 and RSV-only antibodies.

### DETAILED DESCRIPTION

Provided herein are antibodies and antigen-binding fragments that can bind to and, in some embodiments, potently neutralize infection by RSV and/or MPV. Also provided are polynucleotides that encode the antibodies and antigen-binding fragments, vectors, host cells, and related compositions, as well as methods of using the antibodies, nucleic acids, vectors, host cells, and related compositions to treat (e.g., reduce, delay, eliminate, or prevent) a RSV and/or MPV infection in a subject and/or in the manufacture of a medicament for treating a RSV and/or MPV infection in a subject.

In some embodiments, antibodies or antigen-binding fragments thereof of the present disclosure may be able to treat infection by MPV with a D280N mutation, as well as other variants of MPV. The D280N mutation, which is found in a B2 viral subtype, has proven difficult to treat with other potential therapeutics.

In some embodiments, antibodies or antigen-binding fragments thereof of the present disclosure may bind to and/or neutralize multiple RSV and/or MPV strains (also sometimes referred to as RSV and/or MPV types or subtypes) and treat and/or prevent infection by those strains.

In other embodiments, antibodies or antigen-binding fragments thereof of the present disclosure may promote survival and decrease weight loss in RSV-infected patients at least as well as nirsevimab, an anti-RSV antibody that has had favorable clinical trial results.

In some embodiments, antibodies or antigen-binding fragments thereof of the present disclosure may provide a uniquely broad array of treatment and protection to patients, without the need for complex diagnostics to determine if an infection is RSV, MPV, or which MPV, because the antibody or antigen-binding fragment may be able to effectively bind the F protein of both RSV and MPV, even if the D280N mutation is present in MPV.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means 20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

“Nucleic acid molecule” or “polynucleotide” or “polynucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes mRNA, microRNA, siRNA, viral genomic RNA, and synthetic RNA, and polydeoxyribonucleic acid (DNA), which includes cDNA, genomic DNA, and synthetic DNA, either of which may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, with percent sequence identify defined as set forth below.

Nucleic acid molecule variants retain the capacity to encode a binding domain thereof having a functionality described herein, such as binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized. Other examples include Clustal W, MAFFT, Clustal Omega, AlignMe, Praline, GAP, BESTFIT, Needle (EMBOSS), Stretcher (EMBOSS), GGEARCH2SEQ, Water (EMBOSS), Matcher (EMBOSS), LALIGN, and SSEARCH2SEQ. A global alignment algorithm, such as a Needleman and Wunsch algorithm, can be used to align two sequences over their entire length, maximizing the number of matches and minimizes the number of gaps. Default values can be used.

To generate similarity scores for two amino acid sequences, scoring matrices can be used that assign positive scores for some non-identical amino acids (e.g., conservative amino acid substitutions, amino acids with similar physio-chemical properties, and/or amino acids that exhibit frequent substitutions in orthologs, homologs, or paralogs). Non-limiting examples of scoring matrices include PAM30, PAM70, PAM250, BLOSUM45, BLOSUM50, BLOUM62, BLOSUM80, and BLOSUM90.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide performs one or more functions of the parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (K_{D}) constant).

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity).

As used herein, the term “engineered,” “recombinant,” or “non-natural” refers to an organism, microorganism, cell, nucleic acid molecule, or vector that includes one or more genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

In certain embodiments, a nucleic acid molecule or portion thereof native to a host cell will be considered heterologous to the host cell if it has been altered or mutated, or a nucleic acid molecule native to a host cell may be considered heterologous if it has been altered with a heterologous expression control sequence or has been altered with an endogenous expression control sequence not normally associated with the nucleic acid molecule native to a host cell. In addition, the term “heterologous” can refer to a biological activity that is different, altered, or not endogenous to a host cell. As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof Δn expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it affects the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule (or, when the context clearly indicates, a fusion protein of the present disclosure). A (polynucleotide) construct may be present in a vector (e.g., a bacterial vector, a viral vector) or may be integrated into a genome. A “vector” is a nucleic acid molecule that transports another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mátés et al., Nat. Genet. 41:753, 2009). Exemplary vectors are those that are autonomously replicating (episomal vector), deliver a polynucleotide to a cell genome (e.g., viral vector), or express nucleic acid molecules to which they are linked (expression vectors).

As used herein, “expression vector” or “vector” refers to a DNA construct containing a nucleic acid molecule that is operably linked to a suitable control sequence to effect the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

The term “introduced” in the context of inserting a nucleic acid molecule into a cell, means “transfection”, “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vectors” include HIV-based lentiviral vectors for gene delivery, which can be integrative or non-integrative, have relatively large packaging capacity, and can transduce a range of different cell types. Lentiviral vectors are usually generated following transient transfection of three (packaging, envelope, and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for Example 5denovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression.

Examples of such sequences used in viral vectors include internal ribosome entry sites (IRES), furin cleavage sites, viral 2A peptide, or any combination thereof.

Plasmid vectors, including DNA-based antibody or antigen-binding fragment-encoding plasmid vectors for direct administration to a subject, are described further herein.

As used herein, the term “host” refers to a cell or microorganism targeted for genetic modification with a heterologous nucleic acid molecule to produce a polypeptide of interest (e.g., an antibody of the present disclosure).

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In the context of a RSV or MPV infection, a “host” refers to a cell or a subject infected with RSV and/or MPV. “Antigen” or “Ag”, as used herein, refers to an immunogenic molecule that provokes an immune response. This immune response may involve antibody production, activation of specific immunologically-competent cells, activation of complement, antibody dependent cytotoxicity, or any combination thereof Δn antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, stool samples, cells, biological fluids, or combinations thereof Δntigens can be produced by cells that have been modified or genetically engineered to express an antigen. Antigens can also be present in a RSV and/or MPV fusion glycoprotein antigen, such as present in a virion, or expressed or presented on the surface of a cell infected by RSV and/or MPV.

The term “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three-dimensional structural characteristics, as well as specific charge characteristics. Where an antigen is or comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

Antibodies, Antigen-Binding Fragments, and Compositions In one aspect, the present disclosure provides an isolated antibody, or an antigen-binding fragment, that binds to a fusion glycoprotein from RSV and/or MPV and/or neutralizing RSV and/or MPV in a human subject. References to an antibody or antigen-binding fragment of the disclosure hat “binds to” RSV or MPV designate binding to the fusion glycoprotein of such virus. Furthermore, any antibody or antigen-binding fragment of the disclosure that “binds to” a RSV or MPV (or any further specified antigens, epitopes, or binding sites thereof) is also “capable of binding to” or “able to bind to” such RSV or MPV.

In certain embodiments, an antibody or antigen-binding fragment of the present disclosure associates with or unites with a fusion glycoprotein of RSV or MPV, or fusion glycoproteins of both RSV and MPV, while not significantly associating or uniting with any other molecules or components in a sample.

In certain embodiments, an antibody or antigen-binding fragment of the present disclosure specifically binds to a RSV fusion glycoprotein (also referred to herein as “RSV-F”) and/or a MPV fusion glycoprotein (also referred to herein as “MPV-F”). In certain embodiments, the antibody or antigen-binding fragment binds to RSV-F in a pre-fusion conformation and, in some embodiments, also in the post-fusion conformation. Unless otherwise noted herein, binding to RSV-F refers to binding to the pre-fusion conformation. In some embodiments, the MPV-F is a wild-type protein or a protein that is wild-type at D280, but contains other mutations (also referred to herein as “MPV-F D280”). However, in other embodiments, the MPV-F contains a D280N mutation (also referred to herein as “MPV-F N280” or “D280N”). In some embodiments, the MPV-F is an otherwise wild-type protein with a mutation at D280 other than N (which may, for purposes of this disclosure, be considered a MPV-F D280 if it behaves more like MPV-F with no mutation at D280, or a MPV-F D280N if it behaves more like MPV-F with the D280N mutation).

In some embodiments, a RSV/MPV cross-binding and/or neutralizing antibody or antigen-binding fragment of the present disclosure specifically binds to: i) both RSV-F and MPV-F D280, ii) both RSV-F and MPV-F N280, iii) and/or iii) RSV-F, MPV-F D280, or MPV-F N280. In some embodiments, a RSV-binding and/or neutralizing antibody or antigen-binding fragment of the present disclosure specifically binds to RSV-F. In some embodiments, a MPV-binding and/or neutralizing antibody or antigen-binding fragment of the present disclosure specifically binds to i) MPV-F N280, ii) MPV-F D280, or iii) both MPV-F N280 and MPV-F D280.

As used herein, “specifically binds” refers to an association or union of an antibody or antigen-binding fragment to an antigen with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Alternatively, affinity may be defined as an equilibrium dissociation constant (K_{d}) of a particular binding interaction with units of M (e.g., 10⁻⁵ M to 10⁻¹³ M). Antibodies may be classified as “high-affinity” antibodies or as “low-affinity” antibodies. “High-affinity” antibodies refer to those antibodies having a Kₐ of at least 10⁹ M⁻¹, at least 10¹⁰ M⁻¹, at least 10¹¹ M⁻¹, at least 10¹² M⁻¹, or at least 10¹³ M⁻¹. “Low-affinity” antibodies refer to those antibodies having a Kₐ of up to 10⁸M⁻¹, up to 10⁷ M⁻¹, up to 10⁶ M⁻¹, up to 10⁵ M⁻¹.

Alternatively, affinity may be defined as an equilibrium dissociation constant (K_{d}) of a particular binding interaction with units of M (e.g., 10⁻⁵ M to 10⁻¹³ M).

A variety of assays are known for identifying antibodies of the present disclosure that bind a particular target, as well as determining binding domain or binding protein affinities, such as Western blot, ELISA (e.g., direct, indirect, or sandwich), analytical ultracentrifugation, spectroscopy, and surface plasmon resonance (Biacore®) analysis (see, e.g., Scatchard et al., Ann. N.Y Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for assessing affinity or apparent affinity or relative affinity are also known.

In certain examples, binding can be determined by recombinantly expressing a RSV-F and/or a MPV-F antigen in a host cell (e.g., by transfection) and immunostaining the (e.g., fixed, or fixed and permeabilized) host cell with antibody and analyzing binding by flow cytometery (e.g., using a ZE5 Cell Analyzer (BioRad®) and FlowJo software (TreeStar). In some embodiments, positive binding can be defined by differential staining by antibody of RSV-F and/or MPV-F-expressing cells versus control (e.g., mock) cells.

In some embodiments an antibody or antigen-binding fragment of the present disclosure binds to RSV-F and/or MPV-F, as measured using biolayer interferometry, or by surface plasmon resonance.

In some embodiments an antibody or antigen-binding fragment of the present disclosure may be assessed for competitive binding against another antibody or antigen-binding fragment in using surface plasmon resonance.

Certain characteristics of presently disclosed antibodies or antigen-binding fragments may be described using IC50 or EC50 values. In certain embodiments, the IC50 is the concentration of a composition (e.g., antibody) that results in half-maximal inhibition of the indicated biological or biochemical function, activity, or response. In certain embodiments, the EC50 is the concentration of a composition that provides the half-maximal response in the assay. In some embodiments, e.g., for describing the ability of a presently disclosed antibody or antigen-binding fragment to neutralize infection by RSV and/or MPV, IC50 and EC50 are used interchangeably.

In certain embodiments, an antibody or antigen-binding fragment of the present disclosure ineutralizes neutralizing infection by RSV and/or MPV. As used herein, a “neutralizing antibody” is one that “neutralizes,” i.e., prevents, inhibits, reduces, impedes, or interferes with, the ability of a pathogen to initiate and/or perpetuate an infection in a host. The terms “neutralizing antibody” and “an antibody that neutralizes” or “antibodies that neutralize” are used interchangeably herein. In any of the presently disclosed embodiments, a neutralizing antibody or antigen-binding fragment prevents and/or neutralizes a RSV and/or MPV infection in an in vitro model of infection, in an in vivo animal model of infection and/or in a human. A “neutralizing antibody” is also “capable of” or “able to” perform any of the activities ascribed to a neutralizing antibody in this paragraph and an antibody or antigen-binding fragment thereof that “neutralizes” is also “capable of neutralizing” or “able to neutralize.”

In certain embodiments, the antibody or antigen-binding fragment is a RSV-binding and/or neutralizing antibody or antigen-binding fragment as set forth in Table 2, with reference to sequence definitions in Table 1 and the Sequence Listing.

In certain embodiments, the antibody or antigen-binding fragment is a MPV-binding and/or neutralizing antibody or antigen-binding fragment as set forth in Table 2, with reference to sequence definitions in Table 1 and the Sequence Listing.

In certain embodiments, the antibody or antigen binding fragment is a RSV/MPV cross-binding and/or neutralizing antibody or antigen-binding fragment as set forth in Table 2, with reference to sequence definitions in Table 1, Table 20, and the Sequence Listing.

Similar naming conventions are used for the antibodies herein according the the following principles, which apply to antibody and variant designations.

As used herein, “MPH12” without further identification as a variant (e.g. MPH12-v2) refers to an antibody having a MPH12 VH and a MPH-12 VL, as set forth in Tables 1 and 2 and the Sequence Listing, or CRDs associated with MPH12 VH and MPH-12VL as set forth in Tables 1 and 2 and the Sequence Listing and a rIgGI constant region. “MPH-v[#]” refers to a variant of MPH12 as set forth in Tables 2 and 20, with reference to Table 1 and the Sequence Listing, which are collectively called MPH12 “variants.” The first number or only number in a variant refers to a VH variant and a two number “x.y” variant refers to a VH.VL variant.

Typically, unless indicated otherwise by sequences herein, VH.1 refers to the parental VH and VL.1 refers to the parental VL.

MPK[#], as used herein, refers to an antibody whose designation begins with “MPK” in Table 2, with reference to Table 1 and the Sequence Listing. Collectively these antibodies may be referred to as “MPK antibodies.”

Other MP[Letter][#] combinations, as used herein, each refer, in a manner similar to MPK antibodies, to an antibody whose designation begins with “MP[Letter][#]” in Table 2, with reference to Table 1 and the Sequence Listing. Collectively, antibodies with the same MP[Letter][#] combination may be referred to as that specific “MP[Letter][#] antibodies.” “MP[Letter][#]-v[#]. [#]” refers to a variant of an MP[Letter][#] as set forth in Tables 2 and 20, with reference to Table 1 and the Sequence Listing, which are collectively called MP[Letter][#]“variants.” The first number in a variant refers to a VH variant and the second number refers to a VL variant. For example, MPK190-v1.3 refers to a MPK190 antibody having VH.1 and VL.3 variants of parental MPK190. MPK176-v1.3 refers to an antibody having the VH.1 and VL.3 variants of parental MPK176. MPK 176-v4.3 refer to an antibody having the VH.4 and VL.3 variants of parental MPK176. MPK201-v1.2 refers to an antibody having the VH.1 and VL.2 variants of parental MPK201. MPK201-v4.1 refers to an antibody having the VH.4 and VL.2 variants of parental MPK201.

Certain antibodies are named as a variant of the parental antibody without reference to specific VH and VL variants. These antibodies are designated MP[Letter][#]-v[#], where the v[#] is a single number, and may be considered a parental antibody and may be referred to without the “v[#] designation. MPK65-v2 is an example of such an antibody. VH and VL variants of such antibodies are designated the same as in other MP[Letter][#] antibodies. For example, MPK65-v2-v1.2 refers to an antibody having VH.1 and VL.2 variants of parental MPK65-v2. MPK65-v2-v3.1 refers to an antibody having VH.3 and VL.1 variants of parental MPK65-v2. MPK65-v2 is sometimes referred to simply as “MPK65.”

Certain comparator antibodies or antibodies that may be used in various compbinations with MPH, MPK, MPM, MPO, MPP, MPR, or other MP[Letter][#] antibodies, including HMB[#], and RSD5, are described in Tables 3 and 4, with reference to Table 1 and the Sequence Listing. MPE33 and MPE8 are described in Corti et al. Nature. 2013 Sep. 19; 501(7467):439-43. doi: 10.1038/nature12442. Epub 2013 Aug. 18. MPF5, and RSD5 are descrbed in Jones et al. PLoS Patho. 15(7):e1007944 (2019); doi:10.1371/journal.ppat.1007944.

Antibodies havein the same VH and VL as these reference antibodies, but variations in the Fc regions, may also be used in their place, as similar binding properties would be expected.

In certain embodiments, the antibody or antigen-binding fragment is human, humanized, or chimeric.

In certain embodiments, the antibody or antigen-binding fragment comprises one or more of a VH and/or VL, and/or two or more of a CDRH1, CDRH2, CDRH3, CDRL1, CRL2, and/or CDRL3 as described in SEQ ID NOs.:1-530, 811-862, and 891-903 and binds to RSV-F in a pre-fusion conformation with a KD (in M) of 1.0E−12 or less, 1.0E−11 or less, 4.5E−11 or less, 1.0E−10 or less, 1.0E−9 or less, 1.0E−8 or less, or 1.0E−7 or less, or in a range of 1.0E−12 to 1.0E−7, 1.0E−12 to 1.0E−8, 1.0E−12 to 1.0E−9, 1.0E−12 to 1.0E−10, or 1.0E−12 to 1.0E−11 wherein, optionally the binding is assessed by surface plasmon resonance (SPR). In some embodiments, binding is any of the preceding values or ranges that are 1.0E−9 or less, which is high affinity binding in accordance with the present disclosure.

In certain embodiments, the antibody or antigen-binding fragment comprises one or more of a VH and/or VL, and/or two or more of a CDRH1, CDRH2, CDRH3, CDRL1, CRL2, and/or CDRL3 as described in SEQ ID NOs.:531-556 and binds to RSV-F in a pre-fusion conformation with a KD (in M) of 1.0E−10 or less, 7.5E−10 or less, 1.0E−9 or less, or 1.5E−9 or less, or in a range of 1.0E−10 to 1.5E−9, 1.0E−10 to 1.0E−9, 1.0E−10 to 7.5E−10, or 7.5E−10 to 1.5E−9, wherein, optionally the binding is assessed by surface plasmon resonance (SPR). In some embodiments, binding is any of the preceding values or ranges that are 1.0E−9 or less, which is high affinity binding in accordance with the present disclosure.

In certain embodiments, the antibody or antigen-binding fragment comprises one or more of a VH and/or VL, and/or two or more of a CDRH1, CDRH2, CDRH3, CDRL1, CRL2, and/or CDRL3 as described in SEQ ID NOs.:1-530, 811-862, and 891-903 and binds to MPV-F D280 with a KD (in M) of 1.0E−12 M or less, 1.0E−11 or less, 4.5E−11 or less, 1.0E−10 or less, 1.0E−9 or less, 8.0E−9 or less, 1.0E−8 or less, or 1.0E−7 or less, or in a range of 1.0E−12 to 1.0E−7, 1.0E−12 to 8.0E−9, 1.0E−12 to 1.0E−10, 1.0E−12 to 4.5E−11, 1.0E−12 to 1.0E−11, or 4.5E−11 to 8.0E−9, wherein, optionally the binding is assessed by surface plasmon resonance (SPR). In some embodiments, binding is any of the preceding values or ranges that are 1.0E−9 or less, which is high affinity binding in accordance with the present disclosure.

In certain embodiments, the antibody or antigen-binding fragment comprises one or more of a VH and/or VL, and/or two or more of a CDRH1, CDRH2, CDRH3, CDRL1, CRL2, and/or CDRL3 as described in SEQ ID NOs.:531-556 and binds to MPV-F D280 with a KD (in M) of 1.0E−12 or less, 1.0E−11 or less, 4.5E−11 or less, or 1.0E−10 or less, or in a range of 1.0E−12 tol.0E−11, 1.0E−12 to 4.5E−11, or 1.0E−12 to 1.0E−10, wherein, optionally the binding is assessed by surface plasmon resonance (SPR).

In certain embodiments, the antibody or antigen-binding fragment comprises one or more of a VH and/or VL, and/or two or more of a CDRH1, CDRH2, CDRH3, CDRL1, CRL2, and/or CDRL3 as described in SEQ ID NOs.:1-530, 811-862, and 891-903 and binds to MPV-F N280 with a KD (in M) of 1.0E−12 or less, 1.0E−11 or less, 5.8E−11 or less, 1.0E−10 or less, 1.0E−9 or less, 8.0E−9 or less, 1.0E−8 or less, or 1.0E−7 or less, or in a range of 1.0E−12 to 1.0E−7, 1.0E−12 to 1.0E−11, 1.0E−12 to 1.0E−10, 1.0E−12 to 1.0E−9, 1.0E−12 to 1.0E−8, 1.0E−12 to 1.0E−7, or 1.0E−12 to 5.8E−11, wherein, optionally the binding is assessed by surface plasmon resonance (SPR). In some embodiments, binding is any of the preceding values or ranges that are 1.0E−9 or less, which is designated high affinity binding in accordance with the present disclosure.

In certain embodiments, the antibody or antigen-binding fragment comprises one or more of a VH and/or VL, and/or two or more of a CDRH1, CDRH2, CDRH3, CDRL1, CRL2, and/or CDRL3 as described in SEQ ID NOs.:531-556 and binds to MPV-F N280 with a KD (in M) of 1.0E−10 or less, 1.0E−9 or less, 2.5 E−9 or less, or 1.0E−8 or less, or in a range of 1.0E−10 to 1.0E−8, 1.0E−10 to 1.0E−9, or 1.0E−10 to 2.5E−9, wherein, optionally the binding is assessed by surface plasmon resonance (SPR). In some embodiments, binding is any of the preceding values or ranges that are 1.0E−9 or less, which is high affinity binding in accordance with the present disclosure.

In certain embodiments, the antibody or antigen-binding fragment comprises one or more of a VH and/or VL, and/or two or more of a CDRH1, CDRH2, CDRH3, CDRL1, CRL2, and/or CDRL3 as described in SEQ ID NOs.:1-530, 811-862, and 891-903 and neutralizes RSV as measured in vitro with an IC50 of 30 ng/ml or less, 15 ng/ml or less, 10 ng/ml or less, 2.0 ng/ml or less, 1.0 ng/ml or less, or 0.2 ng/ml or less, or in a range of 0.2 ng/ml to 30 ng/ml, 0.2 ng/ml to 15 ng/ml, 0.2 ng/ml to 2.0 ng/ml, 1.0 ng/ml to 30 ng/ml, or 2.0 ng/ml to 15 ng/ml.

In certain embodiments, the antibody or antigen-binding fragment comprises one or more of a VH and/or VL, and/or two or more of a CDRH1, CDRH2, CDRH3, CDRL1, CRL2, and/or CDRL3 as described in SEQ ID NOs.:1-530, 811-862, and 891-903 and neutralizes MPV as measured in vitro with an IC50 of 30 ng/ml or less, 15 ng/ml or less, 10 ng/ml or less, 7.0 ng/ml or less, 6.0 ng/ml or less, 5 ng/ml or less, or 1.5 ng/ml or less, or in a range of 1.0 ng/ml to 30 ng/ml, 1.0 ng/ml to 15 ng/ml, 1.0 ng/ml to 10 ng/ml, 1.0 ng/ml to 7.0 ng/ml, 1.0 ng/ml to 5.0 ng/ml, 5.0 ng/ml to 30 ng/ml, 5.0 ng/ml to 15 ng/ml, 7.0 ng/ml to 30 ng/ml, or 7.0 ng/ml to 15 ng/ml.

In certain embodiments, the antibody or antigen-binding fragment comprises one or more of a VH and/or VL, and/or two or more of a CDRH1, CDRH2, CDRH3, CDRL1, CRL2, and/or CDRL3 as described in SEQ ID NOs.:531-556 and neutralizes RSV as measured in vitro with an IC50 of 30 ng/ml or less, 15 ng/ml or less, 10 ng/ml or less, 8.0 ng/ml or less, 5.0 ng/ml or less, or 3.5 ng/ml or less, or in a range of 3.0 ng/ml to 30 ng/ml, 3.0 ng/ml to 15 ng/ml, 3.0 ng/ml to 10 ng/ml, 3.0 ng/ml to 8 ng/ml, 3.0 ng/ml to 5.0 ng/ml, 5.0 ng/ml to 30 ng/ml, or 5.0 ng/ml to 15 ng/ml.

In certain embodiments, the antibody or antigen-binding fragment comprises one or more of a VH and/or VL, and/or two or more of a CDRH1, CDRH2, CDRH3, CDRL1, CRL2, and/or CDRL3 as described in SEQ ID NOs.:531-556 and neutralizes MPV as measured in vitro with an IC50 of 30 ng/ml or less, 15 ng/ml or less, 10 ng/ml or less, 5.0 ng/ml or less, or 3.8 ng/ml or less, or in a range of 3.5 ng/ml to 30 ng/ml, 3.5 ng/ml to 15 ng/ml, 3.5 ng/ml to 10 ng/ml, 3.5 ng/ml to 5.0 ng/ml, 5.0 ng/ml to 30 ng/ml, or 5.0 ng/ml to 15 ng/ml.

In certain embodiments, the in vitro measurement includes an ELISA.

In certain embodiments, the antibody or antigen-binding fragment binds to or neutralizes two or more of RSV-F, MPV-F D280, and MPV-F N280 with a KD or IC50 for each protein as indicated above.

In certain embodiments, the antibody or antigen-binding fragment binds to or neutralizes DS-Cav1, or two or more of DS-Cav1, RSV-F, MPV-F D280, and MPV-F N280 with a KD or IC50 for each protein as indicated above.

In certain embodiments, the RSV-F comprises DS-Cav1, a stabilized trimer of the pre-fusion conformation of the RSV-F protein that comprises the amino acid mutation(s): S155C, S190F, V207L, and S290C wherein, optionally, the RSV comprises strain B18537 (NCBI:txid11251).

In certain embodiments, the antibody or antigen-binding fragment activates a human FcγRIIIa (or is “capable of” or “able to” activate a human FcγRIIIa). In further embodiments, activation is as determined using a host cell (optionally, a Jurkat cell) comprising: (i) the human FcγRIIIa (optionally, a F158 allele); and (ii) a NFAT expression control sequence operably linked to a sequence encoding a reporter, such as a luciferase reporter, following incubation (e.g., of 23 hours) of the antibody or antigen-binding fragment with atarget cell (e.g., a Expi293 cell) transiently transfected with RSV-F and/or MPV-F. In still further embodiments, activation is as determined following an incubation (optionally, for about 23 hours) of the antibody or antigen-binding fragment with the target cell transiently transfected with RSV-F and/or MPV-F.

In certain embodiments, the antibody or antigen-binding fragment neutralizes infection by RSV and/or MPV. In certain embodiments, the RSV and/or the MPV is antiviral-resistant (e.g. Ribavirin-resistant). In certain embodiments, the MPV does not contain the D280N mutation in its fusion glycoprotein. In certain embodiments, the MPV does contain the D280N mutation in its fusion glycoprotein.

In certain embodiments, the antibody or antigen-binding fragment treats and/or prevents (or is “capable of treating and/ore preventing” or “able to treat and/or prevent”) (i) a RSV infection and/or (ii) a MPV infection in a subject.

In certain embodiments, the antibody or antigen-binding fragment extends survival of (or is “capable of extending survival of” or “able to extent survival or”) a subject having a RSV infection and/or a MPV infection.

In certain embodiments, the antibody or antigen-binding fragment reduces (or is “capable of reducing” or “able to reduce”) viral loads in the nasal tissue, nasal homogenates, bronchoalveolar fluid (BALF), and/or lung homogenates of a subject having a RSV infection and/or a MPV infection.

In certain embodiments, the antibody or antigen-binding fragment reduces (or is “capable of reducing” or “able to reduce”) infection-associated pulmonary pathology of a subject having a RSV infection and/or a MPV infection.

In any of the above embodiments, the antibody or antigen-binding fragment may treat and/or attenuate (or be “capable of treating and/or attenuating” or “able to treat and/or attenuate”) an infection by a MPV virus expressing MPV-F D280, such as MPV-F wt, and/or by a MPV virus expressing MPV-F N280. Such an antibody may be therapeutically administered to a human subject without the need to ascertain whether a MPV virus infecting the subject contains the MPV-F N280 mutation.

Similarly, if the antibody or antigen-binding fragment may treat and/or attenuate (or be “capable of treating and/or attenuating” or “able to treat and/or attenuate”) an infection by both RSV and MPV, optionally MPV both with or without the D280N mutation, the antibody or antigen-binding fragment may be therapeutically administered to a human subject with to the need to ascertain whether the virus infecting the subject has a RSV infection or MPV infection, or, optionally, whether the virus contains the MPV-F N280 mutation.

In some embodiments, antibodies or antigen-binding fragments thereof of the present disclosure may bind to, neutralize, neutralilze infection by, prevent infection by, treat infection by, reduce viral load of, reduce infection-associated pulmonary pathology or, or any combinations thereof, of multiple RSV and/or MPV strains (also sometimes referred to as RSV and/or MPV types or subtypes).

For example, a RSV-binding antibody or antigen-binding fragment thereof may bind to, neutralize, neutralilze infection by, prevent infection by, treat infection by, reduce viral load of, reduce infection-associated pulmonary pathology or, or any combinations thereof, of both RSV A and RSV B strains. A RSV-binding antibody may bind to, neutralize, neutralilze infection by, prevent infection by, treat infection by, reduce viral load of, reduce infection-associated pulmonary pathology or, or any combinations thereof, of multiple subtypes of RSV A strains. A RSV-binding antibody or antigen-binding frantment thereof may bind to, neutralize, neutralilze infection by, prevent infection by, treat infection by, reduce viral load of, reduce infection-associated pulmonary pathology or, or any combinations thereof, of multiple subtypes of RSV B strains.

Also for example, a MPV-binding antibody or antigen-binding fragment thereof may bind to, neutralize, neutralilze infection by, prevent infection by, treat infection by, reduce viral load of, reduce infection-associated pulmonary pathology or, or any combinations thereof, of both MPV A and MPV B strains. A MPV-binding antibody may bind to, neutralize, neutralilze infection by, prevent infection by, treat infection by, reduce viral load of, reduce infection-associated pulmonary pathology or, or any combinations thereof, of multiple subtypes of MPV A strains, such as MPV A1 strains, MPV A2 strains (including A2a, A2b, or both subtypes), or combinations thereof Δ MPV-binding antibody or antigen-binding frantment thereof may bind to, neutralize, neutralilze infection by, prevent infection by, treat infection by, reduce viral load of, reduce infection-associated pulmonary pathology or, or any combinations thereof, of multiple subtypes of MPV B strains, such as MPV B1 strains, MPV B2 strains, or combinations thereof.

In one embodiment, an antibody or antigen-binding fragment thereof of the present dislclosure may bind to, neutralize, neutralilze infection by, prevent infection by, treat infection by, reduce viral load of, reduce infection-associated pulmonary pathology or, or any combinations thereof, of a RSV A strain, a RSV B strain, and a MPV A strain.

In another embodiment, an antibody or antigen-binding fragment thereof of the present disclosure may bind to, neutralize, neutralilze infection by, prevent infection by, treat infection by, reduce viral load of, reduce infection-associated pulmonary pathology or, or any combinations thereof, of a RSV A strain, a RSV B strain, and a MPV B strain.

In another embodiment, an antibody or antigen-binding fragment thereof of the present disclosure may bind to, neutralize, neutralilze infection by, prevent infection by, treat infection by, reduce viral load of, reduce infection-associated pulmonary pathology or, or any combinations thereof, of a RSV A strain, a MPV A strain, and a MPV B strain.

In another embodiment, an antibody or antigen-binding fragment thereof of the present disclosure may bind to, neutralize, neutralilze infection by, prevent infection by, treat infection by, reduce viral load of, reduce infection-associated pulmonary pathology or, or any combinations thereof, of a RSV A strain, a MPV A strain, and a MPV B strain.

In another embodiment, an antibody or antigen-binding fragment thereof may bind to, neutralize, neutralilze infection by, prevent infection by, treat infection by, reduce viral load of, reduce infection-associated pulmonary pathology or, or any combinations thereof, of a RSV B strain, a MPV A strain, and a MPV B strain.

In one embodiment, an antibody or antigen-binding fragment thereof of the present disclosure may bind to, neutralize, neutralilze infection by, prevent infection by, treat infection by, reduce viral load of, reduce infection-associated pulmonary pathology or, or any combinations thereof, of any combination of subcombination of the following viral strains: RSV A, RSV B, MPV A1, MPV A2 (MPV A2a, MPV A2b, or both), MPV B1, and MPV B2. In a specific embodiment an antigen or antigen-binding fragment thereof of the present disclosure may bind to, neutralize, neutralilze infection by, prevent infection by, treat infection by, reduce viral load of, reduce infection-associated pulmonary pathology or, or any combinations thereof, of RSV A, RSV B, MPV A1, MPV A2, MPV B1, and MPV B2.

In any of the above embodiments, an antibody or antigen-binding fragment that may may treat and/or attenuate (or be “capable of treating and/or attenuating” or “able to treat and/or attenuate”) an infection by both RSV A and RSV B strains may be therapeutically administered to a human subject without the need to ascertain the viral subtype of a RSV virus infecting the subject.

Similarly, an antibody or antigen-binding fragment that may may treat and/or attenuate (or be “capable of treating and/or attenuating” or “able to treat and/or attenuate”) an infection by both MPV A and MPV B strains may be therapeutically administered to a human subject without the need to ascertain the viral subtype of a MPV virus infecting the subject.

Additionally, an antibody or antigen-binding fragment that may may treat and/or attenuate (or be “capable of treating and/or attenuating” or “able to treat and/or attenuate”) an infection by both RSV A and RSV B strains and both MPV A and MPV B strains may be therapeutically administered to a human subject without the need to ascertain whether RSV or MPV is infecting the subject.

In one embodiment, the antibody or antigen-binding fragment thereof of the present disclosure binds to Site III of the RSV F protein. In a more specific embodiment, such an antibody or antigen-binding fragment thereof may bind to and treat infection by both RSV and MPV.

In one embodiment, the antibody or antigen-binding fragment thereof of the present disclosure binds to Site 0 of RSV F protein. In a more specific embodiment, such an antibody or antigen-binding fratment thereof may bind to and treat infection by RSV.

In one embodiment, the antibody or antigen-binding fragment thereof of the present disclure binds to Site IV of the RSV F protein. In a more specific embodiment, such an antibody or antigen-binding fragment thereof may bind to and treat infection by RSV.

For each embodiment of the antibodies, antigen-binding fragment, and compositons above, in parallel embodiments, a combination of two or more antibodies or antigen-binding fragments thereof of the present disclosure, either as separate antibodies in a single composition, or in a bispecific antibody, may have the same RSV and MPV binding, neutralizing, infection preventing and/or treating, and other recited properties.

In certain embodiments, the antibody or antigen-binding fragment fragment (e.g., comprising an IgGI isotype) has an in vivo half-life in a mouse (e.g., a tg32 mouse) in a range from about 10 days to about 17 days, about 10 days to about 16 days, about 10 days to about 15 days, about 10 days to about 14 days, about 10 days to about 13 days, about 10 days to about 12 days, about 11 days to about 17 days, about 11 days to about 16 days, about 11 days to about 15 days, about 11 days to about 14 days, about 11 days to about 13 days, about 11 days to about 12 days, about 12 days to about 17 days, about 12 days to about 16 days, about 12 days to about 15 days, about 12 days to about 14 days, about 12 days to about 13 days, about 12.5 days to about 16 days, about 12.5 days to about 15.5 days, about 12.5 days to about 15 days, about 12.5 days to about 14.5 days, about 12.5 days to about 14 days, about 12.5 days to about 13.5 days, about 12.5 days to about 13 days, about 13 days to about 16 days, about 13 days to about 15.5 days, about 13 days to about 15 days, about 13 days to about 14.5 days about 13 days to about 14 days, about 13 days to about 13.5 days, about 13.5 days to about 16 days, about 13.5 days to about 15.5 days, about 13.5 days to about 15 days, about 13.5 days to about 14.5 days, about 13.5 days to about 14 days, about 14 days to about 16 days, about 14 days to about 15.5 days, about 14 days to about 15 days, about 14 days to about 14.5 days, about 14.5 days to about 16 days, about 14.5 days to about 15.5 days, about 14.5 days to about 15 days, about 15 days to about 16 days, about 15 days to about 15.5 days, about 15.5 days to about 16 days, or of about 10, 11, 12, 12.5, 13, 13.5, 14, 14.5, 15, 15.5, 16, or 17 days.

In some embodiments, an antibody or antigen-binding fragment of the present disclosure may reduce weight loss in RSV-infected mice at least as well as nirsevimab when administered in a similar manner to similar mice in a similar stage of RSV infection.

In some embodiments, an antibody or antigen-bindiding fratment of the present disclosure may increase surfival of RSV-infected mice at least as well as nirsevimab, or an antibody having the same VH and VL as Niservimab, when administered in a similar manner to similar mice in a similar stage of RSV infection.

Terms understood by those in the art of antibody technology are each given the meaning acquired in the art, unless expressly defined differently herein. For example, the term “antibody” refers to an intact antibody comprising two or more heavy (H) chains and two light (L) chains inter-connected by disulfide bonds, as well as any antigen-binding portion or fragment of an intact antibody that has or retains the ability to bind to the antigen target molecule recognized by the intact antibody, such as an scFv, Fab, or Fab′2 fragment. Thus, the term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments. The term also encompasses intact or full-length antibodies, including antibodies of any class or sub-class, including IgG and sub-classes thereof (IgG1, IgG2, IgG3, IgG4), IgM, IgE, IgA, and IgD.

An antibody or antigen-binding fragment, may be of any allotype or combination of allotypes. “Allotype” refers to the allelic variation found among the IgG subclasses. For example, an allotype may comprise G1m1 (or G1m(a)), G1m2 (or G1m(x)), G1m3 (or G1m(f)), G1m17 (or Gm(z))m), G1m27, and/or G1m28 (G1m27 and G1m28 have been described as “alloallotypes”).

The G1m3 and G1m17 allotypes are located at the same position in the CHi domain (position 214 according to EU numbering). G1m3 comprises R214 (EU), while G1m17 comprises K214 (EU). The G1m1 allotype is located in the CH3 domain (at positions 356 and 358 (EU)) and refers to the replacements E356D and M358L. The G1m2 allotype refers to a replacement of the alanine in position 431 (EU) by a glycine. G1m allotypes, alloallotypes, and features thereof are known in the art and described at, for example, <www.imgt.org/IMGTrepertoire/Proteins/allotypes/human/IGH/IGHC/G1m_allotypes.html>> and Lefranc, M.-P. and Lefranc, G. Human Gm, Km and Am allotypes and their molecular characterization: a remarkable demonstration of polymorphism In: B. Tait, F. Christiansen (Eds.), Immunogenetics, chap. 34, Humana Press, Springer, New York, USA. Methods Mol.

Biol. 2012; 882, 635-680. PMID: 22665258, LIGM: 406, the contents and allotypes and allotype information of which are incorporated herein by reference.

The G1m1 allotype may be combined, for example, with the G1m3, G1m17, G1m27, G1m2, and/or G1m28 allotype. In some embodiments, an allotype is G1m3 with no G1m1 (G1m3,−1). In some embodiments, an allotype is G1m17,1 allotype. In some embodiments, an allotype is G1m3,1. In some embodiments, an allotype is G1m17 with no G1m1 (G1m17,−1). Optionally, these allotypes may be combined (or not combined) with the G1m2, G1m27 or G1m28 allotype. For example, an allotype may be G1m17,1,2.

In some embodiments, an antibody or antigen-binding fragment of the present disclosure comprises a G1m3 allotype or a G1m3,1 allotype. In some embodiments, an antibody or antigen-binding fragment of the present disclosure comprises a G1m3 allotype and comprises M428L and N434S or M428L and N434A mutations or any other mutation(s) that enhance binding to a human FcRn, such as those described herein. In some embodiments, an antibody or antigen-binding fragment of the present disclosure comprises a G1m3,1 allotype and comprises M428L and N434S or M428L and N434A mutations or any other mutation(s) that enhance binding to a human FcRn, such as those described herein. In some embodiments, an antibody or antigen-binding fragment of the present disclosure comprises a G1m17, 1 allotype. In some embodiments, an antibody or antigen-binding fragment of the present disclosure comprises a G1m17, 1 allotype and comprises M428L and N434S or M428L and N434A mutations or any other mutation(s) that enhance binding to a human FcRn, as described further herein.

The terms “VL” or “VL” and “VH” or “VH” refer to the variable binding region from an antibody light chain and an antibody heavy chain, respectively. In certain embodiments, a VL is a kappa (x) class (also “VK” herein). In certain embodiments, a VL is a lambda (Q) class.

The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary structure by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FR1-HCDR1-FR2-HCDR2-FR3-HCDR3-FR4; and an antibody VL comprises four FRs and three CDRs as follows: FR1-LCDR1-FR2-LCDR2-FR3-LCDR3-FR4.

In general, the VH and the VL together form the antigen-binding site through their respective CDRs. In certain embodiments, one or more CDRs do not contact antigen and/or do not contribute energetically to antigen binding.

As used herein, a “variant” of a CDR refers to a functional variant of a CDR sequence having up to 1-3 amino acid substitutions (e.g., conservative or non-conservative substitutions), deletions, or combinations thereof.

Numbering of CDR and framework regions may be according to any known method or scheme, such as the Kabat, Chothia, EU, Martin (Enhanced Chothia), Contact, AbM, CCG, EU, or AHo, numbering method or a combination of two or more of theseIMGT, North, and AHo numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J Mol. Biol. 196:901-917 (1987)); Lefranc et al., Dev. Comp. Immunol. 27:55, 2003; Honegger and Plückthun, J. Mol. Bio. 309:657-670 (2001)). Equivalent residue positions can be annotated and for different molecules to be compared using Antigen receptor Numbering And Receptor Classification (ANARCI) software tool (2016, Bioinformatics 15:298-300). Accordingly, identification of CDRs of a variable domain (VH or VL) sequence as provided herein according to one numbering scheme is not exclusive of an antibody comprising CDRs ofthe same variable domain as determined using a different numbering scheme.

In certain embodiments, an antibody or antigen-binding fragment that binds RSV-F is provided that comprises the CDRs of a VH sequence according to any one of SEQ ID NOs.: 2, 136, 146, 159, 169, 175, 181, 189, 196, 202, 210, 215, 225, 233, 243, 250, 254, 261, 271, 277, 284, 293, 300, 309, 316, 321, 327, 332, 342, 347, 352, 357, 362, 369, 727, 737, 746, 755, 765, 775, 784, 794, 804, 813, 817, 820, 823, 826, 828, 831, 834, 837, 840, 843, 883, 886, 889, 893, 896, 899, 901, 903, 129, 12, 38, 46, 53, 59, 69, 120, 73, 79, 86, 95, 22, 111,30,378,386,395, 404, 412, 416, 422, 426, 431, 440, 451, 458, 463, 470, 475, 480, 485, 491, 497, 532, 537, 539, 542, 545, 547, 550, 504, 513, 524, 527, 702, 707, 712, and 716, and/or of a VL sequence according to any one of SEQ ID NOs.: 77, 141, 150, 155, 164, 172, 178, 185, 192, 199, 205, 212, 220, 229, 238, 247, 252, 257, 266, 274, 282, 288, 296, 305, 312, 319, 324, 330, 337, 345, 349, 355, 360, 367, 374, 732, 742, 751, 760, 770, 780, 789, 799, 808, 847, 851, 855, 858, 860, 862, 865, 868, 870, 873, 875, 877, 879, 881, 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 553, 558, 560, 563, 567, 570, 572, 574, 704, and 710, as determined using any known CDR numbering method, including the Kabat, Chothia, EU, Martin (Enhanced Chothia), Contact, AbM, CCG, EU, or AHo, numbering method or a combination of two or more of theseIMGT, Martin (Enhanced Chothia), Contact, North, and AHo numbering methods methods, including as determined by a combination of any two or more of these numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software.

In certain embodiments, an antibody or antigen-binding fragment that binds RSV-F is provided that comprises the CDRs of a VH sequence according to any one of SEQ ID NOs.: 2, 136, 146, 159, 169, 175, 181, 189, 196, 202, 210, 215, 225, 233, 243, 250, 254, 261, 271, 277, 284, 293, 300, 309, 316, 321, 327, 332, 342, 347, 352, 357, 362, 369, 727, 737, 746, 755, 765, 775, 784, 794, 804, 813, 817, 820, 823, 826, 828, 831, 834, 837, 840, 843, 883, 886, 889, 893, 896, 899, 901, and 903 and/or of a VL sequence according to any one of SEQ ID NOs.: 7, 141, 150, 155, 164, 172, 178, 185, 192, 199, 205, 212, 220, 229, 238, 247, 252, 257, 266, 274, 282, 288, 296, 305, 312, 319, 324, 330, 337, 345, 349, 355, 360, 367, 374, 732, 742, 751, 760, 770, 780, 789, 799, 808, 847, 851, 855, 858, 860, 862, 865, 868, 870, 873, 875, 877, 879, and 881 as determined using any known CDR numbering method, including the Kabat, Chothia, EU, Martin (Enhanced Chothia), Contact, AbM, CCG, EU, or AHo, numbering method or a combination of two or more of theseIMGT, Martin (Enhanced Chothia), Contact, North, and AHo numbering methods, including as determined by a combination of any two or more of these numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software).

In some embodiments that bind MPK only, the CDRs comprise or consist of the CDRs of MPK15.

More specifically, the VH and VL for an antibody or antigen-binding fragment that binds RSV-F can comprise or consist of the VH and VL amino acid sequences, respectively, of anti-RSV MPK antibodies or MPH antibodies in Table 2, and have corresponding amino acid sequences set forth in Table 1 and the Sequence Listing. Specifically, the CDRs comprise or consist of the CDRs of MPK44, MPK65-v2, MPK161-v2, MPK163, MPK165, MPK167, MPK168, MPK169-v2, MPK170, MPK171-v1, MPK171-v2, MPK173, MPK175, MPK176, MPK177, MPK178, MPK179-v4, MPK180, MPK181, MPK182, MPK185, MPK186, MPK187, MPK188, MPK189, MPK191, MPK193, MPK194-v2, MPK195, MPK197, MPK198, MPK201, MPK202, MPK203, MPM10, MPM2, MPM8, MPO1, MPO7, MPP1, MPP2, MPR16, MPR19-v2, MPM10, MPM2, MPM8, MPO1, MPO7, MPP1, MPP2, MPR16, MPR19-v2, MPK65-v (any one of 1-7).(any one 1-2), MPK201-v (any one of 1-6).(any one of 1-2), MPK176-v (any one of 1-6).(any one of 5), or MPM2-v (any one of 1, 2, 4, 5).(any one of 1-9), e.g., MPK176-v1.3, MPK176-v4.3, MPK201-v1.2, MPK201-v4.1, MPK65v2-v1.2, or MPK65v2-v3.1. It will be understood that, for example, MPK201-v1.2 comprises the VH of MPK201 VH.1 (SEQ ID NO.:357) and the VL of MPK201 VL.2 (SEQ ID NO.:847).

In some embodiments, the antibody or antigen-binding fragment comprises the six CDRs of MPK176-v1.3, MPK176-v4.3, MPK201-v1.2, MPK201-v4.1, MPK65v2-v1.2, or MPK65v2-v3.1.

More specifically, the VH and VL for an antibody or antigen-binding fragment that binds RSV-F comprise or consist of a VH and VL having the following sequences: 1) SEQ ID NOs.: 2 and 6; 2) SEQ ID NOs.: 136 and 141; 3) SEQ ID NOs.: 146 and 150; 4) SEQ ID NOs.: 146 and 155; 5) SEQ ID NOs.: 159 and 164; 6) SEQ ID NOs.: 169 and 172; 7) SEQ ID NOs.: 175 and 178; 8) SEQ ID NOs.: 181 and 185; 9) SEQ ID NOs.: 189 and 192; 10) SEQ ID NOs.: 196 and 199; 11) SEQ ID NOs.: 202 and 205; 12) SEQ ID NOs.: 210 and 212; 13) SEQ ID NOs.: 215 and 220; 14) SEQ ID NOs.: 225 and 229; 15) SEQ ID NOs.: 233 and 238; 16) SEQ ID NOs.: 243 and 247; 17) SEQ ID NOs.: 250 and 252; 18) SEQ ID NOs.: 254 and 257; 19) SEQ ID NOs.: 261 and 266; 20) SEQ ID NOs.: 271 and 274; 21) SEQ ID NOs.: 277 and 282; 22) SEQ ID NOs.: 284 and 288; 23) SEQ ID NOs.: 293 and 296; 24) SEQ ID NOs.: 300 and 305; 25) SEQ ID NOs.: 309 and 312; 26) SEQ ID NOs.: 316 and 319; 27) SEQ ID NOs.: 321 and 324; 28) SEQ ID NOs.: 327 and 330; 29) SEQ ID NOs.: 332 and 337; 30) SEQ ID NOs.: 342 and 345; 31) SEQ ID NOs.: 347 and 349; 32) SEQ ID NOs.: 352 and 355; 33) SEQ ID NOs.: 357 and 360; 34) SEQ ID NOs.: 362 and 367; 35) SEQ ID NOs.: 369 and 374, 36) SEQ ID NOs.: 883 and 742; 37) SEQ ID NOs.: 737 and 742; 38) SEQ ID NOs.: 727 and 732; 39) SEQ ID NOs.: 746 and 751; 40) SEQ ID NOs.: 755 and 760; 41) SEQ ID NOs.: 765 and 770; 42) SEQ ID NOs.: 775 and 770; 43) SEQ ID NOs.: 784 and 789; 44) SEQ ID NOs.: 794 and 799; 45) SEQ ID NOs.: 804 and 808; 46) SEQ ID NOs.: 233 and 858; 47) SEQ ID NOs.: 837 and 858; 48) SEQ ID NOs.: 357 and 847; 49) SEQ ID NOs.: 899 and 360; 50) SEQ ID NOs.: 136 and 851; 51) SEQ ID NOs.: 817 and 141; 52) SEQ ID NOs.: 883 and 742; 53) SEQ ID NOs.: 886 and 742; or 54) SEQ ID NOs.: 883 and 875, respectively.

More specifically, the VH and VL for an antibody or antigen-binding fragment that binds MPV-F can comprise or consist of the VH and VL identified for anti-MPV MPK antibodies or MPH antibodies in Table 2, and have corresponding amino acid sequences set forth in Table 1 and the Sequence Listing.

In some embodiments, an antibody or antigen-binding fragment that binds MPV-F is provided that can comprise the CDRs of a VH sequence according to any one of SEQ ID NOs.:101, 129, 12, 38, 46, 53, 59, 69, 120, 73, 79, 86, 95, 22, 111, 30, 378, 386, 395, 404, 412, 416, 422, 426, 431, 440, 451, 458, 463, 470, 475, 480, 485, 491, 497, 532, 537, 539, 542, 545, 547, 550, 504, 513, 524, 527, 702, 707, 712, and 716, and/or a VL sequence according to any one of SEQ ID NOs.: 106, 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 553, 558, 560, 563, 567, 570, 572, 574, 704, and 710, respectively as determined using any known CDR numbering method, including the Kabat, Chothia, EU, Martin (Enhanced Chothia), Contact, AbM, CCG, EU, or AHo, numbering method or a combination of two or more of theselMGT, Martin (Enhanced Chothia), Contact, North, and AHo numbering methods, including as determined by a combination of any two or more of these numbering methods. In certain embodiments, CDRs are according to the IMGT numbering method including as determined by a combination of any two or more of these numbering methods. In certain embodiments, CDRs are according to the antibody numbering method developed by the Chemical Computing Group (CCG); e.g., using Molecular Operating Environment (MOE) software.

More specifically, the VH and VL for an antibody or antigen-binding fragment that binds MPV-F can comprise or consist of a VH and VL having the sequences of SEQ ID NOs.: 101 and 106, respectively.

In certain embodiments, an antibody or antigen-binding fragment that binds RSV-F and/or MPV-F is provided that can comprise the CDRs of a VH sequence according to any one of SEQ ID NOs.: 129, 12, 38, 46, 53, 59, 69, 120, 73, 79, 86, 95, 22, 111, 30, 378, 386, 395, 404, 412, 416, 422, 426, 431, 440, 451, 458, 463, 470, 475, 480, 485, 491, 497, 532, 537, 539, 542, 545, 547, 550, 504, 513, 524, 527, and 702, 707, 712, and 716, and/or of a VL sequence according to any one of SEQ ID NOs.: 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 553, 558, 560, 563, 567, 570, 572, 574, 704, and 710, as determined using any known CDR numbering method, including the Kabat, Chothia, EU, Martin (Enhanced Chothia), Contact, AbM, CCG, EU, or AHo, numbering method or a combination of two or more of theseIMGT, Martin (Enhanced Chothia), Contact, North, and AHo numbering methods, including as determined by a combination of any two or more of these numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software.

In certain embodiments, an antibody or antigen-binding fragment that binds RSV-F and/or MPV-F is provided that can comprise CDRs of a VH sequence according to any one of SEQ ID NOs.: 532, 537, 539, 542, 545, 547, and 550 and/or of a VL sequence according to any one of SEQ ID NOs.: 553, 558, 560, 563, 567, 570, 572, and 574 as determined using any known CDR numbering method, including the Kabat, Chothia, EU, Martin (Enhanced Chothia), Contact, AbM, CCG, EU, or AHo, numbering method or a combination of two or more of theseIMGT, Martin (Enhanced Chothia), Contact, North, and AHo numbering methods, including as determined by a combination of any two or more of these numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software.

More specifically, the VH and VL for an antibody or antigen-binding fragment that binds RSV-F and/or MPV-F comprise or consist of any VH and any VL identified for anti-RSV/MPV MPK antibodies or MPH antibodies in Table 2 and Table 20, and have corresponding amino acid sequences set forth in Table 1 and the Sequence Listing. In some embodiments, the VH and VL are both from the same antibody identified in Table 2 and Table 20. However, in other embodiments, the VH may be from a first antibody identified in Table 2 or Table 20, while VL may be from a second, different antibody identified in Table 2 or Table 20. In some such instances, the first and second antibodies may both be MPK antibodies or the first and second antibodies may both the MPH antibodies. However, a VH from a MPK antibody and a VL from an MPH antibody and vice versa may also be used, particularly if V(D)J usage is the same between the MPH antibody and the MPK antibody.

In a specific embodiment, the CDRs for an antibody or antigen-binding fragment that binds RSV-F and/or MPV-F comprise or consist of the CDRs of MPK190-v1.3, MPK9, MPK10, MPK18, MPK30-v1, MPK36-v3, MPK51, MPK51-v1.1, MPK67, MPK73, MPK77, MPK77-v1.1, MPK86, MPK92, MPK99, MPK102, MPK104, MPK104-v1.1, MPK104, v1.3, MPK108, MPK126, MPK127, MPK129, MPK130, MPK132-v2, MPK133, MPK136, MPK141, MPK142-v1, MPK142-v2, MPK144, MPK145, MPK146, MPK149, MPK150-v2, MPK151, MPK152, MPK153, MPK155, MPK157, MPK158, MPK162, MPK174-v2, MPK190, MPK190-v1.1, MPK196, MPK204, MPH12, or MPH12 variants disclosed herein. In some embodiments, the CDRs are all from the same antibody identified in Table 2 and Table 20, such as all from MPK190-v1.3, MPK190-v1.1, MPK51-v1.1, MPK77-v1.1, MPK104-v1.1, or MPK 104-v1.3. However, in other embodiments, the CDRH1, CDRH2, and CDRH3 may be from a first antibody identified in Table 2 or Table 20, while the CDRL1, CDRL2, and CDRL3 may be from a second, different antibody identified in Table 2 or Table 20. In some such instances, the first and second antibodies may both be MPK antibodies or the first and second antibodies may both the MPH antibodies. However, a CDRH1, CDRH2, and CDRH3 from a MPK antibody and a CDRL1, CDRL2, and CDRL3 from an MPH antibody and vice versa may also be used, particularly if V(D)J usage is the same between the MPH antibody and the MPK antibody. In some embodiments, one or more of the first and second antibodies is MPK190-v1.3. In some embodiments, one or more of the first and second antibodies is MPK190-v1.1. In some embodiments, one or more of the first and second antibodies is MPK51-v1.1. In some embodiments, one or more of the first and second antibodies is MPK77-v1.1. In some embodiments, one or more of the first and second antibodies is MPK104-v1.1. In some embodiments, one or more of the first and second antibodies is MPK 104-v1.3.

Cross-binding and/or cross-neutralizing antibodies and antigen-binding fragments of the present disclosure may also be used to bind RSV-F alone, or to bind MPV-F alone; their utility is not limited to a context where binding and/or neutralization of both RSV and MPV targets occurs.

More specifically, the VH and VL for an antibody or antigen-binding fragment that binds RSV-F and/or MPV-F and/or neutralizes RSV and/or MPV comprise or consist of a VH and VL having the following sequences: 1) SEQ ID NOs.: 129 and 133; 2) SEQ ID NOs.: 12 and 17; 3) SEQ ID NOs.: 38 and 42; 4) SEQ ID NOs.: 46 and 50; 5) SEQ ID NOs.: 53 and 56; 6) SEQ ID NOs.: 59 and 64; 7) SEQ ID NOs.: 69 and 71; 8) SEQ ID NOs.: 120 and 125; 9) SEQ ID NOs.: 73 and 76; 10) SEQ ID NOs.: 79 and 83; 11) SEQ ID NOs.: 86 and 91; 12) SEQ ID NOs.: 95 and 98; 13) SEQ ID NOs.: 22 and 27; 14) SEQ ID NOs.: 111 and 116; 15) SEQ ID NOs.: 30 and 35; 16) SEQ ID NOs.: 378 and 382; 17) SEQ ID NOs.: 386 and 391; 18) SEQ ID NOs.: 395 and 400; 19) SEQ ID NOs.: 404 and 408; 20) SEQ ID NOs.: 412 and 414; 21) SEQ ID NOs.: 416 and 419; 22) SEQ ID NOs.: 422 and 424; 23) SEQ ID NOs.: 426 and 429; 24) SEQ ID NOs.: 431 and 434; 25) SEQ ID NOs.: 431 and 436; 26) SEQ ID NOs.: 440 and 444; 27) SEQ ID NOs.: 431 and 449; 28) SEQ ID NOs.: 451 and 455; 29) SEQ ID NOs.: 458 and 461; 30) SEQ ID NOs.: 463 and 466; 31) SEQ ID NOs.: 470 and 473; 32) SEQ ID NOs.: 475 and 478; 33) SEQ ID NOs.: 480 and 483; 34) SEQ ID NOs.: 485 and 487; 35) SEQ ID NOs.: 491 and 495; 36) SEQ ID NOs.: 497 and 501; 37) SEQ ID NOs.: 504 and 509; 38) SEQ ID NOs.: 513 and 518; 39) SEQ ID NOs.: 120 and 522; 40) SEQ ID NOs.: 524 and 518; 41) SEQ ID NOs.: 527 and 530; 42) SEQ ID NOs.: 532 and 553; 43) SEQ ID NOs.: 532 and 558; 44) SEQ ID NOs.: 532 and 577; 45) SEQ ID NOs.: 532 and 563; 46) SEQ ID NOs.: 532 and 567; 47) SEQ ID NOs.: 532 and 570; 48) SEQ ID NOs.: 532 and 572; 49) SEQ ID NOs.: 532 and 574; 50) SEQ ID NOs.: 537 and 553; 51) SEQ ID NOs.: 537 and 558; 52) SEQ ID NOs.: 537 and 577; 53) SEQ ID NOs.: 537 and 563; 54) SEQ ID NOs.: 537 and 567; 55) SEQ ID NOs.: 537 and 570; 56) SEQ ID NOs.: 537 and 572; 57) SEQ ID NOs.: 537 and 574; 58) SEQ ID NOs.: 539 and 553; 59) SEQ ID NOs.: 539 and 558; 60) SEQ ID NOs.: 539 and 577; 61) SEQ ID NOs.: 539 and 563; 62) SEQ ID NOs.: 539 and 567; 63) SEQ ID NOs.: 539 and 570; 64) SEQ ID NOs.: 539 and 572; 65) SEQ ID NOs.: 539 and 574; 66) SEQ ID NOs.: 542 and 553; 67) SEQ ID NOs.: 542 and 558; 68) SEQ ID NOs.: 542 and 577; 69) SEQ ID NOs.: 542 and 563; 70) SEQ ID NOs.: 542 and 567; 71) SEQ ID NOs.: 542 and 570; 72) SEQ ID NOs.: 542 and 572; 73) SEQ ID NOs.: 542 and 574; 74) SEQ ID NOs.: 545 and 553; 75) SEQ ID NOs.: 545 and 558; 76) SEQ ID NOs.: 545 and 577; 77) SEQ ID NOs.: 545 and 563; 78) SEQ ID NOs.: 545 and 567; 79) SEQ ID NOs.: 545 and 570; 80) SEQ ID NOs.: 545 and 572; 81) SEQ ID NOs.: 545 and 574; 82) SEQ ID NOs.: 547 and 553; 83) SEQ ID NOs.: 547 and 558; 84) SEQ ID NOs.: 547 and 577; 85) SEQ ID NOs.: 547 and 563; 86) SEQ ID NOs.: 547 and 567; 87) SEQ ID NOs.: 547 and 570; 88) SEQ ID NOs.: 547 and 572; 89) SEQ ID NOs.: 547 and 574; 90) SEQ ID NOs.: 550 and 553, 91) SEQ ID NOs.: 702 and 704; 92) SEQ ID NOs.: 707 and 708; 93) SEQ ID NOs.: 707 and 709; 94) SEQ ID NOs.: 712 and 76; 95) SEQ ID NOs.: 716 and 64; of 96) SEQ ID NOs.: 717 and 116, respectively.

In certain embodiments, the present disclosure provides an antibody or antigen-binding fragment that binds RSV-F, comprising a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are determined according to the IMGT numbering system, and wherein: (i) optionally, the CDRH1 comprises or consists of the amino acid sequence set forth in any one of SEQ ID NOs.: 3, 137, 147, 160, 170, 182, 216, 234, 244, 262, 278, 285, 301, 333, 363, 370, 13, 23, 31, 39, 54, 60, 74, 80, 87, 112, 121, 130, 380, 387, 396, 728, 738, 747, 756, 766, 776, 785, 795, 805, 814, 887, 890, 417, 441, 452, 459, 471, 481, 492, 498, 505, 514, 528, and 533 or a functional variant thereof comprising one, two, or three acid substitutions, one or more of which substitutions is optionally a conservative substitution and/or is a substitution to a germline-encoded amino acid; (ii) optionally, the CDRH2 comprises or consists of the amino acid sequence set forth in any one of SEQ ID NOs.: 4, 138, 161, 217, 226, 235, 263, 279, 302, 334, 364, 371, 729, 739, 748, 757, 767, 777, 786, 796, 815, 818, 838, 884, 894, 897, 14, 24, 32, 40, 47, 61, 81, 88, 96, 113, 122, 388, 397, 405, 442, 453, 464, 506, 515, 534, 540, 543, and 548 or a functional variant thereof comprising one, two, or three amino acid substitutions, one or more of which substitutions is optionally a conservative substitution and/or is a substitution to a germline-encoded amino acid; (iii) optionally, the CDRH3 comprises or consists of the amino acid sequence set forth in any one of SEQ ID NOs.: 5, 139, 148, 162, 176, 183, 190, 197, 203, 218, 227, 236, 245, 255, 264, 272, 280, 286, 294, 303, 310, 317, 322, 328, 335, 343, 353, 358, 365, 372, 730, 740, 749, 758, 768, 778, 787, 797, 806, 821, 824, 832, 835, 841, 844, 15, 25, 33, 48, 62, 89, 114, 123, 131, 389, 398, 406, 427, 432, 476, 493, 499, 507, 516, 535, and 551 or a functional variant thereof comprising one, two, or three amino acid substitutions, one or more of which substitutions is optionally a conservative substitution and/or is a substitution to a germline-encoded amino acid; (iv) optionally, the CDRL1 comprises or consists of the amino acid sequence set forth in any one of SEQ ID NOs.: 8, 142, 151, 165, 173, 186, 193, 206, 221, 230, 239, 258, 267, 289, 297, 306, 313, 338, 350, 375, 733, 743, 761, 771, 781, 790, 800, 809, 18, 65, 92, 99, 126, 383, 401, 437, 445, 456, 488, 554, 561, 564, and 568 or a functional variant thereof comprising one, two, or three amino acid substitutions, one or more of which substitutions is optionally a conservative substitution and/or is a substitution to a germline-encoded amino acid; (v) optionally, the CDRL2 comprises or consists of the amino acid sequence set forth in any one of SEQ ID NOs.: 9, 143, 152, 156, 166, 200, 207, 222, 240, 268, 290, 314, 339, 734, 752, 762, 772, 791, 801, 856, 871, 19, 43, 66, 117, 392, 409, 467, 489, 510, 519, 555, and 705, or a functional variant thereof comprising one, two, or three amino acid substitutions, one or more of which substitutions is optionally a conservative substitution and/or is a substitution to a germline-encoded amino acid; and/or (vi) optionally, the CDRL3 comprises or consists of the amino acid sequence set forth in any one of SEQ ID NOs.: 10, 144, 153, 157, 167, 179, 187, 194, 208, 213, 223, 231, 241, 248, 259, 269, 275, 291, 298, 307, 325, 340, 376, 735, 744, 753, 763, 773, 782, 792, 802, 810, 848, 852, 866, 20, 28, 36, 44, 51, 57, 67, 77, 84, 93, 118, 127, 134, 384, 393, 402, 410, 420, 438, 446, 468, 502, 511, 556, and 520, or a functional variant thereof comprising having one, two, or three amino acid substitutions, one or more of which substitutions is optionally a conservative substitution and/or is a substitution to a germline-encoded amino acid.

In certain embodiments, the present disclosure provides an antibody or antigen-binding fragment that binds RSV-F, comprising a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are determined according to the IMGT numbering system, and wherein: (i) optionally, the CDRH1 comprises or consists of the amino acid sequence set forth in any one of SEQ ID NOs.: 3, 137, 147, 160, 170, 182, 216, 234, 244, 262, 278, 285, 301, 333, 363, 370, 728, 738, 747, 756, 766, 776, 785, 795, 805, 814, 887, and 890 or a functional variant thereof comprising one, two, or three acid substitutions, one or more of which substitutions is optionally a conservative substitution and/or is a substitution to a germline-encoded amino acid; (ii) optionally, the CDRH2 comprises or consists of the amino acid sequence set forth in any one of SEQ ID NOs.: 4, 138, 161, 217, 226, 235, 263, 279, 302, 334, 364, 371, 729, 739, 748, 757, 767, 777, 786, 796, 815, 818, 838, 884, 894, and 897 or a functional variant thereof comprising one, two, or three amino acid substitutions, one or more of which substitutions is optionally a conservative substitution and/or is a substitution to a germline-encoded amino acid; (iii) optionally, the CDRH3 comprises or consists of the amino acid sequence set forth in any one of SEQ ID NOs.: 5, 139, 148, 162, 176, 183, 190, 197, 203, 218, 227, 236, 245, 255, 264, 272, 280, 286, 294, 303, 310, 317, 322, 328, 335, 343, 353, 358, 365, 372, 730, 740, 749, 758, 768, 778, 787, 797, 806, 821, 824, 832, 835, 841, and 844 or a functional variant thereof comprising one, two, or three amino acid substitutions, one or more of which substitutions is optionally a conservative substitution and/or is a substitution to a germline-encoded amino acid; (iv) optionally, the CDRL1 comprises or consists of the amino acid sequence set forth in any one of SEQ ID NOs.: 8, 142, 151, 165, 173, 186, 193, 206, 221, 230, 239, 258, 267, 289, 297, 306, 313, 338, 350, 375, 733, 743, 761, 771, 781, 790, 800, and 809 or a functional variant thereof comprising one, two, or three amino acid substitutions, one or more of which substitutions is optionally a conservative substitution and/or is a substitution to a germline-encoded amino acid; (v) optionally, the CDRL2 comprises or consists of the amino acid sequence set forth in any one of SEQ ID NOs.: 9, 143, 152, 156, 166, 200, 207, 222, 240, 268, 290, 314, 339, 734, 752, 762, 772, 791, 801, 856, and 871 or a functional variant thereof comprising one, two, or three amino acid substitutions, one or more of which substitutions is optionally a conservative substitution and/or is a substitution to a germline-encoded amino acid; and/or (vi) optionally, the CDRL3 comprises or consists of the amino acid sequence set forth in any one of SEQ ID NOs.: 10, 144, 153, 157, 167, 179, 187, 194, 208, 213, 223, 231, 241, 248, 259, 269, 275, 291, 298, 307, 325, 340, 376, 735, 744, 753, 763, 773, 782, 792, 802, 810, 848, 852, and 866 or a functional variant thereof comprising having one, two, or three amino acid substitutions, one or more of which substitutions is optionally a conservative substitution and/or is a substitution to a germline-encoded amino acid.

In further embodiments, CDRH1, CDRH2, CDRH3, CDRL1, CDRL2, and CDRL3 for an antibody or antigen-binding fragment that binds RSV-F comprise or consist of the CDRs identified for anti-RSV MPK antibodies in Table 2, and have corresponding amino acid sequences set forth in Table 1 and the Sequence Listing. Specifically, the CDRs can comprise or consist of the CDRs of MPK44, MPK65-v2, MPK161-v2, MPK163, MPK165, MPK167, MPK168, MPK169-v2, MPK170, MPK171-v1, MPK171-v2, MPK173, MPK175, MPK176, MPK177, MPK178, MPK179-v4, MPK180, MPK181, MPK182, MPK185, MPK186, MPK187, MPK188, MPK189, MPK191, MPK193, MPK194-v2, MPK195, MPK197, MPK198, MPK201, MPK202, MPK203, MPM10, MPM2, MPM8, MPO1, MPO7, MPP1, MPP2, MPR16, or MPR19-v2, MPK65-v (any one of 1-7).(any one 1-2), MPK201-v (any one of 1-6).(any one of 1-2), MPK176-v (any one of 1-6).(any one of 5), or MPM2-v (any one of 1, 2, 4, 5).(any one of 1-9), specifically MPK176-v1.3, MPK176-v4.3, MPK201-v1.1, MPK201-v1.2, MPK201-v4.1, MPK65v2-v1.2, or MPK65v2-v3.1.

More specifically, the CDRH1, CDRH2, CDRH3, CDRL1, CDRL2, and CDRL3 for an antibody or antigen-binding fragment that binds RSV-F comprise or consist of CDRs having the following sequences: 1) SEQ ID NOs.: 3-5 and 8-10; 2) SEQ ID NOs.: 137-139 and 142-144; 3) SEQ ID NOs.: 147, 138, 148, and 151-153; 4) SEQ ID NOs.: 147, 138, 148, 142, 156, and 157; 5) SEQ ID NOs.: 160-162 and 165-167; 6) SEQ ID NOs.: 170, 138, 148, 173, 156, and 157; 7) SEQ ID NOs.: 137, 138, 176, 142, 143, and 179; 8) SEQ ID NOs.: 182, 138, 183, 186, 143, and 187; 9) SEQ ID NOs.: 137, 138, 190, 193, 143, 194; 10) SEQ ID NOs.: 137, 138, 197, 142, 200, and 157; 11) SEQ ID NOs.: 137, 138, 203, and 206-208; 12) SEQ ID NOs.: 137, 138, 203, 142, 143, and 213; 13) SEQ ID NOs.: 216-218 and 221-223; 14) SEQ ID NOs.: 137, 226, 227,230, 143, and 231; 15) SEQ ID NOs.: 234-236 and 239-241; 16) SEQ ID NOs.: 244, 138, 245, 173, 143, and 248; 17) SEQ ID NOs.: 182, 138, 148, 142, 156, and 157; 18) SEQ ID NOs.: 137, 138, 255, 258, 156, and 259; 19) SEQ ID NOs.: 262-264 and 267-269; 20) SEQ ID NOs.: 137, 138, 272, 173, 143, and 275; 21) SEQ ID NOs.: 278-280 and 206-208; 22) SEQ ID NOs.: 285, 138, 286, and 289-291; 23) SEQ ID NOs.: 137, 226, 294, 297, 143, and 298; 24) SEQ ID NOs.: 301-303, 306, 268, and 307; 25) SEQ ID NOs.: 137, 138, 310, 313, 314, and 157; 26) 137,138, 317, 142, 143, and 213; 27) SEQ ID NOs.: 137, 138, 322, 142, 143, and 325; 28) SEQ ID NOs.: 137, 138, 322, 142,143, and 325; 29) SEQ ID NOs.: 170, 138, 328, 142, 143, and 157; 30) SEQ ID NOs.: 333-335 and 338-340; 31) SEQ ID NOs.: 137, 138, 343, 173, 143, and 213; 32) SEQ ID NOs.: 301-303, 350, 268, and 307; 33) SEQ ID NOs.: 137, 138, 353, 142, 143, and 213; 34) SEQ ID NOs.: 137, 226, 358, 142, 143, and 231; 35) SEQ ID NOs.: 363-365 and 8-10; 36) SEQ ID NOs.: 370-372, 375, 143, and 376; 37) SEQ ID NOs.: 738, 884, 740, 743, 240, and 744; 38) SEQ ID NOs.: 738-740, 743, 240, and 744; 39) SEQ ID NOs.: 728-730, and 733-735; 40) SEQ ID NOs.: 747-749, 221, and 752-753; 41) SEQ ID NOs.: 756-758, and 761-763; 42) SEQ ID NOs.: 766-768, and 771-773; 43) SEQ ID NOs.: 776-778, 781, 268, and 782; 44) SEQ ID NOs.: 785-787, and 790-792; 45) SEQ ID NOs.: 795-797, and 800-802; 46) SEQ ID NOs.: 805, 796, 806, 809, 801, and 810; 47) SEQ ID NOs.: 234, 838, 236, and 239-241; 48) SEQ ID NOs.: 137, 226, 358, 142, 143, and 848; 49) SEQ ID NOs.: 814, 226, 358, 142, 143, and 231; 50) SEQ ID NOs.: 137-139, 142, 143, and 852; 51) SEQ ID NOs.: 814, 818, 139 and 142-144; 52) 738, 884, 740, 743, 240, and 744; 53) SEQ ID NOs.: 887, 884, 740, 743, 240, and 744; or 54) SEQ ID NOs.: 738, 884, 740, 743, 240, and 744, respectively.

In further embodiments, CDRH1, CDRH2, CDRH3, CDRL1, CDRL2, and CDRL3 for an antibody or antigen-binding fragment that binds MPV-F comprise or consist of the CDRs identified for anti-MPV MPK antibodies in Table 2, and have corresponding amino acid sequences set forth in Table 1 and the Sequence Listing. Specifically, the CDRs comprise or consist of the CDRs of MPK15 disclosed herein.

In certain embodiments, the present disclosure provides an antibody or antigen-binding fragment that binds MPV-F, comprising a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are determined according to the IMGT numbering system, and wherein: (i) optionally, the CDRH1 comprises or consists of the amino acid sequence set forth in any one of SEQ ID NOs.: 102, 13, 23, 31, 39, 54, 60, 74, 80, 87, 112, 121, 130, 380, 387, 396, 417, 441, 452, 459, 471, 481, 492, 498, 505, 514, 528, and 533 or a functional variant thereof comprising one, two, or three acid substitutions, one or more of which substitutions is optionally a conservative substitution and/or is a substitution to a germline-encoded amino acid; (ii) optionally, the CDRH2 comprises or consists of the amino acid sequence set forth in any one of SEQ ID NOs.: 103, 14, 24, 32, 40, 47, 61, 81, 88, 96, 113, 122, 388, 397, 405, 442, 453, 464, 506, 515, 534, 540, 543, and 548 or a functional variant thereof comprising one, two, or three amino acid substitutions, one or more of which substitutions is optionally a conservative substitution and/or is a substitution to a germline-encoded amino acid; (iii) optionally, the CDRH3 comprises or consists of the amino acid sequence set forth in any one of SEQ ID NO.: 104, 15, 25, 33, 48, 62, 89, 114, 123, 131, 389, 398, 406, 427, 432, 476, 493, 499, 507, 516, 535, and 551 or a functional variant thereof comprising one, two, or three amino acid substitutions, one or more of which substitutions is optionally a conservative substitution and/or is a substitution to a germline-encoded amino acid; (iv) optionally, the CDRL1 comprises or consists of the amino acid sequence set forth in any one of SEQ ID NOs.: 107, 18, 65, 92, 99, 126, 383, 401, 437, 445, 456, 488, 554, 561, 564, and 568 or a functional variant thereof comprising one, two, or three amino acid substitutions, one or more of which substitutions is optionally a conservative substitution and/or is a substitution to a germline-encoded amino acid; (v) optionally, the CDRL2 comprises or consists of the amino acid sequence set forth in any one of SEQ ID NOs.: 108, 19, 43, 66, 117, 392, 409, 467, 489, 510, 519, 555, and 705, or a functional variant thereof comprising one, two, or three amino acid substitutions, one or more of which substitutions is optionally a conservative substitution and/or is a substitution to a germline-encoded amino acid; and/or (vi) optionally, the CDRL3 comprises or consists of the amino acid sequence set forth in any one of SEQ ID NOs.: 109, 20, 28, 36, 44, 51, 57, 67, 77, 84, 93, 118, 127, 134, 384, 393, 402, 410, 420, 438, 446, 468, 502, 511, 556, and 520, or a functional variant thereof comprising having one, two, or three amino acid substitutions, one or more of which substitutions is optionally a conservative substitution and/or is a substitution to a germline-encoded amino acid.

In certain embodiments, the present disclosure provides an antibody or antigen-binding fragment that binds MPV-F, comprising a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are determined according to the IMGT numbering system, and wherein: (i) optionally, the CDRH1 comprises or consists of the amino acid sequence set forth SEQ ID NO.: 102, or a functional variant thereof comprising one, two, or three acid substitutions, one or more of which substitutions is optionally a conservative substitution and/or is a substitution to a germline-encoded amino acid; (ii) optionally, the CDRH2 comprises or consists of the amino acid sequence set forth in SEQ ID NO.: 103, or a functional variant thereof comprising one, two, or three amino acid substitutions, one or more of which substitutions is optionally a conservative substitution and/or is a substitution to a germline-encoded amino acid; (iii) optionally, the CDRH3 comprises or consists of the amino acid sequence set forth in SEQ ID NO.: 104, or a functional variant thereof comprising one, two, or three amino acid substitutions, one or more of which substitutions is optionally a conservative substitution and/or is a substitution to a germline-encoded amino acid; (iv) optionally, the CDRL1 comprises or consists of the amino acid sequence set forth in SEQ ID NOs.: 107, or a functional variant thereof comprising one, two, or three amino acid substitutions, one or more of which substitutions is optionally a conservative substitution and/or is a substitution to a germline-encoded amino acid; (v) optionally, the CDRL2 comprises or consists of the amino acid sequence set forth SEQ ID NOs.: 108, or a functional variant thereof comprising one, two, or three amino acid substitutions, one or more of which substitutions is optionally a conservative substitution and/or is a substitution to a germline-encoded amino acid; and/or (vi) optionally, the CDRL3 comprises or consists of the amino acid sequence set forth in SEQ ID NOs.: 109, or a functional variant thereof comprising having one, two, or three amino acid substitutions, one or more of which substitutions is optionally a conservative substitution and/or is a substitution to a germline-encoded amino acid.

In certain embodiments, the present disclosure provides an antibody or antigen-binding fragment that binds RSV-F and/or MPV-F, comprising a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are determined according to the IMGT numbering system, and wherein: (i) optionally, the CDRH1 comprises or consists of the amino acid sequence set forth in any one of SEQ ID NOs.: 13, 23, 31, 39, 54, 60, 74, 80, 87, 112, 121, 130, 380, 387, 396, 417, 441, 452, 459, 471, 481, 492, 498, 505, 514, 528, and 533 or a functional variant thereof comprising one, two, or three acid substitutions, one or more of which substitutions is optionally a conservative substitution and/or is a substitution to a germline-encoded amino acid; (ii) optionally, the CDRH2 comprises or consists of the amino acid sequence set forth in any one of SEQ ID NOs.: 14, 24, 32, 40, 47, 61, 81, 88, 96, 113, 122, 388, 397, 405, 442, 453, 464, 506, 515, 534, 540, 543, and 548 or a functional variant thereof comprising one, two, or three amino acid substitutions, one or more of which substitutions is optionally a conservative substitution and/or is a substitution to a germline-encoded amino acid; (iii) optionally, the CDRH3 comprises or consists of the amino acid sequence set forth in any one of SEQ ID NOs.: 15, 25, 33, 48, 62, 89, 114, 123, 131, 389, 398, 406, 427, 432, 476, 493, 499, 507, 516, 535, and 551 or a functional variant thereof comprising one, two, or three amino acid substitutions, one or more of which substitutions is optionally a conservative substitution and/or is a substitution to a germline-encoded amino acid; (iv) optionally, the CDRL1 comprises or consists of the amino acid sequence set forth in any one of SEQ ID NOs.: 18, 65, 92, 99, 126, 383, 401, 437, 445, 456, 488, 554, 561, 564, and 568 or a functional variant thereof comprising one, two, or three amino acid substitutions, one or more of which substitutions is optionally a conservative substitution and/or is a substitution to a germline-encoded amino acid; (v) optionally, the CDRL2 comprises or consists of the amino acid sequence set forth in any one of SEQ ID NOs.: 19, 43, 66, 117, 392, 409, 467, 489, 510, 519, 555, and 705, or a functional variant thereof comprising one, two, or three amino acid substitutions, one or more of which substitutions is optionally a conservative substitution and/or is a substitution to a germline-encoded amino acid; and/or (vi) optionally, the CDRL3 comprises or consists of the amino acid sequence set forth in any one of SEQ ID NOs.: 20, 28, 36, 44, 51, 57, 67, 77, 84, 93, 118, 127, 134, 384, 393, 402, 410, 420, 438, 446, 468, 502, 511, 556, and 520, or a functional variant thereof comprising having one, two, or three amino acid substitutions, one or more of which substitutions is optionally a conservative substitution and/or is a substitution to a germline-encoded amino acid.

In further embodiments, CDRH1, CDRH2, CDRH3, CDRL1, CDRL2, and CDRL3 for an antibody or antigen-binding fragment that binds RSV-F and/or MPV-F comprise or consist of the CDRs identified for anti-RSV/MPV MPK antibodies or MPH antibodies in Table 2 and Table 20, and have corresponding amino acid sequences set forth in Table 1 and the Sequence Listing. Specifically, the CDRs comprise or consist of the CDRs of MPK190-v1.3, MPK9, MPK10, MPK18, MPK30-v1, MPK36-v3, MPK51, MPK51-v1.1, MPK67, MPK73, MPK77, MPK77-v1.1, MPK86, MPK92, MPK99, MPK102, MPK104, MPK104-v.1.1, MPK104-v1.3, MPK108, MPK126, MPK127, MPK129, MPK130, MPK132-v2, MPK133, MPK136, MPK141, MPK142-v1, MPK142-v2, MPK144, MPK145, MPK146, MPK149, MPK150-v2, MPK151, MPK152, MPK153, MPK155, MPK157, MPK158, MPK162, MPK174-v2, MPK190, MPK190-v1.1, MPK196, MPK204, MPH12, and variants of MPH12 disclosed herein. These cross-binding or cross-neutralizing antibodies may also be used to bind RSV-F alone, or to bind MPV-F alone; they need not be used in a context where binding and/or neutralization of both RSV and MPV targets occurs.

The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype, into CHi, CH2, and CH3 (IgA, IgD, IgG), or CHi, CH2, CH3, and CH4 domains (IgE, IgM). The Fc region of an antibody heavy chain is described further herein. In any of the presently disclosed embodiments, an antibody or antigen-binding fragment of the present disclosure comprises any one or more of CL, a CHi, a CH2, and a CH3. In any of the presently disclosed embodiments, an antibody or antigen-binding fragment of the present disclosure may comprise any one or more of CL, a CHi, a CH2, and a CH3. In certain embodiments, a CL comprises an amino acid sequence having at least 90%, at least 91%, at least 92%, at least 93%, at least 94%, at least 95%, at least 96%, at least 97%, at least 98%, at least 99%, or 100% identity to the amino acid sequence of SEQ ID NO.:905. In certain embodiments, a CL comprises an amino acid sequence having at least 90%, at least 91%, at least 92%, at least 93%, at least 94%, at least 95%, at least 96%, at least 97%, at least 98%, at least 99%, or 100% identity to a human lambda light chain constant domain.

In certain embodiments, a CH1-CH3 comprises an amino acid sequence having at least 90%, at least 91%, at least 92%, at least 93%, at least 94%, at least 95%, at least 96%, at least 97%, at least 98%, at least 99%, or 100% identity to the amino acid sequence of any one of SEQ ID NOs.:664-668.

It will be understood that, for example, production in a mammalian cell line can remove one or more C-terminal lysine of an antibody heavy chain (see, e.g., Liu et al. mAbs 6(5):1145-1154 (2014)). Accordingly, an antibody or antigen-binding fragment of the present disclosure can comprise a heavy chain, a CH1-CH3, a CH3, or an Fc polypeptide wherein a C-terminal lysine residue or a C-terminal glycine-lysine is present or is absent; in other words, encompassed are embodiments where the C-terminal residue of a heavy chain, a CH1-CH3, or an Fc polypeptide is not a lysine, and embodiments where a lysine is the C-terminal residue. In certain embodiments, a composition comprises a plurality of an antibody and/or an antigen-binding fragment of the present disclosure, wherein one or more antibody or antigen-binding fragment does not comprise a lysine residue or a C-terminal glycine-lysine at the C-terminal end of the heavy chain, CH1-CH3, or Fc polypeptide, and wherein one or more antibody or antigen-binding fragment comprises a lysine residue at the C-terminal end of the heavy chain, CH1-CH3, or Fc polypeptide.

A “Fab” (fragment antigen binding) is the part of an antibody that binds to antigens and includes the variable region and CHi of the heavy chain linked to the light chain via an inter-chain disulfide bond. Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and still cross-links antigen. Both the Fab and F(ab′)2 are examples of “antigen-binding fragments.” Fab′ fragments differ from Fab fragments by having additional few residues at the carboxy terminus of the CHi domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them.

Other chemical couplings of antibody fragments are also known.

Fab fragments may be joined, e.g., by a peptide linker, to form a single chain Fab, also referred to herein as “scFab.” In these embodiments, an inter-chain disulfide bond that is present in a native Fab may not be present, and the linker serves in full or in part to link or connect the Fab fragments in a single polypeptide chain. A heavy chain-derived Fab fragment (e.g., comprising, consisting of, or consisting essentially of VH+CH1, or “Fd”) and a light chain-derived Fab fragment (e.g., comprising, consisting of, or consisting essentially of VL+CL) may be linked in any arrangement to form a scFab. For example, a scFab may be arranged, in N-terminal to C-terminal direction, according to (heavy chain Fab fragment-linker-light chain Fab fragment) or (light chain Fab fragment-linker-heavy chain Fab fragment). Peptide linkers and exemplary linker sequences for use in scFabs are discussed in further detail herein. “Fv” is a small antibody fragment that contains a complete antigen-recognition and antigen-binding site. This fragment generally consists of a dimer of one heavy- and one light-chain variable region domain in tight, non-covalent association. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv”, are antibody fragments that comprise the VH and V_{L} antibody domains connected into a single polypeptide chain. In some embodiments, the scFv polypeptide comprises a polypeptide linker disposed between and linking the VH and V_{L} domains that enables the scFv to retain or form the desired structure for antigen binding. Such a peptide linker can be incorporated into a fusion polypeptide using standard techniques well known in the art. For a review of scFv, see Pluckthun in The Pharmacology of Monoclonal Antibodies, vol. 113, Rosenburg and Moore eds., Springer-Verlag, New York, pp. 269-315 (1994); Borrebaeck 1995, infra. In certain embodiments, the antibody or antigen-binding fragment comprises a scFv comprising a VH domain, a VL domain, and a peptide linker linking the VH domain to the VL domain. In particular embodiments, a scFv comprises a VH domain linked to a VL domain by a peptide linker, which can be in a VH-linker VL orientation or in a VL-linker VH orientation. Any scFv of the present disclosure may be engineered so that the C-terminal end of the VL domain is linked by a short peptide sequence to the N-terminal end of the VH domain, or vice versa (i.e., (N)VL(C)-linker-(N)VH(C) or (N)VH(C)-linker-(N)VL(C). Alternatively, in some embodiments, a linker may be linked to an N-terminal portion or end of the VH domain, the VL domain, or both.

Peptide linker sequences may be chosen, for example, based on: (1) their ability to adopt a flexible extended conformation; (2) their inability or lack of ability to adopt a secondary structure that could interact with functional epitopes on the first and second polypeptides and/or on a target molecule; and/or (3) the lack or relative lack of hydrophobic or charged residues that might react with the polypeptides and/or target molecule. Other considerations regarding linker design (e.g., length) can include the conformation or range of conformations in which the VH and VL can form a functional antigen-binding site. In certain embodiments, peptide linker sequences contain, for example, Gly, Asn and Ser residues. Other near neutral amino acids, such as Thr and Ala, may also be included in a linker sequence. Other amino acid sequences which may be usefully employed as linker include those disclosed in Maratea et al., Gene 40:39 46 (1985); Murphy et al., Proc. Natl. Acad. Sci. USA 83:8258 8262 (1986); U.S. Pat. Nos. 4,935,233, and 4,751,180. Other illustrative and non-limiting examples of linkers may include, for example, Glu-Gly-Lys-Ser-Ser-Gly-Ser-Gly-Ser-Glu-Ser-Lys-Val-Asp (Chaudhary et al., Proc. Natl. Acad. Sci. USA 87:1066-1070 (1990)) and Lys-Glu-Ser-Gly-Ser-Val-Ser-Ser-Glu-Gln-Leu-Ala-Gln-Phe-Arg-Ser-Leu-Asp (Bird et al., Science 242:423-426 (1988)) and the pentamer Gly-Gly-Gly-Gly-Ser when present in a single iteration or repeated 1 to 5 or more times, or more. Any suitable linker may be used, and in general can be about 3, 4, 5, 6, 7, 8, 9, 10, 11, 12, 13, 14, 15, 16, 17, 18, 19, 20, 21, 22, 15 23, 24, 25, 26, 27, 28, 29, 30, 40, 50, 60, 70, 80, 90, 100 amino acids in length, or less than about 200 amino acids in length, and will preferably comprise a flexible structure (can provide flexibility and room for conformational movement between two regions, domains, motifs, fragments, or modules connected by the linker), and will preferably be biologically inert and/or have a low risk of immunogenicity in a human.

scFvs can be constructed using any combination of the VH and VL sequences or any combination of the CDRH1, CDRH2, CDRH3, CDRL1, CDRL2, and CDRL3 sequences disclosed herein.

In some embodiments, linker sequences are not required; for example, when the first and second polypeptides have non-essential N-terminal amino acid regions that can be used to separate the functional domains and prevent steric interference.

During antibody development, DNA in the germline variable (V), joining (J), and diversity (D) gene loci may be rearranged and insertions and/or deletions of nucleotides in the coding sequence may occur. Somatic mutations may be encoded by the resultant sequence, and can be identified by reference to a corresponding known germline sequence. In some contexts, somatic mutations that are not critical to a desired property of the antibody (e.g., binding to a RSV and/or MPV fusion glycoprotein antigen), or that confer an undesirable property upon the antibody (e.g., an increased risk of immunogenicity in a subject administered the antibody), or both, may be replaced by the corresponding germline-encoded amino acid, or by a different amino acid, so that a desirable property of the antibody is improved or maintained and the undesirable property of the antibody is reduced or abrogated. Thus, in some embodiments, the antibody or antigen-binding fragment of the present disclosure comprises one or more more germline-encoded amino acid in a variable region as compared to a parent antibody or antigen-binding fragment, provided that the parent antibody or antigen binding fragment comprises one or more somatic mutations. Variable region and CDR amino acid sequences of exemplary anti-RSV-F, anti-MPV-F, and anti-RSV-F and/or MPV-F antibodies of the present disclosure are provided in Table 1, Table 2, and the Sequence Listing.

In certain embodiments, an antibody or antigen-binding fragment comprises an amino acid modification (e.g., a substitution mutation) to remove an undesired risk of oxidation, deamidation, and/or isomerization.

Also provided herein are variant antibodies that comprise one or more amino acid alterations in a variable region (e.g., VH, VL, framework or CDR) as compared to a presently disclosed (“parent”) antibody, wherein the variant antibody binds to a RSV and/or MPV fusion glycoprotein.

In certain embodiments, the antibody binds to RSV-F and
(i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 2, 136, 146, 159, 169, 175, 181, 189, 196, 202, 210, 215, 225, 233, 243, 250, 254, 261, 271, 277, 284, 293, 300, 309, 316, 321, 327, 332, 342, 347, 352, 357, 362, 369, 727, 737, 746, 755, 765, 775, 784, 794, 804, 813, 817, 820, 823, 826, 828, 831, 834, 837, 840, 843, 883, 886, 889, 893, 896, 899, 901, 903, 129, 12, 38, 46, 53, 59, 69, 120, 73, 79, 86, 95, 22, 111,30,378,386,395,404,412,416,422,426,431, 440, 451, 458, 463, 470, 475, 480, 485, 491, 497, 532, 537, 539, 542, 545, 547, 550, 504, 513, 524, 527, 702, 707, 712, and 716, wherein sequence variation is optionally limited to one or more framework regions and/or sequence variation comprises one or more substitution to a germline-encoded amino acid; and/or
(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 7, 141, 150, 155, 164, 172, 178, 185, 192, 199, 205, 212, 220, 229, 238, 247, 252, 257, 266, 274, 282, 288, 296, 305, 312, 319, 324, 330, 337, 345, 349, 355, 360, 367, 374, 732, 742, 751, 760, 770, 780, 789, 799, 808, 847, 851, 855, 858, 860, 862, 865, 868, 870, 873, 875, 877, 879, 881, 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 553, 558, 560, 563, 567, 570, 572, 574, 704, and 710, wherein sequence variation is optionally limited to one or more framework regions and/or sequence variation comprises one or more substitution to a germline-encoded amino acid.

In certain embodiments, the antibody binds to RSV-F and
(i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 2, 136, 146, 159, 169, 175, 181, 189, 196, 202, 210, 215, 225, 233, 243, 250, 254, 261, 271, 277, 284, 293, 300, 309, 316, 321, 327, 332, 342, 347, 352, 357, 362, 369, 727, 737, 746, 755, 765, 775, 784, 794, 804, 813, 817, 820, 823, 826, 828, 831, 834, 837, 840, 843, 883, 886, 889, 893, 896, 899, 901, and 903 wherein sequence variation is optionally limited to one or more framework regions and/or sequence variation comprises one or more substitution to a germline-encoded amino acid; and/or
(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 7, 141, 150, 155, 164, 172, 178, 185, 192, 199, 205, 212, 220, 229, 238, 247, 252, 257, 266, 274, 282, 288, 296, 305, 312, 319, 324, 330, 337, 345, 349, 355, 360, 367, 374, 732, 742, 751, 760, 770, 780, 789, 799, 808, 847, 851, 855, 858, 860, 862, 865, 868, 870, 873, 875, 877, 879, and 881 wherein sequence variation is optionally limited to one or more framework regions and/or sequence variation comprises one or more substitution to a germline-encoded amino acid.

In certain embodiments, the antibody binds to MVP-F and
(i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 101, 132, 16, 41, 49, 55, 63, 70, 124, 75, 82, 90, 97, 26, 115, 34, 381, 390, 399, 407, 413, 418, 423, 428, 433, 435, 443, 448, 454, 460, 465, 472, 477, 482, 486, 494, 500, 508, 517, 521, 525, 529, 703, 707, 712, and 716, wherein sequence variation is optionally limited to one or more framework regions and/or sequence variation comprises one or more substitution to a germline-encoded amino acid; and/or
(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 106, 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 553, 558, 560, 563, 567, 570, 572, 574, 704, and 710, wherein sequence variation is optionally limited to one or more framework regions and/or sequence variation comprises one or more substitution to a germline-encoded amino acid.

In certain embodiments, the antibody binds to MVP-F and
(i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in SEQ ID NO.: 101, wherein sequence variation is optionally limited to one or more framework regions and/or sequence variation comprises one or more substitution to a germline-encoded amino acid; and/or
(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in SEQ ID NO.: 106, wherein sequence variation is optionally limited to one or more framework regions and/or sequence variation comprises one or more substitution to a germline-encoded amino acid.

In certain embodiments, the antibody binds to RSV-F and/or MPV-F and
(i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 129, 12, 38, 46, 53, 59, 69, 120, 73, 79, 86, 95, 22, 111,30,378,386,395,404,412,416,422,426,431,440,451,458,463, 470, 475, 480, 485, 491, 497, 532, 537, 539, 542, 545, 547, 550, 504, 513, 524, 527, 702, 707, 712, and 716, wherein sequence variation is optionally limited to one or more framework regions and/or sequence variation comprises one or more substitution to a germline-encoded amino acid; and/or
(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 553, 558, 560, 563, 567, 570, 572, 574, 704, and 710, wherein sequence variation is optionally limited to one or more framework regions and/or sequence variation comprises one or more substitution to a germline-encoded amino acid.

In certain embodiments, the antibody binds to RSV-F and/or MPV-F and
(i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 532, 537, 539, 542, 545, 547, or 550, wherein sequence variation is optionally limited to one or more framework regions and/or sequence variation comprises one or more substitution to a germline-encoded amino acid; and/or
(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 553, 558, 560, 563, 567, 570, 572, or 574, wherein sequence variation is optionally limited to one or more framework regions and/or sequence variation comprises one or more substitution to a germline-encoded amino acid.

In certain embodiments, an antibody or antigen-binding fragment of the present disclosure is monospecific (e.g., binds to a single epitope) or is multispecific (e.g., binds to multiple epitopes and/or target molecules). Antibodies and antigen binding fragments may be constructed in various formats. Exemplary antibody formats disclosed in Spiess et al., Mol.

Immunol. 67(2):95 (2015), and in Brinkmann and Kontermann, mAbs 9(2):282-212 (2017), which formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, d-bodies, orthogonal Fabs, DVD-Igs (e.g., U.S. Pat. No. 8,258,268, which formats are incorporated herein by reference in their entirety), IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, and DVI-IgG (four-in-one), as well as so-called FIT-Ig (e.g., PCT Publication No. WO 2015/103072, which formats are incorporated herein by reference in their entirety), so-called WuxiBody formats (e.g., PCT Publication No. WO 2019/057122, which formats are incorporated herein by reference in their entirety), and so-called In-Elbow-Insert Ig formats (IEI-Ig; e.g., PCT Publication Nos. WO 2019/024979 and WO 2019/025391, which formats are incorporated herein by reference in their entirety).

In certain embodiments, the antibody or antigen-binding fragment comprises two or more of VH domains, two or more VL domains, or both (i.e., two or more VH domains and two or more VL domains). In particular embodiments, an antigen-binding fragment comprises the format (N-terminal to C-terminal direction) VH-linker VL-linker VH-linker VL, wherein the two VH sequences can be the same or different and the two VL sequences can be the same or different. Such linked scFvs can include any combination of VH and VL domains arranged to bind to a given target, and in formats comprising two or more VH and/or two or more VL, one, two, or more different epitopes or antigens may be bound. It will be appreciated that formats incorporating multiple antigen-binding domains may include VH and/or VL sequences in any combination or orientation. For example, the antigen-binding fragment can comprise the format VL-linker VH-linker VL-linker VH, VH-linker VL-linker VL-linker VH, or VL-linker VH-linker VH-linker VL.

In embodiments including two VH domains and/or two VL domains, one or more VH domain or VL domains or one or more or two or more CDRs therein are according to the sequences set forth in SEQ ID NOs.: 1-574 and 701-903 of Table 1 and the Sequence Listing and, optionally, according to the combinations set forth for specific antibodies in Table 2 and Table 20. In such embodiments, one or more VH or VL or one or more or two or more CDRs may also be according to the sequences set forth in SEQ ID NOs.: 575-655 of Table 1 and the Sequence Listing and, optionally, for anti-RSV antibodies, according to the combinations set forth for specific antibodies in Table 3, or, for anti-RSV/MPV antibodies, according to the combinations set forth for specific antibodies in Table 4.

More specifically, the one or more VH may bind RSV-F and may comprise CDRs of in a VH sequence according to any one of SEQ ID NOs.: 576, 586, 591, 600, 604, 613, and 617 and the one or more VL may bind RSV-F and may comprise a VL sequence according to any one of SEQ ID NOs.: 581, 588, 596, 602, 609, 615, and 622 as determined using any known CDR numbering method, including the Kabat, Chothia, EU, Martin (Enhanced Chothia), Contact, AbM, CCG, EU, or AHo, numbering method or a combination of two or more of theseIMGT, Martin (Enhanced Chothia), Contact, North, and AHo numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software.

In certain embodiments, the CDRH1, CDRH2, CDRH3, CDRL1, CDRL2, and CDRL3 may comprise or consist of SEQ ID NOs.: 577, 592, 605, or 618; SEQ ID NOs.: 578, 593, 606, or 619; SEQ ID NOs.: 579, 594, 607, or 620; SEQ ID NOs.: 582, 597, 610, or 623; SEQ ID NOs.: 583, 143, 268, or 624, or SEQ ID NOs.: 584, 598, 611, or 625, respectively or, in each case, a functional variant thereof comprising one, two, or three amino acid substitutions, one or more of which substitutions is optionally a conservative substitution and/or is a substitution to a germline-encoded amino acid, or an amino acid sequence having at least 80% (e.g., 80%, 85%, 90%, 91%, 92%, 93%, 94%, 95%, 96%, 97%, 98%, 99%, or more) identity to the amino acid sequence.

Monospecific or multispecific antibodies or antigen-binding fragments of the present disclosure constructed comprise any combination of the VH and VL sequences and/or any combination of the CDRH1, CDRH2, CDRH3, CDRL1, CDRL2, and CDRL3 sequences disclosed herein in SEQ ID NOs.: 1-574 and 701-903 of Table 1 and the Sequence Listing and, optionally, according to the combinations set forth for specific antibodies in Table 2 and Table 20. A bispecific or multispecific antibody or antigen-binding fragment may, in some embodiments, comprise one, two, or more antigen-binding domains (e.g., a VH and a VL) of the instant disclosure, one or more VH or VL or one or more or two or more CDRs of which may also be according to the sequences set forth in SEQ ID NOs.: 575-655 of Table 1 and the Sequence Listing and, optionally, for anti-RSV antibodies, according to the combinations set forth for specific antibodies in Table 3 and the Sequence Listing, or, for anti-RSV/MPV antibodies, according to the combinations set forth for specific antibodies in Table 4 and the Sequence Listing. Two or more binding domains may be present that bind to the same or a different RSV-F and/or MPV-F epitope, and a bispecific or multispecific antibody or antigen-binding fragment as provided herein can, in some embodiments, comprise a further RSV-F- and/or MPV-F-specific binding domain, and/or can comprise a binding domain that binds to a different antigen or pathogen altogether.

In some embodiments, the antibody or antigen-binding fragment comprises a heavy chain, and can be, for example, IgG1m3 comprising M428L and N434S mutations in the heavy chain. In some embodiments, the antibody or antigen-binding fragment comprises a light chain.

A light chain can be, for example, a kappa light chain or a lambda light chain.

In any of the presently disclosed embodiments, the antibody or antigen-binding fragment can be multispecific; e.g., bispecific, trispecific, or the like.

In certain embodiments, the antibody or antigen-binding fragment is at least bispecific and comprises at least a VH and VL, or the three CDRs of the VH and the three CDRs of the VL of at least one of i) MPK65-v2-v1.2, MPK65-v2-v3.1, MPK176-v1.3, MPK76-v43., MPK201-v1.2, and MPK 201-v1.4; or ii) the VH and a VL amino acid sequences set forth in any one of SEQ ID NOs.: 136 and 851; 814 and 141; 233 and 858; 837 and 858; 357 and 847; or 899 and 360, respectively.

In certain embodiments, the antibody or antigen-binding fragment is at least bispecific and comprises at least a VH and VL, or the three CDRs of the VH and the three CDRs of the VL of MPK190-v1.3 or the VH and VL amino acid sequences as set forth in SEQ ID NOs.: 702 and 704, respectively.

In certain embodiments, the antibody or antigen-binding fragment is at least bispecific and comprises at least (A) a first VH and VL, or first set of six CDRs, wherein first set of six CDRs are ther three HCDRs and the three LCDRs of the VH and VL of at least one of i) MPK65-v2-v1.2, MPK65-v2-v3.1, MPK176-v1.3, MPK76-v43., MPK201-v1.2, and MPK 201-v1.4; or ii) a VH and a VL set forth in any one of SEQ ID NOs.: 136 and 851; 814 and 141; 233 and 858; 837 and 858; 357 and 847; and 899 and 360, respectively; and (B) at least a second VH and VL, or a second set of six CDRs, wwherein the second set of six CDRs are the three HCDRs and the three LCDRs of the VH and VL of MPK190-v1.3, or a VH and VL as set forth in SEQ ID NOs.: 702 and 704.

In some embodiments, the antibody or antigen-binding fragment may further comprise a Fc polypeptide or fragment thereof that comprises or consists of amino acid sequences having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to, or comprises or consists of, the amino acid sequences set forth in and one of SEQ ID NOs.: 664-700, more particularly any one of SEQ ID NOs.:670-700.

It will be understood that, for example, production in a mammalian cell line can remove one or more C-terminal lysine of an antibody heavy chain (see, e.g., Liu et al. mAbs 6(5):1145-1154 (2014)). Produciton can also remove one or more C-termal glycine of an antibody heavy chaing. Accordingly, an antibody or antigen-binding fragment of the present disclosure can comprise a heavy chain, a CH1-CH3, a CH3, or an Fc polypeptide wherein a C-terminal lysine and/or glycine residue is present or is absent; in other words, encompassed are embodiments where the C-terminal residue of a heavy chain, a CH1-CH3, or an Fc polypeptide is not a lysine or a glycine, and embodiments where a lysine or a glycine is the C-terminal residue. In certain embodiments, a composition comprises a plurality of an antibody and/or an antigen-binding fragment of the present disclosure, wherein one or more antibody or antigen-binding fragment does not comprise a lysine and/or glycine residue at the C-terminal end of the heavy chain, CH1-CH3, or Fc polypeptide, and wherein one or more antibody or antigen-binding fragment comprises a lysine or glycine residue at the C-terminal end of the heavy chain, CH1-CH3, or Fc polypeptide.

In certain embodiments, the antibody or antigen-binding fragment comprises a Fc polypeptide, or a fragment thereof. The “Fc” fragment or Fc polypeptide comprises the carboxy-terminal portions (i.e., the CH2 and CH3 domains of IgG) of both antibody H chains held together by disulfides. An Fc may comprise a dimer comprised of two Fc polypeptides (i.e., two CH2-CH3 polypeptides). Antibody “effector functions” refer to those biological activities attributable to the Fc region (a native sequence Fc region or amino acid sequence variant Fc region) of an antibody, and vary with the antibody isotype. Examples of antibody effector functions include: Cq binding and complement dependent cytotoxicity; Fc receptor binding; antibody-dependent cell-mediated cytotoxicity (ADCC); phagocytosis; down regulation of cell surface receptors (e.g., B cell receptor); and B cell activation. As discussed herein, modifications (e.g., amino acid substitutions) may be made to an Fc domain in order to modify (e.g., improve, reduce, or ablate) one or more functionality of an Fc-containing polypeptide (e.g., an antibody of the present disclosure). Such functions include, for example, Fc receptor (FcR) binding, antibody half-life modulation (e.g., by binding to FcRn), ADCC function, protein A binding, protein G binding, and complement binding. Amino acid modifications that modify (e.g., improve, reduce, or ablate) Fc functionalities include, for example, the T250Q/M428L, M252Y/S254T/T256E, H433K/N434F, M428L/N434S, M428L/N434A, E233P/L234V/L235A/G236+A327G/A330S/P33IS, E333A, S239D/A330L/1332E, P257I/Q311, K326W/E333S, S239D/1332E/G236A, N297Q, K322A, S228P, L235E+E318A/K320A/K322A, L234A/L235A (also referred to herein as “LALA”), and L234A/L235A/P329G mutations, which mutations are summarized and annotated in “Engineered Fc Regions”, published by InvivoGen (2011), and are incorporated herein by reference.

For example, to activate the complement cascade, the C1q protein complex can bind to two or more molecules of IgGI or one molecule of IgM when the immunoglobulin molecule(s) is attached to the antigenic target (Ward, E. S., and Ghetie, V., Ther. Immunol. 2 (1995) 77-94).

Burton, D. R., described (Mol. Immunol. 22 (1985) 161-206) that the heavy chain region comprising amino acid residues 318 to 337 is involved in complement fixation. Duncan, A. R., and Winter, G. (Nature 332 (1988) 738-740), using site directed mutagenesis, reported that Glu318, Lys320 and Lys322 form the binding site to C1q. The role of Glu318, Lys320 and Lys 322 residues in the binding of C1q was confirmed by the ability of a short synthetic peptide containing these residues to inhibit complement mediated lysis.

For example, FcR binding can be mediated by the interaction of the Fc moiety (of an antibody) with Fc receptors (FcRs), which are specialized cell surface receptors on cells including hematopoietic cells. Fc receptors belong to the immunoglobulin superfamily, and shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC; Van de Winkel, J. G., and Anderson, C. L., J. Leukoc. Biol. 49 (1991) 511-524). FcRs are defined by their specificity for immunoglobulin classes; Fc receptors for IgG antibodies are referred to as FcγR, for IgE as FcR, for IgA as FcaR and so on and neonatal Fc receptors are referred to as FcRn. Fc receptor binding is described for example in Ravetch, J. V., and Kinet, J.

P., Annu. Rev. Immunol. 9 (1991) 457-492; Capel, P. J., et al., Immunomethods 4 (1994) 25-34; de Haas, M., et al., J Lab. Clin. Med. 126 (1995) 330-341; and Gessner, J. E., et al., Ann.

### Hematol. 76 (1998) 231-248.

Cross-linking of receptors by the Fc domain of native IgG antibodies (FcγR) triggers a wide variety of effector functions including phagocytosis, antibody-dependent cellular cytotoxicity, and release of inflammatory mediators, as well as immune complex clearance and regulation of antibody production. Fc moieties providing cross-linking of receptors (e.g., FcγR) are contemplated herein. In humans, three classes of FcγR have been characterized to-date, which are: (i) FcγRI (CD64), which binds monomeric IgG with high affinity and is expressed on macrophages, monocytes, neutrophils and eosinophils; (ii) FcγRII (CD32), which binds complexed IgG with medium to low affinity, is widely expressed, in particular on leukocytes, is believed to be a central player in antibody-mediated immunity, and which can be divided into FcγRIIA, FcγRIIB and FcγRIIC, which perform different functions in the immune system, but bind with similar low affinity to the IgG-Fc, and the ectodomains of these receptors are highly homologous; and (iii) FcγRIII (CD16), which binds IgG with medium to low affinity and has been found in two forms: FcγRIIIA, which has been found on NK cells, macrophages, eosinophils, and some monocytes and T cells, and is believed to mediate ADCC; and FcγRIIIB, which is highly expressed on neutrophils.

FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and eosinophils.

Importantly, it has been shown that 75% of all FcγRIIB is found in the liver (Ganesan, L. P. et al., 2012: “FcγRIIb on liver sinusoidal endothelium clears small immune complexes,” Journal of Immunology 189: 4981-4988). FcγRIIB is abundantly expressed on Liver Sinusoidal Endothelium, called LSEC, and in Kupffer cells in the liver and LSEC are the major site of small immune complexes clearance (Ganesan, L. P. et al., 2012: FcγRIIb on liver sinusoidal endothelium clears small immune complexes. Journal of Immunology 189: 4981-4988).

In some embodiments, the antibodies disclosed herein and the antigen-binding fragments thereof comprise an Fc polypeptide or fragment thereof for binding to FcγRIIb, in particular an Fc region, such as, for example IgG-type antibodies. Moreover, it is possible to engineer the Fc moiety to enhance FcγRIIB binding by introducing the mutations S267E and L328F as described by Chu, S. Y. et al., 2008: Inhibition of B cell receptor-mediated activation of primary human B cells by coengagement of CD19 and FegammaRIIb with Fe-engineered antibodies. Molecular Immunology 45, 3926-3933. Thereby, the clearance of immune complexes can be enhanced (Chu, S., et al., 2014: Accelerated Clearance of IgE In Chimpanzees Is Mediated By Xmab7195, An Fc-Engineered Antibody With Enhanced Affinity For Inhibitory Receptor FcγRIIb. Am J Respir Crit, American Thoracic Society International Conference Abstracts). In some embodiments, the antibodies of the present disclosure, or the antigen-binding fragments thereof, comprise an engineered Fc moiety with the mutations S267E and L328F, in particular as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933.

On B cells, FcγRIIB may function to suppress further immunoglobulin production and isotype switching to, for example, the IgE class. On macrophages, FcγRIIB is thought to inhibit phagocytosis as mediated through FcγRIIA. On eosinophils and mast cells, the B form may help to suppress activation of these cells through IgE binding to its separate receptor.

Regarding FcγRI binding, modification in native IgG of one or more of E233-G236, P238, D265, N297, A327 and P329 reduces binding to FcγRI. IgG2 residues at positions 233-236, substituted into corresponding positions IgGI and IgG4, reduces binding of IgGI and IgG4 to FcγRI by 103-fold and eliminated the human monocyte response to antibody-sensitized red blood cells (Armour, K. L., et al. Eur. J. Immunol. 29 (1999) 2613-2624).

Regarding FcγRII binding, reduced binding for FcγRIIA is found, e.g., for IgG mutation of one or more of E233-G236, P238, D265, N297, A327, P329, D270, Q295, A327, R292 and K414.

Two allelic forms of human FcγRIIA are the “H131” variant, which binds to IgGI Fc with higher affinity, and the “R131” variant, which binds to IgGI Fc with low affinity. See, e.g., Bruhns et al., Blood 113:3716-3725 (2009).

Regarding FcγRIII binding, reduced binding to FcγRIIIA is found, e.g., for mutation of one or more of E233-G236, P238, D265, N297, A327, P329, D270, Q295, A327, S239, E269, E293, Y296, V303, A327, K338 and D376. Mapping of the binding sites on human IgGI for Fc receptors, the above-mentioned mutation sites, and methods for measuring binding to FcγRI and FcγRIIA, are described in Shields, R. L., et al., J. Biol. Chem. 276 (2001) 6591-6604.

Two allelic forms of human FcγRIIIA are the “F158” variant, which binds to IgGI Fc with lower affinity, and the “V158” variant, which binds to IgGI Fc with higher affinity. See, e.g., Bruhns et al., Blood 113:3716-3725 (2009).

Regarding binding to FcγRII, two regions of native IgG Fc appear to be involved in interactions between FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g. in a region of P331 (Wines, B. D., et al., J. Immunol. 2000; 164: 5313-5318). Moreover, FcγRI appears to bind to the same site on IgG Fc, whereas FcRn and Protein A bind to a different site on IgG Fc, which appears to be at the CH2-CH3 interface (Wines, B. D., et al., J. Immunol. 2000; 164: 5313-5318).

Also contemplated are mutations that increase binding affinity of an Fc polypeptide or fragment thereof of the present disclosure to a (i.e., one or more) Fcγ receptor (e.g., as compared to a reference Fc polypeptide or fragment thereof or containing the same that does not comprise the mutation(s)). See, e.g., Delillo and Ravetch, Cell 161(5):1035-1045 (2015) and Ahmed et al., J. Struc. Biol. 194(1):78 (2016), the Fc mutations and techniques of which are incorporated herein by reference.

In any of the herein disclosed embodiments, an antibody or antigen-binding fragment can comprise a Fc polypeptide or fragment thereof comprising a mutation selected from G236A; S239D; A330L; and 1332E; or a combination comprising any two or more of the same; e.g., S239D/I332E; S239D/A330L/I332E; G236A/S239D/I332E; G236A/A330L/I332E (also referred to herein as “GAALIE”); or G236A/S239D/A330L/I332E. In some embodiments, the Fc polypeptide or fragment thereof does not comprise S239D. In some embodiments, the Fc polypeptide or fragment thereof comprises S at position 239 (EU numbering). In some embodiments, the Fc polypeptide or fragment thereof comprises the amino acid sequences set forth in SEQ ID NOs.: 672-678.

In certain embodiments, the Fc polypeptide or fragment thereof may comprise or consist of at least a portion of an Fc polypeptide or fragment thereof that is involved in FcRn binding. In certain embodiments, the Fc polypeptide or fragment thereof comprises one or more amino acid modifications that improve binding affinity for (e.g., enhance binding to) FcRn (e.g., at a pH of about 6.0) and, in some embodiments, thereby extend in vivo half-life of a molecule comprising the Fc polypeptide or fragment thereof (e.g., as compared to a reference Fc polypeptide or fragment thereof or antibody that is otherwise the same but does not comprise the modification(s)). In certain embodiments, the Fc polypeptide or fragment thereof comprises or is derived from a IgG Fc and a half-life-extending mutation comprises any one or more of: M428L; N434S; N434H; N434A; N434S; M252Y; S254T; T256E; T250Q; P257I Q311I; D376V; T307A; E380A (EU numbering). In certain embodiments, a half-life-extending mutation comprises M428L/N434S (also referred to herein as “MLNS”, “LS”, “_LS”, and “-LS”). In certain embodiments, the half-life extending mutation is in a Fc polypeptide or fragment thereof comprising or consisting of the amino acid sequences set forth in SEQ ID NOs.: 679-684. In certain embodiments, a half-life-extending mutation comprises M252Y/S254T/T256E. In certain embodiments, a half-life-extending mutation comprises T250Q/M428L. In certain embodiments, a half-life-extending mutation comprises P257I/Q311I. In certain embodiments, a half-life-extending mutation comprises P257I/N434H. In certain embodiments, a half-life-extending mutation comprises D376V/N434H. In certain embodiments, a half-life-extending mutation comprises T307A/E380A/N434A. In certain embodiments, a half-life-extending mutation comprises M428L/N434A (also referred to herein as “MLNA”, “LA”, _LA”, and “-LA”). In certain embodiments, the half-life extending mutation is in a Fc polypeptide or fragment thereof comprising or consisting of the amino acid sequences set forth in SEQ ID NOs.: 685-690.

In some embodiments, an antibody or antigen-binding fragment includes a Fc moiety that comprises the substitution mutations M428L/N434S or M428L/N434A. In some embodiments, an antibody or antigen-binding fragment includes a Fc polypeptide or fragment thereof that comprises the substitution mutations G236A/A330L/I332E. In certain embodiments, an antibody or antigen-binding fragment includes a (e.g., IgG) Fc moiety that comprises a G236A mutation, an A330L mutation, and a 1332E mutation (GAALIE), and does not comprise a S239D mutation (e.g., comprises a native S at position 239). In particular embodiments, an antibody or antigen-binding fragment includes an Fc polypeptide or fragment thereof that comprises the substitution mutation: M428L/N434S and G236A/A330L/I332E, (and may comprises or consist of the amino acid sequences set forth in SEQ ID NOs.: 691-695) and, optionally does not comprise S239D (e.g., comprises S at 239). In particular embodiments, an antibody or antigen-binding fragment includes an Fc polypeptide or fragment thereof that comprises the substitution mutation: M428L/N434A and G236A/A330L/I332E, (and may comprises or consist of the amino acid sequences set forth in SEQ ID NOs.: 696-700) and, optionally does not comprise S239D (e.g., comprises S at 239). In certain embodiments, an antibody or antigen-binding fragment includes a Fc polypeptide or fragment thereof that comprises the substitution mutations: M428L/N434S (or M428L/N434A) and G236A/S239D/A330L/I332E.

In some embodiments, an antibody or antigen-binding fragment (described further herein) is provided that comprises, in a(n e.g. human) IgGI heavy chain, the amino acid mutation(s) set forth in any one of (i)-(xviii): (i) G236A, L328V, and Q295E; (ii) G236A, P230A, and Q295E; (iii) G236A, R292P, and 1377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E, wherein the numbering of amino acid residues is according to the EU index as set forth in Kabat. In certain embodiments, the antibody or antigen-binding fragment is afucosylated. In some embodiments, the antibody or antigen-binding fragment further comprises one or more mutation that enhances binding to a human FcRn, such as M428L and N434S mutations or M428L and N434A mutations (EU numbering) or any other mutation(s) that enhance binding to a human FcRn, such as those described herein. In certain embodiments, the antibody or antigen-binding fragment is afucosylated.

In specific embodiments, the antibody or antigen-binding fragment comprises i) a VH and a VL as set forth anywhere herein, respectively; or (iii) a CDRH1, CDRH2, CDRH3, CDRL1, CDRL2, and CDRL3 as set forth anywhere herein or as determined by any CDR determination scheme disclosed herein, and (A) a Fc moiety that comprises the substitution mutations: (i) G236A, L328V, and Q295E; (ii) G236A, P230A, and Q295E; (iii) G236A, R292P, and 1377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E; (xix) M428L/N434S; (xx) M428L/N434A; (xxi) G236A/A330L/I332E/M428L/N434S; (xxii) G236A/A330L/I332E/M428L/N434A; or (xxiii) any two or more of (i)-(xxii); or (B) an Fc moiety that comprises or consists of a Fc polypeptide or fragment thereof that comprises or consists of amino acid sequences having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs.: 664-700, optionally other than naturally occurring variants thereof, or that comprises or consists of, the amino acid sequences set forth in any one of SEQ ID NOs.: 672-700.

In certain embodiments, the antibody or antigen-binding fragment comprises a mutation that alters glycosylation, wherein the mutation that alters glycosylation comprises N297A, N297Q, or N297G, and/or the antibody or antigen-binding fragment is partially or fully aglycosylated and/or is partially or fully afucosylated. Host cell lines and methods of making partially or fully aglycosylated or partially or fully afucosylated antibodies and antigen-binding fragments are known (see, e.g., PCT Publication No. WO 2016/181357; Suzuki et al. Clin.

Cancer Res. 13(6):1875-82 (2007); Huang et al. MAbs 6:1-12 (2018)).

An antibody or antigen-binding fragment of the present disclosure can be fucosylated (e.g., comprising one or more fucosyl moiety, and typically comprising a native (wild-type) fucosylation pattern or a fucosylation pattern that includes one or more additional, or fewer, fucosyl moieties as compared to native), or can be afucosylated. In particular, native IgGI antibodies carry a glycan site at N297, and this is typically the only site where a core fucose moiety may be found in the antibody, though some glycan sites may arise through mutation (e.g. in the variable domains) during antibody development. Fucosylation of an Fc polypeptide or fragment thereof, or of an antibody, can be effected by introducing amino acid mutations to introduce or disrupt a fucosylation site (e.g. a mutation at N297, such as N297Q or N297A, to disrupt formation of a glycan that can include a core fucose moiety), though typically it is preferred to maintain N297 and the glycan thereof, such as by expressing the polypeptide in a host cell which has been genetically engineered to lack the ability (or have an inhibited or compromised ability) to fucosylate the polypeptide; by expressing the polypeptide under conditions in which a host cell is impaired in its ability to fucosylate the polypeptide (e.g., in the presence of 2-fluoro-L-fucose (2FF)), or the like. An afucosylated polypeptide can comprise no fucose moieties, or substantially no fucose moieties, and/or can be expressed by a host cell that is genetically engineered to lack the ability (or have an inhibited or compromised ability) to fucosylate the polypeptide and/or can be expressed under conditions in which a host cell is impaired in its ability to fucosylate the polypeptide (e.g., in the presence of 2-fluoro-L-fucose (2FF)). In some embodiments, a polypeptide does not comprise a core fucose moiety at Asn297. In some embodiments, afucosylated polypeptides have increased binding to FcγRIIIA. In some contexts, addition of 2FF to a culture media comprising host cells expressing an antibody results in about 85% or more of the antibodies not carrying a fucose moiety.

Accordingly, a plurality of antibodies may be described as “afucosylated” when the plurality was produced in the presence of 2FF or like reagent. In some contexts, a plurality of polypeptides or antibodies may be described as, for example, afucosylated, meaning that about 85% or more of the single polypeptide or antibody molecules of the plurality do not comprise a fucose moiety. In certain preferred embodiments, an afucosylated antibody or polypeptide or a population or a plurality thereof comprises an asparagine (N) at EU position 297. Fucosylation or lack thereof can be assessed using, for example, mass spectrometry (e.g. Electrospray mass spectrometry (ESI-MS)). In some embodiments, compositions are provided that comprise a plurality of any one or more of the presently disclosed polypeptides, wherein the composition comprises afucosylated polypeptides.

In certain embodiments, the antibody or antigen-binding fragment is elicits continued protection in vivo in a subject even once no detectable levels of the antibody or antigen-binding fragment can be found in the subject (i.e., when the antibody or antigen-binding fragment has been cleared from the subject following administration). Such protection is referred to herein as a vaccinal effect. Without wishing to be bound by theory, it is believed that dendritic cells can internalize complexes ofantibody and antigen and thereafter induce or contribute to an endogenous immune response against antigen. In certain embodiments, an antibody or antigen-binding fragment comprises one or more modifications, such as, for example, mutations in the Fc comprising G236A, A330L, and 1332E, that activate dendritic cells that may induce, e.g., T cell immunity to the antigen.

In certain embodiments, an antibody or antigen-binding fragment fo the present disclosure comprises an Fc variant selected from the Fc variants summarized in Table A (see also PCT Publication No. WO 2022/251119). In certain embodiments, the Fc variant, or the antibody or antigen-binding fragment, is fucosylated. In other embodiments, the Fc variant, or the antibody or antigen-binding fragment, is afucosylated.

TABLE A
Certain Fc Variants (fucosylated unless otherwise indicated) and Properties Thereof

| Variant | |
|---|---|
| (substitution mutation(s) | Certain properties of the indicated variant(s), as compared to |
| vs. wild-type human IgG1 Fc) | fucosylated wild-type human IgG1 |
| G236A_L328V_Q295E | Increased binding to human FcγRIIa (H131 allele and R131 allele); |
| G236A_P230A_Q295E | comparable or decreased binding to human FcγRIIb (e.g. by MSD |
| G236A_R292P_I377N | assay and/or surface plasmon resonance); increased ratio of: |
| G236A_K334A_Q295E | binding to human FcγRIIa (H131 allele or R131 allele) versus |
| G236S_R292P_Y300L | binding to human FcγRIIb; comparable binding to human FcRn; |
| | comparable production titer; increased signaling in a host cell via |
| | FcγRIIa and/or decreased signaling in a host cell via FcγRIIb; Tm |
| | within 12° C. or less of wild-type; G236S_R292P_Y300L has |
| | improved binding to C1q |
| G236A_Y300L | Increased binding to human FcγRIIa (H131 (over 18-fold) and |
| | R131 (over 4-fold)); similar binding to human FcγRIIb or reduced |
| | binding to human FcγRIIb (e.g. as measured by surface plasmon |
| | resonance); increased ratio of: binding to human FcγRIIa (H131 or |
| | R131) versus binding to human FcγRIIb; comparable binding to |
| | human FcRn; comparable production titer; increased signaling in a |
| | host cell via FcγRIIa and/or decreased signaling in a host cell via |
| | FcγRIIb; Tm within 4.5° C. of wild-type |
| G236A_R292P_Y300L | Increased binding to human FcγRIIa (H131 (over 14-fold) and |
| | R131 (over 2.7-fold)); similar binding to human FcγRIIb; increased |
| | ratio of: binding to human FcγRIIa (H131 or R131) versus binding |
| | to human FcγRIIb; increased binding to human FcγRIIIa (V158 |
| | allele and F158 allele); comparable binding to human FcRn; |
| | comparable production titer; increased signaling in a host cell via |
| | FcγRIIa and/or FcγRIIIa, and/or decreased signaling in a host cell |
| | via FcγRIIb; increased signaling in a host cell via FcγRIIa and/or |
| | decreased signaling in a host cell via FcγRIIb; Tm within 4° C. of |
| | wild-type; comparable binding to human C1q |
| G236S_G420V_G446E_L309T | Increased binding to human FcγRIIa; decreased binding to human |
| G236A_R292P | FcγRIIb (less than 0.5-fold); increased ratio of: binding to human |
| | FcγRIIa (H131 or R131) versus binding to human FcγRIIb; |
| | comparable binding to human FcRn; comparable production titer; |
| | increased signaling in a host cell via FcγRIIa and/or FcγRIIIa, |
| | and/or decreased signaling in a host cell via FcγRIIb; Tm within |
| | 4° C. or less of wild-type |
| R292P_Y300L | Increased binding to human FcγRIIIa (V158 and F158); increased |
| | binding to human C1q; Tm within 4° C. of wild-type |
| Y300L | Increased binding to human C1q |
| E345K_G236S_L235Y_S267E | |
| E272R_L309T_S219Y_S267E | |
| G236Y | |
| G236W | |
| F243L_G446E_P396L_S267E | |
| G236A (afucosylated) | Increased binding to human FcγRIIa (H131) and mouse FcγRIIa |
| | (R131), decreased binding human FcγRIIb, increased binding to |
| | human FcγRIIIa (V158) and mouse FcγRIIIa (F158), increased |
| | binding to human FcγRIIIb, somewhat decreased binding to human |
| | FcRn, Tm within 0.15° C. of wild-type or within 0.9° C. of wild-type |
| | or within 0.8° C. of wild-type or within 0.7° C. of wild-type |
| S239D_H268E_G236A | Increased binding to and signaling via all human FcγRs tested: |
| | FcγRIIA (H131); FcγRIIA (R131); FcγRIIB; FcγRIIIA (V158); |
| | FcγRIIIA (F158); FcγRIIIB; additionally, when anti-HBV antibody |
| | bearing S239D_H268E_G236A_M428L_N434S was combined |
| | with hBsAg, the immune complexes formed thereby were |
| | incubated with MoDCs; subsequent incubation of the MoDCs with |
| | donor CD4+ T cells resulted in an increased percentage of |
| | NFAT+CD69+ CD3+CD4+ T cells as compared to antibodies |
| | bearing M428L_N434S only. |

In some embodiments, an anti-parvovirus antibody or antigen-binding fragment is provided that comprises, in a(n e.g. human) IgGI heavy chain, the amino acid mutation(s) set forth in any one of (i)-(xviii): (i) G236A, L328V, and Q295E; (ii) G236A, P230A, and Q295E; (iii) G236A, R292P, and 1377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E, wherein the numbering of amino acid residues is according to the EU index as set forth in Kabat. In certain embodiments, the antibody or antigen-binding fragment is afucosylated. In some embodiments, the antibody or antigen-binding fragment further comprises one or more mutation that enhances binding to a human FcRn, such as M428L and N434S mutations or M428L and N434A mutations (EU numbering) or any other mutation(s) that enhance binding to a human FcRn, such as those described herein. In certain embodiments, the antibody or antigen-binding fragment is afucosylated.

In any of the presently disclosed embodiments, the antibody or antigen-binding fragment comprises a Fc polypeptide or a fragment thereof, including a CH2 (or a fragment thereof, a CH3 (or a fragment thereof), or a CH2 and a CH3, wherein the CH2, the CH3, or both can be of any isotype and may contain amino acid substitutions or other modifications as compared to a corresponding wild-type CH2 or CH3, respectively. In certain embodiments, a Fc of the present disclosure comprises two CH2-CH3 polypeptides that associate to form a dimer.

In any of the presently disclosed embodiments, the antibody or antigen-binding fragment can be monoclonal. The term “monoclonal antibody” (mAb) as used herein refers to an antibody obtained from a population of substantially homogeneous antibodies, i.e., individual antibodies comprising the population are identical except for possible naturally occurring mutations that may be present, in some cases in minor amounts. Monoclonal antibodies are highly specific, being directed against a single antigenic site. Furthermore, in contrast to polyclonal antibody preparations that include different antibodies directed against different epitopes, each monoclonal antibody is directed against a single epitope of the antigen.

In addition to their specificity, the monoclonal antibodies are advantageous in that they may be synthesized uncontaminated by other antibodies. The term “monoclonal” is not to be construed as requiring production of the antibody by any particular method. For example, monoclonal antibodies useful in the present invention may be prepared by the hybridoma methodology first described by Kohler et al., Nature 256:495 (1975), or may be made using recombinant DNA methods in bacterial, eukaryotic animal, or plant cells (see, e.g., U.S. Pat. No. 4,816,567).

Monoclonal antibodies may also be isolated from phage antibody libraries using the techniques described in Clackson et al., Nature, 352:624-628 (1991) and Marks et al., J Mol. Biol., 222:581-597 (1991), for example. Monoclonal antibodies may also be obtained using methods disclosed in PCT Publication No. WO 2004/076677A2.

Antibodies and antigen-binding fragments of the present disclosure include “chimeric antibodies” in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, U.S. Pat. Nos. 4,816,567; 5,530,101 and 7,498,415; and Morrison et al., Proc. Natl. Acad. Sci. USA, 81:6851-6855 (1984)). For example, chimeric antibodies may comprise human and non-human residues. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr Op. Struct. Biol. 2:593-596 (1992). Chimeric antibodies also include primatized and humanized antibodies.

A “humanized antibody” is generally considered to be a human antibody that has one or more amino acid residues introduced into it from a source that is non-human. These non-human amino acid residues are typically taken from a variable domain. Humanization may be performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting non-human variable sequences for the corresponding sequences of a human antibody. Accordingly, such “humanized” antibodies are chimeric antibodies (U.S. Pat. Nos. 4,816,567; 5,530,101 and 7,498,415) wherein substantially less than an intact human variable domain has been substituted by the corresponding sequence from a non-human species.

In some instances, a “humanized” antibody is one which is produced by a non-human cell or animal and comprises human sequences, e.g., Hc domains.

A “human antibody” is an antibody containing only sequences that are present in an antibody that is produced by a human (i.e., sequences that are encoded by human antibody-encoding genes). However, as used herein, human antibodies may comprise residues or modifications not found in a naturally occurring human antibody (e.g., an antibody that is isolated from a human), including those modifications and variant sequences described herein.

These are typically made to further refine or enhance antibody performance. In some instances, human antibodies are produced by transgenic animals. For example, see U.S. Pat. Nos. 5,770,429; 6,596,541 and 7,049,426.

In certain embodiments, an antibody or antigen-binding fragment of the present disclosure is chimeric, humanized, or human.

In some embodiments, various pharmacokinetic (“PK”) parameters are used to describe or characterize the antibodies or antigen-binding fragments provided herein. Details regarding collection of antibody serum concentrations for purpose of evaluating PK parameters are described in association with the Examples herein. The term “t₁₁₂” or “half-life” refers to the elimination half-life of the antibody included in the pharmaceutical composition administered to a subject. The term “Ciₐₛₜ” generally refers to the last measurable plasma concentration (i.e., subsequent thereto, the substance is not present at a measurable concentration in plasma).

MPK190-v1.3 Antibodies In some embodiments, an antibody or antigen-binding fragment of the present disclosure is a MPK190-v1.3 antibody or antigen-binding fragment therof. Such an antibody or antigen-binding fragment thereof may bind and/or neutralize both RSV and MPV.

In some embodiments, a MPK190-v1.3 antibody may have a VH, a VL, a HC, a LC, CDRH1-H3, and/or CDRL1-L3 comprising or consisting of amino acid sequences or encoded by polynucleotides having sequences as follows:

| | SEQ ID NOs. | | | | | |
|---|---|---|---|---|---|---|
| | CDRH1-3 | VH (aa / | HC (aa / | CDRL1-3 | VL (aa / | |
| Ab | (aa) | nt) | nt) | (aa) | nt) | LC (aa / nt) |
| MPK190-v1.3 | 121, 122, | 702 / 701 | 723 / 722 | 18, 705, 127 | 704 / 703 | 725 / 724 (nt) / |
| | 123 | | | | | 904 (nt) |

In some embodiments, the VH and VL for a MPK190-v1.3 antibody or antigen-binding fragment that binds RSV-F and MPV-F and/or neutralizes RSV and/or MPV comprise or consist of a VH and VL having the sequences of SEQ ID NOs.: 702 and 704, respectively.

In certain embodiments, an antibody or antigen-binding fragment of the present disclosure binds RSV-F and MPV-F and/or neutralizes RSV and MPV and:
(i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in SEQ ID NO.: 702, wherein sequence variation is optionally limited to one or more framework regions and/or sequence variation comprises one or more substitution to a germline-encoded amino acid; and/or
(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in SEQ ID NO.: 704, wherein sequence variation is optionally limited to one or more framework regions and/or sequence variation comprises one or more substitution to a germline-encoded amino acid.

In some embodiments, variation as compared to SEQ ID NO.: 702 or SEQ ID NO.: 704 is limited to one or more framework region. In some embodiments, the variation comprises or consists of one, two, three, four, five, six, seven, eight, nine, or ten amino acid substitutions (any or all of which may be a conservative substitution), insertions, or deletions in the VH, in the VL, or in both. In some embodiments, the variation comprises one or more amino acid substitution, insertion, and/or deletion of an amino acid that is two, three, four, five, six, seven, eight, nine, ten, eleven, twelve, thirteen, fourteen, fifteen, or more amino acid positions away from an N-terminal and/or a C-terminal amino acid of a CDR.

Framework regions can be identified according to a numbering scheme (e.g., IMGT, Kabat, Chothia, North, EU, Martin (Enhanced Chothia), Contact, AbM, CCG, EU, or AHo, or a combination of two or more of these). The CDRs may be identified within a variable domain or within a heavy or light chain according to a numbering scheme or a combination of numbering schemes, and, preferably, the FRs may be identified using the same numbering scheme or combination of numbering schemes.

For example, in some embodiments, an antibody or antigen-binding fragment comprises CDRH1, CDRH2, CDRH3, CDRL1, CDRL2, and CDRL3 of MPK190-v1.3, wherein the CDRH1, CDRH2, CDRH3, CDRL1, CDRL2, and CDRL3 are according to IMGT (optionally IMGT-junction for CDRH3 and CDRL3), and the antibody or antigen-binding fragment further comprises one or more framework sequence from MPK190-v1.3 and/or one or more framework sequence that is a variant of a MPK190-v1.3framework sequence, wherein the one or more framework sequence of MPK190-v1.3 is according to IMGT (and if IMGT-junction is used for CDRH3 and CDRL3, accounting for this).

In other embodiments, an antibody or antigen-binding fragment comprises CDRH1, CDRH2, CDRH3, CDRL1, CDRL2, and CDRL3 of MPK190-v1.3, wherein the CDRH1, CDRH2, CDRH3, CDRL1, CDRL2, and CDRL3 are according to Kabat, and the antibody or antigen-binding fragment further comprises one or more framework sequence from [MPK190-v1.3 and/or one or more framework sequence that is a variant of a MPK190-v1.3 framework sequence, wherein the one or more framework sequence of MPK190-v1.3 is according to Kabat.

In other embodiments, an antibody or antigen-binding fragment comprises CDRH1, CDRH2, CDRH3, CDRL1, CDRL2, and CDRL3 of MPK190-v1.3, wherein the CDRH1, CDRH2, CDRH3, CDRL1, CDRL2, and CDRL3 are according to Chothia, and the antibody or antigen-binding fragment further comprises one or more framework sequence from MPK190-v1.3 and/or one or more framework sequence that is a variant of a MPK190-v1.3 framework sequence, wherein the one or more framework sequence of MPK190-v1.3 is according to Chothia.

In other embodiments, an antibody or antigen-binding fragment comprises CDRH1, CDRH2, CDRH3, CDRL1, CDRL2, and CDRL3 of MPK190-v1.3, wherein the CDRH1, CDRH2, CDRH3, CDRL1, CDRL2, and CDRL3 are according to Martin (Enhanced Chothia), and the antibody or antigen-binding fragment further comprises one or more framework sequence from MPK190-v1.3 and/or one or more framework sequence that is a variant of a MPK190-v1.3] framework sequence, wherein the one or more framework sequence of MPK190-v1.3 is according to Martin (Enhanced Chothia).

In other embodiments, an antibody or antigen-binding fragment comprises CDRH1, CDRH2, CDRH3, CDRL1, CDRL2, and CDRL3 of MPK190-v1.3, wherein the CDRH1, CDRH2, CDRH3, CDRL1, CDRL2, and CDRL3 are according to AbM, and the antibody or antigen-binding fragment further comprises one or more framework sequence from MPK190-v1.3 and/or one or more framework sequence that is a variant of a MPK190-v1.3 framework sequence, wherein the one or more framework sequence of MPK190-v1.3 is according to AbM.

In other embodiments, an antibody or antigen-binding fragment comprises CDRH1, CDRH2, CDRH3, CDRL1, CDRL2, and CDRL3 of MPK190-v1.3, wherein the CDRH1, CDRH2, CDRH3, CDRL1, CDRL2, and CDRL3 are according to North, and the antibody or antigen-binding fragment further comprises one or more framework sequence from MPK190-v1.3 and/or one or more framework sequence that is a variant of a MPK190-v1.3 framework sequence, wherein the one or more framework sequence of MPK190-v1.3 is according to North.

In other embodiments, an antibody or antigen-binding fragment comprises CDRH1, CDRH2, CDRH3, CDRL1, CDRL2, and CDRL3 of, wherein the CDRH1, CDRH2, CDRH3, CDRL1, CDRL2, and CDRL3 are according to Contact, and the antibody or antigen-binding fragment further comprises one or more framework sequence from MPK190-v1.3 and/or one or more framework sequence that is a variant of a MPK190-v1.3 framework sequence, wherein the one or more framework sequence of MPK190-v1.3 is according to Contact.

In other embodiments, an antibody or antigen-binding fragment comprises CDRH1, CDRH2, CDRH3, CDRL1, CDRL2, and CDRL3 of MPK190-v1.3, wherein the CDRH1, CDRH2, CDRH3, CDRL1, CDRL2, and CDRL3 are according to CCG, and the antibody or antigen-binding fragment further comprises one or more framework sequence from MPK190-v1.3 and/or one or more framework sequence that is a variant of a MPK190-v1.3 framework sequence, wherein the one or more framework sequence of MPK190-v1.3 is according to CCG.

In other embodiments, an antibody or antigen-binding fragment comprises CDRH1, CDRH2, CDRH3, CDRL1, CDRL2, and CDRL3 of MPK190-v1.3, wherein the CDRH1, CDRH2, CDRH3, CDRL1, CDRL2, and CDRL3 are according to EU, and the antibody or antigen-binding fragment further comprises one or more framework sequence from MPK190-v1.3 and/or one or more framework sequence that is a variant of a MPK190-v1.3 framework sequence, wherein the one or more framework sequence of MPK190-v1.3 is according to EU.

In other embodiments, an antibody or antigen-binding fragment comprises CDRH1, CDRH2, CDRH3, CDRL1, CDRL2, and CDRL3 of MPK190-v1.3, wherein the CDRH1, CDRH2, CDRH3, CDRL1, CDRL2, and CDRL3 are according to AHo, and the antibody or antigen-binding fragment further comprises one or more framework sequence from MPK190-v1.3 and/or one or more framework sequence that is a variant of a MPK190-v1.3 framework sequence, wherein the one or more framework sequence of MPK190-v1.3 is according to AHo.

In certain embodiments, an antibody or antigen-binding fragment comprises a VH comprising a FRI, a FR2, a FR3, and/or a FR4 (or a variant of the FR1, FR2, FR3, and/or FR4 comprising one, two, three, four, or five amino acid substitutions (optionally comprising or consisting of one or more conservative substitution), insertions, and/or deletions) of the VH amino acid sequence set forth in SEQ ID NO.: 702 and a VL comprising a FR1, a FR2, a FR3, and/or a FR4 (or a variant of the FRI, FR2, FR3, and/or FR4 comprising one, two, three, four, or five amino acid substitutions (optionally comprising or consisting of one or more conservative substitution), insertions, and/or deletions) of the VL amino acid sequence set forth in SEQ ID NO.: 704. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment comprises a VH comprising a FRI, a FR2, a FR3, and/or a FR4 having at least 80%, at least 85%, at least 90%, at least 91%, at least 92%, at least 93%, at least 94%, at least 95%, at least 96%, at least 97%, at least 98%, or at least 99% itentity to, or comprising, consisting essentially of, or consisting of the respective FR1, FR2, FR3, or FR4 of the VH amino acid sequence set forth in SEQ ID NO.: 702 and a VL comprising a FRI, a FR2, a FR3, and/or a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FRI, FR2, FR3, or FR4 of the of the VL amino acid sequence set forth in SEQ ID NO.: 704. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment comprises a VH comprising a FRI, a FR2, a FR3, and a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FR1, FR2, FR3, or FR4 of the VH amino acid sequence set forth in SEQ ID NO.: 702 and a VL comprising a FRI, a FR2, a FR3, and a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FR1, FR2, FR3, or FR4 of SEQ ID NO.: 704. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment of the present disclosure that binds RSV-F and MPV-F and/or neutralizes RSV and MPV and may comprise one or more VH that binds RSV-F and MPV-F and may further comprise CDRs of a VH sequence according to SEQ ID NO.: 702 and may comprise one or more VL that binds RSV-F and MPV-F and may further comprise CDRs of a VL sequence according to SEQ ID NO.: 704, in which CDRs are as determined using any known CDR numbering method, including the Kabat, Chothia, EU, Martin (Enhanced Chothia), Contact, AbM, CCG, EU, or AHo, numbering method or a combination of two or more of theseIMGT, Martin (Enhanced Chothia), Contact, North, and AHo numbering methods. In certain embodiments, CDRs are according to the IMGT numbering method.

An antibody or antigen-binding fragment of the present disclosure that binds RSV-F and MIPV-F and/or neutralizes RSV and MIPV may comprise a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are determined according to the IMGT numbering system, and wherein: (i) optionally, the CDRH1 comprises or consists of the amino acid sequence set forth in SEQ ID NO.: 121, or a functional variant thereof comprising one, two, or three acid substitutions, one or more of which substitutions is optionally a conservative substitution and/or is a substitution to a germline-encoded amino acid; (ii) optionally, the CDRH2 comprises or consists of the amino acid sequence set forth in SEQ ID NO.: 122, or a functional variant thereof comprising one, two, or three amino acid substitutions, one or more of which substitutions is optionally a conservative substitution and/or is a substitution to a germline-encoded amino acid; (iii) optionally, the CDRH3 comprises or consists of the amino acid sequence set forth in any SEQ ID NO.: 123, or a functional variant thereof comprising one, two, or three amino acid substitutions, one or more of which substitutions is optionally a conservative substitution and/or is a substitution to a germline-encoded amino acid; (iv) optionally, the CDRL1 comprises or consists of the amino acid sequence set forth in SEQ ID NO.: 18, or a functional variant thereof comprising one, two, or three amino acid substitutions, one or more of which substitutions is optionally a conservative substitution and/or is a substitution to a germline-encoded amino acid; (v) optionally, the CDRL2 comprises or consists of the amino acid sequence set forth in SEQ ID NO.: 705, or a functional variant thereof comprising one, two, or three amino acid substitutions, one or more of which substitutions is optionally a conservative substitution and/or is a substitution to a germline-encoded amino acid; and/or (vi) optionally, the CDRL3 comprises or consists of the amino acid sequence set forth in SEQ ID NO.: 127, or a functional variant thereof comprising having one, two, or three amino acid substitutions, one or more of which substitutions is optionally a conservative substitution and/or is a substitution to a germline-encoded amino acid.

In specific embodiments, the antibody or antigen-binding fragment comprises i) a VH and a VL; or a CDRH1, CDRH2, CDRH3, CDRL1, CDRL2, and CDRL3, either as set forth in this “MPK190-v1.3 Antibodies” section or otherwise for a MPK190-v1.3 antibody or as determined by any CDR determination scheme disclosed herein, and ii) a Fc moiety. In certain embodiments, such an antibody or antigen-binding fragment comprises a CH and/or CL (including, potentially a kappa light chain constant region) or a fragment thereof associated with the VH or VL, respectively.

In specific embodiments, the antibody or antigen-binding fragment comprises a VH and a VL, or a CDRH1, CDRH2, CDRH3, CDRL1, CDRL2, and CDRL3, either as set forth in this “MPK190-v1.3 Antibodies” section or otherwise for a MPK190-v1.3 antibody or as determined by any CDR determination scheme disclosed herein, and (A) a Fc moiety that comprises the substitution mutations: (i) G236A, L328V, and Q295E; (ii) G236A, P230A, and Q295E; (iii) G236A, R292P, and 1377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E; (xix) M428L/N434S; (xx) M428L/N434A; (xxi) G236A/A330L/I332E/M428L/N434S; (xxii) G236A/A330L/I332E/M428L/N434A; or (xxiii) any two or more of (i)-(xxii); or (B) an Fc moiety that comprises or consists of a Fc polypeptide or fragment thereof that comprises or consists of amino acid sequences having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs.: 664-700, optionally other than naturally occurring variants thereof, or that comprises or consists of, the amino acid sequences set forth in any one of SEQ ID NOs.: 672-700.

In certain embodiments, the antibody or antigen-binding fragment comprises i) a VH and a VL; or a CDRH1, CDRH2, CDRH3, CDRL1, CDRL2, and CDRL3, either as set forth in this “MPK190-v1.3 Antibodies” section or otherwise for a MPK190-v1.3 antibody or as determined by any CDR determination scheme disclosed herein and ii) an Fc moiety that comprises or consists of a Fc polypeptide or fragment thereof that comprises or consists of amino acid sequences having a sequence according to any one of SEQ ID NOs.: 679-684 and 688-690, or at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequence according to any one of SEQ ID NOs.: 679-684 and 688-690.

In certain embodiments, the antibody or antigen-binding fragment comprises a MPK190-rIG1m3-LS antibody, of which the HC has the amino acid sequence of SEQ ID NO.: 719 and may be encoded by a nucleic acid having the sequence of SEQ ID NO.: 718, and of which the LC has the amino acid sequence of SEQ ID NO.: 721 and may be encoded by a nucleic acid having the sequence of SEQ ID NO.: 720.

In certain embodiments, the antibody or antigen-binding fragment comprises a MPK190-v1.3-rIG1m17,1-LS antibody, of which the HC has the amino acid sequence of SEQ ID NO.: 723 and may be encoded by a nucleic acid having the sequence of SEQ ID NO.: 722, and of which the LC has the amino acid sequence of SEQ ID NO.: 725 and may be encoded by a nucleic acid having the sequence of SEQ ID NO.: 724 or 904.

In certain embodiments, the antibody or antigen-binding fragment comprises a VH and a VL; or a CDRH1, CDRH2, CDRH3, CDRL1, CDRL2, and CDRL3, either as set forth in this “MPK190-v1.3 Antibodies” section or otherwise for a MPK190-v1.3 antibody or as determined by any CDR determination scheme disclosed herein exhibits a synergistic effect in neutralizing or treating and/or preventing infection by RSV when co-administered with at least one of MPK65-v2-v1.2, MPK65-v2-v.3.1, MPK176-v1.3, MPK176-v4.3, MPK201-v1.2, and MPK201-v4.1.

MPK65-v2-v1.2 Antibodies In some embodiments, an antibody or antigen-binding fragment of the present disclosure is a MPK65-v2-v1.2 antibody or antigen-binding fragment therof Such an antibody or antigen-binding fragment thereof may bind and/or neutralize RSV.

In some embodiments, a MPK65-v2-v1.2 antibody may have a VH, a VL, CDRH1-H3, and/or CDRL1-L3 comprising or consisting of amino acid sequences or encoded by polynucleotides having sequences as follows:

| | SEQ ID NOs. | | | |
|---|---|---|---|---|
| | CDRH1-3 | VH | CDRL1-3 | VL |
| Ab | (aa) | (aa/nt) | (aa) | (aa/nt) |
| MPK65-v2-v1.2 | 137, 138, 139 | 136/811 | 142, 143, 852 | 851/850 |

In some embodiments, the VH and VL for a MPK65-v2-v1.2 antibody or antigen-binding fragment that binds RSV-F and/or neutralizes RSV comprise or consist of a VH and VL having the sequences of SEQ ID NOs.: 136 and 851, respectively.

In certain embodiments, an antibody or antigen-binding fragment of the present disclosure binds RSV-F and/or neutralizes RSV and:
(i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in SEQ ID NO.: 136, wherein sequence variation is optionally limited to one or more framework regions and/or sequence variation comprises one or more substitution to a germline-encoded amino acid; and/or
(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in SEQ ID NO.: 851, wherein sequence variation is optionally limited to one or more framework regions and/or sequence variation comprises one or more substitution to a germline-encoded amino acid.

In some embodiments, variation as compared to SEQ ID NO.: 136 or SEQ ID NO.: 851 is limited to one or more framework region. In some embodiments, the variation comprises or consists of one, two, three, four, five, six, seven, eight, nine, or ten amino acid substitutions (any or all of which may be a conservative substitution), insertions, or deletions in the VH, in the VL, or in both. In some embodiments, the variation comprises one or more amino acid substitution, insertion, and/or deletion of an amino acid that is two, three, four, five, six, seven, eight, nine, ten, eleven, twelve, thirteen, fourteen, fifteen, or more amino acid positions away from an N-terminal and/or a C-terminal amino acid of a CDR.

Framework regions can be identified according to a numbering scheme (e.g., IMGT, Kabat, Chothia, North, EU, Martin (Enhanced Chothia), Contact, AbM, CCG, EU, or AHo, or a combination of two or more of these). The CDRs may be identified within a variable domain or within a heavy or light chain according to a numbering scheme or a combination of numbering schemes, and, preferably, the FRs may be identified using the same numbering scheme or combination of numbering schemes.

For example, in some embodiments, an antibody or antigen-binding fragment comprises CDRH1, CDRH2, CDRH3, CDRL1, CDRL2, and CDRL3 of MPK65-v2-v1.2, wherein the CDRH1, CDRH2, CDRH3, CDRL1, CDRL2, and CDRL3 are according to IMGT (optionally IMGT-junction for CDRH3 and CDRL3), and the antibody or antigen-binding fragment further comprises one or more framework sequence from MPK65-v2-v1.2 and/or one or more framework sequence that is a variant of a MPK65-v2-v1.2framework sequence, wherein the one or more framework sequence of MPK65-v2-v1.2 is according to IMGT (and if IMGT-junction is used for CDRH3 and CDRL3, accounting for this).

In other embodiments, an antibody or antigen-binding fragment comprises CDRH1, CDRH2, CDRH3, CDRL1, CDRL2, and CDRL3 of MPK65-v2-v1.2, wherein the CDRH1, CDRH2, CDRH3, CDRL1, CDRL2, and CDRL3 are according to Kabat, and the antibody or antigen-binding fragment further comprises one or more framework sequence from MPK65-v2-v1.2 and/or one or more framework sequence that is a variant of a MPK65-v2-v1.2 framework sequence, wherein the one or more framework sequence of MPK65-v2-v1.2 is according to Kabat.

In other embodiments, an antibody or antigen-binding fragment comprises CDRH1, CDRH2, CDRH3, CDRL1, CDRL2, and CDRL3 of MPK65-v2-v1.2, wherein the CDRH1, CDRH2, CDRH3, CDRL1, CDRL2, and CDRL3 are according to Chothia, and the antibody or antigen-binding fragment further comprises one or more framework sequence from MPK65-v2-v1.2 and/or one or more framework sequence that is a variant of a MPK65-v2-v1.2 framework sequence, wherein the one or more framework sequence of MPK65-v2-v1.2 is according to Chothia.

In other embodiments, an antibody or antigen-binding fragment comprises CDRH1, CDRH2, CDRH3, CDRL1, CDRL2, and CDRL3 of MPK65-v2-v1.2, wherein the CDRH1, CDRH2, CDRH3, CDRL1, CDRL2, and CDRL3 are according to Martin (Enhanced Chothia), and the antibody or antigen-binding fragment further comprises one or more framework sequence from MPK65-v2-v1.2 and/or one or more framework sequence that is a variant of a MPK65-v2-v1.2 framework sequence, wherein the one or more framework sequence of MPK65-v2-v1.2 is according to Martin (Enhanced Chothia).

In other embodiments, an antibody or antigen-binding fragment comprises CDRH1, CDRH2, CDRH3, CDRL1, CDRL2, and CDRL3 of MPK65-v2-v1.2, wherein the CDRH1, CDRH2, CDRH3, CDRL1, CDRL2, and CDRL3 are according to AbM, and the antibody or antigen-binding fragment further comprises one or more framework sequence from MPK65-v2-v1.2 and/or one or more framework sequence that is a variant of a MPK65-v2-v1.2 framework sequence, wherein the one or more framework sequence of MPK65-v2-v1.2 is according to AbM.

In other embodiments, an antibody or antigen-binding fragment comprises CDRH1, CDRH2, CDRH3, CDRL1, CDRL2, and CDRL3 of MPK65-v2-v1.2, wherein the CDRH1, CDRH2, CDRH3, CDRL1, CDRL2, and CDRL3 are according to North, and the antibody or antigen-binding fragment further comprises one or more framework sequence from MPK65-v2-v1.2 and/or one or more framework sequence that is a variant of a MPK65-v2-v1.2 framework sequence, wherein the one or more framework sequence of MPK65-v2-v1.2 is according to North.

In other embodiments, an antibody or antigen-binding fragment comprises CDRH1, CDRH2, CDRH3, CDRL1, CDRL2, and CDRL3 of, wherein the CDRH1, CDRH2, CDRH3, CDRL1, CDRL2, and CDRL3 are according to Contact, and the antibody or antigen-binding fragment further comprises one or more framework sequence from MPK65-v2-v1.2 and/or one or more framework sequence that is a variant of a MPK65-v2-v1.2 framework sequence, wherein the one or more framework sequence of MPK65-v2-v1.2 is according to Contact.

In other embodiments, an antibody or antigen-binding fragment comprises CDRH1, CDRH2, CDRH3, CDRL1, CDRL2, and CDRL3 of MPK65-v2-v1.2, wherein the CDRH1, CDRH2, CDRH3, CDRL1, CDRL2, and CDRL3 are according to CCG, and the antibody or antigen-binding fragment further comprises one or more framework sequence from MPK65-v2-v1.2 and/or one or more framework sequence that is a variant of a MPK65-v2-v1.2 framework sequence, wherein the one or more framework sequence of MPK65-v2-v1.2 is according to CCG.

In other embodiments, an antibody or antigen-binding fragment comprises CDRH1, CDRH2, CDRH3, CDRL1, CDRL2, and CDRL3 of MPK65-v2-v1.2, wherein the CDRH1, CDRH2, CDRH3, CDRL1, CDRL2, and CDRL3 are according to EU, and the antibody or antigen-binding fragment further comprises one or more framework sequence from MPK65-v2-v1.2 and/or one or more framework sequence that is a variant of a MPK65-v2-v1.2 framework sequence, wherein the one or more framework sequence of MPK65-v2-v1.2 is according to EU.

In other embodiments, an antibody or antigen-binding fragment comprises CDRH1, CDRH2, CDRH3, CDRL1, CDRL2, and CDRL3 of MPK65-v2-v1.2, wherein the CDRH1, CDRH2, CDRH3, CDRL1, CDRL2, and CDRL3 are according to AHo, and the antibody or antigen-binding fragment further comprises one or more framework sequence from MPK65-v2-v1.2 and/or one or more framework sequence that is a variant of a MPK65-v2-v1.2 framework sequence, wherein the one or more framework sequence of MPK65-v2-v1.2 is according to AHo.

In certain embodiments, an antibody or antigen-binding fragment comprises a VH comprising a FRI, a FR2, a FR3, and/or a FR4 (or a variant of the FR1, FR2, FR3, and/or FR4 comprising one, two, three, four, or five amino acid substitutions (optionally comprising or consisting of one or more conservative substitution), insertions, and/or deletions) of the VH amino acid sequence set forth in SEQ ID NO.: 136 and a VL comprising a FR1, a FR2, a FR3, and/or a FR4 (or a variant of the FRI, FR2, FR3, and/or FR4 comprising one, two, three, four, or five amino acid substitutions (optionally comprising or consisting of one or more conservative substitution), insertions, and/or deletions) of the VL amino acid sequence set forth in SEQ ID NO.: 851. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment comprises a VH comprising a FRI, a FR2, a FR3, and/or a FR4 having at least 80%, at least 85%, at least 90%, at least 91%, at least 92%, at least 93%, at least 94%, at least 95%, at least 96%, at least 97%, at least 98%, or at least 99% itentity to, or comprising, consisting essentially of, or consisting of the respective FR1, FR2, FR3, or FR4 of the VH amino acid sequence set forth in SEQ ID NO.: 136 and a VL comprising a FRI, a FR2, a FR3, and/or a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FRI, FR2, FR3, or FR4 of the of the VL amino acid sequence set forth in SEQ ID NO.: 851. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment comprises a VH comprising a FRI, a FR2, a FR3, and a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FR1, FR2, FR3, or FR4 of the VH amino acid sequence set forth in SEQ ID NO.: 136 and a VL comprising a FRI, a FR2, a FR3, and a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FR1, FR2, FR3, or FR4 of SEQ ID NO.: 851. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment of the present disclosure that binds RSV-F and/or neutralizes RSV and may comprise one or more VH that binds RSV-F and may further comprise CDRs of a VH sequence according to SEQ ID NO.: 136 and may comprise one or more VL that binds RSV-F and may further comprise CDRs of a VL sequence according to SEQ ID NO.: 851, in which CDRs are as determined using any known CDR numbering method, including the Kabat, Chothia, EU, Martin (Enhanced Chothia), Contact, AbM, CCG, EU, or AHo, numbering method or a combination of two or more of theseIMGT, Martin (Enhanced Chothia), Contact, North, and AHo numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software. In certain embodiments, CDRs are according to the Kabat numbering method. In certain embodiments, CDRs are according to the AHo numbering method. In certain embodiments, CDRs are according to the North numbering method.

An antibody or antigen-binding fragment of the present disclosure that binds RSV-F and/or neutralizes RSV may comprise a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are determined according to the IMGT numbering system, and wherein: (i) optionally, the CDRH1 comprises or consists of the amino acid sequence set forth in SEQ ID NO.: 137, or a functional variant thereof comprising one, two, or three acid substitutions, one or more of which substitutions is optionally a conservative substitution and/or is a substitution to a germline-encoded amino acid; (ii) optionally, the CDRH2 comprises or consists of the amino acid sequence set forth in SEQ ID NO.: 138, or a functional variant thereof comprising one, two, or three amino acid substitutions, one or more of which substitutions is optionally a conservative substitution and/or is a substitution to a germline-encoded amino acid; (iii) optionally, the CDRH3 comprises or consists of the amino acid sequence set forth in any SEQ ID NO.: 139, or a functional variant thereof comprising one, two, or three amino acid substitutions, one or more of which substitutions is optionally a conservative substitution and/or is a substitution to a germline-encoded amino acid; (iv) optionally, the CDRL1 comprises or consists of the amino acid sequence set forth in SEQ ID NO.: 142, or a functional variant thereof comprising one, two, or three amino acid substitutions, one or more of which substitutions is optionally a conservative substitution and/or is a substitution to a germline-encoded amino acid; (v) optionally, the CDRL2 comprises or consists of the amino acid sequence set forth in SEQ ID NO.: 143, or a functional variant thereof comprising one, two, or three amino acid substitutions, one or more of which substitutions is optionally a conservative substitution and/or is a substitution to a germline-encoded amino acid; and/or (vi) optionally, the CDRL3 comprises or consists of the amino acid sequence set forth in SEQ ID NO.: 852, or a functional variant thereof comprising having one, two, or three amino acid substitutions, one or more of which substitutions is optionally a conservative substitution and/or is a substitution to a germline-encoded amino acid.

In specific embodiments, the antibody or antigen-binding fragment comprises i) a VH and a VL; or a CDRH1, CDRH2, CDRH3, CDRL1, CDRL2, and CDRL3, either as set forth in this “MPK65-v2-v1.2 Antibodies” section or otherwise for a MPK65-v2-v1.2 antibody or as determined by any CDR determination scheme disclosed herein, and ii) a Fc moiety. In certain embodiments, such an antibody or antigen-binding fragment comprises a CH and/or CL (including, potentially a kappa light chain constant region) or a fragment thereof associated with the VH or VL, respectively.

In specific embodiments, the antibody or antigen-binding fragment comprises a VH and a VL; or a CDRH1, CDRH2, CDRH3, CDRL1, CDRL2, and CDRL3, either as set forth in this “MPK65-v2-v1.2 Antibodies” section or otherwise for a MPK65-v2-v1.2 antibody or as determined by any CDR determination scheme disclosed herein, and (A) a Fc moiety that comprises the substitution mutations: (i) G236A, L328V, and Q295E; (ii) G236A, P230A, and Q295E; (iii) G236A, R292P, and 1377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E; (xix) M428L/N434S; (xx) M428L/N434A; (xxi) G236A/A330L/1332E/M428L/N434S; (xxii) G236A/A330L/1332E/M428L/N434A; or (xxiii) any two or more of (i)-(xxii); or (B) an Fc moiety that comprises or consists of a Fc polypeptide or fragment thereof that comprises or consists of amino acid sequences having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs.: 664-700, optionally other than naturally occurring variants thereof, or that comprises or consists of, the amino acid sequences set forth in any one of SEQ ID NOs.: 672-700.

In certain embodiments, the antibody or antigen-binding fragment comprises i) a VH and a VL; or a CDRH1, CDRH2, CDRH3, CDRL1, CDRL2, and CDRL3, either as set forth in this “MPK65-v2-v1.2 Antibodies” section or otherwise for a MPK65-v2-v1.2 antibody or as determined by any CDR determination scheme disclosed herein and ii) an Fc moiety that comprises or consists of a Fc polypeptide or fragment thereof that comprises or consists of amino acid sequences having a sequence according to any one of SEQ ID NOs.: 679-684 and 688-690, or at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequence according to any one of SEQ ID NOs.: 679-684 and 688-690.

In certain embodiments, the antibody or antigen-binding fragment comprises a MPK65-v2-v1.2-rIG1m3-LS antibody.

In certain embodiments, the antibody or antigen-binding fragment comprises a MPK65-v2-v1.2-rIG1m17,1-LS antibody.

MPK65-v2-v3.1 Antibodies In some embodiments, an antibody or antigen-binding fragment of the present disclosure is a MPK65-v2-v3.1 antibody or antigen-binding fragment therof Such an antibody or antigen-binding fragment thereof may bind and/or neutralize RSV.

In some embodiments, a MPK65-v2-v3.1 antibody may have a VH, a VL, CDRH1-H3, and/or CDRL1-L3 comprising or consisting of amino acid sequences or encoded by polynucleotides having sequences as follows:

| | SEQ ID NOs. | | | |
|---|---|---|---|---|
| | CDRH1-3 | VH | CDRL1-3 | VL |
| Ab | (aa) | (aa/nt) | (aa) | (aa/nt) |
| MPK65-v2-v3.1 | 814, 818, 139 | 817/816 | 142, 143, 144 | 141/849 |

In some embodiments, the VH and VL for a MPK65-v2-v3.1 antibody or antigen-binding fragment that binds RSV-F and/or neutralizes RSV comprise or consist of a VH and VL having the sequences of SEQ ID NOs.: 817 and 141, respectively.

In certain embodiments, an antibody or antigen-binding fragment of the present disclosure binds RSV-F and/or neutralizes RSV and:
(i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in SEQ ID NO.: 817, wherein sequence variation is optionally limited to one or more framework regions and/or sequence variation comprises one or more substitution to a germline-encoded amino acid; and/or
(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in SEQ ID NO.: 141, wherein sequence variation is optionally limited to one or more framework regions and/or sequence variation comprises one or more substitution to a germline-encoded amino acid.

In some embodiments, variation as compared to SEQ ID NO.: 817 or SEQ ID NO.: 141 is limited to one or more framework region. In some embodiments, the variation comprises or consists of one, two, three, four, five, six, seven, eight, nine, or ten amino acid substitutions (any or all of which may be a conservative substitution), insertions, or deletions in the VH, in the VL, or in both. In some embodiments, the variation comprises one or more amino acid substitution, insertion, and/or deletion of an amino acid that is two, three, four, five, six, seven, eight, nine, ten, eleven, twelve, thirteen, fourteen, fifteen, or more amino acid positions away from an N-terminal and/or a C-terminal amino acid of a CDR.

Framework regions can be identified according to a numbering scheme (e.g., IMGT, Kabat, Chothia, North, EU, Martin (Enhanced Chothia), Contact, AbM, CCG, EU, or AHo, or a combination of two or more of these). The CDRs may be identified within a variable domain or within a heavy or light chain according to a numbering scheme or a combination of numbering schemes, and, preferably, the FRs may be identified using the same numbering scheme or combination of numbering schemes.

For example, in some embodiments, an antibody or antigen-binding fragment comprises CDRH1, CDRH2, CDRH3, CDRL1, CDRL2, and CDRL3 of MPK65-v2-v3.1, wherein the CDRH1, CDRH2, CDRH3, CDRL1, CDRL2, and CDRL3 are according to IMGT (optionally IMGT-junction for CDRH3 and CDRL3), and the antibody or antigen-binding fragment further comprises one or more framework sequence from MPK65-v2-v3.1 and/or one or more framework sequence that is a variant of a MPK65-v2-v3.1framework sequence, wherein the one or more framework sequence of MPK65-v2-v3.1 is according to IMGT (and if IMGT-junction is used for CDRH3 and CDRL3, accounting for this).

In other embodiments, an antibody or antigen-binding fragment comprises CDRH1, CDRH2, CDRH3, CDRL1, CDRL2, and CDRL3 of MPK65-v2-v3.1, wherein the CDRH1, CDRH2, CDRH3, CDRL1, CDRL2, and CDRL3 are according to Kabat, and the antibody or antigen-binding fragment further comprises one or more framework sequence from MPK65-v2-v3.1 and/or one or more framework sequence that is a variant of a MPK65-v2-v3.1 framework sequence, wherein the one or more framework sequence of MPK65-v2-v3.1 is according to Kabat.

In other embodiments, an antibody or antigen-binding fragment comprises CDRH1, CDRH2, CDRH3, CDRL1, CDRL2, and CDRL3 of MPK65-v2-v3.1, wherein the CDRH1, CDRH2, CDRH3, CDRL1, CDRL2, and CDRL3 are according to Chothia, and the antibody or antigen-binding fragment further comprises one or more framework sequence from MPK65-v2-v3.1 and/or one or more framework sequence that is a variant of a MPK65-v2-v3.1 framework sequence, wherein the one or more framework sequence of MPK65-v2-v3.1 is according to Chothia.

In other embodiments, an antibody or antigen-binding fragment comprises CDRH1, CDRH2, CDRH3, CDRL1, CDRL2, and CDRL3 of MPK65-v2-v3.1, wherein the CDRH1, CDRH2, CDRH3, CDRL1, CDRL2, and CDRL3 are according to Martin (Enhanced Chothia), and the antibody or antigen-binding fragment further comprises one or more framework sequence from MPK65-v2-v3.1 and/or one or more framework sequence that is a variant of a MPK65-v2-v3.1 framework sequence, wherein the one or more framework sequence of MPK65-v2-v3.1 is according to Martin (Enhanced Chothia).

In other embodiments, an antibody or antigen-binding fragment comprises CDRH1, CDRH2, CDRH3, CDRL1, CDRL2, and CDRL3 of MPK65-v2-v3.1, wherein the CDRH1, CDRH2, CDRH3, CDRL1, CDRL2, and CDRL3 are according to AbM, and the antibody or antigen-binding fragment further comprises one or more framework sequence from MPK65-v2-v3.1 and/or one or more framework sequence that is a variant of a MPK65-v2-v3.1 framework sequence, wherein the one or more framework sequence of MPK65-v2-v3.1 is according to AbM.

In other embodiments, an antibody or antigen-binding fragment comprises CDRH1, CDRH2, CDRH3, CDRL1, CDRL2, and CDRL3 of MPK65-v2-v3.1, wherein the CDRH1, CDRH2, CDRH3, CDRL1, CDRL2, and CDRL3 are according to North, and the antibody or antigen-binding fragment further comprises one or more framework sequence from MPK65-v2-v3.1 and/or one or more framework sequence that is a variant of a MPK65-v2-v3.1 framework sequence, wherein the one or more framework sequence of MPK65-v2-v3.1 is according to North.

In other embodiments, an antibody or antigen-binding fragment comprises CDRH1, CDRH2, CDRH3, CDRL1, CDRL2, and CDRL3 of, wherein the CDRH1, CDRH2, CDRH3, CDRL1, CDRL2, and CDRL3 are according to Contact, and the antibody or antigen-binding fragment further comprises one or more framework sequence from MPK65-v2-v3.1 and/or one or more framework sequence that is a variant of a MPK65-v2-v3.1 framework sequence, wherein the one or more framework sequence of MPK65-v2-v3.1 is according to Contact.

In other embodiments, an antibody or antigen-binding fragment comprises CDRH1, CDRH2, CDRH3, CDRL1, CDRL2, and CDRL3 of MPK65-v2-v3.1, wherein the CDRH1, CDRH2, CDRH3, CDRL1, CDRL2, and CDRL3 are according to CCG, and the antibody or antigen-binding fragment further comprises one or more framework sequence from MPK65-v2-v3.1 and/or one or more framework sequence that is a variant of a MPK65-v2-v3.1 framework sequence, wherein the one or more framework sequence of MPK65-v2-v3.1 is according to CCG.

In other embodiments, an antibody or antigen-binding fragment comprises CDRH1, CDRH2, CDRH3, CDRL1, CDRL2, and CDRL3 of MPK65-v2-v3.1, wherein the CDRH1, CDRH2, CDRH3, CDRL1, CDRL2, and CDRL3 are according to EU, and the antibody or antigen-binding fragment further comprises one or more framework sequence from MPK65-v2-v3.1 and/or one or more framework sequence that is a variant of a MPK65-v2-v3.1 framework sequence, wherein the one or more framework sequence of MPK65-v2-v3.1 is according to EU.

In other embodiments, an antibody or antigen-binding fragment comprises CDRH1, CDRH2, CDRH3, CDRL1, CDRL2, and CDRL3 of MPK65-v2-v3.1, wherein the CDRH1, CDRH2, CDRH3, CDRL1, CDRL2, and CDRL3 are according to AHo, and the antibody or antigen-binding fragment further comprises one or more framework sequence from MPK65-v2-v3.1 and/or one or more framework sequence that is a variant of a MPK65-v2-v3.1 framework sequence, wherein the one or more framework sequence of MPK65-v2-v3.1 is according to AHo.

In certain embodiments, an antibody or antigen-binding fragment comprises a VH comprising a FRI, a FR2, a FR3, and/or a FR4 (or a variant of the FR1, FR2, FR3, and/or FR4 comprising one, two, three, four, or five amino acid substitutions (optionally comprising or consisting of one or more conservative substitution), insertions, and/or deletions) of the VH amino acid sequence set forth in SEQ ID NO.: 817 and a VL comprising a FR1, a FR2, a FR3, and/or a FR4 (or a variant of the FRI, FR2, FR3, and/or FR4 comprising one, two, three, four, or five amino acid substitutions (optionally comprising or consisting of one or more conservative substitution), insertions, and/or deletions) of the VL amino acid sequence set forth in SEQ ID NO.: 141. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment comprises a VH comprising a FRI, a FR2, a FR3, and/or a FR4 having at least 80%, at least 85%, at least 90%, at least 91%, at least 92%, at least 93%, at least 94%, at least 95%, at least 96%, at least 97%, at least 98%, or at least 99% itentity to, or comprising, consisting essentially of, or consisting of the respective FR1, FR2, FR3, or FR4 of the VH amino acid sequence set forth in SEQ ID NO.: 817 and a VL comprising a FRI, a FR2, a FR3, and/or a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FRI, FR2, FR3, or FR4 of the of the VL amino acid sequence set forth in SEQ ID NO.: 141. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment comprises a VH comprising a FRI, a FR2, a FR3, and a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FR1, FR2, FR3, or FR4 of the VH amino acid sequence set forth in SEQ ID NO.: 817 and a VL comprising a FRI, a FR2, a FR3, and a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FR1, FR2, FR3, or FR4 of SEQ ID NO.: 141. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment of the present disclosure that binds RSV-F and/or neutralizes RSV and may comprise one or more VH that binds RSV-F and may further comprise CDRs of a VH sequence according to SEQ ID NO.: 817 and may comprise one or more VL that binds RSV-F and may further comprise CDRs of a VL sequence according to SEQ ID NO.: 141, in which CDRs are as determined using any known CDR numbering method, including the Kabat, Chothia, EU, Martin (Enhanced Chothia), Contact, AbM, CCG, EU, or AHo, numbering method or a combination of two or more of theseIMGT, Martin (Enhanced Chothia), Contact, North, and AHo numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software. In certain embodiments, CDRs are according to the Kabat numbering method. In certain embodiments, CDRs are according to the AHo numbering method. In certain embodiments, CDRs are according to the North numbering method.

An antibody or antigen-binding fragment of the present disclosure that binds RSV-F and/or neutralizes RSV may comprise a heavy chain variable domain (VH) comprising a complementarity determining region (CDR)H1, a CDRH2, and a CDRH3, and a light chain variable domain (VL) comprising a CDRL 1, a CDRL2, and a CDRL3, wherein the CDRs are determined according to the IMGT numbering system, and wherein: (i) optionally, the CDRH1 comprises or consists of the amino acid sequence set forth in SEQ ID NO.: 814, or a functional variant thereof comprising one, two, or three acid substitutions, one or more of which substitutions is optionally a conservative substitution and/or is a substitution to a germline-encoded amino acid; (ii) optionally, the CDRH2 comprises or consists of the amino acid sequence set forth in SEQ ID NO.: 818, or a functional variant thereof comprising one, two, or three amino acid substitutions, one or more of which substitutions is optionally a conservative substitution and/or is a substitution to a germline-encoded amino acid; (iii) optionally, the CDRH3 comprises or consists of the amino acid sequence set forth in any SEQ ID NO.: 139, or a functional variant thereof comprising one, two, or three amino acid substitutions, one or more of which substitutions is optionally a conservative substitution and/or is a substitution to a germline-encoded amino acid; (iv) optionally, the CDRL1 comprises or consists of the amino acid sequence set forth in SEQ ID NO.: 142, or a functional variant thereof comprising one, two, or three amino acid substitutions, one or more of which substitutions is optionally a conservative substitution and/or is a substitution to a germline-encoded amino acid; (v) optionally, the CDRL2 comprises or consists of the amino acid sequence set forth in SEQ ID NO.: 143, or a functional variant thereof comprising one, two, or three amino acid substitutions, one or more of which substitutions is optionally a conservative substitution and/or is a substitution to a germline-encoded amino acid; and/or (vi) optionally, the CDRL3 comprises or consists of the amino acid sequence set forth in SEQ ID NO.: 144, or a functional variant thereof comprising having one, two, or three amino acid substitutions, one or more of which substitutions is optionally a conservative substitution and/or is a substitution to a germline-encoded amino acid.

In specific embodiments, the antibody or antigen-binding fragment comprises i) a VH and a VL; or a CDRH1, CDRH2, CDRH3, CDRL1, CDRL2, and CDRL3, either as set forth in this “MPK65-v2-v3.1 Antibodies” section or otherwise for a MPK65-v2-v3.1 antibody or as determined by any CDR determination scheme disclosed herein, and ii) a Fc moiety. In certain embodiments, such an antibody or antigen-binding fragment comprises a CH and/or CL (including, potentially a kappa light chain constant region) or a fragment thereof associated with the VH or VL, respectively.

In specific embodiments, the antibody or antigen-binding fragment comprises a VH and a VL; or a CDRH1, CDRH2, CDRH3, CDRL1, CDRL2, and CDRL3, either as set forth in this “MPK65-v2-v3.1 Antibodies” section or otherwise for a MPK65-v2-v3.1 antibody or as determined by any CDR determination scheme disclosed herein, and (A) a Fc moiety that comprises the substitution mutations: (i) G236A, L328V, and Q295E; (ii) G236A, P230A, and Q295E; (iii) G236A, R292P, and 1377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E; (xix) M428L/N434S; (xx) M428L/N434A; (xxi) G236A/A330L/I332E/M428L/N434S; (xxii) G236A/A330L/I332E/M428L/N434A; or (xxiii) any two or more of (i)-(xxii); or (B) an Fc moiety that comprises or consists of a Fc polypeptide or fragment thereof that comprises or consists of amino acid sequences having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs.: 664-700, optionally other than naturally occurring variants thereof, or that comprises or consists of, the amino acid sequences set forth in any one of SEQ ID NOs.: 672-700.

In certain embodiments, the antibody or antigen-binding fragment comprises i) a VH and a VL; or a CDRH1, CDRH2, CDRH3, CDRL1, CDRL2, and CDRL3, either as set forth in this “MPK65-v2-v3.1 Antibodies” section or otherwise for a MPK65-v2-v3.1 antibody or as determined by any CDR determination scheme disclosed herein and ii) an Fc moiety that comprises or consists of a Fc polypeptide or fragment thereof that comprises or consists of amino acid sequences having a sequence according to any one of SEQ ID NOs.: 679-684 and 688-690, or at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequence according to any one of SEQ ID NOs.: 679-684 and 688-690.

In certain embodiments, the antibody or antigen-binding fragment comprises a MPK65-v2-v3.1-rIG1m3-LS antibody.

In certain embodiments, the antibody or antigen-binding fragment comprises a MPK65-v2-v3.1-rIG1m17,1-LS antibody.

MPK176-v1.3 Antibodies In some embodiments, an antibody or antigen-binding fragment of the present disclosure is a MPK176-v1.3 antibody or antigen-binding fragment therof. Such an antibody or antigen-binding fragment thereof may bind RSV and/or neutralize RSV.

In some embodiments, a MPK176-v1.3 antibody may have a VH, a VL, CDRH1-H3, and/or CDRL1-L3 comprising or consisting of amino acid sequences or encoded by polynucleotides having sequences as follows:

| | SEQ ID NOs. | | | |
|---|---|---|---|---|
| Ab | CDRH1-3 (aa) | VH (aa/nt) | CDRL1-3 (aa) | VL (aa/nt) |
| MPK176-v1.3 | 234, 235, 236 | 233/829 | 239, 240, 241 | 858/857 |

In some embodiments, the VH and VL for a MPK176-v1.3 antibody or antigen-binding fragment that binds RSV-F and/or neutralizes RSV comprise or consist of a VH and VL having the sequences of SEQ ID NOs.: 233 and 858, respectively.

In certain embodiments, an antibody or antigen-binding fragment of the present disclosure binds RSV-F and/or neutralizes RSV and:
(i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in SEQ ID NO.: 233, wherein sequence variation is optionally limited to one or more framework regions and/or sequence variation comprises one or more substitution to a germline-encoded amino acid; and/or
(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in SEQ ID NO.: 858, wherein sequence variation is optionally limited to one or more framework regions and/or sequence variation comprises one or more substitution to a germline-encoded amino acid.

In some embodiments, variation as compared to SEQ ID NO.: 233 or SEQ ID NO.: 858 is limited to one or more framework region. In some embodiments, the variation comprises or consists of one, two, three, four, five, six, seven, eight, nine, or ten amino acid substitutions (any or all of which may be a conservative substitution), insertions, or deletions in the VH, in the VL, or in both. In some embodiments, the variation comprises one or more amino acid substitution, insertion, and/or deletion of an amino acid that is two, three, four, five, six, seven, eight, nine, ten, eleven, twelve, thirteen, fourteen, fifteen, or more amino acid positions away from an N-terminal and/or a C-terminal amino acid of a CDR.

Framework regions can be identified according to a numbering scheme (e.g., IMGT, Kabat, Chothia, North, EU, Martin (Enhanced Chothia), Contact, AbM, CCG, EU, or AHo, or a combination of two or more of these). The CDRs may be identified within a variable domain or within a heavy or light chain according to a numbering scheme or a combination of numbering schemes, and, preferably, the FRs may be identified using the same numbering scheme or combination of numbering schemes.

For example, in some embodiments, an antibody or antigen-binding fragment comprises CDRH1, CDRH2, CDRH3, CDRL1, CDRL2, and CDRL3 of MPK176-v1.3, wherein the CDRH1, CDRH2, CDRH3, CDRL1, CDRL2, and CDRL3 are according to IMGT (optionally IMGT-junction for CDRH3 and CDRL3), and the antibody or antigen-binding fragment further comprises one or more framework sequence from MPK176-v1.3 and/or one or more framework sequence that is a variant of a MPK176-v1.3framework sequence, wherein the one or more framework sequence of MPK176-v1.3 is according to IMGT (and if IMGT-junction is used for CDRH3 and CDRL3, accounting for this).

In other embodiments, an antibody or antigen-binding fragment comprises CDRH1, CDRH2, CDRH3, CDRL1, CDRL2, and CDRL3 of MPK176-v1.3, wherein the CDRH1, CDRH2, CDRH3, CDRL1, CDRL2, and CDRL3 are according to Kabat, and the antibody or antigen-binding fragment further comprises one or more framework sequence from MPK176-v1.3 and/or one or more framework sequence that is a variant of a MPK176-v1.3 framework sequence, wherein the one or more framework sequence of MPK176-v1.3 is according to Kabat.

In other embodiments, an antibody or antigen-binding fragment comprises CDRH1, CDRH2, CDRH3, CDRL1, CDRL2, and CDRL3 of MPK176-v1.3, wherein the CDRH1, CDRH2, CDRH3, CDRL1, CDRL2, and CDRL3 are according to Chothia, and the antibody or antigen-binding fragment further comprises one or more framework sequence from MPK176-v1.3 and/or one or more framework sequence that is a variant of a MPK176-v1.3 framework sequence, wherein the one or more framework sequence of MPK176-v1.3 is according to Chothia.

In other embodiments, an antibody or antigen-binding fragment comprises CDRH1, CDRH2, CDRH3, CDRL1, CDRL2, and CDRL3 of MPK176-v1.3, wherein the CDRH1, CDRH2, CDRH3, CDRL1, CDRL2, and CDRL3 are according to Martin (Enhanced Chothia), and the antibody or antigen-binding fragment further comprises one or more framework sequence from MPK176-v1.3 and/or one or more framework sequence that is a variant of a MPK176-v1.3 framework sequence, wherein the one or more framework sequence of MPK176-v1.3 is according to Martin (Enhanced Chothia).

In other embodiments, an antibody or antigen-binding fragment comprises CDRH1, CDRH2, CDRH3, CDRL1, CDRL2, and CDRL3 of MPK176-v1.3, wherein the CDRH1, CDRH2, CDRH3, CDRL1, CDRL2, and CDRL3 are according to AbM, and the antibody or antigen-binding fragment further comprises one or more framework sequence from MPK176-v1.3 and/or one or more framework sequence that is a variant of a MPK176-v1.3 framework sequence, wherein the one or more framework sequence of MPK176-v1.3 is according to AbM.

In other embodiments, an antibody or antigen-binding fragment comprises CDRH1, CDRH2, CDRH3, CDRL1, CDRL2, and CDRL3 of MPK176-v1.3, wherein the CDRH1, CDRH2, CDRH3, CDRL1, CDRL2, and CDRL3 are according to North, and the antibody or antigen-binding fragment further comprises one or more framework sequence from MPK176-v1.3 and/or one or more framework sequence that is a variant of a MPK176-v1.3 framework sequence, wherein the one or more framework sequence of MPK176-v1.3 is according to North.

In other embodiments, an antibody or antigen-binding fragment comprises CDRH1, CDRH2, CDRH3, CDRL1, CDRL2, and CDRL3 of, wherein the CDRH1, CDRH2, CDRH3, CDRL1, CDRL2, and CDRL3 are according to Contact, and the antibody or antigen-binding fragment further comprises one or more framework sequence from MPK176-v1.3 and/or one or more framework sequence that is a variant of a MPK176-v1.3 framework sequence, wherein the one or more framework sequence of MPK176-v1.3 is according to Contact.

In other embodiments, an antibody or antigen-binding fragment comprises CDRH1, CDRH2, CDRH3, CDRL1, CDRL2, and CDRL3 of MPK176-v1.3, wherein the CDRH1, CDRH2, CDRH3, CDRL1, CDRL2, and CDRL3 are according to CCG, and the antibody or antigen-binding fragment further comprises one or more framework sequence from MPK176-v1.3 and/or one or more framework sequence that is a variant of a MPK176-v1.3 framework sequence, wherein the one or more framework sequence of MPK176-v1.3 is according to CCG.

In other embodiments, an antibody or antigen-binding fragment comprises CDRH1, CDRH2, CDRH3, CDRL1, CDRL2, and CDRL3 of MPK176-v1.3, wherein the CDRH1, CDRH2, CDRH3, CDRL1, CDRL2, and CDRL3 are according to EU, and the antibody or antigen-binding fragment further comprises one or more framework sequence from MPK176-v1.3 and/or one or more framework sequence that is a variant of a MPK176-v1.3 framework sequence, wherein the one or more framework sequence of MPK176-v1.3 is according to EU.

In other embodiments, an antibody or antigen-binding fragment comprises CDRH1, CDRH2, CDRH3, CDRL1, CDRL2, and CDRL3 of MPK176-v1.3, wherein the CDRH1, CDRH2, CDRH3, CDRL1, CDRL2, and CDRL3 are according to AHo, and the antibody or antigen-binding fragment further comprises one or more framework sequence from MPK176-v1.3 and/or one or more framework sequence that is a variant of a MPK176-v1.3 framework sequence, wherein the one or more framework sequence of MPK176-v1.3 is according to AHo.

In certain embodiments, an antibody or antigen-binding fragment comprises a VH comprising a FRI, a FR2, a FR3, and/or a FR4 (or a variant of the FR1, FR2, FR3, and/or FR4 comprising one, two, three, four, or five amino acid substitutions (optionally comprising or consisting of one or more conservative substitution), insertions, and/or deletions) of the VH amino acid sequence set forth in SEQ ID NO.: 233 and a VL comprising a FR1, a FR2, a FR3, and/or a FR4 (or a variant of the FRI, FR2, FR3, and/or FR4 comprising one, two, three, four, or five amino acid substitutions (optionally comprising or consisting of one or more conservative substitution), insertions, and/or deletions) of the VL amino acid sequence set forth in SEQ ID NO.: 858. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment comprises a VH comprising a FRI, a FR2, a FR3, and/or a FR4 having at least 80%, at least 85%, at least 90%, at least 91%, at least 92%, at least 93%, at least 94%, at least 95%, at least 96%, at least 97%, at least 98%, or at least 99% itentity to, or comprising, consisting essentially of, or consisting of the respective FR1, FR2, FR3, or FR4 of the VH amino acid sequence set forth in SEQ ID NO.: 233 and a VL comprising a FRI, a FR2, a FR3, and/or a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FRI, FR2, FR3, or FR4 of the of the VL amino acid sequence set forth in SEQ ID NO.: 858. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment comprises a VH comprising a FRI, a FR2, a FR3, and a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FR1, FR2, FR3, or FR4 of the VH amino acid sequence set forth in SEQ ID NO.: 233 and a VL comprising a FRI, a FR2, a FR3, and a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FR1, FR2, FR3, or FR4 of SEQ ID NO.: 858. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment of the present disclosure that binds RSV-F and/or neutralizes RSV and may comprise one or more VH that binds RSV-F and may further comprise CDRs of a VH sequence according to SEQ ID NO.: 233 and may comprise one or more VL that binds RSV-F and may further comprise CDRs of a VL sequence according to SEQ ID NO.: 858, in which CDRs are as determined using any known CDR numbering method, including the Kabat, Chothia, EU, Martin (Enhanced Chothia), Contact, AbM, CCG, EU, or AHo, numbering method or a combination of two or more of theseIMGT, Martin (Enhanced Chothia), Contact, North, and AHo numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software. In certain embodiments, CDRs are according to the Kabat numbering method. In certain embodiments, CDRs are according to the AHo numbering method. In certain embodiments, CDRs are according to the North numbering method.

An antibody or antigen-binding fragment of the present disclosure that binds RSV-F and/or neutralizes RSV may comprise a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are determined according to the IMGT numbering system, and wherein: (i) optionally, the CDRH1 comprises or consists of the amino acid sequence set forth in SEQ ID NO.: 234, or a functional variant thereof comprising one, two, or three acid substitutions, one or more of which substitutions is optionally a conservative substitution and/or is a substitution to a germline-encoded amino acid; (ii) optionally, the CDRH2 comprises or consists of the amino acid sequence set forth in SEQ ID NO.: 235, or a functional variant thereof comprising one, two, or three amino acid substitutions, one or more of which substitutions is optionally a conservative substitution and/or is a substitution to a germline-encoded amino acid; (iii) optionally, the CDRH3 comprises or consists of the amino acid sequence set forth in any SEQ ID NO.: 236, or a functional variant thereof comprising one, two, or three amino acid substitutions, one or more of which substitutions is optionally a conservative substitution and/or is a substitution to a germline-encoded amino acid; (iv) optionally, the CDRL1 comprises or consists of the amino acid sequence set forth in SEQ ID NO.: 239, or a functional variant thereof comprising one, two, or three amino acid substitutions, one or more of which substitutions is optionally a conservative substitution and/or is a substitution to a germline-encoded amino acid; (v) optionally, the CDRL2 comprises or consists of the amino acid sequence set forth in SEQ ID NO.: 240, or a functional variant thereof comprising one, two, or three amino acid substitutions, one or more of which substitutions is optionally a conservative substitution and/or is a substitution to a germline-encoded amino acid; and/or (vi) optionally, the CDRL3 comprises or consists of the amino acid sequence set forth in SEQ ID NO.: 241, or a functional variant thereof comprising having one, two, or three amino acid substitutions, one or more of which substitutions is optionally a conservative substitution and/or is a substitution to a germline-encoded amino acid.

In specific embodiments, the antibody or antigen-binding fragment comprises i) a VH and a VL; or a CDRH1, CDRH2, CDRH3, CDRL1, CDRL2, and CDRL3, either as set forth in this “MPK176-v1.3 Antibodies” section or otherwise for a MPK176-v1.3 antibody or as determined by any CDR determination scheme disclosed herein, and ii) a Fc moiety. In certain embodiments, such an antibody or antigen-binding fragment comprises a CH and/or CL (including, potentially a kappa light chain constant region) or a fragment thereof associated with the VH or VL, respectively.

In specific embodiments, the antibody or antigen-binding fragment comprises a VH and a VL; or a CDRH1, CDRH2, CDRH3, CDRL1, CDRL2, and CDRL3, either as set forth in this “MPK176-v1.3Antibodies” section or otherwise for a MPK176-v1.3 antibody or as determined by any CDR determination scheme disclosed herein, and (A) a Fc moiety that comprises the substitution mutations: (i) G236A, L328V, and Q295E; (ii) G236A, P230A, and Q295E; (iii) G236A, R292P, and 1377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E; (xix) M428L/N434S; (xx) M428L/N434A; (xxi) G236A/A330L/I332E/M428L/N434S; (xxii) G236A/A330L/I332E/M428L/N434A; or (xxiii) any two or more of (i)-(xxii); or (B) an Fc moiety that comprises or consists of a Fc polypeptide or fragment thereof that comprises or consists of amino acid sequences having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs.: 664-700, optionally other than naturally occurring variants thereof, or that comprises or consists of, the amino acid sequences set forth in any one of SEQ ID NOs.: 672-700.

In certain embodiments, the antibody or antigen-binding fragment comprises i) a VH and a VL; or a CDRH1, CDRH2, CDRH3, CDRL1, CDRL2, and CDRL3, either as set forth in this “MPK176-v1.3 Antibodies” section or otherwise for a MPK176-v1.3 antibody or as determined by any CDR determination scheme disclosed herein and ii) an Fc moiety that comprises or consists of a Fc polypeptide or fragment thereof that comprises or consists of amino acid sequences having a sequence according to any one of SEQ ID NOs.: 679-684 and 688-690, or at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequence according to any one of SEQ ID NOs.: 679-684 and 688-690.

In certain embodiments, the antibody or antigen-binding fragment comprises a MPK176-v1.3-rIG1m3-LS antibody.

In certain embodiments, the antibody or antigen-binding fragment comprises a MPK176-v1.3-rIG1m17,1-LS antibody.

MPK176-v4.3 Antibodies In some embodiments, an antibody or antigen-binding fragment of the present disclosure is a MPK176-v4.3 antibody or antigen-binding fragment therof. Such an antibody or antigen-binding fragment thereof may bind RSV and/or neutralize RSV.

In some embodiments, a MPK176-v4.3 antibody may have a VH, a VL, CDRH1-H3, and/or CDRL1-L3 comprising or consisting of amino acid sequences or encoded by polynucleotides having sequences as follows:

| | SEQ ID NOs. | | | |
|---|---|---|---|---|
| Ab | CDRH1-3 (aa) | VH (aa/nt) | CDRL1-3 (aa) | VL (aa/nt) |
| MPK176-v4.3 | 234, 838, 236 | 837/836 | 239, 240, 241 | 858/857 |

In some embodiments, the VH and VL for a MPK176-v4.3 antibody or antigen-binding fragment that binds RSV-F and/or neutralizes RSV comprise or consist of a VH and VL having the sequences of SEQ ID NOs.: 837 and 858, respectively.

In certain embodiments, an antibody or antigen-binding fragment of the present disclosure binds RSV-F and/or neutralizes RSV and:
(i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in SEQ ID NO.: 837, wherein sequence variation is optionally limited to one or more framework regions and/or sequence variation comprises one or more substitution to a germline-encoded amino acid; and/or
(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in SEQ ID NO.: 858, wherein sequence variation is optionally limited to one or more framework regions and/or sequence variation comprises one or more substitution to a germline-encoded amino acid.

In some embodiments, variation as compared to SEQ ID NO.: 837 or SEQ ID NO.: 858 is limited to one or more framework region. In some embodiments, the variation comprises or consists of one, two, three, four, five, six, seven, eight, nine, or ten amino acid substitutions (any or all of which may be a conservative substitution), insertions, or deletions in the VH, in the VL, or in both. In some embodiments, the variation comprises one or more amino acid substitution, insertion, and/or deletion of an amino acid that is two, three, four, five, six, seven, eight, nine, ten, eleven, twelve, thirteen, fourteen, fifteen, or more amino acid positions away from an N-terminal and/or a C-terminal amino acid of a CDR.

Framework regions can be identified according to a numbering scheme (e.g., IMGT, Kabat, Chothia, North, EU, Martin (Enhanced Chothia), Contact, AbM, CCG, EU, or AHo, or a combination of two or more of these). The CDRs may be identified within a variable domain or within a heavy or light chain according to a numbering scheme or a combination of numbering schemes, and, preferably, the FRs may be identified using the same numbering scheme or combination of numbering schemes.

For example, in some embodiments, an antibody or antigen-binding fragment comprises CDRH1, CDRH2, CDRH3, CDRL1, CDRL2, and CDRL3 of MPK176-v4.3, wherein the CDRH1, CDRH2, CDRH3, CDRL1, CDRL2, and CDRL3 are according to IMGT (optionally IMGT-junction for CDRH3 and CDRL3), and the antibody or antigen-binding fragment further comprises one or more framework sequence from MPK176-v4.3 and/or one or more framework sequence that is a variant of a MPK176-v4.3framework sequence, wherein the one or more framework sequence of MPK176-v4.3 is according to IMGT (and if IMGT-junction is used for CDRH3 and CDRL3, accounting for this).

In other embodiments, an antibody or antigen-binding fragment comprises CDRH1, CDRH2, CDRH3, CDRL1, CDRL2, and CDRL3 of MPK176-v4.3, wherein the CDRH1, CDRH2, CDRH3, CDRL1, CDRL2, and CDRL3 are according to Kabat, and the antibody or antigen-binding fragment further comprises one or more framework sequence from MPK176-v4.3 and/or one or more framework sequence that is a variant of a MPK176-v4.3 framework sequence, wherein the one or more framework sequence of MPK176-v4.3 is according to Kabat.

In other embodiments, an antibody or antigen-binding fragment comprises CDRH1, CDRH2, CDRH3, CDRL1, CDRL2, and CDRL3 of MPK176-v4.3, wherein the CDRH1, CDRH2, CDRH3, CDRL1, CDRL2, and CDRL3 are according to Chothia, and the antibody or antigen-binding fragment further comprises one or more framework sequence from MPK176-v4.3 and/or one or more framework sequence that is a variant of a MPK176-v4.3 framework sequence, wherein the one or more framework sequence of MPK176-v4.3 is according to Chothia.

In other embodiments, an antibody or antigen-binding fragment comprises CDRH1, CDRH2, CDRH3, CDRL1, CDRL2, and CDRL3 of MPK176-v4.3, wherein the CDRH1, CDRH2, CDRH3, CDRL1, CDRL2, and CDRL3 are according to Martin (Enhanced Chothia), and the antibody or antigen-binding fragment further comprises one or more framework sequence from MPK176-v4.3 and/or one or more framework sequence that is a variant of a MPK176-v4.3 framework sequence, wherein the one or more framework sequence of MPK176-v4.3 is according to Martin (Enhanced Chothia).

In other embodiments, an antibody or antigen-binding fragment comprises CDRH1, CDRH2, CDRH3, CDRL1, CDRL2, and CDRL3 of MPK176-v4.3, wherein the CDRH1, CDRH2, CDRH3, CDRL1, CDRL2, and CDRL3 are according to AbM, and the antibody or antigen-binding fragment further comprises one or more framework sequence from MPK176-v4.3 and/or one or more framework sequence that is a variant of a MPK176-v4.3 framework sequence, wherein the one or more framework sequence of MPK176-v4.3 is according to AbM.

In other embodiments, an antibody or antigen-binding fragment comprises CDRH1, CDRH2, CDRH3, CDRL1, CDRL2, and CDRL3 of MPK176-v4.3, wherein the CDRH1, CDRH2, CDRH3, CDRL1, CDRL2, and CDRL3 are according to North, and the antibody or antigen-binding fragment further comprises one or more framework sequence from MPK176-v4.3 and/or one or more framework sequence that is a variant of a MPK176-v4.3 framework sequence, wherein the one or more framework sequence of MPK176-v4.3 is according to North.

In other embodiments, an antibody or antigen-binding fragment comprises CDRH1, CDRH2, CDRH3, CDRL1, CDRL2, and CDRL3 of, wherein the CDRH1, CDRH2, CDRH3, CDRL1, CDRL2, and CDRL3 are according to Contact, and the antibody or antigen-binding fragment further comprises one or more framework sequence from MPK176-v4.3 and/or one or more framework sequence that is a variant of a MPK176-v4.3 framework sequence, wherein the one or more framework sequence of MPK176-v4.3 is according to Contact.

In other embodiments, an antibody or antigen-binding fragment comprises CDRH1, CDRH2, CDRH3, CDRL1, CDRL2, and CDRL3 of MPK176-v4.3, wherein the CDRH1, CDRH2, CDRH3, CDRL1, CDRL2, and CDRL3 are according to CCG, and the antibody or antigen-binding fragment further comprises one or more framework sequence from MPK176-v4.3 and/or one or more framework sequence that is a variant of a MPK176-v4.3 framework sequence, wherein the one or more framework sequence of MPK176-v4.3 is according to CCG.

In other embodiments, an antibody or antigen-binding fragment comprises CDRH1, CDRH2, CDRH3, CDRL1, CDRL2, and CDRL3 of MPK176-v4.3, wherein the CDRH1, CDRH2, CDRH3, CDRL1, CDRL2, and CDRL3 are according to EU, and the antibody or antigen-binding fragment further comprises one or more framework sequence from MPK176-v4.3 and/or one or more framework sequence that is a variant of a MPK176-v4.3 framework sequence, wherein the one or more framework sequence of MPK176-v4.3 is according to EU.

In other embodiments, an antibody or antigen-binding fragment comprises CDRH1, CDRH2, CDRH3, CDRL1, CDRL2, and CDRL3 of MPK176-v4.3, wherein the CDRH1, CDRH2, CDRH3, CDRL1, CDRL2, and CDRL3 are according to AHo, and the antibody or antigen-binding fragment further comprises one or more framework sequence from MPK176-v4.3 and/or one or more framework sequence that is a variant of a MPK176-v4.3 framework sequence, wherein the one or more framework sequence of MPK176-v4.3 is according to AHo.

In certain embodiments, an antibody or antigen-binding fragment comprises a VH comprising a FRI, a FR2, a FR3, and/or a FR4 (or a variant of the FR1, FR2, FR3, and/or FR4 comprising one, two, three, four, or five amino acid substitutions (optionally comprising or consisting of one or more conservative substitution), insertions, and/or deletions) of the VH amino acid sequence set forth in SEQ ID NO.: 837 and a VL comprising a FR1, a FR2, a FR3, and/or a FR4 (or a variant of the FRI, FR2, FR3, and/or FR4 comprising one, two, three, four, or five amino acid substitutions (optionally comprising or consisting of one or more conservative substitution), insertions, and/or deletions) of the VL amino acid sequence set forth in SEQ ID NO.: 858. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment comprises a VH comprising a FRI, a FR2, a FR3, and/or a FR4 having at least 80%, at least 85%, at least 90%, at least 91%, at least 92%, at least 93%, at least 94%, at least 95%, at least 96%, at least 97%, at least 98%, or at least 99% itentity to, or comprising, consisting essentially of, or consisting of the respective FR1, FR2, FR3, or FR4 of the VH amino acid sequence set forth in SEQ ID NO.: 837 and a VL comprising a FRI, a FR2, a FR3, and/or a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FRI, FR2, FR3, or FR4 of the of the VL amino acid sequence set forth in SEQ ID NO.: 858. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment comprises a VH comprising a FRI, a FR2, a FR3, and a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FR1, FR2, FR3, or FR4 of the VH amino acid sequence set forth in SEQ ID NO.: 837 and a VL comprising a FRI, a FR2, a FR3, and a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FR1, FR2, FR3, or FR4 of SEQ ID NO.: 858. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment of the present disclosure that binds RSV-F and/or neutralizes RSV and may comprise one or more VH that binds RSV-F and may further comprise CDRs of a VH sequence according to SEQ ID NO.: 837 and may comprise one or more VL that binds RSV-F and may further comprise CDRs of a VL sequence according to SEQ ID NO.: 858, in which CDRs are as determined using any known CDR numbering method, including the Kabat, Chothia, EU, Martin (Enhanced Chothia), Contact, AbM, CCG, EU, or AHo, numbering method or a combination of two or more of theseIMGT, Martin (Enhanced Chothia), Contact, North, and AHo numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software. In certain embodiments, CDRs are according to the Kabat numbering method. In certain embodiments, CDRs are according to the AHo numbering method. In certain embodiments, CDRs are according to the North numbering method.

An antibody or antigen-binding fragment of the present disclosure that binds RSV-F and/or neutralizes RSV may comprise a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are determined according to the IMGT numbering system, and wherein: (i) optionally, the CDRH1 comprises or consists of the amino acid sequence set forth in SEQ ID NO.: 234, or a functional variant thereof comprising one, two, or three acid substitutions, one or more of which substitutions is optionally a conservative substitution and/or is a substitution to a germline-encoded amino acid; (ii) optionally, the CDRH2 comprises or consists of the amino acid sequence set forth in SEQ ID NO.: 838, or a functional variant thereof comprising one, two, or three amino acid substitutions, one or more of which substitutions is optionally a conservative substitution and/or is a substitution to a germline-encoded amino acid; (iii) optionally, the CDRH3 comprises or consists of the amino acid sequence set forth in any SEQ ID NO.: 236, or a functional variant thereof comprising one, two, or three amino acid substitutions, one or more of which substitutions is optionally a conservative substitution and/or is a substitution to a germline-encoded amino acid; (iv) optionally, the CDRL1 comprises or consists of the amino acid sequence set forth in SEQ ID NO.: 239, or a functional variant thereof comprising one, two, or three amino acid substitutions, one or more of which substitutions is optionally a conservative substitution and/or is a substitution to a germline-encoded amino acid; (v) optionally, the CDRL2 comprises or consists of the amino acid sequence set forth in SEQ ID NO.: 240, or a functional variant thereof comprising one, two, or three amino acid substitutions, one or more of which substitutions is optionally a conservative substitution and/or is a substitution to a germline-encoded amino acid; and/or (vi) optionally, the CDRL3 comprises or consists of the amino acid sequence set forth in SEQ ID NO.: 241, or a functional variant thereof comprising having one, two, or three amino acid substitutions, one or more of which substitutions is optionally a conservative substitution and/or is a substitution to a germline-encoded amino acid.

In specific embodiments, the antibody or antigen-binding fragment comprises i) a VH and a VL; or a CDRH1, CDRH2, CDRH3, CDRL1, CDRL2, and CDRL3, either as set forth in this “MPK176-v4.3 Antibodies” section or otherwise for a MPK176-v4.3 antibody or as determined by any CDR determination scheme disclosed herein, and ii) a Fc moiety. In certain embodiments, such an antibody or antigen-binding fragment comprises a CH and/or CL (including, potentially a kappa light chain constant region) or a fragment thereof associated with the VH or VL, respectively.

In specific embodiments, the antibody or antigen-binding fragment comprises a VH and a VL; or a CDRH1, CDRH2, CDRH3, CDRL1, CDRL2, and CDRL3, either as set forth in this “MPK176-v4.3Antibodies” section or otherwise for a MPK176-v4.3 antibody or as determined by any CDR determination scheme disclosed herein, and (A) a Fc moiety that comprises the substitution mutations: (i) G236A, L328V, and Q295E; (ii) G236A, P230A, and Q295E; (iii) G236A, R292P, and 1377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E; (xix) M428L/N434S; (xx) M428L/N434A; (xxi) G236A/A330L/I332E/M428L/N434S; (xxii) G236A/A330L/I332E/M428L/N434A; or (xxiii) any two or more of (i)-(xxii); or (B) an Fc moiety that comprises or consists of a Fc polypeptide or fragment thereof that comprises or consists of amino acid sequences having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs.: 664-700, optionally other than naturally occurring variants thereof, or that comprises or consists of, the amino acid sequences set forth in any one of SEQ ID NOs.: 672-700.

In certain embodiments, the antibody or antigen-binding fragment comprises i) a VH and a VL; or a CDRH1, CDRH2, CDRH3, CDRL1, CDRL2, and CDRL3, either as set forth in this “MPK176-v4.3 Antibodies” section or otherwise for a MPK176-v4.3 antibody or as determined by any CDR determination scheme disclosed herein and ii) an Fc moiety that comprises or consists of a Fc polypeptide or fragment thereof that comprises or consists of amino acid sequences having a sequence according to any one of SEQ ID NOs.: 679-684 and 688-690, or at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequence according to any one of SEQ ID NOs.: 679-684 and 688-690.

In certain embodiments, the antibody or antigen-binding fragment comprises a MPK176-v4.3-rIG1m3-LS antibody.

In certain embodiments, the antibody or antigen-binding fragment comprises a MPK176-v4.3-rIG1m17,1-LS antibody.

MPK201-v1.2 Antibodies In some embodiments, an antibody or antigen-binding fragment of the present disclosure is a MPK201-v1.2 antibody or antigen-binding fragment therof. Such an antibody or antigen-binding fragment thereof may bind RSV and/or neutralize RSV.

In some embodiments, a MPK201-v1.2 antibody may have a VH, a VL, CDRH1-H3, and/or CDRL1-L3 comprising or consisting of amino acid sequences or encoded by polynucleotides having sequences as follows:

| | SEQ ID NOs. | | | |
|---|---|---|---|---|
| Ab | CDRH1-3 (aa) | VH (aa/nt) | CDRL1-3 (aa) | VL (aa/nt) |
| MPK201-v1.2 | 137, 226, 358 | 357/891 | 142, 143, 848 | 847/846 |

In some embodiments, the VH and VL for a MPK201-v1.2 antibody or antigen-binding fragment that binds RSV-F and/or neutralizes RSV comprise or consist of a VH and VL having the sequences of SEQ ID NOs.: 357 and 847, respectively.

In certain embodiments, an antibody or antigen-binding fragment of the present disclosure binds RSV-F and/or neutralizes RSV and:
(i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in SEQ ID NO.: 357, wherein sequence variation is optionally limited to one or more framework regions and/or sequence variation comprises one or more substitution to a germline-encoded amino acid; and/or
(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in SEQ ID NO.: 847, wherein sequence variation is optionally limited to one or more framework regions and/or sequence variation comprises one or more substitution to a germline-encoded amino acid.

In some embodiments, variation as compared to SEQ ID NO.: 357 or SEQ ID NO.: 847 is limited to one or more framework region. In some embodiments, the variation comprises or consists of one, two, three, four, five, six, seven, eight, nine, or ten amino acid substitutions (any or all of which may be a conservative substitution), insertions, or deletions in the VH, in the VL, or in both. In some embodiments, the variation comprises one or more amino acid substitution, insertion, and/or deletion of an amino acid that is two, three, four, five, six, seven, eight, nine, ten, eleven, twelve, thirteen, fourteen, fifteen, or more amino acid positions away from an N-terminal and/or a C-terminal amino acid of a CDR.

Framework regions can be identified according to a numbering scheme (e.g., IMGT, Kabat, Chothia, North, EU, Martin (Enhanced Chothia), Contact, AbM, CCG, EU, or AHo, or a combination of two or more of these). The CDRs may be identified within a variable domain or within a heavy or light chain according to a numbering scheme or a combination of numbering schemes, and, preferably, the FRs may be identified using the same numbering scheme or combination of numbering schemes.

For example, in some embodiments, an antibody or antigen-binding fragment comprises CDRH1, CDRH2, CDRH3, CDRL1, CDRL2, and CDRL3 of MPK201-v1.2, wherein the CDRH1, CDRH2, CDRH3, CDRL1, CDRL2, and CDRL3 are according to IMGT (optionally IMGT-junction for CDRH3 and CDRL3), and the antibody or antigen-binding fragment further comprises one or more framework sequence from MPK201-v1.2 and/or one or more framework sequence that is a variant of a MPK201-v1.2framework sequence, wherein the one or more framework sequence of MPK201-v1.2 is according to IMGT (and if IMGT-junction is used for CDRH3 and CDRL3, accounting for this).

In other embodiments, an antibody or antigen-binding fragment comprises CDRH1, CDRH2, CDRH3, CDRL1, CDRL2, and CDRL3 of MPK201-v1.2, wherein the CDRH1, CDRH2, CDRH3, CDRL1, CDRL2, and CDRL3 are according to Kabat, and the antibody or antigen-binding fragment further comprises one or more framework sequence from MPK201-v1.2 and/or one or more framework sequence that is a variant of a MPK201-v1.2 framework sequence, wherein the one or more framework sequence of MPK201-v1.2 is according to Kabat.

In other embodiments, an antibody or antigen-binding fragment comprises CDRH1, CDRH2, CDRH3, CDRL1, CDRL2, and CDRL3 of MPK201-v1.2, wherein the CDRH1, CDRH2, CDRH3, CDRL1, CDRL2, and CDRL3 are according to Chothia, and the antibody or antigen-binding fragment further comprises one or more framework sequence from MPK201-v1.2 and/or one or more framework sequence that is a variant of a MPK201-v1.2 framework sequence, wherein the one or more framework sequence of MPK201-v1.2 is according to Chothia.

In other embodiments, an antibody or antigen-binding fragment comprises CDRH1, CDRH2, CDRH3, CDRL1, CDRL2, and CDRL3 of MPK201-v1.2, wherein the CDRH1, CDRH2, CDRH3, CDRL1, CDRL2, and CDRL3 are according to Martin (Enhanced Chothia), and the antibody or antigen-binding fragment further comprises one or more framework sequence from MPK201-v1.2 and/or one or more framework sequence that is a variant of a MPK201-v1.2 framework sequence, wherein the one or more framework sequence of MPK201-v1.2 is according to Martin (Enhanced Chothia).

In other embodiments, an antibody or antigen-binding fragment comprises CDRH1, CDRH2, CDRH3, CDRL1, CDRL2, and CDRL3 of MPK201-v1.2, wherein the CDRH1, CDRH2, CDRH3, CDRL1, CDRL2, and CDRL3 are according to AbM, and the antibody or antigen-binding fragment further comprises one or more framework sequence from MPK201-v1.2 and/or one or more framework sequence that is a variant of a MPK201-v1.2 framework sequence, wherein the one or more framework sequence of MPK201-v1.2 is according to AbM.

In other embodiments, an antibody or antigen-binding fragment comprises CDRH1, CDRH2, CDRH3, CDRL1, CDRL2, and CDRL3 of MPK201-v1.2, wherein the CDRH1, CDRH2, CDRH3, CDRL1, CDRL2, and CDRL3 are according to North, and the antibody or antigen-binding fragment further comprises one or more framework sequence from MPK201-v1.2 and/or one or more framework sequence that is a variant of a MPK201-v1.2 framework sequence, wherein the one or more framework sequence of MPK201-v1.2 is according to North.

In other embodiments, an antibody or antigen-binding fragment comprises CDRH1, CDRH2, CDRH3, CDRL1, CDRL2, and CDRL3 of, wherein the CDRH1, CDRH2, CDRH3, CDRL1, CDRL2, and CDRL3 are according to Contact, and the antibody or antigen-binding fragment further comprises one or more framework sequence from MPK201-v1.2 and/or one or more framework sequence that is a variant of a MPK201-v1.2 framework sequence, wherein the one or more framework sequence of MPK201-v1.2 is according to Contact.

In other embodiments, an antibody or antigen-binding fragment comprises CDRH1, CDRH2, CDRH3, CDRL1, CDRL2, and CDRL3 of MPK201-v1.2, wherein the CDRH1, CDRH2, CDRH3, CDRL1, CDRL2, and CDRL3 are according to CCG, and the antibody or antigen-binding fragment further comprises one or more framework sequence from MPK201-v1.2 and/or one or more framework sequence that is a variant of a MPK201-v1.2 framework sequence, wherein the one or more framework sequence of MPK201-v1.2 is according to CCG.

In other embodiments, an antibody or antigen-binding fragment comprises CDRH1, CDRH2, CDRH3, CDRL1, CDRL2, and CDRL3 of MPK201-v1.2, wherein the CDRH1, CDRH2, CDRH3, CDRL1, CDRL2, and CDRL3 are according to EU, and the antibody or antigen-binding fragment further comprises one or more framework sequence from MPK201-v1.2 and/or one or more framework sequence that is a variant of a MPK201-v1.2 framework sequence, wherein the one or more framework sequence of MPK201-v1.2 is according to EU.

In other embodiments, an antibody or antigen-binding fragment comprises CDRH1, CDRH2, CDRH3, CDRL1, CDRL2, and CDRL3 of MPK201-v1.2, wherein the CDRH1, CDRH2, CDRH3, CDRL1, CDRL2, and CDRL3 are according to AHo, and the antibody or antigen-binding fragment further comprises one or more framework sequence from MPK201-v1.2 and/or one or more framework sequence that is a variant of a MPK201-v1.2 framework sequence, wherein the one or more framework sequence of MPK201-v1.2 is according to AHo.

In certain embodiments, an antibody or antigen-binding fragment comprises a VH comprising a FRI, a FR2, a FR3, and/or a FR4 (or a variant of the FR1, FR2, FR3, and/or FR4 comprising one, two, three, four, or five amino acid substitutions (optionally comprising or consisting of one or more conservative substitution), insertions, and/or deletions) of the VH amino acid sequence set forth in SEQ ID NO.: 357 and a VL comprising a FR1, a FR2, a FR3, and/or a FR4 (or a variant of the FRI, FR2, FR3, and/or FR4 comprising one, two, three, four, or five amino acid substitutions (optionally comprising or consisting of one or more conservative substitution), insertions, and/or deletions) of the VL amino acid sequence set forth in SEQ ID NO.: 847. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment comprises a VH comprising a FRI, a FR2, a FR3, and/or a FR4 having at least 80%, at least 85%, at least 90%, at least 91%, at least 92%, at least 93%, at least 94%, at least 95%, at least 96%, at least 97%, at least 98%, or at least 99% itentity to, or comprising, consisting essentially of, or consisting of the respective FR1, FR2, FR3, or FR4 of the VH amino acid sequence set forth in SEQ ID NO.: 357 and a VL comprising a FRI, a FR2, a FR3, and/or a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FRI, FR2, FR3, or FR4 of the of the VL amino acid sequence set forth in SEQ ID NO.: 847. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment comprises a VH comprising a FRI, a FR2, a FR3, and a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FR1, FR2, FR3, or FR4 of the VH amino acid sequence set forth in SEQ ID NO.: 357 and a VL comprising a FRI, a FR2, a FR3, and a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FR1, FR2, FR3, or FR4 of SEQ ID NO.: 847. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment of the present disclosure that binds RSV-F and/or neutralizes RSV and may comprise one or more VH that binds RSV-F and may further comprise CDRs of a VH sequence according to SEQ ID NO.:357 and may comprise one or more VL that binds RSV-F and may further comprise CDRs of a VL sequence according to SEQ ID NO.: 847, in which CDRs are as determined using any known CDR numbering method, including the Kabat, Chothia, EU, Martin (Enhanced Chothia), Contact, AbM, CCG, EU, or AHo, numbering method or a combination of two or more of theseIMGT, Martin (Enhanced Chothia), Contact, North, and AHo numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software. In certain embodiments, CDRs are according to the Kabat numbering method. In certain embodiments, CDRs are according to the AHo numbering method. In certain embodiments, CDRs are according to the North numbering method.

An antibody or antigen-binding fragment of the present disclosure that binds RSV-F and/or neutralizes RSV may comprise a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are determined according to the IMGT numbering system, and wherein: (i) optionally, the CDRH1 comprises or consists of the amino acid sequence set forth in SEQ ID NO.: 137, or a functional variant thereof comprising one, two, or three acid substitutions, one or more of which substitutions is optionally a conservative substitution and/or is a substitution to a germline-encoded amino acid; (ii) optionally, the CDRH2 comprises or consists of the amino acid sequence set forth in SEQ ID NO.: 226, or a functional variant thereof comprising one, two, or three amino acid substitutions, one or more of which substitutions is optionally a conservative substitution and/or is a substitution to a germline-encoded amino acid; (iii) optionally, the CDRH3 comprises or consists of the amino acid sequence set forth in any SEQ ID NO.: 358, or a functional variant thereof comprising one, two, or three amino acid substitutions, one or more of which substitutions is optionally a conservative substitution and/or is a substitution to a germline-encoded amino acid; (iv) optionally, the CDRL1 comprises or consists of the amino acid sequence set forth in SEQ ID NO.: 142, or a functional variant thereof comprising one, two, or three amino acid substitutions, one or more of which substitutions is optionally a conservative substitution and/or is a substitution to a germline-encoded amino acid; (v) optionally, the CDRL2 comprises or consists of the amino acid sequence set forth in SEQ ID NO.: 143, or a functional variant thereof comprising one, two, or three amino acid substitutions, one or more of which substitutions is optionally a conservative substitution and/or is a substitution to a germline-encoded amino acid; and/or (vi) optionally, the CDRL3 comprises or consists of the amino acid sequence set forth in SEQ ID NO.: 848, or a functional variant thereof comprising having one, two, or three amino acid substitutions, one or more of which substitutions is optionally a conservative substitution and/or is a substitution to a germline-encoded amino acid.

In specific embodiments, the antibody or antigen-binding fragment comprises i) a VH and a VL; or a CDRH1, CDRH2, CDRH3, CDRL1, CDRL2, and CDRL3, either as set forth in this “MPK201-v1.2 Antibodies” section or otherwise for a MPK201-v1.2 antibody or as determined by any CDR determination scheme disclosed herein, and ii) a Fc moiety. In certain embodiments, such an antibody or antigen-binding fragment comprises a CH and/or CL (including, potentially a kappa light chain constant region) or a fragment thereof associated with the VH or VL, respectively.

In specific embodiments, the antibody or antigen-binding fragment comprises a VH and a VL; or a CDRH1, CDRH2, CDRH3, CDRL1, CDRL2, and CDRL3, either as set forth in this “MPK201-v1.2 Antibodies” section or otherwise for a MPK201-v1.2 antibody or as determined by any CDR determination scheme disclosed herein, and (A) a Fc moiety that comprises the substitution mutations: (i) G236A, L328V, and Q295E; (ii) G236A, P230A, and Q295E; (iii) G236A, R292P, and 1377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E; (xix) M428L/N434S; (xx) M428L/N434A; (xxi) G236A/A330L/I332E/M428L/N434S; (xxii) G236A/A330L/I332E/M428L/N434A; or (xxiii) any two or more of (i)-(xxii); or (B) an Fc moiety that comprises or consists of a Fc polypeptide or fragment thereof that comprises or consists of amino acid sequences having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs.: 664-700, optionally other than naturally occurring variants thereof, or that comprises or consists of, the amino acid sequences set forth in any one of SEQ ID NOs.: 672-700.

In certain embodiments, the antibody or antigen-binding fragment comprises i) a VH and a VL; or a CDRH1, CDRH2, CDRH3, CDRL1, CDRL2, and CDRL3, either as set forth in this “MPK201-v1.2 Antibodies” section or otherwise for a MPK201-v1.2 antibody or as determined by any CDR determination scheme disclosed herein and ii) an Fc moiety that comprises or consists of a Fc polypeptide or fragment thereof that comprises or consists of amino acid sequences having a sequence according to any one of SEQ ID NOs.: 679-684 and 688-690, or at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequence according to any one of SEQ ID NOs.: 679-684 and 688-690.

In certain embodiments, the antibody or antigen-binding fragment comprises a MPK201-v1.2-rIG1m3-LS antibody.

In certain embodiments, the antibody or antigen-binding fragment comprises a MPK201-v1.2-rIG1m17,1-LS antibody.

MPK201-v4.1 Antibodies In some embodiments, an antibody or antigen-binding fragment of the present disclosure is a MPK201-v4.1 antibody or antigen-binding fragment therof. Such an antibody or antigen-binding fragment thereof may bind RSV and/or neutralize RSV.

In some embodiments, a MPK201-v4.1 antibody may have a VH, a VL, CDRH1-H3, and/or CDRL1-L3 comprising or consisting of amino acid sequences or encoded by polynucleotides having sequences as follows:

| | SEQ ID NOs. | | | |
|---|---|---|---|---|
| Ab | CDRH1-3 (aa) | VH (aa/nt) | CDRL1-3 (aa) | VL (aa/nt) |
| MPK201-v4.1 | 814, 226, 358 | 899/898 | 142, 143, 231 | 360/845 |

In some embodiments, the VH and VL for a MPK201-v4.1 antibody or antigen-binding fragment that binds RSV-F and/or neutralizes RSV comprise or consist of a VH and VL having the sequences of SEQ ID NOs.: 899 and 360, respectively.

In certain embodiments, an antibody or antigen-binding fragment of the present disclosure binds RSV-F and/or neutralizes RSV and:
(i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in SEQ ID NO.: 899, wherein sequence variation is optionally limited to one or more framework regions and/or sequence variation comprises one or more substitution to a germline-encoded amino acid; and/or
(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in SEQ ID NO.: 360, wherein sequence variation is optionally limited to one or more framework regions and/or sequence variation comprises one or more substitution to a germline-encoded amino acid.

In some embodiments, variation as compared to SEQ ID NO.: 899 or SEQ ID NO.: 360 is limited to one or more framework region. In some embodiments, the variation comprises or consists of one, two, three, four, five, six, seven, eight, nine, or ten amino acid substitutions (any or all of which may be a conservative substitution), insertions, or deletions in the VH, in the VL, or in both. In some embodiments, the variation comprises one or more amino acid substitution, insertion, and/or deletion of an amino acid that is two, three, four, five, six, seven, eight, nine, ten, eleven, twelve, thirteen, fourteen, fifteen, or more amino acid positions away from an N-terminal and/or a C-terminal amino acid of a CDR.

Framework regions can be identified according to a numbering scheme (e.g., IMGT, Kabat, Chothia, North, EU, Martin (Enhanced Chothia), Contact, AbM, CCG, EU, or AHo, or a combination of two or more of these). The CDRs may be identified within a variable domain or within a heavy or light chain according to a numbering scheme or a combination of numbering schemes, and, preferably, the FRs may be identified using the same numbering scheme or combination of numbering schemes.

For example, in some embodiments, an antibody or antigen-binding fragment comprises CDRH1, CDRH2, CDRH3, CDRL1, CDRL2, and CDRL3 of MPK201-v4.1, wherein the CDRH1, CDRH2, CDRH3, CDRL1, CDRL2, and CDRL3 are according to IMGT (optionally IMGT-junction for CDRH3 and CDRL3), and the antibody or antigen-binding fragment further comprises one or more framework sequence from MPK201-v4.1 and/or one or more framework sequence that is a variant of a MPK201-v4.1 framework sequence, wherein the one or more framework sequence of MPK201-v4.1 is according to IMGT (and if IMGT-junction is used for CDRH3 and CDRL3, accounting for this).

In other embodiments, an antibody or antigen-binding fragment comprises CDRH1, CDRH2, CDRH3, CDRL1, CDRL2, and CDRL3 of MPK201-v4.1, wherein the CDRH1, CDRH2, CDRH3, CDRL1, CDRL2, and CDRL3 are according to Kabat, and the antibody or antigen-binding fragment further comprises one or more framework sequence from MPK201-v4.1 and/or one or more framework sequence that is a variant of a MPK201-v4.1 framework sequence, wherein the one or more framework sequence of MPK201-v4.1 is according to Kabat.

In other embodiments, an antibody or antigen-binding fragment comprises CDRH1, CDRH2, CDRH3, CDRL1, CDRL2, and CDRL3 of MPK201-v4.1, wherein the CDRH1, CDRH2, CDRH3, CDRL1, CDRL2, and CDRL3 are according to Chothia, and the antibody or antigen-binding fragment further comprises one or more framework sequence from MPK201-v4.1 and/or one or more framework sequence that is a variant of a MPK201-v4.1 framework sequence, wherein the one or more framework sequence of MPK201-v4.1 is according to Chothia.

In other embodiments, an antibody or antigen-binding fragment comprises CDRH1, CDRH2, CDRH3, CDRL1, CDRL2, and CDRL3 of MPK201-v4.1, wherein the CDRH1, CDRH2, CDRH3, CDRL1, CDRL2, and CDRL3 are according to Martin (Enhanced Chothia), and the antibody or antigen-binding fragment further comprises one or more framework sequence from MPK201-v4.1 and/or one or more framework sequence that is a variant of a MPK201-v4.1 framework sequence, wherein the one or more framework sequence of MPK201-v4.1 is according to Martin (Enhanced Chothia).

In other embodiments, an antibody or antigen-binding fragment comprises CDRH1, CDRH2, CDRH3, CDRL1, CDRL2, and CDRL3 of MPK201-v4.1, wherein the CDRH1, CDRH2, CDRH3, CDRL1, CDRL2, and CDRL3 are according to AbM, and the antibody or antigen-binding fragment further comprises one or more framework sequence from MPK201-v4.1 and/or one or more framework sequence that is a variant of a MPK201-v4.1 framework sequence, wherein the one or more framework sequence of MPK201-v4.1 is according to AbM.

In other embodiments, an antibody or antigen-binding fragment comprises CDRH1, CDRH2, CDRH3, CDRL1, CDRL2, and CDRL3 of MPK201-v4.1, wherein the CDRH1, CDRH2, CDRH3, CDRL1, CDRL2, and CDRL3 are according to North, and the antibody or antigen-binding fragment further comprises one or more framework sequence from MPK201-v4.1 and/or one or more framework sequence that is a variant of a MPK201-v4.1 framework sequence, wherein the one or more framework sequence of MPK201-v4.1 is according to North.

In other embodiments, an antibody or antigen-binding fragment comprises CDRH1, CDRH2, CDRH3, CDRL1, CDRL2, and CDRL3 of, wherein the CDRH1, CDRH2, CDRH3, CDRL1, CDRL2, and CDRL3 are according to Contact, and the antibody or antigen-binding fragment further comprises one or more framework sequence from MPK201-v4.1 and/or one or more framework sequence that is a variant of a MPK201-v4.1 framework sequence, wherein the one or more framework sequence of MPK201-v4.1 is according to Contact.

In other embodiments, an antibody or antigen-binding fragment comprises CDRH1, CDRH2, CDRH3, CDRL1, CDRL2, and CDRL3 of MPK201-v4.1, wherein the CDRH1, CDRH2, CDRH3, CDRL1, CDRL2, and CDRL3 are according to CCG, and the antibody or antigen-binding fragment further comprises one or more framework sequence from MPK201-v4.1 and/or one or more framework sequence that is a variant of a MPK201-v4.1 framework sequence, wherein the one or more framework sequence of MPK201-v4.1 is according to CCG.

In other embodiments, an antibody or antigen-binding fragment comprises CDRH1, CDRH2, CDRH3, CDRL1, CDRL2, and CDRL3 of MPK201-v4.1, wherein the CDRH1, CDRH2, CDRH3, CDRL1, CDRL2, and CDRL3 are according to EU, and the antibody or antigen-binding fragment further comprises one or more framework sequence from MPK201-v4.1 and/or one or more framework sequence that is a variant of a MPK201-v4.1 framework sequence, wherein the one or more framework sequence of MPK201-v4.1 is according to EU.

In other embodiments, an antibody or antigen-binding fragment comprises CDRH1, CDRH2, CDRH3, CDRL1, CDRL2, and CDRL3 of MPK201-v4.1, wherein the CDRH1, CDRH2, CDRH3, CDRL1, CDRL2, and CDRL3 are according to AHo, and the antibody or antigen-binding fragment further comprises one or more framework sequence from MPK201-v4.1 and/or one or more framework sequence that is a variant of a MPK201-v4.1 framework sequence, wherein the one or more framework sequence of MPK201-v4.1 is according to AHo.

In certain embodiments, an antibody or antigen-binding fragment comprises a VH comprising a FR1, a FR2, a FR3, and/or a FR4 (or a variant of the FR1, FR2, FR3, and/or FR4 comprising one, two, three, four, or five amino acid substitutions (optionally comprising or consisting of one or more conservative substitution), insertions, and/or deletions) of the VH amino acid sequence set forth in SEQ ID NO.: 899 and a VL comprising a FR1, a FR2, a FR3, and/or a FR4 (or a variant of the FRI, FR2, FR3, and/or FR4 comprising one, two, three, four, or five amino acid substitutions (optionally comprising or consisting of one or more conservative substitution), insertions, and/or deletions) of the VL amino acid sequence set forth in SEQ ID NO.: 360. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment comprises a VH comprising a FRI, a FR2, a FR3, and/or a FR4 having at least 80%, at least 85%, at least 90%, at least 91%, at least 92%, at least 93%, at least 94%, at least 95%, at least 96%, at least 97%, at least 98%, or at least 99% itentity to, or comprising, consisting essentially of, or consisting of the respective FR1, FR2, FR3, or FR4 of the VH amino acid sequence set forth in SEQ ID NO.: 899 and a VL comprising a FRI, a FR2, a FR3, and/or a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FRI, FR2, FR3, or FR4 of the of the VL amino acid sequence set forth in SEQ ID NO.: 360. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment comprises a VH comprising a FRI, a FR2, a FR3, and a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FR1, FR2, FR3, or FR4 of the VH amino acid sequence set forth in SEQ ID NO.: 899 and a VL comprising a FRI, a FR2, a FR3, and a FR4 having at least 80%, at least 85%, at least 90%, at least 91%, at least 92%, at least 93%, at least 94%, at least 95%, at least 96%, at least 97%, at least 98%, or at least 99% identity to, or comprising, consisting essentially of, or consisting of the respective FR1, FR2, FR3, or FR4 of SEQ ID NO.: 360. In some embodiments, the FRs are defined in accordance with the IMGT, Kabat, Chothia, North, EU, Martin (Enhanced Chothia), Contact, AbM, CCG, or AHo numbering system, or in accordance with any combination thereof.

In certain embodiments, an antibody or antigen-binding fragment of the present disclosure that binds RSV-F and/or neutralizes RSV and may comprise one or more VH that binds RSV-F and may further comprise CDRs of a VH sequence according to SEQ ID NO.: 899 and may comprise one or more VL that binds RSV-F and may further comprise CDRs of a VL sequence according to SEQ ID NO.: 360, in which CDRs are as determined using any known CDR numbering method, including the Kabat, Chothia, EU, Martin (Enhanced Chothia), Contact, AbM, CCG, EU, or AHo, numbering method or a combination of two or more of theseIMGT, Martin (Enhanced Chothia), Contact, North, and AHo numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software. In certain embodiments, CDRs are according to the Kabat numbering method. In certain embodiments, CDRs are according to the AHo numbering method. In certain embodiments, CDRs are according to the North numbering method.

An antibody or antigen-binding fragment of the present disclosure that binds RSV-F and/or neutralizes RSV may comprise a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are determined according to the IMGT numbering system, and wherein: (i) optionally, the CDRH1 comprises or consists of the amino acid sequence set forth in SEQ ID NO.: 814, or a functional variant thereof comprising one, two, or three acid substitutions, one or more of which substitutions is optionally a conservative substitution and/or is a substitution to a germline-encoded amino acid; (ii) optionally, the CDRH2 comprises or consists of the amino acid sequence set forth in SEQ ID NO.: 226, or a functional variant thereof comprising one, two, or three amino acid substitutions, one or more of which substitutions is optionally a conservative substitution and/or is a substitution to a germline-encoded amino acid; (iii) optionally, the CDRH3 comprises or consists of the amino acid sequence set forth in any SEQ ID NO.: 358, or a functional variant thereof comprising one, two, or three amino acid substitutions, one or more of which substitutions is optionally a conservative substitution and/or is a substitution to a germline-encoded amino acid; (iv) optionally, the CDRL1 comprises or consists of the amino acid sequence set forth in SEQ ID NO.: 142, or a functional variant thereof comprising one, two, or three amino acid substitutions, one or more of which substitutions is optionally a conservative substitution and/or is a substitution to a germline-encoded amino acid; (v) optionally, the CDRL2 comprises or consists of the amino acid sequence set forth in SEQ ID NO.: 143, or a functional variant thereof comprising one, two, or three amino acid substitutions, one or more of which substitutions is optionally a conservative substitution and/or is a substitution to a germline-encoded amino acid; and/or (vi) optionally, the CDRL3 comprises or consists of the amino acid sequence set forth in SEQ ID NO.: 231, or a functional variant thereof comprising having one, two, or three amino acid substitutions, one or more of which substitutions is optionally a conservative substitution and/or is a substitution to a germline-encoded amino acid.

In specific embodiments, the antibody or antigen-binding fragment comprises i) a VH and a VL; or a CDRH1, CDRH2, CDRH3, CDRL1, CDRL2, and CDRL3, either as set forth in this “MPK201-v4.1 Antibodies” section or otherwise for a MPK201-v4.1 antibody or as determined by any CDR determination scheme disclosed herein, and ii) a Fc moiety. In certain embodiments, such an antibody or antigen-binding fragment comprises a CH and/or CL (including, potentially a kappa light chain constant region) or a fragment thereof associated with the VH or VL, respectively.

In specific embodiments, the antibody or antigen-binding fragment comprises a VH and a VL; or a CDRH1, CDRH2, CDRH3, CDRL1, CDRL2, and CDRL3, either as set forth in this “MPK201-v4.1 Antibodies” section or otherwise for a MPK201-v4.1 antibody or as determined by any CDR determination scheme disclosed herein, and (A) a Fc moiety that comprises the substitution mutations: (i) G236A, L328V, and Q295E; (ii) G236A, P230A, and Q295E; (iii) G236A, R292P, and 1377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E; (xix) M428L/N434S; (xx) M428L/N434A; (xxi) G236A/A330L/I332E/M428L/N434S; (xxii) G236A/A330L/I332E/M428L/N434A; or (xxiii) any two or more of (i)-(xxii); or (B) an Fc moiety that comprises or consists of a Fc polypeptide or fragment thereof that comprises or consists of amino acid sequences having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs.: 664-700, optionally other than naturally occurring variants thereof, or that comprises or consists of, the amino acid sequences set forth in any one of SEQ ID NOs.: 672-700.

In certain embodiments, the antibody or antigen-binding fragment comprises i) a VH and a VL; or a CDRH1, CDRH2, CDRH3, CDRL1, CDRL2, and CDRL3, either as set forth in this “MPK201-v4.1 Antibodies” section or otherwise for a MPK201-v4.1 antibody or as determined by any CDR determination scheme disclosed herein and ii) an Fc moiety that comprises or consists of a Fc polypeptide or fragment thereof that comprises or consists of amino acid sequences having a sequence according to any one of SEQ ID NOs.: 679-684 and 688-690, or at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequence according to any one of SEQ ID NOs.: 679-684 and 688-690.

In certain embodiments, the antibody or antigen-binding fragment comprises a MPK201-v4.1-rIG1m3-LS antibody.

In certain embodiments, the antibody or antigen-binding fragment comprises a MPK201-v4.1-rIG1m17,1-LS antibody.

Polynucleotides, Vectors, and Host cells In another aspect, the present disclosure provides isolated polynucleotides that encode any of the presently disclosed antibodies or an antigen-binding fragment, or a portion thereof (e.g., a CDR, a VH, a VL, a heavy chain, or a light chain). In certain embodiments, the polynucleotide is codon-optimized for expression in a host cell. Once a coding sequence is known or identified, codon optimization can be performed using known techniques and tools, e.g., using the GenScript® OptimiumGenerm tool, or the like). Codon-optimized sequences include sequences that are partially codon-optimized (i.e., one or more codon is optimized for expression in the host cell) and those that are fully codon-optimized.

It will also be appreciated that polynucleotides encoding antibodies and antigen-binding fragments of the present disclosure may possess different nucleotide sequences while still encoding a same antibody or antigen-binding fragment due to, for example, the degeneracy of the genetic code, splicing, and the like.

In any of the presently disclosed embodiments, the polynucleotide can comprise deoxyribonucleic acid (DNA) or ribonucleic acid (RNA). In some embodiments, the RNA comprises messenger RNA (mRNA).

In specific embodiments, polynucleotides encoding antibodies or antigen-binding fragments that bind RSV-F comprise nucleotides encoding a VH in a polynucleotide sequence of any one of SEQ ID NOs.: 1, 135, 145, 158, 168, 174, 180, 188, 195, 201, 209, 214, 232, 249, 253, 260, 270, 276, 283, 292, 299, 308, 315, 320, 326, 331, 341, 346, 351, 356, 361, 368, 726, 736, 745, 754, 764, 774, 783, 793, 803, 811, 812, 816, 819, 822, 825, 827, 829, 830, 833, 836, 839, 842, 882, 885, 888, 891, 892, 895, 898, 900, 902, 128, 11, 37, 45, 52, 58, 68, 119, 72, 78, 85, 94, 21, 110, 29, 377, 385, 394, 403, 411, 415, 421, 425, 430, 439, 447, 450, 457, 462, 469, 474, 479, 484, 490, 496, 503, 512, 523, 526, 701, 706, 711, 715 and/or nucleotides encoding a VL in a polynucleotide sequence of any one of SEQ ID NOs.: 6, 140, 149, 154, 163, 171, 177, 184, 191, 198, 204, 211, 219, 228, 237, 246, 251, 256, 265, 273, 281, 287, 295, 304, 311, 318, 323, 329, 336, 344, 348, 354, 359, 366, 373, 731, 741, 750, 759, 769, 779, 788, 798, 807, 845, 846, 849, 850, 853, 854, 857, 859, 861, 863, 864, 867, 869, 872, 874, 876, 878, 880, 132, 16, 41, 49, 55, 63, 70, 124, 75, 82, 90, 97, 26, 115, 34, 381, 390, 399, 407, 413, 418, 423, 428, 433, 435, 443, 448, 454, 460, 465, 472, 477, 482, 486, 494, 500, 508, 517, 521, 525, 529, 703, 708, 709, 713, 714, and 717 and/or two or more portions of any of the preceding sequences encoding a CDRH1, CDRH2, CDRH3, CDRL1, CDRL2, or CDRL1, and/or a functional variant thereof comprising nucleotides that encode the same protein or a protein with one, two, or three amino acid substitutions, one or more of which substitutions is optionally a conservative substitution and/or is a substitution to a germline-encoded amino acid, and/or a nucleotide sequence having at least 80% (e.g., 80%, 85%, 90%, 91%, 92%, 93%, 94%, 95%, 96%, 97%, 98%, 99%, or more) identity to the nucleotide sequence.

In specific embodiments, polynucleotides encoding antibodies or antigen-binding fragments that bind RSV-F comprise nucleotides encoding a VH in a polynucleotide sequence of any one of SEQ ID NOs.: 1, 135, 145, 158, 168, 174, 180, 188, 195, 201, 209, 214, 232, 249, 253, 260, 270, 276, 283, 292, 299, 308, 315, 320, 326, 331, 341, 346, 351, 356, 361, 368, 726, 736, 745, 754, 764, 774, 783, 793, 803, 811, 812, 816, 819, 822, 825, 827, 829, 830, 833, 836, 839, 842, 882, 885, 888, 891, 892, 895, 898, 900, and 902 and/or nucleotides encoding a VL in a polynucleotide sequence of any one of SEQ ID NOs.: 6, 140, 149, 154, 163, 171, 177, 184, 191, 198, 204, 211, 219, 228, 237, 246, 251, 256, 265, 273, 281, 287, 295, 304, 311, 318, 323, 329, 336, 344, 348, 354, 359, 366, 373, 731, 741, 750, 759, 769, 779, 788, 798, 807, 845, 846, 849, 850, 853, 854, 857, 859, 861, 863, 864, 867, 869, 872, 874, 876, 878, and 880 and/or two or more portions of any ofthe preceding sequences encoding a CDRH1, CDRH2, CDRH3, CDRL1, CDRL2, or CDRL1, and/or a functional variant thereof comprising nucleotides that encode the same protein or a protein with one, two, or three amino acid substitutions, one or more of which substitutions is optionally a conservative substitution and/or is a substitution to a germline-encoded amino acid, and/or a nucleotide sequence having at least 80% (e.g., 80%, 85%, 90%, 91%, 92%, 93%, 94%, 95%, 96%, 97%, 98%, 99%, or more) identity to the nucleotide sequence.

In specific embodiments, polynucleotides encoding antibodies or antigen-binding fragments that bind MPV-F comprise nucleotides encoding a VH in a polynucleotide sequence of any one of SEQ ID NO.: 100, 129, 12, 38, 46, 53, 59, 69, 120, 73, 79, 86, 95, 22, 111, 30, 378, 386, 395, 404,412, 416, 422, 426, 431, 440, 451,458, 463, 470, 475, 480, 485, 491, 497, 532, 537, 539, 542, 545, 547, 550, 504, 513, 524, 527, 702, 707, 712, and 716, and/or nucleotides encoding a VL in a polynucleotide sequence of any one of SEQ ID NO.: 105, 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 553, 558, 560, 563, 567, 570, 572, 574, 704, and 710, and/or two or more portions of any of the preceding sequences encoding a CDRH1, CDRH2, CDRH3, CDRL1, CDRL2, or CDRL1, and/or a functional variant thereof comprising nucleotides that encode the same protein or a protein with one, two, or three amino acid substitutions, one or more of which substitutions is optionally a conservative substitution and/or is a substitution to a germline-encoded amino acid, and/or a nucleotide sequence having at least 80% (e.g., 80%, 85%, 90%, 91%, 92%, 93%, 94%, 95%, 96%, 97%, 98%, 99%, or more) identity to the nucleotide sequence.

In specific embodiments, polynucleotides encoding antibodies or antigen-binding fragments that bind MPV-F comprise nucleotides encoding a VH in a polynucleotide sequence of SEQ ID NO.: 100 and/or nucleotides encoding a VL in a polynucleotide sequence of SEQ ID NO.: 105, and/or two or more portions of any of the preceding sequences encoding a CDRH1, CDRH2, CDRH3, CDRL1, CDRL2, or CDRL1, and/or a functional variant thereof comprising nucleotides that encode the same protein or a protein with one, two, or three amino acid substitutions, one or more of which substitutions is optionally a conservative substitution and/or is a substitution to a germline-encoded amino acid, and/or a nucleotide sequence having at least 80% (e.g., 80%, 85%, 90%, 91%, 92%, 93%, 94%, 95%, 96%, 97%, 98%, 99%, or more) identity to the nucleotide sequence.

In specific embodiments, polynucleotides encoding antibodies or antigen-binding fragments that bind RSV-F and/or MPV-F comprise nucleotides encoding a VH in a polynucleotide sequence of any one of SEQ ID NOs.: 128, 11, 37, 45, 52, 58, 68, 119, 72, 78, 85, 94, 21, 110, 29, 377, 385, 394, 403, 411, 415, 421, 425, 430, 439, 447, 450, 457, 462, 469, 474, 479, 484, 490, 496, 503, 512, 523, 526, 701, 706, 711, and 715 and/or nucleotides encoding a VL in a polynucleotide sequence of any one of SEQ ID NOs.: 132, 16, 41, 49, 55, 63, 70, 124, 75, 82, 90, 97, 26, 115, 34, 381, 390, 399, 407, 413, 418, 423, 428, 433, 435, 443, 448, 454, 460, 465, 472, 477, 482, 486, 494, 500, 508, 517, 521, 525, 529, 703, 708, 709, 713, 714, and 717, and/or two or more portions of any of the preceding sequences encoding a CDRH1, CDRH2, CDRH3, CDRL1, CDRL2, or CDRL1, and/or a functional variant thereof comprising nucleotides that encode the same protein or a protein with one, two, or three amino acid substitutions, one or more of which substitutions is optionally a conservative substitution and/or is a substitution to a germline-encoded amino acid, and/or a nucleotide sequence having at least 80% (e.g., 80%, 85%, 90%, 91%, 92%, 93%, 94%, 95%, 96%, 97%, 98%, 99%, or more) identity to the nucleotide sequence.

In specific embodiments, polynucleotides encoding antibodies or antigen-binding fragments that bind RSV-F and/or MPV-F comprise nucleotides encoding a VH in a polynucleotide sequence of any one of SEQ ID NOs.: 531, 536, 538, 541, 544, 546, and 549 and/or nucleotides encoding a VL in a polynucleotide sequence of any one of SEQ ID NOs.: 552, 557, 576, 562, 566, 569, 571, and 573, and/or two or more portions of any of the preceding sequences encoding a CDRH1, CDRH2, CDRH3, CDRL1, CDRL2, or CDRL1, and/or a functional variant thereof comprising nucleotides that encode the same protein or a protein with one, two, or three amino acid substitutions, one or more of which substitutions is optionally a conservative substitution and/or is a substitution to a germline-encoded amino acid, and/or a nucleotide sequence having at least 80% (e.g., 80%, 85%, 90%, 91%, 92%, 93%, 94%, 95%, 96%, 97%, 98%, 99%, or more) identity to the nucleotide sequence.

MPK190-v1.3 Polynucleotides In specific embodiments, polynucleotides encoding antibodies or antigen-binding fragments that bind RSV-F and MPV-F comprise nucleotides encoding a VH in a polynucleotide sequence of SEQ ID NO.: 702 and/or nucleotides encoding a VL in a polynucleotide sequence of SEQ ID NO.: 704 and/or two or more portions of any of the preceding sequences encoding a CDRH1, CDRH2, CDRH3, CDRL1, CDRL2, or CDRL1, and/or a functional variant thereof comprising nucleotides that encode the same protein or a protein with one, two, or three amino acid substitutions, one or more of which substitutions is optionally a conservative substitution and/or is a substitution to a germline-encoded amino acid, and/or a nucleotide sequence having at least 80% (e.g., 80%, 85%, 90%, 91%, 92%, 93%, 94%, 95%, 96%, 97%, 98%, 99%, or more) identity to the nucleotide sequence.

MPK65-v2-v1.2 Polynucleotides In specific embodiments, polynucleotides encoding antibodies or antigen-binding fragments that bind RSV-F comprise nucleotides encoding a VH in a polynucleotide sequence of SEQ ID NO.: 135 and/or nucleotides encoding a VL in a polynucleotide sequence of SEQ ID NO.: 850 and/or two or more portions of any of the preceding sequences encoding a CDRH1, CDRH2, CDRH3, CDRL1, CDRL2, or CDRL1, and/or a functional variant thereof comprising nucleotides that encode the same protein or a protein with one, two, or three amino acid substitutions, one or more of which substitutions is optionally a conservative substitution and/or is a substitution to a germline-encoded amino acid, and/or a nucleotide sequence having at least 80% (e.g., 80%, 85%, 90%, 91%, 92%, 93%, 94%, 95%, 96%, 97%, 98%, 99%, or more) identity to the nucleotide sequence.

### MPK65-v2-v3.1 Polynucleotides

In specific embodiments, polynucleotides encoding antibodies or antigen-binding fragments that bind RSV-F comprise nucleotides encoding a VH in a polynucleotide sequence of SEQ ID NO.: 816 and/or nucleotides encoding a VL in a polynucleotide sequence of SEQ ID NO.: 140 and/or two or more portions of any of the preceding sequences encoding a CDRH1, CDRH2, CDRH3, CDRL1, CDRL2, or CDRL1, and/or a functional variant thereof comprising nucleotides that encode the same protein or a protein with one, two, or three amino acid substitutions, one or more of which substitutions is optionally a conservative substitution and/or is a substitution to a germline-encoded amino acid, and/or a nucleotide sequence having at least 80% (e.g., 80%, 85%, 90%, 91%, 92%, 93%, 94%, 95%, 96%, 97%, 98%, 99%, or more) identity to the nucleotide sequence.

### MPK176-v1.3 Polynucleotides

In specific embodiments, polynucleotides encoding antibodies or antigen-binding fragments that bind RSV-F comprise nucleotides encoding a VH in a polynucleotide sequence of SEQ ID NO.: 232 and/or nucleotides encoding a VL in a polynucleotide sequence of SEQ ID NO.: 857 and/or two or more portions of any of the preceding sequences encoding a CDRH1, CDRH2, CDRH3, CDRL1, CDRL2, or CDRL1, and/or a functional variant thereof comprising nucleotides that encode the same protein or a protein with one, two, or three amino acid substitutions, one or more of which substitutions is optionally a conservative substitution and/or is a substitution to a germline-encoded amino acid, and/or a nucleotide sequence having at least 80% (e.g., 80%, 85%, 90%, 91%, 92%, 93%, 94%, 95%, 96%, 97%, 98%, 99%, or more) identity to the nucleotide sequence.

MPK176-v4.3 Polynucleotides In specific embodiments, polynucleotides encoding antibodies or antigen-binding fragments that bind RSV-F comprise nucleotides encoding a VH in a polynucleotide sequence of SEQ ID NO.: 236 and/or nucleotides encoding a VL in a polynucleotide sequence of SEQ ID NO.: 857 and/or two or more portions of any of the preceding sequences encoding a CDRH1, CDRH2, CDRH3, CDRL1, CDRL2, or CDRL1, and/or a functional variant thereof comprising nucleotides that encode the same protein or a protein with one, two, or three amino acid substitutions, one or more of which substitutions is optionally a conservative substitution and/or is a substitution to a germline-encoded amino acid, and/or a nucleotide sequence having at least 80% (e.g., 80%, 85%, 90%, 91%, 92%, 93%, 94%, 95%, 96%, 97%, 98%, 99%, or more) identity to the nucleotide sequence.

MPK201-v1.2 Polynucleotides In specific embodiments, polynucleotides encoding antibodies or antigen-binding fragments that bind RSV-F comprise nucleotides encoding a VH in a polynucleotide sequence of SEQ ID NO.: 356 and/or nucleotides encoding a VL in a polynucleotide sequence of SEQ ID NO.: 846 and/or two or more portions of any of the preceding sequences encoding a CDRH1, CDRH2, CDRH3, CDRL1, CDRL2, or CDRL1, and/or a functional variant thereof comprising nucleotides that encode the same protein or a protein with one, two, or three amino acid substitutions, one or more of which substitutions is optionally a conservative substitution and/or is a substitution to a germline-encoded amino acid, and/or a nucleotide sequence having at least 80% (e.g., 80%, 85%, 90%, 91%, 92%, 93%, 94%, 95%, 96%, 97%, 98%, 99%, or more) identity to the nucleotide sequence.

MPK201-v4.1 Polynucleotides In specific embodiments, polynucleotides encoding antibodies or antigen-binding fragments that bind RSV-F comprise nucleotides encoding a VH in a polynucleotide sequence of SEQ ID NO.: 898 and/or nucleotides encoding a VL in a polynucleotide sequence of SEQ ID NO.: 359 and/or two or more portions of any of the preceding sequences encoding a CDRH1, CDRH2, CDRH3, CDRL1, CDRL2, or CDRL1, and/or a functional variant thereof comprising nucleotides that encode the same protein or a protein with one, two, or three amino acid substitutions, one or more of which substitutions is optionally a conservative substitution and/or is a substitution to a germline-encoded amino acid, and/or a nucleotide sequence having at least 80% (e.g., 80%, 85%, 90%, 91%, 92%, 93%, 94%, 95%, 96%, 97%, 98%, 99%, or more) identity to the nucleotide sequence.

Vectors are also provided, wherein the vectors comprise or contain a polynucleotide as disclosed herein (e.g., a polynucleotide that encodes an antibody or antigen-binding fragment that binds to RSV-F and/or MPV-F). A vector can comprise any one or more of the vectors disclosed herein. In particular embodiments, a vector is provided that comprises a DNA plasmid construct encoding the antibody or antigen-binding fragment, or a portion thereof (e.g., so-called “DMAb”; see, e.g., Muthumani et al., JInfectDis. 214(3):369-378 (2016); Muthumani et al., Hum Vaccin Immunother 9:2253-2262 (2013)); Flingai et al., Sci Rep. 5:12616 (2015); and Elliott et al., NPJ Vaccines 18 (2017), which antibody-coding DNA constructs and related methods of use, including administration of the same, are incorporated herein by reference). In certain embodiments, a DNA plasmid construct comprises a single open reading frame encoding a heavy chain and a light chain (or a VH and a VL) of the antibody or antigen-binding fragment, wherein the sequence encoding the heavy chain and the sequence encoding the light chain are optionally separated by polynucleotide encoding a protease cleavage site and/or by a polynucleotide encoding a self-cleaving peptide. In some embodiments, the substituent components of the antibody or antigen-binding fragment are encoded by a polynucleotide comprised in a single plasmid. In other embodiments, the substituent components of the antibody or antigen-binding fragment are encoded by a polynucleotide comprised in two or more plasmids (e.g., a first plasmid comprises a polynucleotide encoding a heavy chain, VH, or VH+CH, and a second plasmid comprises a polynucleotide encoding the cognate light chain, VL, or VL+CL). In certain embodiments, a single plasmid comprises a polynucleotide encoding a heavy chain and/or a light chain from two or more antibodies or antigen-binding fragments of the present disclosure. An exemplary expression vector is pVaxl, available from Invitrogen®. A DNA plasmid of the present disclosure can be delivered to a subject by, for example, electroporation (e.g., intramuscular electroporation), or with an appropriate formulation (e.g., hyaluronidase).

In a further aspect, the present disclosure also provides a host cell expressing an antibody or antigen-binding fragment according to the present disclosure; or comprising or containing a vector or polynucleotide according the present disclosure.

Examples of such cells include but are not limited to, eukaryotic cells, e.g., yeast cells, animal cells, insect cells, plant cells; and prokaryotic cells, including E. coli. In some embodiments, the cells are mammalian cells. In certain such embodiments, the cells are a mammalian cell line such as CHO cells (e.g., DHFR-CHO cells (Urlaub et al., PNAS 77:4216 (1980)), human embryonic kidney cells (e.g., HEK293T cells), PER.C6 cells, Y0 cells, Sp2/0 cells. NS0 cells, human liver cells, e.g. Hepa RG cells, myeloma cells or hybridoma cells.

Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biology, Vol. 248 (B. K. C. Lo, ed., Humana Press, Totowa, N.J.), pp. 255-268 (2003).

In certain embodiments, a host cell is a prokaryotic cell, such as an E. coli. The expression of peptides in prokaryotic cells such as E. coli is well established (see, e.g., Pluckthun, A. Bio Technology 9:545-551 (1991). For example, antibodies may be produced in bacteria, in particular when glycosylation and Fc effector function are not needed. For expression of antibody fragments and polypeptides in bacteria, see, e.g., U.S. Pat. Nos. 5,648,237; 5,789,199; and 5,840,523.

In particular embodiments, the cell may be transfected with a vector according to the present description with an expression vector. The term “transfection” refers to the introduction of nucleic acid molecules, such as DNA or RNA (e.g. mRNA) molecules, into cells, such as into eukaryotic cells. In the context of the present description, the term “transfection” encompasses any method known to the skilled person for introducing nucleic acid molecules into cells, such as into eukaryotic cells, including into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine, etc. In certain embodiments, the introduction is non-viral.

Moreover, host cells of the present disclosure may be transfected stably or transiently with a vector according to the present disclosure, e.g. for expressing an antibody, or an antigen-binding fragment, according to the present disclosure. In such embodiments, the cells may be stably transfected with the vector as described herein. Alternatively, cells may be transiently transfected with a vector according to the present disclosure encoding an antibody or antigen-binding fragment as disclosed herein. In any of the presently disclosed embodiments, a polynucleotide may be heterologous to the host cell.

Accordingly, the present disclosure also provides recombinant host cells that heterologously express an antibody or antigen-binding fragment of the present disclosure. For example, the cell may be of a species that is different to the species from which the antibody was fully or partially obtained (e.g., CHO cells expressing a human antibody or an engineered human antibody). In some embodiments, the cell type of the host cell does not express the antibody or antigen-binding fragment in nature. Moreover, the host cell may impart a post-translational modification (PTM; e.g., glycosylation or fucosylation), or a lack thereof, on the antibody or antigen-binding fragment that is not present in a native state of the antibody or antigen-binding fragment (or in a native state of a parent antibody from which the antibody or antigen binding fragment was engineered or derived). Such a PTM, or a lack thereof, may result in a functional difference (e.g., reduced immunogenicity). Accordingly, an antibody or antigen-binding fragment of the present disclosure that is produced by a host cell as disclosed herein may include one or more post-translational modification that is distinct from the antibody (or parent antibody) in its native state (e.g., a human antibody produced by a host cell can comprise one or more post-translational modification, or can include fewer post-translational modification(s), such that it is distinct from the antibody when isolated from the human and/or produced by the native human B cell or plasma cell).

Insect cells useful expressing a binding protein of the present disclosure are known in the art and include, for example, Spodopterafrugipera Sf9 cells, Trichoplusia ni BTI-TN5B1-4 cells, and Spodopterafrugipera Sf5WT01 “Mimic^{T}M” cells. See, e.g., Palmberger et al., J Biotechnol. 153(3-4):160-166 (2011). Numerous baculoviral strains have been identified which may be used in conjunction with insect cells, particularly for transfection of Spodoptera frugiperda cells.

Eukaryotic microbes such as filamentous fungi or yeast are also suitable hosts for cloning or expressing protein-encoding vectors, and include fungi and yeast strains with “humanized” glycosylation pathways, resulting in the production of an antibody with a partially or fully human glycosylation pattern. See Gemgross, Nat. Biotech. 22:1409-1414 (2004); Li et al., Nat. Biotech. 24:210⁻²¹⁵ (2006).

Plant cells can also be utilized as hosts for expressing an antibody or antigen-binding fragment of the present disclosure. For example, PLANTIBODIES™ technology (described in, for example, U.S. Pat. Nos. 5,959,177; 6,040,498; 6,420,548; 7,125,978; and 6,417,429) employs transgenic plants to produce antibodies.

In certain embodiments, the host cell comprises a mammalian cell. In particular embodiments, the host cell is a CHO cell, an ExpiCHO cell, a HEK293 cell, a PER.C6 cell, a YO cell, a Sp2/0 cell, a NS0 cell, a human liver cell, a myeloma cell, or a hybridoma cell.

In a related aspect, the present disclosure provides methods for producing an antibody or antigen-binding fragment, wherein the methods comprise culturing a host cell of the present disclosure under conditions and for a time sufficient to produce the antibody, or the antigen-binding fragment. Methods useful for isolating and purifying recombinantly produced antibodies, by way of example, may include obtaining supernatants from suitable host cell/vector systems that secrete the recombinant antibody into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment.

Methods for large scale production of one or more of the isolated/recombinant antibody described herein include batch cell culture, which is monitored and controlled to maintain appropriate culture conditions. Purification of soluble antibodies may be performed according to methods described herein and known in the art and that comport with laws and guidelines of domestic and foreign regulatory agencies.

Compositions Also provided herein are compositions that comprise a presently disclosed antibody, antigen-binding fragment, polynucleotide, vector, or host cell, singly or in any combination, and can further comprise a pharmaceutically acceptable carrier, excipient, or diluent. Such compositions, as well as carriers, excipients, and diluents, are discussed in further detail herein. In some embodiments, the composition includes two or more antibodies or antigen-binding fragments thereof, one of which is described in Table 2 or Table 20, has a VH and/or VL according to an antibody described in Table 2 or Table 20, or having at least 85%, at least 90%, at least 95%, or at least 98% sequence identity thereto, or having a combination of CDRH1-3 and/or CDRL1-3 (according to any numbering scheme, particularly IMGT) according to any antibody described in Table 2 or Table 20, and the second of which is described in Table 3 or Table 4, has a VH and/or VL according to an antibody described in Table 3 or Table 4, or having at least 85%, at least 90%, at least 95%, or at least 98% sequence identity thereto, or having a combination of CDRH1-3 and/or CDRL1-3 (according to any numbering scheme, particularly IMGT) according to any antibody described in Table 3 or Table 4.

In some embodiments including two antibodies or antigen-binding fragments thereof, one antibody may specifically bind both RSV and MPV, while the second antibody specifically binds only RSV or MPV. In other embodiments, one antibody may specifically bind only RSV while the second antibody specifically binds only MPV. In still other embodiments both antibodies may bind both RSV and MPV, only RSV, or only MPV.

In certain embodiments, including two antibodies or antigen-binding fragments, at least one antibody or antigen-binding fragment comprises at least a VH and VL or CDRs of the VH and VL of at least one of i) MPK65-v2-v1.2, MPK65-v2-v3.1, MPK176-v1.3, MPK76-v43., MPK201-v1.2, and MPK 201-v1.4; or ii) a VH and a VL set forth in any one of SEQ ID NOs.: 136 and 851; 814 and 141; 233 and 858; 837 and 858; 357 and 847; and 899 and 360, respectively.

In certain embodiments, including two antibodies or antigen-binding fragments, at least one antibody or antigen-binding fragment comprises at least a VH and VL or CDRs of the VH and VL of MPK190-v1.3 or a VH and VL as set forth in SEQ ID NOs.: 702 and 704.

In certain embodiments, including two antibodies or antigen-binding fragments, at least a first antibody or antigen-binding fragment comprises at least (A) a first VH and VL or first CDRs of the VH and VL of at least one of i) MPK65-v2-v1.2, MPK65-v2-v3.1, MPK176-v1.3, MPK76-v43., MPK201-v1.2, and MPK 201-v1.4; or ii) a VH and a VL set forth in any one of SEQ ID NOs.: 136 and 851; 814 and 141; 233 and 858; 837 and 858; 357 and 847; and 899 and 360, respectively; and (B) at least a second antibody or antigen-bindgin fragment comprises at least a second VH and VL or second CDRs of the VH and VL of MPK190-v1.3 or a VH and VL as set forth in SEQ ID NOs.: 702 and 704. In some embodiments, these first and second antibodies or antigen-binding fragments may be distinct antibodies, not fragments or components of a single, multispecific antibody.

In certain embodiments, a composition comprises a first vector comprising a first plasmid, and a second vector comprising a second plasmid, wherein the first plasmid comprises a polynucleotide encoding a heavy chain, VH, or VH+CH, and a second plasmid comprises a polynucleotide encoding the cognate light chain, VL, or VL+CL of the antibody or antigen-binding fragment. In certain embodiments, a composition comprises a polynucleotide (e.g., mRNA) coupled to a suitable delivery vehicle or carrier. Exemplary vehicles or carriers for administration to a human subject include a lipid or lipid-derived delivery vehicle, such as a liposome, solid lipid nanoparticle, oily suspension, submicron lipid emulsion, lipid microbubble, inverse lipid micelle, cochlear liposome, lipid microtubule, lipid microcylinder, or lipid nanoparticle (LNP) or a nanoscale platform (see, e.g., Li et al. Wilery Interdiscip Rev.

NanomedNanobiotechnol. 11(2):e1530 (2019)). Principles, reagents, and techniques for designing appropriate mRNA and formulating mRNA-LNP and delivering the same are described in, for example, Pardi et al. (J Control Release 217345-351 (2015)); Thess et al. (Mol Ther 23: 1456-1464 (2015)); Thran et al. (EMBO Mol Med 9(10):1434-1448 (2017); Kose et al. (Sci. Immunol. 4 eaaw6647 (2019); and Sabnis et al. (Mol. Ther 26:1509-1519 (2018)), which techniques, include capping, codon optimization, nucleoside modification, purification of mRNA, incorporation of the mRNA into stable lipid nanoparticles (e.g., ionizable cationic lipid/phosphatidylcholine/cholesterol/PEG-lipid; ionizable lipid:distearoyl PC:cholesterol: polyethylene glycol lipid), and subcutaneous, intramuscular, intradermal, intravenous, intraperitoneal, and intratracheal administration of the same, are incorporated herein by reference.

In some embodiments, the composition includes two or more antibodies or antigen-binding fragment thereof as described herein in Table 2 or Table 20, having a VH and/or a VL according to an antibody described in Table 2 or Table 20, or having at least 85%, at least 90%, at least 95%, or at least 98% sequence identity thereto, or having a combination of CDRH1-3 and/or CDRL1-3 according to any antibody described in Table 2 or Table 20.

Methods and Uses Also provided herein are methods for use of an antibody or antigen-binding fragment, nucleic acid, vector, cell, or composition of the present disclosure in the diagnosis of a RSV and/or MPV infection (e.g., in a human subject, or in a sample obtained from a human subject).

Methods of diagnosis (e.g., in vitro, ex vivo) may include contacting an antibody, antibody fragment (e.g., antigen binding fragment) with a sample. Such samples may be isolated from a subject, for Example 5n isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood. The methods of diagnosis may also include the detection of an antigen/antibody complex, in particular following the contacting of an antibody or antibody fragment with a sample. Such a detection step can be performed at the bench, i.e. without any contact to the human or animal body.

Examples of detection methods are well-known to the person skilled in the art and include, e.g., ELISA (enzyme-linked immunosorbent assay), including direct, indirect, and sandwich ELISA.

Also provided herein are methods of treating a subject using an antibody or antigen-binding fragment of the present disclosure, or a composition comprising the same, wherein the subject has, is believed to have, or is at risk for having an infection by a RSV and/or a MPV. “Treat,” “treatment,” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising an antibody or composition of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof. In certain embodiments, therapeutic or prophylactic/preventive benefit includes reduction or prevention of hospitalization for treatment of a RSV and/or MPV infection (i.e., in a statistically significant manner). In certain embodiments, therapeutic or prophylactic/preventive benefit includes a reduced duration of hospitalization for treatment of a RSV and/or a MPV infection (i.e., in a statistically significant manner). In certain embodiments, therapeutic or prophylactic/preventive benefit includes a reduced or abrogated need for respiratory intervention, such as intubation and/or the use of a respirator device. In certain embodiments, therapeutic or prophylactic/preventive benefit includes reversing a late-stage disease pathology and/or reducing mortality.

A “therapeutically effective amount” or “effective amount” of an antibody, antigen-binding fragment, polynucleotide, vector, host cell, or composition of this disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially, sequentially, or simultaneously.

Accordingly, in certain embodiments, methods are provided for treating a RSV and/or MPV infection in a subject, wherein the methods comprise administering to the subject an effective amount of an antibody, antigen-binding fragment, polynucleotide, vector, host cell, or composition as disclosed herein.

Subjects that can be treated by the present disclosure are, in general, human and other primate subjects, such as monkeys and apes for veterinary medicine purposes. Other model organisms, such as mice and rats, may also be treated according to the present disclosure. In any of the aforementioned embodiments, the subject may be a human subject. The subjects can be male or female and can be any suitable age, including infant, juvenile, adolescent, adult, and geriatric subjects.

A number of criteria are believed to contribute to high risk for severe symptoms or death associated with a RSV and/or MPV infection. These include, but are not limited to, age, occupation, general health, pre-existing health conditions, locale, and lifestyle habits. In some embodiments, a subject treated according to the present disclosure comprises one or more risk factors.

In certain embodiments, a human subject treated according to the present disclosure is an infant, a child, a young adult, an adult of middle age, or an elderly person. In certain embodiments, a human subject treated according to the present disclosure is less than 1 year old, or is 1 to 5 years old, or is between 5 and 125 years old (e.g., 5, 10, 15, 20, 25, 30, 35, 40, 45, 50, 55, 60, 65, 70, 75, 80, 85, 90, 95, 100, 105, 110, 115, or 125 years old, including any and all ages therein or therebetween). In certain embodiments, a human subject treated according to the present disclosure is 0-19 years old, 20-44 years old, 45-54 years old, 55-64 years old, 65-74 years old, 75-84 years old, or 85 years old, or older. Persons of middle, and especially of elderly age are believed to be at particular risk. In particular embodiments, the human subject is 45-54 years old, 55-64 years old, 65-74 years old, 75-84 years old, or 85 years old, or older. In some embodiments, the human subject is male. In some embodiments, the human subject is female.

In certain embodiments, a subject treated according to the present disclosure has received a vaccine for a RSV and/or MPV and the vaccine is determined to be ineffective, e.g., by post-vaccine infection or symptoms in the subject, by clinical diagnosis or scientific or regulatory consensus.

Prophylaxis of infection with RSV and/or MPV refers in particular to prophylactic settings, wherein the subject was not diagnosed with infection with RSV and/or MPV virus (either no diagnosis was performed or diagnosis results were negative) and/or the subject does not show or experience symptoms of infection with RSV and/or MPV. Prophylaxis of infection with RSV and/or MPV is particularly useful in subjects at greater risk of infection, severe disease, or complications when infected, babies 12 months and younger (infants), especially premature infants, older adults, people with heart and lung disease, particularly congenital heart disease or chronic obstructive pulmonary (COPD), or anyone with a weak immune system (immunocompromised), asthma, or cancer, or who had a lung transplant.

In certain embodiments, treatment is administered as peri-exposure or pre-exposure prophylaxis.

In therapeutic settings, in contrast, the subject is typically infected with a RSV and/or a MPV, diagnosed with RSV and/or MPV infection, and/or showing symptoms of RSV and/or MPV infection. Of note, the terms “treatment” and “therapy”/“therapeutic” of RSV and/or MPV infection include (complete) cure as well as attenuation/reduction of RSV and/or MPV infection and/or related symptoms (e.g., attenuation/reduction of severity of infection and/or symptoms, number of symptoms, duration of infection and/or symptoms, or any combination thereof).

It will be understood that reference herein to a reduced number and/or severity of symptoms, which reduction results from administration of a presently disclosed pharmaceutical composition, describes a comparison with a reference subject who did not receive a disclosed pharmaceutical composition. A reference subject can be, for example, (i) the same subject during an earlier period of time (e.g., a prior RSV and/or MPV season), (ii) a subject of a same or a similar: age or age group; gender; pregnancy status; chronic medical condition (such as chronic cardiac, pulmonary, renal, metabolic, neurodevelopmental, liver or hematologic diseases) or lack thereof, and/or immunosuppressive condition or lack thereof, or (iii) a typical subject within a population (e.g., local, regional, or national, including of a same or similar age or age range and/or general state of health) during a RSV and/or MPV virus season.

Prophylaxis can be determined by, for example, the failure to develop a diagnosed RSV and/or MPV infection and/or the lack of symptoms associated with RSV and/or MPV infection during a part of a full RSV and/or MPV season, or over a full RSV and/or MPV season.

In certain embodiments, the methods provided herein include administering a therapeutically effective amount of a composition according to the present disclosure to a subject at immediate risk of RSV and/or MPV infection.

Typical routes of administering the presently disclosed compositions include, without limitation, oral, topical, transdermal, inhalation, parenteral, sublingual, buccal, rectal, vaginal, and intranasal. The term “parenteral”, as used herein, includes subcutaneous injections, intravenous, intramuscular, intrasternal injection or infusion techniques. In certain embodiments, administering comprises administering by a route that is selected from oral, intravenous, parenteral, intragastric, intrapleural, intrapulmonary, intrarectal, intradermal, intraperitoneal, intratumoral, subcutaneous, topical, transdermal, intracisternal, intrathecal, intranasal, and intramuscular. In particular embodiments, a method comprises orally administering the antibody, antigen-binding fragment, polynucleotide, vector, host cell, or composition to the subject.

Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described an antibody or antigen-binding in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain an effective amount of an antibody or antigen-binding fragment, polynucleotide, vector, host cell, or composition of the present disclosure, for treatment of a disease or condition of interest in accordance with teachings herein.

A composition may be in the form of a solid or liquid. In some embodiments, the carrier(s) are particulate, so that the compositions are, for example, in tablet or powder form.

The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi solid, semi liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the composition is in the form of a capsule, for example, a gelatin capsule, it may contain, in addition to materials of the above type, a liquid carrier such as polyethylene glycol or oil.

The composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more ofthe following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of an antibody or antigen-binding fragment as herein disclosed such that a suitable dosage will be obtained. Typically, this amount is at least 0.01% of the antibody or antigen-binding fragment in the composition. When intended for oral administration, this amount may be varied to be between 0.1 and about 70% of the weight of the composition.

Certain oral pharmaceutical compositions contain between about 4% and about 75% of the antibody or antigen-binding fragment. In certain embodiments, pharmaceutical compositions and preparations according to the present invention are prepared so that a parenteral dosage unit contains between 0.01 to 10% by weight of antibody or antigen-binding fragment prior to dilution.

The composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient.

Such bases include, without limitation, lanolin, cocoa butter and polyethylene glycol.

A composition may include various materials which modify the physical form of a solid or liquid dosage unit. For example, the composition may include materials that form a coating shell around the active ingredients. The materials that form the coating shell are typically inert, and may be selected from, for example, sugar, shellac, and other enteric coating agents.

Alternatively, the active ingredients may be encased in a gelatin capsule. The composition in solid or liquid form may include an agent that binds to the antibody or antigen-binding fragment of the disclosure and thereby assists in the delivery of the compound. Suitable agents that may act in this capacity include monoclonal or polyclonal antibodies, one or more proteins or a liposome. The composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi phasic, or tri phasic systems in order to deliver the active ingredient(s). Delivery of the aerosol includes the necessary container, activators, valves, subcontainers, and the like, which together may form a kit. One of ordinary skill in the art, without undue experimentation, may determine preferred aerosols.

It will be understood that compositions of the present disclosure also encompass carrier molecules for polynucleotides, as described herein (e.g., lipid nanoparticles, nanoscale delivery platforms, and the like).

The pharmaceutical compositions may be prepared by methodology well known in the pharmaceutical art. For example, a composition intended to be administered by injection can be prepared by combining a composition that comprises an antibody, antigen-binding fragment, or antibody conjugate as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the peptide composition so as to facilitate dissolution or homogeneous suspension of the antibody or antigen-binding fragment in the aqueous delivery system.

In general, an appropriate dose and treatment regimen provide the composition(s) in an amount sufficient to provide therapeutic and/or prophylactic benefit (such as described herein, including an improved clinical outcome (e.g., a decrease in frequency, duration, or severity of diarrhea or associated dehydration, or inflammation, or longer disease-free and/or overall survival, or a lessening of symptom severity). For prophylactic use, a dose should be sufficient to prevent, delay the onset of, or diminish the severity of a disease associated with disease or disorder. Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Compositions are administered in an effective amount (e.g., to treat and/or prevent RSV and/or MPV infection), which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In certain embodiments, following administration of therapies according to the formulations and methods of this disclosure, test subjects will exhibit about a 10% up to about a 99% reduction in one or more symptoms associated with the disease or disorder being treated as compared to placebo-treated or other suitable control subjects.

Generally, a therapeutically effective dose of an antibody or antigen binding fragment is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g). For polynucleotides, vectors, host cells, and related compositions of the present disclosure, a therapeutically effective dose may be different than for an antibody or antigen-binding fragment.

In certain embodiments, a method comprises administering the antibody, antigen-binding fragment, polynucleotide, vector, host cell, or composition to the subject at 2, 3, 4, 5, 6, 7, 8, 9, 10 times, or more.

In certain embodiments, a method comprises administering the antibody, antigen-binding fragment, or composition to the subject a plurality of times, wherein a second or successive administration is performed at about 6, about 7, about 8, about 9, about 10, about 11, about 12, about 24, about 48, about 74, about 96 hours, or more, following a first or prior administration, respectively.

In certain embodiments, a method comprises administering the antibody, antigen-binding fragment, polynucleotide, vector, host cell, or composition one or more time prior to the subject being infected by RSV and/or MPV.

Compositions comprising an antibody, antigen-binding fragment, polynucleotide, vector, host cell, or composition of the present disclosure may also be administered simultaneously with, prior to, or after administration of one or more other therapeutic agents, such as, for example, an antiviral, e.g., ribavirin or a monoclonal antibody, e.g. palivizumab, nirsevimab, or clesrovimab, or an antibody having the VH and VL of any one of these antibodies. Such combination therapy may include administration of a single pharmaceutical dosage formulation which contains a compound of the invention and one or more additional active agents, as well as administration of compositions comprising an antibody or antigen-binding fragment of the disclosure and each active agent in its own separate dosage formulation. For example, an antibody or antigen-binding fragment as described herein and the other active agent can be administered to the patient together in a single oral dosage composition such as a tablet or capsule, or each agent administered in separate oral dosage formulations. Similarly, an antibody or antigen-binding fragment as described herein and the other active agent can be administered to the subject together in a single parenteral dosage composition such as in a saline solution or other physiologically acceptable solution, or each agent administered in separate parenteral dosage formulations. Where separate dosage formulations are used, the compositions comprising an antibody or antigen-binding fragment and one or more additional active agents can be administered at essentially the same time, i.e., concurrently, or at separately staggered times, i.e., sequentially and in any order; combination therapy is understood to include all these regimens.

In some embodiments, an antibody (or one or more nucleic acid, host cell, vector, or composition) is administered to a subject who has previously received one or more anti-inflammatory agent and/or one or more antiviral agent. In some embodiments, the antiviral is ribavirin. In some embodiments, one or more anti-inflammatory agent and/or one or more antiviral agent is administered to a subject who has previously received an antibody (or one or more nucleic acid, host cell, vector, or composition). In some embodiments, the antiviral is a ribavirin.

In some embodiments, an antibody, polynucleotide, vector, or composition as described herein is administered to prevent or treat a RSV infection only, or a MPV infection only. In such embodiments, the antibody may specifically bind only RSV or only MPV.

In a related aspect, uses of the presently disclosed antibodies, antigen-binding fragments, vectors, host cells, and compositions (e.g., in the diagnosis, prophylaxis, and/or treatment of RSV and/or MPV infection, in the manufacture of a medicament for preventing or treating RSV and/or MPV infection) are provided.

In certain embodiments, an antibody, antigen-binding fragment, polynucleotide, vector, host cell, or composition is provided for use in a method of treating a RSV and/or MPV infection in a subject. In some embodiments, the method of treating is a method of treating only RSV infection or MPV infection, not infection by both viruses. Antibodies identified herein as binding only RSV or MPV and any related antigen-binding fragment, polynucleotide, vector, host cell, or composition may in particular be used in such embodiments.

In certain embodiments, an antibody, antigen-binding fragment, or composition is provided for use in a method of manufacturing or preparing a medicament for treating RSV and/or MPV infection in a subject. In some embodiments, the medicament is for treating only RSV infection or MPV infection, not infection by both viruses. Antibodies identified herein as binding only RSV or MPV and any related antigen-binding fragment, polynucleotide, vector, host cell, or composition may in particular be used in such embodiments.

The present disclosure further provides a kit comprising one or more of any antibodies, antigen-binding fragments, polynucleotides, nucleic acids, vectors, or other compositions disclosed herein. The kit may further include one or more of a container, such as a tube, vial, or syringe, an activator, a valve, a subcontainer, or instructions for use, such as for administering to a subject.

The present disclosure also provides the following exemplary embodiments: Embodiment 1. An antibody or antigen-binding fragment comprising a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are according to the IMGT numbering system, wherein the antibody or antigen-binding fragment binds to a respiratory syncytial virus (RSV) fusion glycoprotein (RSV-F) and wherein: (i) the CDRH1 comprises or consists of the amino acid sequence set forth in any one of SEQ ID NOs.: 3, 137, 147, 160, 170, 182, 216, 234, 244, 262, 278, 285, 301, 333, 363, 370, 728, 738, 747, 756, 766, 776, 785, 795, 805, 814, 887, 890, 13, 23, 31, 39, 54, 60, 74, 80, 87, 112, 121, 130, 380, 387, 396, 417, 441, 452, 459, 471, 481, 492, 498, 505, 514, 528, and 533 or a functional variant thereof comprising one, two, or three acid substitutions, one or more of which substitutions is optionally a conservative substitution and/or is a substitution to a germline-encoded amino acid; (ii) the CDRH2 comprises or consists of the amino acid sequence set forth in any one of SEQ ID NOs.: 4, 138, 161, 217, 226, 235, 263, 279, 302, 334, 364, 371, 729, 739, 748, 757, 767, 777, 786, 796, 815, 818, 838, 884, 894, 897, 14, 24, 32, 40, 47, 61, 81, 88, 96, 113, 122, 388, 397, 405, 442, 453, 464, 506, 515, 534, 540, 543, and 548 or a functional variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence set forth in any one of SEQ ID NOs.: 5, 139, 148, 162, 176, 183, 190, 197, 203, 218, 227, 236, 245, 255, 264, 272, 280, 286, 294, 303, 310, 317, 322, 328, 335, 343, 353, 358, 365, 372, 730, 740, 749, 758, 768, 778, 787, 797, 806, 821, 824, 832, 835, 841, 844, 15, 25, 33, 48, 62, 89, 114, 123, 131, 389, 398, 406, 427, 432, 476, 493, 499, 507, 516, 535, and 551 or a functional variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence set forth in any one of SEQ ID NOs.: 8, 142, 151, 165, 173, 186, 193, 206, 221, 230, 239, 258, 267, 289, 297, 306, 313, 338, 350, 375, 733, 743, 761, 771, 781, 790, 800, 809, 18, 65, 92, 99, 126, 383, 401, 437, 445, 456, 488, 554, 561, 564, and 568 or a functional variant thereof comprising one, two, or three amino acid substitutions, one or more of which substitutions is optionally a conservative substitution and/or is a substitution to a germline-encoded amino acid; (v) the CDRL2 comprises or consists of the amino acid sequence set forth in any one of SEQ ID NOs.: 9, 143, 152, 156, 166, 200, 207, 222, 240, 268, 290, 314, 339, 734, 752, 762, 772, 791, 801, 856, 871, 19, 43, 66, 117, 392, 409, 467, 489, 510, 519, 555, and 705, or a functional variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence set forth in any one of SEQ ID NOs.: 10, 144, 153, 157, 167, 179, 187, 194, 208, 213, 223, 231, 241, 248, 259, 269, 275, 291, 298, 307, 325, 340, 376, 735, 744, 753, 763, 773, 782, 792, 802, 810, 848, 852, 866, 20, 28, 36, 44, 51, 57, 67, 77, 84, 93, 118, 127, 134, 384, 393, 402, 410, 420, 438, 446, 468, 502, 511, 556, and 520 or a functional variant thereof comprising having one, two, or three amino acid substitutions, one or more of which substitutions is optionally a conservative substitution and/or is a substitution to a germline-encoded amino acid.

Embodiment 2. An antibody or antigen-binding fragment comprising a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are according to the IMGT numbering system, wherein the antibody or antigen-binding fragment binds to a respiratory syncytial virus (RSV) fusion glycoprotein (RSV-F) and wherein: (i) the CDRH1 comprises or consists of the amino acid sequence set forth in any one of SEQ ID NOs.: 3, 137, 147, 160, 170, 182, 216, 234, 244, 262, 278, 285, 301, 333, 363, 370, 728, 738, 747, 756, 766, 776, 785, 795, 805, 814, 887, and 890 or a functional variant thereof comprising one, two, or three acid substitutions, one or more of which substitutions is optionally a conservative substitution and/or is a substitution to a germline-encoded amino acid; (ii) the CDRH2 comprises or consists of the amino acid sequence set forth in any one of SEQ ID NOs.: 4, 138, 161, 217, 226, 235, 263, 279, 302, 334, 364, 371, 729, 739, 748, 757, 767, 777, 786, 796, 815, 818, 838, 884, 894, and 897 or a functional variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence set forth in any one of SEQ ID NOs.: 5, 139, 148, 162, 176, 183, 190, 197, 203, 218, 227, 236, 245, 255, 264, 272, 280, 286, 294, 303, 310, 317, 322, 328, 335, 343, 353, 358, 365, 372, 730, 740, 749, 758, 768, 778, 787, 797, 806, 821, 824, 832, 835, 841, and 844 or a functional variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence set forth in any one of SEQ ID NOs.: 8, 142, 151, 165, 173, 186, 193, 206, 221, 230, 239, 258, 267, 289, 297, 306, 313, 338, 350, 375, 733, 743, 761, 771, 781, 790, 800, and 809 or a functional variant thereof comprising one, two, or three amino acid substitutions, one or more of which substitutions is optionally a conservative substitution and/or is a substitution to a germline-encoded amino acid; (v) the CDRL2 comprises or consists of the amino acid sequence set forth in any one of SEQ ID NOs.: 9, 143, 152, 156, 166, 200, 207, 222, 240, 268, 290, 314, 339, 734, 752, 762, 772, 791, 801, 856, and 871 or a functional variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence set forth in any one of SEQ ID NOs.: 10, 144, 153, 157, 167, 179, 187, 194, 208, 213, 223, 231, 241, 248, 259, 269, 275, 291, 298, 307, 325, 340, 376 735, 744, 753, 763, 773, 782, 792, 802, 810, 848, 852, and 866 or a functional variant thereof comprising having one, two, or three amino acid substitutions, one or more of which substitutions is optionally a conservative substitution and/or is a substitution to a germline-encoded amino acid.

Embodiment 3. The antibody or antigen-binding fragment of embodiment 1 or embodiment 2, comprising CDRH1, CDRH2, CDRH3, CDRL1, CDRL2, and CDRL3 amino acid sequences set forth in SEQ ID NOs.: 1) SEQ ID NOs.: 3-5 and 8-10; 2) SEQ ID NOs.: 137-139 and 142-144; 3) SEQ ID NOs.: 147, 138, 148, and 151-153; 4) SEQ ID NOs.: 147, 138, 148, 142, 156, and 157; 5) SEQ ID NOs.: 160-162 and 165-167; 6) SEQ ID NOs.: 170, 138, 148, 173, 156, and 157; 7) SEQ ID NOs.: 137, 138, 176, 142, 143, and 179; 8) SEQ ID NOs.: 182, 138, 183, 186, 143, and 187; 9) SEQ ID NOs.: 137, 138, 190, 193, 143, 194; 10) SEQ ID NOs.: 137, 138, 197, 142, 200, and 157; 11) SEQ ID NOs.: 137, 138, 203, and 206-208; 12) SEQ ID NOs.: 137, 138, 203, 142, 143, and 213; 13) SEQ ID NOs.: 216-218 and 221-223; 14) SEQ ID NOs.: 137, 226, 227,230, 143, and 231; 15) SEQ ID NOs.: 234-236 and 239-241; 16) SEQ ID NOs.: 244, 138, 245, 173, 143, and 248; 17) SEQ ID NOs.: 182, 138, 148, 142, 156, and 157; 18) SEQ ID NOs.: 137, 138, 255, 258, 156, and 259; 19) SEQ ID NOs.: 262-264 and 267-269; 20) SEQ ID NOs.: 137, 138, 272, 173, 143, and 275; 21) SEQ ID NOs.: 278-280 and 206-208; 22) SEQ ID NOs.: 285, 138, 286, and 289-291; 23) SEQ ID NOs.: 137, 226, 294, 297, 143, and 298; 24) SEQ ID NOs.: 301-303, 306, 268, and 307; 25) SEQ ID NOs.: 137, 138, 310, 313, 314, and 157; 26) 137,138, 317, 142, 143, and 213; 27) SEQ ID NOs.: 137, 138, 322, 142, 143, and 325; 28) SEQ ID NOs.: 137, 138, 322, 142,143, and 325; 29) SEQ ID NOs.: 170, 138, 328, 142, 143, and 157; 30) SEQ ID NOs.: 333-335 and 338-340; 31) SEQ ID NOs.: 137, 138, 343, 173, 143, and 213; 32) SEQ ID NOs.: 301-303, 350, 268, and 307; 33) SEQ ID NOs.: 137, 138, 353, 142, 143, and 213; 34) SEQ ID NOs.: 137, 226, 358, 142, 143, and 231; 35) SEQ ID NOs.: 363-365 and 8-10; 36) SEQ ID NOs.: 370-372, 375, 143, and 376, 37) SEQ ID NOs.: 738, 884, 740, 743, 240, and 744; 38) SEQ ID NOs.: 738-740, 743, 240, and 744; 39) SEQ ID NOs.: 728-730, and 733-735; 40) SEQ ID NOs.: 747-749, 221, and 752-753; 41) SEQ ID NOs.: 756-758, and 761-763; 42) SEQ ID NOs.: 766-768, and 771-773; 43) SEQ ID NOs.: 776-778, 781, 268, and 782; 44) SEQ ID NOs.: 785-787, and 790-792; 45) SEQ ID NOs.: 795-797, and 800-802; 46) SEQ ID NOs.: 805, 796, 806, 809, 801, and 810; 47) SEQ ID NOs.: 234, 838, 236, and 239-241; 48) SEQ ID NOs.: 137, 226, 358, 142, 143, and 848; 49) SEQ ID NOs.: 814, 226, 358, 142, 143, and 231; 50) SEQ ID NOs.: 137-139, 142, 143, and 852; 51) SEQ ID NOs.: 814, 818, 139 and 142-144; 52) 738, 884, 740, 743, 240, and 744; 53) SEQ ID NOs.: 887, 884, 740, 743, 240, and 744; or 54) SEQ ID NOs.: 738, 884, 740, 743, 240, and 744, respectively.

Embodiment 4. The antibody or antigen-binding fragment of any one of embodiments 1-3, wherein: (i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 2, 136, 146, 159, 169, 175, 181, 189, 196, 202, 210, 215, 225, 233, 243, 250, 254, 261, 271, 277, 284, 293, 300, 309, 316, 321, 327, 332, 342, 347, 352, 357, 362, 369, 727, 737, 746, 755, 765, 775, 784, 794, 804, 813, 817, 820, 823, 826, 828, 831, 834, 837, 840, 843, 883, 886, 889, 893, 896, 899, 901, 903, 129, 12, 38, 46, 53, 59, 69, 120, 73, 79, 86, 95, 22, 111, 30, 378, 386, 395, 404, 412, 416, 422, 426, 431, 440, 451, 458, 463, 470, 475, 480, 485, 491, 497, 532, 537, 539, 542, 545, 547, 550, 504, 513, 524, 527, 702, 707, 712, and 716, wherein sequence variation is optionally limited to one or more framework regions and/or sequence variation comprises one or more substitution to a germline-encoded amino acid; and/or (ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 7, 141, 150, 155, 164, 172, 178, 185, 192, 199, 205, 212, 220, 229, 238, 247, 252, 257, 266, 274, 282, 288, 296, 305, 312, 319, 324, 330, 337, 345, 349, 355, 360, 367, 374, 732, 742, 751, 760, 770, 780, 789, 799, 808, 847, 851, 855, 858, 860, 862, 865, 868, 870, 873, 875, 877, 879, 881, 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 553, 558, 560, 563, 567, 570, 572, 574, 704, and 710, wherein sequence variation is optionally limited to one or more framework regions and/or sequence variation comprises one or more substitution to a germline-encoded amino acid.

Embodiment 5. The antibody or antigen-binding fragment of any one of embodiments 1-4, wherein: (i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 2, 136, 146, 159, 169, 175, 181, 189, 196, 202, 210, 215, 225, 233, 243, 250, 254, 261, 271, 277, 284, 293, 300, 309, 316, 321, 327, 332, 342, 347, 352, 357, 362, 369, 727, 737, 746, 755, 765, 775, 784, 794, 804, 813, 817, 820, 823, 826, 828, 831, 834, 837, 840, 843, 883, 886, 889, 893, 896, 899, 901, and 903 wherein sequence variation is optionally limited to one or more framework regions and/or sequence variation comprises one or more substitution to a germline-encoded amino acid; and/or (ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID Nos.: 7, 141, 150, 155, 164, 172, 178, 185, 192, 199, 205, 212, 220, 229, 238, 247, 252, 257, 266, 274, 282, 288, 296, 305, 312, 319, 324, 330, 337, 345, 349, 355, 360, 367, 374, 732, 742, 751, 760, 770, 780, 789, 799, 808, 847, 851, 855, 858, 860, 862, 865, 868, 870, 873, 875, 877, 879, and 881 wherein sequence variation is optionally limited to one or more framework regions and/or sequence variation comprises one or more substitution to a germline-encoded amino acid.

Embodiment 6. The antibody or antigen-binding fragment of any one of embodiments 1-5, wherein:
(1) the VH and the VL comprise or consist of amino acid sequences having at least 90%, at least 91%, at least 92%, at least 93%, at least 94%, at least 95%, at least 96%, at least 97%, at least 98%, or at least 99% identity to the amino acid sequences set forth in: (i) SEQ ID NOs.: 702 and 704, respectively; (ii) SEQ ID NOs.:136 and 851, respectively; (iii) SEQ ID NOs.: 817 and 141, respectively; (iv) SEQ ID NOs.: 899 and 360, respectively; (v) SEQ ID NOs.: 136 and 851, respectively; (vi) SEQ ID NOs.: 817 and 141, respectively; (vii) SEQ ID NOs.: 233 and 858, respectively; (viii) SEQ ID NOs.: 837 and 858, respectively; (ix) SEQ ID NOs.: 357 and 847, respectively, wherein sequence variation is optionally limited to one or more framework regions and/or sequence variation comprises one or more substitution to a germline-encoded amino acid;
(2) the antibody or antigen-binding fragment comprises the CDRH1, CDRH2, CDRH3, CDRL1, CDRL2, and CDRL3 amino acid sequences of the VH and VL amin oacid sequences set forth in(i) SEQ ID NOs.: 702 and 704, respectively; (ii) SEQ ID NOs.:136 and 851, respectively; (iii) SEQ ID NOs.: 817 and 141, respectively; (iv) SEQ ID NOs.: 899 and 360, respectively; (v) SEQ ID NOs.: 136 and 851, respectively; (vi) SEQ ID NOs.: 817 and 141, respectively; (vii) SEQ ID NOs.: 233 and 858, respectively; (viii) SEQ ID NOs.: 837 and 858, respectively; (ix) SEQ ID NOs.: 357 and 847, respectively; or
(3)(i) the CDRH1 comprises or consists of the amino acid sequence set forth in any one of SEQ ID NOs.: 121, 137, 814, 234, 814, or a functional variant thereof comprising one, two, or three acid substitutions, one or more of which substitutions is optionally a conservative substitution and/or is a substitution to a germline-encoded amino acid; (ii) the CDRH2 comprises or consists of the amino acid sequence set forth in any one of SEQ ID NOs.: 122, 138, 818, 235, 838, 226, or a functional variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence set forth in any one of SEQ ID NOs.: 123, 139, 236, 358, or a functional variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence set forth in any one of SEQ ID NOs.: 18, 142, 239, or a functional variant thereof comprising one, two, or three amino acid substitutions, one or more of which substitutions is optionally a conservative substitution and/or is a substitution to a germline-encoded amino acid; (v) the CDRL2 comprises or consists of the amino acid sequence set forth in any one of SEQ ID NOs.: 705, 143, 240, or a functional variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence set forth in any one of SEQ ID NOs.: 127, 852, 144, 241, 848, 231, or a functional variant thereof comprising having one, two, or three amino acid substitutions, one or more of which substitutions is optionally a conservative substitution and/or is a substitution to a germline-encoded amino acid.

Embodiment 7. The antibody or antigen-binding fragment of any one of embodiments 1-6, wherein the VH and the VL comprise or consist of amino acid sequences having at least 90%, at least 91%, at least 92%, at least 93%, at least 94%, at least 95%, at least 96%, at least 97%, at least 98%, or at least 99% identity to the amino acid sequences set forth in: SEQ ID NOs.: 136 and 851, respectively, wherein sequence variation is optionally limited to one or more framework regions and/or sequence variation comprises one or more substitution to a germline-encoded amino acid.

Embodiment 8. The antibody or antigen-binding fragment of any one of embodiments 1-7, wherein the antibody or antigen-binding fragment comprises the CDRH1, CDRH2, CDRH3, CDRL1, CDRL2, and CDRL3 amino acid sequences of the VH and VL amino acid sequences set forth in (i) SEQ ID NOs.: 136 and 851, respectively.

Embodiment 9. The antibody or antigen-binding fragment of any one of embodiments 1-8, wherein (i) the CDRH1 comprises or consists of the amino acid sequence set forth in SEQ ID NO.: 137, or a functional variant thereof comprising one, two, or three acid substitutions, one or more of which substitutions is optionally a conservative substitution and/or is a substitution to a germline-encoded amino acid; (ii) the CDRH2 comprises or consists of the amino acid sequence set forth in SEQ ID NO.: 138, or a functional variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence set forth in SEQ ID NO.: 139, or a functional variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence set forth in SEQ ID NO.: 142, or a functional variant thereof comprising one, two, or three amino acid substitutions, one or more of which substitutions is optionally a conservative substitution and/or is a substitution to a germline-encoded amino acid; (v) the CDRL2 comprises or consists of the amino acid sequence set forth in SEQ ID NO.: 143, or a functional variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence set forth in SEQ ID NO.: 852, or a functional variant thereof comprising having one, two, or three amino acid substitutions, one or more of which substitutions is optionally a conservative substitution and/or is a substitution to a germline-encoded amino acid.

Embodiment 10. The antibody or antigen-binding fragment of any one of embodiments 1-6, wherein the VH and the VL comprise or consist of amino acid sequences having at least 90%, at least 91%, at least 92%, at least 93%, at least 94%, at least 95%, at least 96%, at least 97%, at least 98%, or at least 99% identity to the amino acid sequences set forth in: SEQ ID NOs.: 817 and 141, respectively, wherein sequence variation is optionally limited to one or more framework regions and/or sequence variation comprises one or more substitution to a germline-encoded amino acid.

Embodiment 11. The antibody or antigen-binding fragment of any one of embodiments 1-6 and 10, wherein the antibody or antigen-binding fragment comprises the CDRH1, CDRH2, CDRH3, CDRL1, CDRL2, and CDRL3 amino acid sequences of the VH and VL amino acid sequences set forth in (i) SEQ ID NOs.: 817 and 141, respectively.

Embodiment 12. The antibody or antigen-binding fragment of any one of embodiments 1-6 and 10⁻¹¹, wherein (i) the CDRH1 comprises or consists of the amino acid sequence set forth in SEQ ID NO.: 814, or a functional variant thereof comprising one, two, or three acid substitutions, one or more of which substitutions is optionally a conservative substitution and/or is a substitution to a germline-encoded amino acid; (ii) the CDRH2 comprises or consists of the amino acid sequence set forth in SEQ ID NO.: 818, or a functional variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence set forth in SEQ ID NO.: 139, or a functional variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence set forth in SEQ ID NO.: 142, or a functional variant thereof comprising one, two, or three amino acid substitutions, one or more of which substitutions is optionally a conservative substitution and/or is a substitution to a germline-encoded amino acid; (v) the CDRL2 comprises or consists of the amino acid sequence set forth in SEQ ID NO.: 143, or a functional variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence set forth in SEQ ID NO.: 144, or a functional variant thereof comprising having one, two, or three amino acid substitutions, one or more of which substitutions is optionally a conservative substitution and/or is a substitution to a germline-encoded amino acid.

Embodiment 13. The antibody or antigen-binding fragment of any one of embodiments 1-6, wherein the VH and the VL comprise or consist of amino acid sequences having at least 90%, at least 91%, at least 92%, at least 93%, at least 94%, at least 95%, at least 96%, at least 97%, at least 98%, or at least 99% identity to the amino acid sequences set forth in: SEQ ID NOs.: 233 and 858, respectively, wherein sequence variation is optionally limited to one or more framework regions and/or sequence variation comprises one or more substitution to a germline-encoded amino acid.

Embodiment 14. The antibody or antigen-binding fragment of any one of embodiments 1-6 and 13, wherein the antibody or antigen-binding fragment comprises the CDRH1, CDRH2, CDRH3, CDRL1, CDRL2, and CDRL3 amino acid sequences of the VH and VL amino acid sequences set forth in (i) SEQ ID NOs.: 233 and 858, respectively. Embodiment 15. The antibody or antigen-binding fragment of any one of embodiments 1-6 and 13-14, wherein (i) the CDRH1 comprises or consists of the amino acid sequence set forth in SEQ ID NO.: 234, or a functional variant thereof comprising one, two, or three acid substitutions, one or more of which substitutions is optionally a conservative substitution and/or is a substitution to a germline-encoded amino acid; (ii) the CDRH2 comprises or consists of the amino acid sequence set forth in SEQ ID NO.: 235, or a functional variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence set forth in SEQ ID NO.: 236, or a functional variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence set forth in SEQ ID NO.: 239, or a functional variant thereof comprising one, two, or three amino acid substitutions, one or more of which substitutions is optionally a conservative substitution and/or is a substitution to a germline-encoded amino acid; (v) the CDRL2 comprises or consists of the amino acid sequence set forth in SEQ ID NO.: 240, or a functional variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence set forth in SEQ ID NO.: 241, or a functional variant thereof comprising having one, two, or three amino acid substitutions, one or more of which substitutions is optionally a conservative substitution and/or is a substitution to a germline-encoded amino acid.

Embodiment 16. The antibody or antigen-binding fragment of any one of embodiments 1-6, wherein the VH and the VL comprise or consist of amino acid sequences having at least 90%, at least 91%, at least 92%, at least 93%, at least 94%, at least 95%, at least 96%, at least 97%, at least 98%, or at least 99% identity to the amino acid sequences set forth in: SEQ ID NOs.: 837 and 858, respectively, wherein sequence variation is optionally limited to one or more framework regions and/or sequence variation comprises one or more substitution to a germline-encoded amino acid.

Embodiment 17. The antibody or antigen-binding fragment of any one of embodiments 1-6 and 16, wherein the antibody or antigen-binding fragment comprises the CDRH1, CDRH2, CDRH3, CDRL1, CDRL2, and CDRL3 amino acid sequences of the VH and VL amino acid sequences set forth in (i) SEQ ID NOs.: 837 and 858, respectively.

Embodiment 18. The antibody or antigen-binding fragment of any one of embodiments 1-6 and 16-17, wherein (i) the CDRH1 comprises or consists of the amino acid sequence set forth in SEQ ID NO.: 234, or a functional variant thereof comprising one, two, or three acid substitutions, one or more of which substitutions is optionally a conservative substitution and/or is a substitution to a germline-encoded amino acid; (ii) the CDRH2 comprises or consists of the amino acid sequence set forth in SEQ ID NO.: 838, or a functional variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence set forth in SEQ ID NO.: 236, or a functional variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence set forth in SEQ ID NO.: 239, or a functional variant thereof comprising one, two, or three amino acid substitutions, one or more of which substitutions is optionally a conservative substitution and/or is a substitution to a germline-encoded amino acid; (v) the CDRL2 comprises or consists of the amino acid sequence set forth in SEQ ID NO.: 240, or a functional variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence set forth in SEQ ID NO.: 241, or a functional variant thereof comprising having one, two, or three amino acid substitutions, one or more of which substitutions is optionally a conservative substitution and/or is a substitution to a germline-encoded amino acid.

Embodiment 19. The antibody or antigen-binding fragment of any one of embodiments 1-6, wherein the VH and the VL comprise or consist of amino acid sequences having at least 90%, at least 91%, at least 92%, at least 93%, at least 94%, at least 95%, at least 96%, at least 97%, at least 98%, or at least 99% identity to the amino acid sequences set forth in: SEQ ID NOs.: 357 and 847, respectively, wherein sequence variation is optionally limited to one or more framework regions and/or sequence variation comprises one or more substitution to a germline-encoded amino acid.

Embodiment 20. The antibody or antigen-binding fragment of any one of embodiments 1-6 and 19, wherein the antibody or antigen-binding fragment comprises the CDRH1, CDRH2, CDRH3, CDRL1, CDRL2, and CDRL3 amino acid sequences of the VH and VL amino acid sequences set forth in (i) SEQ ID NOs.: 357 and 847, respectively.

Embodiment 21. The antibody or antigen-binding fragment of any one of embodiments 1-6 and 19-20, wherein (i) the CDRH1 comprises or consists of the amino acid sequence set forth in SEQ ID NO.: 137, or a functional variant thereof comprising one, two, or three acid substitutions, one or more of which substitutions is optionally a conservative substitution and/or is a substitution to a germline-encoded amino acid; (ii) the CDRH2 comprises or consists of the amino acid sequence set forth in SEQ ID NO.: 226, or a functional variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence set forth in SEQ ID NO.: 358, or a functional variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence set forth in SEQ ID NO.: 142, or a functional variant thereof comprising one, two, or three amino acid substitutions, one or more of which substitutions is optionally a conservative substitution and/or is a substitution to a germline-encoded amino acid; (v) the CDRL2 comprises or consists of the amino acid sequence set forth in SEQ ID NO.: 143, or a functional variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence set forth in SEQ ID NO.: 848, or a functional variant thereof comprising having one, two, or three amino acid substitutions, one or more of which substitutions is optionally a conservative substitution and/or is a substitution to a germline-encoded amino acid.

Embodiment 22. The antibody or antigen-binding fragment of any one of embodiments 1-6, wherein the VH and the VL comprise or consist of amino acid sequences having at least 90%, at least 91%, at least 92%, at least 93%, at least 94%, at least 95%, at least 96%, at least 97%, at least 98%, or at least 99% identity to the amino acid sequences set forth in: SEQ ID NOs.: 899 and 360, respectively, wherein sequence variation is optionally limited to one or more framework regions and/or sequence variation comprises one or more substitution to a germline-encoded amino acid.

Embodiment 23. The antibody or antigen-binding fragment of any one of embodiments 1-6 and 22, wherein the antibody or antigen-binding fragment comprises the CDRH1, CDRH2, CDRH3, CDRL1, CDRL2, and CDRL3 amino acid sequences of the VH and VL amino acid sequences set forth in (i) SEQ ID NOs.: 899 and 360, respectively.

Embodiment 24. The antibody or antigen-binding fragment of any one of embodiments 1-6 and 22-23, wherein (i) the CDRH1 comprises or consists of the amino acid sequence set forth in SEQ ID NO.: 814, or a functional variant thereof comprising one, two, or three acid substitutions, one or more of which substitutions is optionally a conservative substitution and/or is a substitution to a germline-encoded amino acid; (ii) the CDRH2 comprises or consists of the amino acid sequence set forth in SEQ ID NO.: 226, or a functional variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence set forth in SEQ ID NO.: 358, or a functional variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence set forth in SEQ ID NO.: 142, or a functional variant thereof comprising one, two, or three amino acid substitutions, one or more of which substitutions is optionally a conservative substitution and/or is a substitution to a germline-encoded amino acid; (v) the CDRL2 comprises or consists of the amino acid sequence set forth in SEQ ID NO.: 143, or a functional variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence set forth in SEQ ID NO.: 231, or a functional variant thereof comprising having one, two, or three amino acid substitutions, one or more of which substitutions is optionally a conservative substitution and/or is a substitution to a germline-encoded amino acid.

Embodiment 25. An antibody or antigen-binding fragment comprising a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are according to the IMGT numbering system, wherein the antibody or antigen-binding fragment binds to a metapneumovirus (MPV) fusion glycoprotein (MPV-F) and wherein: (i) the CDRH1 comprises or consists of the amino acid sequence set forth in any one of SEQ ID Nos.: 102, 13, 23, 31, 39, 54, 60, 74, 80, 87, 112, 121, 130, 380, 387, 396, 417, 441, 452, 459, 471, 481, 492, 498, 505, 514, 528, and 533 or a functional variant thereof comprising one, two, or three acid substitutions, one or more of which substitutions is optionally a conservative substitution and/or is a substitution to a germline-encoded amino acid; (ii) the CDRH2 comprises or consists of the amino acid sequence set forth in any one of SEQ ID Nos.: 103, 14, 24, 32, 40, 47, 61, 81, 88, 96, 113, 122, 388, 397, 405, 442, 453, 464, 506, 515, 534, 540, 543, and 548 or a functional variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence set forth in any one of SEQ ID NO.: 104, 15, 25, 33, 48, 62, 89, 114, 123, 131, 389, 398, 406, 427, 432, 476, 493, 499, 507, 516, 535, and 551 or a functional variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence set forth in any one of SEQ ID Nos.: 107, 18, 65, 92, 99, 126, 383, 401, 437, 445, 456, 488, 554, 561, 564, and 568 or a functional variant thereof comprising one, two, or three amino acid substitutions, one or more of which substitutions is optionally a conservative substitution and/or is a substitution to a germline-encoded amino acid; (v) the CDRL2 comprises or consists of the amino acid sequence set forth in any one of SEQ ID Nos.: 108, 19, 43, 66, 117, 392, 409, 467, 489, 510, 519, 555, and 705, or a functional variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence set forth in any one of SEQ ID Nos.: 109, 20, 28, 36, 44, 51, 57, 67, 77, 84, 93, 118, 127, 134, 384, 393, 402, 410, 420, 438, 446, 468, 502, 511, 556, and 520, or a functional variant thereof comprising having one, two, or three amino acid substitutions, one or more of which substitutions is optionally a conservative substitution and/or is a substitution to a germline-encoded amino acid.

Embodiment 26. The antibody or antigen-binding fragment of Embodiment 25, wherein: (i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID Nos.: 101, 129, 12, 38, 46, 53, 59, 69, 120, 73, 79, 86, 95, 22, 111, 30, 378, 386, 395, 404, 412, 416, 422, 426, 431, 440, 451, 458, 463, 470, 475, 480, 485, 491, 497, 532, 537, 539, 542, 545, 547, 550, 504, 513, 524, 527, 702, 707, 712, and 716, wherein sequence variation is optionally limited to one or more framework regions and/or sequence variation comprises one or more substitution to a germline-encoded amino acid; and/or (ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID Nos.: 106, 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 553, 558, 560, 563, 567, 570, 572, 574, 704, and 710, wherein sequence variation is optionally limited to one or more framework regions and/or sequence variation comprises one or more substitution to a germline-encoded amino acid.

Embodiment 27. An antibody or antigen-binding fragment comprising a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are according to the IMGT numbering system, wherein the antibody or antigen-binding fragment binds to a respiratory syncytial virus (RSV) fusion glycoprotein (RSV-F) and a metapneumovirus (MPV) fusion glycoprotein (MPV-F) and wherein: (i) the CDRH1 comprises or consists of the amino acid sequence set forth in any one of SEQ ID Nos.: 13, 23, 31, 39, 54, 60, 74, 80, 87, 112, 121, 130, 380, 387, 396, 417, 441, 452, 459, 471, 481, 492, 498, 505, 514, 528, and 533, or a functional variant thereof comprising one, two, or three acid substitutions, one or more of which substitutions is optionally a conservative substitution and/or is a substitution to a germline-encoded amino acid; (ii) the CDRH2 comprises or consists of the amino acid sequence set forth in any one of SEQ ID Nos.: 14, 24, 32, 40, 47, 61, 81, 88, 96, 113, 122, 388, 397, 405, 442, 453, 464, 506, 515, 534, 540, 543, and 548, or a functional variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence set forth in any one of SEQ ID Nos.: 15, 25, 33, 48, 62, 89, 114, 123, 131, 389, 398, 406, 427, 432, 476, 493, 499, 507, 516, 535, and 551, or a functional variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence set forth in any one of SEQ ID NOs.: 18, 65, 92, 99, 126, 383, 401, 437, 445, 456, 488, 554, 561, 564, and 568, or a functional variant thereof comprising one, two, or three amino acid substitutions, one or more of which substitutions is optionally a conservative substitution and/or is a substitution to a germline-encoded amino acid; (v) the CDRL2 comprises or consists of the amino acid sequence set forth in any one of SEQ ID Nos.: 19, 43, 66, 117, 392, 409, 467, 489, 510, 519, 555, and 705, or a functional variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence set forth in any one of SEQ ID Nos.: 20, 28, 36, 44, 51, 57, 67, 77, 84, 93, 118, 127, 134, 384, 393, 402, 410, 420, 438, 446, 468, 502, 511, 520, and 556, or a functional variant thereof comprising having one, two, or three amino acid substitutions, one or more of which substitutions is optionally a conservative substitution and/or is a substitution to a germline-encoded amino acid.

Embodiment 28. The antibody or antigen-binding fragment of embodiment 27, comprising CDRH1, CDRH2, CDRH3, CDRL1, CDRL2, and CDRL3 amino acid sequences set forth in SEQ ID Nos.: 1) SEQ ID NOs.: 130, 47, 131, 18, 117, and 134; 2) SEQ ID NOs.: 13-15 and 18-20; 3) SEQ ID NOs.: 39, 40, 25, 18, 43, and 44; 4) SEQ ID NOs.: 39, 47, 48, 18, 19, and 51; 5) SEQ ID NOs.: 55, 24, 25, 18, 43, and 57; 6) SEQ ID NOs.: 60-62 and 65-67; 7) SEQ ID NOs.: 39, 40, 25, 18, 43, and 44; 8) SEQ ID NOs.: 121-123, 126, 117, and 127; 9) SEQ ID NOs.: 74, 24, 48, 18, 19, and 77; 10) SEQ ID NOs.: 80, 81, 25, 18, 19, and 84; 11) SEQ ID NOs.: 87-89, 92, 19, and 93; 12) SEQ ID NOs.: 39, 96, 48, 99, 19, and 44; 13) SEQ ID NOs.: 23-25, 18, 19, and 28; 14) SEQ ID NOs.: 112-114, 18, 117, and 118; 15) SEQ ID NOs.: 31-33, 18, 19, and 36; 16) SEQ ID NOs.: 380, 81, 25, 383, 19, and 384; 17) SEQ ID NOs.: 387-389, 18, 392, and 393; 18) SEQ ID NOs.: 396-398, 401, 19, and 402; 19) SEQ ID NOs.: 80, 405, 406, 18, 409, and 410; 20) SEQ ID NOs.: 87-89, 401, 19, and 402; 21) SEQ ID NOs.: 417, 24, 48, 18, 19, and 420; 22) SEQ ID NOs.: 396-398, 401, 19, and 402; 23) SEQ ID NOs.: 39, 40, 427, 401, 19, and 28; 24) SEQ ID NOs.: 130, 47, 432, 18, 117, and 134; 25) SEQ ID NOs.: 130, 47, 432, 437, 43, and 438; 26) SEQ ID NOs.: 441, 442, 48, 445, 19, and 446; 27) SEQ ID NOs.: 130, 47, 432, 18, 117, and 134; 28) SEQ ID NOs.: 452, 453, 48, 456, 19, and 384; 29) SEQ ID NOs.: 459, 24, 25, 18, 19, and 402; 30) SEQ ID NOs.: 87, 464, 89, 401, 467, and 468; 31) SEQ ID NOs.: 471, 81, 25, 445, 19, and 384; 32) SEQ ID NOs.: 471, 40, 476, 18, 19, and 51; 33) SEQ ID NOs.: 481, 88, 48, 18, 19, and 51; 34) SEQ ID NOs.: 87, 88, 25, 488, 489, and 402; 35) SEQ ID NOs.: 492, 47, 493, 18, 19, and 384; 36) SEQ ID NOs.: 498, 47, 499, 18, 467, and 502; 37) SEQ ID NOs.: 505, 506, 507, 18, 510, and 511; 38) SEQ ID NOs.: 514, 515, 516, 18, 519, and 520; 39) SEQ ID NOs.: 121-123, 126, 117, and 127; 40) SEQ ID NOs.: 514-516, 18, 519, and 520; 41) SEQ ID NOs.: 528, 88, 25, 401, 19, and 402; 42) SEQ ID NOs.: 533-535 and 554-556; 43) SEQ ID NOs.: 533-535, 561, 555, and 556; 44) SEQ ID NOs.: 533-535, 564, 555, and 556; 45) SEQ ID NOs.: 533-535, 568, 555, and 556; 46) SEQ ID NOs.: 533, 540, 535 and 554-556; 47) SEQ ID NOs.: 533, 540, 535, 561, 555, and 556; 48) SEQ ID NOs.: 533, 540, 535, 564, 555, and 556; 49) SEQ ID NOs.: 533, 540, 535, 568, 555, 556; 50) SEQ ID NOs.: 533, 543, 535, and 554-556; 51) SEQ ID NOs.: 533, 543, 535, 561, 555, and 556; 52) SEQ ID NOs.: 533, 543, 535, 564, 555, and 556; 53) SEQ ID NOs.: 533, 543, 535, 568, 555, and 556; 54) SEQ ID NOs.: 533, 548, 535, and 554-556; 55) SEQ ID NOs.: 533, 548, 535, 561, 555, and 556; 56) SEQ ID NOs.: 533, 548, 535, 564, 555, and 556; 57) SEQ ID NOs.: 533, 548, 535, 564, 555, and 556; 58) SEQ ID NOs.: 533, 548, 535, 568, 555, and 556; 59) SEQ ID NOs.: 121-123, 18, 705, and 127; or 60) 112-114, 18, 705, and 118, respectively.

Embodiment 29. The antibody or antigen-binding fragment of any one of embodiments 25-28, wherein: (i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID Nos.: 129, 12, 38, 46, 53, 59, 69, 120, 73, 79, 86, 95, 22, 111, 30, 378, 386, 395, 404, 412, 416, 422, 426, 431, 440, 451, 458, 463, 470, 475, 480, 485, 491, 497, 504, 513, 524, 527, 532, 537, 539, 542, 545, 547, 550, 702, 707, 712, and 716, wherein sequence variation is optionally limited to one or more framework regions and/or sequence variation comprises one or more substitution to a germline-encoded amino acid; and/or (ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID Nos.: 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 704, 553, 558, 560, 563, 567, 570, 572, 574, 704, and 710, wherein sequence variation is optionally limited to one or more framework regions and/or sequence variation comprises one or more substitution to a germline-encoded amino acid.

Embodiment 30. The antibody or antigen-binding fragment of any one of embodiments 25-29, wherein the VH and the VL comprise or consist of the amino acid sequences set forth in SEQ ID Nos.: 1) SEQ ID NOs.: 129 and 133; 2) SEQ ID NOs.: 12 and 17; 3) SEQ ID NOs.: 38 and 42; 4) SEQ ID NOs.: 46 and 50; 5) SEQ ID NOs.: 53 and 56; 6) SEQ ID NOs.: 59 and 64; 7) SEQ ID NOs.: 69 and 71; 8) SEQ ID NOs.: 120 and 125; 9) SEQ ID NOs.: 73 and 76; 10) SEQ ID NOs.: 79 and 83; 11) SEQ ID NOs.: 86 and 91; 12) SEQ ID NOs.: 95 and 98; 13) SEQ ID NOs.: 22 and 27; 14) SEQ ID NOs.: 111 and 116; 15) SEQ ID NOs.: 30 and 35; 16) SEQ ID NOs.: 378 and 382; 17) SEQ ID NOs.: 386 and 391; 18) SEQ ID NOs.: 395 and 400; 19) SEQ ID NOs.: 404 and 408; 20) SEQ ID NOs.: 412 and 414; 21) SEQ ID NOs.: 416 and 419; 22) SEQ ID NOs.: 422 and 424; 23) SEQ ID NOs.: 426 and 429; 24) SEQ ID NOs.: 431 and 434; 25) SEQ ID NOs.: 431 and 436; 26) SEQ ID NOs.: 440 and 444; 27) SEQ ID NOs.: 431 and 449; 28) SEQ ID NOs.: 451 and 455; 29) SEQ ID NOs.: 458 and 461; 30) SEQ ID NOs.: 463 and 466; 31) SEQ ID NOs.: 470 and 473; 32) SEQ ID NOs.: 475 and 478; 33) SEQ ID NOs.: 480 and 483; 34) SEQ ID NOs.: 485 and 487; 35) SEQ ID NOs.: 491 and 495; 36) SEQ ID NOs.: 497 and 501; 37) SEQ ID NOs.: 504 and 509; 38) SEQ ID NOs.: 513 and 518; 39) SEQ ID NOs.: 120 and 522; 40) SEQ ID NOs.: 524 and 518; 41) SEQ ID NOs.: 527 and 530; 42) SEQ ID Nos.: 532 and 553; 43) SEQ ID Nos.: 532 and 558; 44) SEQ ID NOs.: 532 and 577; 45) SEQ ID NOs.: 532 and 563; 46) SEQ ID NOs.: 532 and 567; 47) SEQ ID NOs.: 532 and 570; 48) SEQ ID NOs.: 532 and 572; 49) SEQ ID NOs.: 532 and 574; 50) SEQ ID NOs.: 537 and 553; 51) SEQ ID NOs.: 537 and 558; 52) SEQ ID NOs.: 537 and 577; 53) SEQ ID NOs.: 537 and 563; 54) SEQ ID NOs.: 537 and 567; 55) SEQ ID NOs.: 537 and 570; 56) SEQ ID NOs.: 537 and 572; 57) SEQ ID NOs.: 537 and 574; 58) SEQ ID NOs.: 539 and 553; 59) SEQ ID NOs.: 539 and 558; 60) SEQ ID NOs.: 539 and 577; 61) SEQ ID NOs.: 539 and 563; 62) SEQ ID NOs.: 539 and 567; 63) SEQ ID NOs.: 539 and 570; 64) SEQ ID NOs.: 539 and 572; 65) SEQ ID NOs.: 539 and 574; 66) SEQ ID NOs.: 542 and 553; 67) SEQ ID NOs.: 542 and 558; 68) SEQ ID NOs.: 542 and 577; 69) SEQ ID NOs.: 542 and 563; 70) SEQ ID NOs.: 542 and 567; 71) SEQ ID NOs.: 542 and 570; 72) SEQ ID NOs.: 542 and 572; 73) SEQ ID NOs.: 542 and 574; 74) SEQ ID NOs.: 545 and 553; 75) SEQ ID NOs.: 545 and 558; 76) SEQ ID NOs.: 545 and 577; 77) SEQ ID NOs.: 545 and 563; 78) SEQ ID NOs.: 545 and 567; 79) SEQ ID NOs.: 545 and 570; 80) SEQ ID NOs.: 545 and 572; 81) SEQ ID NOs.: 545 and 574; 82) SEQ ID NOs.: 547 and 553; 83) SEQ ID NOs.: 547 and 558; 84) SEQ ID NOs.: 547 and 577; 85) SEQ ID NOs.: 547 and 563; 86) SEQ ID NOs.: 547 and 567; 87) SEQ ID NOs.: 547 and 570; 88) SEQ ID NOs.: 547 and 572; 89) SEQ ID NOs.: 547 and 574; 90) SEQ ID NOs.: 550 and 553; 91) SEQ ID NOs.: 702 and 704; 92) SEQ ID NOs.: 707 and 708, 93) SEQ ID NOs.: 707 and 709; 94) SEQ ID NOs.: 712 and 76, and 95) SEQ ID NOs.: 716 and 64, respectively.

Embodiment 31. The antibody or antigen-binding fragment of any one of embodiments 25-30, wherein the VH and the VL comprise or consist of amino acid sequences having at least 90%, at least 91%, at least 92%, at least 93%, at least 94%, at least 95%, at least 96%, at least 97%, at least 98%, or at least 99% identity to the amino acid sequences set forth in: SEQ ID NOs.: 702 and 704, respectively, wherein sequence variation is optionally limited to one or more framework regions and/or sequence variation comprises one or more substitution to a germline-encoded amino acid.

Embodiment 32. The antibody or antigen-binding fragment of any one of embodiments 25-31, wherein the antibody or antigen-binding fragment comprises the CDRH1, CDRH2, CDRH3, CDRL1, CDRL2, and CDRL3 amino acid sequences of the VH and VL amino acid sequences set forth in (i) SEQ ID NOs.: 702 and 704, respectively.

Embodiment 33. The antibody or antigen-binding fragment of any one of embodiments 25-32, wherein (i) the CDRH1 comprises or consists of the amino acid sequence set forth in SEQ ID NO.: 121, or a functional variant thereof comprising one, two, or three acid substitutions, one or more of which substitutions is optionally a conservative substitution and/or is a substitution to a germline-encoded amino acid; (ii) the CDRH2 comprises or consists of the amino acid sequence set forth in SEQ ID NO.: 122, or a functional variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence set forth in SEQ ID NO.: 123, or a functional variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence set forth in SEQ ID NO.: 18, or a functional variant thereof comprising one, two, or three amino acid substitutions, one or more of which substitutions is optionally a conservative substitution and/or is a substitution to a germline-encoded amino acid; (v) the CDRL2 comprises or consists of the amino acid sequence set forth in SEQ ID NO.: 705, or a functional variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence set forth in SEQ ID NO.: 127, or a functional variant thereof comprising having one, two, or three amino acid substitutions, one or more of which substitutions is optionally a conservative substitution and/or is a substitution to a germline-encoded amino acid.

Embodiment 34. The antibody or antigen-binding fragment of any one of embodiments 25-33, wherein the MPV-F comprises a D280N mutation (MPV-F N280).

Embodiment 35. The antibody or antigen-binding fragment of any one of claims 1-34, wherein the antibody or antigen-binding fragment is a IgG, IgA, IgM, IgE, or IgD isotype.

Embodiment 36. The antibody or antigen-binding fragment of any one of embodiments 1-35, wherein the antibody or antigen-binding fragment is an IgG isotype selected from IgG1, IgG2, IgG3, and IgG4.

Embodiment 37. The antibody or antigen-binding fragment of any one of embodiments 1-36, wherein the antibody, or the antigen-binding fragment, comprises a human antibody, a monoclonal antibody, a purified antibody, a single chain antibody, a Fab, a Fab′, a F(ab′)2, or Fv.

Embodiment 38. The antibody or antigen-binding fragment of any one of embodiments 1-37, wherein the antibody or antigen-binding fragment is a multi-specific antibody or antigen-binding fragment, optionally a bispecific antibody or antigen-binding fragment.

Embodiment 39. The antibody or antigen-binding fragment of embodiment 38, comprising: (i) a first VH and a first VL; and (ii) a second VH and a second VL, wherein the first VH and the first VL together form a first antigen-binding site, and wherein the second VH and the second VL together form a second antigen-binding site; wherein the first VH and first VL, respectively, comprise: i) an amino acid sequence having at least 85% identity to the amino acid sequence set forth in any one of SEQ ID NOs.: 2, 136, 146, 159, 169, 175, 181, 189, 196, 202, 210, 215, 225, 233, 243, 250, 254, 261, 271, 277, 284, 293, 300, 309, 316, 321, 327, 332, 342, 347, 352, 357, 362, and 369; and/or an amino acid sequence having at least 85% identity to the amino acid sequence set forth in any one of SEQ ID NOs.: 7, 141, 150, 155, 164, 172, 178, 185, 192, 199, 205, 212, 220, 229, 238, 247, 252, 257, 266, 274, 282, 288, 296, 305, 312, 319, 324, 330, 337, 345, 349, 355, 360, 367, and 374; ii) an amino acid sequence having at least 85% identity to the amino acid sequence set forth in SEQ ID NO.: 101; and/or an amino acid sequence having at least 85% identity to the amino acid sequence set forth in SEQ ID NO.: 106; iii) an amino acid sequence having at least 85% identity to the amino acid sequence set forth in any one of SEQ ID NOs.: 129, 12, 38, 46, 53, 59, 69, 120, 73, 79, 86, 95, 22, 111, 30, 378, 386, 395, 404, 412, 416, 422, 426, 431, 440, 451, 458, 463, 470, 475, 480, 485, 491, 497, 532, 537, 539, 542, 545, 547, 550, 504, 513, 524, 527, 702, 707, 712, and 716; and/or an amino acid sequence having at least 85% identity to the amino acid sequence set forth in any one of SEQ ID NOs.: 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 553, 558, 560, 563, 567, 570, 572, 574, 704, and 710; iv) an amino acid sequence having at least 85% identity to the amino acid sequence set forth in any one of SEQ ID NOs.: 532, 537, 539, 542, 545, 547, and 550; and/or an amino acid sequence having at least 85% identity to the amino acid sequence set forth in any one of SEQ ID NOs.: 553, 558, 560, 563, 567, 570, 572, and 574; and/or v) an amino acid sequence having at least 85% identity to the amino acid sequence set forth in any one of SEQ ID NOs.: 2, 136, 146, 159, 169, 175, 181, 189, 196, 202, 210, 215, 225, 233, 243, 250, 254, 261, 271, 277, 284, 293, 300, 309, 316, 321, 327, 332, 342, 347, 352, 357, 362, 369, 369, 727, 737, 746, 755, 765, 775, 784, 794, 804, 813, 817, 820, 823, 826, 828, 831, 834, 837, 840, 843, 883, 886, 889, 893, 896, 899, 901, 903, 101, 129, 12, 38, 46, 53, 59, 69, 120, 73, 79, 86, 95, 22, 111, 30, 378, 386, 395, 404, 412, 416, 422, 426, 431, 440, 451, 458, 463, 470, 475, 480, 485, 491, 497, 504, 513, 524, 527, 702, 532, 537, 539, 542, 545, 547, 550, 707, 712, and 716; and/or an amino sequences having at least 85% identity to the amino acid sequence set forth in any one of SEQ ID NOs.: 7, 141, 150, 155, 164, 172, 178, 185, 192, 199, 205, 212, 220, 229, 238, 247, 252, 257, 266, 274, 282, 288, 296, 305, 312, 319, 324, 330, 337, 345, 349, 355, 360, 367, 374, 732, 742, 751, 760, 770, 780, 789, 799, 808, 847, 851, 855, 858, 860, 862, 865, 868, 870, 873, 875, 877, 879, 881, 106, 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 704, 553, 558, 560, 563, 567, 570, 572, 574, and 710, and wherein the second VH and second VL are not both the same as the first VH and first VH, and, respectively, comprise: i) an amino acid sequence having at least 85% identity to the amino acid sequence set forth in any one of SEQ ID NOs.: 2, 136, 146, 159, 169, 175, 181, 189, 196, 202, 210, 215, 225, 233, 243, 250, 254, 261, 271, 277, 284, 293, 300, 309, 316, 321, 327, 332, 342, 347, 352, 357, 362, 369, 727, 737, 746, 755, 765, 775, 784, 794, 804, 813, 817, 820, 823, 826, 828, 831, 834, 837, 840, 843, 883, 886, 889, 893, 896, 899, 901, and 903; and/or an amino acid sequence having at least 85% identity to the amino acid sequence set forth in any one of SEQ ID NOs.: 7, 141, 150, 155, 164, 172, 178, 185, 192, 199, 205, 212, 220, 229, 238, 247, 252, 257, 266, 274, 282, 288, 296, 305, 312, 319, 324, 330, 337, 345, 349, 355, 360, 367, 374 732, 742, 751, 760, 770, 780, 789, 799, 808, 847, 851, 855, 858, 860, 862, 865, 868, 870, 873, 875, 877, 879, and 881; ii) an amino acid sequence having at least 85% identity to the amino acid sequence set forth in SEQ ID NO.: 101; and/or an amino acid sequence having at least 85% identity to the amino acid sequence set forth in SEQ ID NO.: 106; iii) an amino acid sequence having at least 85% identity to the amino acid sequence set forth in any one of SEQ ID NOs.: 129, 12, 38, 46, 53, 59, 69, 120, 73, 79, 86, 95, 22, 111, 30, 378, 386, 395, 404, 412, 416, 422, 426, 431, 440, 451, 458, 463, 470, 475, 480, 485, 491, 497, 532, 537, 539, 542, 545, 547, 550, 504, 513, 524, 527, 702, 707, 712, and 716; and/or an amino acid sequence having at least 85% identity to the amino acid sequence set forth in any one of SEQ ID NOs.: 7, 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 553, 558, 560, 563, 567, 570, 572, 574, 704, and 710; iv) an amino acid sequence having at least 85% identity to the amino acid sequence set forth in any one of SEQ ID NOs.: 532, 537, 539, 542, 545, 547, and 550; and/or an amino acid sequence having at least 85% identity to the amino acid sequence set forth in any one of SEQ ID NOs.: 553, 558, 560, 563, 567, 570, 572, and 574; v) an amino acid sequence having at least 85% identity to the amino acid sequence set forth in any one of SEQ ID NOs.: 576, 586, 591, 600, 604, 613, and 617; and/or an amino acid sequence having at least 85% identity to the amino acid sequence set forth in any one of SEQ ID NOs.: 581, 588, 596, 602, 609, 615, and 622; vi) an amino acid sequence having at least 85% identity to the amino acid sequence set forth in any one of SEQ ID NOs.: 627, 636, 641, 648, 657, and 659; and/or an amino acid sequence having at least 85% identity to the amino acid sequence set forth in any one of SEQ ID NOs.: 632, 638, 645, 653, and 662; and/or vii) an amino acid sequence having at least 85% identity to the amino acid sequence set forth in any one of SEQ ID NOs.: 2, 136, 146, 159, 169, 175, 181, 189, 196, 202, 210, 215, 225, 233, 243, 250, 254, 261, 271, 277, 284, 293, 300, 309, 316, 321, 327, 332, 342, 347, 352, 357, 362, 369, 727, 737, 746, 755, 765, 775, 784, 794, 804, 813, 817, 820, 823, 826, 828, 831, 834, 837, 840, 843, 883, 886, 889, 893, 896, 899, 901, 903, 101, 129, 12, 38, 46, 53, 59, 69, 120, 73, 79, 86, 95, 22, 111, 30, 378, 386, 395, 404, 412, 416, 422, 426, 431, 440, 451, 458, 463, 470, 475, 480, 485, 491, 497, 504, 513, 524, 527, 702, 532, 537, 539, 542, 545, 547, 550, 576, 586, 591, 600, 604, 613, 617, 627, 636, 641, 648, 657, 659, 707, 712, and 716; and/or an amino sequences having at least 85% identity to the amino acid sequence set forth in any one of SEQ ID NOs.: 7, 141, 150, 155, 164, 172, 178, 185, 192, 199, 205, 212, 220, 229, 238, 247, 252, 257, 266, 274, 282, 288, 296, 305, 312, 319, 324, 330, 337, 345, 349, 355, 360, 367, 374, 732, 742, 751, 760, 770, 780, 789, 799, 808, 847, 851, 855, 858, 860, 862, 865, 868, 870, 873, 875, 877, 879, 881, 106, 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 704, 553, 558, 560, 563, 567, 570, 572, 574, 581, 588, 596, 602, 609, 615, 622, 632, 638, 645, 653, 662, and 710.

Embodiment 40. The antibody or antigen-binding fragment of embodiment 38, comprising: (i) a first VH and a first VL; and (ii) a second VH and a second VL, wherein the first VH and the first VL together form a first antigen-binding site, and wherein the second VH and the second VL together form a second antigen-binding site; wherein the first VH and first VL, respectively, comprise the VH and a VL set forth in SEQ ID NOs.: 136 and 851; 814 and 141; 233 and 858; 837 and 858; 357 and 847; and 899 and 360, respectively.

Embodiment 41. The antibody or antigen-binding fragment of embodiment 38, comprising: (i) a first VH and a first VL; and (ii) a second VH and a second VL, wherein the first VH and the first VL together form a first antigen-binding site, and wherein the second VH and the second VL together form a second antigen-binding site; wherein the first VH and first VL, respectively, comprise the VH and VL as set forth in SEQ ID NOs.: 702 and 704.

Embodiment 42. The antibody or antigen-binding fragment of embodiment 38, comprising: (i) a first VH and a first VL; and (ii) a second VH and a second VL, wherein the first VH and the first VL together form a first antigen-binding site, and wherein the second VH and the second VL together form a second antigen-binding site; wherein the first VH and VL comprise a VH and a VL set forth in SEQ ID NOs.: 136 and 851; 814 and 141; 233 and 858; 837 and 858; 357 and 847; and 899 and 360, respectively; and the second VH and VL comprise a VH and VL as set forth in SEQ ID NOs.: 702 and 704.

Embodiment 43. The antibody or antigen-binding fragment of any one of embodiments 1-42, wherein the antibody or antigen-binding fragment comprises a Fc polypeptide or a fragment thereof, wherein, optionally the Fc polypeptide may comprise i) a heavy chain (HC) and a light chain (LC), or ii) two heavy chains (HCs) and two light chains (LCs).

Embodiment 44. The antibody or antigen-binding fragment of embodiment 43, wherein the Fc polypeptide or fragment comprises: (i) a mutation that enhances binding to a FcRn as compared to a reference Fc polypeptide that does not comprise the mutation; (ii) a mutation that enhances binding to a FcγR as compared to a reference Fc polypeptide that does not comprise the mutation; (iii) a mutation that enhances binding to human FcγRIIa and/or decreases binding to a human FcγRIIb as compared to a reference Fc polypeptide that does not comprise the mutation; and/or (iv) a mutation that enhances binding to a human C1q compared to a reference Fc polypeptide that does not comprise the mutation.

Embodiment 45. The antibody or antigen-binding fragment of embodiment 44, wherein the mutation that increases binding affinity to a human FcRn comprises: M428L; N434S; N434H; N434A; N434S; M252Y; S254T; T256E; T250Q; P257I; Q311I; D376V; T307A; E380A; or any combination thereof.

Embodiment 46. The antibody or antigen-binding fragment of embodiment 44 or embodiment 45, wherein the mutation that increases binding affinity to a human FcRn comprises: (i) M428L/N434S; (ii) M252Y/S254T/T256E; (iii) T250Q/M428L; (iv) P257I/Q311I; (v) P257I/N434H; (vi) D376V/N434H; (vii) T307A/E380A/N434A; or (viii) any combination of (i)-(vii).

Embodiment 47. The antibody or antigen-binding fragment of any one of embodiments 44-46, wherein the mutation that enhances binding to a FcγR comprises: (i) S239D; 1332E; A330L; G236A; or any combination thereof, (ii) S239D/1332E; (iii) S239D/A330L/1332E; (iv) G236A/S239D/1332E; or (v) G236A/A330L/1332E, wherein the Fc polypeptide or fragment optionally comprises Ser at position 239.

Embodiment 48. The antibody or antigen-binding fragment of any one of embodiments 44-47, wherein the Fc polypeptide comprises the substitution mutations M428L/N434S, M428L/N434A, G236A/A330L/I332E/M428L/N434S, or G236A/A330L/I332E/M428L/N434A, wherein, optionally, the antibody or antigen-binding fragment is an IgGI isotype, and comprises or consists of a Fc polypeptide or fragment thereof that comprises or consists of amino acid sequences having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NOs.: 664-700, optionally other than naturally occurring variants thereof, or that comprises or consists of, the amino acid sequences set forth in SEQ ID NOs.: 670-700.

Embodiment 49. The antibody or antigen-binding fragment of any one of embodiments 44-48, wherein the Fc polypeptide comprises or consists of a Fc polypeptide or fragment thereof that comprises or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs.: 679-684 and 688-690, optionally other than naturally occurring variants thereof, or that comprises or consists of an amino acid sequence set forth in any one of SEQ ID NOs.: 679-684 and 688-690.

Embodiment 50. The antibody or antigen-binding fragment of any one of embodiments 44-49, wherein the antibody comprises
i) a heavy chain (HC) that comprises or consists of a polypeptide or fragment thereof that comprises or consists of the amino acid sequence set forth in SEQ ID NO.: 723, and a light chain (LC) that comprises or consists of a polypeptide or fragment thereof that comprises or consists of the amino acid sequence set forth in SEQ ID NO.: 725; or
ii) two heavy chains (HCs) that both comprise or consist of a polypeptide or fragment thereof that comprises or consists of the amino acid sequence set forth in SEQ ID NO.: 723, and two light chains (LCs) that both comprise or consist of a polypeptide or fragment thereof that comprises or consists of the amino acid sequence set forth in SEQ ID NO.: 725

Embodiment 51. The antibody or antigen-binding fragment of any one of embodiments 44-50, which comprises a mutation that alters glycosylation, wherein the mutation that alters glycosylation comprises N297A, N297Q, or N297G, and/or which is aglycosylated and/or afucosylated.

Embodiment 52. The antibody or antigen-binding fragment of any one of embodiments 1-51, which is human, humanized, or chimeric.

Embodiment 53. The antibody or antigen-binding fragment of any one of embodiments 1-52, wherein the antibody or antigen-binding fragment thereof binds to a) both a RSV A and RSV B strain; b) both a MPV A and MPV B strain; c) any combinations of an RSV A, RSV B, MPV A, and MPV B strain.

Embodiment 54. The antibody or antigen-binding fragment of any one of embodiments 1-53, wherein the RSV-F comprises or consists of a stabilized trimer of a pre-fusion conformation of component proteins, optionally DS-Cav1.

Embodiment 55. The antibody or antigen-binding fragment of any one of embodiments 1-54, which is activates a human FcγRIIa.

Embodiment 56. The antibody or antigen-binding fragment of embodiment 55, wherein activation is as determined using a host cell (optionally, a Jurkat cell) comprising: (i) the human FcγRIIIa (optionally, a H131 allele); and (ii) a NFAT expression control sequence operably linked to a sequence encoding a reporter, such as a luciferase reporter, following incubation of the antibody or antigen-binding fragment with a target cell (optionally, a Expi293 cell) transfected with a fusion glycoprotein from RSV and/or MPV.

Embodiment 57. The antibody or antigen-binding fragment of any one of embodiments 1-56, wherein the antibody activates a human FcγRIIIa.

Embodiment 58. The antibody or antigen-binding fragment of embodiment 57, wherein activation is as determined using a host cell (optionally, a Jurkat cell) comprising: (i) the human FcγRIIIa (optionally, a F158 allele); and (ii) a NFAT expression control sequence operably linked to a sequence encoding a reporter, such as a luciferase reporter, following incubation of the antibody or antigen-binding fragment with a target cell (optionally, a Expi293 cell) transfected with a fusion glycoprotein from RSV and/or MPV.

Embodiment 59. The antibody or antigen-binding fragment of any one of embodiments 1-58, wherein the antibody neutralizes infection by a RSV and/or MPV.

Embodiment 60. The antibody or antigen-binding fragment of any one of embodiments 1-59, wherein the antibody or antigen-binding fragment treats and/or prevents (i) a RSV infection and/or (ii) a MPV infection in a subject.

Embodiment 61. An isolated polynucleotide encoding the antibody or antigen-binding fragment of any one of embodiments 1-60, or encoding a VH, a heavy chain, a VL, a light chain and/or one or more CDR of the antibody or the antigen-binding fragment.

Embodiment 62. The polynucleotide of embodiment 61, wherein the polynucleotide comprises deoxyribonucleic acid (DNA) or ribonucleic acid (RNA), wherein the RNA optionally comprises messenger RNA (mRNA).

Embodiment 63. The polynucleotide of embodiment 61 or 62, wherein the polynucleotide is codon-optimized for expression in a host cell.

Embodiment 64. The polynucleotide of any one of embodiments 61-63, wherein the antibody or antigen-binding fragment binds RSV-F, wherein the polynucleotide comprises a polynucleotide having at least 50% (e.g., 50%, 55%, 60%, 65%, 70%, 75%, 80%, 85%, 90%, 91%, 92%, 94%, 94%, 95%, 96%, 97%, 98%, 99%, or more) identity to the VH-encoding polynucleotide sequence set forth in any one or more of SEQ ID NOs.: 1, 135, 145, 158, 168, 174, 180, 188, 195, 201, 209, 214, 232, 249, 253, 260, 270, 276, 283, 292, 299, 308, 315, 320, 326, 331, 341, 346, 351, 356, 361, 368, 726, 736, 745, 754, 764, 774, 783, 793, 803, 811, 812, 816, 819, 822, 825, 827, 829, 830, 833, 836, 839, 842, 882, 885, 888, 891, 892, 895, 898, 900, 902, 128, 11, 37, 45, 52, 58, 68, 119, 72, 78, 85, 94, 21, 110, 29, 377, 385, 394, 403, 411, 415, 421, 425, 430, 439, 447, 450, 457, 462, 469, 474, 479, 484, 490, 496, 503, 512, 523, 526, 701, 706, 711, and 715, and/or the VL-encoding polynucleotide sequence set forth in any one or more of SEQ ID NOs.: 6, 140, 149, 154, 163, 171, 177, 184, 191, 198, 204, 211, 219, 228, 237, 246, 251, 256, 265, 273, 281, 287, 295, 304, 311, 318, 323, 329, 336, 344, 348, 354, 359, 366, 373, 731, 741, 750, 759, 769, 779, 788, 798, 807, 845, 846, 849, 850, 853, 854, 857, 859, 861, 863, 864, 867, 869, 872, 874, 876, 878, 880, 132, 16, 41, 49, 55, 63, 70, 124, 75, 82, 90, 97, 26, 115, 34, 381, 390, 399, 407, 413, 418, 423, 428, 433, 435, 443, 448, 454, 460, 465, 472, 477, 482, 486, 494, 500, 508, 517, 521, 525, 529, 703, 708, 709, 713, 714, 717.

Embodiment 65. The polynucleotide of any one of embodiments 61-64, wherein the antibody or antigen-binding fragment binds MPV-F, wherein the polynucleotide comprises a polynucleotide having at least 50% (e.g., 50%, 55%, 60%, 65%, 70%, 75%, 80%, 85%, 90%, 91%, 92%, 94%, 94%, 95%, 96%, 97%, 98%, 99%, or more) identity to the VH-encoding polynucleotide sequence set forth in any one or more of SEQ ID NOs.: 100, 128, 11, 37, 45, 52, 58, 68, 119, 72, 78, 85, 94, 21, 110, 29, 377, 385, 394, 403, 411, 415, 421, 425, 430, 439, 447, 450, 457, 462, 469, 474, 479, 484, 490, 496, 503, 512, 523, 526, 701, 706, 711, and 715, and/or the VL-encoding polynucleotide sequence set forth in any one or more of SEQ ID NOs.: 105, 132, 16, 41, 49, 55, 63, 70, 124, 75, 82, 90, 97, 26, 115, 34, 381, 390, 399, 407, 413, 418, 423, 428, 433, 435, 443, 448, 454, 460, 465, 472, 477, 482, 486, 494, 500, 508, 517, 521, 525, 529, 703, 708, 709, 713, 714, 717.

Embodiment 66. The polynucleotide of any one of embodiments 61-64, wherein the antibody or antigen-binding fragment binds RSV-F and MPV-F, wherein the polynucleotide comprises a polynucleotide having at least 50% (e.g., 50%, 55%, 60%, 65%, 70%, 75%, 80%, 85%, 90%, 91%, 92%, 94%, 94%, 95%, 96%, 97%, 98%, 99%, or more) identity to the VH-encoding polynucleotide sequence set forth in any one or more of SEQ ID NOs.: 128, 11, 37, 45, 52, 58, 68, 119, 72, 78, 85, 94, 21, 110, 29, 377, 385, 394, 403, 411, 415, 421, 425, 430, 439, 447, 450, 457, 462, 469, 474, 479, 484, 490, 496, 503, 512, 523, 526, 701, 706, 711, and 715, and/or the VL-encoding polynucleotide sequence set forth in any one or more of SEQ ID NOs.: 132, 16, 41, 49, 55, 63, 70, 124, 75, 82, 90, 97, 26, 115, 34, 381, 390, 399, 407, 413, 418, 423, 428, 433, 435, 443, 448, 454, 460, 465, 472, 477, 482, 486, 494, 500, 508, 517, 521, 525, 529, 703, 708, 709, 713, 714, 717.

Embodiment 67. The polynucleotide of any one of embodiments 61-64, wherein the antibody or antigen-binding fragment binds RSV-F and/or MPV-F, wherein the polynucleotide comprises a polynucleotide having at least 50% (e.g., 50%, 55%, 60%, 65%, 70%, 7%, 80%, 85, 90, 91%, 92%, 94%, 94%, 95%, 96%, 97%, 98%, 99%, or more) identity to the VH-encoding polynucleotide sequence set forth in any one or more of SEQ ID NOs.: 701, 135, 816, 232, 836, 356, 898, and/or the VL-encoding polynucleotide sequence set forth in any one or more of SEQ ID NOs.: 703, 850, 140, 857, 846, 359.

Embodiment 68. A recombinant vector comprising the polynucleotide of any one of embodiments 61-67.

Embodiment 69. A host cell comprising the polynucleotide of any one of embodiments 61-67 and/or the vector of embodiment 68, wherein the polynucleotide is heterologous to the host cell and wherein the host cell expresses the encoded antibody or antigen-binding fragment.

Embodiment 70. An isolated human B cell comprising the polynucleotide of any one of embodiments 61-67 and/or the vector of embodiment 68, wherein polynucleotide is heterologous to the human B cell and/or wherein the human B cell is immortalized.

Embodiment 71. A composition comprising: (i) the antibody or antigen-binding fragment of any one of embodiments 1-60; (ii) the polynucleotide of any one of embodiments 61-67; (iii) the recombinant vector of embodiment 68; (iv) the host cell of embodiment 69; and/or (v) the human B cell of embodiment 70, and a pharmaceutically acceptable excipient, carrier, or diluent.

Embodiment 72. The composition of embodiment 71, comprising a first antibody or antigen-binding fragment and a second antibody or antigen-binding fragment, wherein each of the first antibody or antigen-binding fragment and the second antibody or antigen-binding fragment are different and are each according any one of embodiments 1-60, or at least one is according to any one of embodiments 1-60 and at least one is palivizumab, nirsevimab or clesrovimab, or and antibody having a VH and VL of any one of these antibodies.

Embodiment 73. The composition of embodiment 71 or embodiment 72, comprising a first antibody or antigen-binding fragment and a second antibody or antigen-binding fragment, wherein the first antibody or antigen-binding fragment comprises at least a VH and VL or CDRs of the VH and a VL set forth in any one of SEQ ID NOs.: 136 and 851; 814 and 141; 233 and 858; 837 and 858; 357 and 847; and 899 and 360, respectively.

Embodiment 74. The composition of any one of embodiments 71-73, comprising a first antibody or antigen-binding fragment and a second antibody or antigen-binding fragment, wherein the second antibody or antigen-binding fragment comprises at least a VH and VL or CDRs of the VH and VL as set forth in SEQ ID NOs.: 702 and 704.

Embodiment 75. The composition of any one of embodiments 71-74, comprising a first antibody or antigen-binding fragment and a second antibody or antigen-binding fragment, wherein the first antibody or antigen-binding fragment comprises the VH and a VL set forth in any one of SEQ ID NOs.: 136 and 851; 814 and 141; 233 and 858; 837 and 858; 357 and 847; and 899 and 360, respectively; and the second antibody or antigen-binding fragment comprises a second VH and VL or second CDRs of the VH and VL as set forth in SEQ ID NOs.: 702 and 704.

Embodiment 76. A composition comprising the polynucleotide of any one of embodiments 61-67 or the vector of embodiment 68 encapsulated in a carrier molecule, wherein the carrier molecule optionally comprises a lipid, a lipid-derived delivery vehicle, such as a liposome, a solid lipid nanoparticle, an oily suspension, a submicron lipid emulsion, a lipid microbubble, an inverse lipid micelle, a cochlear liposome, a lipid microtubule, a lipid microcylinder, lipid nanoparticle (LNP), or a nanoscale platform.

Embodiment 77. A method of making an antibody or antigen-binding fragment of any one of embodiments 1-60, comprising culturing the host cell of embodiment 69 or the human B cell of embodiment 70 for a time and under conditions sufficient for the host cell or human B cell, respectively, to express the antibody or antigen-binding fragment.

Embodiment 78. The method of embodiment 77, further comprising isolating the antibody or antigen-binding fragment.

Embodiment 79. A method of treating and/or preventing a RSV infection and/or a MPV infection in a subject, the method comprising administering to the subject an effective amount of: (i) the antibody or antigen-binding fragment of any one of embodiments 1-60; (ii) the polynucleotide of any one of embodiments 61-67; (iii) the recombinant vector of embodiment 68; (iv) the host cell of embodiment 69; (v) the human B cell of embodiment 70; and/or (vi) the composition of any one of embodiments 71-76.

Embodiment 80. The method of embodiment 79, wherein the treatment and/or prevention comprises post-exposure prophylaxis.

Embodiment 81. The method of embodiment 78 or 79, wherein the subject has received, is receiving, or will receive an antiviral.

Embodiment 82. The method of embodiment 81, wherein the antiviral comprises a ribavirin.

Embodiment 83. The method of embodiment 79, wherein the subject has received pre-exposure prophylaxis treatment.

Embodiment 84. The method of embodiment 83, wherein the pre-exposure prophylaxis is palivizumab, nirsevimab or clesrovimab, or and antibody having a VH and VL of any one of these antibodies.

Embodiment 85. The method of embodiment 83, wherein the pre-exposure prophylaxis is an anti-RSV vaccine Embodiment 86. The antibody or antigen-binding fragment of any one of embodiments 1-60, the polynucleotide of any one of embodiments 61-67, the recombinant vector of embodiment 68, the host cell of embodiment 69, the human B cell of embodiment 70, and/or the composition of any one of embodiments 71-76, for use in a method of treating or preventing a RSV infection and/or a MPV infection in a subject.

Embodiment 87. The antibody or antigen-binding fragment of any one of embodiments 1-60, the polynucleotide of any one of embodiments 61-67, the recombinant vector of embodiment 68, the host cell of embodiment 69, the human B cell of embodiment 70, and/or the composition of any one of embodiments 71-76, for use in the preparation of a medicament for the treatment or prevention of a RSV infection and/or a MPV infection in a subject.

Embodiment 88. The method of any one of embodiments 79-85 or the antibody or antigen-binding fragment, the polynucleotide, the recombinant vector, the host cell, the human B cell, and/or the composition for use of any one of embodiments 86-87, wherein: a) the RSV comprises both a RSV A and RSV B strain; b) the MPV comprises both a MPV A and MPV B strain; c) the RSV and MPV comprise any combinations of a RSV A, RSV B, MPV A, and MPV B strain.

Embodiment 89. A method for in vitro diagnosis of a RSV infection and/or a MPV infection, the method comprising: (i) contacting a sample from a subject with an antibody or antigen-binding fragment of any one of embodiments 1-60; and (ii) detecting a complex comprising an antigen and the antibody, or comprising an antigen and the antigen-binding fragment.

Embodiment 90. A kit comprising a liquid composition comprising and antibody or antigen-binding fragment of any one of embodiments 1-60, a polynucleotide according to any one of embodiments 61-67, a recombinant vector according to embodiment 68, a host cell of any one of embodiments 69 or 70, or a composition of any one of embodiments 71-76 and instructions for use thereof in treating a RSV and/or MPV infection in a subject.

Embodiment 91. The kit of Embodiment 90, wherein the instructions for use are for the method of any one of Embodiments 79-85 or the use according to any one of Embodiments 86-87.

TABLE 1
SEQUENCES

| | SEQ ID | |
|---|---|---|
| Identifier | NO | Sequence |
| | 1-8 | See Sequence Listing |
| MPK44; MPK202 CDR-L2 (aa) | 9 | LGS |
| | 10-17 | See Sequence Listing |
| MPK10; MPK9; MPK18; | 18 | SSNIGAGYD |
| MPK30-v1; MPK36-v3; | | |
| MPK67; MPK77; MPK77-v1.1; | | |
| MPK86; MPK102; MPK104; | | |
| MPK104-v1.1; MPK104-v1.3; | | |
| MPK108; MPK127; MPK130; | | |
| MPK133; MPK142-v1; MPK145; | | |
| MPK149; MPK190- v1.3; CDR- | | |
| L1 (aa) | | |
| MPK10; MPK30-v1; MPK77; | 19 | RNT |
| MPK77-v1.1; MPK86; MPK92; | | |
| MPK99; MPK102; MPK108; | | |
| MPK126; MPK152; MPK153; | | |
| MPK157; MPK158; MPK162; | | |
| MPK174-v2; MPK196; MPK129; | | |
| MPK132-v1; MPK133; MPK136; | | |
| MPK141; MPK144; MPK146; | | |
| MPK149; MPK151; MPK152; | | |
| MPK153; MPK157; MPK204 | | |
| CDR-L2 (aa) | | |
| | 20-42 | See Sequence Listing |
| MPK18; MPK36-v3; MPK67 | 43 | RNF |
| CDR-L2 (aa) | | |
| | 44-65 | See Sequence Listing |
| MPK51 CDR-L2 (aa) and | 66 | RNN |
| MPK51-v1.1 CDR-L2 (aa) | | |
| | 67-107 | See Sequence Listing |
| MPK15 CDR-L2 (aa) | 108 | DTS |
| | 109-116 | See Sequence Listing |
| MPK104; MPK9; MPK73; | 117 | GNN |
| MPK104-v1.1; MPK142-v1; | | |
| MPK145; MPK190; MPK190- | | |
| v1.1 CDR-L2(aa) | | |
| | 118-120 | See Sequence Listing |
| MPK73 CDR-H1 (aa) and | 121 | GFSISGYG |
| MPK190; MPK190-v1.1; | | |
| MPK190- v1.3 CRD-H1 (aa) | | |
| MPK73 CDR-H2 (aa) and | 122 | ISASSTYI |
| MPK190; MPK190-v1.1; | | |
| MPK190- v1.3 CDR-H2 (aa) | | |
| MPK73 CDR-H3 (aa) and MPK | 123 | ARAPIEPPYFDL |
| 190; MPK190-v1.1; MPK190- | | |
| v1.3 CDR-H3 (aa) | | |
| | 124-126 | See Sequence Listing |
| MPK73; MPK190; MPK190- | 127 | QSYDRRLSGPI |
| v1.1; MPK190- v1.3 CDR-L3 | | |
| (aa) | | |
| | 128-134 | See Sequence Listing |
| MPK65-v2 VH (nt) (VH.1)(non- | 135 | CAGGTCCCGCTGGTGCAGTCTGGAGCAGAGGTGAAA |
| optimized) | | AAGCCCGGGGAGTCTCTGAAGATCTCCTGTAAGGGTT |
| | | CTGGATACAGCTTTATTAATTACTGGATCGGCTGGGTG |
| | | CGCCAGATGCCCGGGAAAGGCCTGGAGTGGATGGGA |
| | | GTCATCTATCCTTTTGACTCTGATACCAAATACAGCCCG |
| | | TCCTTCCAAGGCCACGTCACCATCTCAGCCGACAAGT |
| | | CCAGCAACACCGCCTACGTGCAGTGGAGCAGCCTGAA |
| | | GGCCTCGGACACCGCCATGTATTACTGTGCGAGATTTA |
| | | CCTTTGGTATTGCAGTGGTCACAGATGATGTTAGTGGG |
| | | GAAGGCCAAGCTTTTGAAATCTGGGGCCAAGGGACA |
| | | ATGGTCACCGTCTCTTCAG |
| MPK65-v2 VH (aa) (VH.1) | 136 | QVPLVQSGAEVKKPGESLKISCKGSGYSFINYWIGWVR |
| | | QMPGKGLEWMGVIYPFDSDTKYSPSFQGHVTISADKSS |
| | | INTAYVQWSSLKASDTAMYYCARFTFGIAVVTDDVSGE |
| | | GQAFEIWGQGTMVTVSS |
| MPK65-v2 VH.1; MPK167; | 137 | GYSFINYW |
| MPK169-v2; MPK170; | | |
| MPK171-v1; MPK171-v2; | | |
| MPK175; MPK179-v4; MPK181; | | |
| MPK186; MPK188; MPK189; | | |
| MPK191; MPK195; MPK198; | | |
| MPK201; MPK201 VH.2; | | |
| MPK201 VH.3 CDR-H1 (aa) | | |
| MPK65-v2 VH.1; MPK65 VH.4; | 138 | TYPFDSDT |
| MPK65 VH.5; MPK161-v1; | | |
| MPK161-v2; MPK165; MPK167; | | |
| MPK168; MPK169-v2; MPK170; | | |
| MPK171-v1; MPK171-v2; | | |
| MPK177; MPK178; MPK179-v4; | | |
| MPK181; MPK185; MPK188; | | |
| MPK189; MPK191; MPK193; | | |
| MPK195; MPK198 CDR-H2 (aa) | | |
| MPK65-v2; MPK65 VH.2; | 139 | ARFTFGIAVVTDDVSGEGQAFEI |
| MPK65 VH.3; VH.1 CDR-H3 | | |
| (aa) | | |
| MPK65-v2 VK.1 VL (nt) | 140 | GAAATAGTGATGATGCAGTCTCCAGCCACCCTGTCTGT |
| | | GTCTCCAGGGGAAAGAGCCACCCTCTCCTGCAGGGCC |
| | | AGTCAGAGTGTTGCCGGCAACTTAGCCTGGTACCAGC |
| | | AGAGACCTGGCCAGGCTCCCAGGCTCCTCATTTATGGT |
| | | GCATCCACCAGGGCCACTGGTGTCCCAGCCAGGTTCA |
| | | GTGGCAGTGGGTCTGAGACAGAGTTCACTCTCACCAT |
| | | CAGCAGCCTGCAGTCTGAAGATTTTGCAGTTTATTACT |
| | | GTCACCAGTATAGTAACTGGCCTCAAGCCTTCGGCCAA |
| | | GGGACACGACTGGACATTAAAC |
| MPK65-v2 VK.1 VL (aa) | 141 | EIVMMQSPATLSVSPGERATLSCRASQSVAGNLAWYQQ |
| | | RPGQAPRLLIYGASTRATGVPARFSGSGSETEFTLTISSLQ |
| | | SEDFAVYYCHQYSNWPQAFGQGTRLDIK |
| MPK65-v2 VK.1; MPK65 VK.2; | 142 | QSVAGN |
| MPK161-v2; MPK167; MPK170; | | |
| MPK171-v2; MPK178; MPK189; | | |
| MPK191; MPK193; MPK198; | | |
| MPK201; MPK201 VK.2 CDR- | | |
| L1 (aa) | | |
| MPK65-v2 VK.1; MPK65 VK.2; | 143 | GAS |
| MPK142-v2; MPK201 VK.2; | | |
| MPK203; MPK167; MPK168; | | |
| MPK169-v2; MPK171-v2; | | |
| MPK175; MPK177; MPK181; | | |
| MPK186; MPK189; MPK191; | | |
| MPK193; MPK195; MPK198; | | |
| MPK201 CDR-L2 (aa) | | |
| MPK65-v2 VK.1 CDR-L3 (aa) | 144 | HQYSNWPQA |
| | 145-151 | See Sequence Listing |
| MPK161-v1 CDR-L2 (aa) | 152 | KAS |
| | 153-155 | See Sequence Listing |
| MPK161-v2; MPK165; MPK178; | 156 | AAS |
| MPK179-v4 CDR-L2 (aa) | | |
| | 157-165 | See Sequence Listing |
| MPK163 CDR-L2 (aa) | 166 | NND |
| | 167-199 | See Sequence Listing |
| MPK170 CDR-L2 (aa) | 200 | AGS |
| | 201-206 | See Sequence Listing |
| MPK171-v1; MPK182 CDR-L2 | 207 | NTD |
| (aa) | | |
| | 208-221 | See Sequence Listing |
| MPK173 CDR-L2 (aa) | 222 | KVS |
| | 223-225 | See Sequence Listing |
| MPK175; MPK186; MPK201; | 226 | IYPEDSET |
| MPK201 VH.4 CDR-H2 (aa) | | |
| | 227-230 | See Sequence Listing |
| MPK175; MPK201 CDR-L3 (aa) | 231 | HHYSDWPQA |
| MPK176 VH.1; MPK176 VH (nt) | 232 | CAGGTGCAGCTGGTGCAGTCTGGAGCTGAGGTGAAG |
| | | AAGCCAGGGGCCTCAGTGAGGGTCTCCTGCAAGGCTT |
| | | CAGGTTACACCCTTACCGCCTACGGTATCAGCTGGGTG |
| | | CGACAGGCCCCTGGACAAGGCCTTGAGTGGATGGGGT |
| | | GGATCAGCCCTAAGCATGGGAACACACACTATGCACA |
| | | GAGGTTCCAGGGCAGAGTCACCATGACCACAGACAC |
| | | GTCCACCAGTACAGCCTACATGGAACTGAGGAGCCTG |
| | | AGACCTGACGACACCGCCGTTTATTTCTGTGCGAGAG |
| | | AGGGCGTGGATATAATGGCGGTTCCCTGGTATTTTGAC |
| | | TACTGGGGCCAGGGAACCCTGGTCGCCGTCTCCTCAG |
| MPK176 VH.1; MPK176 VH | 233 | QVQLVQSGAEVKKPGASVRVSCKASGYTLTAYGISWVR |
| (aa) | | QAPGQGLEWMGWISPKHGNTHYAQRFQGRVTMITDTS |
| | | TSTAYMELRSLRPDDTAVYFCAREGVDIMAVPWYFDY |
| | | WGQGTLVAVSS |
| MPK176 VH.1; MPK176 VH.2; | 234 | GYTLTAYG |
| MPK176 VH.3; MPK176 VH.4; | | |
| MPK176 VH.5; MPK176 VH.6; | | |
| MPK176 CDR-H1 (aa) | | |
| MPK176 VH.1; MPK176 VH.2; | 235 | ISPKHGNT |
| MPK176 VH.3; MPK176 VH.5; | | |
| MPK176 CDR-H2 (aa) | | |
| MPK176 VH.1; MPK176; | 236 | AREGVDIMAVPWYFDY |
| MPK176 VH.4 CDR-H3 (aa) | | |
| | 237-238 | See Sequence Listing |
| MPK176 VL.1; MPK176 VL.2; | 239 | NIGSKS |
| MPK176 VL.3; MPK176 VL.4; | | |
| MPK176 VL.5; MPK176 CDR- | | |
| L1 (aa) | | |
| MPK176 VL.1; MPK176 VL.3; | 240 | DDS |
| MPK176 VL.4; MPM2 VL.2; | | |
| MPM2 VL.6; MPK176; MPM2 | | |
| CDR-L2 (aa) | | |
| MPK176 VL.1; MPK176 VL.2; | 241 | QVWDSATDHWV |
| MPK176 VL.3; MPK176 VL.4; | | |
| MPK176 VL.5; MPK176 CDR- | | |
| L3 (aa) | | |
| | 242-267 | See Sequence Listing |
| MPK180; MPM2 VL.5; MPM2 | 268 | DAS |
| VL.9; MPK187; MPK197; MPP1 | | |
| CDR-L2 (aa) | | |
| | 269-289 | See Sequence Listing |
| MPK185 CDR-L2 (aa) | 290 | EVT |
| | 291-313 | See Sequence Listing |
| MPK188 CDR-L2 (aa) | 314 | GTS |
| | 315-338 | See Sequence Listing |
| MPK194-v2 CDR-L2 (aa) | 339 | DDD |
| | 340-355 | See Sequence Listing |
| MPK201 VH.1; MPK201 VH (nt) | 356 | GAGGTGCAGCTGGTGCAGTCTGGAGCAGAGGTGAAA |
| | | AAGCCCGGGGAGTCTCTGAAGATCTCCTGTCAGGGTT |
| | | CTGGATACAGCTTTATTAATTACTGGATCGGCTGGGTG |
| | | CGCCAGAAGCCCGGGAAAGGCCTGGAGTGGATGGGA |
| | | ATCATCTATCCTTTTGACTCTGAGACCAAATACAGCCC |
| | | GTCCTTCCAAGGCCACGTCACCTTCTCAGCCGACAAG |
| | | TCCATCAACACCGCCTACGTGCAGTGGAGCAGCCTGA |
| | | GGGCCTCGGACACCGCCATGTATTACTGTGCGAGATTT |
| | | ACCTTTGGTCTTGCAGTGACCACTGCTGATGTTAGTGG |
| | | GGAAGGCCAGGCTTTTGAAATCTGGGGCCAAGGGAC |
| | | AATGGTCACCGTCTCTTCAG |
| MPK201 VH.1; MPK201 VH | 357 | EVQLVQSGAEVKKPGESLKISCQGSGYSFINYWIGWVR |
| (aa) | | QKPGKGLEWMGIIYPFDSETKYSPSFQGHVTFSADKSIN |
| | | TAYVQWSSLRASDTAMYYCARFTFGLAVTTADVSGEG |
| | | QAFEIWGQGTMVTVSS |
| MPK201 VH.1; MPK201 VH.2; | 358 | ARFTFGLAVTTADVSGEGQAFEI |
| MPK201 VH.3; MPK201 VH.4; | | |
| MPK201 VH.5; MPK201 VH.6; | | |
| MPK201 CDR-H3 (aa) | | |
| MPK201 VK.1; MPK201 VL (nt) | 359 | GAAATAGTGATGATGCAGTCTCCAGCCACCCTGTCTGT |
| | | GTCTCCAGGGGAAAGAGCCACCCTCTCCTGCAGGGCC |
| | | AGTCAGAGTGTTGCCGGCAACTTAGCCTGGTACCAGC |
| | | AGAGACCTGGCCAGGCTCCCAGACTCCTCGTTTATGG |
| | | TGCATCCACCAGGGCCAGTGGTGTCCCAGCCAGGTTC |
| | | AGTGGCAGTGGGTCTGAGACAGAGTTCACTCTCACCA |
| | | TCAGCAGCCTGCAGTCTGAAGATTTTGCAGTTTATTAC |
| | | TGTCACCATTATAGTGATTGGCCTCAAGCCTTCGGCCA |
| | | AGGGACACGACTGGACATTAAAC |
| MPK201 VK.1; MPK201 VL (aa) | 360 | EIVMMQSPATLSVSPGERATLSCRASQSVAGNLAWYQQ |
| | | RPGQAPRLLVYGASTRASGVPARFSGSGSETEFTLTISSL |
| | | QSEDFAVYYCHHYSDWPQAFGQGTRLDIK |
| | 361-391 | See Sequence Listing |
| MPK127 CDR-L2 (aa) | 392 | ANN |
| | 393-408 | See Sequence Listing |
| MPK130 CDR-L2 (aa) | 409 | VNT |
| | 410-466 | See Sequence Listing |
| MPK150-v2; MPK158 CDR-L2 | 467 | RNS |
| (aa) | | |
| | 468-488 | See Sequence Listing |
| MPK155 CDR-L2 (aa) | 489 | RYT |
| | 490-509 | See Sequence Listing |
| MPK162 CDR-L2 (aa) | 510 | GNS |
| | 511-518 | See Sequence Listing |
| MPK 174-v2 CDR-L2 (aa) and | 519 | DNS |
| MPK 196 CDR-L2 (aa) | | |
| | 520-554 | See Sequence Listing |
| MPH12; MPH12 VL-FRGL- | 555 | GNI |
| D25E; MPH12 VL-FRGL-S26A; | | |
| MPH12 VL-FRGL-D25S; | | |
| MPH12 VL-S26A; MPH12 VL- | | |
| D25E; MPH12 VL-D25S; | | |
| MPH12 VL-FRGL CDR-L2 (aa) | | |
| | 556-582 | See Sequence Listing |
| RSD5-CD-L2 (aa) | 583 | DVS |
| | 584-623 | See Sequence Listing |
| HMB2416-CD-L2 (aa) | 624 | SDS |
| | 625-632 | See Sequence Listing |
| HMB3210-CD-L2 (aa) | 633 | DNN |
| | 634-653 | See Sequence Listing |
| HMB2430-CD-L2 (aa) | 654 | AND |
| | 655-678 | See Sequence Listing |
| Human IgG1 CH1-CH3-LS | 679 | ASTKGPSVFPLAPSSKSTSGGTAALGCLVKDYFPEPVTVS |
| mutation | | WNSGALTSGVHTFPAVLQSSGLYSLSSVVTVPSSSLGTQT |
| | | YICNVNHKPSNTKVDKKVEPKSCDKTHTCPPCPAPELLG |
| | | GPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSRDELTKNQVSLTCLVKGFYPSDIAVEWESNGQPENN |
| | | YKTTPPVLDSDGSFFLYSKLTVDKSRWQQGNVFSCSVLH |
| | | EALHSHYTQKSLSLSPGK |
| Human IgG1 hinge-CH2-CH3- | 680 | EPKSCDKTHTCPPCPAPELLGGPSVFLFPPKPKDTLMISR |
| LS mutation | | TPEVTCVVVDVSHEDPEVKFNWYVDGVEVHNAKTKPR |
| | | EEQYNSTYRVVSVLTVLHQDWLNGKEYKCKVSNKALP |
| | | APIEKTISKAKGQPREPQVYTLPPSRDELTKNQVSLTCLV |
| | | KGFYPSDIAVEWESNGQPENNYKTTPPVLDSDGSFFLYS |
| | | KLTVDKSRWQQGNVFSCSVLHEALHSHYTQKSLSLSPG |
| | | K |
| Human IgG1 CH3-LS mutation | 681 | GQPREPQVYTLPPSRDELTKNQVSLTCLVKGFYPSDIAVE |
| | | WESNGQPENNYKTTPPVLDSDGSFFLYSKLTVDKSRWQ |
| | | QGNVFSCSVLHEALHSHYTQKSLSLSPGK |
| IgG1m3 CH1-CH3-LS mutation | 682 | ASTKGPSVFPLAPSSKSTSGGTAALGCLVKDYFPEPVTVS |
| | | WNSGALTSGVHTFPAVLQSSGLYSLSSVVTVPSSSLGTQT |
| | | YICNVNHKPSNTKVDKRVEPKSCDKTHTCPPCPAPELLG |
| | | GPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSREEMTKNQVSLTCLVKGFYPSDIAVEWESNGQPENN |
| | | YKTTPPVLDSDGSFFLYSKLTVDKSRWQQGNVFSCSVLH |
| | | EALHSHYTQKSLSLSPGK |
| IgG1m17,1 CH1-CH3-LS | 683 | ASTKGPSVFPLAPSSKSTSGGTAALGCLVKDYFPEPVTVS |
| mutation | | WNSGALTSGVHTFPAVLQSSGLYSLSSVVTVPSSSLGTQT |
| | | YICNVNHKPSNTKVDKKVEPKSCDKTHTCPPCPAPELLG |
| | | GPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSRDELTKNQVSLTCLVKGFYPSDIAVEWESNGQPENN |
| | | YKTTPPVLDSDGSFFLYSKLTVDKSRWQQGNVFSCSVLH |
| | | EALHSHYTQKSLSLSPGK |
| IgG1m3,1 CH1-CH3-LS | 684 | ASTKGPSVFPLAPSSKSTSGGTAALGCLVKDYFPEPVTVS |
| mutation | | WNSGALTSGVHTFPAVLQSSGLYSLSSVVTVPSSSLGTQT |
| | | YICNVNHKPSNTKVDKRVEPKSCDKTHTCPPCPAPELLG |
| | | GPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSRDELTKNQVSLTCLVKGFYPSDIAVEWESNGQPENN |
| | | YKTTPPVLDSDGSFFLYSKLTVDKSRWQQGNVFSCSVLH |
| | | EALHSHYTQKSLSLSPGK |
| | 685-687 | See Sequence Listing |
| IgG1m3 CH1-CH3-LS mutation | 688 | ASTKGPSVFPLAPSSKSTSGGTAALGCLVKDYFPEPVTVS |
| | | WNSGALTSGVHTFPAVLQSSGLYSLSSVVTVPSSSLGTQT |
| | | YICNVNHKPSNTKVDKRVEPKSCDKTHTCPPCPAPELLG |
| | | GPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSREEMTKNQVSLTCLVKGFYPSDIAVEWESNGQPENN |
| | | YKTTPPVLDSDGSFFLYSKLTVDKSRWQQGNVFSCSVLH |
| | | EALHAHYTQKSLSLSPGK |
| IgG1m17,1 CH1-CH3-LS | 689 | ASTKGPSVFPLAPSSKSTSGGTAALGCLVKDYFPEPVTVS |
| mutation | | WNSGALTSGVHTFPAVLQSSGLYSLSSVVTVPSSSLGTQT |
| | | YICNVNHKPSNTKVDKKVEPKSCDKTHTCPPCPAPELLG |
| | | GPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSRDELTKNQVSLTCLVKGFYPSDIAVEWESNGQPENN |
| | | YKTTPPVLDSDGSFFLYSKLTVDKSRWQQGNVFSCSVLH |
| | | EALHAHYTQKSLSLSPGK |
| IgG1m3,1 CH1-CH3-LS | 690 | ASTKGPSVFPLAPSSKSTSGGTAALGCLVKDYFPEPVTVS |
| mutation | | WNSGALTSGVHTFPAVLQSSGLYSLSSVVTVPSSSLGTQT |
| | | YICNVNHKPSNTKVDKRVEPKSCDKTHTCPPCPAPELLG |
| | | GPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSRDELTKNQVSLTCLVKGFYPSDIAVEWESNGQPENN |
| | | YKTTPPVLDSDGSFFLYSKLTVDKSRWQQGNVFSCSVLH |
| | | EALHAHYTQKSLSLSPGK |
| | 691-699 | See Sequence Listing |
| MPK190-v1.1 VH (nt) and | 701 | CAGGTGCAGCTGGTGGAGTCTGGAGGAGGACTGGTG |
| MPK190-v1.3 VH (nt) | | AAGCCAGGAGGATCTCTGACCCTGTCCTGCGCCGCTA |
| | | GCGGCTTCAGCATCTCTGGCTACGGCATGAACTGGGT |
| | | GAGGCAGGCTCCAGGAAGGGGACTGGAGTGGGTGGC |
| | | CTCCATCTCCGCCTCCAGCACCTACATCTACTATGCTGA |
| | | CTCCATGAAGGGCAGATTCACCATCTCTCGCGATAATG |
| | | GCAAGACATCCCTGTATCTGCAGATGAAGAGCCTGAG |
| | | GGCCGAGGACACAGCCGTGTACTATTGTGCCAGAGCT |
| | | CCCATCGAGCCCCCTTATTTTGATCTGTGGGGCAGGGG |
| | | CACCCTGGTGACAGTGTCTTCC |
| MPK190-v1.1 VH (aa) and | 702 | QVQLVESGGGLVKPGGSLTLSCAASGFSISGYGMNWVR |
| MPK190-v1.3 VH (aa) | | QAPGRGLEWVASISASSTYIYYADSMKGRFTISRDNGKT |
| | | SLYLQMKSLRAEDTAVYYCARAPIEPPYFDLWGRGTLV |
| | | TVSS |
| MPK190-v1.3 VL (nt) | 703 | CAGTCCGTGCTGACCCAGCCACCTTCCGTGAGCGGAG |
| | | CTCCAGGCCAGAGGGTGACCATCAGCTGCACAGGCTC |
| | | CAGCTCTAACATCGGCGCCGGCTACGACGTGCACTGG |
| | | TATCAGCATCTGCCAGGCAAGGCTCCCAAGGTGCTGAT |
| | | CTACGGCAACACAAATAGACCTTCTGTGGTGCCAGAT |
| | | CGCTTCTCTGGCTCCAATAGCGGCGCCTCTGCTTCCCT |
| | | GGCTATCTCCGGACTGCAGGCTGAGGACGAGGCTGAT |
| | | TACTTCTGTCAGAGCTATGACAGGCGGCTGTCTGGCCC |
| | | TATCTTTGGCGGCGGCACCAAGCTGACAGTGCTG |
| MPK190-v1.3 VL (aa) | 704 | QSVLTQPPSVSGAPGQRVTISCTGSSSNIGAGYDVHWYQ |
| | | HLPGKAPKVLIYGNTNRPSVVPDRFSGSNSGASASLAIS |
| | | GLQAEDEADYFCQSYDRRLSGPIFGGGTKLTVL |
| MPK190-v1.3; MPK104-v1.3 | 705 | GNT |
| CDR-L2 (aa) | | |
| | 706-717 | See Sequence Listing |
| MPK190-G1m3-LS VH (nt) | 718 | GAGGTGCAGCTGGTGGAGTCGGGGGGAGGCCTGGTC |
| | | AAGCCGGGGGGGTCCCTGACACTCTCCTGTGCAGCCT |
| | | CTGGATTCTCCATCAGTGGATATGGCATGAACTGGG |
| | | TCCGCCAGGCTCCAGGGAGGGGACTGGAGTGGGTCG |
| | | CATCCATTAGTGCCAGTAGTACTTACATATATTATGCA |
| | | GACTCAATGAAGGGCCGATTCACCATCTCCAGAGACA |
| | | ACGGCAAGACTTCACTCTATCTGCAAATGAAGAGTTT |
| | | GCGAGCCGAGGACACGGCTGTCTATTATTGTGCGAGA |
| | | GCGCCTATAGAACCGCCGTACTTCGATCTCTGGGGC |
| | | CGTGGCACCCTGGTCACTGTCTCCTCAGCGTCGACCA |
| | | AGGGCCCATCGGTCTTCCCCCTGGCACCAAGTAGCAA |
| | | GAGCACATCCGGTGGCACAGCCGCCCTGGGTTGTCTG |
| | | GTGAAAGATTATTTCCCTGAGCCCGTGACAGTCTCCTG |
| | | GAACTCTGGCGCCCTGACCTCCGGAGTGCACACATTC |
| | | CCTGCTGTGCTGCAGTCCAGCGGCCTGTACTCCCTGTC |
| | | TTCCGTGGTGACCGTGCCAAGCTCTTCCCTGGGCACC |
| | | CAGACATATATCTGCAACGTGAATCACAAGCCTTCCAA |
| | | TACAAAGGTGGACAAGAGGGTGGAGCCAAAGAGCTG |
| | | TGATAAGACCCATACATGCCCACCTTGTCCAGCTCCAG |
| | | AGCTGCTGGGCGGCCCATCCGTGTTCCTGTTTCCACCC |
| | | AAGCCCAAGGACACCCTGATGATCTCTAGAACCCCAG |
| | | AGGTGACATGCGTGGTGGTGGACGTGTCCCACGAGGA |
| | | TCCCGAGGTGAAGTTTAACTGGTACGTGGATGGCGTG |
| | | GAGGTGCATAATGCTAAGACAAAGCCCAGGGAGGAGC |
| | | AGTACAACAGCACCTATCGGGTGGTGTCTGTGCTGAC |
| | | AGTGCTGCATCAGGACTGGCTGAACGGCAAGGAGTAT |
| | | AAGTGCAAGGTGAGCAATAAGGCCCTGCCTGCTCCAA |
| | | TCGAGAAGACCATCTCTAAGGCCAAGGGCCAGCCCAG |
| | | AGAGCCTCAGGTGTACACACTGCCTCCAAGCCGCGAG |
| | | GAGATGACCAAGAACCAGGTGTCTCTGACATGTCTGG |
| | | TGAAGGGCTTCTATCCCTCTGACATCGCTGTGGAGTGG |
| | | GAGTCCAATGGCCAGCCTGAGAACAATTACAAGACCA |
| | | CACCCCCTGTGCTGGACTCCGATGGCAGCTTCTTTCTG |
| | | TATTCCAAGCTGACCGTGGATAAGAGCAGGTGGCAGC |
| | | AGGGCAACGTGTTCTCCTGTTCTGTGCTGCACGAAGC |
| | | CCTGCACTCCCATTATACTCAGAAGTCCCTGTCCCTGT |
| | | CCCCTGGAAAA |
| MPK190-G1m3-LS VH (aa) | 719 | EVQLVESGGGLVKPGGSLTLSCAASGFSISGYGMNWVR |
| | | QAPGRGLEWVASISASSTYIYYADSMKGRFTISRDNGKT |
| | | SLYLQMKSLRAEDTAVYYCARAPIEPPYFDLWGRGTLV |
| | | TVSSASTKGPSVFPLAPSSKSTSGGTAALGCLVKDYFPEP |
| | | VTVSWNSGALTSGVHTFPAVLQSSGLYSLSSVVTVPSSSL |
| | | GTQTYICNVNHKPSNTKVDKRVEPKSCDKTHTCPPCPAP |
| | | ELLGGPSVFLFPPKPKDTLMISRTPEVTCVVVDVSHEDPE |
| | | VKFNWYVDGVEVHNAKTKPREEQYNSTYRVVSVLTVL |
| | | HQDWLNGKEYKCKVSNKALPAPIEKTISKAKGQPREPQ |
| | | VYTLPPSREEMTKNQVSLTCLVKGFYPSDIAVEWESNGQ |
| | | PENNYKTTPPVLDSDGSFFLYSKLTVDKSRWQQGNVFSC |
| | | SVLHEALHSHYTQKSLSLSPGK |
| MPK190-G1m3-LS VL (nt) | 720 | CAGTCTGTGCTGACGCAGCCGCCCTCAGTGTCTGGGG |
| | | CCCCCGGGCAGAGGGTCACCATCTCCTGCACAGGGAG |
| | | TAACTCCAACATCGGGGCAGGATATGATGTTCACTG |
| | | GTACCAACATCTTCCAGGAAAAGCCCCCAAAGTCCTC |
| | | ATCTATGGTAACAACAATCGGCCCTCAGTGGTCCCTG |
| | | ACCGATTCTCTGGCTCCAACTCTGGCGCCTCAGCCTCC |
| | | CTGGCCATCAGTGGGCTCCAGGCTGAGGATGAGGCTG |
| | | ATTATTTCTGCCAGTCTTATGACAGGAGACTGAGTG |
| | | GTCCGATCTTCGGCGGAGGGACCAAGCTGACCGTCC |
| | | TAGGGCAGCCCAAGGCTGCCCCCTCGGTCACTCTGTT |
| | | CCCGCCCTCGAGTGAGGAGCTCCAAGCCAACAAGGCC |
| | | ACCCTGGTCTGTCTGATTAGCGATTTCTACCCTGGTGC |
| | | TGTCACTGTCGCATGGAAGGCCGATTCATCCCCCGTCA |
| | | AAGCTGGTGTCGAGACAACAACCCCCAGCAAGCAGT |
| | | CTAACAATAAGTACGCCGCTTCCAGCTATCTGTCCCTG |
| | | ACACCTGAGCAGTGGAAGAGCCACAGGTCTTACTCCT |
| | | GTCAGGTCACTCACGAAGGATCAACTGTGGAAAAAAC |
| | | TGTGGCACCAACCGAATGTTCA |
| MPK190-G1m3-LS VL (aa) | 721 | QSVLTQPPSVSGAPGQRVTISCTGSNSNIGAGYDVHWY |
| | | QHLPGKAPKVLIYGNNNRPSVVPDRFSGSNSGASASLAI |
| | | SGLQAEDEADYFCQSYDRRLSGPIFGGGTKLTVLGQPK |
| | | AAPSVTLFPPSSEELQANKATLVCLISDFYPGAVTVAWKA |
| | | DSSPVKAGVETTTPSKQSNNKYAASSYLSLTPEQWKSHR |
| | | SYSCQVTHEGSTVEKTVAPTECS |
| MPK190-v1.3-Glm17,1-LS HC | 722 | CAGGTGCAGCTGGTGGAGTCTGGAGGAGGACTGGTG |
| (nt) | | AAGCCAGGAGGATCTCTGACCCTGTCCTGCGCCGCTA |
| | | GCGGCTTCAGCATCTCTGGCTACGGCATGAACTGGGT |
| | | GAGGCAGGCTCCAGGAAGGGGACTGGAGTGGGTGGC |
| | | CTCCATCTCCGCCTCCAGCACCTACATCTACTATGCTGA |
| | | CTCCATGAAGGGCAGATTCACCATCTCTCGCGATAATG |
| | | GCAAGACATCCCTGTATCTGCAGATGAAGAGCCTGAG |
| | | GGCCGAGGACACAGCCGTGTACTATTGTGCCAGAGCT |
| | | CCCATCGAGCCCCCTTATTTTGATCTGTGGGGCAGGGG |
| | | CACCCTGGTGACAGTGTCTTCCGCGTCGACCAAGGGC |
| | | CCATCGGTCTTCCCCCTGGCACCAAGTAGCAAGAGCA |
| | | CATCCGGTGGCACAGCCGCCCTGGGTTGTCTGGTGAA |
| | | AGATTATTTCCCTGAGCCCGTGACAGTCTCCTGGAACT |
| | | CTGGCGCCCTGACCTCCGGAGTGCACACATTCCCTGC |
| | | TGTGCTGCAGTCCAGCGGCCTGTACTCCCTGTCTTCCG |
| | | TGGTGACCGTGCCAAGCTCTTCCCTGGGCACCCAGAC |
| | | ATATATCTGCAACGTGAATCACAAGCCTTCCAATACAA |
| | | AGGTGGACAAGAAGGTGGAGCCAAAGAGCTGTGATA |
| | | AGACCCATACATGCCCACCTTGTCCAGCTCCAGAGCTG |
| | | CTGGGCGGCCCATCCGTGTTCCTGTTTCCACCCAAGCC |
| | | CAAGGACACCCTGATGATCTCTAGAACCCCAGAGGTG |
| | | ACATGCGTGGTGGTGGACGTGTCCCACGAGGATCCCG |
| | | AGGTGAAGTTTAACTGGTACGTGGATGGCGTGGAGGT |
| | | GCATAATGCTAAGACAAAGCCCAGGGAGGAGCAGTAC |
| | | AACAGCACCTATCGGGTGGTGTCTGTGCTGACAGTGC |
| | | TGCATCAGGACTGGCTGAACGGCAAGGAGTATAAGTG |
| | | CAAGGTGAGCAATAAGGCCCTGCCTGCTCCAATCGAG |
| | | AAGACCATCTCTAAGGCCAAGGGCCAGCCCAGAGAGC |
| | | CTCAGGTGTACACACTGCCTCCAAGCCGCGACGAGTT |
| | | GACCAAGAACCAGGTGTCTCTGACATGTCTGGTGAAG |
| | | GGCTTCTATCCCTCTGACATCGCTGTGGAGTGGGAGTC |
| | | CAATGGCCAGCCTGAGAACAATTACAAGACCACACCC |
| | | CCTGTGCTGGACTCCGATGGCAGCTTCTTTCTGTATTC |
| | | CAAGCTGACCGTGGATAAGAGCAGGTGGCAGCAGGG |
| | | CAACGTGTTCTCCTGTTCTGTGCTGCACGAAGCCCTGC |
| | | ACTCCCATTATACTCAGAAGTCCCTGTCCCTGTCCCCT |
| | | GGAAAA |
| MPK190-v1.3-G1m17,1-LS HC | 723 | QVQLVESGGGLVKPGGSLTLSCAASGFSISGYGMNWVR |
| (aa) | | QAPGRGLEWVASISASSTYIYYADSMKGRFTISRDNGKT |
| | | SLYLQMKSLRAEDTAVYYCARAPIEPPYFDLWGRGTLV |
| | | TVSSASTKGPSVFPLAPSSKSTSGGTAALGCLVKDYFPEP |
| | | VTVSWNSGALTSGVHTFPAVLQSSGLYSLSSVVTVPSSSL |
| | | GTQTYICNVNHKPSNTKVDKKVEPKSCDKTHTCPPCPAP |
| | | ELLGGPSVFLFPPKPKDTLMISRTPEVTCVVVDVSHEDPE |
| | | VKFNWYVDGVEVHNAKTKPREEQYNSTYRVVSVLTVL |
| | | HQDWLNGKEYKCKVSNKALPAPIEKTISKAKGQPREPQ |
| | | VYTLPPSRDELTKNQVSLTCLVKGFYPSDIAVEWESNGQ |
| | | PENNYKTTPPVLDSDGSFFLYSKLTVDKSRWQQGNVFSC |
| | | SVLHEALHSHYTQKSLSLSPGK |
| MPK190-v1.3-G1m17,1-LS LC | 724 | CAGTCCGTGCTGACCCAGCCACCTTCCGTGAGCGGAG |
| (nt)- | | CTCCAGGCCAGAGGGTGACCATCAGCTGCACAGGCTC |
| version 1 | | CAGCTCTAACATCGGCGCCGGCTACGACGTGCACTGG |
| | | TATCAGCATCTGCCAGGCAAGGCTCCCAAGGTGCTGAT |
| | | CTACGGCAACACAAATAGACCTTCTGTGGTGCCAGAT |
| | | CGCTTCTCTGGCTCCAATAGCGGCGCCTCTGCTTCCCT |
| | | GGCTATCTCCGGACTGCAGGCTGAGGACGAGGCTGAT |
| | | TACTTCTGTCAGAGCTATGACAGGCGGCTGTCTGGCCC |
| | | TATCTTTGGCGGCGGCACCAAGCTGACAGTGCTGGGG |
| | | CAGCCCAAGGCTGCCCCCTCGGTCACTCTGTTCCCGC |
| | | CCTCGAGTGAGGAGCTCCAAGCCAACAAGGCCACCCT |
| | | GGTCTGTCTGATTAGCGATTTCTACCCTGGTGCTGTCA |
| | | CTGTCGCATGGAAGGCCGATTCATCCCCCGTCAAAGCT |
| | | GGTGTCGAGACAACAACCCCCAGCAAGCAGTCTAACA |
| | | ATAAGTACGCCGCTTCCAGCTATCTGTCCCTGACACCT |
| | | GAGCAGTGGAAGAGCCACAGGTCTTACTCCTGTCAGG |
| | | TCACTCACGAAGGATCAACTGTGGAAAAAACTGTGGC |
| | | ACCAACCGAATGTTCA |
| MPK190-v1.3-G1m17,1-LS LC | 725 | QSVLTQPPSVSGAPGQRVTISCTGSSSNIGAGYDVHWYQ |
| (aa) | | HLPGKAPKVLIYGNTNRPSVVPDRFSGSNSGASASLAIS |
| | | GLQAEDEADYFCQSYDRRLSGPIFGGGTKLTVLGQPKA |
| | | APSVTLFPPSSEELQANKATLVCLISDFYPGAVTVAWKAD |
| | | SSPVKAGVETTTPSKQSNNKYAASSYLSLTPEQWKSHRS |
| | | YSCQVTHEGSTVEKTVAPTECS |
| | 726-734 | See Sequence Listing |
| MPM10 CDR-L2 (aa) | 734 | AAS |
| | 735-751 | See Sequence Listing |
| MPM8 CDR-L2 (aa) | 752 | KVS |
| | 753- | See Sequence Listing |
| MPO1 CDR-L2 (aa) | 762 | DVT |
| | 763-771 | See Sequence Listing |
| MPO7 CDR-L2 | 772 | EVF |
| | 773-790 | See Sequence Listing |
| MPP2 CDR-L2 (aa) | 791 | SAS |
| | 792-800 | See Sequence Listing |
| MPR16; MPR19-v2 CDR-L2 (aa) | 801 | DVI |
| | 802-810 | See Sequence Listing |
| MPK65-v2 VH (nt) MPK65 | 811 | CAAGTGCCACTAGTGCAAAGTGGTGCAGAAGTGAAG |
| VH.1 VH (VH.1) (hamster codon | | AAGCCTGGAGAGTCCCTGAAGATCTCCTGCAAGGGCT |
| optimized) | | CTGGCTACTCCTTCATCAACTACTGGATCGGCTGGGTG |
| | | CGGCAGATGCCTGGCAAGGGCCTGGAATGGATGGGCG |
| | | TGATCTACCCCTTCGACTCCGATACCAAGTATTCTCCTT |
| | | CTTTTCAAGGCCACGTGACCATCTCTGCCGACAAGTC |
| | | CTCCAACACCGCCTACGTGCAGTGGTCCAGCCTGAAA |
| | | GCTTCTGATACAGCTATGTACTACTGTGCTAGATTCACC |
| | | TTCGGCATCGCCGTGGTGACCGACGACGTGTCCGGCG |
| | | AGGGACAGGCCTTCGAGATCTGGGGCCAGGGCACCAT |
| | | GGTCACCGTGTCTAGC |
| | 812-813 | See Sequence Listing |
| MPK65 VH.2 N31S-D55Q CDR- | 814 | GYSFISYW |
| H1; MPK65 VH.3 CDR-H1; | | |
| MPK65 VH.4 CDR-H1; MPK65 | | |
| VH.5 CDR-H1; MPK65 VH.6; | | |
| MPK201 VH.4; MPK201 VH.5; | | |
| MPK201 VH.6 CDR-H1 (aa) | | |
| | 815 | See Sequence Listing |
| MPK65 VH.3 VH N31S-S56A | 816 | CAAGTGCCACTAGTGCAAAGTGGTGCAGAAGTGAAG |
| (nt) | | AAGCCTGGAGAGTCTCTGAAGATCTCCTGCAAGGGCT |
| | | CTGGCTACTCCTTCATCTCCTACTGGATCGGCTGGGTG |
| | | CGGCAGATGCCTGGCAAAGGCCTGGAATGGATGGGCG |
| | | TGATCTACCCCTTCGATGCTGATACCAAGTATTCTCCTT |
| | | CTTTTCAAGGCCACGTGACCATCTCTGCCGACAAGTC |
| | | CTCCAACACCGCCTACGTGCAGTGGTCCAGCCTGAAG |
| | | GCCTCCGACACCGCTATGTACTACTGTGCTAGATTCAC |
| | | CTTCGGCATCGCCGTGGTGACAGACGACGTGTCCGGC |
| | | GAGGGACAGGCCTTCGAGATCTGGGGCCAGGGCACC |
| | | ATGGTCACCGTGTCTAGC |
| MPK65 VH.3 VH N31S-S56A | 817 | QVPLVQSGAEVKKPGESLKISCKGSGYSFISYWIGWVRQ |
| (aa) | | MPGKGLEWMGVIYPFDADTKYSPSFQGHVTISADKSSN |
| | | TAYVQWSSLKASDTAMYYCARFTFGIAVVTDDVSGEG |
| | | QAFEIWGQGTMVTVSS |
| MPK65 VH.3 N31S-S56A CDR- | 818 | IYPFDADT |
| H2 (aa) | | |
| | 819-828 | See Sequence Listing |
| MPK176 VH.1 (nt) (hamster | 829 | CAAGTGCAACTAGTGCAAAGTGGTGCAGAAGTGAAG |
| codon optimized) | | AAACCTGGCGCTTCCGTGCGGGTGTCCTGCAAGGCCT |
| | | CTGGCTACACCCTGACCGCCTACGGCATCTCCTGGGTC |
| | | AGACAGGCTCCAGGACAGGGCCTGGAGTGGATGGGC |
| | | TGGATCTCTCCCAAGCACGGCAACACCCACTACGCCC |
| | | AGCGGTTCCAGGGCAGAGTGACCATGACCACCGATAC |
| | | CTCCACCTCTACAGCTTACATGGAACTGAGATCCCTGC |
| | | GGCCTGACGACACCGCTGTGTACTTTTGTGCCAGAGA |
| | | GGGCGTGGACATCATGGCCGTGCCTTGGTATTTCGACT |
| | | ACTGGGGCCAAGGAACACTGGTGGCCGTGTCTAGC |
| | 830-835 | See Sequence Listing |
| MPK176 VH.4 VH; MPK176 | 836 | CAAGTGCAACTAGTGCAAAGTGGTGCAGAAGTGAAG |
| VH.4 N57S (nt) | | AAACCTGGAGCTTCCGTGCGGGTGTCCTGCAAGGCCT |
| | | CTGGCTACACCCTGACCGCTTATGGCATCTCCTGGGTC |
| | | AGACAGGCTCCAGGACAAGGCCTGGAGTGGATGGGC |
| | | TGGATCTCTCCCAAGCACGGCTCTACCCACTACGCCCA |
| | | GCGGTTCCAGGGCAGAGTGACCATGACCACCGATACA |
| | | AGCACCTCCACAGCCTACATGGAACTGAGATCCCTGC |
| | | GGCCTGACGACACCGCCGTGTACTTTTGTGCCAGAGA |
| | | GGGCGTGGACATCATGGCCGTGCCTTGGTACTTCGACT |
| | | ACTGGGGCCAGGGCACCCTGGTGGCTGTGTCTAGC |
| MPK176 VH.4; MPK176 VH.4 | 837 | QVQLVQSGAEVKKPGASVRVSCKASGYTLTAYGISWVR |
| N57S (aa) | | QAPGQGLEWMGWISPKHGSTHYAQRFQGRVTMTTDTS |
| | | TSTAYMELRSLRPDDTAVYFCAREGVDIMAVPWYFDY |
| | | WGQGTLVAVSS |
| MPK176 VH.6; MPK176 VH.4; | 838 | ISPKHGST |
| MPK176 VH.4 N57S CDR-H2 | | |
| (aa) | | |
| | 839-844 | See Sequence Listing |
| MPK201 VK.1 (nt) (hamster | 845 | GAAATCGTGATGATGCAAAGTCCAGCAACTCTGTCTG |
| codon optimized) | | TGTCCCCTGGCGAGCGGGCTACACTGTCCTGCAGAGC |
| | | CTCTCAGTCCGTGGCCGGCAACCTGGCCTGGTACCAG |
| | | CAGCGGCCAGGACAAGCTCCTAGACTCCTGGTCTATG |
| | | GCGCTTCCACCAGAGCTTCTGGCGTGCCCGCCAGATT |
| | | CTCCGGAAGCGGCTCTGAAACCGAGTTCACCCTGACC |
| | | ATCTCCAGCCTGCAGTCTGAGGACTTCGCCGTGTACTA |
| | | CTGTCACCACTACTCCGATTGGCCTCAGGCCTTTGGCC |
| | | AGGGCACCCGGCTGGACATCAAG |
| MPK201 VK.2 E1Q-W94L; | 846 | CAAATCGTGATGATGCAAAGTCCAGCAACTCTCTCTGT |
| MPK201 VK.2 VL (nt) | | GTCCCCCGGCGAGCGGGCTACACTGTCCTGCAGAGCC |
| | | TCTCAGTCCGTGGCCGGCAACCTGGCCTGGTACCAGC |
| | | AGCGGCCTGGCCAAGCTCCTAGACTGCTGGTCTATGG |
| | | CGCTTCCACCAGAGCTTCTGGCGTGCCAGCCAGATTC |
| | | TCCGGCTCTGGAAGCGAAACCGAGTTCACCCTGACCA |
| | | TCTCCAGCCTGCAGTCTGAGGACTTCGCCGTGTACTAC |
| | | TGTCACCACTACTCCGATCTGCCTCAGGCCTTTGGCCA |
| | | GGGCACCCGGCTGGACATCAAG |
| MPK201 VK.2 E1Q-W94L; | 847 | QIVMMQSPATLSVSPGERATLSCRASQSVAGNLAWYQQ |
| MPK201 VK.2 VL (aa) | | RPGQAPRLLVYGASTRASGVPARFSGSGSETEFTLTISSL |
| | | QSEDFAVYYCHHYSDLPQAFGQGTRLDIK |
| MPK201 VK.2 E1Q-W94L; | 848 | HHYSDLPQA |
| MPK201 VK.2 CDRL3 (aa) | | |
| MPK65 VK.1 VL (nt) (hamster | 849 | GAAATCGTGATGATGCAAAGTCCAGCAACTCTGTCCG |
| codon optimized) | | TGTCCCCCGGCGAGAGAGCTACACTGTCTTGTAGAGC |
| | | CTCTCAATCTGTGGCCGGCAACCTGGCCTGGTATCAGC |
| | | AGCGGCCTGGCCAGGCTCCTCGGCTGCTGATCTACGG |
| | | CGCTTCTACCCGGGCTACCGGCGTCCCAGCCAGATTCT |
| | | CCGGCTCTGGCAGCGAGACAGAATTCACCCTGACCAT |
| | | CTCCAGCCTGCAGTCCGAGGACTTCGCCGTGTACTACT |
| | | GCCACCAGTACTCCAACTGGCCTCAGGCCTTTGGACA |
| | | GGGCACCAGACTCGATATCAAG |
| MPK65 VK.2 VL; E1Q-W94L | 850 | CAAATCGTGATGATGCAAAGTCCAGCAACTCTGTCCG |
| (nt) | | TGTCCCCCGGCGAGCGGGCCACCCTCTCTTGTAGAGC |
| | | CTCTCAGAGCGTCGCTGGCAACCTGGCCTGGTATCAG |
| | | CAGCGGCCTGGCCAAGCTCCTAGACTGCTGATCTACG |
| | | GCGCTTCTACCAGAGCTACCGGCGTGCCAGCCAGATT |
| | | CTCCGGCTCTGGATCTGAAACCGAGTTCACCCTGACC |
| | | ATCTCCAGCCTGCAGTCCGAGGACTTCGCCGTGTACTA |
| | | CTGCCACCAGTACTCCAACCTGCCTCAGGCCTTTGGCC |
| | | AGGGCACACGGCTGGATATCAAG |
| MPK65 VK.2 VL; E1Q-W94L | 851 | QIVMMQSPATLSVSPGERATLSCRASQSVAGNLAWYQQ |
| (aa) | | RPGQAPRLLIYGASTRATGVPARFSGSGSETEFTLTISSLQ |
| | | SEDFAVYYCHQYSNLPQAFGQGTRLDIK |
| MPK65 VK.2 E1Q-W94L CDR- | 852 | HQYSNLPQA |
| L3 (aa) | | |
| | 853-855 | See Sequence Listing |
| MPK176 VL.2 E3V-D49Q; | 856 | QDS |
| MPK176 VL.2; MPM2 VL.3; | | |
| MPK176 VL.5; MPM2 VL.7 | | |
| CDR-L2 (aa) | | |
| MPK176 VL.3 E3V-D52K; | 857 | AGTTATGTGCTAACTCAACCACCAAGTGTGTCTGTGGC |
| MPK176 VL.3 VL (nt) | | CCCCGGACAGACCGCCAGAATCAGCTGTGGCGGCAAC |
| | | AACATCGGCTCTAAGTCCGTGCACTGGTACCAGCAGA |
| | | AACCTGGCCAGGCCCCTGTGGTGGTCGTGTACGACGA |
| | | CTCCAAGCGGCCATCTGGCATCCCTGAGAGATTTAGCG |
| | | GCTCCAACTCCGGCAATACCGCTACACTGACCATCTCC |
| | | GGCGTGGAAGCTGGCGACGAGGCCGACTACTTCTGCC |
| | | AAGTGTGGGATTCTGCTACCGATCACTGGGTGTTCGGC |
| | | GGAGGCACCAAGCTGACCGTGCTG |
| MPK176 VL.3 E3V-D52K; | 858 | SYVLTQPPSVSVAPGQTARISCGGNNIGSKSVHWYQQKP |
| MPK176 VL.3 VL (aa) | | GQAPVVVVYDDSKRPSGIPERFSGSNSGNTATLTISGVEA |
| | | GDEADYFCQVWDSATDHWVFGGGTKLTVL |
| | 859-870 | See Sequence Listing |
| MPM2 VL.4 D49K; MPM2 | 871 | KDS |
| VL.4; MPM2 VL.8 CRD-L2 (aa) | | |
| | 872-890 | See Sequence Listing |
| MPK201 VH.1 (nt) (hamster | 891 | GAAGTGCAACTAGTGCAAAGTGGTGCAGAAGTGAAG |
| codon optimized) | | AAGCCCGGCGAGTCCCTGAAGATCAGCTGTCAGGGAT |
| | | CTGGCTACTCCTTCATCAACTACTGGATCGGCTGGGTG |
| | | CGGCAGAAACCTGGCAAGGGCCTGGAATGGATGGGC |
| | | ATCATCTACCCTTTCGACTCCGAGACAAAGTACTCTCC |
| | | TAGCTTCCAGGGCCACGTGACCTTCTCCGCCGACAAG |
| | | TCCATCAACACCGCTTATGTGCAGTGGTCCTCTCTGAG |
| | | AGCCTCTGACACCGCCATGTACTACTGCGCCAGATTCA |
| | | CCTTTGGACTGGCTGTCACAACCGCTGATGTGTCTGGC |
| | | GAAGGCCAGGCCTTCGAGATCTGGGGCCAAGGCACC |
| | | ATGGTGACCGTGTCCAGC |
| | 892-894 | See Sequence Listing |
| MPK201 VH.3 E1Q D55S; | 895 | CAAGTGCAACTAGTGCAAAGTGGTGCAGAAGTGAAG |
| MPK201 VH.3 VH (nt) | | AAGCCTGGCGAGTCCCTGAAGATCTCCTGCCAGGGAT |
| | | CTGGATATAGCTTCATCAACTACTGGATCGGCTGGGTG |
| | | CGGCAGAAACCCGGCAAGGGCCTGGAATGGATGGGC |
| | | ATCATCTACCCTTTCTCCTCCGAGACCAAGTACTCTCC |
| | | TTCTTTCCAGGGCCACGTGACATTTTCTGCTGATAAGT |
| | | CCATCAACACAGCCTACGTGCAGTGGTCCTCTCTGAG |
| | | AGCCTCCGACACCGCTATGTACTACTGTGCTAGATTCA |
| | | CCTTCGGCCTGGCCGTGACCACCGCCGACGTGTCTGG |
| | | CGAAGGCCAGGCCTTCGAGATCTGGGGCCAAGGCACC |
| | | ATGGTCACCGTGTCCAGC |
| MPK201 VH.3 E1Q D55S; | 896 | QVQLVQSGAEVKKPGESLKISCQGSGYSFINYWIGWVR |
| MPK201 VH.3 VH (aa) | | QKPGKGLEWMGIIYPFSSETKYSPSFQGHVTFSADKSIN |
| | | TAYVQWSSLRASDTAMYYCARFTFGLAVTTADVSGEG |
| | | QAFEIWGQGTMVTVSS |
| MPK201 VH.3 E1Q D55S; | 897 | IYPFSSET |
| MPK201 VH.3; MPK201 VH.6 | | |
| CDR-H2 (aa) | | |
| MPK201 VH.4 E1Q N31S; | 898 | CAAGTGCAACTAGTGCAAAGTGGTGCAGAAGTGAAG |
| MPK201 VH.4- VH (nt) | | AAGCCCGGCGAGTCTCTGAAGATCTCTTGTCAGGGCT |
| | | CTGGCTACTCCTTCATCTCCTACTGGATCGGCTGGGTG |
| | | CGGCAGAAGCCTGGCAAAGGACTGGAATGGATGGGC |
| | | ATCATCTATCCTTTCGACTCCGAGACAAAGTACTCTCC |
| | | TTCTTTCCAGGGCCACGTGACCTTCTCCGCCGACAAG |
| | | TCCATCAACACCGCCTACGTGCAGTGGTCCAGCCTGA |
| | | GAGCTTCCGACACCGCTATGTACTACTGCGCCAGATTC |
| | | ACCTTTGGCCTGGCCGTGACAACCGCTGATGTGTCCG |
| | | GCGAAGGCCAGGCCTTCGAGATCTGGGGACAAGGCA |
| | | CCATGGTCACCGTGTCTAGC |
| MPK201 VH.4 E1Q N31S; | 899 | QVQLVQSGAEVKKPGESLKISCQGSGYSFISYWIGWVR |
| MPK201 VH.4 VH (aa) | | QKPGKGLEWMGITYPFDSETKYSPSFQGHVTFSADKSIN |
| | | TAYVQWSSLRASDTAMYYCARFTFGLAVTTADVSGEG |
| | | QAFEIWGQGTMVTVSS |
| | 900-903 | See Sequence Listing |
| MPK190-v1.3-rlgG1m17,1-LS | 904 | CAGTCCGTGCTGACCCAGCCACCTTCCGTGAGCGGAG |
| LC (nt) - version 2 | | CTCCAGGCCAGAGGGTGACCATCAGCTGCACAGGCTC |
| | | CAGCTCTAACATCGGCGCCGGCTACGACGTGCACTGG |
| | | TATCAGCATCTGCCAGGCAAGGCTCCCAAGGTGCTGAT |
| | | CTACGGCAACACAAATAGACCTTCTGTGGTGCCAGAT |
| | | CGCTTCTCTGGCTCCAATAGCGGCGCCTCTGCTTCCCT |
| | | GGCTATCTCCGGACTGCAGGCTGAGGACGAGGCTGAT |
| | | TACTTCTGTCAGAGCTATGACAGGCGGCTGTCTGGCCC |
| | | TATCTTTGGCGGCGGCACCAAGCTGACAGTGCTGGGT |
| | | CAGCCCAAGGCTGCCCCCTCGGTCACTCTGTTCCCGC |
| | | CCTCGAGTGAGGAGCTCCAAGCCAACAAGGCCACCCT |
| | | GGTCTGTCTGATTAGCGATTTCTACCCTGGTGCTGTCA |
| | | CTGTCGCATGGAAGGCCGATTCATCCCCCGTCAAAGCT |
| | | GGTGTCGAGACAACAACCCCCAGCAAGCAGTCTAACA |
| | | ATAAGTACGCCGCTTCCAGCTATCTGTCCCTGACACCT |
| | | GAGCAGTGGAAGAGCCACAGGTCTTACTCCTGTCAGG |
| | | TCACTCACGAAGGATCAACTGTGGAAAAAACTGTGGC |
| | | ACCAACCGAATGTTCA |
| Wild Type Kappa Light Chain CL | 905 | RTVAAPSVFIFPPSDEQLKSGTASVVCLLNNFYPREAKVQ |
| | | WKVDNALQSGNSQESVTEQDSKDSTYSLSSTLTLSKAD |
| | | YEKHKVYACEVTHQGLSSPVTKSFNRGEC |

*Table 1 indicates CDRH1, CDRH2 and CDRH3 (IMGT definition), in that order, in bold in each corresponding VH sequence; and CDRLI, CDRL2, CDRL3, in that order, in bold in each corresponding VL sequence. A VH, VL or CDR identified in Table 1 for one antibody may also be present in another antibody not specifically identified in Table 1, but referenced in Table 2.

Sequences referenced in Table 2, Table 3, Table 4, or elsewhere in this specification, but not included in Table 1, may be found in the Sequence Listing.

TABLE 2
Sequence Key - SEQ ID NOs.: of certain antibodies and virus specificity

| Ab, VH, or | VH | VH | CDR | CDR | CDR | VL | VL | CDR | CDR | CDR | |
|---|---|---|---|---|---|---|---|---|---|---|---|
| VL | nt | aa | H1 aa | H2 aa | H3 aa | nt | aa | L1 aa | L2 aa | L3 aa | Virus |
| MPK9 | 128 | 129 | 130 | 47 | 131 | 132 | 133 | 18 | 117 | 134 | RSV/MPV |
| MPK10 | 11 | 12 | 13 | 14 | 15 | 16 | 17 | 18 | 19 | 20 | RSV/MPV |
| MPK15 | 100 | 101 | 102 | 103 | 104 | 105 | 106 | 107 | 108 | 109 | MPV |
| MPK18 | 37 | 38 | 39 | 40 | 25 | 41 | 42 | 18 | 43 | 44 | RSV/MPV |
| MPK30-v1 | 45 | 46 | 39 | 47 | 48 | 49 | 50 | 18 | 19 | 51 | RSV/MPV |
| MPK36-v3 | 52 | 53 | 54 | 24 | 25 | 55 | 56 | 18 | 43 | 57 | RSV/MPV |
| MPK44 | 1 | 2 | 3 | 4 | 5 | 6 | 7 | 8 | 9 | 10 | RSV |
| MPK51 | 58 | 59 | 60 | 61 | 62 | 63 | 64 | 65 | 66 | 67 | RSV/MPV |
| MPK51- | 715 | 716 | 60 | 61 | 62 | 717 | 64 | 65 | 66 | 67 | RSV/MPV |
| v1.1 | | | | | | | | | | | |
| MPK65-v2 | 135 | 136 | 137 | 138 | 139 | 140 | 141 | 142 | 143 | 144 | RSV |
| MPK65-v2 | 811 | | | | | | | | | | RSV |
| VH.1 | | | | | | | | | | | |
| (hamster | | | | | | | | | | | |
| codon | | | | | | | | | | | |
| optimised) | | | | | | | | | | | |
| MPK65-v2 | 812 | 813 | 814 | 815 | 139 | | | | | | RSV |
| VH.2 | | | | | | | | | | | |
| MPK65-v2 | 816 | 817 | 814 | 818 | 139 | | | | | | RSV |
| VH.3 | | | | | | | | | | | |
| MPK65-v2 | 819 | 820 | 814 | 138 | 821 | | | | | | RSV |
| VH.4 | | | | | | | | | | | |
| MPK65-v2 | 822 | 823 | 814 | 138 | 824 | | | | | | RSV |
| VH.5 | | | | | | | | | | | |
| MPK65-v2 | 825 | 826 | 814 | 815 | 821 | | | | | | RSV |
| VH.6 | | | | | | | | | | | |
| MPK65-v2 | 827 | 828 | 814 | 815 | 824 | | | | | | RSV |
| VH.7 | | | | | | | | | | | |
| MPK65-v2 | | | | | | 849 | | | | | RSV |
| VK.1 | | | | | | | | | | | |
| (hamster | | | | | | | | | | | |
| codon | | | | | | | | | | | |
| optimized) | | | | | | | | | | | |
| MPK65-v2 | | | | | | 850 | 851 | 142 | 143 | 852 | RSV |
| VK.2 | | | | | | | | | | | |
| MPK65v2- | 135 | 136 | 137 | 138 | 139 | 850 | 851 | 142 | 143 | 852 | RSV |
| v1.2 | | | | | | | | | | | |
| MPK65v2- | 816 | 817 | 814 | 818 | 139 | 140 | 141 | 142 | 143 | 144 | RSV |
| v3.1 | | | | | | | | | | | |
| MPK67 | 68 | 69 | 39 | 40 | 25 | 70 | 71 | 18 | 43 | 44 | RSV/MPV |
| MPK73 | 119 | 120 | 121 | 122 | 123 | 124 | 125 | 126 | 117 | 127 | RSV/MPV |
| MPK77 | 72 | 73 | 74 | 24 | 48 | 75 | 76 | 18 | 19 | 77 | RSV/MPV |
| MPK77- | 711 | 712 | 74 | 24 | 48 | 713 | 76 | 18 | 19 | 77 | RSV/MPV |
| v1.1 | | | | | | | | | | | |
| MPK86 | 78 | 79 | 80 | 81 | 25 | 82 | 83 | 18 | 19 | 84 | RSV/MPV |
| MPK92 | 85 | 86 | 87 | 88 | 89 | 90 | 91 | 92 | 19 | 93 | RSV/MPV |
| MPK99 | 94 | 95 | 39 | 96 | 48 | 97 | 98 | 99 | 19 | 44 | RSV/MPV |
| MPK102 | 21 | 22 | 23 | 24 | 25 | 26 | 27 | 18 | 19 | 28 | RSV/MPV |
| MPK104 | 110 | 111 | 112 | 113 | 114 | 115 | 116 | 18 | 117 | 118 | RSV/MPV |
| MPK104- | 706 | 707 | 112 | 113 | 114 | 708 | 116 | 18 | 117 | 118 | RSV/MPV |
| v1.1 | | | | | | | | | | | |
| MPK104- | 706 | 707 | 112 | 113 | 114 | 709 | 710 | 18 | 705 | 118 | RSV/MPV |
| v1.3 | | | | | | | | | | | |
| MPK108 | 29 | 30 | 31 | 32 | 33 | 34 | 35 | 18 | 19 | 36 | RSV/MPV |
| MPK126 | 377 | 378 | 380 | 81 | 25 | 381 | 382 | 383 | 19 | 384 | RSV/MPV |
| MPK127 | 385 | 386 | 387 | 388 | 389 | 390 | 391 | 18 | 392 | 393 | RSV/MPV |
| MPK129 | 394 | 395 | 396 | 397 | 398 | 399 | 400 | 401 | 19 | 402 | RSV/MPV |
| MPK130 | 403 | 404 | 80 | 405 | 406 | 407 | 408 | 18 | 409 | 410 | RSV/MPV |
| MPK132- | 411 | 412 | 87 | 88 | 89 | 413 | 414 | 401 | 19 | 402 | RSV/MPV |
| v1 | | | | | | | | | | | |
| MPK133 | 415 | 416 | 417 | 24 | 48 | 418 | 419 | 18 | 19 | 420 | RSV/MPV |
| MPK136 | 421 | 422 | 396 | 397 | 398 | 423 | 424 | 401 | 19 | 402 | RSV/MPV |
| MPK141 | 425 | 426 | 39 | 40 | 427 | 428 | 429 | 401 | 19 | 28 | RSV/MPV |
| MPK142- | 430 | 431 | 130 | 47 | 432 | 433 | 434 | 18 | 117 | 134 | RSV/MPV |
| v1 | | | | | | | | | | | |
| MPK142- | 430 | 431 | 130 | 47 | 432 | 435 | 436 | 437 | 143 | 438 | RSV/MPV |
| v2 | | | | | | | | | | | |
| MPK144 | 439 | 440 | 441 | 442 | 48 | 443 | 444 | 445 | 19 | 446 | RSV/MPV |
| MPK145 | 447 | 431 | 130 | 47 | 432 | 448 | 449 | 18 | 117 | 134 | RSV/MPV |
| MPK146 | 450 | 451 | 452 | 453 | 48 | 454 | 455 | 456 | 19 | 384 | RSV/MPV |
| MPK149 | 457 | 458 | 459 | 24 | 25 | 460 | 461 | 18 | 19 | 402 | RSV/MPV |
| MPK150- | 462 | 463 | 87 | 464 | 89 | 465 | 466 | 401 | 467 | 468 | RSV/MPV |
| v2 | | | | | | | | | | | |
| MPK151 | 469 | 470 | 471 | 81 | 25 | 472 | 473 | 445 | 19 | 384 | RSV/MPV |
| MPK152 | 474 | 475 | 471 | 40 | 476 | 477 | 478 | 18 | 19 | 51 | RSV/MPV |
| MPK153 | 479 | 480 | 481 | 88 | 48 | 482 | 483 | 18 | 19 | 51 | RSV/MPV |
| MPK155 | 484 | 485 | 87 | 88 | 25 | 486 | 487 | 488 | 489 | 402 | RSV/MPV |
| MPK157 | 490 | 491 | 492 | 47 | 493 | 494 | 495 | 18 | 19 | 384 | RSV/MPV |
| MPK158 | 496 | 497 | 498 | 47 | 499 | 500 | 501 | 18 | 467 | 502 | RSV/MPV |
| MPK161- | 145 | 146 | 147 | 138 | 148 | 149 | 150 | 151 | 152 | 153 | RSV |
| v1 | | | | | | | | | | | |
| MPK161- | 145 | 146 | 147 | 138 | 148 | 154 | 155 | 142 | 156 | 157 | RSV |
| v2 | | | | | | | | | | | |
| MPK162 | 503 | 504 | 505 | 506 | 507 | 508 | 509 | 18 | 510 | 511 | RSV/MPV |
| MPK163 | 158 | 159 | 160 | 161 | 162 | 163 | 164 | 165 | 166 | 167 | RSV |
| MPK165 | 168 | 169 | 170 | 138 | 148 | 171 | 172 | 173 | 156 | 157 | RSV |
| MPK167 | 174 | 175 | 137 | 138 | 176 | 177 | 178 | 142 | 143 | 179 | RSV |
| MPK168 | 180 | 181 | 182 | 138 | 183 | 184 | 185 | 186 | 143 | 187 | RSV |
| MPK169- | 188 | 189 | 137 | 138 | 190 | 191 | 192 | 193 | 143 | 194 | RSV |
| v2 | | | | | | | | | | | |
| MPK170 | 195 | 196 | 137 | 138 | 197 | 198 | 199 | 142 | 200 | 157 | RSV |
| MPK171- | 201 | 202 | 137 | 138 | 203 | 204 | 205 | 206 | 207 | 208 | RSV |
| v1 | | | | | | | | | | | |
| MPK171- | 209 | 210 | 137 | 138 | 203 | 211 | 212 | 142 | 143 | 213 | RSV |
| v2 | | | | | | | | | | | |
| MPK173 | 214 | 215 | 216 | 217 | 218 | 219 | 220 | 221 | 222 | 223 | RSV |
| MPK174- | 512 | 513 | 514 | 515 | 516 | 517 | 518 | 18 | 519 | 520 | RSV/MPV |
| v2 | | | | | | | | | | | |
| MPK175 | 224 | 225 | 137 | 226 | 227 | 228 | 229 | 230 | 143 | 231 | RSV |
| MPK176 | 232 | 233 | 234 | 235 | 236 | 237 | 238 | 239 | 240 | 241 | RSV |
| MPK176 | 829 | | | | | | | | | | RSV |
| VH.1 | | | | | | | | | | | |
| (hamster | | | | | | | | | | | |
| codon | | | | | | | | | | | |
| optimized) | | | | | | | | | | | |
| MPK176 | 830 | 831 | 234 | 235 | 832 | | | | | | RSV |
| VH.2 | | | | | | | | | | | |
| MPK176 | 833 | 834 | 234 | 235 | 835 | | | | | | RSV |
| VH.3 | | | | | | | | | | | |
| MPK176 | 836 | 837 | 234 | 838 | 236 | | | | | | RSV |
| VH.4 | | | | | | | | | | | |
| MPK176 | 839 | 840 | 234 | 235 | 841 | | | | | | RSV |
| VH.5 | | | | | | | | | | | |
| MPK176 | 842 | 843 | 234 | 838 | 844 | | | | | | RSV |
| VH.6 | | | | | | | | | | | |
| MPK176 | | | | | | 853 | | | | | RSV |
| VL.1 | | | | | | | | | | | |
| (hamster | | | | | | | | | | | |
| codon | | | | | | | | | | | |
| optimized) | | | | | | | | | | | |
| MPK176 | | | | | | 854 | 855 | 239 | 856 | 241 | RSV |
| VL.2 | | | | | | | | | | | |
| MPK176 | | | | | | 857 | 858 | 239 | 240 | 241 | RSV |
| VL.3 | | | | | | | | | | | |
| MPK176 | | | | | | 859 | 860 | 239 | 240 | 241 | RSV |
| VL.4 | | | | | | | | | | | |
| MPK176 | | | | | | 861 | 862 | 239 | 856 | 241 | RSV |
| VL.5 | | | | | | | | | | | |
| MPK176- | 232 | 233 | 234 | 235 | 236 | 857 | 858 | 239 | 240 | 241 | RSV |
| v1.3 | | | | | | | | | | | |
| MPK176- | 836 | 837 | 234 | 838 | 236 | 857 | 858 | 239 | 240 | 241 | RSV |
| v.4.3 | | | | | | | | | | | |
| MPK177 | 242 | 243 | 244 | 138 | 245 | 246 | 247 | 173 | 143 | 248 | RSV |
| MPK178 | 249 | 250 | 182 | 138 | 148 | 251 | 252 | 142 | 156 | 157 | RSV |
| MPK179- | 253 | 254 | 137 | 138 | 255 | 256 | 257 | 258 | 156 | 259 | RSV |
| v4 | | | | | | | | | | | |
| MPK180 | 260 | 261 | 262 | 263 | 264 | 265 | 266 | 267 | 268 | 269 | RSV |
| MPK181 | 270 | 271 | 137 | 138 | 272 | 273 | 274 | 173 | 143 | 275 | RSV |
| MPK182 | 276 | 277 | 278 | 279 | 280 | 281 | 282 | 206 | 207 | 208 | RSV |
| MPK185 | 283 | 284 | 285 | 138 | 286 | 287 | 288 | 289 | 290 | 291 | RSV |
| MPK186 | 292 | 293 | 137 | 226 | 294 | 295 | 296 | 297 | 143 | 298 | RSV |
| MPK187 | 299 | 300 | 301 | 302 | 303 | 304 | 305 | 306 | 268 | 307 | RSV |
| MPK188 | 308 | 309 | 137 | 138 | 310 | 311 | 312 | 313 | 314 | 157 | RSV |
| MPK189 | 315 | 316 | 137 | 138 | 317 | 318 | 319 | 142 | 143 | 213 | RSV |
| MPK190 | 119 | 120 | 121 | 122 | 123 | 521 | 522 | 126 | 117 | 127 | RSV/MPV |
| MPK190- | 701 | 702 | 121 | 122 | 123 | 714 | 522 | 126 | 117 | 127 | RSV/MPV |
| v1.1 | | | | | | | | | | | |
| MPK190- | 701 | 702 | 121 | 122 | 123 | 703 | 704 | 18 | 705 | 127 | RSV/MPV |
| v1.3 | | | | | | | | | | | |
| MPK191 | 320 | 321 | 137 | 138 | 322 | 323 | 324 | 142 | 143 | 325 | RSV |
| MPK193 | 326 | 327 | 170 | 138 | 328 | 329 | 330 | 142 | 143 | 157 | RSV |
| MPK194- | 331 | 332 | 333 | 334 | 335 | 336 | 337 | 338 | 339 | 340 | RSV |
| v2 | | | | | | | | | | | |
| MPK195 | 341 | 342 | 137 | 138 | 343 | 344 | 345 | 173 | 143 | 213 | RSV |
| MPK196 | 523 | 524 | 514 | 515 | 516 | 525 | 518 | 18 | 519 | 520 | RSV/MPV |
| MPK197 | 346 | 347 | 301 | 302 | 303 | 348 | 349 | 350 | 268 | 307 | RSV |
| MPK198 | 351 | 352 | 137 | 138 | 353 | 354 | 355 | 142 | 143 | 213 | RSV |
| MPK201 | 356 | 357 | 137 | 226 | 358 | 359 | 360 | 142 | 143 | 231 | RSV |
| MPK201 | 891 | | | | | | | | | | RSV |
| VH.1 | | | | | | | | | | | |
| (hamster | | | | | | | | | | | |
| codon | | | | | | | | | | | |
| optimized) | | | | | | | | | | | |
| MPK201 | 892 | 893 | 137 | 894 | 358 | | | | | | RSV |
| VH.2 | | | | | | | | | | | |
| MPK201 | 895 | 896 | 137 | 897 | 358 | | | | | | RSV |
| VH.3 | | | | | | | | | | | |
| MPK201 | 898 | 899 | 814 | 226 | 358 | | | | | | RSV |
| VH.4 | | | | | | | | | | | |
| MPK201 | 900 | 901 | 814 | 894 | 358 | | | | | | RSV |
| VH.5 | | | | | | | | | | | |
| MPK201 | 902 | 903 | 814 | 897 | 358 | | | | | | RSV |
| VH.6 | | | | | | | | | | | |
| MPK201 | | | | | | 845 | | | | | RSV |
| VK.1 | | | | | | | | | | | |
| (hamster | | | | | | | | | | | |
| codon | | | | | | | | | | | |
| optimized) | | | | | | | | | | | |
| MPK201 | | | | | | 846 | 847 | 142 | 143 | 848 | RSV |
| VK.2 | | | | | | | | | | | |
| MPK201- | 356 | 357 | 137 | 226 | 358 | 846 | 847 | 142 | 143 | 848 | RSV |
| v1.2 | | | | | | | | | | | |
| MPK201- | 898 | 899 | 814 | 226 | 358 | 359 | 360 | 142 | 143 | 231 | RSV |
| v4.1 | | | | | | | | | | | |
| MPK202 | 361 | 362 | 363 | 364 | 365 | 366 | 367 | 8 | 9 | 10 | RSV |
| MPK203 | 368 | 369 | 370 | 371 | 372 | 373 | 374 | 375 | 143 | 376 | RSV |
| MPK204 | 526 | 527 | 528 | 88 | 25 | 529 | 530 | 401 | 19 | 402 | RSV/MPV |
| MPM10 | 726 | 727 | 728 | 729 | 730 | 731 | 732 | 733 | 734 | 735 | RSV |
| MPM2 | 736 | 737 | 738 | 739 | 740 | 741 | 742 | 743 | 240 | 744 | RSV |
| MPM2 | 882 | 883 | 738 | 884 | 740 | | | | | | RSV |
| VH.2 | | | | | | | | | | | |
| MPM2 | 885 | 886 | 887 | 884 | 740 | | | | | | RSV |
| VH.4 | | | | | | | | | | | |
| MPM2 | 888 | 889 | 890 | 884 | 740 | | | | | | RSV |
| VH.5 | | | | | | | | | | | |
| MPM2 | | | | | | 863 | | | | | RSV |
| VL.1 | | | | | | | | | | | |
| (hamster | | | | | | | | | | | |
| codon | | | | | | | | | | | |
| optimized | | | | | | | | | | | |
| MPM2 | | | | | | 864 | 865 | 743 | 240 | 866 | RSV |
| VL.2 | | | | | | | | | | | |
| MPM2 | | | | | | 867 | 868 | 743 | 856 | 744 | RSV |
| VL.3 | | | | | | | | | | | |
| MPM2 | | | | | | 869 | 870 | 743 | 871 | 744 | RSV |
| VL.4 | | | | | | | | | | | |
| MPM2 | | | | | | 872 | 873 | 743 | 268 | 744 | RSV |
| VL.5 | | | | | | | | | | | |
| MPM2 | | | | | | 874 | 875 | 743 | 240 | 744 | RSV |
| VL.6 | | | | | | | | | | | |
| MPM2 | | | | | | 876 | 877 | 743 | 856 | 744 | RSV |
| VL.7 | | | | | | | | | | | |
| MPM2 | | | | | | 878 | 879 | 743 | 871 | 744 | RSV |
| VL.8 | | | | | | | | | | | |
| MPM2 | | | | | | 880 | 881 | 743 | 268 | 866 | RSV |
| VL.9 | | | | | | | | | | | |
| MPM8 | 745 | 746 | 747 | 748 | 749 | 750 | 751 | 221 | 752 | 753 | RSV |
| MPO1 | 754 | 755 | 756 | 757 | 758 | 759 | 760 | 761 | 762 | 763 | RSV |
| MPO7 | 764 | 765 | 766 | 767 | 768 | 769 | 770 | 771 | 772 | 773 | RSV |
| MPP1 | 774 | 775 | 776 | 777 | 778 | 779 | 780 | 781 | 268 | 782 | RSV |
| MPP2 | 783 | 784 | 785 | 786 | 787 | 788 | 789 | 790 | 791 | 792 | RSV |
| MPR16 | 793 | 794 | 795 | 796 | 797 | 798 | 799 | 800 | 801 | 802 | RSV |
| MPR19-v2 | 803 | 804 | 805 | 796 | 806 | 807 | 808 | 809 | 801 | 810 | RSV |
| MPH12 | 531 | 532 | 533 | 534 | 535 | | | | | | |
| VH | | | | | | | | | | | |
| MPH12 | 536 | 537 | 533 | 534 | 535 | | | | | | |
| VH-FRGL | | | | | | | | | | | |
| MPH12 | 538 | 539 | 533 | 540 | 535 | | | | | | |
| VH-D62E | | | | | | | | | | | |
| MPH12 | 541 | 542 | 533 | 543 | 535 | | | | | | |
| VH-S63A | | | | | | | | | | | |
| MPH12 | 544 | 545 | 533 | 540 | 535 | | | | | | |
| VH- | | | | | | | | | | | |
| FRGL- | | | | | | | | | | | |
| D62E | | | | | | | | | | | |
| MPH12 | 546 | 547 | 533 | 548 | 535 | | | | | | |
| VH- | | | | | | | | | | | |
| FRGL- | | | | | | | | | | | |
| S63A | | | | | | | | | | | |
| MPH12 | 549 | 550 | 533 | 534 | 551 | | | | | | |
| VH-2CP | | | | | | | | | | | |
| (C111P, | | | | | | | | | | | |
| C112.2P) | | | | | | | | | | | |
| MPH12 | | | | | | 552 | 553 | 554 | 555 | 556 | |
| VL | | | | | | | | | | | |
| MPH12 | | | | | | 557 | 558 | 554 | 555 | 556 | |
| VL-FRGL | | | | | | | | | | | |
| MPH12 | | | | | | 559 | 560 | 561 | 555 | 556 | |
| VL-D25S | | | | | | | | | | | |
| MPH12 | | | | | | 562 | 563 | 564 | 555 | 556 | |
| VL-D25E | | | | | | | | | | | |
| MPH12 | | | | | | 566 | 567 | 568 | 555 | 556 | |
| VL-S26A | | | | | | | | | | | |
| MPH12 | | | | | | 569 | 570 | 561 | 555 | 556 | |
| VL- | | | | | | | | | | | |
| FRGL- | | | | | | | | | | | |
| D25S | | | | | | | | | | | |
| MPH12 | | | | | | 571 | 572 | 564 | 555 | 556 | |
| VL- | | | | | | | | | | | |
| FRGL- | | | | | | | | | | | |
| D25E | | | | | | | | | | | |
| MPH12 | | | | | | 573 | 574 | 568 | 555 | 556 | |
| VL- | | | | | | | | | | | |
| FRGL- | | | | | | | | | | | |
| S26A | | | | | | | | | | | |

TABLE 3
Sequence Key - SEQ ID NOs.: of certain anti-RSV antibodies*

| | VH | VH | CDR | CDR | CDR | VL | VL | CDR | CDR | CDR |
|---|---|---|---|---|---|---|---|---|---|---|
| Ab, VH, or VL | nt | aa | H1 aa | H2 aa | H3 aa | nt | aa | L1 aa | L2 aa | L3 aa |
| RSD5 | 575 | 576 | 577 | 578 | 579 | 580 | 581 | 582 | 583 | 584 |
| RSD5-FRGL | 585 | 586 | 577 | 578 | 579 | 587 | 588 | 582 | 583 | 584 |
| RSD5-v2 | 575 | 576 | 577 | 578 | 579 | 587 | 588 | 582 | 583 | 584 |
| RSD5-v3 | 585 | 586 | 577 | 578 | 579 | 580 | 581 | 582 | 583 | 584 |
| HMB2437 | 590 | 591 | 592 | 593 | 594 | 595 | 596 | 597 | 143 | 598 |
| HMB2437-FRGL | 599 | 600 | 592 | 593 | 594 | 601 | 602 | 597 | 143 | 598 |
| HMB2437-v2 | 590 | 591 | 592 | 593 | 594 | 601 | 602 | 597 | 143 | 598 |
| HMB2437-v3 | 599 | 600 | 592 | 593 | 594 | 595 | 596 | 597 | 143 | 598 |
| HMB2432 | 603 | 604 | 605 | 606 | 607 | 608 | 609 | 610 | 268 | 611 |
| HMB2432-v2 | 612 | 613 | 605 | 606 | 607 | 614 | 615 | 610 | 268 | 611 |
| HMB2432-v3 | 603 | 604 | 605 | 606 | 607 | 614 | 615 | 610 | 268 | 611 |
| HMB2432-v4 | 612 | 613 | 605 | 606 | 607 | 608 | 609 | 610 | 268 | 611 |
| HMB2416 | 616 | 617 | 618 | 619 | 620 | 621 | 622 | 623 | 624 | 625 |

*Table 3 includes antibodies provided in U.S. Pat. No. 10,047,145, incorporated by reference herein in its entirety.

TABLE 4
Sequence Key - SEQ ID NOs.: of certain anti-RSV and/or anti-MPV antibodies*

| | VH | VH | CDR | CDR | CDR | VL | VL | CDR | CDR | CDR |
|---|---|---|---|---|---|---|---|---|---|---|
| Ab, VH, or VL | nt | aa | H1 aa | H2 aa | H3 aa | nt | aa | L1 aa | L2 aa | L3 aa |
| HMB3210 | 626 | 627 | 628 | 629 | 630 | 631 | 632 | 18 | 633 | 634 |
| HMB3210-v2 | 635 | 636 | 628 | 629 | 630 | 631 | 632 | 18 | 633 | 634 |
| HMB3210-v3 | 635 | 636 | 628 | 629 | 630 | 637 | 638 | 18 | 633 | 639 |
| HMB3210-v4 | 640 | 641 | 628 | 642 | 643 | 644 | 645 | 18 | 510 | 646 |
| HMB3210-v5 | 640 | 641 | 628 | 642 | 643 | 631 | 632 | 18 | 633 | 634 |
| HMB3210-v6 | 635 | 636 | 628 | 629 | 630 | 644 | 645 | 18 | 510 | 646 |
| HMB430 | 647 | 648 | 649 | 650 | 651 | 652 | 653 | 18 | 654 | 655 |
| HMB430-v2 | 656 | 657 | 649 | 650 | 651 | 652 | 653 | 18 | 654 | 655 |
| HMB430-v3 | 658 | 659 | 628 | 642 | 660 | 661 | 662 | 18 | 510 | 663 |
| HMB430-v4 | 658 | 659 | 629 | 642 | 660 | 652 | 653 | 18 | 654 | 655 |
| HMB430-v5 | 656 | 657 | 649 | 650 | 651 | 652 | 653 | 18 | 654 | 655 |

*Table 4 includes antibodies provided in U.S. Pat. No. 9,489,531, incorporated by reference herein in its entirety.

### EXAMPLES

### Example 1

### Neutralization of RSV

Antibodies were tested for neutralization against RSV. In some experiments, comparator antibodies MPE33, MPE8 (Corti et al. Nature. 2013 Sep. 19; 501(7467):439-43. doi: 10.1038/nature12442. Epub 2013 Aug. 18), MPF5, and RSD5 (see e.g. Jones et al. PLoS Patho. 15(7):e1007944 (2019); doi:10.1371/journal.ppat.1007944) were also tested.

In further detail, the following settings were used:

Conditions— Format: 384 well plate (microscopy plates from TTP Labtech); Replicates: 4 replicates; Infection medium (IM): MEM 2.4% Hyclone+P/S; Pre-incubation mAb-virus: 45 min at 37° C.; mAb concentration: 2500 ng/ml in IM (final is 625 ng/ml)→1:2 serial dilutions (titration horizontal, 11× points); Vvirus input: 350 TCID50; Cells: HEp-2, 1000 cells/well; Volumes: 10 ul mAb (in IM)+10 ul virus (in IM)+20 ul cells (in IM) (+10 ul detection solution); Spreading time: 6 days; Detection solution: Draq5 5× (1:300 in IM)→final 1:1500; Detection solution incubation time: 4h

Protocol— day 0: Make serial dilution of the mAb in IM and add 10 ul/well; Dilute virus in IM and add 10 ul/well; Incubate 45 min at 37° C.; Add cells 20 ul/well in IM; Incubate for time needed for the virus to spread—day 7: Add 10 ul Detection solution; Incubate and read at the appropriate time.

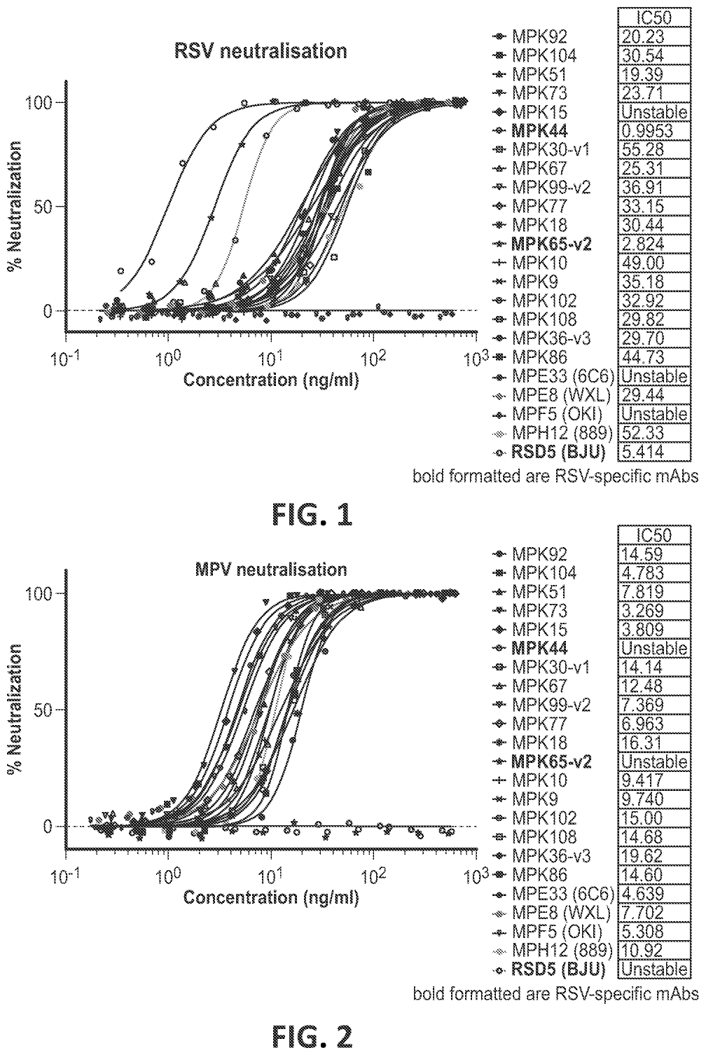

Results are shown in FIG. 1 and Table 5. IC50 values are in ng/mL.

For the 18 mAbs tested in Experiment A, 17 out of 18 mAbs showed potent neutralization of RSV (range IC50: 2.4-39.5 ng/ml). Neutralization was comparable to that of MPE8 (IC50: 29.44 ng/ml) and MPH12 (IC50: 52.33 ng/ml). Two mAbs (MPK44 and MPK65-v2) showed higher neutralizing activity than RSD5 (5.414 ng/ml).

For the 42 mAbs tested in Experiment C, all neutralized RSV with IC50 values ranging from 2.8 to 162.4 ng/ml. Of these, 34 mAbs (83%) showed potent neutralization (<30 ng/ml) and 15 (37%) showed very potent neutralization (<15 ng/ml).

MPK44, MPK65-v2, and RSD5 were RSV-specific.

MED18897 is further described in connection with the clinical study by AstraZeneca, “A Phase 2/3 Randomized, Double-blind, Palivizumab-controlled Study to Evaluate the Safety of MED18897, a Monoclonal Antibody With an Extended Half-life Against Respiratory Syncytial Virus, in High-risk Children (MEDLEY).”

TABLE 5
RSV Neutralization - IC50 (ng/ml)

| | | | | | |
|---|---|---|---|---|---|
| mAb | IC50 | mAb | IC50 | mAb | IC50 |

Experiment A

| | | | | | |
|---|---|---|---|---|---|
| MPK92 | 20.23 | MPK104 | 30.54 | MPK51 | 19.39 |
| MPK73 | 23.71 | MPK15 | Unstable | MPK44 | 0.9953 |
| MPK30-v-1 | 55.28 | MPK67 | 25.31 | MPK99-v2 | 36.91 |
| MPK77 | 33.15 | MPK18 | 30.44 | MPK65-v2 | 2.824 |
| MPK10 | 49.00 | MPK9 | 35.18 | MPK102 | 32.92 |
| MPK108 | 29.82 | MPK36-v3 | 29.70 | MPK86 | 44.73 |
| MPE33(6C6) | Unstable | MPE8 (WXL) | 29.44 | MPF5 (OKI) | Unstable |
| MPH12 (889) | 52.33 | RSD5 (BJU) | 5.414 | | |

Experiment B

| | | | | | |
|---|---|---|---|---|---|
| MPK161-v1 | 2.8 | MPK161-v2 | 1.1 | MPK170 | 1.1 |
| MPK163 | 1.4 | MPK171-v1 | 4.3 | MPK177 | 1.0 |
| MPK165 | 1.0 | MPK171-v2 | 1.0 | MPK178 | 1.0 |
| MPK167 | 1.0 | MPK173 | 10.2 | MPK179-v4 | 6.6 |
| MPK168 | 1.3 | MPK175 | 1.6 | MPK180 | 1.5 |
| MPK169-v2 | 1.4 | MPK176 | 4.2 | MPK186 | 1.2 |
| MPK188 | 1.0 | MPK195 | 1.1 | MPK187 | 9.1 |
| MPK189 | 1.2 | MPK197 | 14.7 | MPK203 | 2.0 |
| MPK191 | 1.2 | MPK198 | 1.2 | MPK194-v2 | 2.6 |
| MPK193 | 1.0 | MEDI8897 | 0.8 | palivizumab | 155.9 |
| MPK201 | 2.4 | MPK202 | 0.5 | | |

Experiment C

| | | | | | |
|---|---|---|---|---|---|
| MPK9 | 14.1 | MPK126 | 26.4 | MPK151 | 37.5 |
| MPK10 | 31.1 | MPK127 | 78.7 | MPK152 | 30.8 |
| MPK18 | 13.9 | MPK129 | 14.9 | MPK153 | 29.6 |
| MPK30-v1 | 16.0 | MPK130 | 20.6 | MPK155 | 26.5 |
| MPK36-v3 | 16.3 | MPK132-v1 | 18.0 | MPK157 | 24.1 |
| MPK51 | 10.0 | MPK133 | 21.1 | MPK158 | 18.2 |
| MPK67 | 14.3 | MPK136 | 34.4 | MPK162 | 25.2 |
| MPK73 | 11.4 | MPK141 | 14.0 | MPK174-v2 | 13.6 |
| MPK77 | 14.3 | MPK142-v1 | 21.1 | MPK190 | 9.5 |
| MPK86 | 27.6 | MPK142-v2 | 162.4 | MPK196 | 19.4 |
| MPK92 | 10.4 | MPK144 | 8.2 | MPK204 | 12.1 |
| MPK99 | 24.8 | MPK145 | 23.9 | MPH12 | 30.1 |
| MPK102 | 18.3 | MPK146 | 16.6 | MPE8 | 15.3 |
| MPK104 | 13.2 | MPK149 | 33.5 | MEDI8897 | 0.8 |
| MPK108 | 24.5 | MPK150-v2 | 14.7 | palivizumab | 155.9 |

### Example 2

### RSV Neutralization Breadth by MPK44 and MPK65-v2

The ability of two mAbs to neutralize RSV was explored in further detail using the following settings:

Materials— RSV viruses indicated in the protocol section; Hep2 cells (ATCC Cat. #ATCC/TSR CCL-23 Lot. 70023387) passage 5 for this experiment; Complete medium for Hep2 cells: EMEM (ATCC)+10% FBS (Seradigm Cat #97068-085 Lot #345K19 Heat Inactivated)+Pen/Strep (Gibco); Vero-TMPRSS2 cells (Electronic Laboratory Notebook ID: X007157); Growth medium: DMEM (Gibco Cat. #11995), 10% FBS (VWR Cat. #97068-085 Lot #345K19), 8. 1% Penicillin/Streptomycin (Gibco Cat. #15140-122), 8 g/mL of puromycin (Gibco Cat. #A1113803); Infection medium was the same as the growth medium; Paraformaldehyde, 16% w/v q. soln, methanol free (Alfa Aesar Cat. #43368); Mouse anti-RSV Blend for detection of F, G and NP proteins of both A and B strains (Millipore Cat. MAB858-4 Lot. 3439213); Goat anti-Mouse AF647 (Invitrogen, Cat. #A-21235); Hoechst nuclei dye (Thermo Fisher Cat. #62249); Corning® 96-well Flat Clear Bottom Black Polystyrene TC-treated Microplates (Cat. 3904)

Protocol— Day 0: Plate 20,000 Hep2 cells/well and Vero-TMPRSS2 cells in different 96-well plates; —Day 1: Prepare the antibodies proteins in infection medium and make serial dilutions in deep 96-well plates; Prepare virus dilution in a plastic bottle or falcon tube: 1) RSV A2 BPR-344-00 (BEI NR-12149), P2 (21-Oct-21); Titre (PFU/mL):1.03E+07; 20,000 cells/well; MOI:0.02; PFU/well:400; Virus volume/well (μL):0.04; Vol for 1500 wells (μL):58.54; Complete with medium (μL): 149941.46; 2) RSV B1 BPR-348-00 (BEI NR-4052), P2 (16-Nov-21); Titre (PFU/mL):3.25E+07; 20,000 cells/well; MOI:0.02; PFU/well:400; Virus volume/well (μL):0.01; Vol for 1500 wells (μL):18.46; Complete with medium (μL):149981.54; Using the multichannel pipette, transfer 20 μL of the Ab dilutions to the plated virus; Add 180 μL of the virus for each virus dilution on 96-well plates according to the plate above; Incubate for 30 mins at 37° C.; Aspirate the medium from the cells and transfer 90 μL of the virus/ab complex to the cells; Aspirate the virus and add 100 uL/well of 2.4% Microcrystalline cellulose resuspended in the respective culture medium for each cell line; Incubate for 48h in growth conditions; —Day 3: Aspirate the supernatant from the cells with vacuset; Fix the cells with 4% PFA for 30 mins in PFA at room temp; Wash two times with PBS; Add 50 μl/well of primary antibody diluted 1:1,000 in PBS containing 2% BSA and 0.05% Triton X-100; Incubate for 1h at room temp; Wash 3× with PBS; Add secondary anti-mouse AF647 antibody diluted 1:1,000 and nuclei dye Hoescht 1:1,000 in the same buffer as primary antibody; Incubate for 1h at room temp; Wash 3× with PBS and leave in 100 μl/well of PBS; Continue with quantification of infected cells in the Cytation5 plate reader.

Results are presented in Table 6. The mAbs neutralized different RSV strains with IC50 values ranging from 0.1 to 0.7 ng/ml.

TABLE 6
RSV Neutralization by MPK44 and MPK65-v2 - IC50 (ng/ml)

| | RSV A2 | RSV B1 BPR-348-00 |
|---|---|---|
| mAb | (BEI NR-12149) | (BEI NR-4052) |
| MPK44 | 0.1 | 0.7 |
| MPK65-v2 | 0.5 | 0.4 |
| RSD5 | 0.8 | 1.0 |
| MEDI8897 | 0.2 | 0.4 |

### Example 3

### Neutralization of MPV D280

Antibodies were tested for neutralization against MPV D280.

In further detail, the following settings were used:

Conditions— Format: 384 well plate (microscopy plates from TTP Labtech); Replicates: 4×; Infection medium (IM): MEM 2.4% Hyclone+P/S; Pre-incubation mAb-virus: 45 min at 37° C.; mAb concentration: 2500 ng/ml in IM (final is 500 ng/ml)->1:2 serial dilutions (titration horizontal, 1Ix points); virus input: 300 TCID50; Cells: HEp-2, 1000 cells/well; Volumes: 10 ul mAb (in IM)+10 ul virus (in IM)+10 ul cells (in IM)+20 ul Trypsin TPCK (in IM) (+10 ul detection solution); Spreading time: 6 days; Trypsin TPCK: 100 μg/ml in IM→40 μg/ml final; Detection solution: Draq5 6× (1:250 in IM)→final 1:1500; Detection solution incubation: 4h.

Protocol— day 0: Make serial dilution of the mAb in IM and add 10 ul/well; Dilute virus in IM and add 10 μl/well; Incubate 45 min at 37° C.; Add cells 10 ul/well in IM; Incubate for time needed for the virus to spread; —day 1: Add 20 ul/well Trypsin TPCK in IM; —day 7: Add 10 ul Detection solution; Incubate and read at the appropriate time.

Results are shown in FIG. 2 and Table 7 (which includes results from two separate assays for MPK15, MPE33, and MPF5).

In Experiment A, 16 out of 18 mAbs showed potent neutralization of MPV D280 (range IC50: 7-53 ng/ml). Neutralization was comparable to that of MPE8 (IC50: 7.7 ng/ml) and MPH12 (IC50: 10.92 ng/ml). Two mAbs (MPK44 and MPK65-v2) did not neutralize MPV D280, indicating that they are specific only for RSV. MPK15 showed potent neutralization (IC50=7 ng/ml) only against MPV D280, indicating that it is specific only for MPV D280.

In Experiment B, MPK15 potently neutralized MPV D280 (IC50 value: 5.8 ng/ml).

In Experiment C, 42 mAbs neutralized MPV D280 with IC50 values ranging from 1.3 to 135.1 ng/ml. Of these, 39 mAbs (95%) showed potent neutralization (<30 ng/ml) and 24 (59%) showed very potent neutralization (<15 ng/ml).

TABLE 7
MPV D280 Neutralization - IC50 (ng/ml)

| | | | | | |
|---|---|---|---|---|---|
| mAb | IC50 | mAb | IC50 | mAb | IC50 |

Experiment A

| | | | | | |
|---|---|---|---|---|---|
| MPK92 | 14.59 | MPK104 | 4.783 | MPK51 | 7.819 |
| MPK73 | 3.269 | MPK15 | 3.809 | MPK44 | Unstable |
| MPK30-v-1 | 14.14 | MPK67 | 12.48 | MPK99-v2 | 7.369 |
| MPK77 | 6.963 | MPK18 | 16.31 | MPK65-v2 | Unstable |
| MPK10 | 9.417 | MPK9 | 9.740 | MPK102 | 15.00 |
| MPK108 | 14.68 | MPK36-v3 | 19.62 | MPK86 | 14.60 |
| MPE33(6C6) | 4.639 | MPE8 (WXL) | 7.702 | MPF5 (OKI) | 5.308 |
| MPH12 (889) | 10.92 | RSD5 (BJU) | Unstable | | |

Experiment B

| | | | | | |
|---|---|---|---|---|---|
| MPK15 | 3.4 | MPR33 | 5.2 | MPF5 | 5.2 |

Experiment C

| | | | | | |
|---|---|---|---|---|---|
| MPK9 | 7.0 | MPK126 | 23.6 | MPK151 | 17.8 |
| MPK10 | 10.6 | MPK127 | 24.3 | MPK152 | 15.2 |
| MPK18 | 16.0 | MPK129 | 8.1 | MPK153 | 19.9 |
| MPK30-v1 | 15.7 | MPK130 | 4.3 | MPK155 | 14.1 |
| MPK36-v3 | 12.7 | MPK132-v1 | 10.1 | MPK157 | 7.9 |
| MPK51 | 8.8 | MPK133 | 3.5 | MPK158 | 6.0 |
| MPK67 | 15.3 | MPK136 | 15.8 | MPK162 | 101.7 |
| MPK73 | 2.7 | MPK141 | 9.8 | MPK174-v2 | 17.7 |
| MPK77 | 4.6 | MPK142-v1 | 3.0 | MPK190 | 1.3 |
| MPK86 | 14.0 | MPK142-v2 | 135.1 | MPK196 | 27.0 |
| MPK92 | 19.9 | MPK144 | 4.9 | MPK204 | 6.0 |
| MPK99 | 6.9 | MPK145 | 7.8 | MPH12 | 7.6 |
| MPK102 | 15.5 | MPK146 | 5.4 | MPE8 | 6.9 |
| MPK104 | 2.9 | MPK149 | 19.2 | MEDI8897 | No neutr. |
| MPK108 | 11.5 | MPK150-v2 | 15.8 | palivizumab | No neutr. |

### Example 4

### MPV D280 Neutralization Breadth by MPK15

The ability of MPK15 to neutralize MPV D280 was explored in further detail using the following settings:

Conditions— MPV viruses indicated in the protocol section; Hep2 cells (ATCC Cat. #ATCC/TSR CCL-23 Lot. 70023387) passage 5 for this experiment; Complete medium for Hep2 cells: EMEM (ATCC)+1000 FBS (Seradigm Cat #97068-085 Lot #345K19 Heat Inactivated)+Pen/Strep (Gibco); Vero-TMPRSS2 cells (Electronic Laboratory Notebook ID: X007157); Growth medium: DMEM (Gibco Cat. #11995), 10% FBS (VWR Cat. #97068-085 Lot #345K19), 8. 1% Penicillin/Streptomycin (Gibco Cat. #15140-122), 8 g/mL of puromycin (Gibco Cat. 4A1113803); Infection medium is the same as the growth medium; Paraformaldehyde, 16% w/v q. soln,. methanol free (Alfa Aesar Cat. #43368); Mouse anti-MPV antibody blend FITC-Conjugated (Quidel Cat. 4 01-035005); Goat anti-Mouse AF647 (Invitrogen, Cat. 4A-21235); Hoechst nuclei dye (Thermo Fisher Cat. #62249); Corning R 96-well Flat Clear Bottom Black Polystyrene TC-treated Microplates (Cat. 3904).

Protocol— day 0: Plate 20,000 Hep2 cells/well and Vero-TMPRSS2 cells in different 96-well plates; —day 1: Prepare the antibodies proteins in infection medium and make serial dilutions in deep 96-well plates; Prepare virus dilution in a plastic bottle or falcon tube: 1) Human Metapneumovirus-GFP CAN97-83 (Vira Tree M121; a MPV D280 strain); P2 (27 Sep. 2021); Titer (PFU/mL): 3.30E+05; # of cells/well: 20,000; MOI: 0.02; PFU/well: 400; Virus volume/well (μL): 1.21; Vol. for 1500 well (μL): 1818.18; Complete with medium (μL): 14181.82; 2) Human metapneumovirus-TN/96-213 (BEI NR-22243; a MPV D280 strain); P2 (27 Sep. 2021); Titer (PFU/mL); 3.35E+05; # of cells/well: 20,000; MOI: 0.02; PFU/well: 400; Virus volume/well (μL): 1.19; Vol. for 1500 wells (μL): 1791.04; Complete with medium (μL): 148208.96.; Using the multichannel pipette, transfer 20 μL of the Ab dilutions to the plated virus; Add 180 μL of the virus for each virus dilution on 96-well plates according to the plate above; Incubate for 30 mins at 37° C.; Aspirate the medium from the cells and transfer 90 μL of the virus/ab complex to the cells; Aspirate the virus and add 100 uL/well of 2.4% Microcrystalline cellulose resuspended in the respective culture medium for each cell line; Incubate for 48h in growth conditions; —day 3: Aspirate the supernatant from the cells with vacuset; Fix the cells with 4% PFA for 30 mins in PFA at room temp; Wash two times with PBS; Add 50 ul/well of primary antibody diluted 1:20 in PBS containing 2% BSA and 0.05% Triton X-100; Incubate for 1h at room temp; Wash 3× with PBS; Add secondary anti-mouse AF647 antibody diluted 1:1,000 and nuclei dye Hoescht 1:1,000 in the same buffer as primary antibody; Incubate for 1h at room temp; Wash 3× with PBS and leave in 100 ul/well of PBS; Continue with quantification of infected cells in the Cytation5 plate reader.

MPK15 potently neutralized different MPV D280 strains, as shown in Table 8.

TABLE 8
MPV D280 Neutralization by MPK15

| | MPV A2-GFP CAN97-83 | MPV B2-TN/96-213 |
|---|---|---|
| mAb | (Vira Tree M121) | (BEI NR-22243) |
| MPK15 | 5.8 | 15.3 |
| MPE33 | 82.2 | 30.0 |
| MPF5 | 11.5 | 12.2 |

### Example 5

### RSV and MPV D280 Neutralization Breadth

The ability of 16 mAbs to neutralize RSV and MPV D280 was explored in further detail using the following settings:

Conditions—RSV and MPV D280 viruses indicated in the protocol section; Hep2 cells (ATCC Cat. #ATCC/TSR CCL-23 Lot. 70023387) passage 5 for this experiment; Complete medium for Hep2 cells: EMEM (ATCC)+10% FBS (Seradigm Cat #97068-085 Lot #345K19 Heat Inactivated)+Pen/Strep (Gibco); Vero-TMPRSS2 cells (Electronic Laboratory Notebook ID: X007157); Growth medium: DMEM (Gibco Cat. #11995), 10% FBS (VWR Cat. #97068-085 Lot #345K19), 1% Penicillin/Streptomycin (Gibco Cat. #15140-122), 8 g/mL of puromycin (Gibco Cat. #A1113803); Infection medium is the same as the growth medium; Paraformaldehyde, 16% w/v q. soln,. methanol free (AlfaAesar Cat. #43368); Mouse anti-RSV Blend for detection of F, G and NP proteins of both A and B strains (Millipore Cat. MAB858-4 Lot. 3439213); Mouse anti-MPV antibody blend FITC-Conjugated (Quidel Cat. #01-035005); Goat anti-Mouse AF647 (Invitrogen, Cat. #A-21235); Hoechst nuclei dye (Thermo Fisher Cat. #62249); Corning® 96-well Flat Clear Bottom Black Polystyrene TC-treated Microplates (Cat. 3904).

Protocol— day 0: Plate 20,000 Hep2 cells/well and Vero-TMPRSS2 cells in different 96-well plates; —day 1: Prepare the antibodies proteins in infection medium and make serial dilutions in deep 96-well plates; Prepare virus dilution in a plastic bottle or falcon tube: 1) RSV A2 BPR-344-00 (BEI NR-12149), P2 (21-Oct-21); Titre (PFU/mL):1.03E+07; 20,000 cells/well; MOI:0.02; PFU/well:400; Virus volume/well (μL):0.04; Vol for 1500 wells (μL):58.54; Complete with medium (μL): 149941.46; 2) RSV B1 BPR-348-00 (BEI NR-4052), P2 (16-Nov-21); Titre (PFU/mL):3.25E+07; 20,000 cells/well; MOI:0.02; PFU/well:400; Virus volume/well (μL):0.01; Vol for 1500 wells (μL):18.46; Complete with medium (μL):149981.54; 3) Human Metapneumovirus-GFP CAN97-83 (Vira Tree M121); P2 (27 Sep. 2021); Titer (PFU/mL): 3.30E+05; # of cells/well: 20,000; MOI: 0.02; PFU/well: 400; Virus volume/well (μL): 1.21; Vol. for 1500 well (μL): 1818.18; Complete with medium (μL): 14181.82; 4) Human metapneumovirus-TN/96-213 (BEI NR-22243); P2 (27 Sep. 2021); Titer (PFU/mL); 3.35E+05; # of cells/well: 20,000; MOI: 0.02; PFU/well: 400; Virus volume/well (μL): 1.19; Vol. for 1500 wells (μL): 1791.04; Complete with medium (μL): 148208.96.; Using the multichannel pipette, transfer 20 μL of the Ab dilutions to the plated virus; Add 180 μL of the virus for each virus dilution on 96-well plates according to the plate above; Incubate for 30 mins at 37° C.; Aspirate the medium from the cells and transfer 90 μL of the virus/ab complex to the cells; Aspirate the virus and add 100 uL/well of 2.4% Microcrystalline cellulose resuspended in the respective culture medium for each cell line; Incubate for 48h in growth conditions; —day 3: Aspirate the supernatant from the cells with vacuset; Fix the cells with 4% PFA for 30 mins in PFA at room temp; Wash two times with PBS; Add 50 μl/well of primary antibody diluted 1:1,000 for RSV and 1:20 for the MPV in PBS containing 2% BSA and 0.05% Triton X-100; Incubate for 1h at room temp; Wash 3× with PBS; Add secondary anti-mouse AF647 antibody diluted 1:1,000 and nuclei dye Hoescht 1:1,000 in the same buffer as primary antibody; Incubate for 1h at room temp; Wash 3× with PBS and leave in 100 ul/well of PBS; Continue with quantification of infected cells in the Cytation5 plate reader.

Results are presented in Table 9. The 16 mAbs neutralized different RSV and MPV D280 strains with IC50 values ranging from 1.1 to 16.2 ng/ml.

TABLE 9
RSV and MPV Neutralization (IC50 ng/ml)

| mAb | RSV A2 | RSV B1 | MPV A2 | MPV B2 |
|---|---|---|---|---|
| MPK9 | 3.681 | 2.642 | 47.11 | 57.44 |
| MPK10 | 16.18 | 11.79 | 30.59 | 94.92 |
| MPK18 | 2.067 | 3.426 | 69.63 | 70.25 |
| MPK30-v1 | 7.464 | 5.997 | 66.5 | 79.8 |
| MPK36-v3 | 5.56 | 2.16 | 86.33 | 136.5 |
| MPK51 | 1.862 | 1.892 | 53.74 | 50.64 |
| MPK67 | 3.812 | 3.803 | 83.89 | 71.54 |
| MPK73 | 1.094 | 4.613 | 31.74 | 18.55 |
| MPK77 | 1.33 | 2.334 | 29.37 | 30.47 |
| MPK86 | 2.967 | 7.029 | 83.02 | 116.5 |
| MPK92 | 1.581 | 3.054 | 92 | 93.64 |
| MPK99 | 6.106 | 4.712 | 60.12 | 107.6 |
| MPK102 | 4.331 | 5.308 | 95.48 | 123.9 |
| MPK104 | 1.319 | 6.033 | 21.47 | 75.44 |
| MPK108 | 1.502 | 4.564 | 65.35 | 102.2 |
| MPH12 | 12.52 | 7.86 | 112.7 | 74.44 |
| MPE8 | 6.01 | 3.705 | 12.62 | 23.61 |
| MEDI8897 | 0.2081 | 0.4468 | No neutr. | No neutr. |

### Example 6

### Binding Affinity of Antibodies to RSV-F, MPV-F D280, and MPV-F N280

Binding affinity of mAbs for RSV-F protein, MPV-F D280 protein, and MPV-F N280 F variant protein was assessed using surface plasmon resonance (SPR).

### Experiment 1

In further detail, the following settings were used:

Materials— Kinetics Buffer (KB) lx no tween (BSA 0.01% in PBS, pH 7.1); SA-sensors

Conditions— SAX-sensors; pH 7.1, 1000 rpm, 30° C.; volume: 200 ul/well (more volume for a long assay); Sensor Hydratation: >10 min in KB; Loading of F-proteins in KB, 5 min, concentrations were: 0.5 ug/ml for MPV-F N280 (reference), 0.42 ug/ml for MPV-F wt (non-D280N), 0.36 ug/ml for RSV-F wt; Association of the mAb: starting 5 μg/ml in KB, 4 min; Dissociation of mAb: 4 min in KB; Baselines: in KB, 30 sec.

Procedure—Pre-hydrate sensors in KB (see conditions) for at least 10 min; In the meanwhile prepare plate as follows: Column 1: KB; Column 2: RSV-F; Column 3: MPV-F wt (non-D280N); Column 4: MPV-F N280; Column 5: KB; Column 6: MPK series 1; Column 7: MPK series 2; Column 8: MPK series 3. Layout is shown in Table 10.

TABLE 10
Layout for SPR Binding Study

| Sensor | Column 6 | Column 7 | Column 8 |
|---|---|---|---|
| A | MPK 92 | MPK 67 | MPK 102 |
| B | MPK 104 | MPK 99 | MPK 108 |
| C | MPK 51 | MPK 77 | MPK 36 |
| D | MPK 73 | MPK 18 | MPK 86 |
| E | MPK 15 | MPK 65 | MPE8 |
| F | MPK 44 | MPK 10 | MPH12 |
| G | MPK 30 | MPK 9 | RSD5 |
| H | - ref | - ref | - ref |

Runs were as follows: RUN1: 1 (baseline)→2 (loading)→5 (baseline)→6 (association)→5 (dissociation); RUN2: 1 (baseline)→2 (loading)→5 (baseline)→7 (association)→5 (dissociation); RUN3: 1 (baseline)→2 (loading)→5 (baseline)→8 (association)→5 (dissociation); RUN4: 1 (baseline)→3 (loading)→5 (baseline)→6 (association)→5 (dissociation); RUN5: 1 (baseline)→3 (loading)→5 (baseline)→7 (association)→5 (dissociation); RUN6: 1 (baseline)→3 (loading)→5 (baseline)→8 (association)→5 (dissociation); RUN7: 1 (baseline)→4 (loading)→5 (baseline)→6 (association)→5 (dissociation); RUN8: 1 (baseline)→4 (loading)→5 (baseline)→7 (association)→5 (dissociation); RUN9: 1 (baseline)→4 (loading)→5 (baseline)→8 (association)→5 (dissociation)

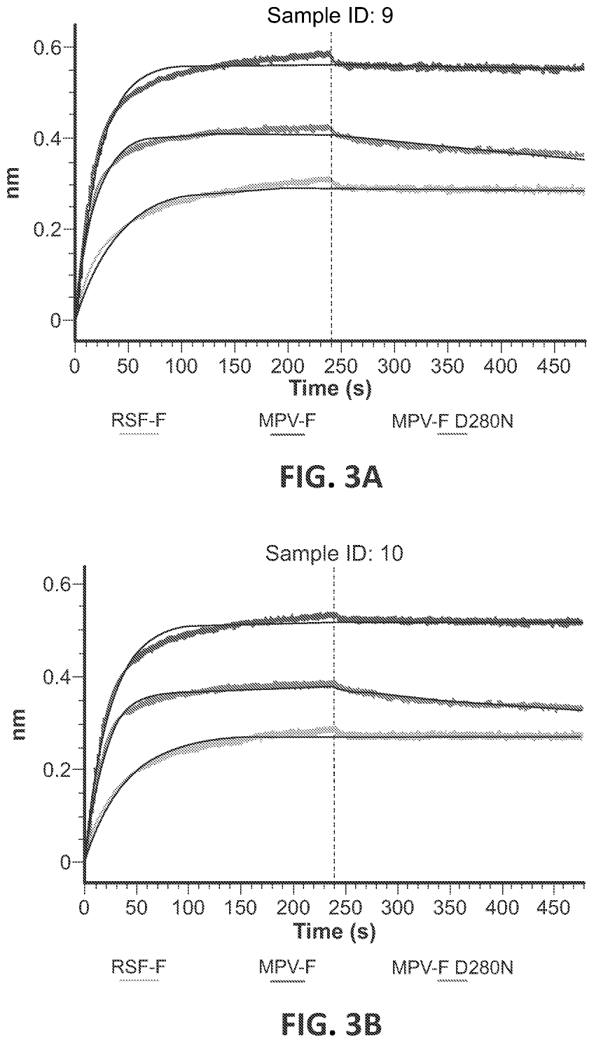

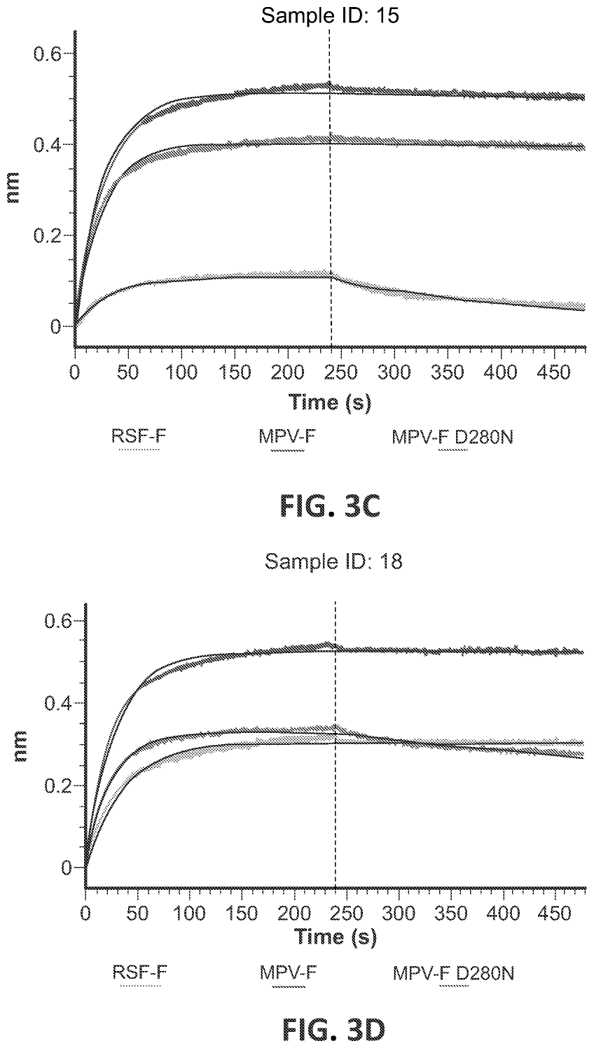

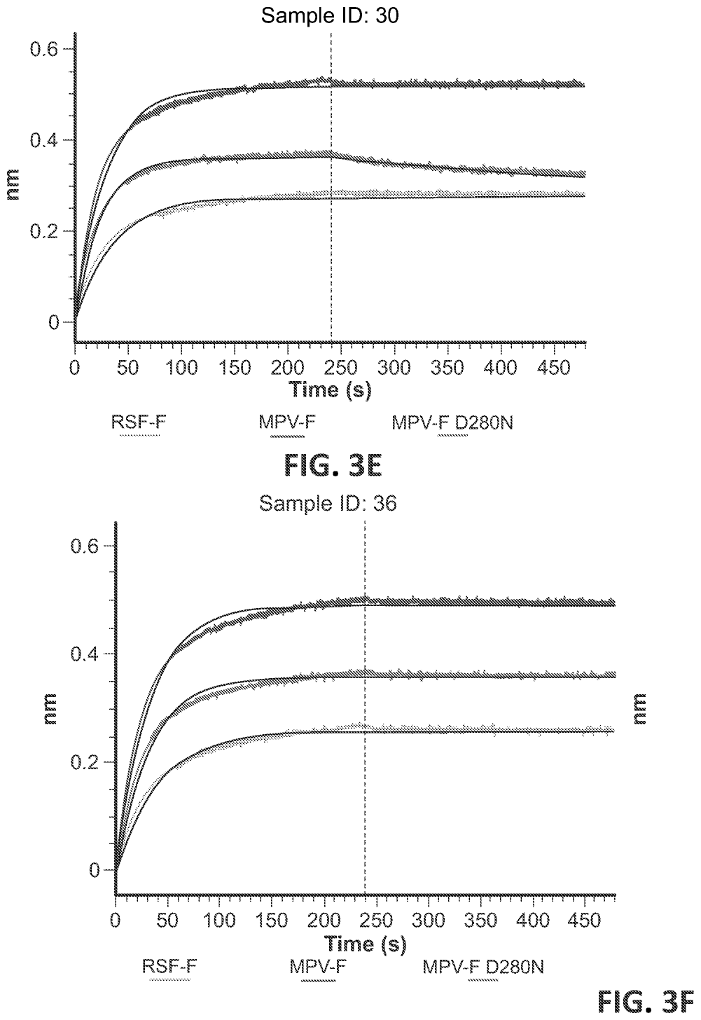

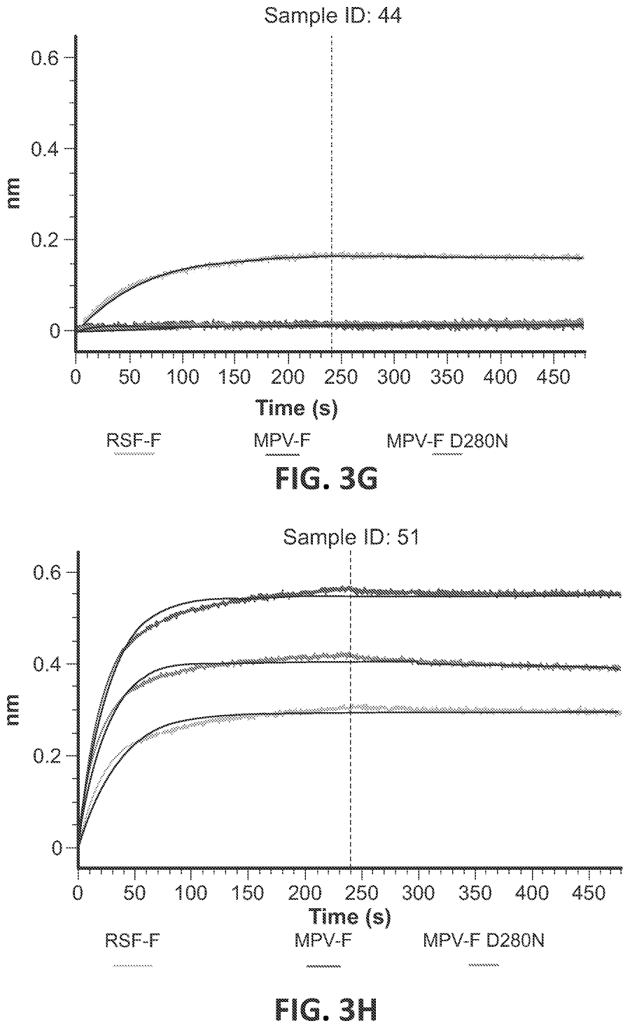

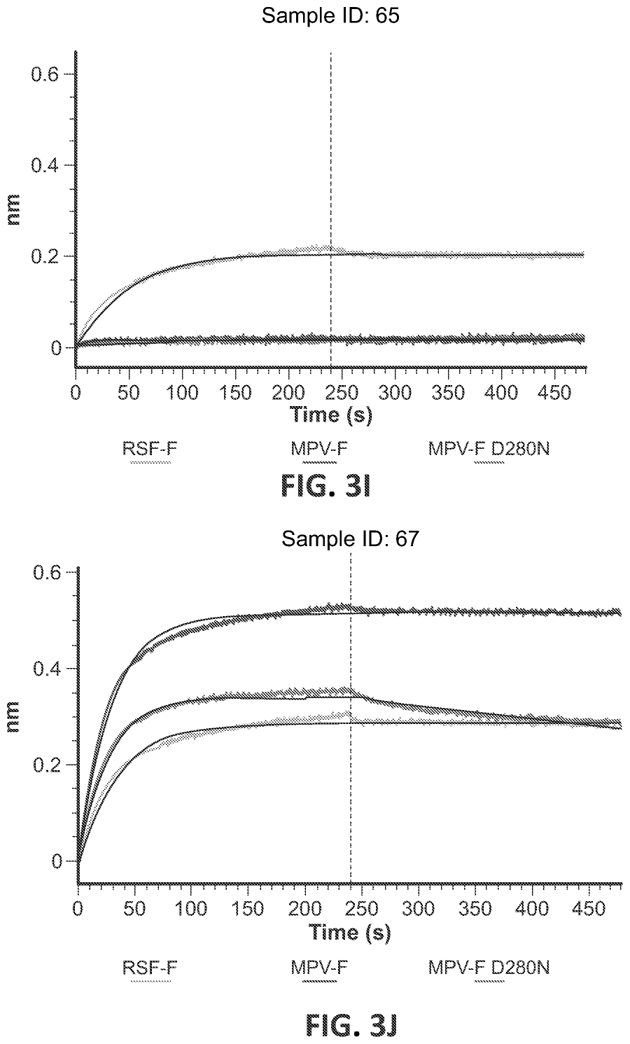

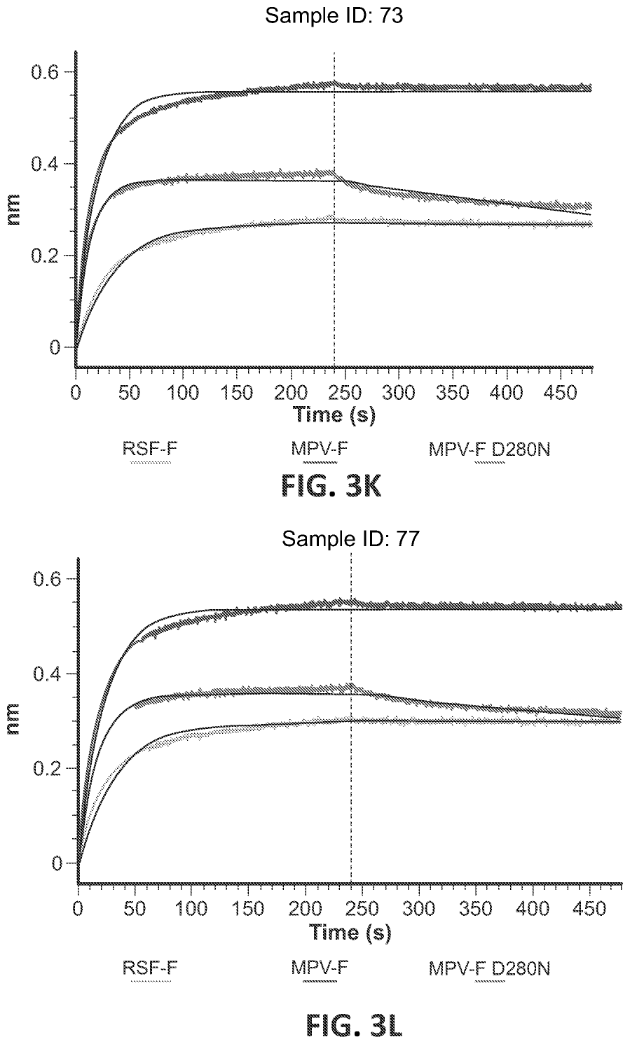

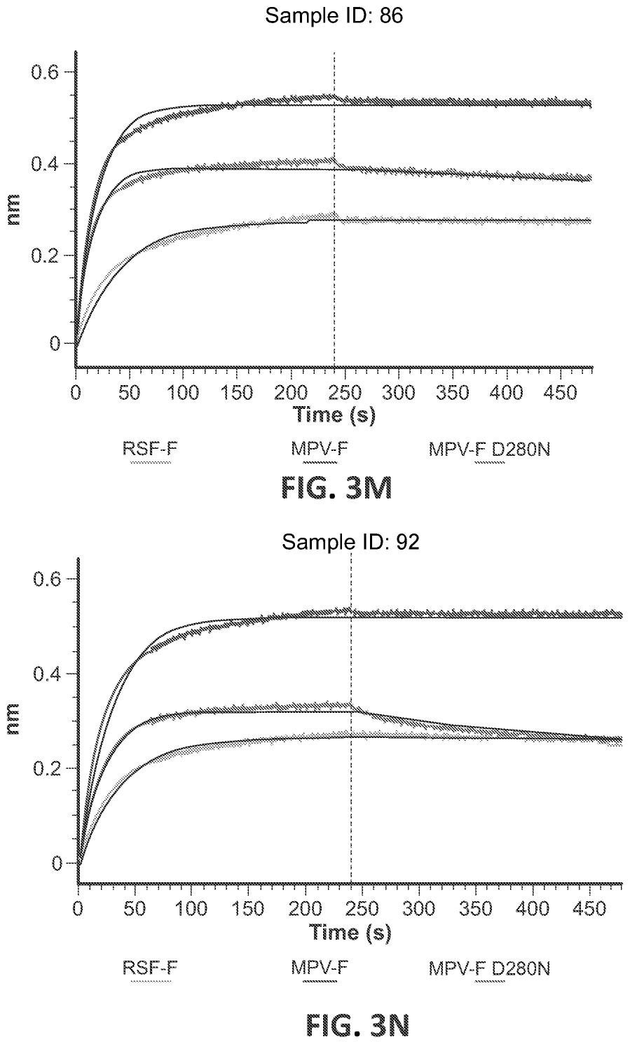

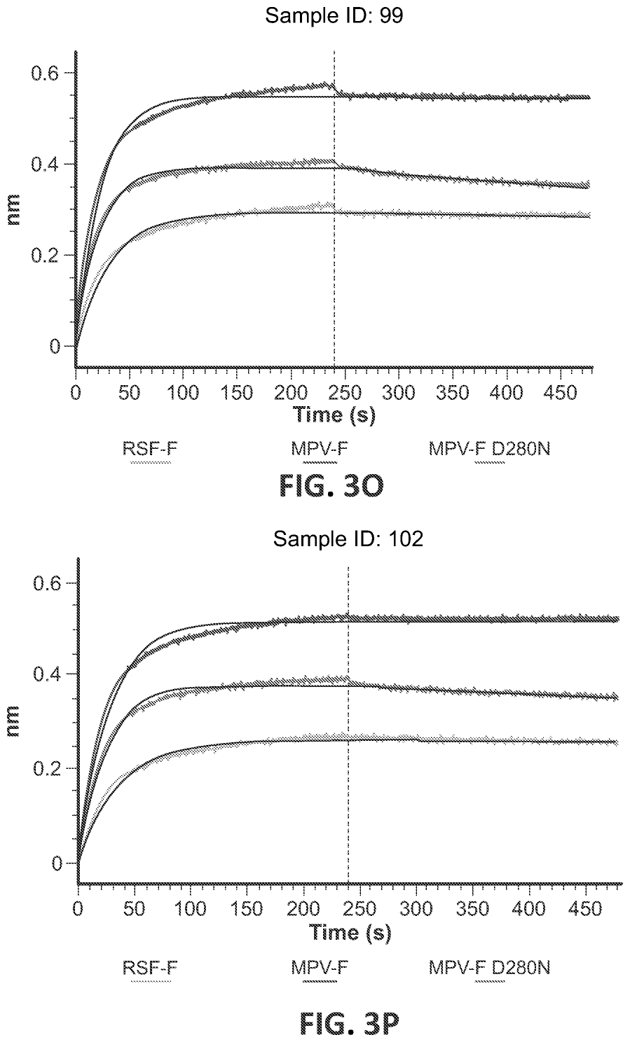

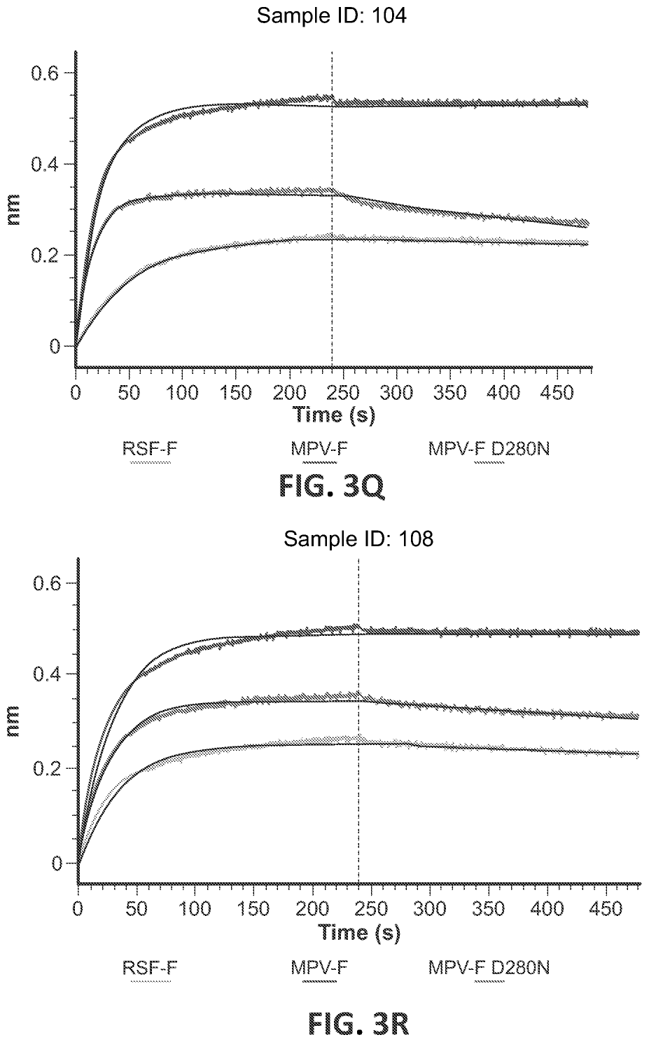

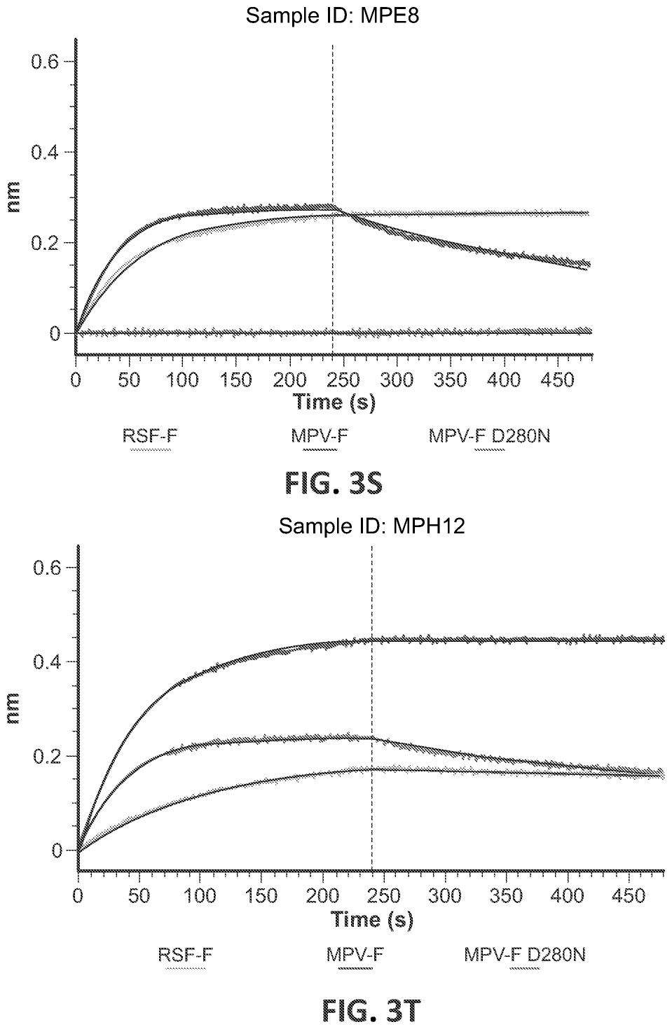

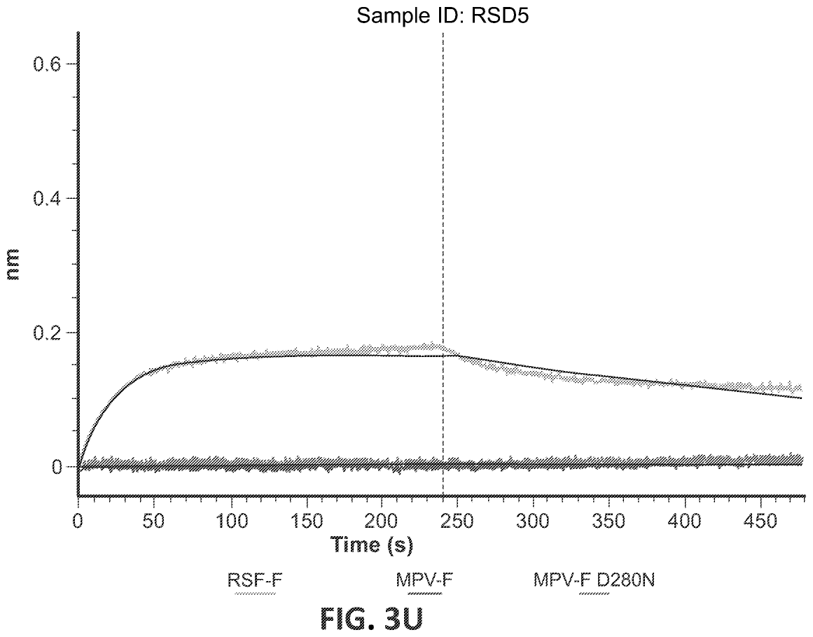

Results are shown and summarized in FIGS. 3A-3U and Table 11. Sample ID at the top of each figure correlates with antibody name (e.g. sample 9 is MPK9, sample 44 is MPK44). MPK36-v3 is highlighted by a red box in FIG. 3F and showed the highest affinity to RSV-F, MPV-F non-D280-N and MPV-F N280. Table 11 summarizes KD values (reported as M). All the mAbs, except for MPK15, showed high-affinity binding to RSV-F. All the MPV-specific mAbs showed high-affinity binding to MPV-F D280 and to MPV-F N280. One mAb, MPK36-v3 showed the highest affinity to RSV-F, MPV-F D280 and MPV-F N280 (1.00E−12 M).

TABLE 11
Antibody KD Values for RSV and MPV

| | KD (M) | | |
|---|---|---|---|
| Sample | RSV-F | MPV-F (non-D280N) | MPV-F N280 |
| MPK9 | 2.01E−10 | 6.49E−11 | 3.56E−10 |
| MPK10 | 1.07E−11* | 1.00E−12* | 3.36E−10 |
| MPK15 | 6.10E−09*** | 7.84E−11 | 5.74E−11** |
| MPK18 | 1.00E−12* | 1.00E−12* | 7.04E−10 |
| MPK30 | 1.00E−12* | 1.00E−12* | 4.33E−10 |
| MPK36-v3 | 1.00E−12* | 1.00E−12* | 1.00E−12* |
| MPK44 | 1.39E−10 | No binding*** | No binding*** |
| MPK51 | 1.00E−12* | 1.00E−12* | 1.30E−10 |
| MPK65 | 2.10E−10 | No binding*** | No binding*** |
| MPK67 | 3.47E−11** | 1.00E−12* | 7.69E−10 |
| MPK73 | 2.69E−11** | 1.00E−12* | 4.24E−10 |
| MPK77 | 1.00E−12* | 1.00E−12* | 3.97E−10 |
| MPK86 | 1.00E−12* | 1.00E−12* | 1.68E−10 |
| MPK92 | 4.38E−11** | 1.00E−12* | 8.36E−10 |
| MPK99 | 1.46E−10 | 4.11E−11** | 3.73E−10 |
| MPK102 | 6.9E−10 | 1.00E−12* | 2.70E−10 |
| MPK104 | 2.82E−10 | 1.00E−12* | 5.49E−10 |
| MPK108 | 4.88E−10 | 1.00E−12* | 4.24E−10 |
| MPE8 | 1.00E−12* | 3.61E−09*** | No binding*** |
| MPH12 | 1.42E−09 | 1.00E−12* | 2.22E−09 |
| RSD5 | 1.71E−09 | No binding*** | No binding*** |

No indicator = low affinity;
*= high affinity;
**medium affinity;
***= not measurable/no binding

### Experiment 2

An experiment conducted in the same manner as Experiment 1 using Run1 protocol provided the additional RSV results provided in Table 12. All mAbs showed high-affinity binding to RSV-F (KD ranging from 2.8E−9 to 1.0E−12 M), with 2 mAbs (22%) showing very stable binding with little dissociation (KD<1.0E−11).

TABLE 12
Antibody KD Values for RSV

| mAb | KD RSV (M) | mAb | KD RSV (M) |
|---|---|---|---|
| MPK44 | 2.6E−10 | MPK169-v2 | 6.2E−10 |
| MPK167 | 1.1E−11 | MPK171-v1 | 1.2E−09 |
| MPK177 | 1.0E−12 | MPK65-v2 | 1.4E−11 |
| MPK182 | 2.8E−09 | RSD5 | 2.3E−09 |
| MPK191 | 2.9E−11 | MEDI8897 | 1.0E−12 |
| MPK195 | 1.0E−12 | palivizumab | 2.6E−09 |

### Experiment 3

An experiment conducted in the same manner as Experiment 1, but with the plate prepared as follows: Column 1: KB, Column 3: MPV-F D280, Column 4: MPV-F N280, Column 5: KB, Column 6: MPK15, and run according to RUN10: 1 (baseline)→3/4 (loading)→5 (baseline)→6 (association)→5 (dissociation) provided the results in Table 13. MPK15 showed high-affinity and similar binding to MPV-F D280 and MPV-F N280 (KD: 7.1E−11 M and 7.3E−11 M, respectively). MPK15 bound with higher affinity compared to MPE33 and MPF5.

TABLE 13
MPK15 KD Values for MPV

| mAb | KD MPV (M) | KD D280N(M) |
|---|---|---|
| MPK15 | 7.1E−11 | 7.2E−11 |
| MPE33 | 9.1E−10 | 1.2E−09 |
| MPF5 | 1.3E−10 | 1.3E−10 |

### Experiment 4

An experiment conducted in the same manner as Experiment 1 using the RUN1 protocol, but with the plate prepared as follows: Column 1: KB, Column 2: RSV-F, Column 3: MPV-F D280, Column 4: MPV-F N280, Column 5: KB, Column 6: MPK series 1, Column 7: MPK series 2, Column 8: MPK series 3, provided the results presented in Table 14. Most mAbs (37, 90%) showed high-affinity binding to RSV-F (KD ranging from 1.0E−9 to 1.0E−12 M), with 21 mAbs (51%) showing very stable binding with little dissociation (KD<1.0E−11). Most mAbs (37, 90%) showed high-affinity binding to MPV-F D280 (KD ranging from 1.0E−9 to 1.0E−12 M), with 33 mAbs (88%) showing very stable binding with little dissociation (KD<1.0E−11). Most mAbs (26, 63%) showed high-affinity binding to MPV-F N280 mutant (KD ranging from 1.0E−9 to 1.0E−12 M), with 2 mAbs (5%) showing very stable binding with little dissociation (KD<1.0E−11). One mAb, MPK36-v3, showed the highest affinity to RSV-F, MPV-F D280 and MPV-F N280 (about 1.0E−12 M).

TABLE 14
Antibody KD Values for RSV and MPV

| | | KD MPV F | KD MPV F |
|---|---|---|---|
| mAb | KD RSV F (M) | Non-D280N (M) | D280N (M) |
| MPK9 | 4.0E−11 | 8.1E−12 | 3.5E−10 |
| MPK10 | 1.2E−11 | 1.0E−12 | 3.8E−10 |
| MPK18 | 1.0E−12 | 1.0E−12 | 7.8E−10 |
| MPK30-v1 | 1.0E−11 | 1.0E−12 | 4.5E−10 |
| MPK36-v3 | 5.6E−12 | 1.0E−12 | 1.0E−12 |
| MPK51 | 1.0E−12 | 1.0E−12 | 1.2E−10 |
| MPK67 | 5.9E−12 | 1.0E−12 | 8.6E−10 |
| MPK73 | 1.2E−11 | 1.0E−12 | 6.5E−10 |
| MPK77 | 1.0E−12 | 1.0E−12 | 6.8E−10 |
| MPK86 | 1.0E−12 | 1.0E−12 | 1.2E−10 |
| MPK92 | 9.8E−11 | 1.0E−12 | 9.5E−10 |
| MPK99 | 1.5E−10 | 4.1E−11 | 3.7E−10 |
| MPK102 | 9.6E−11 | 1.0E−12 | 2.8E−10 |
| MPK104 | 7.9E−11 | 1.0E−12 | 6.3E−10 |
| MPK108 | 5.8E−10 | 1.0E−12 | 4.5E−10 |
| MPK126 | 1.0E−12 | 1.0E−12 | 2.1E−10 |
| MPK127 | 1.7E−09 | 1.0E−12 | 1.0E−12 |
| MPK129 | 8.3E−11 | 1.0E−12 | 1.9E−09 |
| MPK130 | 1.6E−11 | 1.8E−11 | 1.9E−09 |
| MPK132-v1 | 4.7E−10 | 1.0E−12 | 4.3E−09 |
| MPK133 | 1.0E−12 | 8.2E−11 | 3.7E−09 |
| MPK136 | 1.0E−12 | 1.0E−12 | 4.6E−09 |
| MPK141 | 1.3E−10 | 1.0E−12 | 8.4E−10 |
| MPK142-v1 | 1.0E−07 | 1.0E−07 | 1.0E−07 |
| MPK142-v2 | 1.6E−08 | 2.7E−12 | 2.0E−10 |
| MPK144 | 1.0E−12 | 8.1E−10 | 1.0E−07 |
| MPK145 | 1.0E−12 | 1.0E−12 | 8.5E−10 |
| MPK146 | 9.5E−12 | 1.0E−12 | 7.2E−09 |
| MPK149 | 1.0E−12 | 1.0E−12 | 4.0E−10 |
| MPK150-v2 | 1.0E−12 | 1.0E−12 | 5.8E−10 |
| MPK151 | 8.0E−11 | 1.0E−12 | 2.7E−10 |
| MPK152 | 1.0E−12 | 1.0E−12 | 6.9E−11 |
| MPK153 | 4.6E−10 | 1.0E−12 | 5.5E−09 |
| MPK155 | 6.3E−12 | 1.0E−12 | 2.6E−09 |
| MPK157 | 1.0E−12 | 1.0E−12 | 1.2E−09 |
| MPK158 | 1.0E−12 | 1.0E−12 | 2.9E−10 |
| MPK162 | 1.0E−07 | 1.0E−07 | 1.0E−07 |
| MPK174-v2 | 1.0E−12 | 1.1E−08 | 1.0E−07 |
| MPK190 | 1.0E−12 | 1.0E−12 | 5.7E−10 |
| MPK196 | 1.0E−12 | 3.8E−09 | 1.0E−07 |
| MPK204 | 2.9E−10 | 1.0E−12 | 2.6E−09 |
| MPH12 | 7.5E−10 | 1.0E−12 | 1.7E−09 |
| MPE8 | 1.0E−12 | 2.5E−09 | 1.0E−07 |
| MEDI8897 | 1.0E−12 | 1.0E−07 | 1.0E−07 |
| palivizumab | 2.6E−09 | 1.0E−07 | 1.0E−07 |

### Experiment 5

MPK73-v1, MPK65-v2, MPK44, MPK36v3, MPK15, MPH12, RSV/MPV cross-neutralizing comparator antibody MPE8-v3 (an V-region-engineered variant of MPE8), and anti-RSV-F comparator antibodies D25 (see Kwakkenbos et al., Nat Med 16(1):123-128 (2009); doi: 10.1038/23.2071), and RSD5-FR-GL (a germline-reverted version of RSD5, in which somatic mutations in the framework regions were reverted to germline; Jones et al. PLoS Patho. 15(7):e1007944 (2019); doi:10.1371/journal.ppat.1007944)) were evaluated in a competitive binding assay using surface plasmon resonance (SPR) and RSV-F. All antibodies were expressed as recombinant human IgG1 and, except for D25 and RSD5-FR-GL, included M428L and N434S mutations (“MLNS”) in the Fc.

In further detail, the following settings were used:

Materials: Kinetics Buffer 1× no tween: 0.01% BSA in PBS; SAX sensors (see related products).

Conditions: SAX sensor; 384 well plate; volume: 70 ul/well; pH 7.1, 1000 rpm, 30° C.; Sensor Hydratation: >10 min in KB; Loading Antigen: 0.5 pg/ml, 8 min in KB; Association of mAbs: 15 μg/ml in KB, 7 min; Baselines: in KB, 30 sec.

Procedure: pre-hydrate sensors in KB (see conditions) for at least 10 min; In the meanwhile prepare plate for loading with antibodies having the concentrations set forth in Table 15. Perform RUN: Baseline in A1→Loading in A2→Baseline in A1→Association 1 in A3→Association 2 in A4.

TABLE 15
Run Loading Values for RSV/MPV KD Assay

| mAb | mg/ml | mAb | mg/ml |
|---|---|---|---|
| MPK73-v1-rIgG1-LS | 1.036 | MPH12-rIgG1-LS | 4.003 |
| MPK65-v2-rIgG1-LS | 2.564 | MPE8-ve-rIgG1-LS | 3.857 |
| MPK44-rIgG1-LS | 1.543 | D25-rIgG1 | 2.48 |
| MPK36-v3-rIgG1-LS | 2.858 | RSD5-FR-GL-rIgG1 | 3.5 |
| MPK15-rIgG1-LS | 1.008 | | |

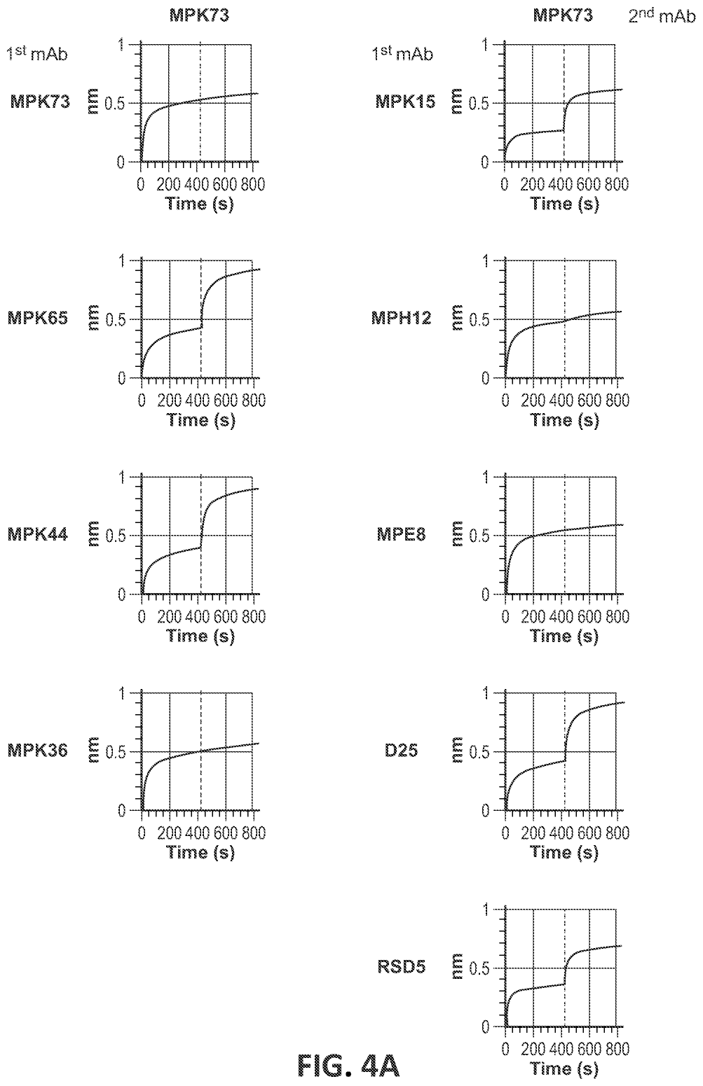

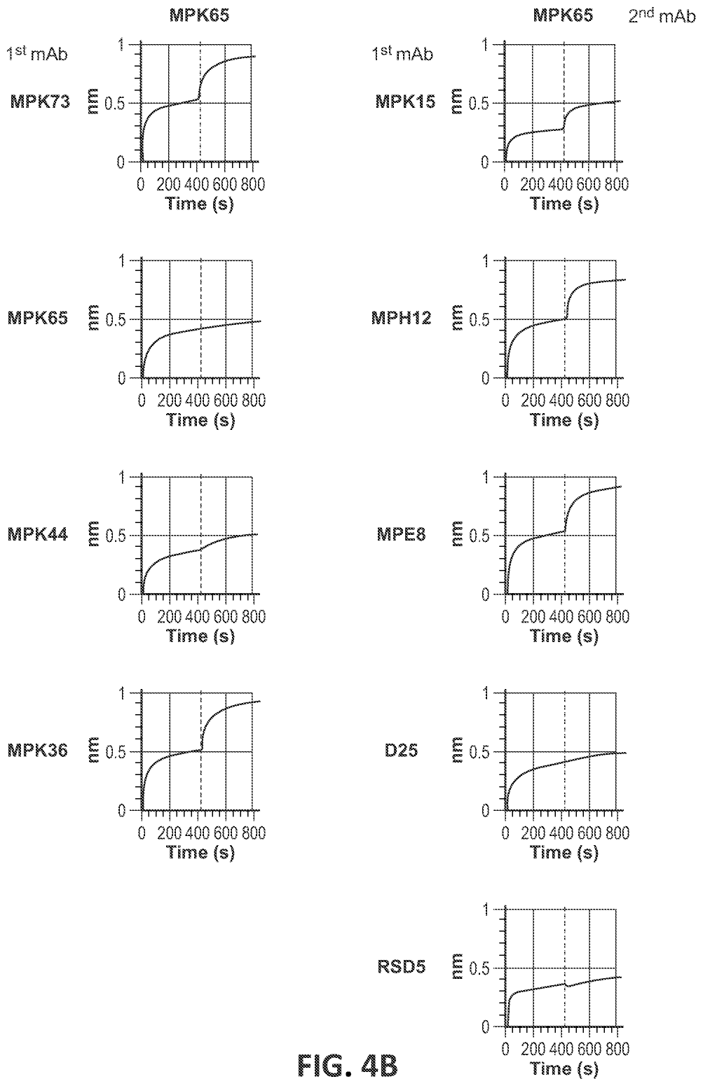

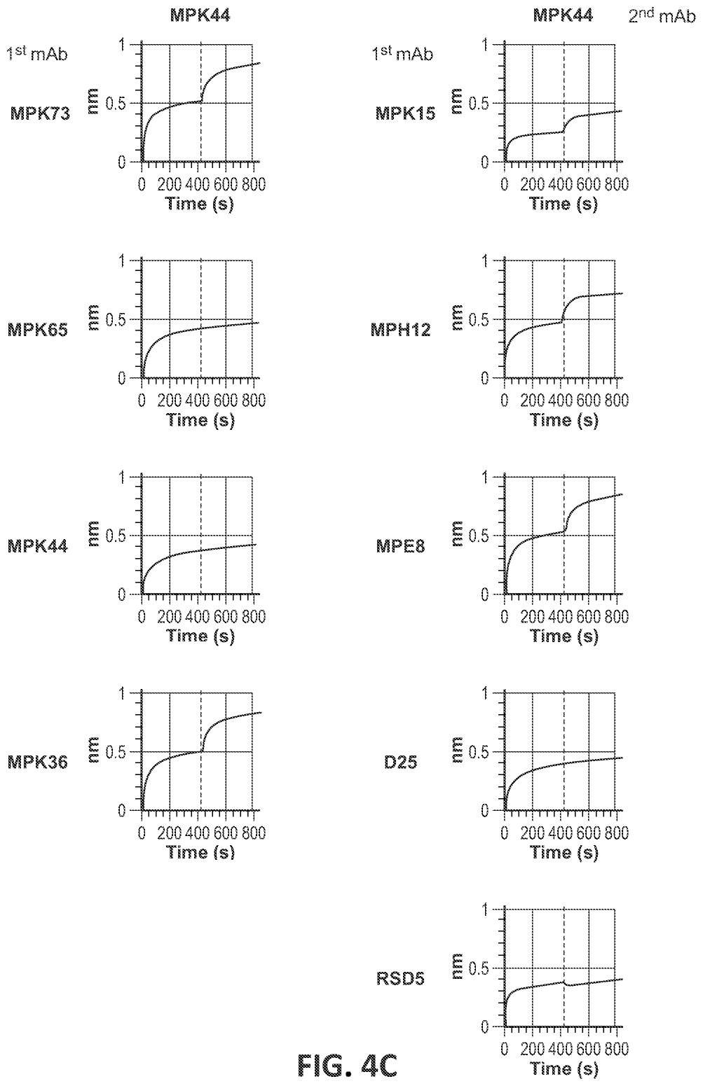

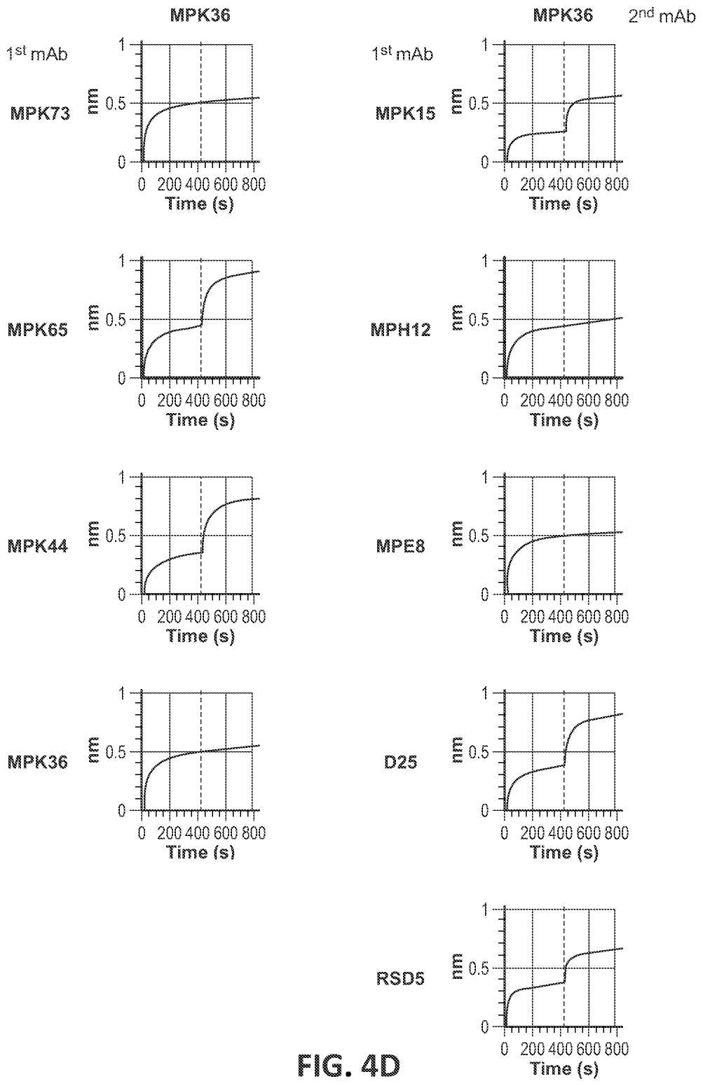

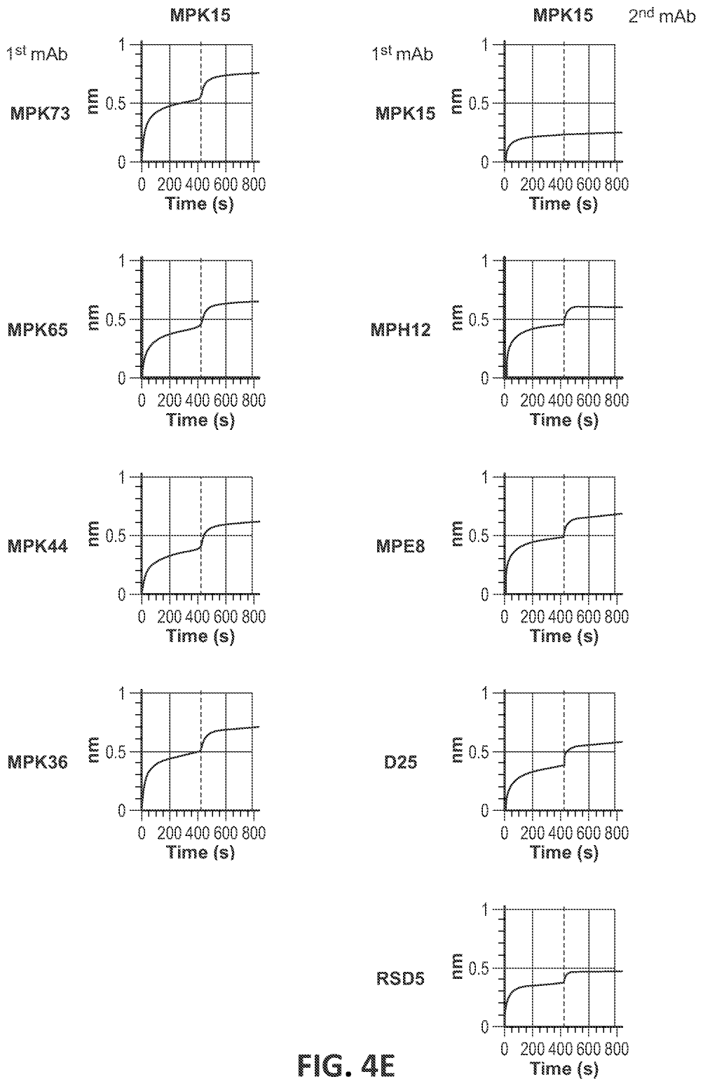

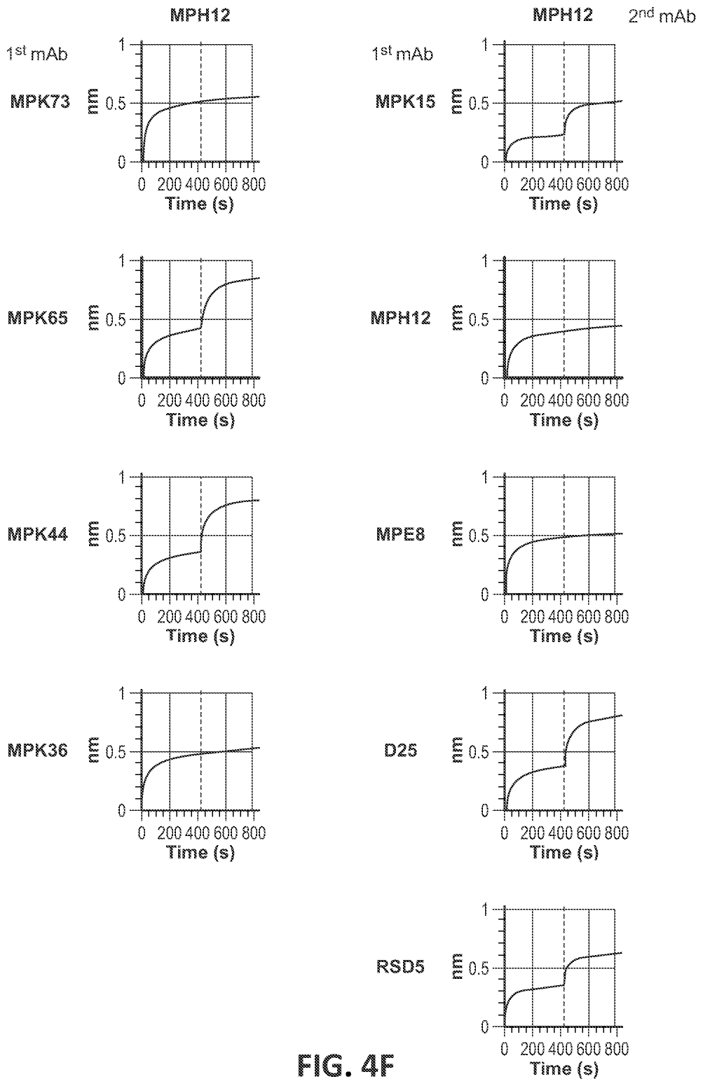

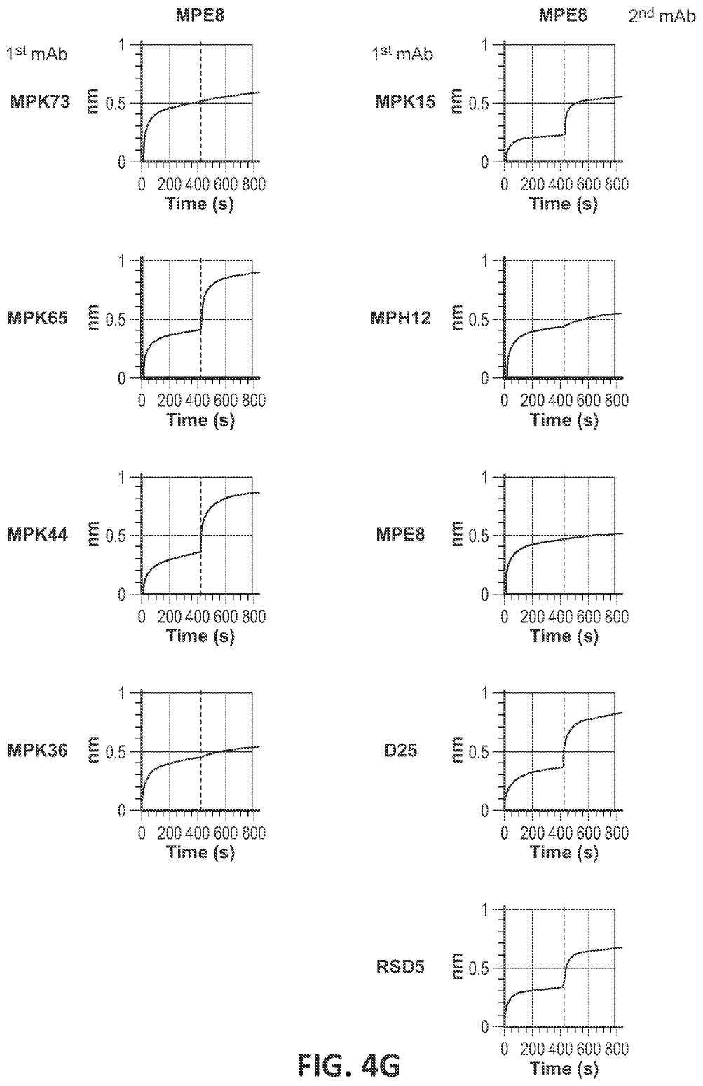

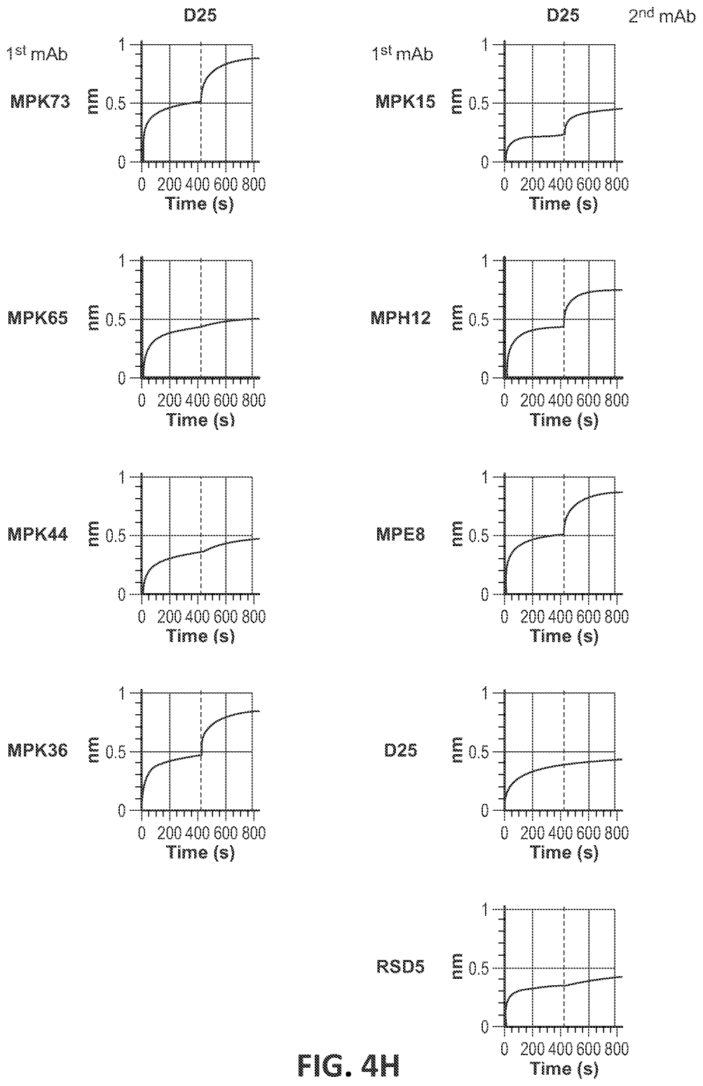

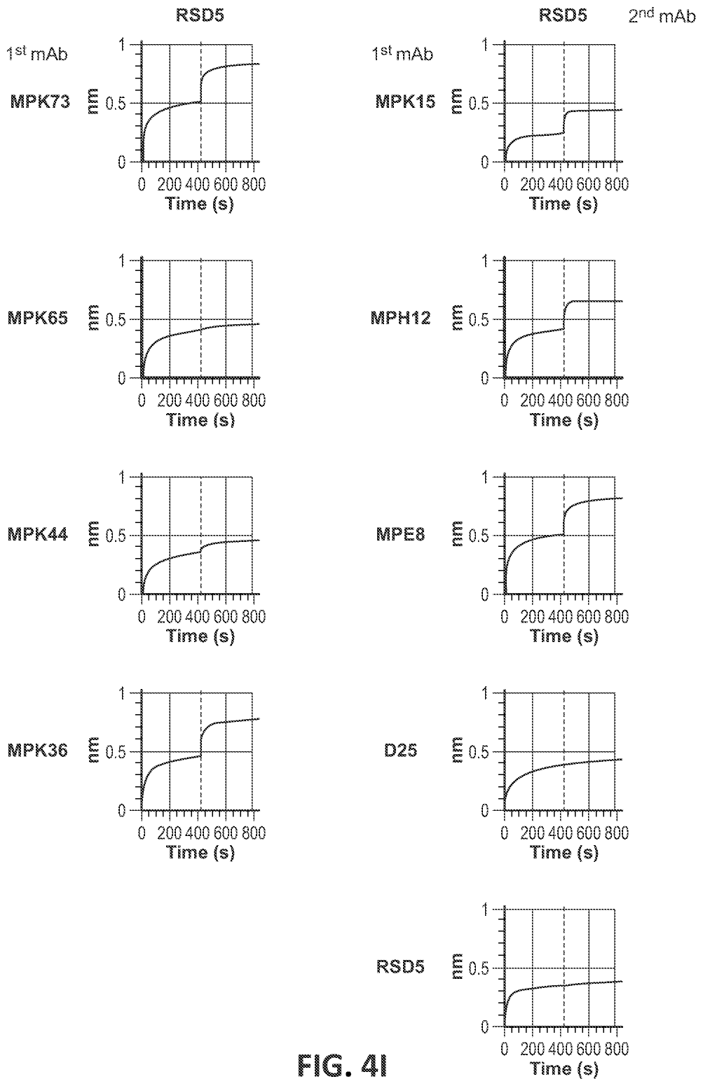

FIGS. 4A-4I show surface plasmon resonance data for each antibody pair tested. MPH12, MPE8, MPK36 and MPK73 competed with one another, suggesting that they recognize the same epitope. MPK65 and MPK44 competed with one another and with D25 and RSD25, suggesting that they recognize the same epitope. MPK15 did not compete with any mAb, suggesting that it recognizes a different epitope.

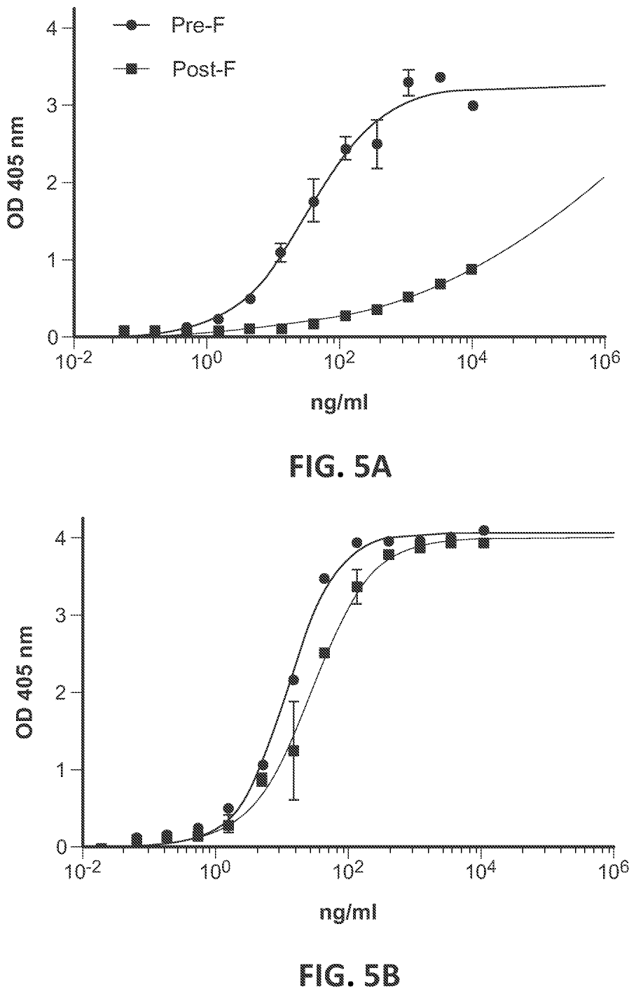

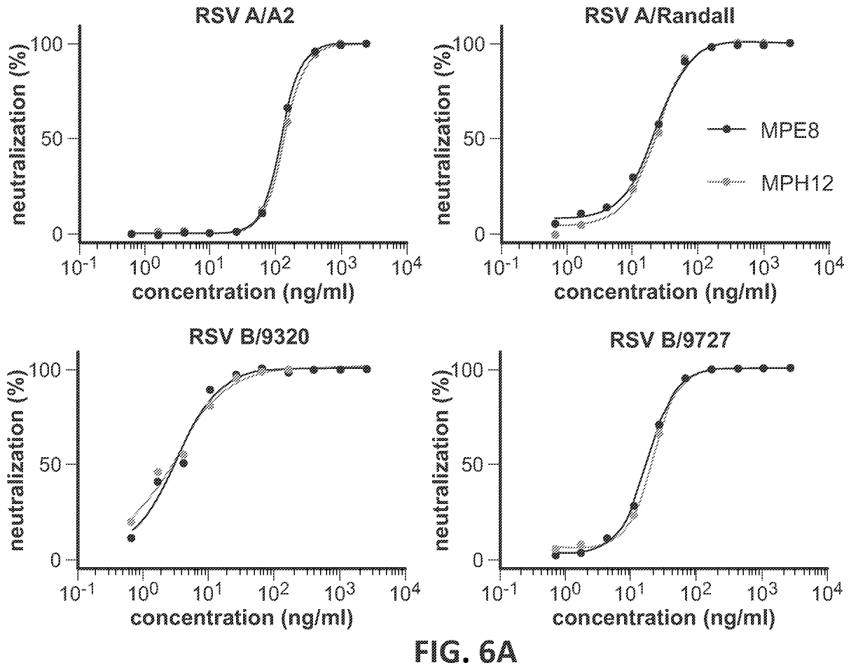

In a separate set of experiments, the ability of MPE8 and MPH12 to bind to post-fusion RSV F protein was assayed. Results are presented in FIG. 5A and FIG. 5B. Overall, mAbs that bind to site III of RSV F protein appear to be pre-fusion specific.

### Example 7

### Cross-Neutralization of RSV A and B Strains and MPV A and B (Non-D280N) Strains by MPH12

MPH12 was tested for cross-neutralization of RSV (A and B strains) and MPV (A and B strains, non-D280N). MPE8, which cross-neutralizes RSV and MPV, was included as a comparator.

For testing neutralization of RSV A and B strains, a 384-well plate format was used, with microscopy plates from TTP Labtech. Six replicates were prepared. Serial dilutions of mAbs were prepared in growing medium (GM, MEM 10% Hyclone+Penicillin/Streptomycin) and pre-incubated together with virus diluted in infection medium (IM, MEM 1% HYyclone+P/S). Pre-incubation mAb concentration was 2,500 ng·ml final at 1:2.5 serial dilutions. Viral input was 300 TCID50/well or pfu/well: RSV A/Randall, batch 1 (1.92×10⁵ TCID50/ml); RSV A/A2/61, batch 10 (3.12×10⁵ TCID50/ml); RSV B/9727/09, batch 1 (2.4×10⁵ pfu/ml); and RSV B/9320, batch 1 (8.0×10⁴ pfu/ml). Pre-incubation was for 45 min at 37° C. Following pre-incubation, 1000 HEp-2 cells were added at 1000 cells/well. Volumes were 10 ul mAb 4× (in GM)+10 ul virus (in IM)+20 ul cells (in IM) (day4: +10 ul detection solution). Spreading time was 3 days (infection progressed well so detection was anticipated by 1 day). Detection solution: Draq5 5× (1:400→1:2000 final)+Motavizumab-DL488 5× (2 ug/ml→0.4 ug/ml final). Detection solution incubation: 4h.

The protocol was as follows:

day 0: Make serial dilution of the mAb in GM and add 10 ul/well; dilute virus in IM and add 10 ul/well; Incubate 45 min at 37° C.; Add cells (20 ul/well for RSV); Incubate for time needed for the virus to spread; day 3: Add 10 ul Detection solution; Incubate and read at the appropriate time; Plates: RSV A (A2 top, randall bottom); RSV B (9320 top, 9727 bottom); mAbs order: MPE8, MPH12

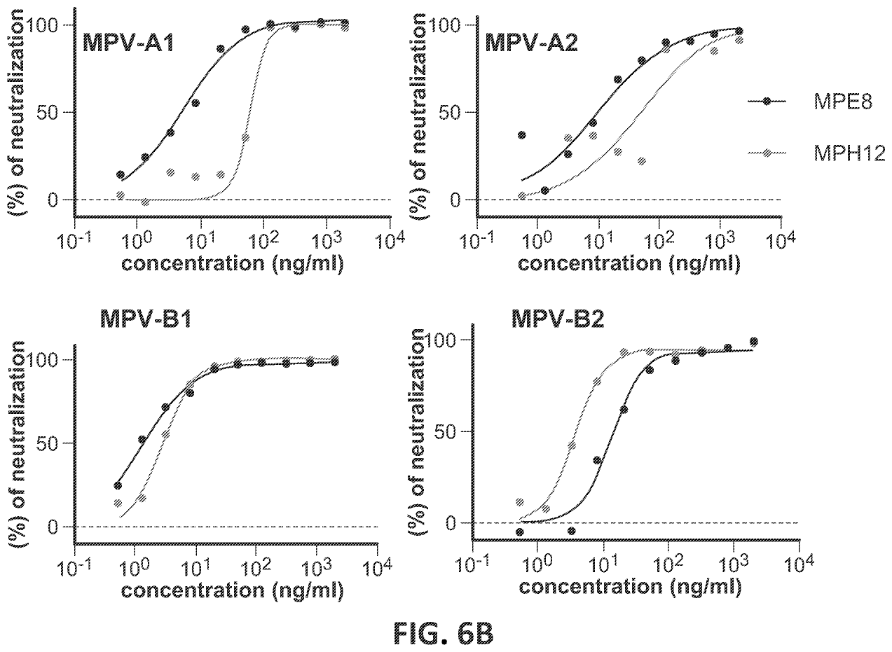

Infection was allowed to spread for a total of three days prior to detecting neutralization at day 3. Results are shown in FIG. 6A and Table 16. MPH12 and MPE8 neutralized RSV strains with similar potency to one another.

TABLE 16
RSV IC50 (ng/ml)

| | A/A2 | A/Randall | B/9320 | B/9727 |
|---|---|---|---|---|
| MPE8 | 125.9 | 22.04 | 3.196 | 16.93 |
| MPH12 | 142.1 | 23.08 | 3.285 | 19.89 |

For testing of neutralization against MPV A and B (non-D280N) strains, the following settings were used:

Material— MPV A1/6621 (a MPV D280 strain), batch 15, 1.78×10⁵ TCID50/ml; MPV A2/8908 (a MPV D280 strain) batch 1 (05.04.12): 6.4×10⁴ pfu/ml; MPV B1/4702 (a MPV D280 strain) batch 5 (09.01.12): 3.30×10⁶ TCID50/ml; MPV B2/3817 (a MPV D280 strain) batch 5 (09.01.12): 9.59×10⁴ TCID50/ml

Conditions— Format: 384 wp; microscopy plates from TTP Labtech; replicates: 6 replicates; medium: Infection medium (IM): MEM 1% Hyclone+P/S. growing medium (GM): MEM 10% Hyclone+P/S, serum free medium (SFM): MEM+P/S; Pre-incubation mAb-virus: 45 min at 37° C.; Draq5 final dilution (in 50 ul): 1:2000; mAb concentration: 10,000 ng/ml (→2000 ng/ml final when TPCK trypsin is added)→1:2.5 serial dilutions; virus input: 350 TCID50/well or pfu/well; cells: LLC-MK2, 1200 cells/well; volumes: 10 ul mAb (in GM)+10 ul virus (in IM)+10 ul cells (in IM) (day1: +20 ul Trypsin TPCK (in SFM)) (+10 ul detection solution on reading-day); spreading time: 4 days; Trypsin TPCK: 125 μg/ml in serum free medium→50 μg/ml final; detection solution: Draq5 6× (1:333→1:2000 final)+234-DL488 at 6× (2.4 ug/ml→0.4 ug/ml final); detection solution incubation: 4h

Protocol— day 0: Make serial dilution of the mAb in GM and add 10 ul/well; dilute virus in IM and add 10 ul/well; Incubate 45 min at 37° C.; Add 10 ul/well cells in IM; Incubate for time needed for the virus to spread; —day 1: Add 20 ul/well Trypsin TPCK in SFM to a final concentration of 40 μg/ml; —day 4: Add 10 ul Detection solution; Incubate and read at the appropriate time; Plates sequence: plate 1: MPV/A (A1/6621+A2/8908), plate 2: MPV/B (B1/4702+B2/3817); mAb sequence: MPE8v3, MPH12.

Results are shown in FIG. 6B and Table 17. In this assay, MPE8 neutralized A1, A2, and B1 strains with greater potency than MPH12 (though potency against B1 strain was similar to MPH12), while MPH12 neutralized B2 with greater potency than MPE8.

TABLE 17
MPV D280 IC50 (ng/ml)

| | A1/6621 | A2/8908 | B1/4702 | B2/3817 |
|---|---|---|---|---|
| MPE8 | 5.226 | 9.282 | 1.314 | 13.62 |
| MPH12 | 59.00 | 50.30 | 2.848 | 3.715 |

### Example 8

### Mutagenesis Studies of RSV/MPV Cross-Reactive MAB MPH12

MPH12 carries cysteine residues at positions 111 and 112.2 in VH. Unpaired cysteines present a potential problem for developability and manufacturing. Mutagenesis studies were performed to investigate contribution of these cysteines to antibody structure and function.

Substitution mutations were generated by site-directed mutagenesis at heavy chain positions C111 and C112.2 (in VH, using the IMGT numbering system), generating four MPH12 variants, MPH12v1-MPH12v4, as described in Table 18. Antibodies were generated by transfection in CHO-Expi cells and day 7 production titers were determined by ELISA (Table 18).

TABLE 18
MPH12-v1-v4 and Productivity

| Antibody | Mutation | Production (μg/mL) |
|---|---|---|
| MPH12-v1 | C111S/C112.2S | 2545.3 |
| MPH12-v2 | C111A/C112.2A | 1992.0 |
| MPH12-v3 | C111A/C112.2S | 1613.7 |
| MPH12-v4 | C111S/C112.2A | 1243.1 |

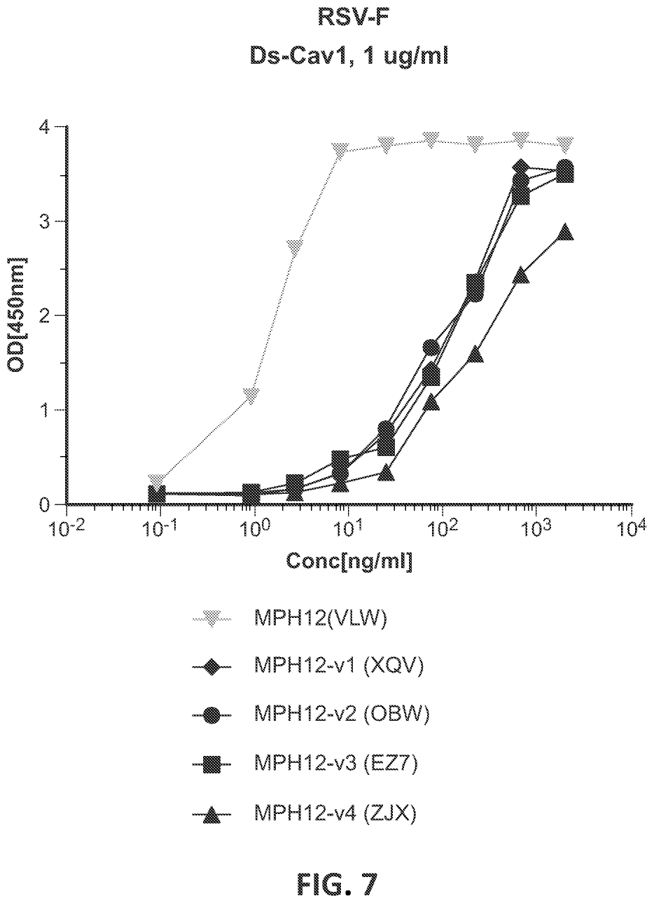

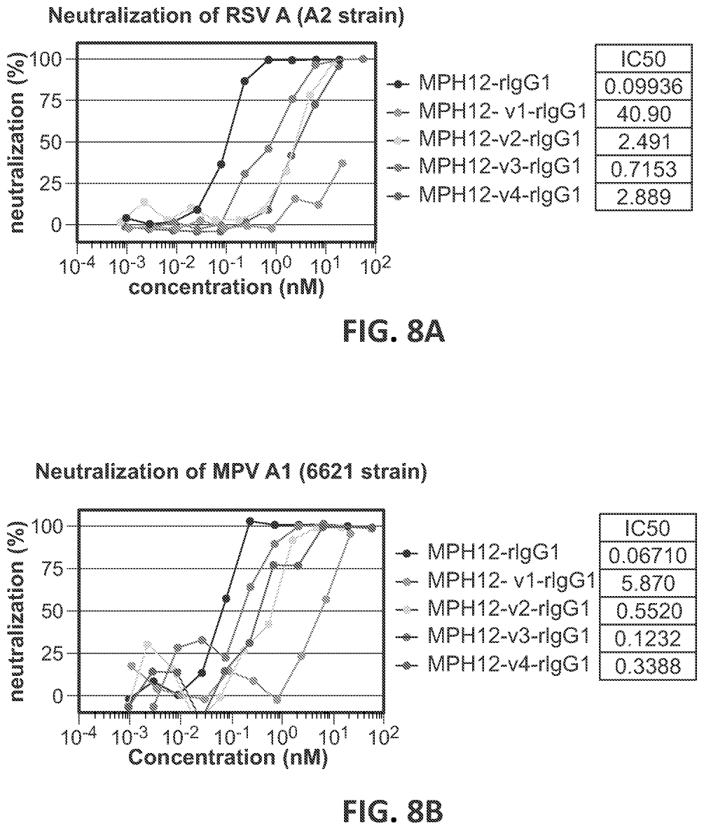

DS-Cav1 RSV F is a modified RSV glycoprotein, wherein disulfide and cavity-filling mutations have been introduced (S155C, S190F, V207L, and S290C) to produce a RSV-F stabilized in the pre-fusion conformation (see Joyce et al. Pathog Immun. 4(2):294-323 (2019); doi: 10.20411/pai.v4i2.338; the DS-Cav1 mutations and DS-Cav10stabilized RSV F of which are incorporated herein by reference). Binding of MPH12v1-MPH12v4 and MPH12 to DS-Cav1 RSV F was measured by ELISA and results are presented in FIG. 7. Neutralization of RSV A/A2/61 (FIG. 8A and Table 19) and MPV A1/6621 (FIG. 8B and Table 19) by MPH12v1-MPH12v4 and MPH12 was also measured. These results showed that VH C111 and C112.2 contribute to binding and neutralization function in MPH12.

TABLE 19
MPH12-v1-v4 RSV and MPV D280 Neutralization

| Antibody | RSV IC50 (nM) | MPV IC50 (nM) |
|---|---|---|
| MPH12 | 0.09936 | 0.06710 |
| MPH12-v1 | 40.90 | 5.870 |
| MPH12-v2 | 2.491 | 0.5520 |
| MPH12-v3 | 0.7153 | 0.1232 |
| MPH12-v4 | 2.889 | 0.3388 |

### Example 9

### Structural Analysis MPH12

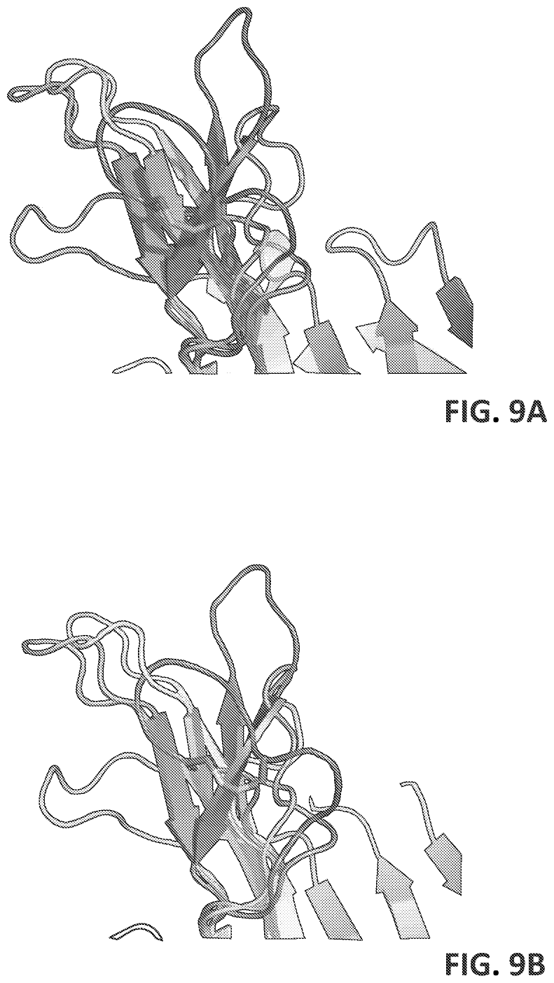

The crystal structure of MPH12 Fab (alone or in complex with RSV-F or MPV-F D280) was determined, and five homology models were generated for comparison to the crystal structure. Rotated views of homology modeling of MPH12 H-CDR3 are shown in FIG. 9A and FIG. 9B, wherein five homology models are shown in various shades of green and the crystal structure of MPH12 is shown in grey. A disulfide bond was not observed in any of the five homology models. Furthermore, the loop conformation determined using the crystal structure was different from that of the predicted models.

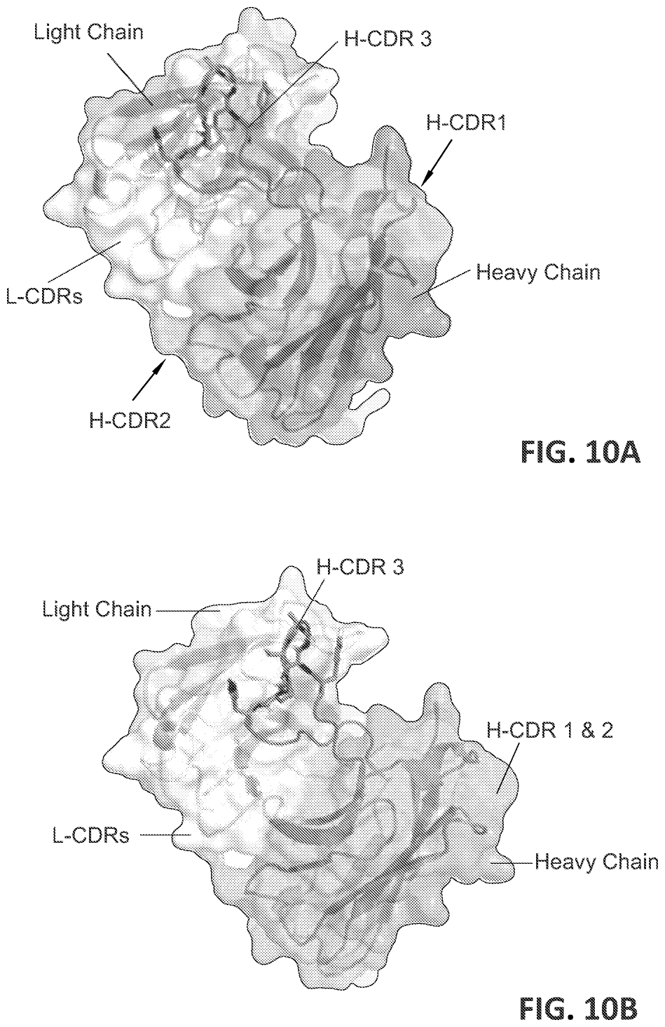

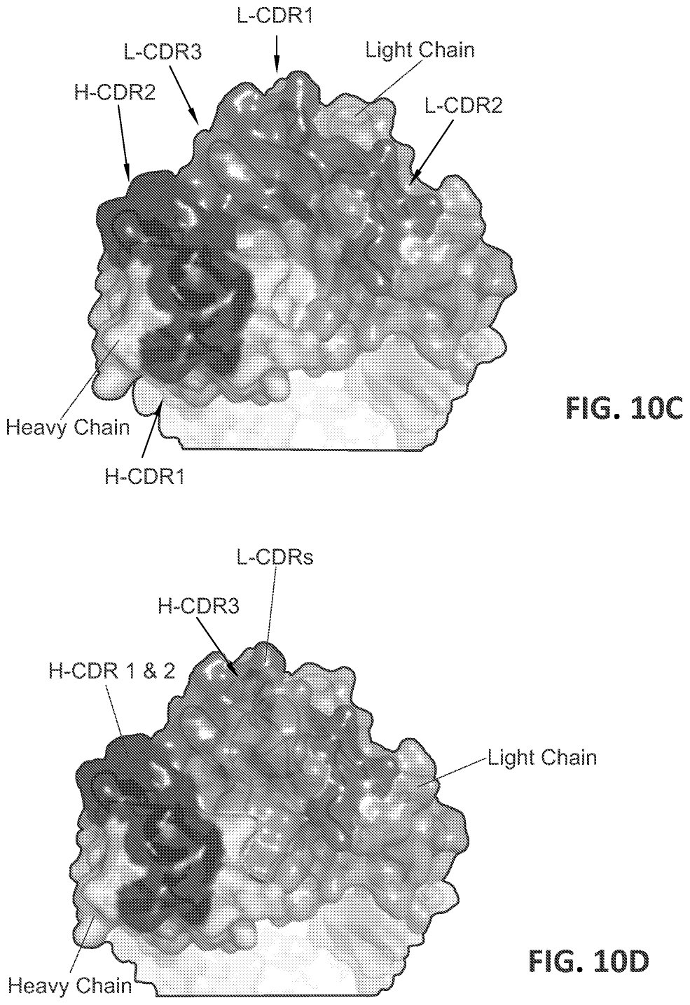

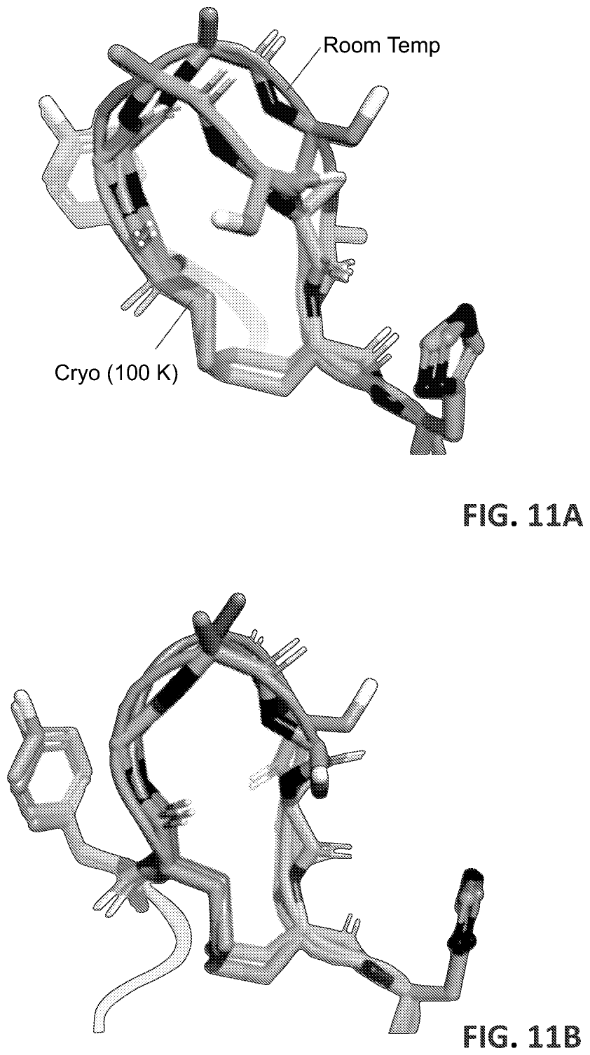

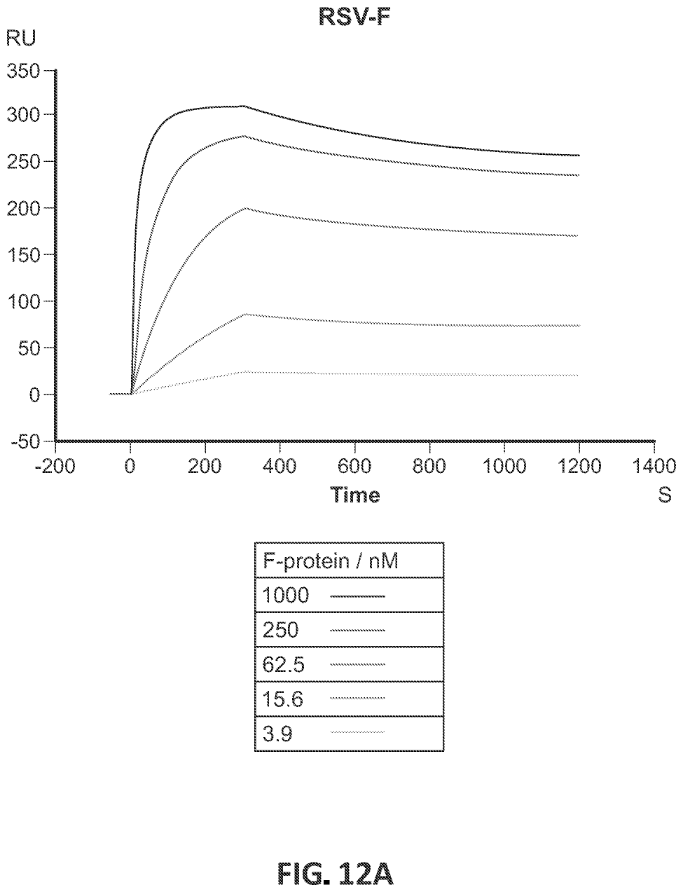

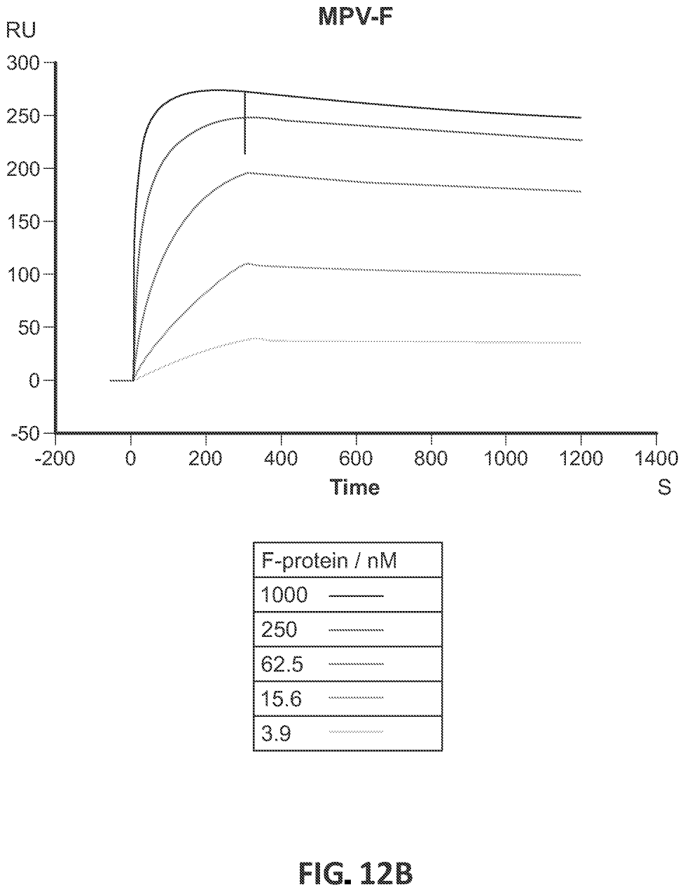

Rotated views of the determined and Fab structure are shown in FIGS. 10A-10D. MPH12 H-CDR3 occupies a shallow groove between L-CDR1 and L-CDR3. MPH12 H-CDR3 conformation at different temperatures was analyzed. At temperatures of 100 K and 293 K (room temperature), a disulfide bond was clearly observed (rotated views are shown in FIG. 11A and FIG. 11B). All other CDRs did not exhibit temperature-dependent conformation. Finally, MPH12 binding to purified RSV-F (FIG. 12A) or purified MPV-F D280 (FIG. 12B) was measured by SPR using various concentrations of antigen.

### Example 10

### Design and Testing of MPH12 Variant Antibodies

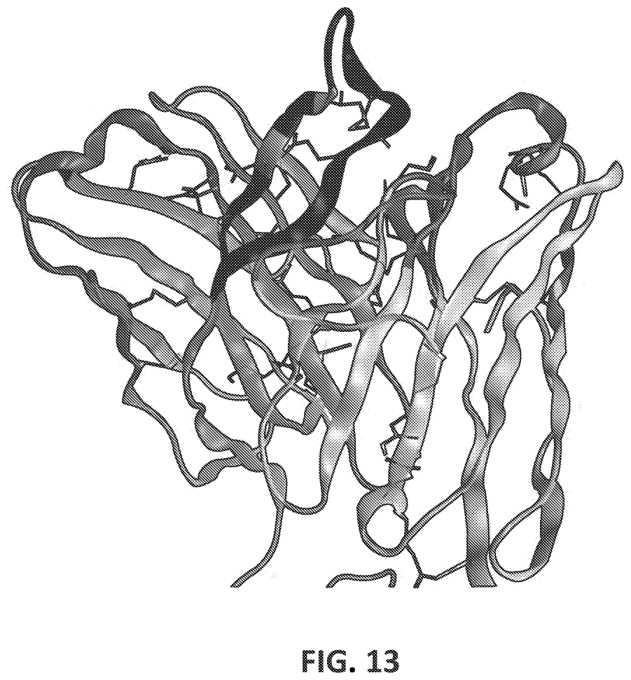

The amino acid sequences of the MPH12 heavy and light chains were analyzed using Molecular Operating Environment (MOE; Chemical Computing Group). No unpaired cysteines, N-linked glycosylation sites, or Asn deamidations in HCDR2 or LCDR1 were identified. As discussed above, the HCDR3 of MPH12 contains 2 SHM-encoded cysteines that have been experimentally shown to pair with one another. One potential methionine oxidation was found in the HCDR1, however, based on the Fab structure (FIG. 13) the methionine is completely buried. Two Asp isomerization sites were identified in HCDR2 (D62) and LCDR1 (D25) (using the IMGT numbering system). Substitution of D62 with Glutamic acid or substitution of S63 with Alanine were introduced to remove the DS motif in the HCDR2. Substitution of D25 with the germline residue serine or with the structurally similar glutamic acid or substitution of S26 with Alanine were considered to remove the DS motif in LCDR1. These mutations were introduced alone or in combination with mutations restoring germline residues in VH and VL framework regions, respectively. One additional variant was prepared wherein the cysteine residues of the HCDR3 were replaced by prolines. The combinations of VH and VL used to produce 49 variants of MPH12 are summarized in Table 20.

TABLE 20
MPH12 Variants
VL

| | | | | | | FRGL- | FRGL- | FRGL- | |
|---|---|---|---|---|---|---|---|---|---|
| | | FRGL | D25S | D25E | S26A | D25S | D25E | S26A | WT |
| VH | FRGL | V1 | V7 | V13 | V19 | V25 | V31 | V37 | V43 |
| | D62E | V2 | V8 | V14 | V20 | V26 | V32 | V38 | V44 |
| | S63A | V3 | V9 | V15 | V21 | V27 | V33 | V39 | V45 |
| | FRGL- | V4 | V10 | V16 | V22 | V28 | V34 | V40 | V46 |
| | D62E | | | | | | | | |
| | FRGL- | V5 | V11 | V17 | V23 | V29 | V35 | V41 | V47 |
| | S63A | | | | | | | | |
| | WT | V6 | V12 | V18 | V24 | V30 | V36 | V42 | V48 |
| | 2CP | — | | | | | | | V49 |

Production titers of antibodies comprising different combinations of MPH12 variant heavy and light chains were assessed following transfection of ExpiCHO cells. Absolute Titer (mAbPAC) are summarized in Table 21. The boxes with underlined values correspond to variants showing an increased titer of more than 1.2-fold over the parental control (highlighted in bold and italics). Several variants showed similar or increased titer compared with the parental antibody.

TABLE 21
MPH12 Variants Absolute Titers (mAbPAC)

| | | | | | MPH12 | MPH12 | MPH12 | |
|---|---|---|---|---|---|---|---|---|
| | MPH12 | MPH12 | MPH12 | MPH12 | VL- | VL- | VL- | |
| | VL- | VL- | VL- | VL- | FRGL- | FRGL- | FRGL- | MPH12 |
| | FRGL | D25S | D25E | S26A | D25E | S26A | S26A | VL |
| MPH12 | 422.3 | 293.3 | 224.2 | 226.8 | 276.0 | 226.8 | 209.4 | 236.9 |
| VH- | | | | | | | | |
| FRGL | | | | | | | | |
| MPH12 | 353.1 | 293.3 | 297.1 | 278.4 | 268.4 | 252.9 | 240.9 | 239.9 |
| VH-D62E | | | | | | | | |
| MPH12 | 424.1 | 364.1 | 241.3 | 261.0 | 259.6 | 209.4 | 187.7 | 193.7 |
| VH-S63A | | | | | | | | |
| MPH12 | 495.0 | 303.1 | 258.0 | 265.4 | 299.9 | 256.4 | ND | 267.8 |
| VH- | | | | | | | | |
| FRGL- | | | | | | | | |
| S63A | | | | | | | | |
| MPH12 | 515.9 | 279.8 | 257.2 | 283.2 | 301.5 | 234.9 | 279.6 | 226.2 |
| VH- | | | | | | | | |
| FRGL- | | | | | | | | |
| D62E | | | | | | | | |
| MPH12 | 334.8 | 359.1 | 199.3 | 226.4 | 219.4 | 255.5 | 280.8 | 225.6 |
| VH | | | | | | | | |
| MPH12 | ND | ND | ND | ND | ND | ND | ND | 308.6 |
| VH_2CP | | | | | | | | |

MPH12 variant antibodies were also assessed for binding to DS-Cav1 RSV F by ELISA. Table 22 presents the binding EC50 as assessed using 1 to 3 dilutions of DC-VCav1. Variants retaining parental antibody affinity or showing increased affinity are underlined.

TABLE 22
MPH12 Variants RSV Binding EC50 (ng/ml)

| | | | | | MPH12 | MPH12 | MPH12 | |
|---|---|---|---|---|---|---|---|---|
| | MPH12 | MPH12 | MPH12 | MPH12 | VL- | VL- | VL- | |
| | VL- | VL- | VL- | VL- | FRGL- | FRGL- | FRGL- | MPH12 |
| | FRGL | D25S | D25E | S26A | D25E | S26A | S26A | VL |
| MPH12 | 30.5 | 21.9 | 34.0 | 39.7 | 47.2 | 61.9 | 53.1 | 32.8 |
| VH- | | | | | | | | |
| FRGL | | | | | | | | |
| MPH12 | 26.7 | 23.6 | 27.4 | 31.0 | 37.4 | 46.9 | 48.7 | 24.1 |
| VH- | | | | | | | | |
| D62E | | | | | | | | |
| MPH12 | 25.4 | 24.9 | 40.6 | 25.8 | 42.0 | 41.7 | 71.8 | 35.8 |
| VH- | | | | | | | | |
| S63A | | | | | | | | |
| MPH12 | 15.3 | 15.6 | 36.8 | 32.2 | 34.7 | 48.9 | ND | 23.8 |
| VH- | | | | | | | | |
| FRGL- | | | | | | | | |
| S63A | | | | | | | | |
| MPH12 | 34.9 | 25.1 | 39.7 | 42.5 | 37.0 | 42.8 | 47.3 | 51.0 |
| VH- | | | | | | | | |
| FRGL- | | | | | | | | |
| D62E | | | | | | | | |
| MPH12 | 35.8 | 23.5 | 43.8 | 50.8 | 30.6 | 33.5 | 38.5 | 32.6 |
| VH | | | | | | | | |
| MPH12 | ND | ND | ND | ND | ND | ND | ND | NC |
| VH_2P | | | | | | | | |

Based on titer and binding affinity results, six MPH12 variant antibodies were selected for further characterization: MPH12-v16 (VH-FRGL-S63A/VL-D25S), MPH12-v17 (VH-FRGL-D62E/VL-D25S), MPH12-v28 (VH-FRGL-S63A/VL-S26A), MPH12-v29 (VH-FRGL-D62E/VL-S26A), MPH12-v34 (VH-FRGL-S63A/VL-FRGL-D25S), and MPH12-v35 (VH-FRGL-D62E/VL-FRGL-D25S). These variants have sequences as set forth in Tables 2 and 20, with reference to Table 1 and the Sequence Listing.

### Example 11

### Further Characterization of Selected MPH12 Variant Antibodies

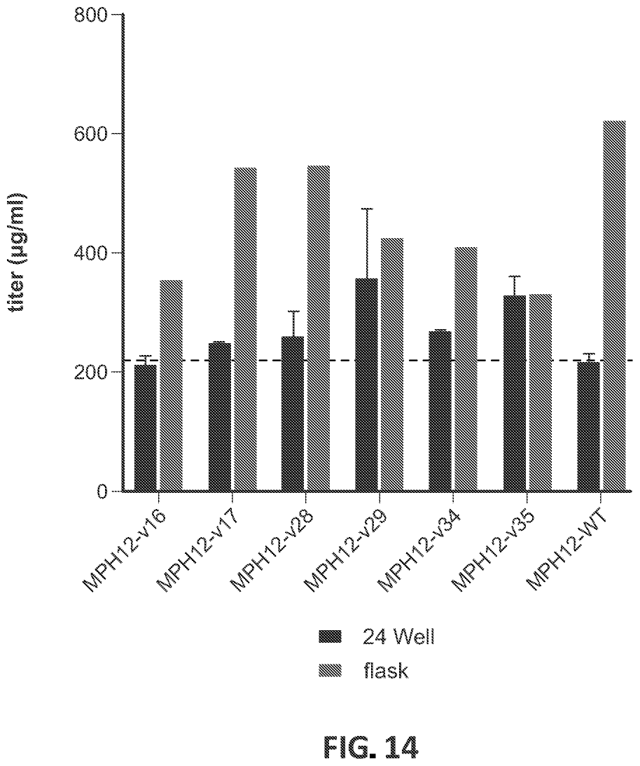

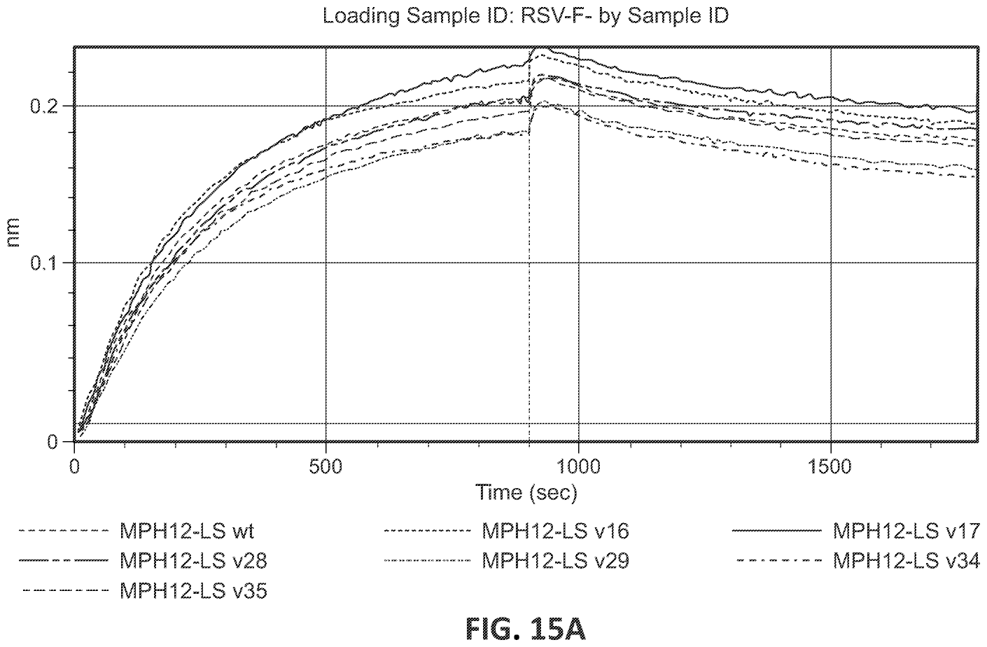

The six selected MPH12 variant antibodies from Example 10 were generated via transfection in ExpiCHO cells according to the manufacturer's instruction in a 100 ml flask format. MPH12 (parental MPH12-wt) was included a comparator. Antibodies also contained a M428L/N434S (LS) mutation. Titers were determined using monoclonal antibody protein A columns (mAb-PAC™) from supernatants collected at day 8 post-transfection (FIG. 14). Most variants showed similar titer compared with the parental antibody, though some of the variants showed an increased titer. The 24-well expression was performed once.

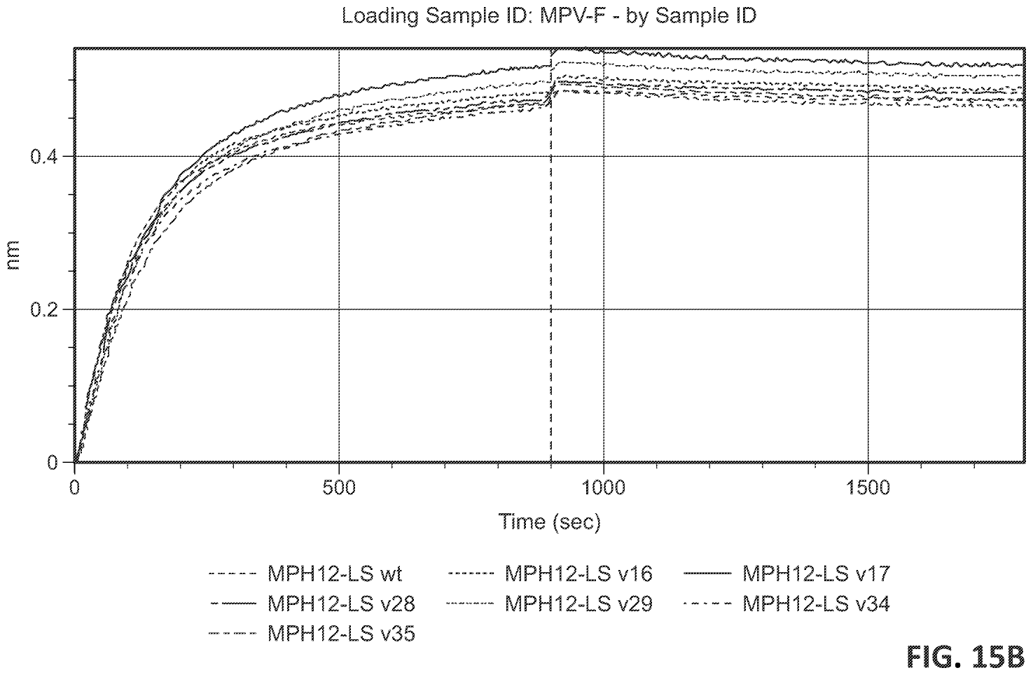

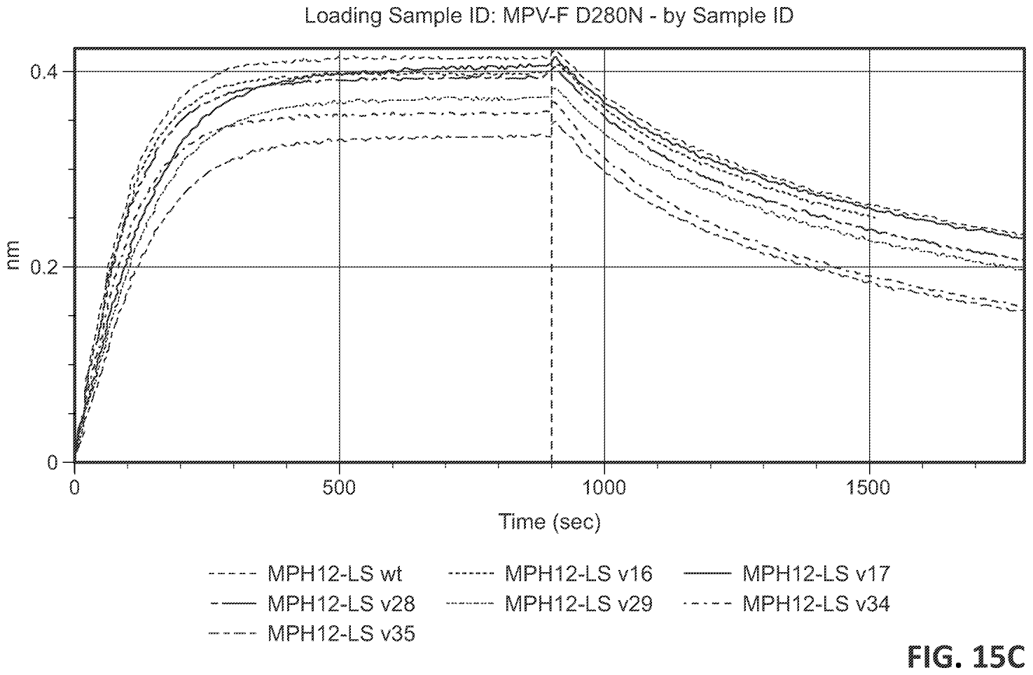

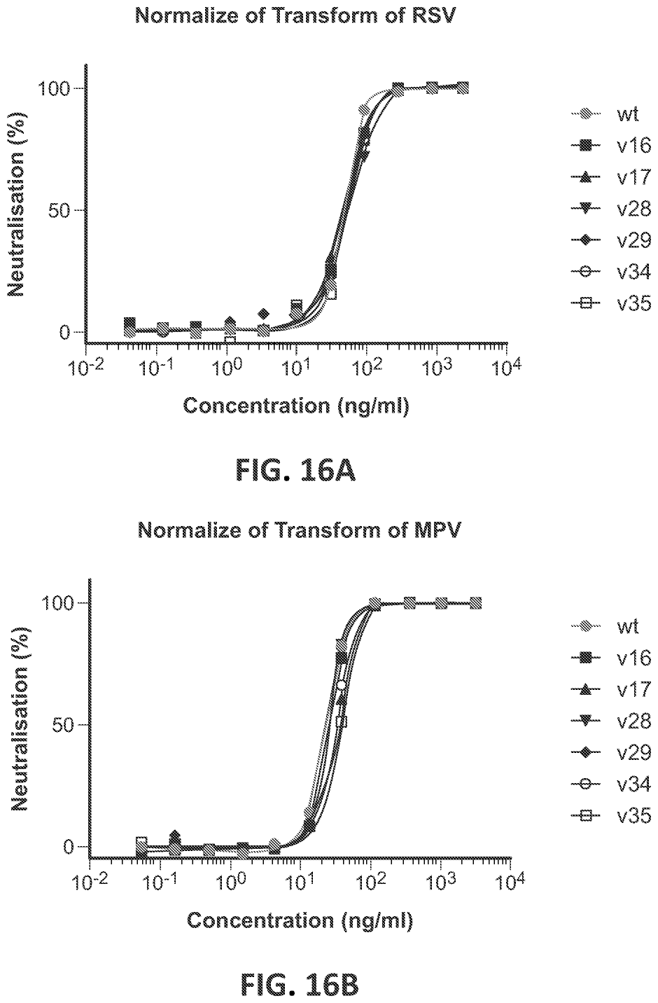

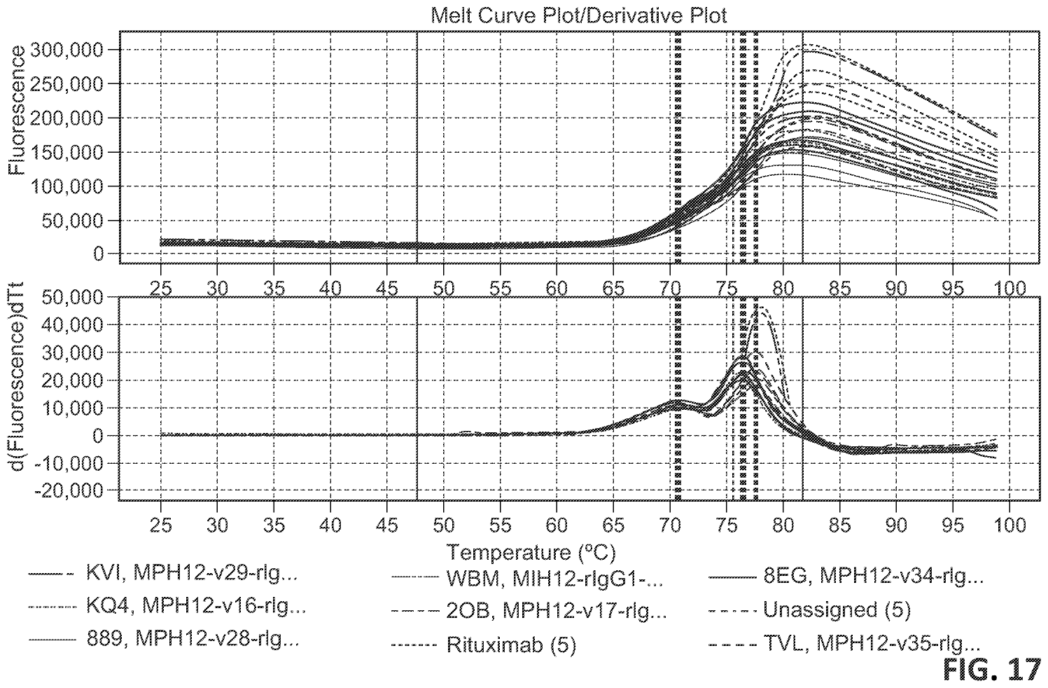

Binding to RSV-F (FIG. 15A), MPV-F D280 protein (FIG. 15B), and MPV-F N280 protein (FIG. 15C) was assessed by biolayer interferometry (BLI). All antibodies were expressed as rIgG1 containing MLNS mutations in the Fc. All variants retained similar binding affinity as the parental mAb. Neutralization of RSV (FIG. 16A) and MPV (FIG. 16B) was also tested using a GFP-based in vitro neutralization assay (Table 23). All six MPH12 variant antibodies showed comparable neutralizing potency to one another and to the parental mAb.

TABLE 23
MPH12 Variants Neutralization of RSV and MPV (non-D280N)

| | RSV IC 50 (ng/mL) | MPV IC50 (ng/mL) |
|---|---|---|
| WT | 47.7 | 24.55 |
| v16 | 50.34 | 26.82 |
| v17 | 44.93 | 30.44 |
| v28 | 59.40 | 26.33 |
| v29 | 60.27 | 35.25 |
| v34 | 48.65 | 31.87 |
| v35 | 57.54 | 39.92 |

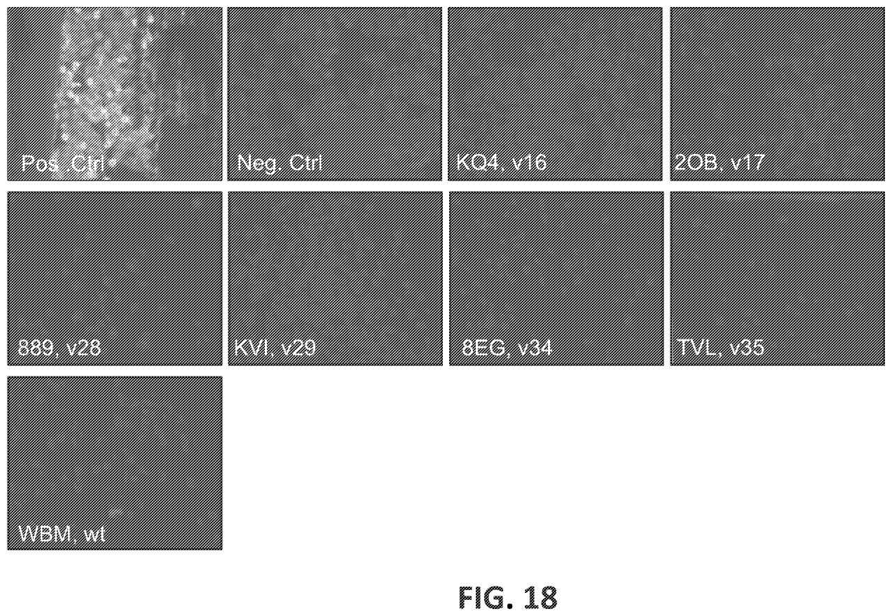

Thermal stability of the six MPH12 variant antibodies was assessed using a ProteinShift assay (FIG. 17), wherein a S26A mutation in the VL in combination with a S63A mutation in the VH (MPH12-v28) appeared to produce a destabilizing effect. Finally, MPH12 and the MPH12 variant antibodies did not exhibit polyreactivity using Eurimmune 293 slides (FIG. 18).

Titer, thermal stability, binding, formation of aggregates, and neutralization data results for the six MPH12 variant antibodies from these studies are summarized in Table 24 and Table 25. Characteristics for MPH12 parental antibody (“WT”) are shown at the bottom of the table.

TABLE 24
MPH12 Variants Results-1

| aB | Titer Flask | Titer 24 well | P yield | Tm1 | Tm2 | Tm (DSF) |
|---|---|---|---|---|---|---|
| 16 | 355.1 | 213.7 | 117.13 | 70.75 | 76.42 | 68.86 |
| 17 | 544.5 | 248.95 | 82.77 | 70.62 | 77.64 | 69.70 |
| 28 | 549.2 | 260.9 | 84.94 | NC | 75.7 | 67.54 |
| 29 | 425.1 | 360.2 | 85.33 | 70.54 | 76.87 | 68.69 |
| 34 | 411.5 | 271.3 | 95.92 | 70.92 | 76.45 | 68.88 |
| 35 | 334.4 | 329.1 | 91.08 | 70.53 | 77.61 | 70.13 |
| WT | 623.5 | 218.1 | 103.88 | 71.03 | 76.9 | 68.9 |

TABLE 25
MPH12 Variants Results-2

| | RSV-F | MPV-F D280 | MPV-F N280 | RSV | MPV |
|---|---|---|---|---|---|
| aB | KD (M) | KD (M) | KD (M) | Neut | Neut |
| 16 | 4.1E−10 | 3.8E−11 | 5.4E−10 | 50.34 | 26.82 |
| 17 | 3.9E−10 | 2.8E−11 | 5.5E−10 | 44.93 | 30.44 |
| 28 | 3.8E−10 | 4.5E−11 | 7.4E−10 | 59.4 | 26.33 |
| 29 | 3.4E−10 | 2.7E−11 | 6.4E−10 | 60.27 | 35.25 |
| 34 | 5.0E−10 | 3.9E−11 | 8.2E−10 | 48.65 | 31.87 |
| 35 | 4.6E−10 | 2.8E−11 | 7.8E−10 | 57.54 | 39.92 |
| WT | 4.2E−10 | 2.6E−11 | 1.1E−09 | 47.7 | 24.55 |

MPH12-v16 and MPH12-v34 were selected for further functional characterization.

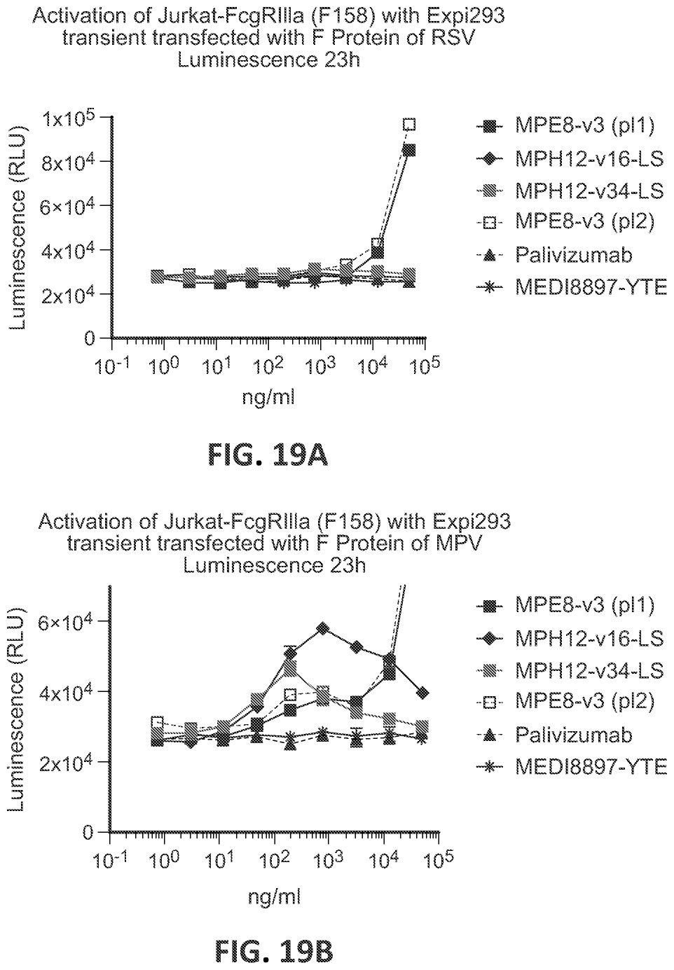

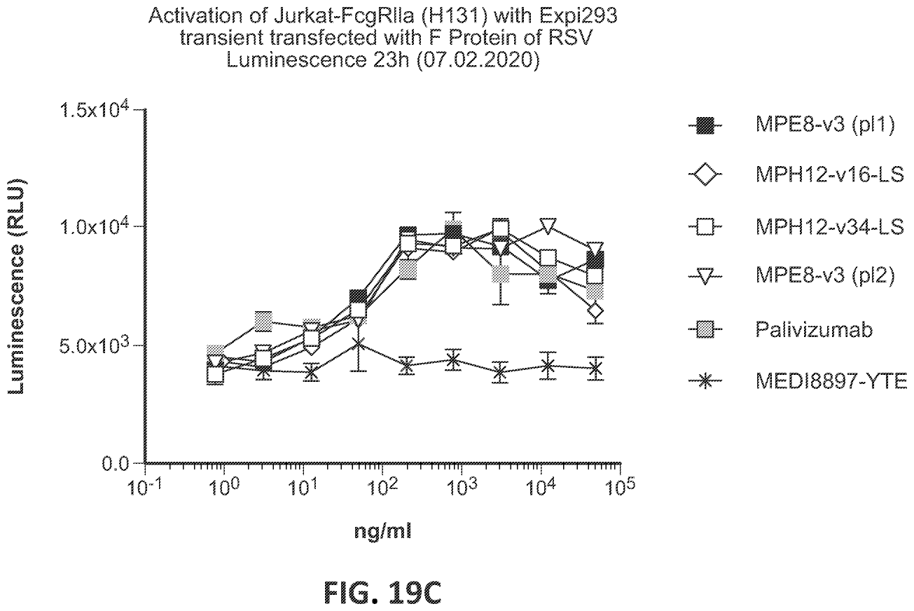

MPH12-v16 and MPH12-v34 were evaluated for activation of FcγRIIIa and FcγRIIa using a NFAT-driven luciferase reporter assay. Activation of Jurkat-FcγRIIIa (F158 allele) cell lines was assessed following incubation with Expi293 cells transiently transfected with fusion glycoprotein of RSV (FIG. 19A) or fusion glycoprotein of MPV (FIG. 19B). Activation of Jurkat-FcγRIIa (H131 allele) cell lines was assessed following incubation with Expi293 cells transiently transfected with fusion glycoprotein of RSV (FIG. 19C). Comparator antibodies, MPE8-v3 (p11), MPE8-v3 (p12), palivizumab, and MED18897-YTE, were also tested. In this and other Examples, palivizumab, unless otherwise indicated, may refer to an antibody containing the VH and VL of commercially available palivizumab, including antibodies containing a different Fc portion than commercially available palivizumab, as such antibodies are expected to retain antigen-binding characteristics of palivizumab.

MPH12-v16 is tested in additional studies, including an in vivo study using a cotton rat model of infection.

### Example 12

### Competition/Binning of MPK Antibodies

### Experiment 1

Competition/binning assays were conducted as follows:

Materials— Kinetics Buffer 1× no tween: 0.01% BSA in PBS; SAX sensors.

Conditions— SAX sensor; 384 well plate; volume: 70 ul/well; pH 7.1, 1000 rpm, 30° C.; Sensor Hydratation: >10 min in KB; Loading Antigen: 0.5 μg/ml, 8 min in KB; Association of mAbs: 15 μg/ml in KB, 7 min; Baselines: in KB, 30 sec.

Procedure—Pre-hydrate sensors in KB (see conditions) for at least 10 min; In the meanwhile prepare plate for loading; Perform RUN: Baseline in A1→Loading in A2→Baseline in A1→Association 1 in A3→Association 2 in A4.

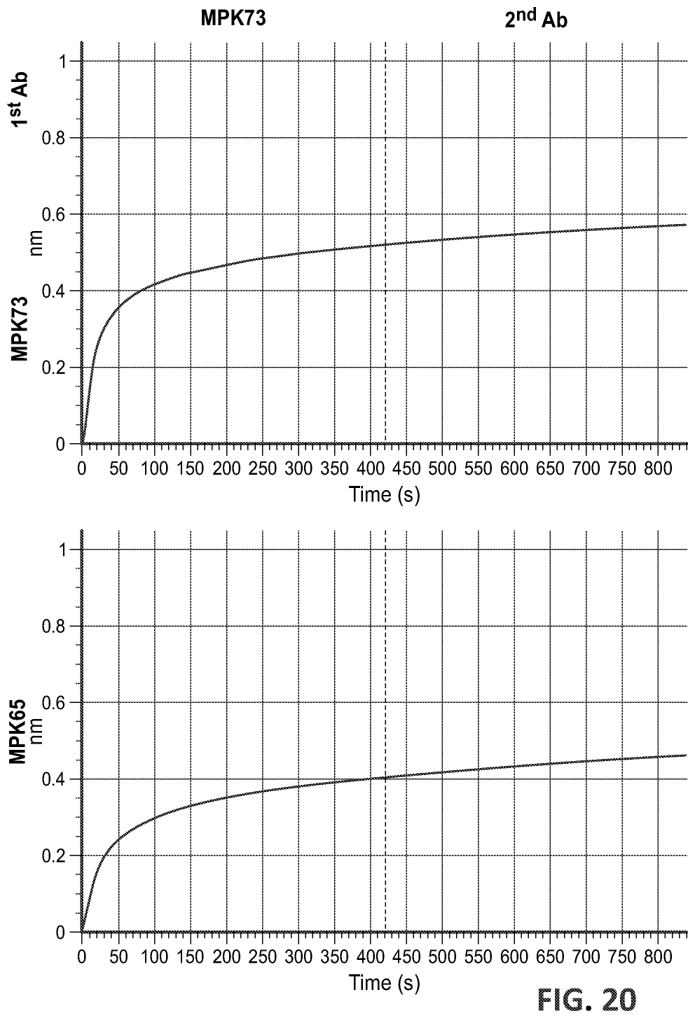

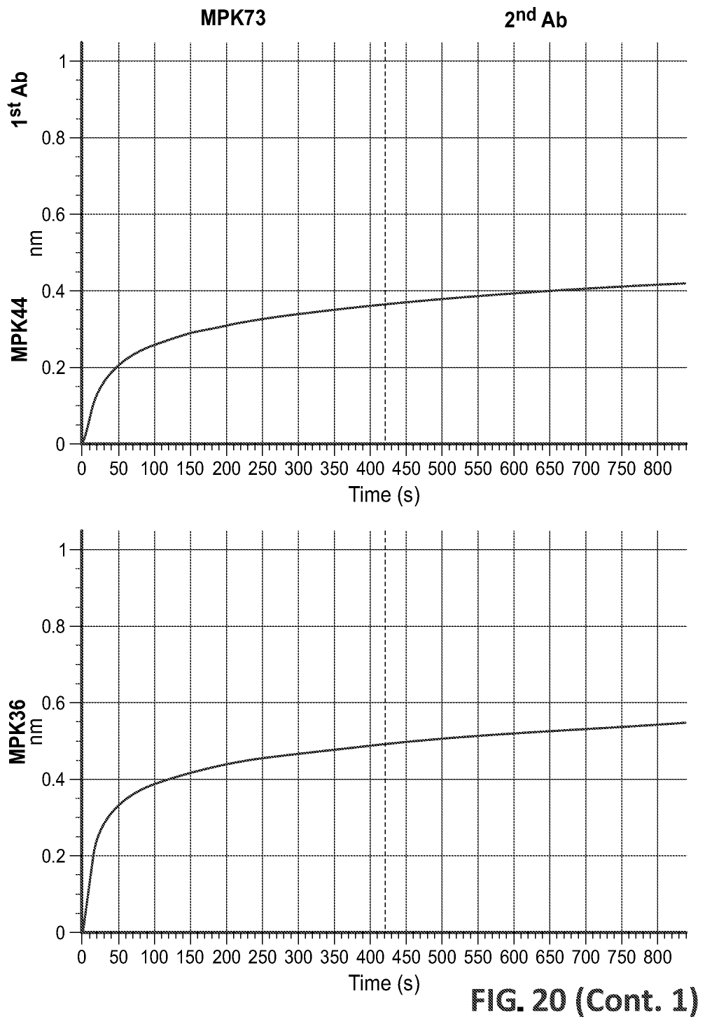

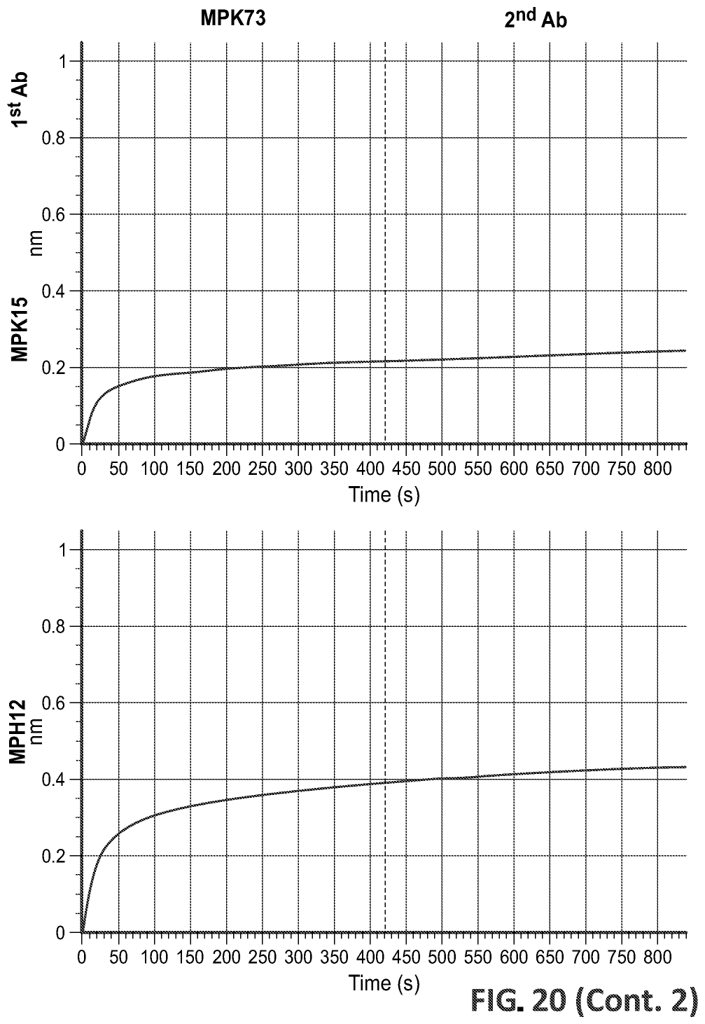

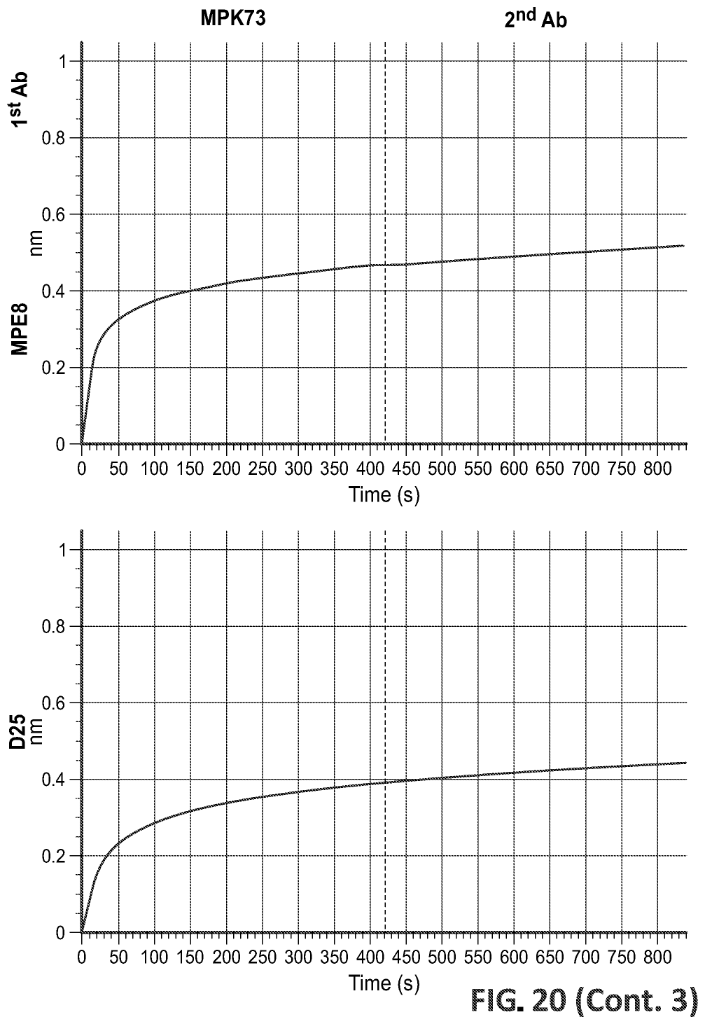

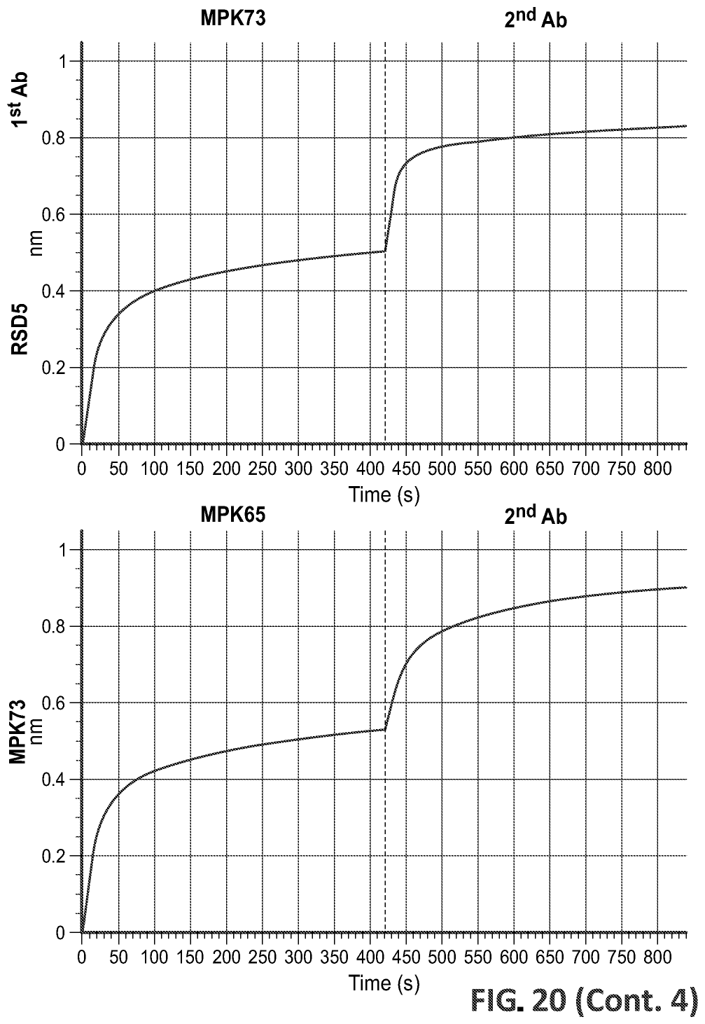

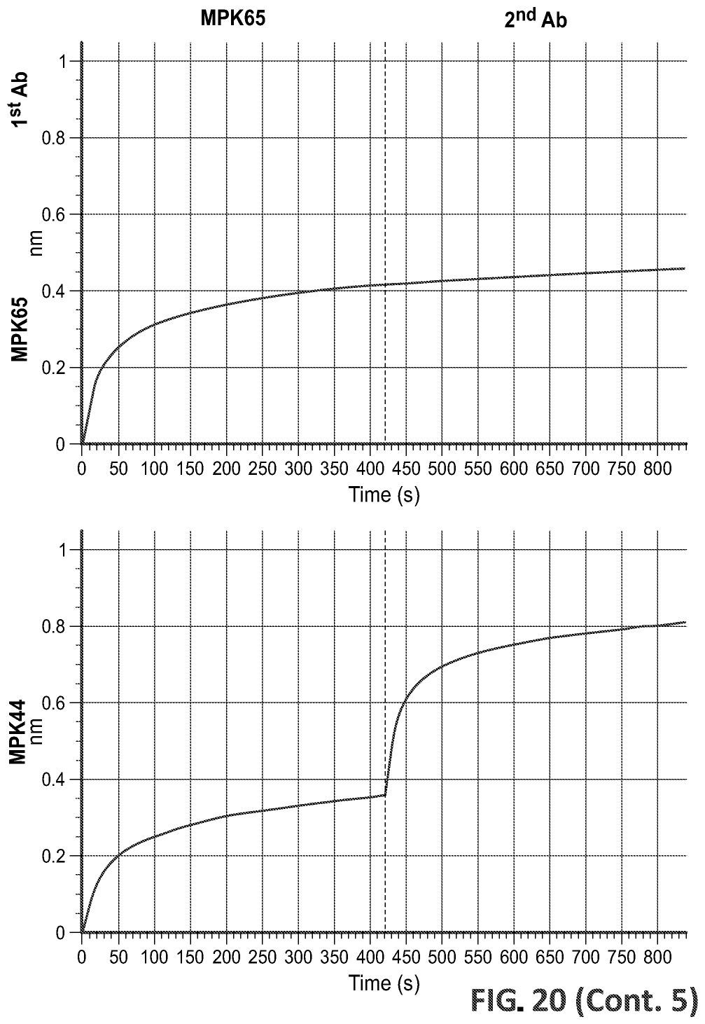

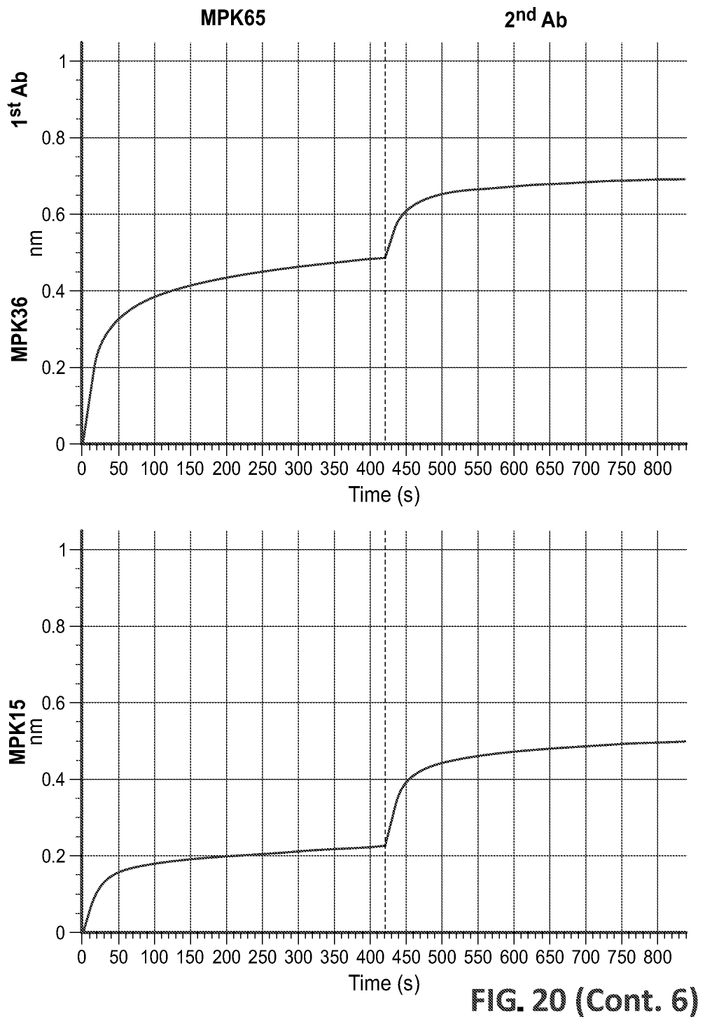

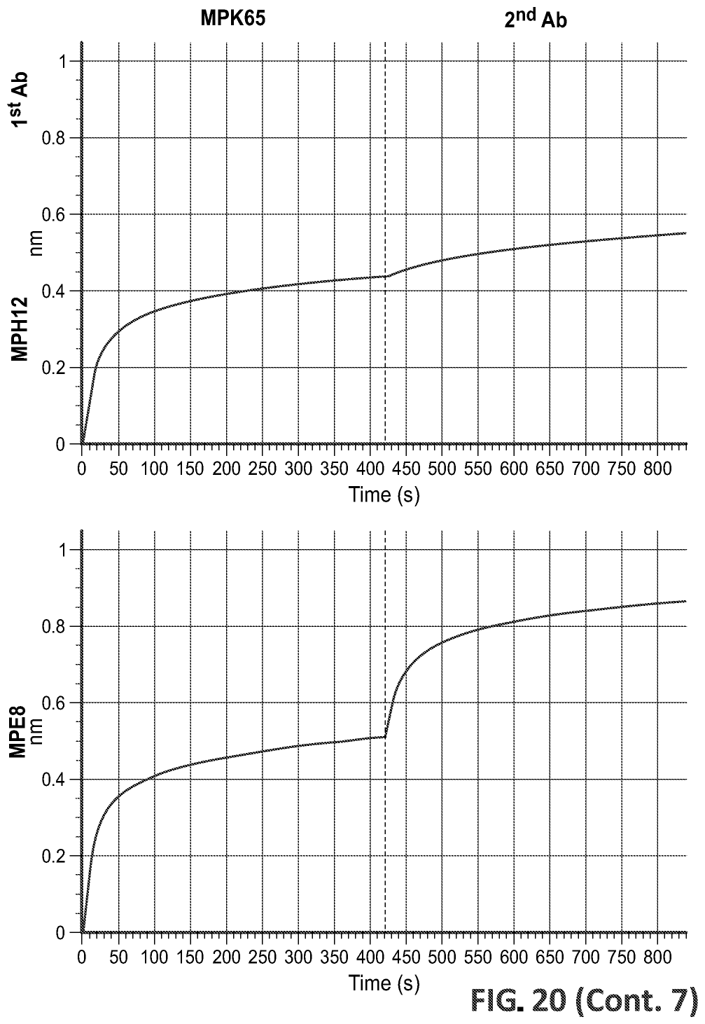

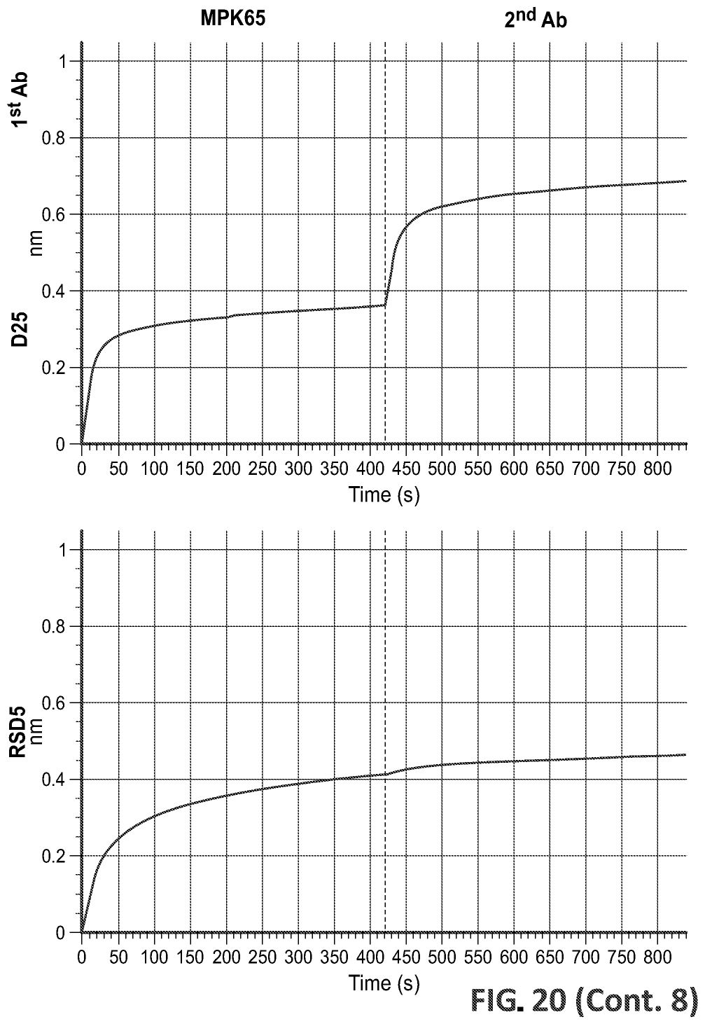

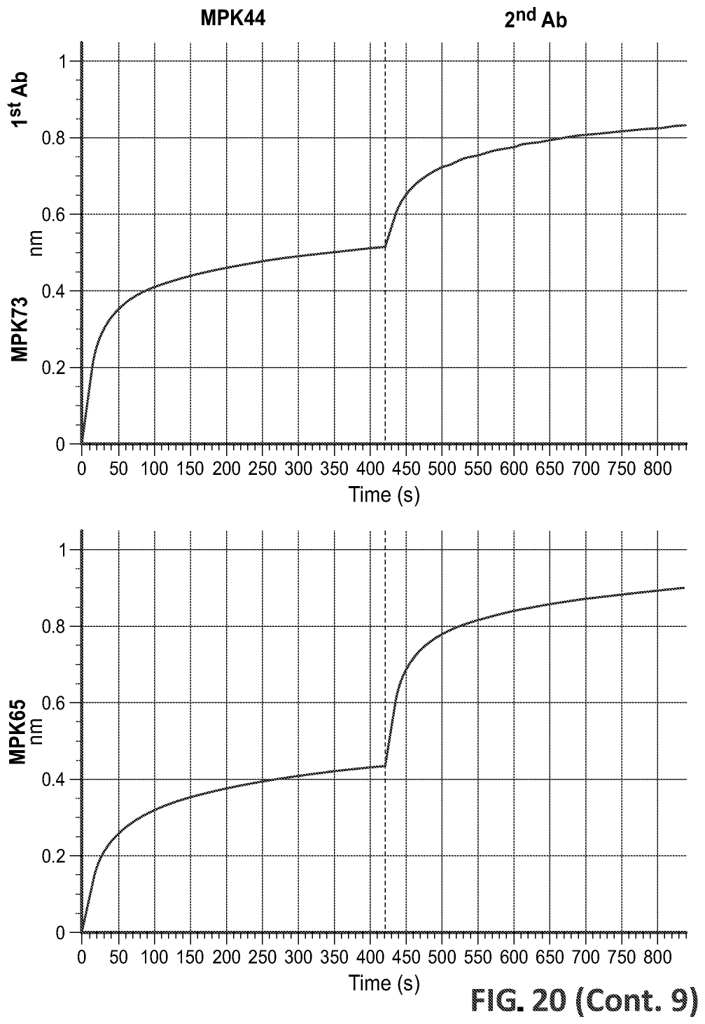

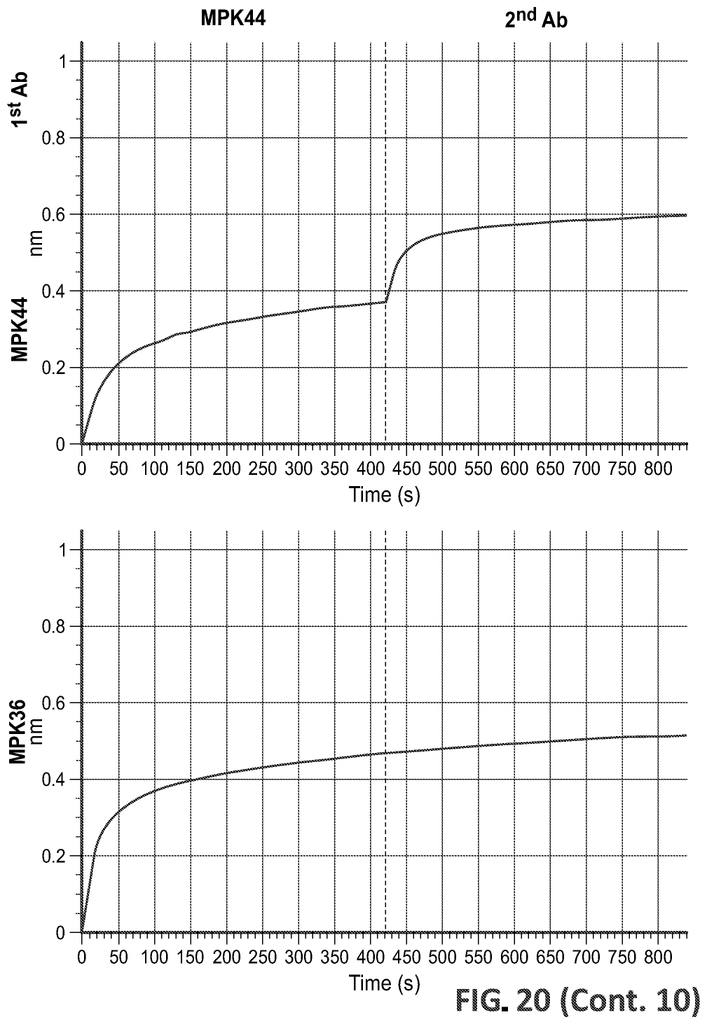

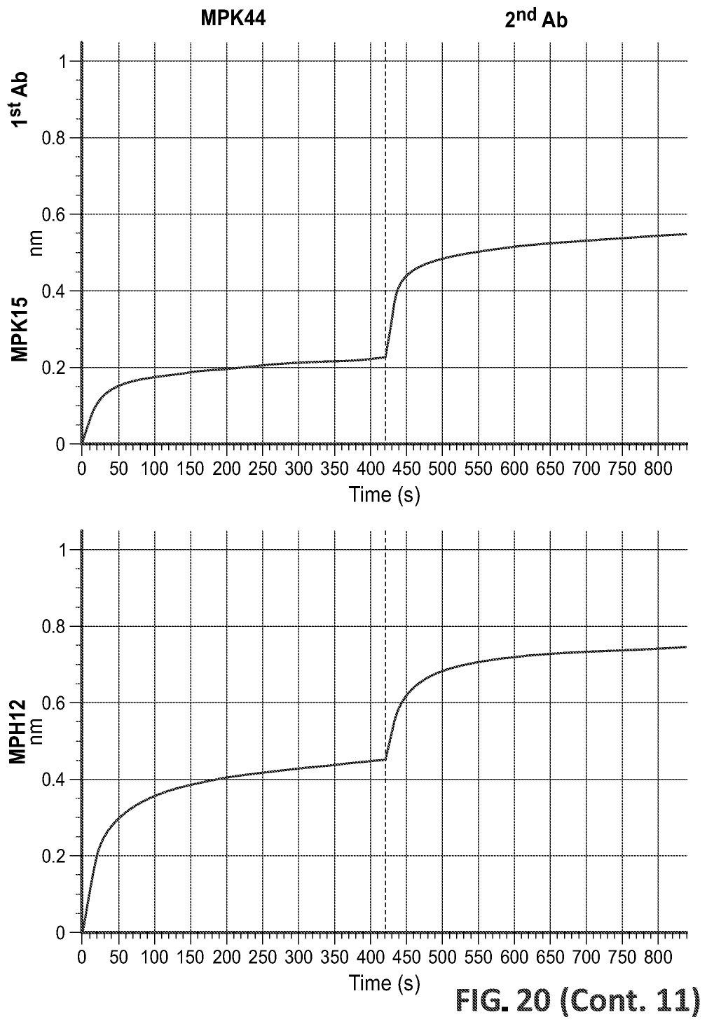

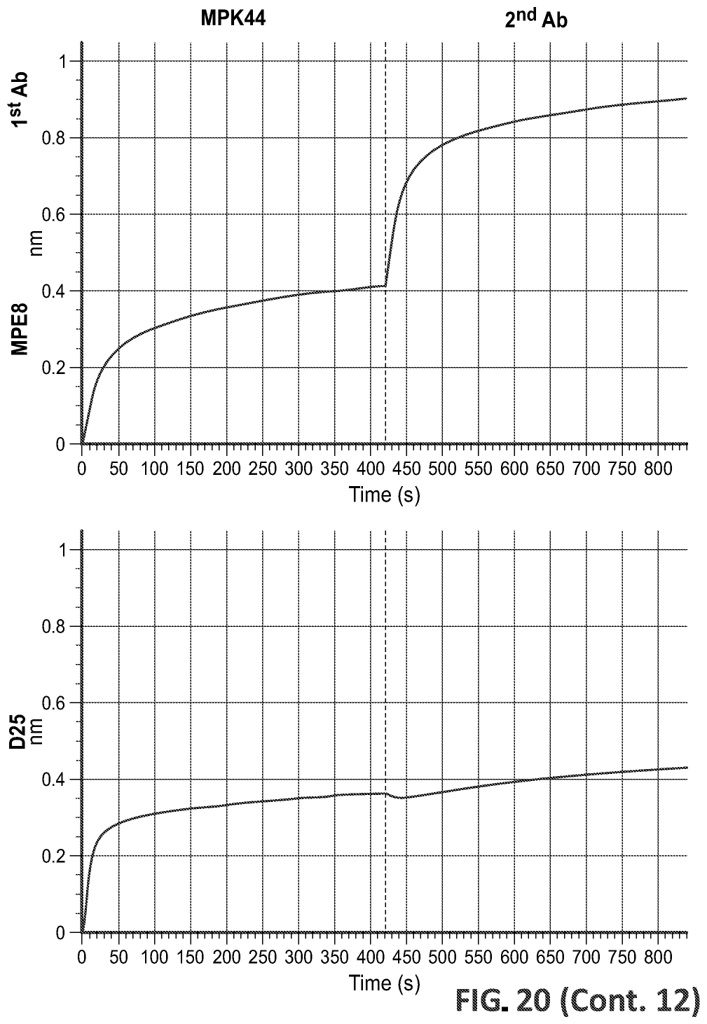

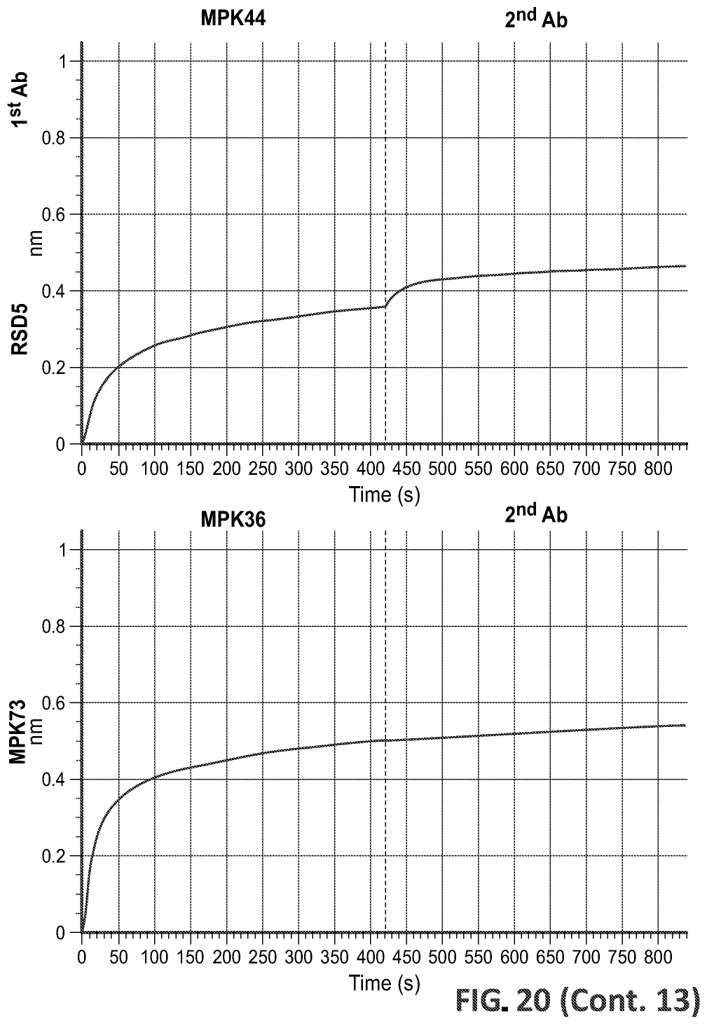

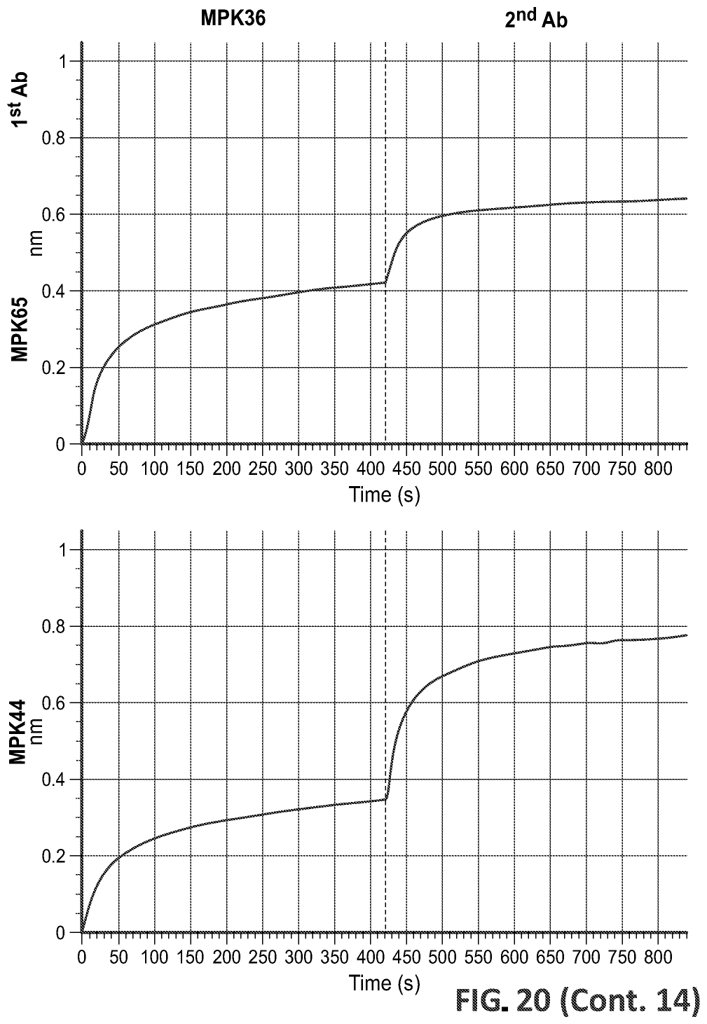

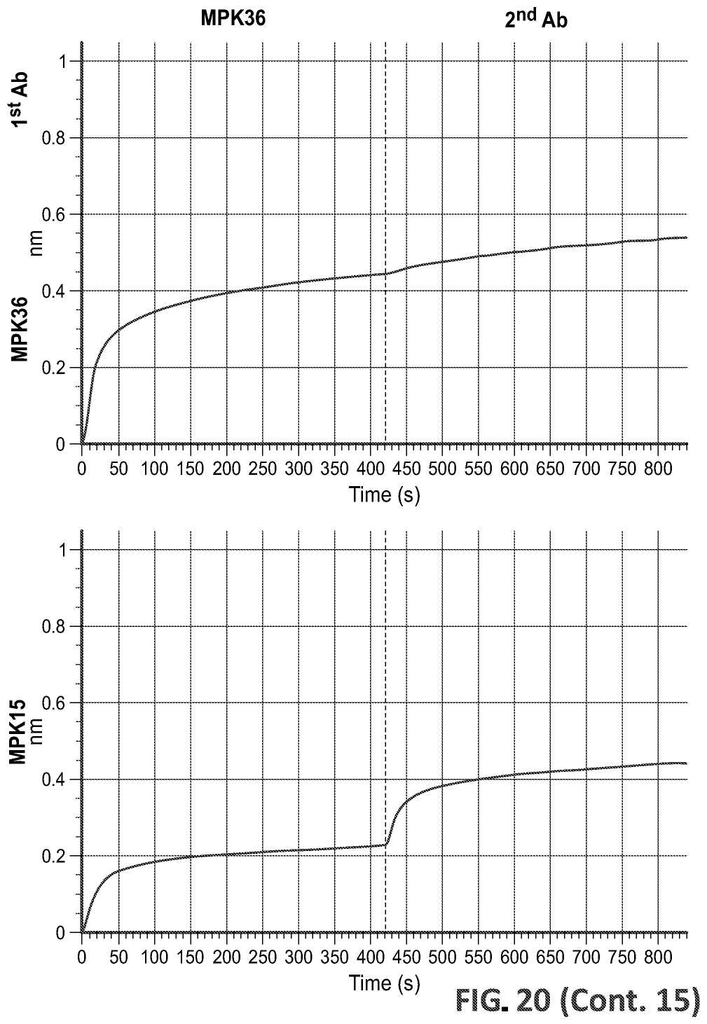

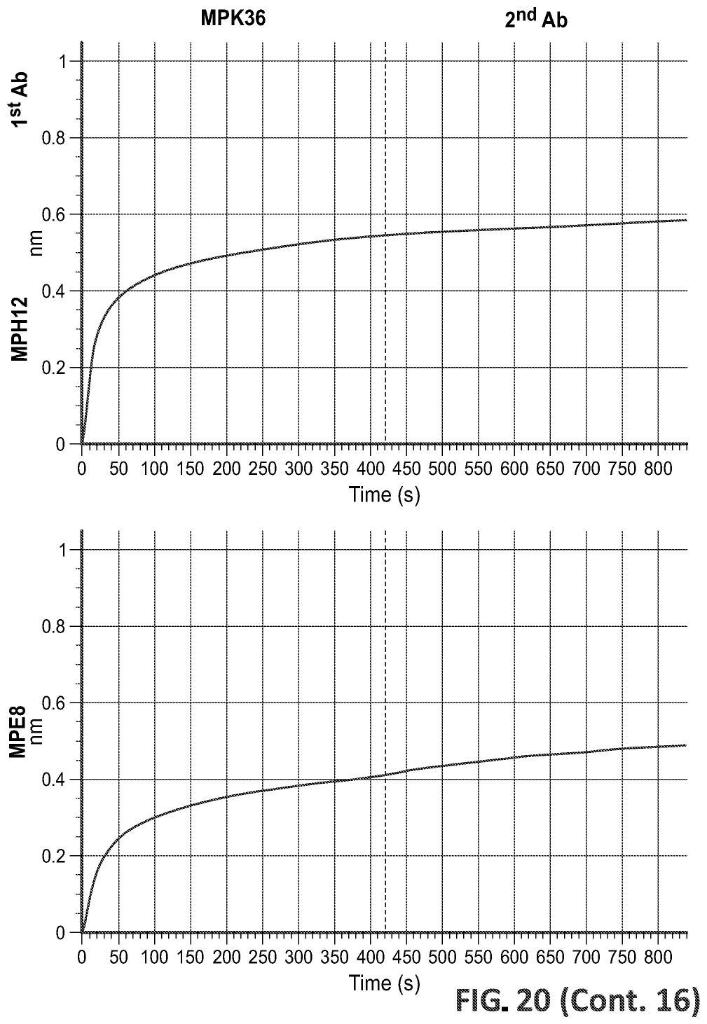

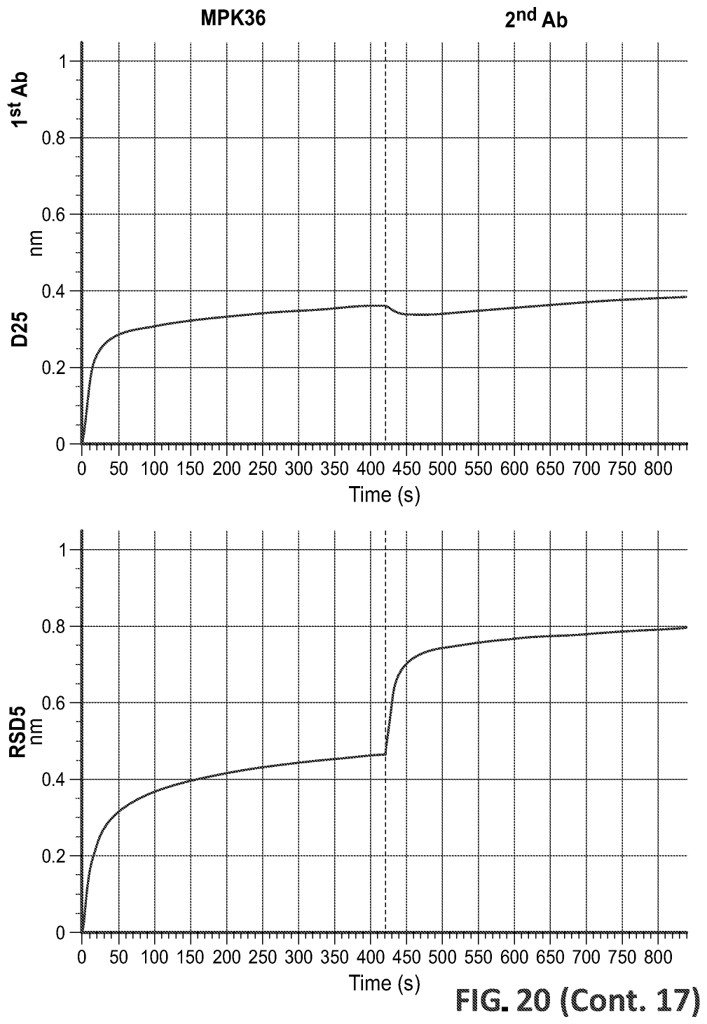

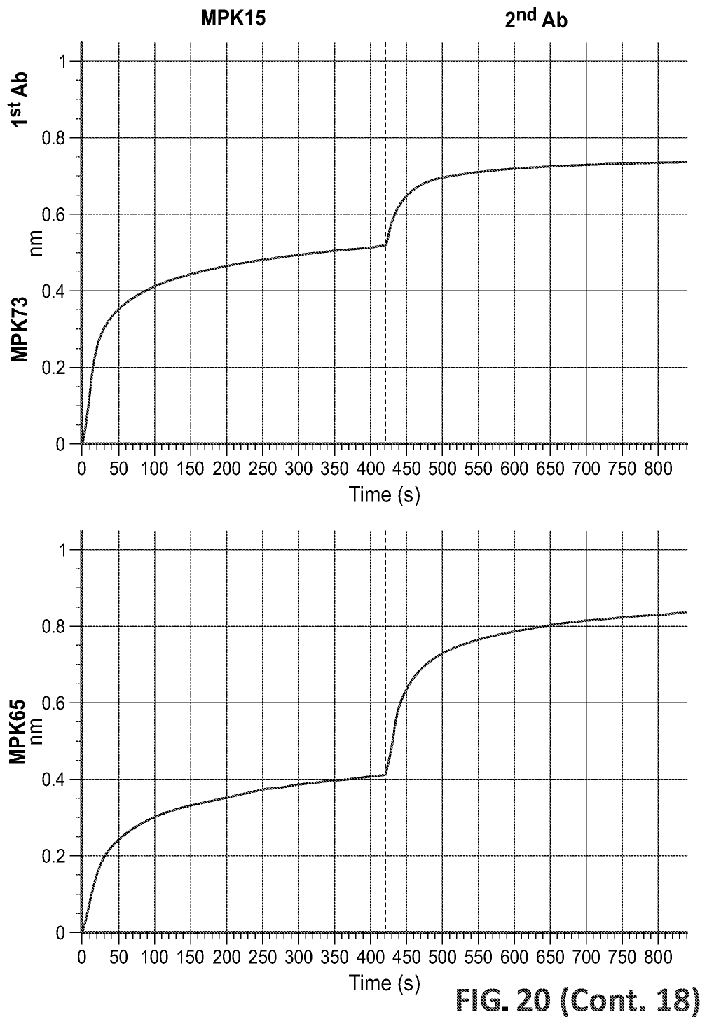

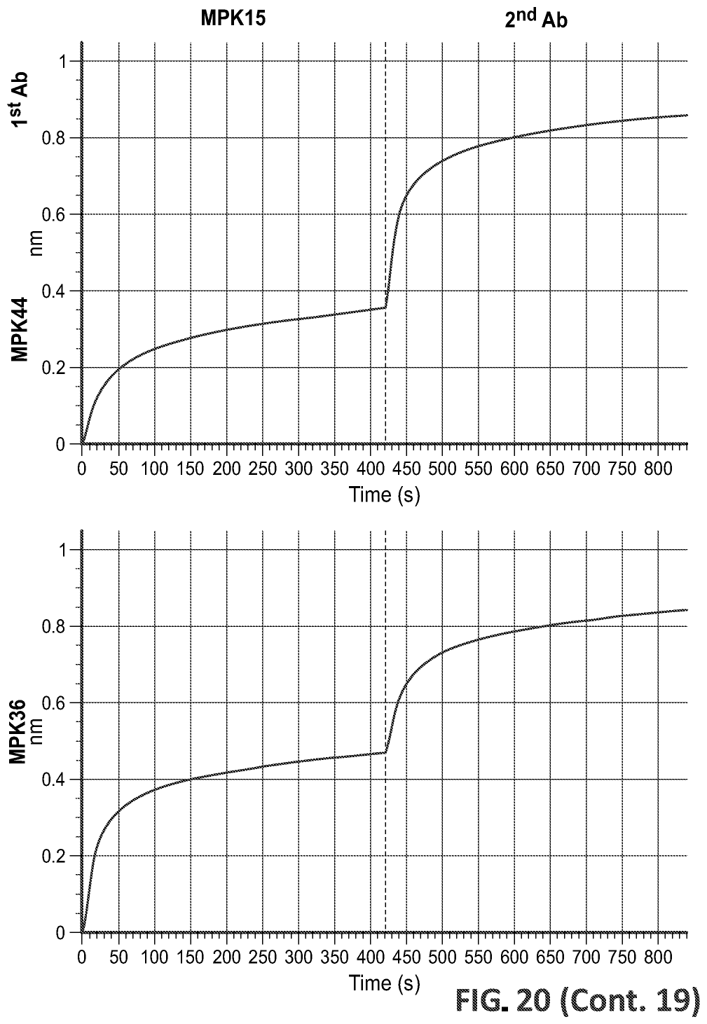

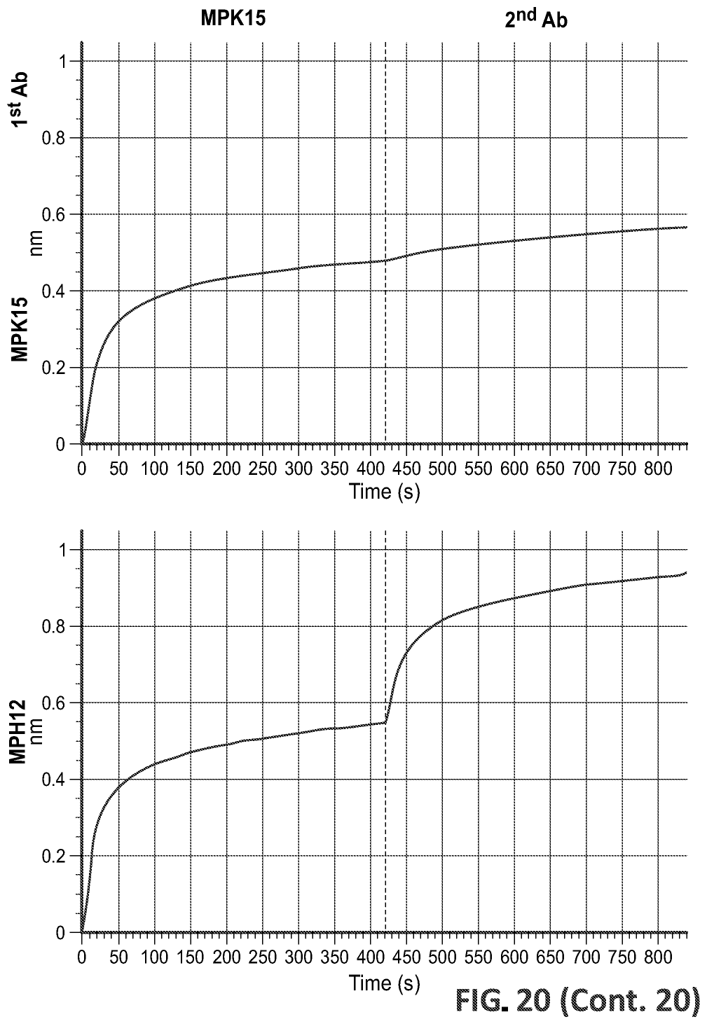

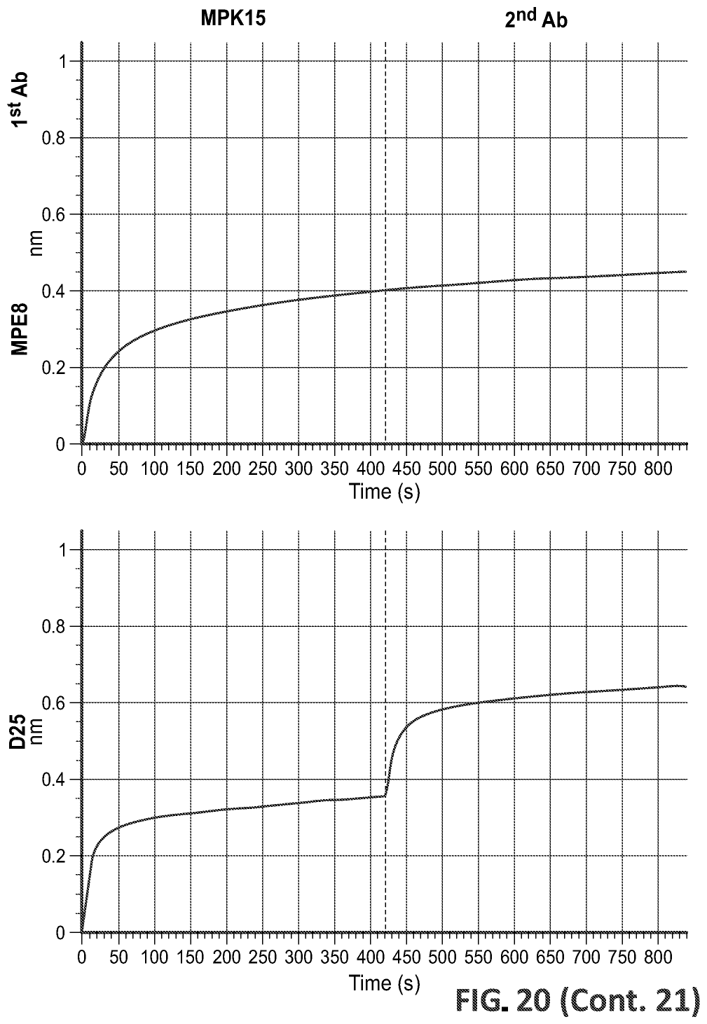

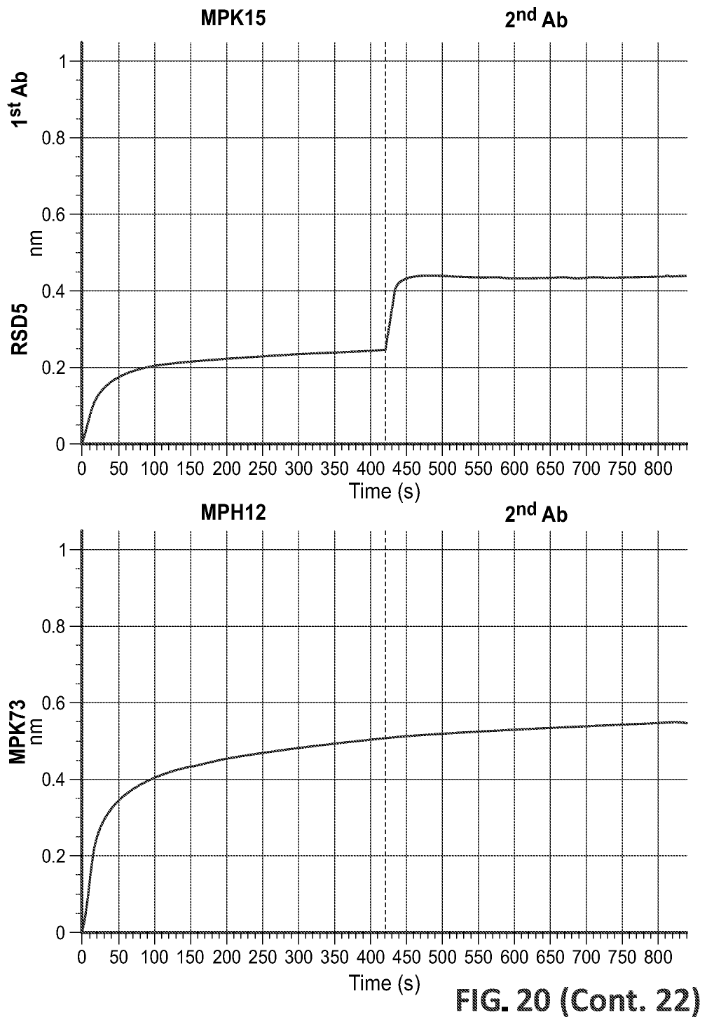

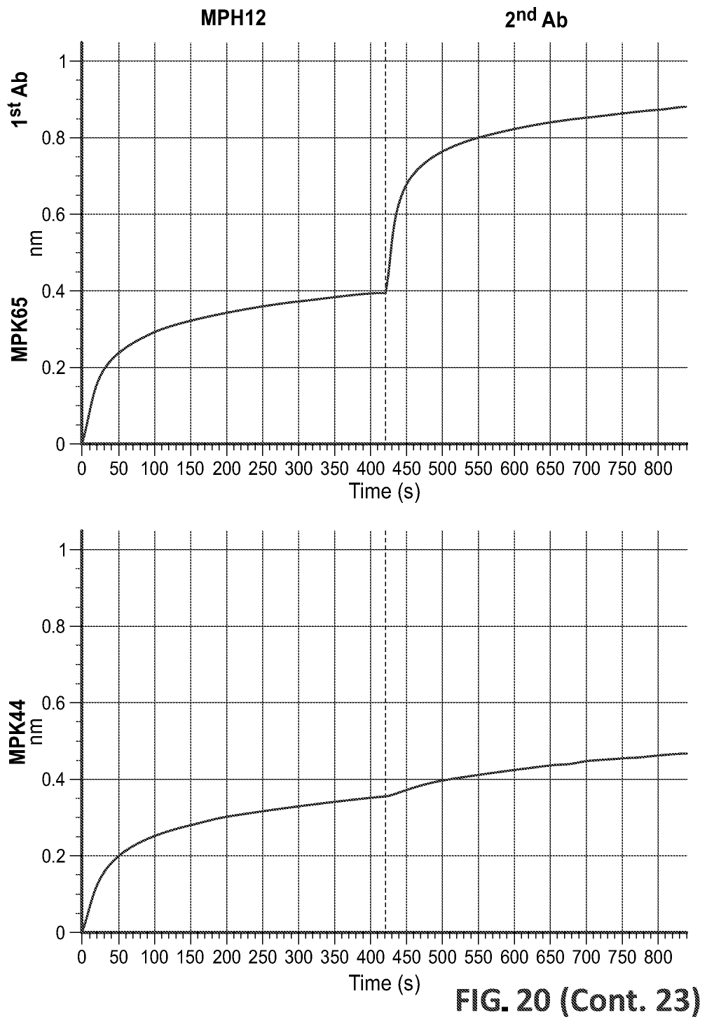

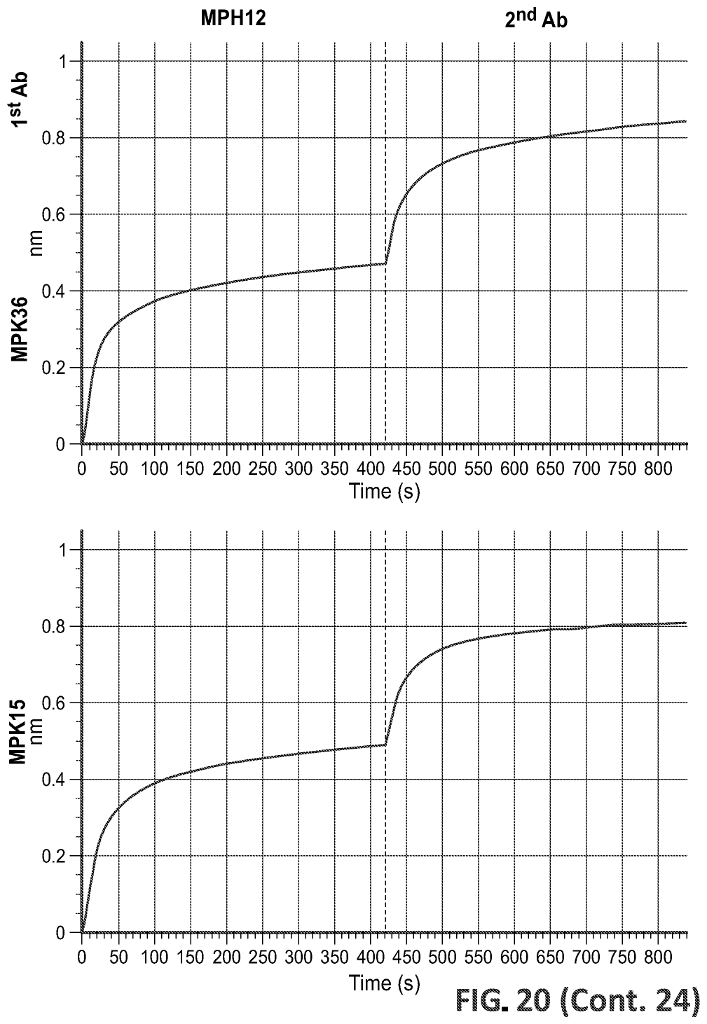

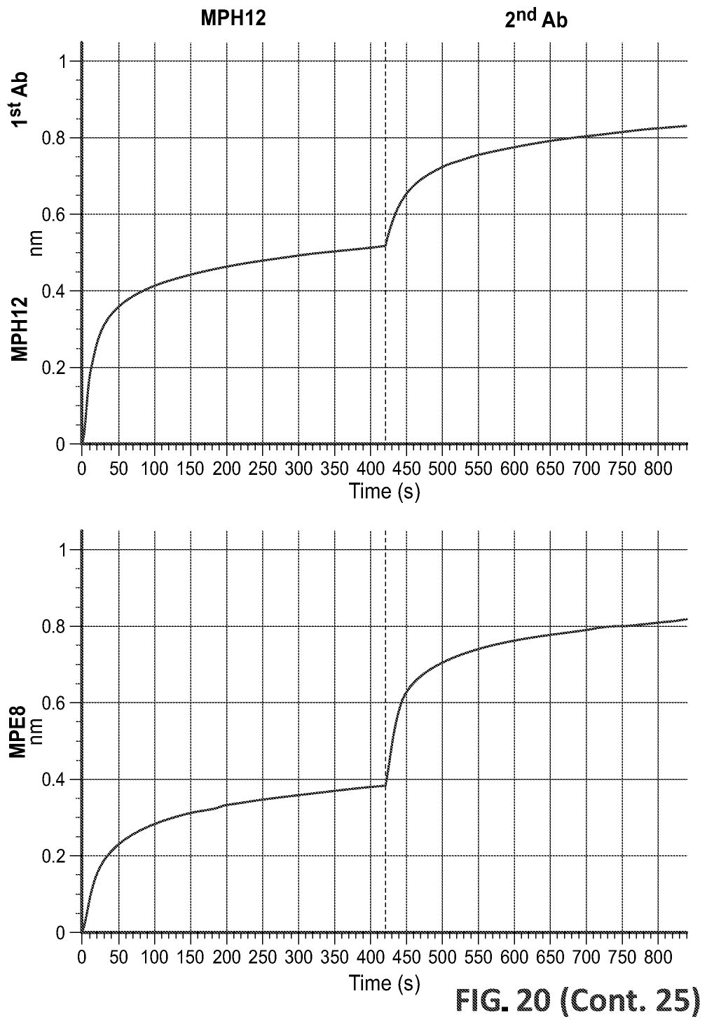

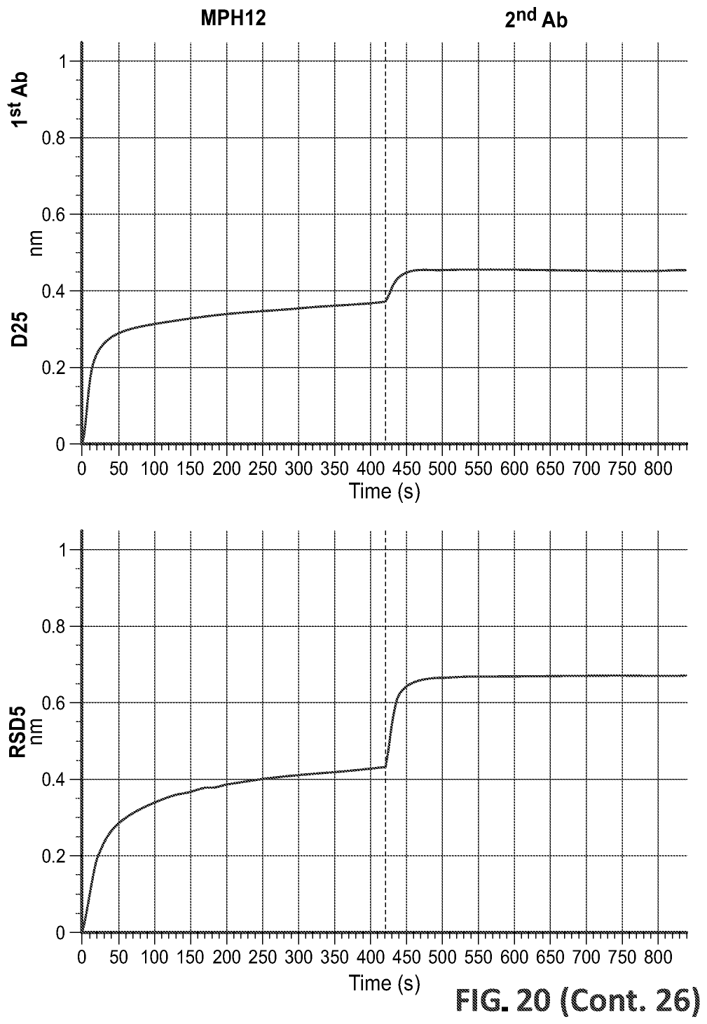

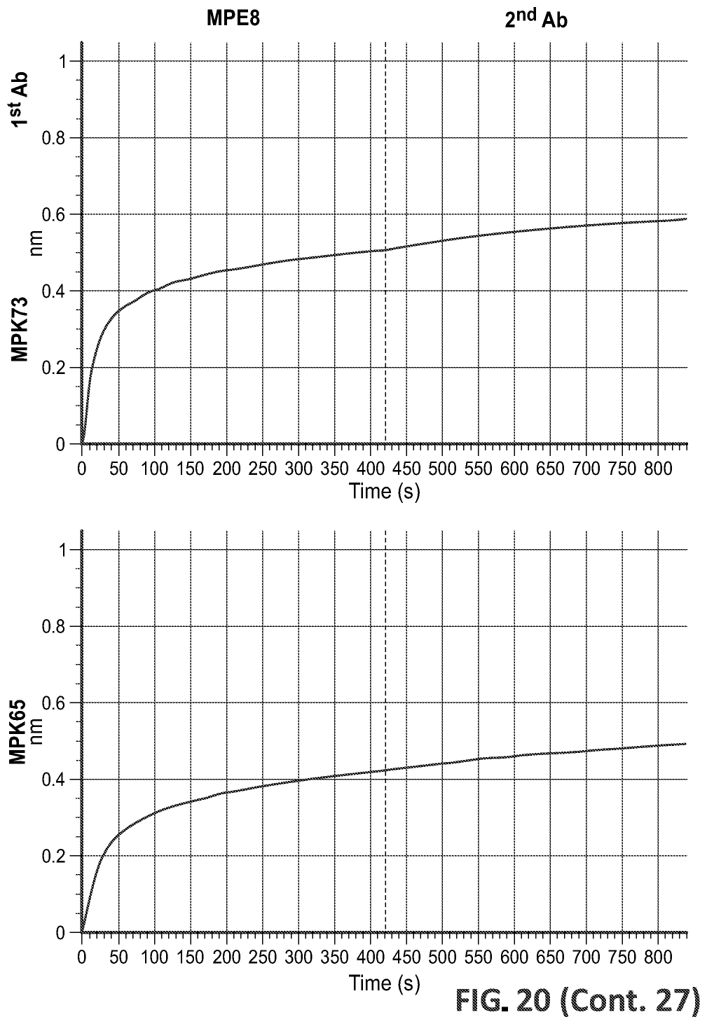

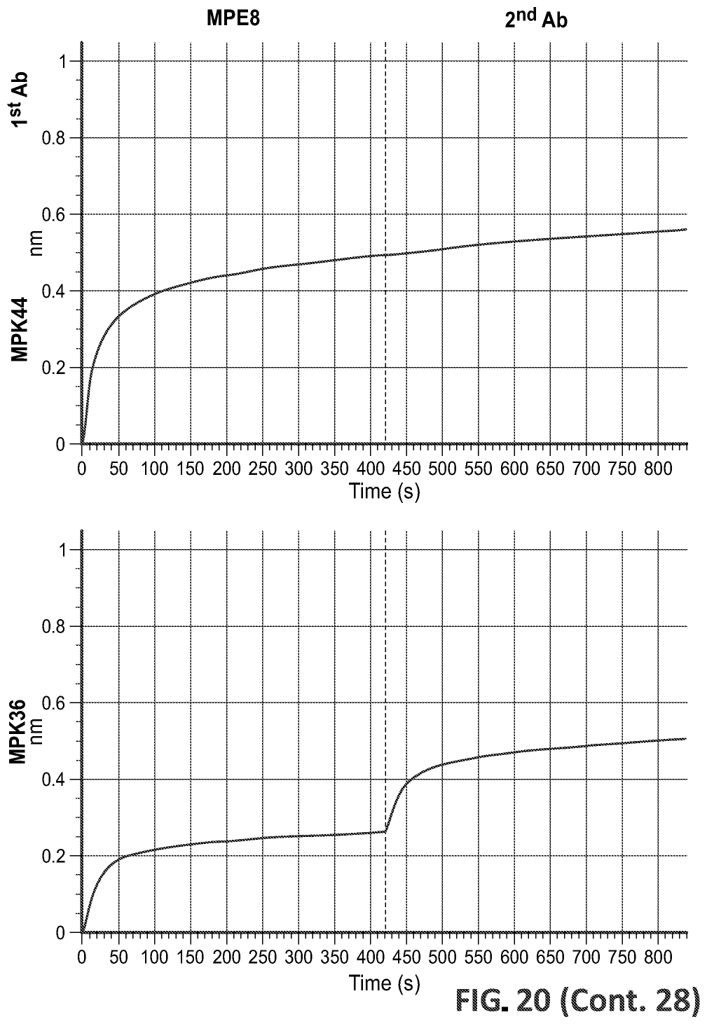

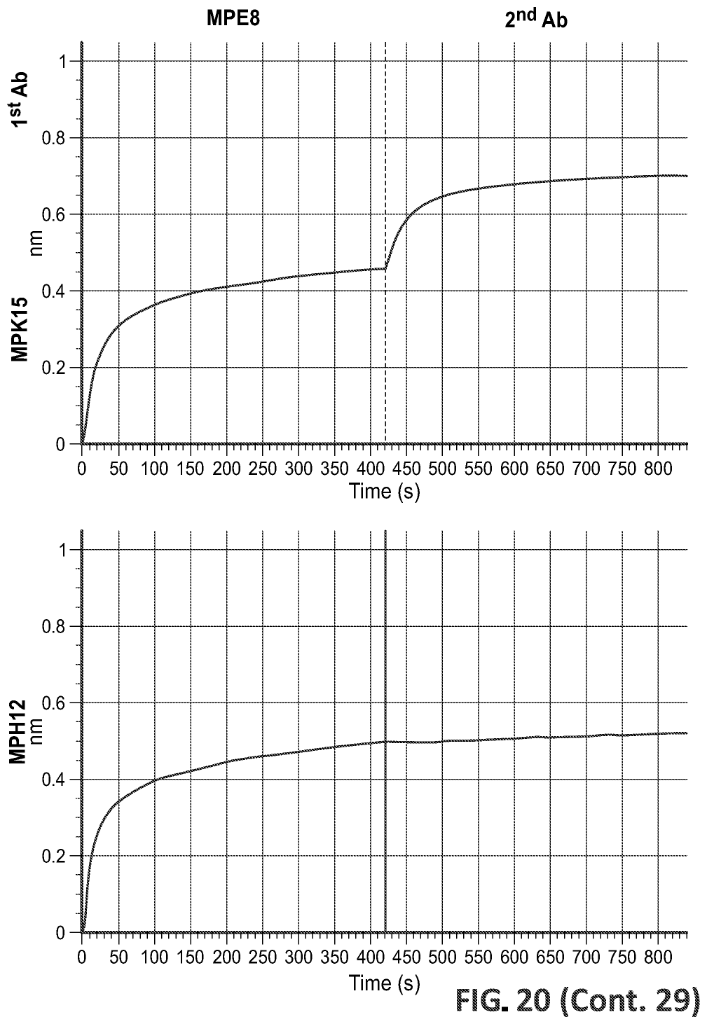

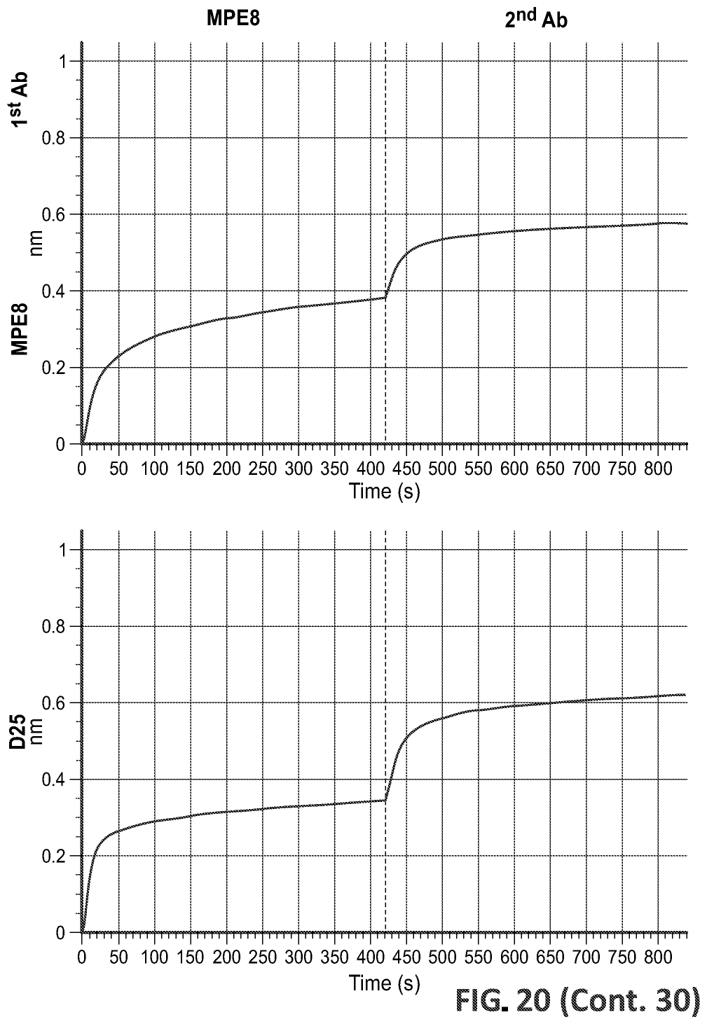

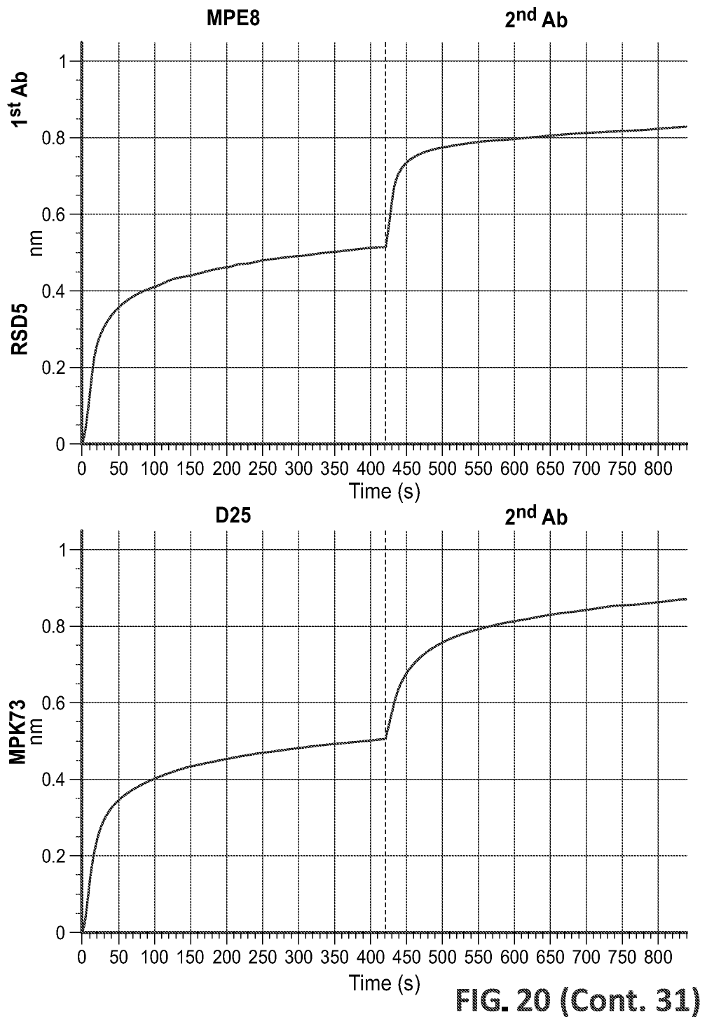

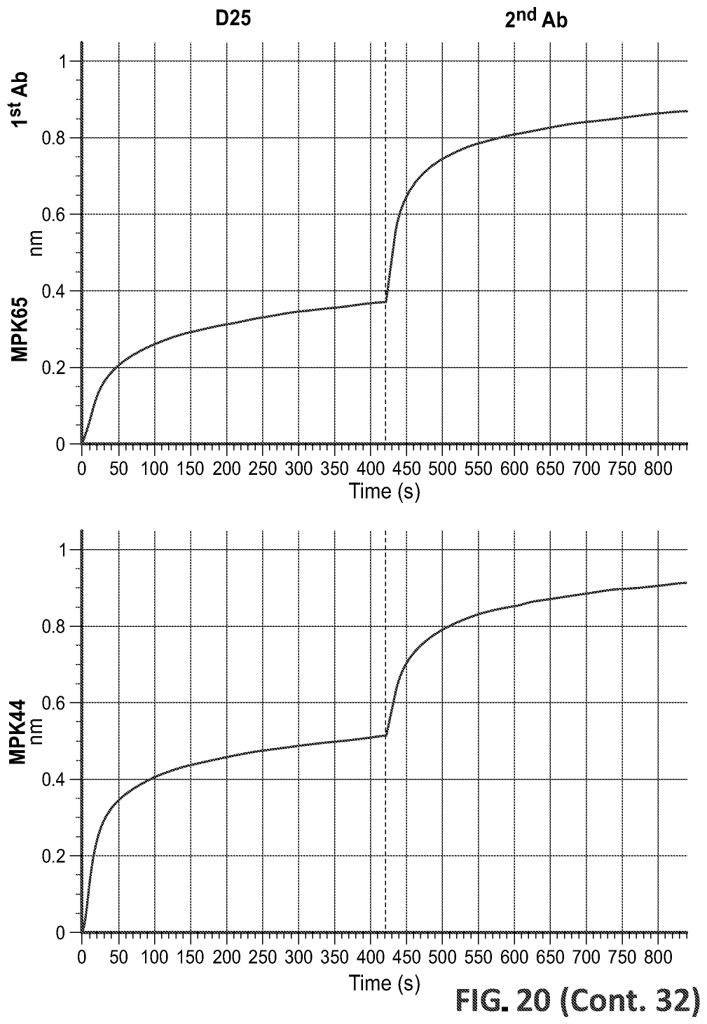

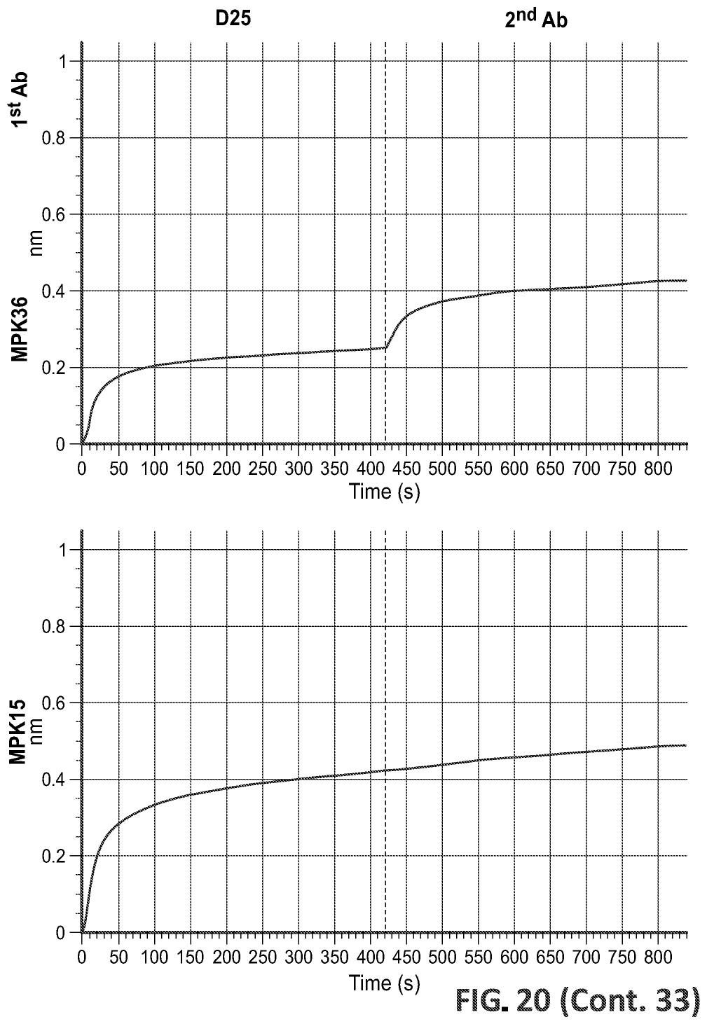

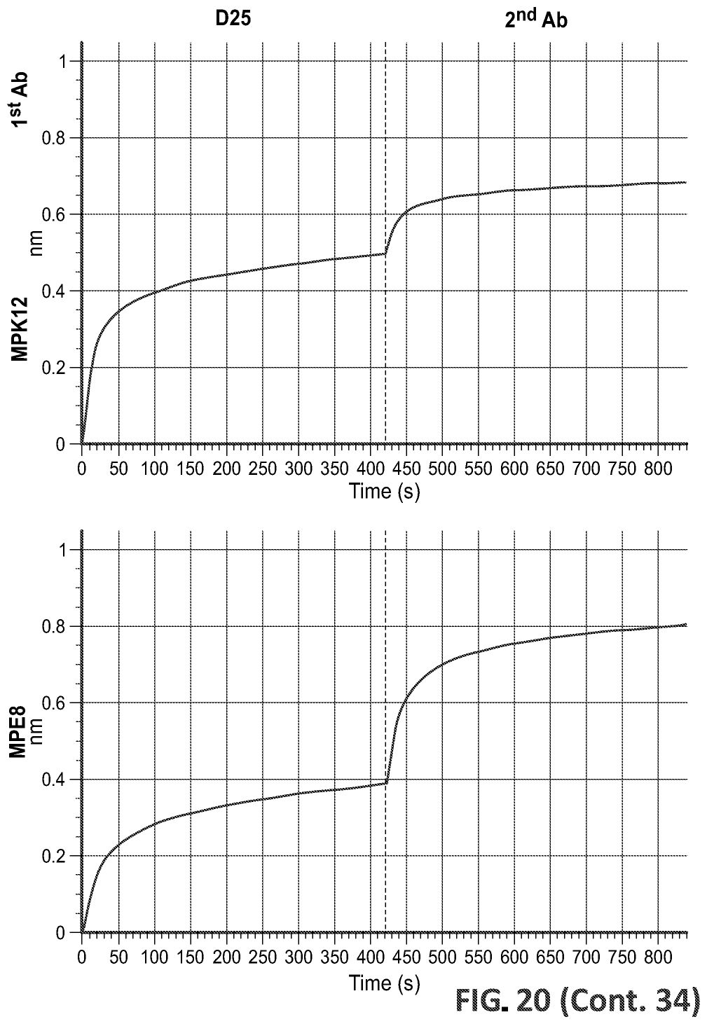

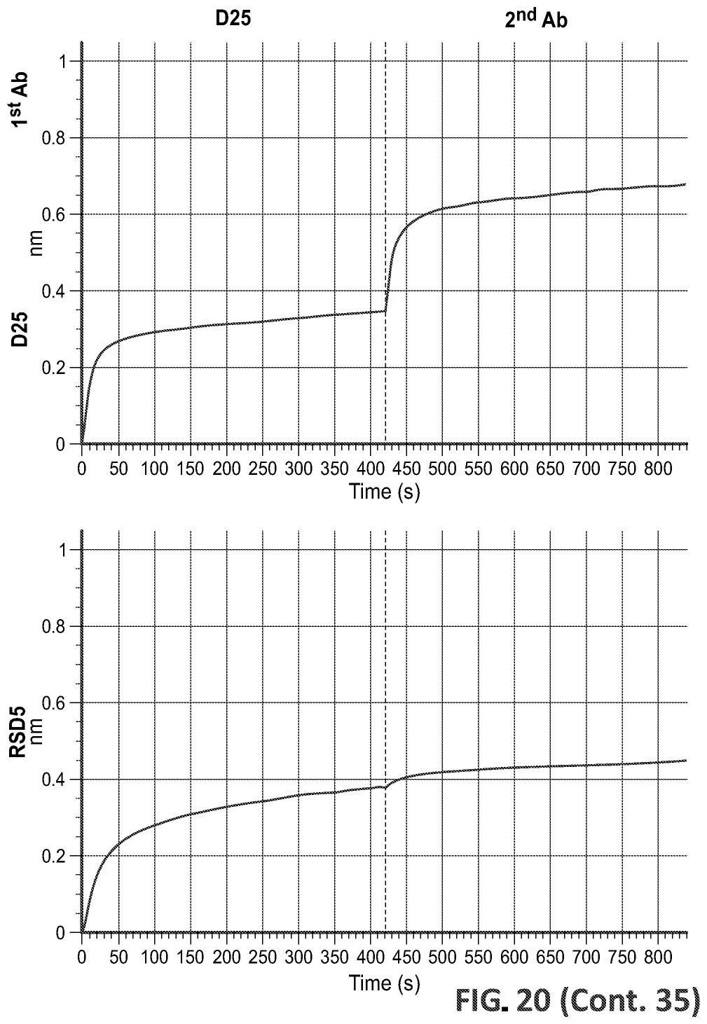

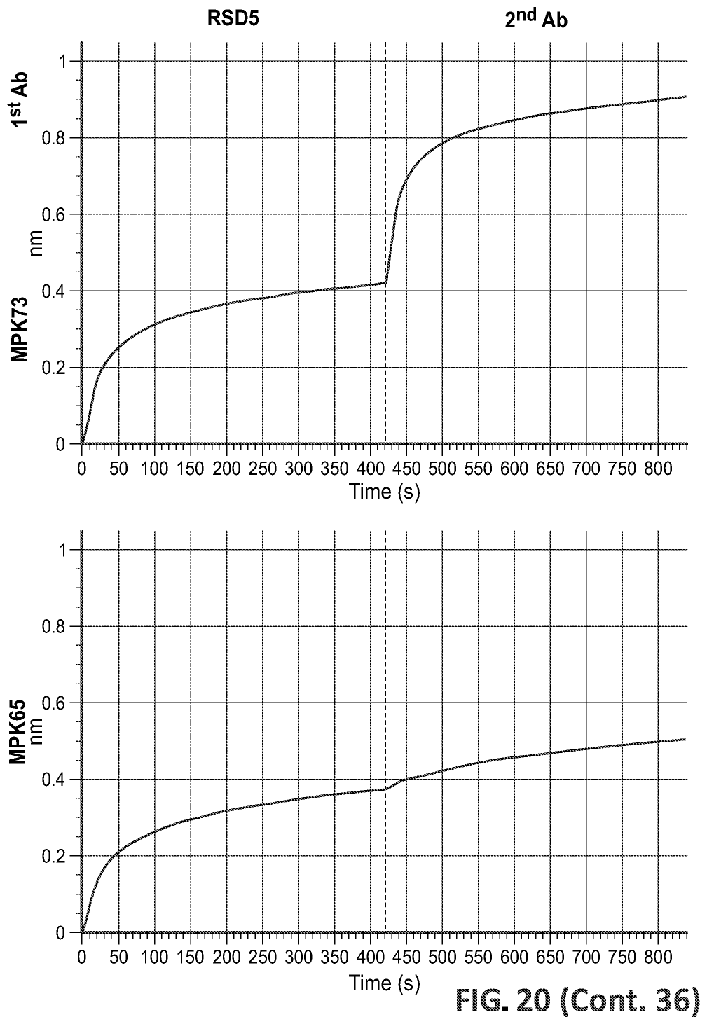

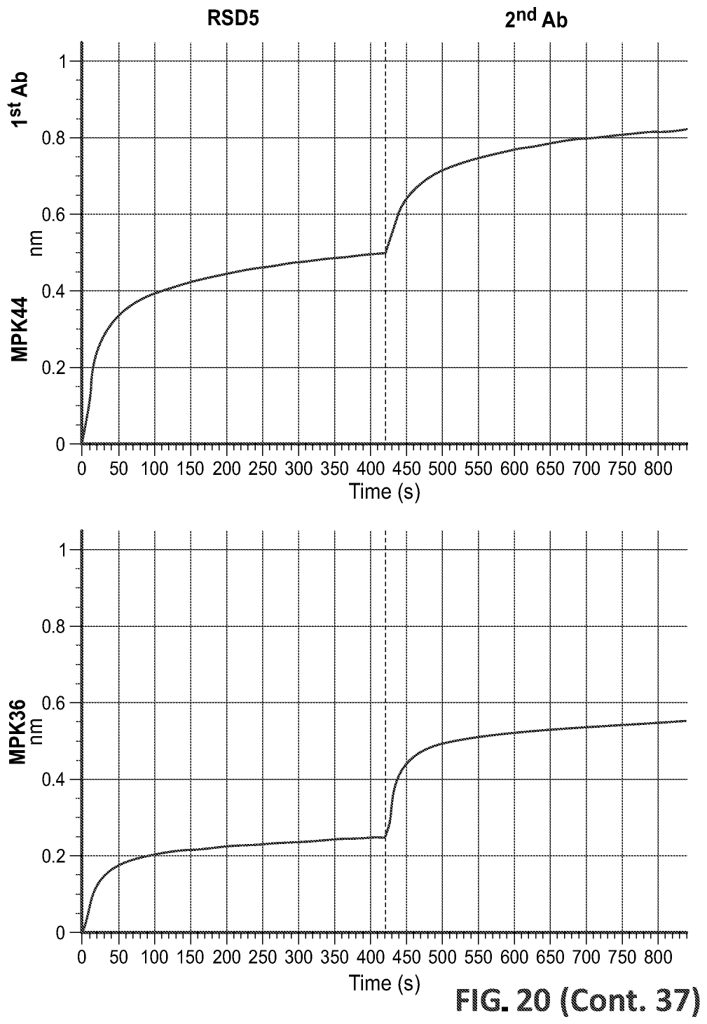

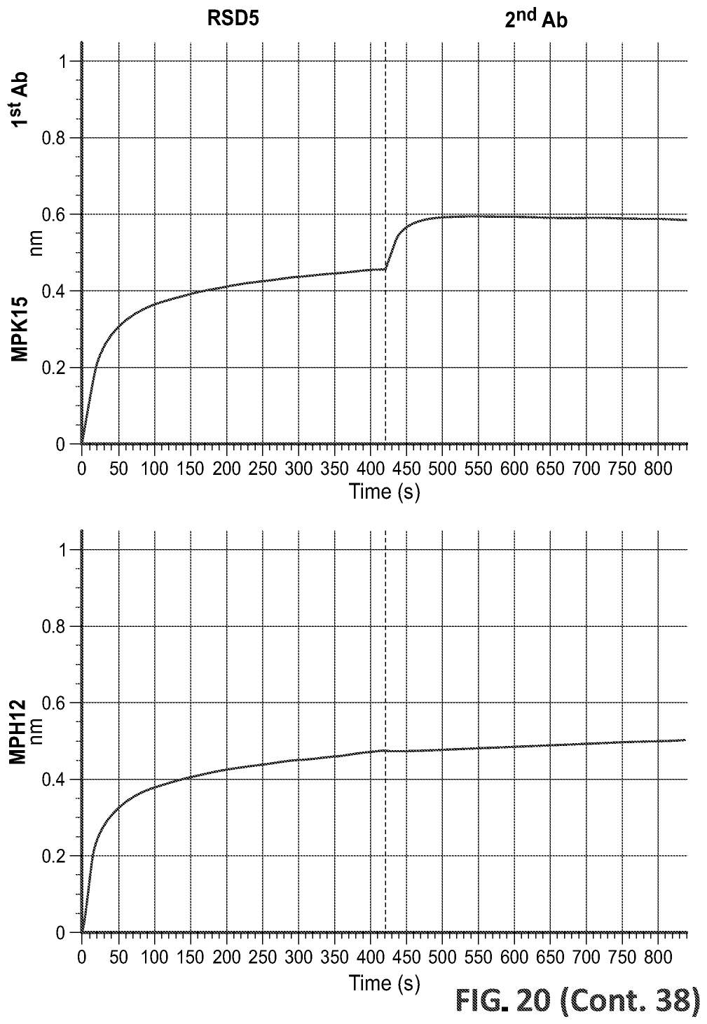

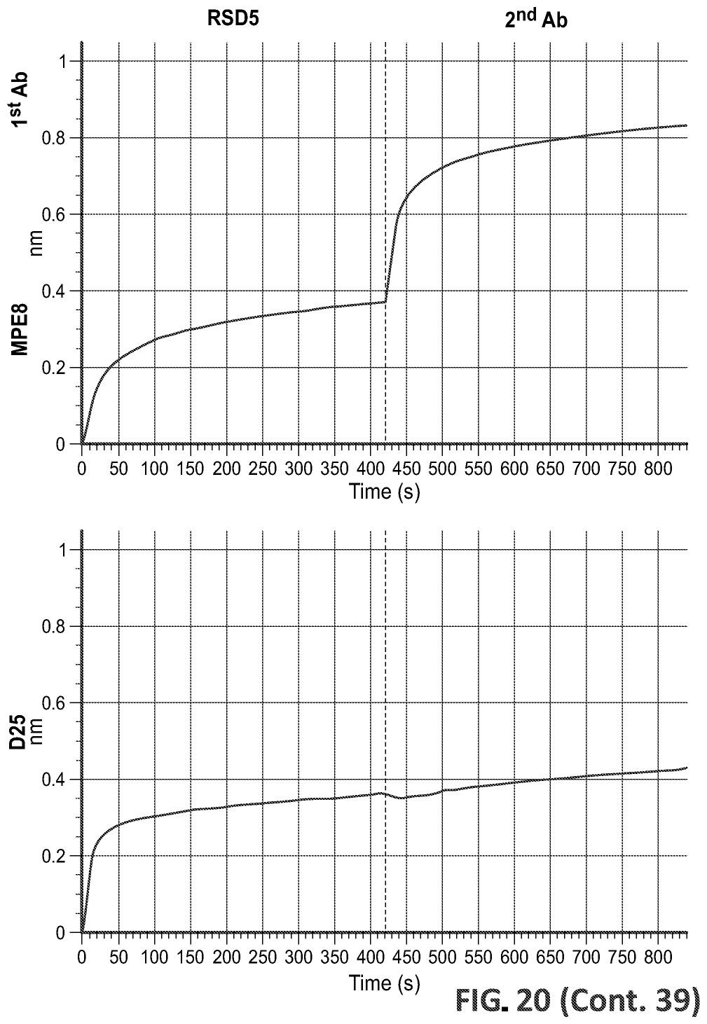

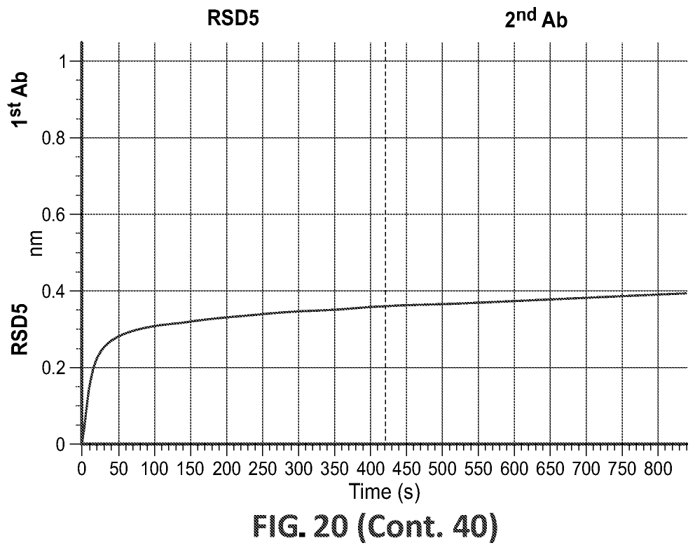

MPK73, MPK65, MPK 44, MPK36, MPK15, and MPH12 were tested with comparator antibodies MPE8, D25 and RSD5. Results are presented in FIG. 20.

MPK65 and MPK44 compete with one another and with D25 (parental mAb of nirsevimab) and RSD5, suggesting that they recognize the same epitope. MPH12, MPE8, MPK36 and MPK73 compete with one another and do not compete with D25 (parental mAb of nirsevimab) suggesting that they recognize the same epitope.

### Example 13

### MPK Mabs Induce Antibody-Dependent Cytotoxicity (ADCC)

The ability of mAbs to induce ADCC was assayed as follows:

Preparation of Human NK cells from whole blood: NK cells were freshly isolated from whole EDTA blood using the MACSxpress NK isolation Kit following the manufacturer instruction. Briefly, anticoagulated blood is mixed in a 50 ml tube with 15 ml of the NK isolation cocktail and incubated for 5 minutes at room temperature using a rotator at approximately 12 round per minute. The tube is then placed in the magnetic field of the MACSxpress Separator for 15 minutes. The magnetically labeled cells will adhere to the wall of the tube while the aggregated erythrocytes sediment to the bottom. The target NK cells are then collected from the supernatant while the tube is still inside the MACSxpress Separator. NK cells are centrifuged, treated with distilled water to remove residual erythrocytes, centrifuged again and finally resuspended in AIM-V medium.

Determination of Antibody-Dependent NK cell killing: MAbs were serially diluted 10-fold in AIM-V medium. Target cells (Hep2 infected with RSV A2) were added in a round bottom 384-well plate at 7.5×103 cells/well in 23 μl, then serially diluted antibodies were added to each well (23 μl per well), and the antibody/cell mixture was incubated for 10 minutes at room temperature. After incubation, human NK cells were added at a cell density of 7.5×104/well in 23 μl, yielding an effector to target ratio of 10:1. Control wells were also included that were used to measure maximal lysis (containing target cells with 23 μl of 3% Triton x-100) and spontaneous lysis (containing target cells and effector cells without antibody). Plates were incubated for 4 hours at 37° C. with 5% C02. Cell death was determined by measuring lactate dehydrogenase (LDH) release using a LDH detection kit according to the manufacturer's instructions. In brief, plates were centrifuged for 4 minutes at 400× g, and 35 μl of supernatant was transferred to a flat 384-well plate. LDH reagent was prepared and 35 μl were added to each well. Using a kinetic protocol, the absorbance at 490 nm and 650 nm was measured once every 2 minutes for 8 minutes. The percent specific lysis was determined by applying the following formula: (specific release−spontaneous release)/(maximum release−spontaneous release)×100.

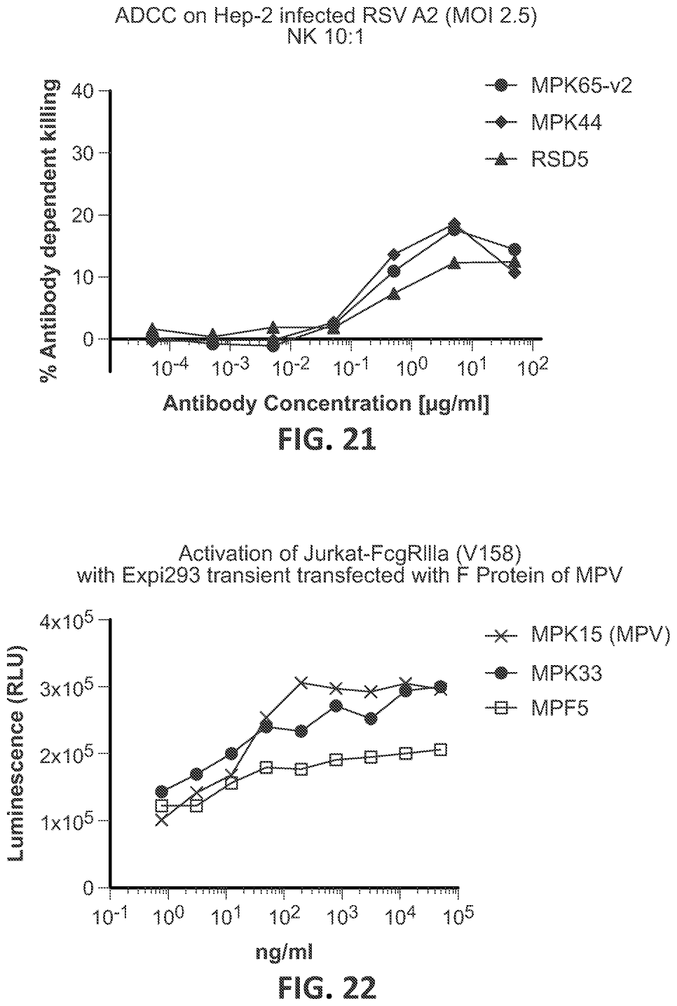

Two mAbs (MPK44 and MPK65-v2), as shown in FIG. 21, showed poor, but comparable levels of ADCC.

### Experiment 2

Experiment 2 was carried out in the same manner as Experiment 1, except that target cells were 293Expi transfected with MPV F D280 protein (from MPV_NL/1/99). Results are shown in FIG. 22. MPK15 induced strong activation of FcgRIIIA.

### Experiment 3

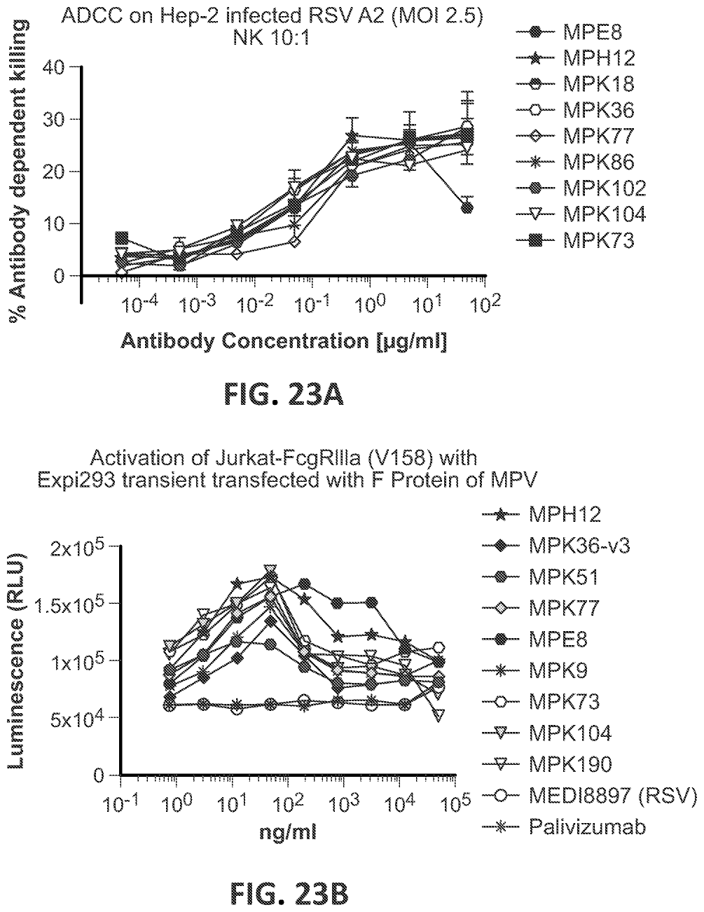

Experiment 3 was carried out in the same manner as Experiment 1, except that target cells were 293Expi transfected with RSV F protein or 293Expi transfected with MPV F D280 protein (from MPV_NL/1/99). Various mAbs, as shown in FIG. 23A and FIG. 23B, showed comparable levels of ADCC in RSV-transfected and MVP-transfected cells.

### Example 14

### MPK mAbs Induce Activation of Human FcgRIIIa and FcgRIIa

The ability of various mAbs to induce activation of Human FcgRIIIa and ADCP was assayed as follows:

### Experiment 1

MAbs were serially diluted 4-fold in ADCP Assay buffer. Target cells (293Expi transfected with RSV F protein) were added in a white flat bottom 96-well plate at 12.5×103 cells/well in 25 μl, then serially diluted antibodies were added to each well (25 μl per well), and the antibody/cell mixture was incubated for 25 minutes at room temperature. Effector cells for the ADCP Bioassay are thawed and added at a cell density of 7.5×10⁴/well in 25 μl, yielding an effector to target ratio of 6:1. Control wells were also included that were used to measure antibody-independent activation (containing target cells and effector cells but no antibody) and spontaneous luminescence of the plate (wells containing the ADCP Assay buffer only). Plates were incubated for 23 hours at 37° C. with 5% C02. Activation of human FcγRIIIa (V158 variant) in this bioassay results in the NFAT-mediated expression of the luciferase reporter gene. Luminescence is therefore measured with a luminometer using the Bio-Glo-TM Luciferase Assay Reagent according to the manufacturer's instructions. The data (i.e. specific FcgRIIIa activation) are expressed as the average of relative luminescence units (RLU) over the background by applying the following formula: (RLU at concentration× of mAb−RLU of background).

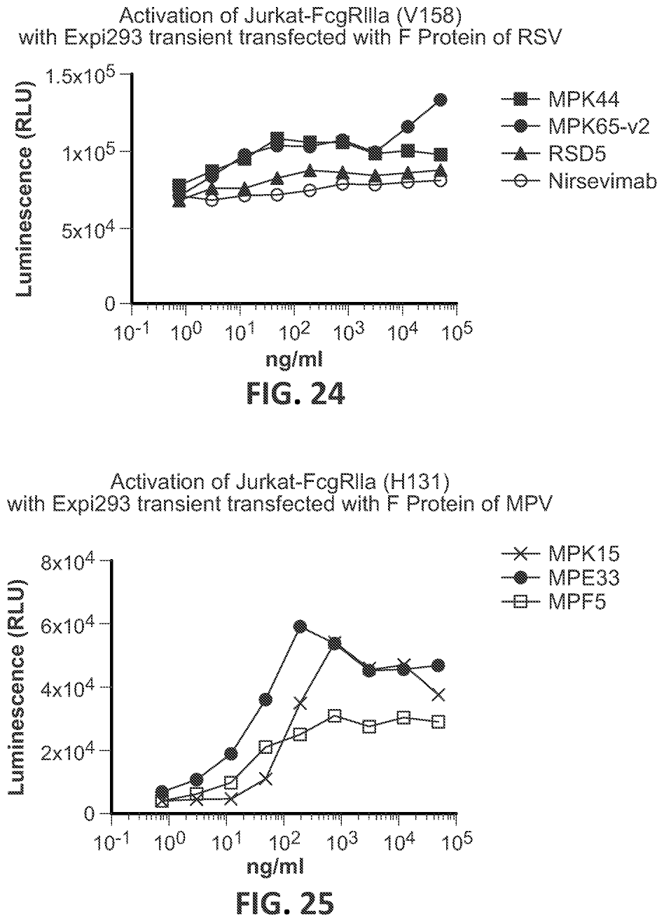

MPK44 and MPK65-v2 showed poor, but comparable levels of FcgRIIIA activation (FIG. 24). Nisevimab and palivizumab did not activate FcgRIIIA.

In this and other Examples, nisevimab unless otherwise indicated, may refer to an antibody containing the VH and VL of nirsevimab undergoing clinical trials, including antibodies containing a different Fc portion than nirsevimab undergoing clinical trials, as such antibodies are expected to retain antigen-binding characteristics of nisevimab.

### Experiment 2

Experiment 2 was carried out in the same manner as Experiment 1, except that target cells were 293Expi transfected with MPV F D280 protein (from MPV_NL/1/99). Results are shown in FIG. 25. MPK15 showed strong activation of FcgRIIA.

### Experiment 3

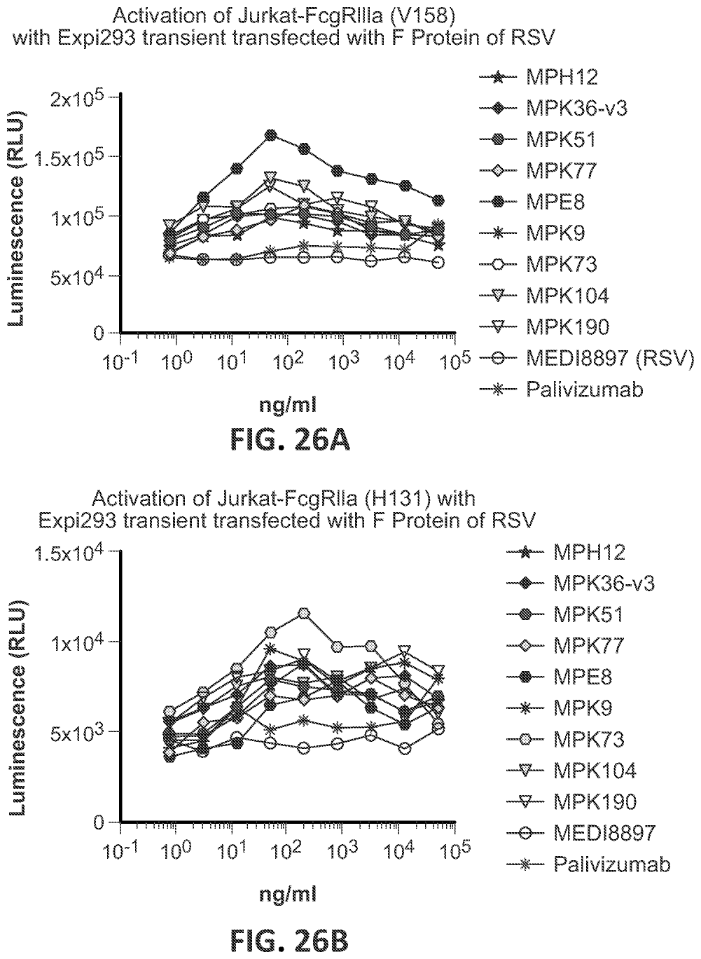

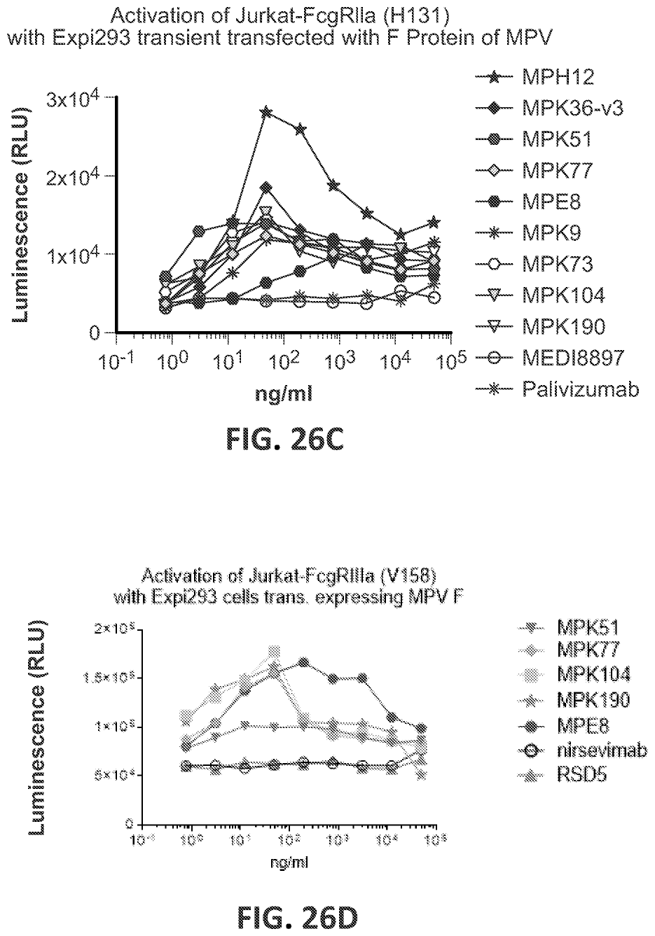

Experiment 3 was carried out in the same manner as Experiment 1, except that target cells were 293Expi transfected with RSV F protein or 293Expi transfected with MPV F D280 protein (from MPV_NL/1/99). Results are shown in FIG. 26A and FIG. 26B (RSV) and FIG. 26C and FIG. 26D (MPV), most of the selected RSV-MPV mAbs showed strong activation of FcgRIIIA, both on RSV and on MPV transfected cells. Nisevimab and palivizumab did not activate FcgRIIIA.

### Example 15

### Resistance to Generation of RSV Escape Mutants

Resistance of mAbs to the generation of RSV escape mutations was assayed as follows:

Material: Infection medium (IM): MEM 2.4% Hyclone+P/S

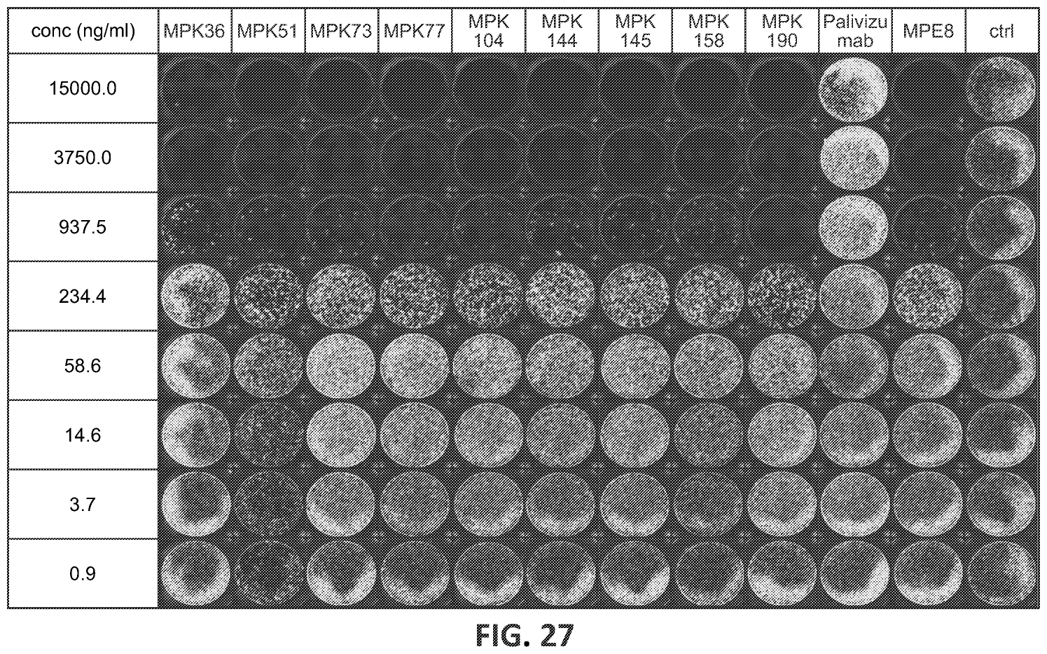

Procedure—day 0: Transfer 60 ul virus from previous step SN in a new plate. For each mAb titration and using a multichannel pipet vertically, take 20 ul from the wells B-H, move up by one well and pipet out (rows A-G); Finally add 20 ul IM in well H; Do serial dilution of the mAb in IM and add 60 ul mAb 4× per well (starting final concentration is 3 μg/ml→1:4 serial dilutions); Incubate 45 min at 37° C.; In the meanwhile detach HEp-2 cells and count them; dilute cells to have to have 12,000 cells in 120 μl IM and add suspension in each well; Incubate until cytophatic effects occurs (3-4 days); day 3—Read the plate at the Cytation 5 (threshold on green signal→plot area of green objects); Collect SN by pipetting up and down on the cellular monolayer and transfer it in a 96 wp round bottom; centrifuge at 1000× g for 5 min to remove cellular debris; Transfer 60 ul of the serial dilutions to a new culture plate for next step, if possible do not freeze to avoid virus loss; Transfer 60 ul SN in a 96 wp V-bottom (see products) containing 60 ul Sucrose 50% and mix (backup plate); Seal plate with CAP MAT (see products); Put backup plate at −80° C.; Perform 8 cycles of re-infection to assess generation of escape mutants. Results are presented in FIG. 27. MPK mAbs did not select RSV escape mutants, unlike palivizumab (Pali).

### Example 16

### Resistance to Generation of MPV Escape Mutants

Resistance of mAbs to the generation of RSV escape mutations was assayed as follows:

Material: Infection medium (IM): MEM 2.4% Hyclone+P/S.

Procedure—day 0: Transfer 30 μl virus from previous step SN in a new plate. For each mAb titration and using a multichannel pipet vertically, take 10 μl from the wells B-H, move up by one well and pipet out (rows A-G); Finally add 10 μl IM in well H; Do serial dilution of the mAb in IM and add 60 ul mAb 4× per well (starting final concentration is 3 μg/ml→1:4 serial dilutions); Incubate 45 min at 37° C.; In the meanwhile detach LLC-MK2 cells and count them; dilute cells to have to have 10,000 cells in 120 μl IM and add suspension in each well; —day 1: Add 30 μl/well TPCK treated trypsin 8× in IM (360 ug/ml→final concentration is 45 μg/ml); Incubate until cytophatic effects occurs (6 additional days in order to do 1 reinfection step per week without freezing the virus input material for the next step); —day 6: Read the plate at the Cytation 5 (threshold on green signal→plot area of green objects); Add 2.4 μl/well HEPES 1M to a final concentration of 10 mM as a preservative; Collect SN by pipetting up and down on the cellular monolayer and transfer it in a 96 wp round bottom; centrifuge at 1000× g for 5 min to remove cellular debris; Transfer 100 ul SN in a 96 wp (backup plate), seal plate with CAP MATV-bottom and store at −80° C.; Transfer 50 μl of the following well (highlighted in red) in a 12 wp for putative MARMs propagation; Perform 8 cycles of re-infection to assess generation of escape mutants.

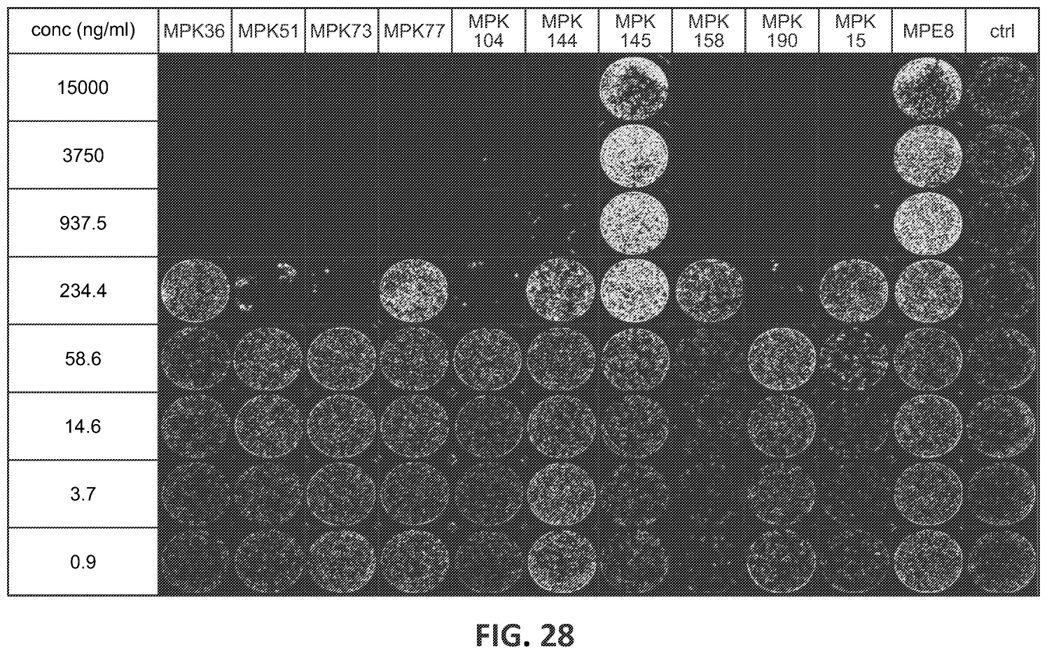

Results for various mAbs are in FIG. 28. MPE8 and MPK145 isolate full escape mutants (antibodies potency loss is >64 fold). MPK77 and MPK144 isolate partial escape mutants (antibody potency loss is ˜4 fold). MPK104 seems to isolate an escape mutant that however showed a very slow replication. MPK36, MPK51, MPK73, MPK158 and MPK190 did not isolate escape mutants. MPK15 also did not isolate escape mutants (data now shown).

### Example 17

### In Vivo Testing

MPK190-rIgG1-LS (an IgG1 with the LS mutation), MPK51-rIgG1-LS, MPK77-rIgG1-LS, MPK 104-rIgG1-LS, or positive control antibody nirsevimab were administered to separate test groups of female BALB/c mice, ages 7-9 weeks, at doses of 1.0 or 0.5 mg/kg. One test group was administered 1 mg/kg isotype control antibody FNI17-rIgG1-LS, and another test group was administered 10 μL/g vehicle (1×PBS). Each group contained five mice, except the nirsevimab 0.5 mg/kg group, which contained 4 mice. Administration was via the tail vein and tool place on Day−1 of the study. Injection volume as 10 μL/g in 1×PBS.

On Day 0 of the study, mice were infected with 3E6pfu/mouse of RSV (the mouse adapted D6 clone MaRSV P3 28 Oct. 2021 1.18E8pfu/mL). Blood was collected via cheek bleed.

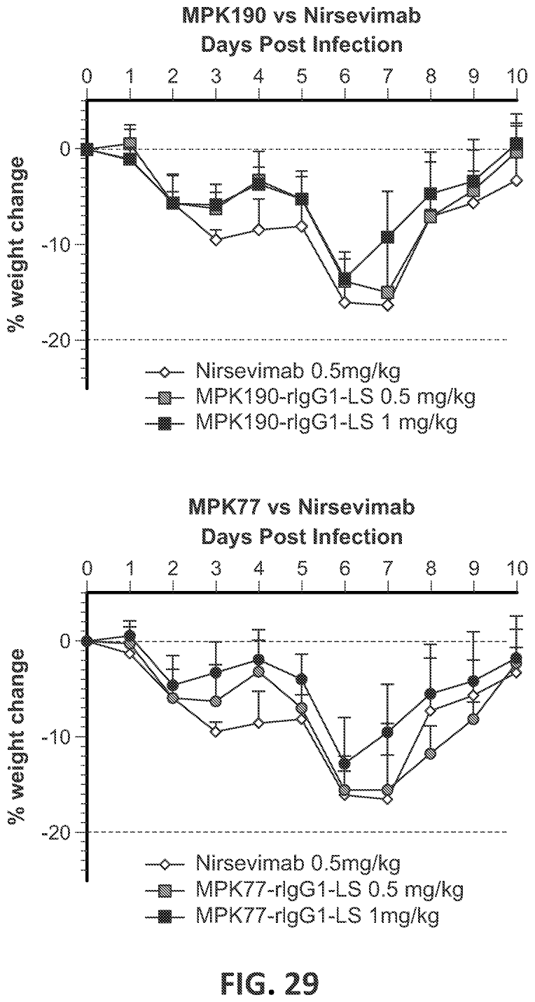

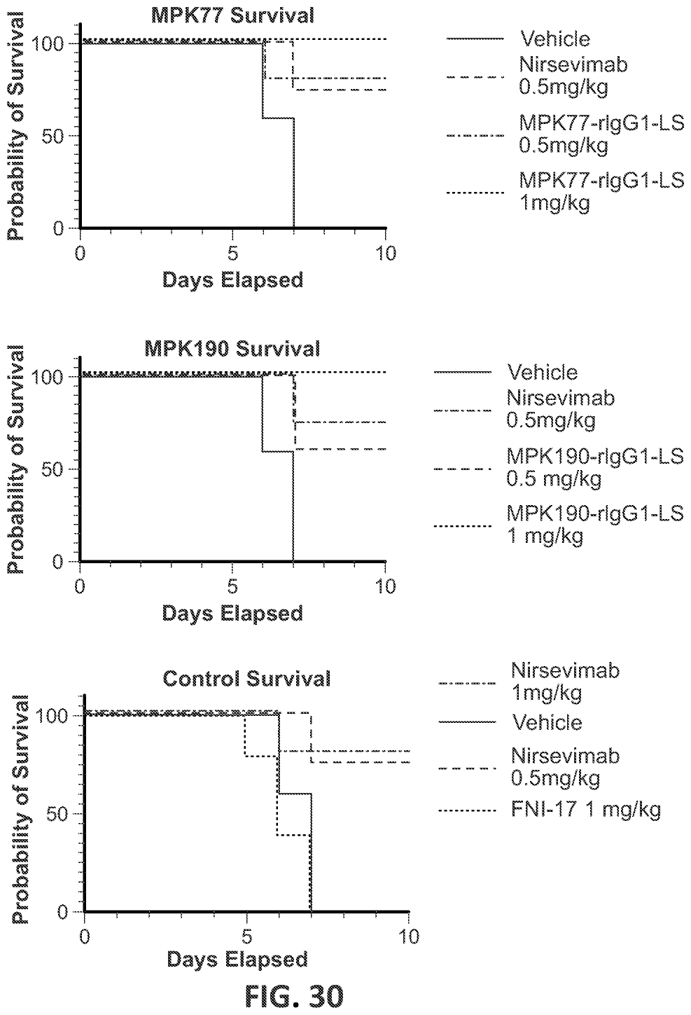

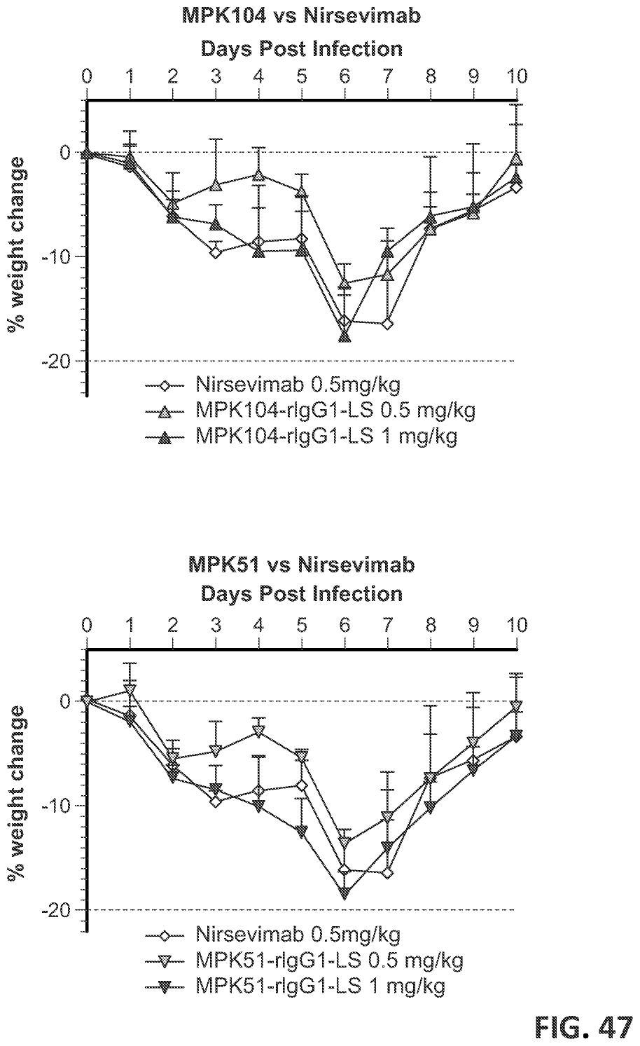

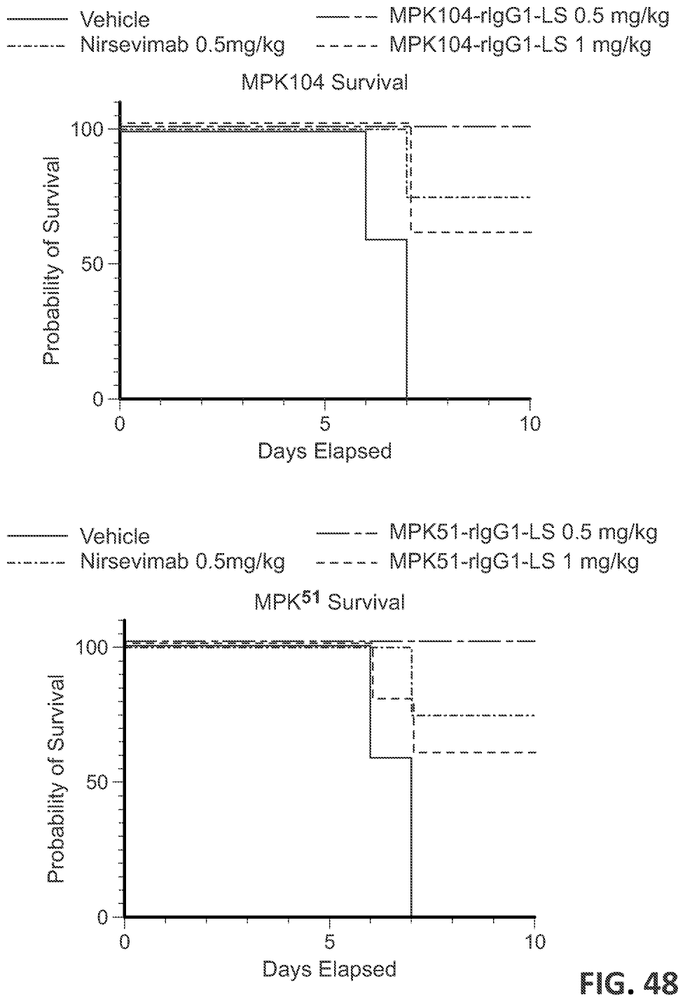

IC50 graphs of neutralization of the mouse adapted RSV clone by MPK190, MPK104, MPK51, MPK77, and MEDI8897 in the RSV-infected mice are shown in FIG. 46. Weight loss and morbidity were monitored on Days 1-10, with an 80% body weight cutoff. Results for MPK190 and MPK77 are presented in FIG. 29 (weight loss) and FIG. 30 (morbidity) and show positive results. Results for MPK104 and MPK51 are presented in FIG. 47 (weight loss) and FIG. 48 (morbidity).

The pharmacokinetics of representative antibodies was evaluated using standard methods. Results are provided in Table 26.

TABLE 26
PK Data

| | | | | | | | | | |
|---|---|---|---|---|---|---|---|---|---|
| | | | El. | Half- | AUClast | | CLss | | AUClast_ D |
| Dose | | | Const. | life | day*ug/ | Cavg | ml/ | Vz | day*ug/ |
| ug | Rsq | Points | 1/day | day | ml | ug/ml | day | ml | ml/ug |

MPK77

| | | | | | | | | | | |
|---|---|---|---|---|---|---|---|---|---|---|
| | 110 | 0.99 | 13 | 0.04 | 15.83 | 583.04 | 42.4 | 2.59 | 59.25 | 5.3 |
| | 95 | 0.99 | 13 | 0.05 | 13.84 | 594.78 | 50.08 | 1.9 | 37.88 | 6.26 |
| | 110 | 0.98 | 13 | 0.05 | 15.31 | 637.68 | 47.55 | 2.31 | 51.09 | 5.8 |
| | 100 | 0.98 | 13 | 0.04 | 16.6 | 729.41 | 50.47 | 1.98 | 47.44 | 7.29 |
| | 105 | 0.99 | 13 | 0.04 | 16.6 | 722.19 | 52.14 | 2.01 | 48.22 | 6.88 |
| Mean | 104 | 0.98 | 13 | 0.045 | 15.64 | 653.42 | 48.53 | 2.16 | 48.777 | 6.306 |
| SD | 6.519 | 0.01 | 0 | 0.003 | 1.14 | 69.183 | 3.801 | 0.289 | 7.681 | 0.803 |

MPK 104

| | | | | | | | | | | |
|---|---|---|---|---|---|---|---|---|---|---|
| | 95 | 0.99 | 13 | 0.06 | 10.69 | 570.69 | 52.08 | 1.82 | 28.13 | 6.01 |
| | 105 | 0.98 | 13 | 0.05 | 12.68 | 663.83 | 63.92 | 1.64 | 30.05 | 6.32 |
| | 110 | 0.99 | 13 | 0.06 | 10.87 | 655.38 | 63.01 | 1.75 | 27.37 | 5.96 |
| | 105 | 0.96 | 13 | 0.05 | 13.35 | 591.56 | 57.74 | 1.82 | 35.01 | 5.63 |
| | 100 | 0.99 | 13 | 0.05 | 12.82 | 631.31 | 45.84 | 2.18 | 40.34 | 6.31 |
| Mean | 103 | 0.98 | 13 | 0.058 | 12.08 | 622.555 | 56.516 | 1.843 | 32.181 | 6.047 |
| SD | 5.701 | 0.01 | 0 | 0.006 | 1.216 | 40.337 | 7.617 | 0.203 | 5.445 | 0.286 |

MPK 51

| | | | | | | | | | | |
|---|---|---|---|---|---|---|---|---|---|---|
| | 119.1 | 0.97 | 13 | 0.05 | 14.01 | 743.184 | 70 | 1.7 | 34.39 | 6.24 |
| | 119.1 | 0.96 | 13 | 0.06 | 11.87 | 678.87 | 77.77 | 1.53 | 26.23 | 5.7 |
| | 125.4 | 0.99 | 13 | 0.05 | 15.17 | 729.828 | 56.19 | 2.23 | 48.85 | 5.82 |
| | 112.9 | 0.98 | 13 | 0.05 | 13.59 | 434.665 | 56.84 | 1.99 | 38.94 | 3.85 |
| | 112.9 | 0.98 | 13 | 0.05 | 14.96 | 724.818 | 60.81 | 1.86 | 40.06 | 6.42 |
| Mean | 117.88 | 0.97 | 13 | 0.05 | 13.92 | 662.273 | 64.322 | 1.861 | 37.695 | 5.605 |
| | 5.223 | 0.01 | 0 | 0.005 | 1.319 | 129.521 | 9.322 | 0.268 | 8.276 | 1.024 |

MPK 190

| | | | | | | | | | | |
|---|---|---|---|---|---|---|---|---|---|---|
| | 100 | 1 | 13 | 0.05 | 14.59 | 611 | 47.11 | 2.12 | 44.67 | 6.11 |
| | 85 | 0.97 | 13 | 0.04 | 15.42 | 655.35 | 56.32 | 1.51 | 33.57 | 7.71 |
| | 100 | 0.99 | 13 | 0.05 | 13.65 | 561 | 45.68 | 2.19 | 43.1 | 5.61 |
| | 85 | 0.97 | 13 | 0.06 | 12.38 | 607.75 | 68.75 | 1.24 | 22.08 | 7.15 |
| | 100 | 0.99 | 13 | 0.05 | 15.22 | 597 | 45.23 | 2.21 | 48.55 | 5.97 |
| Mean | 94 | 0.98 | 13 | 0.049 | 14.25 | 606.42 | 52.62 | 1.854 | 38.394 | 6.51 |
| SD | 8.216 | 0.01 | 0 | 0.005 | 1.254 | 33.796 | 10.086 | 0.451 | 10.656 | 0.881 |

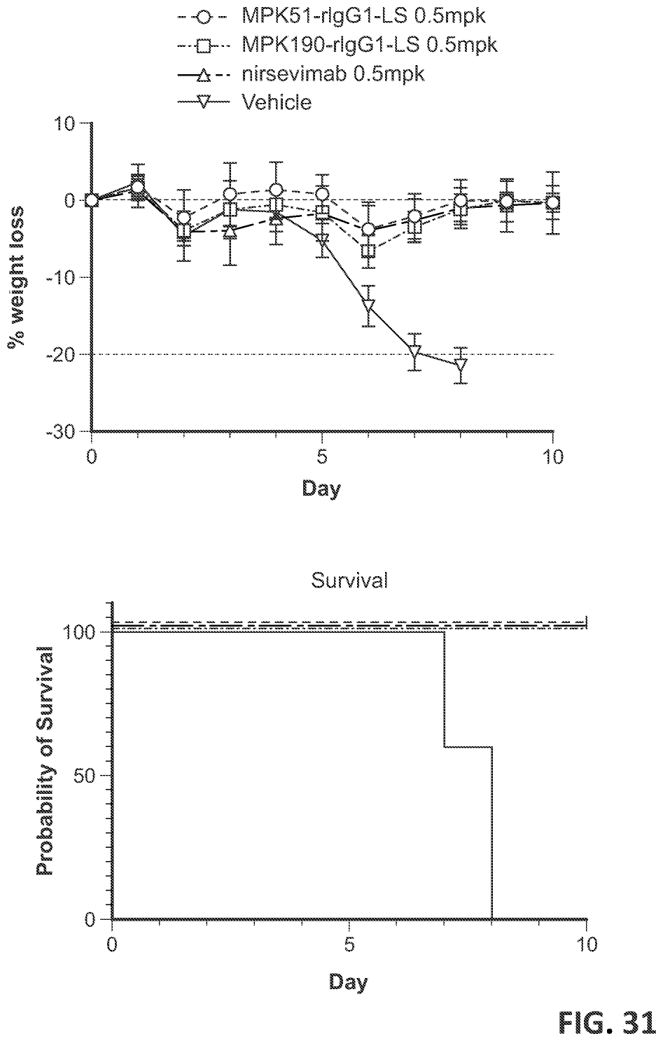

A similar test was conducted using 0.5 mg/kg of MPK190-rIgG1-LS, MPK51-rIgG1-LS, or positive control antibody nirsevimab in 10 week old BALB/c mice. Results are presented in FIG. 31.

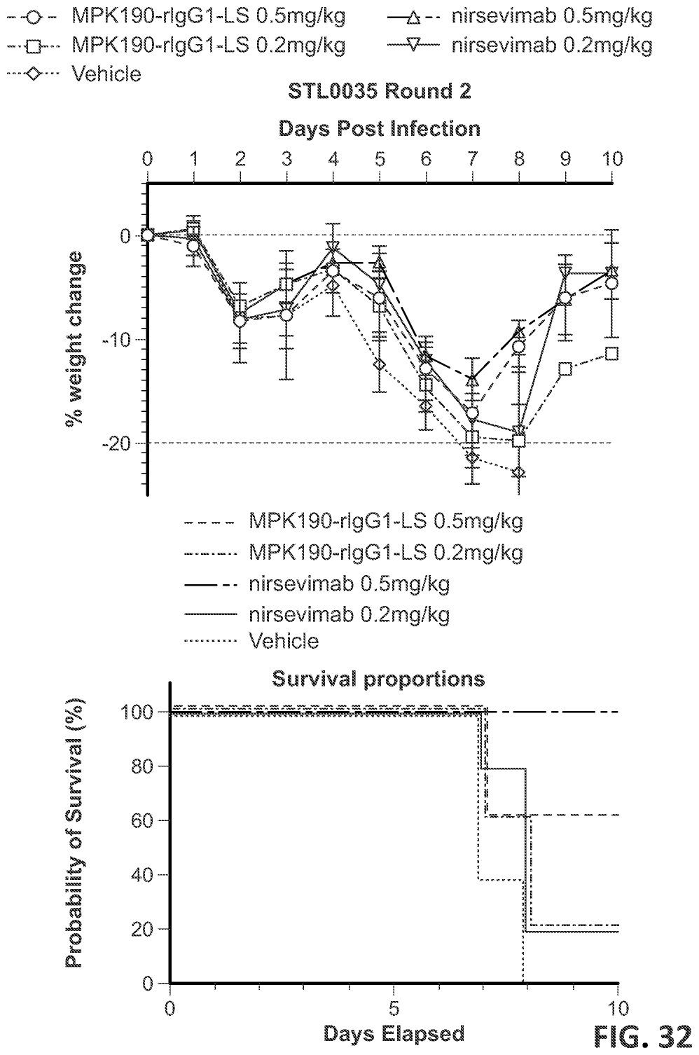

Yet another test was conducted including low doses (0.2 mg/kg) or MPK190-rIgG1-LS or positive control antibody nirsevimab in 8-9 week old BALB/c mice. Results are presented in FIG. 32.

### Example 18

### RSV-MPV Dual Mabs Binding to 11 RSV A and 8 RSV B Strains

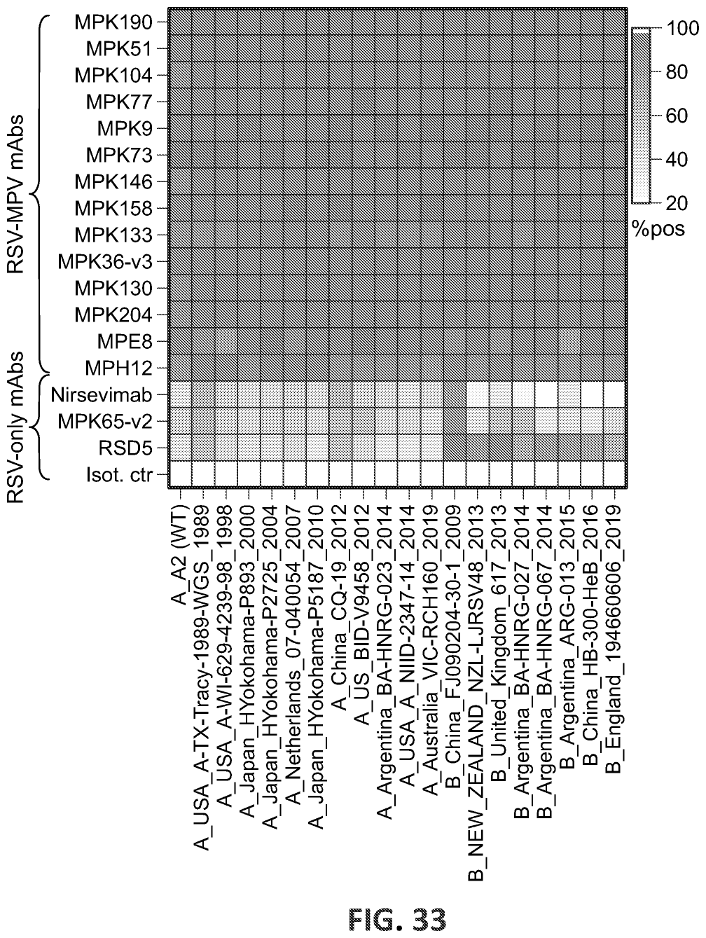

A RSV and MPV database was created from 4,515 RSV F sequences (GISAID) (2,714 RSV A+1,801 RSV B) and 1,783 RSV F sequences (NCBI). 19 RSV F unique sequences representative of circulating strains were selected to increase protein distance and representation of strains over time. Expi293 F cells were transiently transfected with plasmids encoding for the representative RSV F proteins. FIG. 33 shows a heat map of variant antibodies binding to RSV A and RSV B strains as assessed by fluorescence-activated cell sorting (FACS). All the RSV-MPV dual mAbs showed broad binding to 11 RSV A and 8 RSV B strains. Some RSV-only mAbs, including nirsevimab, showed low-to-very low binding to several RSV strains.

### Example 19

### RSV-MPV Dual mAbs Binding to MPV Strains

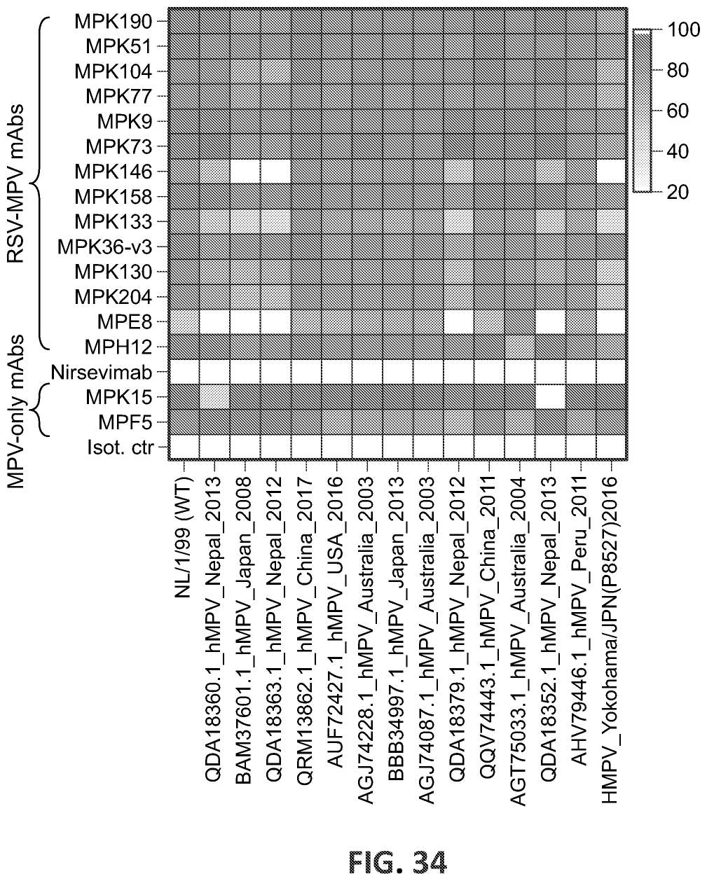

A MPV database was created from 870 MPV F sequences (NCBI). 14 MPV F unique sequences representative of circulating strains were selected to increase protein distance and representation of strains over time. Expi293 F cells were transiently transfected with plasmids encoding for the representative MPV F proteins. FIG. 34 shows a heat map of variant antibodies binding to MPV strains as assessed by FACS. All the RSV-MPV dual mAbs (except for MPE8) showed broad binding to the 14 MPV strains.

### Example 20

### Neutralization of RSV A and B Strains and MPV A and B Strains by RSV-MPV mAbs

Various RSV-MPV mAbs were tested for cross-neutralization of RSV-A and MPV-A2. MPK190, MPK104, MPK51 and MPK77 showed high neutralization potency of RSV-A (IC50<15 ng/ml), which is about 10-fold higher than nirsevimab (IC50 1.7 ng/ml). MPK190 and MPK104 are clonally related, having the same VDJ gene usage: VH3-21/VL1-40. MPK51 and MPK77 are clonally related, having the same VDJ gene usage: VH3-11/VL1-40. These four mAbs also broadly neutralized different RSV and MPV strains, as shown in Table 27. nirsevimab and MPE8 were used as comparators.

TABLE 27
RSV and MPV Neutralization by RSV-MPV mAbs

| | | RSV-A | RSV-B | MPV-A2 | MPV-B2 |
|---|---|---|---|---|---|
| | mAb | (A2) | (B1) | (CAN97-83) | (TN/96-213) |
| Neutralization | MPK190 | 4.3 | 6.3 | 55.3 | 55.0 |
| (IC50, ng/ml) | MPK104 | 1.3 | 6.0 | 21.5 | 75.4 |
| | MPK51 | 1.9 | 1.9 | 53.7 | 50.6 |
| | MPK77 | 1.3 | 2.3 | 29.4 | 30.5 |
| | nirsevimab | 0.2 | 0.4 | No neutr. | No neutr. |
| | MPE8 | 6.0 | 3.7 | 12.6 | 23.6 |

### Example 21

### In Vitro Analysis of Monoclonal Antibody-Resistant Mutants

Monoclonal antibody-resistant mutants (MARMs) of mAbs MPK190, MPK104, MPK51, and MPK77, were analyzed in vitro. MAbs were incubated with RSV A-GFP or MPV A2-GFP, and added to HEp-2 and LLC-MK2 cells. At each next cycle for a total of 8 cycles, supernatants were transferred and mixed with a new mAb titration, before adding to cells. Virus from the first infected wells of cycle 8 were expanded and titrated.

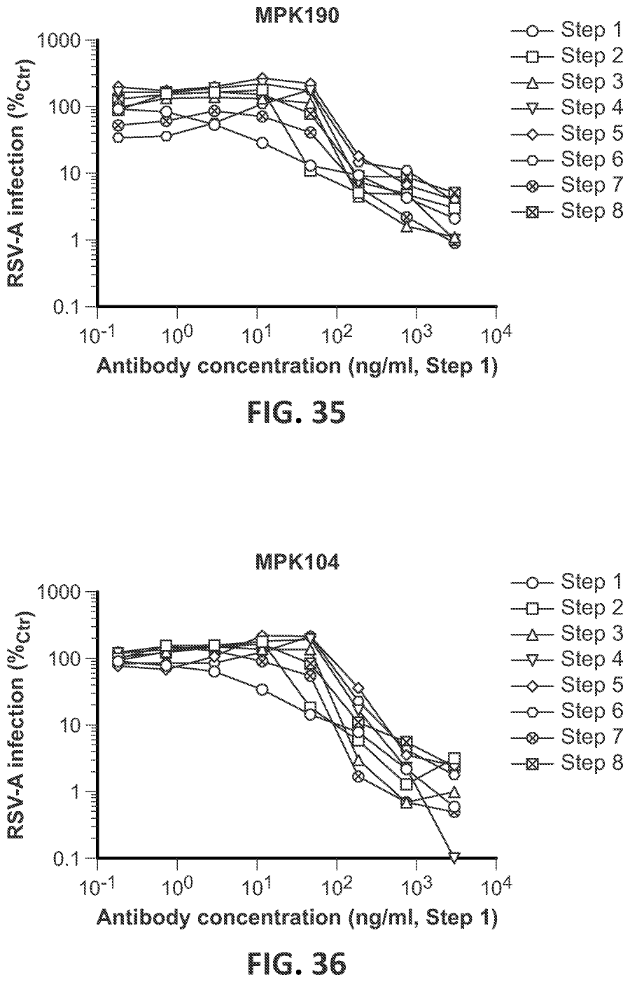

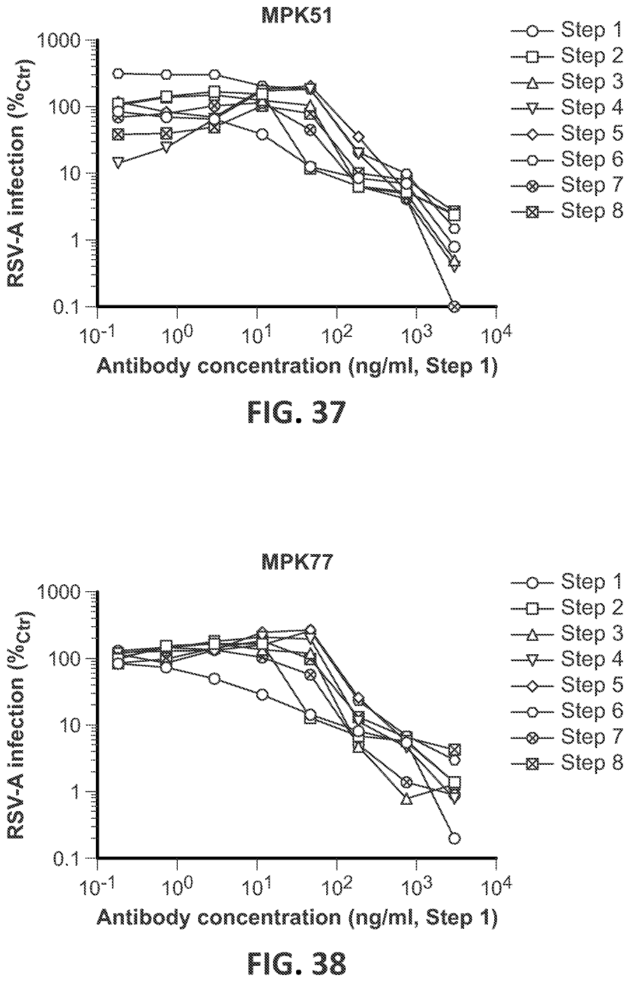

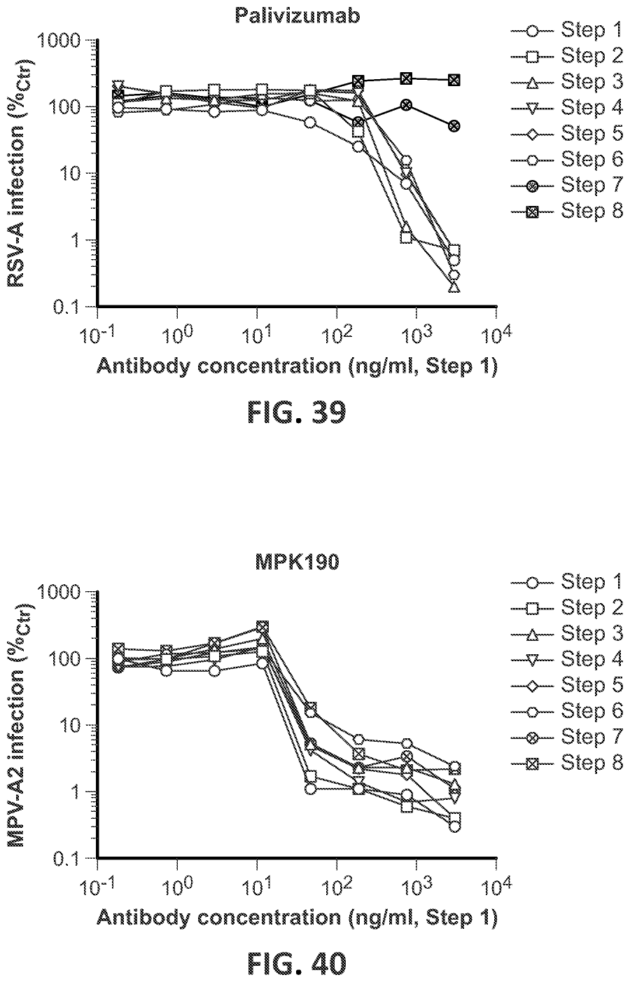

FIGS. 35, 36, 37, 38, and 39, show the results of MPK190, MPK104, MPK51, MPK77, and palivizumab, respectively, incubated with RSV A-GFP added to HEp-2 and LLC-MK2 cells after 8 cycles of reinfection. RSV escape mutants were observed with palivizumab but not with the cross-neutralizing mAbs.

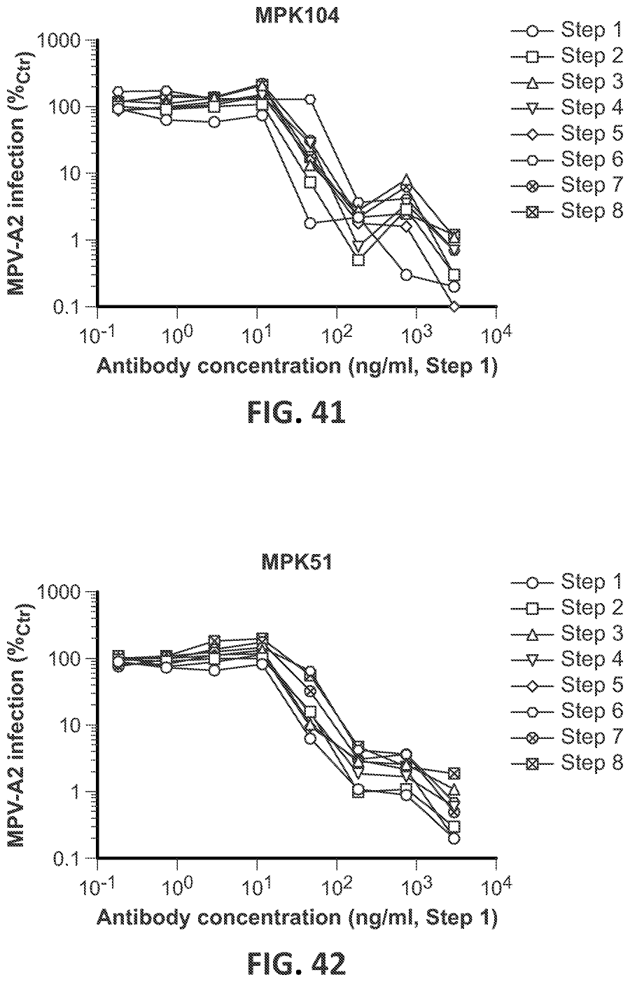

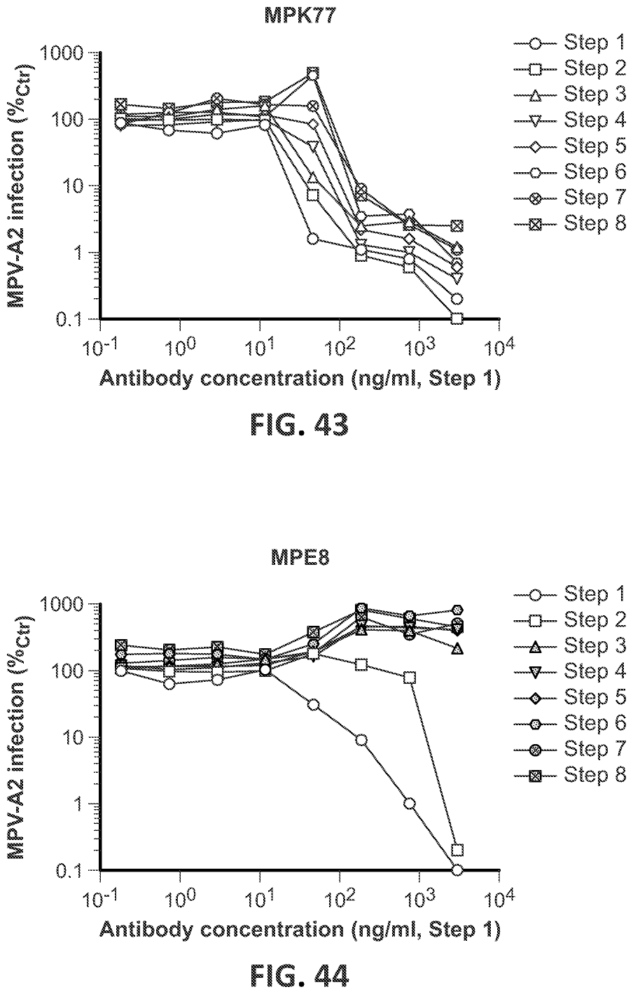

FIGS. 40, 41, 42, 43 and 44, show the results of MPK190, MPK104, MPK51, MPK77, and MPE8, respectively, incubated with MPV A2-GFP added to HEp-2 and LLC-MK2 cells after 8 cycles of reinfection. MPV escape mutants were observed with MPE8, but not with the other cross-neutralizing mAbs. Palivizumab and MPE8 MARMs were confirmed by sequencing. Sequences of RSV and MPV incubated with the candidate mAbs did not show mutations compared to the respective controls.

### Example 22

### Half-Life Extension and In Vivo Pharmacokinetic Analysis of RSV-MPV IgG1-LS mAbs

To extend antibody half-life, mAbs were produced with the LS mutation M428L/N434S in the Fc. LS was chosen in place of other mutations since it maintained effector functions unaltered (YTE is known to reduce effector functions). Additionally, the LA mutation M428L/N434A has been successfully used by Adagio in ADG20 anti-SARS-CoV-2 mAb, which showed an estimated half-life of 96.8 days after a single 300-mg IM dose.

To analyze half-life in vivo, for each pathogen target, different purified antibody IgG variants were tested. Five age- and sex-matched B6.Cg-Fcgrt-tm1Dcr Prkdc-scid Tg(FCGRT)32 Dcr/DcrJ (Scid FcRn−/− hFcRn (32) Tg mice, immune-deficient, hFcRn transgenic, C57B6J background) animals per group were injected with a single bolus of 2-10 mg/kg antibody in sterile physiologic solution via the lateral tail vein. A reference group was injected with a standard recombinant mAb (WT variant without the Fc mutation that extends the half-life). Those mice served as benchmark controls. Animals were used in a range of body weight between 22-30 gr. In each experiment, animals were treated with the same dose and volume of different antibodies. At different time points, ranging from 6 hrs to 40 days post antibody administration (specifically, 2, hrs, 6 hrs, 24 hrs, 3d, 7d, 10d, 14d, 18d, 22d, 26d, 30d, 35d, 40d, 54d, 63d), mice were subjected to a vasodilation step and restrained and bled from lateral tail veins. With a sharp and sterile lancet blade, the lateral vein was nipped to allow some blood extravasation into a capillary tube.

The amount of blood taken each time was adjusted and commensurate to the animal's body weight, in order not to exceed the 20% of the total blood volume withdrawn in two weeks. This means that, for an animal weighing 22 grams, a max volume of 308 1d (assuming a total blood mass of 7% body weight, namely 1.2 mL total volume) was taken in two weeks to allow natural blood pressure compensation. This corresponds to 40 μl each time point. The minimal amount of blood that was feasible to take in order to have the sufficient amount of serum for downstream analyses was 30 μl.

Within 63 days after the injections, animals were terminally euthanized via CO2 asphyxiation to allow recovering blood as well as relevant organs (i.e. spleen, kidney, liver, intestine) for evaluation of preferential antibody accumulation. Experiments were repeated twice for assessing reproducibility and achieving statistical significance.

To prepare the animals for the experiment, upon arrival, animals were housed in individually ventilated cages for one or more week prior to treatment. Before injection and bleeding, animals' cages were warmed up for 10 min under an infrared lamp.

For the intravenous injection of antibodies, animals were initially weighed and then vasodilated by warming up the cage 5 min with an infrared warming lamp. The single mouse was placed in the restrainer to expose the tail. If required, the tail was further warmed in a water bath at 45° c. for 2 min to optimize vasodilation. Before injection, the tail was dried and cleansed with an ethanol pad. Antibody administration was carried out once by injecting in the lateral tail vein 5 ml/kg sterile vehicle (saline or equivalent isotonic buffer) containing 2-10 mg/kg antibody, in a single bolus, with an insulin syringe (27-30-gauge needle). Animals were observed daily for distress signs.

For bleeding, in compliance with the time points described in the experimental plan, animals were first vasodilated for 5 minutes under an infrared lamp, then restrained, tail cleansed with an ethanol pad and bled by puncturing the lateral tail vein by means of sterile, disposable bleeding lancets. Up to 0.05 mL of dripping blood was collected in a capillary placed in a sterile plastic tube. After bleeding, the tail nip was plugged with a sterile gauze for 1 min to allow full hemostasis before returning the animal to the cage.

Animals were observed daily and weighed twice a week. In the event that distress symptoms were observed, the body weight was monitored daily.

A total of six monoclonal antibodies were tested for each of 9 targets. Out of the 6 antibody variants under study, 1 was in a murine isotype and was therefore tested in WT mice (either BALB/c or C57B/6). All the other mAbs were tested in human FcRn transgenic mice (B6.Cg-Fcgrt-tm1Dcr Prkdc-scid Tg(FCGRT)32 Dcr/Dcr). Five mice per group were used and the PK was repeated twice, both in males and females. Antibodies were administered i.v. in a single dose, ranging from 2 to 10 mg/kg. Blood was withdrawn 15 times over 63 days, in a volume of 50 1d or less.

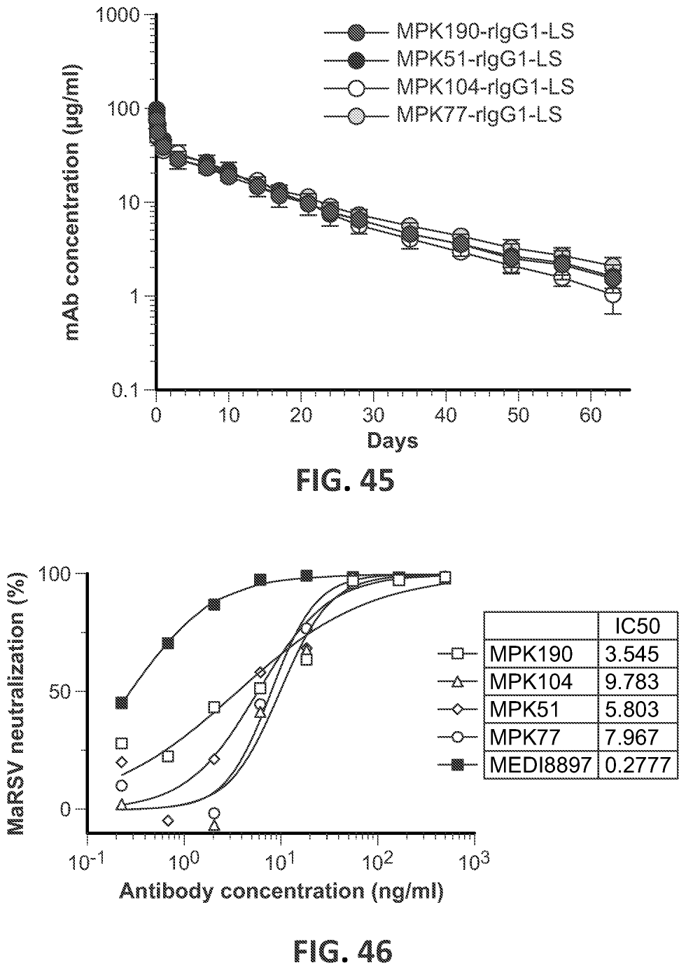

FIG. 45 shows a graph of mAb concentration for MPK190, MPK51, MPK10⁴, and MPK77 versus number of days in the in vivo PK study. The average half-life of IgG1-LS mAbs was 13.36 days±1.09 days. PK results from the study are shown in Table 28.

TABLE 28
Half-life of RSV-MPV mAbs in Tg32 SCID Mice

| | | Half-life | Cavg | CLss | AUClast_D |
|---|---|---|---|---|---|
| Antibody | | (days) | (ug/ml) | (ml/day) | (day*ug/ml/ug) |
| MPK190 | Mean | 14.3 | 52.6 | 1.9 | 6.5 |
| | SD | 1.3 | 10.1 | 0.5 | 0.9 |
| MPK51 | Mean | 13.9 | 64.3 | 1.9 | 5.6 |
| | SD | 1.3 | 9.3 | 0.3 | 1.0 |
| MPK104 | Mean | 12.1 | 56.5 | 1.8 | 6.0 |
| | SD | 1.2 | 7.6 | 0.2 | 0.3 |
| MPK77 | Mean | 15.6 | 48.5 | 2.2 | 6.3 |
| | SD | 1.1 | 3.8 | 0.3 | 0.8 |

### Example 23

### Further Characterization of Selected MPK mAbs

MPK190, MPK104, MPK77 and MPK51 sequences showed very few liabilities. 12 to 16 variants including mutations in either in the HC or the LC were produced for each parental mAb. All the variants were tested and the following features were compared: yield, RSV and MPV neutralization, and binding to RSV F, MPV F and MPV F D280N by FACS. A smaller group of the top variants were purified and tested for polyreactivity, and binding to RSV F, MPV F and MPV F D280N by BLI.

For yield, transient transfection of ExpiCHO cells were used for the production of antibodies. In further detail, the following settings were used:

Materials— Cell culture flasks Erlenmeyer; Mini bioreactor, 50 mL with hydrophobic vented cap; ExpiCHO Expression Medium (Life Technologies, #A2910002); OptiPRO SFM(Life Technologies, #12309-050); ExpiFectamine™ CHO Transfection Kit (Life Technologies, #A29130); Host cell line (see Table 29); Plasmids.

TABLE 29
Host cell line used in production of select antibodies

| | |
|---|---|
| Host cell line | ExpiCHO |
| Growth medium | ExpiCHO Expression Medium |
| Passage | p31 |
| Seeding (day −1) | 3.5 mio cells/ml |
| Concentration at day 0 | 7.4 mio cells/ml |
| Seeding (day 0) | 6.0 mio cells/ml |

Procedure—day-1: Seed cells at 3.5 mio/ml; —Day 0: Cells transfection; Cell count: 7.4 mio/ml; Seed at 6.0 mio vc/ml; Dilute plasmid DNA with cold OptiPRO SFM; DNA: 1 μg for each ml of culture, 0.5 μg Heavy chain and 0.5 μg Light chain; For each 15 ml transfection: add 48 ul of Expifectamine CHO Reagent to the diluted DNA; For each 25 ml transfection: add 80 ul of Expifectamine CHO Reagent to the diluted DNA; Incubate 1 to max 5 minutes and add the mix to the Flask swirling during addition; Incubate cells 8 days at 37° C., shaker incubator 120 rpm; —Day 1: Supplements addition; For each 15 ml transfection: add 3.6 ml of ExpiCHO Feed and 90 ul of ExpiFectamine CHO Enhancer; For each 25 ml transfection: add 6 ml of ExpiCHO Feed and 150 ul of ExpiFectamine CHO Enhancer; Incubate at 37° C., 120 rpm until day 8.

Conditions— Samples dilution: 1:50 in kinetic buffer, both purified mAbs and SNs; Standard curve: Rituximab from 200 μg/ml, 5 vertical dilutions 1:2.5, last dilution at 1.2 ug/ml; QC: Rituximab at known concentration (150, 30, 3.6 ug/ml in KB).

Plates layout—See Table 30 and Table 31; KB: neutralization buffer; RB: regeneration buffer (0.1 M Glycine).

TABLE 30
Plate 1

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | | | |
|---|---|---|---|---|---|---|
| A | 200 | AbD34534- | 317-rIgG1m3- | MPK190- | MPK51- | MPK51- |
| | | mu-IgG2a | LS-G236A | v2.2 | v1.1 | v3.1 |
| B | 80 | Fy1-rIgG1- | FNI9-v5- | MPK190- | MPK51- | MPK51- |
| | | GA | mu-Ig | v2.3 | v1.2 | v3.2 |
| C | 32 | A194-rIgG1- | FM08-mu- | MPK190- | MPK51- | MPK51- |
| | | LS | IgG2a | v3.1 | v1.3 | v3.3 |
| D | 12.8 | A194-rIgG1- | FM08-mu- | MPK190- | MPK51- | MPK51- |
| | | LS-GRLR | IgG2a-N297Q | v3.2 | v1.4 | v3.4 |
| E | 5.12 | A194-rIgG1- | MPK190-v1.1 | MPK190- | MPK51- | MPK51- |
| | | LS-Y300L | | v3.3 | v2.1 | v4.1 |
| F | 2.05 | A194-LS- | MPK190-v1.2 | MPK190- | MPK51- | MPK51- |
| | | G236A-Y300L | | v4.1 | v2.2 | v4.2 |
| G | 1.2 | A194-LS- | MPK190-v1.3 | MPK190- | MPK51- | MPK51- |
| | | G236A-R292P- | | v4.2 | v2.3 | v4.3 |
| | | Y300L | | | | |
| H | KB | 317-rIgG1m3- | MPK190-v2.1 | MPK190- | MPK51- | MPK51- |
| | | LS-EFTAE | | v4.3 | v2.4 | v4.4 |

TABLE 31
Plate 2

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | | | |
|---|---|---|---|---|---|---|
| A | MPK104-v1.1 | MPK104-v3.3 | MPK77-v2.2 | QC 150 | KB | RB |
| B | MPK104-v1.2 | MPK104-v4.1 | MPK77-v2.3 | QC 30 | KB | RB |
| C | MPK104-v1.3 | MPK104-v4.2 | MPK77-v3.1 | QC 3.6 | KB | RB |
| D | MPK104-v2.1 | MPK104-v4.3 | MPK77-v3.2 | KB | KB | RB |
| E | MPK104-v2.2 | MPK77-v1.1 | MPK77-v3.3 | | KB | RB |
| F | MPK104-v2.3 | MPK77-v1.2 | MPK77-v4.1 | | KB | RB |
| G | MPK104-v3.1 | MPK77-v1.3 | MPK77-v4.2 | | KB | RB |
| H | MPK104-v3.2 | MPK77-v2.1 | MPK77-v4.3 | | KB | RB |

Table 32 lists the yield results for the tested antibody variants.

TABLE 32
Quantification of purified antibodies and ExpiCHO SNs

| mAb | Batch | ug/ml | mg/ml |
|---|---|---|---|
| AbD34534-mu-IgG2a | SN | 74.9 | 0.075 |
| FY1-rIgG1-GA | SN | 182.55 | 0.183 |
| A194-rIgG1-LS | SN | 152.55 | 0.153 |
| A194-rIgG1-LS-GRLR | SN | 162.45 | 0.162 |
| A194-rIgG1-LS-Y300L | SN | 195.5 | 0.196 |
| A194-rIgG1-LS-G236A-Y300L | SN | 177.85 | 0.178 |
| A194-rIgG1-LS-G236A-R292P-Y300L | SN | 166.6 | 0.167 |
| 317-rIgG1m3-LS-EFTAE | SN | 174.65 | 0.175 |
| 317-rIgG1m3-LS-G236A | SN | 209.4 | 0.209 |
| FNI9-v5-mu-IgG2a-N297Q | SN | 89.25 | 0.089 |
| FM08-mu-IgG2a (VIR-2482-mu-IgG2a) | SN | 67.3 | 0.067 |
| FM08-mu-IgG2a-N297Q (VIR-2482- | SN | 59.75 | 0.060 |
| mu-IgG2a-N297Q) | | | |
| MPK190-v1.1-rIgG1m17, 1-LS | SN | 292.05 | 0.292 |
| MPK190-v1.2-rIgG1m17, 1-LS | SN | 123 | 0.123 |
| MPK190-v1.3-rIgG1m17, 1-LS | SN | 300.2 | 0.300 |
| MPK190-v2.1-rIgG1m17, 1-LS | SN | 347 | 0.347 |
| MPK190-v2.2-rIgG1m17, 1-LS | SN | 181.8 | 0.182 |
| MPK190-v2.3-rIgG1m17, 1-LS | SN | 299.75 | 0.300 |
| MPK190-v3.1-rIgG1m17, 1-LS | SN | 370.65 | 0.371 |
| MPK190-v3.2-rIgG1m17, 1-LS | SN | 156.45 | 0.156 |
| MPK190-v3.3-rIgG1m17, 1-LS | SN | 308.85 | 0.309 |
| MPK190-v4.1-rIgG1m17, 1-LS | SN | 348.7 | 0.349 |
| MPK190-v4.2-rIgG1m17, 1-LS | SN | 139.95 | 0.140 |
| MPK190-v4.3-rIgG1m17, 1-LS | SN | 272.2 | 0.272 |
| MPK51-v1.1-rIgG1m17, 1-LS | SN | 104.7 | 0.105 |
| MPK51-v1.2-rIgG1m17, 1-LS | SN | 48.5 | 0.049 |
| MPK51-v1.3-rIgG1m17, 1-LS | SN | 61.05 | 0.061 |
| MPK51-v1.4-rIgG1m17, 1-LS | SN | 74.95 | 0.075 |
| MPK51-v2.1-rIgG1m17, 1-LS | SN | 48.2 | 0.048 |
| MPK51-v2.2-rIgG1m17, 1-LS | SN | 0 | 0.000 |
| MPK51-v2.3-rIgG1m17, 1-LS | SN | 46.05 | 0.046 |
| MPK51-v2.4-rIgG1m17, 1-LS | SN | 84.1 | 0.084 |
| MPK51-v3.1-rIgG1m17, 1-LS | SN | 66.15 | 0.066 |
| MPK51-v3.2-rIgG1m17, 1-LS | SN | 48.4 | 0.048 |
| MPK51-v3.3-rIgG1m17, 1-LS | SN | 77.2 | 0.077 |
| MPK51-v3.4-rIgG1m17, 1-LS | SN | 87.05 | 0.087 |
| MPK51-v4.1-rIgG1m17, 1-LS | SN | 71.2 | 0.071 |
| MPK51-v4.2-rIgG1m17, 1-LS | SN | 37.4 | 0.037 |
| MPK51-v4.3-rIgG1m17, 1-LS | SN | 78.6 | 0.079 |
| MPK51-v4.4-rIgG1m17, 1-LS | SN | 120 | 0.120 |
| MPK104-v1.1-rIgG1m17, 1-LS | SN | 177.3 | 0.177 |
| MPK104-v1.2-rIgG1m17, 1-LS | SN | 129.55 | 0.130 |
| MPK104-v1.3-rIgG1m17, 1-LS | SN | 201.6 | 0.202 |
| MPK104-v2.1-rIgG1m17, 1-LS | SN | 170.35 | 0.170 |
| MPK104-v2.2-rIgG1m17, 1-LS | SN | 136.35 | 0.136 |
| MPK104-v2.3-rIgG1m17, 1-LS | SN | 189.25 | 0.189 |
| MPK104-v3.1-rIgG1m17, 1-LS | SN | 190.2 | 0.190 |
| MPK104-v3.2-rIgG1m17, 1-LS | SN | 147.3 | 0.147 |
| MPK104-v3.3-rIgG1m17, 1-LS | SN | 189.85 | 0.190 |
| MPK104-v4.1-rIgG1m17, 1-LS | SN | 186 | 0.186 |
| MPK104-v4.2-rIgG1m17, 1-LS | SN | 197.65 | 0.198 |
| MPK104-v4.3-rIgG1m17, 1-LS | SN | 161.05 | 0.161 |
| MPK77-v1.1-rIgG1m17, 1-LS | SN | 272 | 0.272 |
| MPK77-v1.2-rIgG1m17, 1-LS | SN | 158.95 | 0.159 |
| MPK77-v1.3-rIgG1m17, 1-LS | SN | 160.1 | 0.160 |
| MPK77-v2.1-rIgG1m17, 1-LS | SN | 190.95 | 0.191 |
| MPK77-v2.2-rIgG1m17, 1-LS | SN | 181.9 | 0.182 |
| MPK77-v2.3-rIgG1m17, 1-LS | SN | 233.05 | 0.233 |
| MPK77-v3.1-rIgG1m17, 1-LS | SN | 214.7 | 0.215 |
| MPK77-v3.2-rIgG1m17, 1-LS | SN | 181.75 | 0.182 |
| MPK77-v3.3-rIgG1m17, 1-LS | SN | 168.05 | 0.168 |
| MPK77-v4.1-rIgG1m17, 1-LS | SN | 111.9 | 0.112 |
| MPK77-v4.2-rIgG1m17, 1-LS | SN | 158.5 | 0.159 |
| MPK77-v4.3-rIgG1m17, 1-LS | SN | 154.45 | 0.154 |

For testing binding by FACS, MPK variants MPK104, MPK190, MPK5 1, and MPK77 were tested on MPV F WT and MPV F D280N mutant (Expi293F transfected cells). In further detail, the following settings were used:

Material— Expi293F cells transfected in Exp 16149; MACS Buffer: PBS 1×+2% HyClone+2 mM EDTA; Perm Buffer: Saponin 0.5% in PBS; Fixation: Formaldehyde 4% in PBS (from stock at 37%, Sigma, Cat. No. F1635-500ML); All mAbs have been quantified by Octet at the time of the experiment; mAbs tested in dilution starting 1 μg/mL then 1:4 dilutions. II Ab: 1. goat anti-human IgG AlexaFluor 647 (Jackson ImmunoResearch, Cat. No. 109-606-098) used at 2.5 ug/mL.

Protocol— Counted cells (one sample for all), around 4.5 Mio/mL; Centrifuged cells in minibioreactors, 400 g 5′ RT; Fixation/permeabilization: Resuspended cells to get 10 Mio/mL in FA 4%; Fixed them 20′ at 4° C.; Washed them 1× with MACS Buffer, centrifuged 5′ 400 g RT; Resuspended them at 1 Mio/mL in Perm Buffer, 20 at 4° C.; Washed them 1× with MACS Buffer; Added the mAbs at 0.5 ug/mL or in dilution (in 25 uL Perm Buffer: 25 uL MACS Buffer/well), 50 uL/well, 30′ at 4° C.; Washed the cells 1× with MACS Buffer and added 50 uL/well II Ab (Alexa 647) (in 25 uL Perm Buffer: 25 uL MACS Buffer/well), 30′ at 4° C.; Washed the cells 1× with MACS Buffer and resuspended them in 70 uL/well MACS Buffer; Read them at ZE5 FACS analyzer, 30 uL/well High throughput mode; Read plates 1-8. Stored remaining plates at 4° C. and read them 3 days later.

MPK51, MPK77, MPK104, and MPK190 were tested in neutralization against RSV. The following settings were used:

Materials— Infection medium (IM): MEM (with Earle's salts)+P/S+2.4% HyClone; Detection solution (DS): Draq5 5× (1:300) in IM; MPK mAbs tested and internal controls (MPE8-Vir8000, RSD5, MPH12, MEDI8897, D25, RSB27, palivizumab, MPE33 and MPF5); All mAbs have been quantified by Octet; Included also previously purified parental mAbs (MPK51, MPK77, MPK104 and MPK190) as reference control.

Protocol— 28 Apr. 2022: Diluted mAbs in IM (2′000 ng/ml (final 500 ng/ml)→1:3 serial dilutions 8×) and transferred 10 uL in each well of 384 well plates (Integra); Used same mAbs dilution for MPV neutralization (Exp 16181) performed the same day; Added 10 uL/well virus in IM (containing 300 TCID50/well); Incubated the mix 45′ at 37° C.; Added HEp-2 cells in 20 uL/well in IM (1000 cells/well); Let virus grow for 6 days; —4 May 2022: Added 10 ul/well detection solution and incubated at least 3 h at 37° C.; Read the plates at Mirrorball with 10 μm resolution and gated for GFP positive cells (% of total).

Table 33 shows IC50 values from the neutralization tests against RSV. Antibody variants that do not neutralize were set at 2000 ng/ml. MPK51-v2.2 does not have any value since it was used in dilution.

TABLE 33
IC50 values (ng/ml) of MPK antibody variants
and controls in neutralization against RSV

| mAb | IC50 (ng/mL) | mAb | IC50 (ng/mL) |
|---|---|---|---|
| MPK190-v1.1 | 5.801 | MPK104-v3.1 | 4.929 |
| MPK190-v1.2 | 7.151 | MPK104-v3.2 | 8.762 |
| MPK190-v1.3 | 8.937 | MPK104-v3.3 | 6.088 |
| MPK190-v2.1 | 2.118 | MPK104-v4.1 | 6.363 |
| MPK190-v2.2 | 3.766 | MPK104-v4.2 | 3.823 |
| MPK190-v2.3 | 4.741 | MPK104-v4.3 | 3.52 |
| MPK190-v3.1 | 5.704 | MPK77-v1.1 | 6.14 |
| MPK190-v3.2 | 6.147 | MPK77-v1.2 | 4.888 |
| MPK190-v3.3 | 3.128 | MPK77-v1.3 | 4.509 |
| MPK190-v4.1 | 4.207 | MPK77-v2.1 | 5.125 |
| MPK190-v4.2 | 2.468 | MPK77-v2.2 | 15.89 |
| MPK190-v4.3 | 1.979 | MPK77-v2.3 | 4.437 |
| MPK51-v1.1 | 6.637 | MPK77-v3.1 | 3.707 |
| MPK51-v1.2 | 14.37 | MPK77-v3.2 | 2.907 |
| MPK51-v1.3 | 7.53 | MPK77-v3.3 | 5.946 |
| MPK51-v1.4 | 12.15 | MPK77-v4. I | 1.34 |
| MPK51-v2 1 | 16.07 | MPK77-v4.2 | 4.546 |
| MPK51-v2.2 | | MPK77-v4.3 | 5.048 |
| MPK51-v2 3 | 13.82 | MPK51-rl Gl/ZV3 | 5.06 |
| MPK51-v2.4 | 11.48 | MPK51-rIgG1/YY1 | 5.267 |
| MPK51-v3.1 | 7.8 | MPK77-rIgG1/WH2 | 7.482 |
| MPK51-v3.2 | 20.69 | MPK104-rIgGl/AA2 | 6.984 |
| MPK51-v3.3 | 11.59 | MPK19CrIgG1/KYT | 10.52 |
| MPK51-v3 4 | 8.734 | MPK190-rIgGl/4B6 | 6.995 |
| MPK51-v4.1 | 7.765 | RSB27-rIgG1-LS | 0.5108 |
| MPK51-v4.2 | 25.81 | MPE33-rIgG1 | 2000 |
| MPK51-v4 3 | 11.4 | MPF5 VH117D-rIgGl | 2000 |
| | | (V2) | |
| MPK51-v4.4 | 9.858 | MPH12-rIgG1-LS | 12.92 |
| MPK104-v1.1 | 6.606 | MPE8-Vir8000-rIgGI- | 12.15 |
| | | LS | |
| MPK104-v1.2 | 5.23 | D25-rIgGl | 0.929 |
| MPK104-v1.3 | 4.796 | RSD5-FR-GL-rIgG1 | 1.198 |
| MPK104-v2.1 | 7.268 | MED18897-rIgG1- | 0.5486 |
| | | YTE | |
| MPK104-v2.2 | 5.921 | palivizumab-rIgG1 | 55.23 |
| MPK104-v2.3 | 5.708 | | |

MPK51, MPK77, MPK104, and MPK190 were tested in neutralization against MPV. The following settings were used:

Materials— Infection medium (IM): MEM (with Earle's salts)+P/S+2.4% HyClone; Detection solution (DS): Draq5 5× (1:300) in IM; MPK mAbs tested and internal controls (MPE8-Vir8000, RSD5, MPH12, MED18897, D25, RSB27, palivizumab, MPE33 and MPF5); All mAbs have been quantified by Octet; Included also previously purified parental mAbs (MPK5 1, MPK77, MPK104 and MPK190) as reference control.

Protocol— 28 Apr. 2022: Diluted mAbs in IM (2000 ng/ml (final 500 ng/ml)→1:3 serial dilutions 8×) and transferred 10 uL in each well of 384 well plates (Integra); Used the same dilutions for Exp 16180 (RSV Neutralization) performed the same day; Added 10 uL/well virus in IM (containing 300 TCID50/well); Incubated the mix 45′ at 37° C.; Added LLC-MK2 cells in 10 uL/well in IM (1000 cells/well); Let virus grow for 6 days; —29 Apr. 2022: Added 20 uL/well TPCK-Trypsin (final concentration 45 μg/mL, initial concentration 112.5 ug/mL); —4 May 2022: Added 10 ul/well detection solution and incubated at least 3 h at 37° C.; Read the plates at Mirrorball with 10 μm resolution and gated for GFP positive cells (% of total).

Table 34 shows IC50 values from the neutralization tests against MPV. Antibody variants that do not neutralize were set at 2000 ng/ml. MPK51-v2.2 does not have any value since it was used in dilution, and it does not neutralize MPV.

TABLE 34
IC50 values (ng/ml) of MPK antibody variants
and controls in neutralization against MPV

| | IC50 | | IC50 |
|---|---|---|---|
| mAb | (ng/mL) | mAb | (ng/mL) |
| MPK190-v1.1 | 0.7046 | MPK104-v3.1 | 3.025 |
| MPK190-v1.2 | 2.375 | MPK104-v3.2 | 3.342 |
| MPK190-v1.3 | 1.474 | MPK104-v3.3 | 5.994 |
| MPK190-v2.1 | 1.565 | MPK104-v4.1 | 1.438 |
| MPK190-v2.2 | 2.422 | MPK104-v4.2 | 1.258 |
| MPK190-v2.3 | 2.531 | MPK104-v4.3 | 1.164 |
| MPK190-v3.1 | 1.511 | MPK77-v1.1 | 2.095 |
| MPK190-v3.2 | 2.272 | MPK77-v1.2 | 4.396 |
| MPK190-v3.3 | 2.071 | MPK77-v1.3 | 4.86 |
| MPK190-v4.1 | 1.502 | MPK77-v2.1 | 99.08 |
| MPK190-v4.2 | 2.591 | MPK77-v2.2 | 939.1 |
| MPK190-v4.3 | 1.659 | MPK77-v2.3 | 1033 |
| MPK51-v1.1 | 5.876 | MPK77-v3.1 | 4.002 |
| MPK51-v1.2 | 9.122 | MPK77-v3.2 | 552 |
| MPK51-v1.3 | 4.216 | MPK77-v3.3 | 33.17 |
| MPK51-v1.4 | 5.104 | MPK77-v4.1 | 2.706 |
| MPK51-v2.1 | 8.821 | MPK77-v4.2 | 536.2 |
| MPK51-v2.2 | | MPK77-v4.3 | 2000 |
| MPK51-v2.3 | 35.93 | MPK51-rIgG1/ZV3 | 2.849 |
| MPK51-v2 4 | 547.7 | MPK51-rIgG1/YY1 | 2.306 |
| MPK51-v3.1 | 6.096 | MPK77-rIgG1/WH2 | 3.848 |
| MPK51-v3.2 | 713.6 | MPK104-rIgG1/AA2 | 1.885 |
| MPK51-v3 3 | 4.095 | MPK190-rIgG1/KYT | 1.941 |
| MPK51-v3 4 | 538.3 | MPK190-rIgG1/4B6 | 2.004 |
| MPK51-v4.1 | 5.41 | RSB27-rIgG1-LS | 2000 |
| MPK51-v4.2 | 319.5 | MPE33-rIgG1 | 14.97 |
| MPK51-v4.3 | 4.721 | MPF5 VH117D-rIgG1 (V2) | 5.63 |
| MPK51-v4.4 | 292.2 | MPH12-rIgG1-LS | 5.258 |
| MPK104-v1.1 | 1.359 | MPE8-Vir8000-rIgG1-LS | 10.99 |
| MPK104-v1.2 | 2.494 | D25-rIgG1 | 2000 |
| MPK104-v1.3 | 1.158 | RSD5-FR-GL-rIgG1 | 2000 |
| MPK104-v2.1 | 1.808 | MED18897-rIgG1-YTE | 2000 |
| MPK104-v2.2 | 1.323 | palivizumab-rIgG1 | 2000 |
| MPK104-v2.3 | 1.123 | | |

Polyreactivity of selected MPK antibody variants, including deamidated were tested. The following settings were used:

Conditions— Polyreactivity tested on HEp-20-10 (Euroimmun 1522-2010); mAb concentration: 50 ug/ml; mAb controls: Positive (polyreactive): F16-rIgG1-v3.11.18-CHO, Negative (non polyreactive): MPE8-rIgG1-v3; Incubation times: 1h; Microscopy conditions (Axio Vert.A1+ZEN Software); Exposure time: 980 ms; Analog gain: 2×.

Protocol— Dilute mAbs in PBS-Tween (1 ml Tween in 500 ml PBS) at the desired concentration; Put a 25 ul drop on the apposite hydrophobic support; Put the slide upside down on the drops; Incubate 1h at RT; 1× rapid wash+1× wash 5 min shaking in PBS-Tween; Put a 20 ul drop of Alexa488 anti hu IgG Fc gamma specific 1.5 mg/ml (109-545-098) at 3 μg/ml in PBS-Tween on the hydrophobic support (this secondary reagent is less prone to bleaching than the one provided with the kit); Remove excess washing solution and put the slide upside down on the drops; Incubate 1h at RT; 1× rapid wash+1× wash 5 min shaking in PBS-Tween; Remove excess washing solution, add ca 10 ul glycerol/biochip and mount coverslip; Take pictures at the Axio Vert.A1.

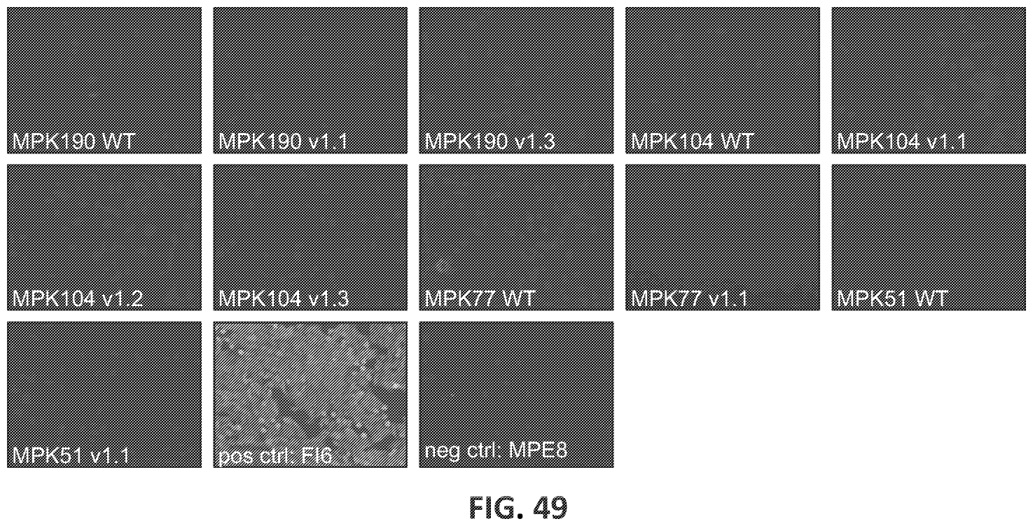

MPK104, MPK190, MPK51, and MPK77 antibody variants did not exhibit polyreactivity (FIG. 49).

For binding tests, MPK190, MPK51, MPK104 and MPK77 were tested for binding to RSV-F, MPV-F and MPV-F-D280N. The following settings were used:

Material— Kinetics Buffer (KB) 1× no tween (BSA 0.01% in PBS, pH 7.1).

Conditions— SA-sensors; 96 well plate black →32 sensor mode; pH 7.1, 1000 rpm, 30° C.; volume: 200 ul/well; Sensor Hydratation: >10 min in KB; Loading of F-proteins: in KB, 10 min, concentrations are: 0.306 ug/ml for biot-RSV-F wt, 0.357 ug/ml for biot-MPV-F wt, 0.425 ug/ml for biot-MPV-F D280N (reference); Association of the mAb: starting 5 μg/ml in KB, 4 min; Dissociation of mAb: 6 min in KB; Baselines: in KB, 60 sec.

Procedure—Pre-hydrate sensors in KB (see conditions) for at least 10 min; In the meanwhile prepare plate; Run experiment as follows:
RUN1: baseline in A1 (p 1)→Loading in A5 (p 1)→baseline in A5 (p2)→Association in A9 (p2)→dissociation in A5 (p2)
RUN2: baseline in A1 (p 1)→Loading in A9 (p 1)→baseline in A5 (p2)→Association in A9 (p2)→dissociation in A5 (p2)
RUN3: baseline in A1 (p1)→Loading in(A1 (p2)→baseline in A5 (p2)→Association in A9 (p2)→dissociation in A5 (p2)

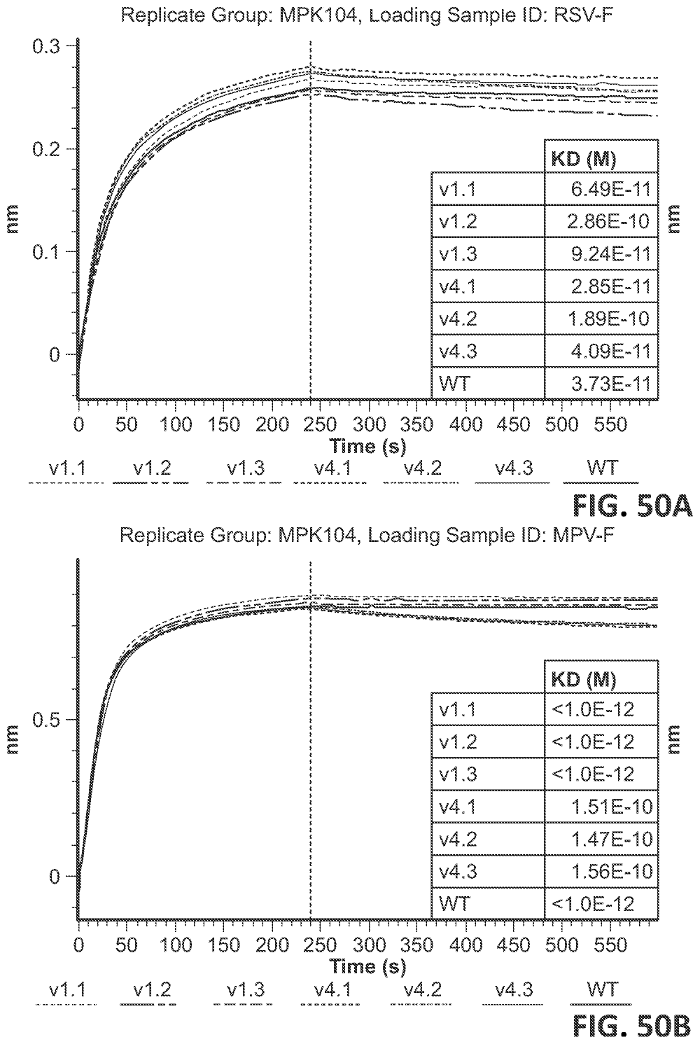

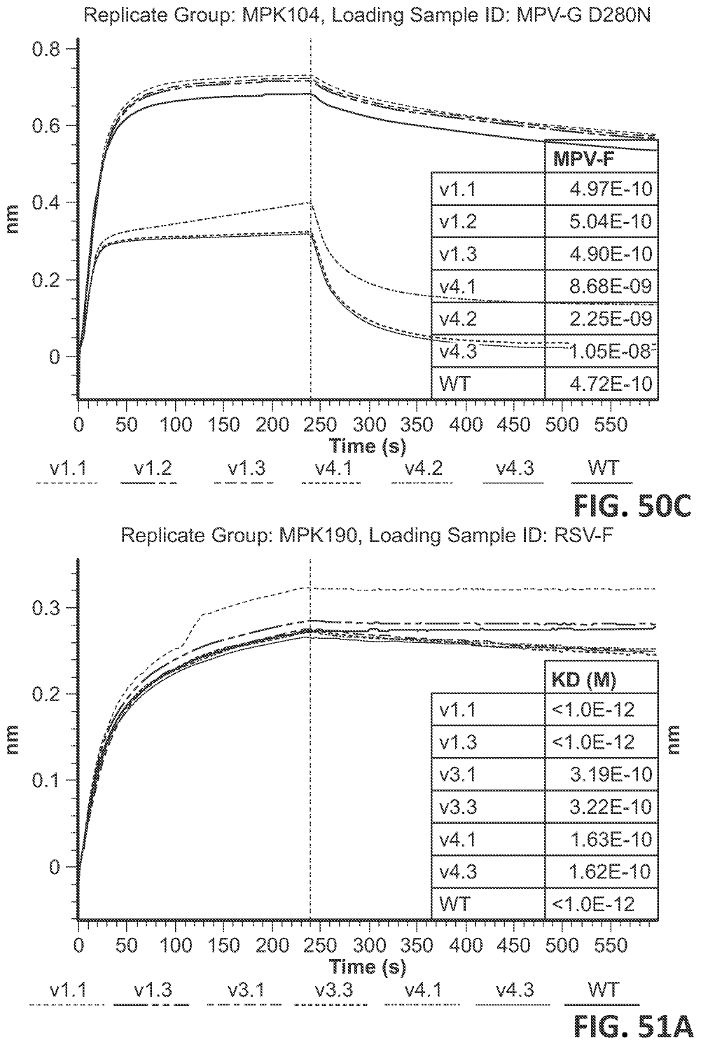

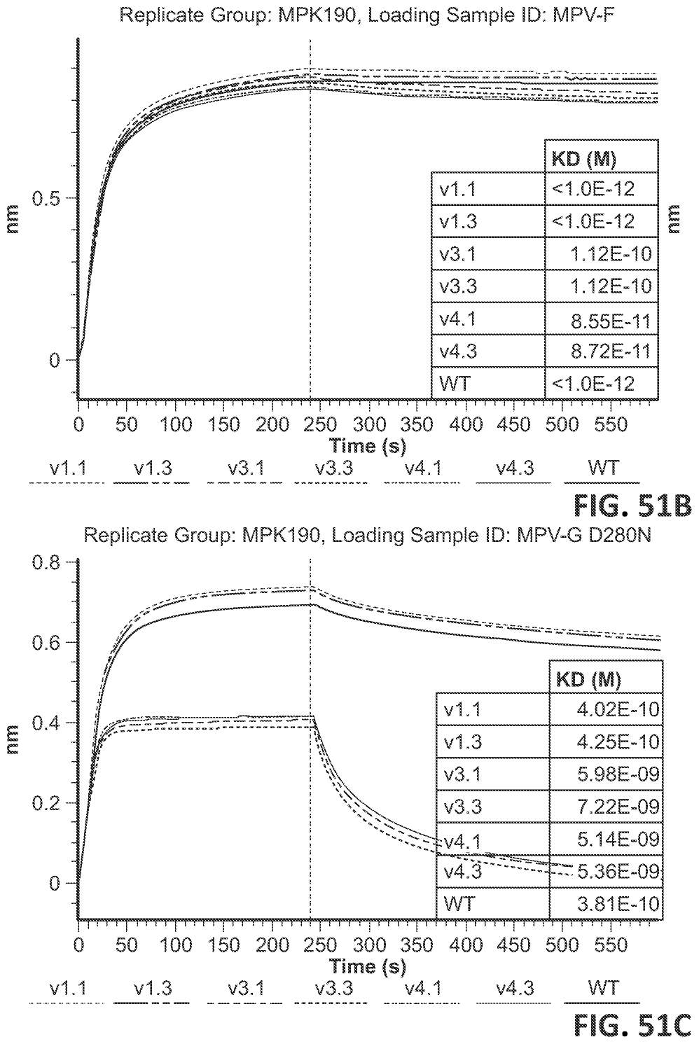

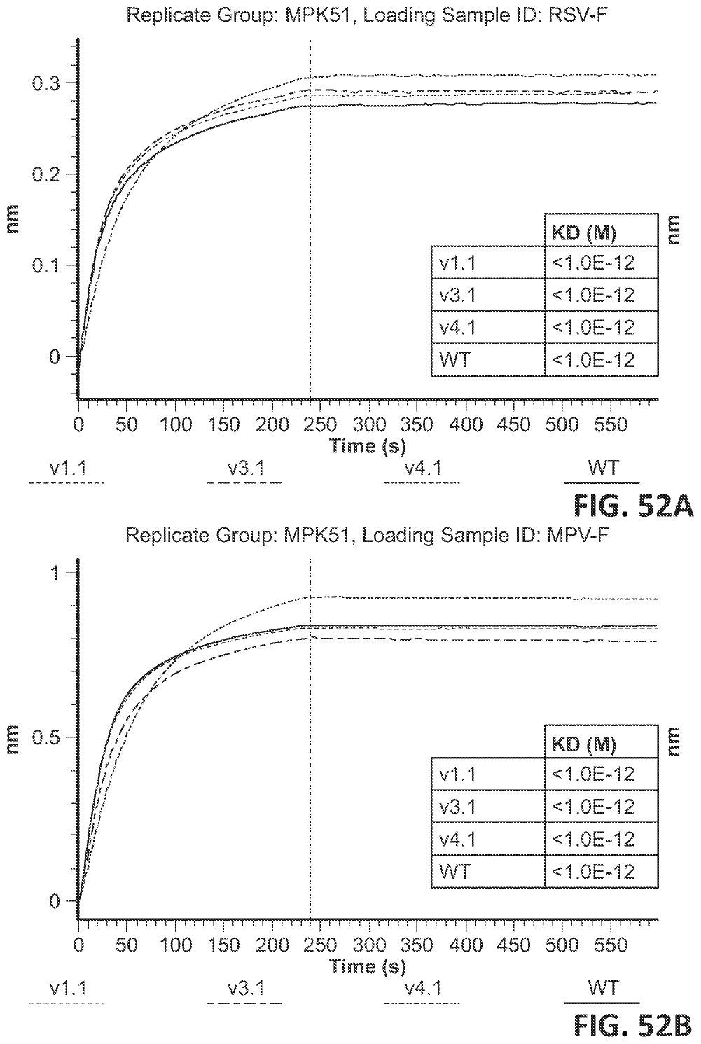

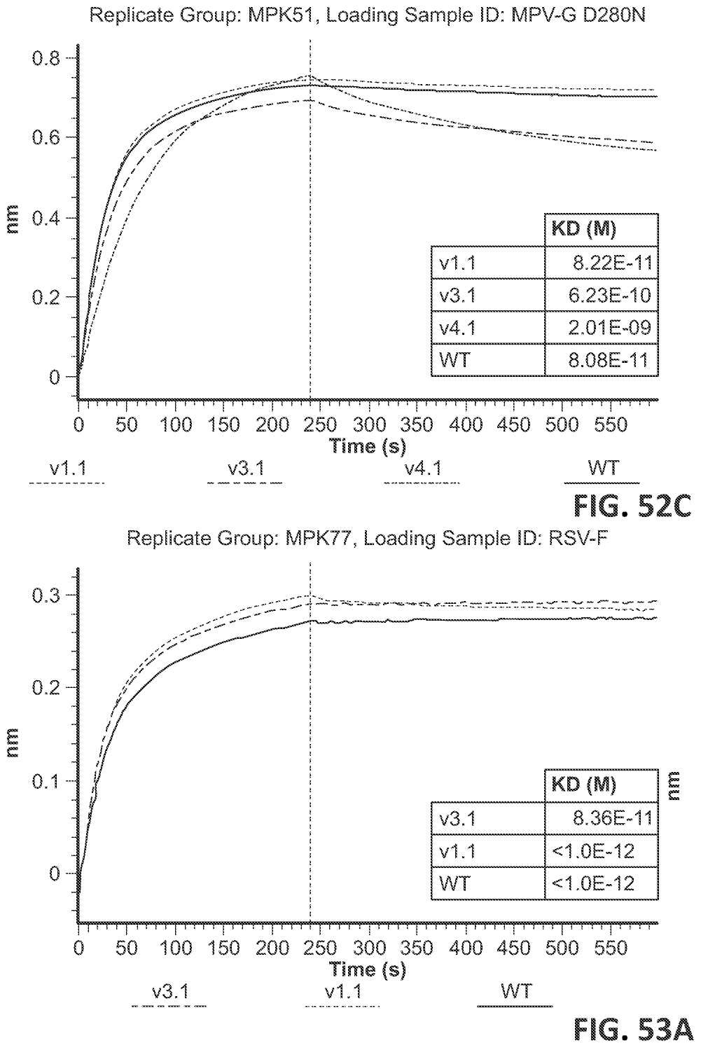

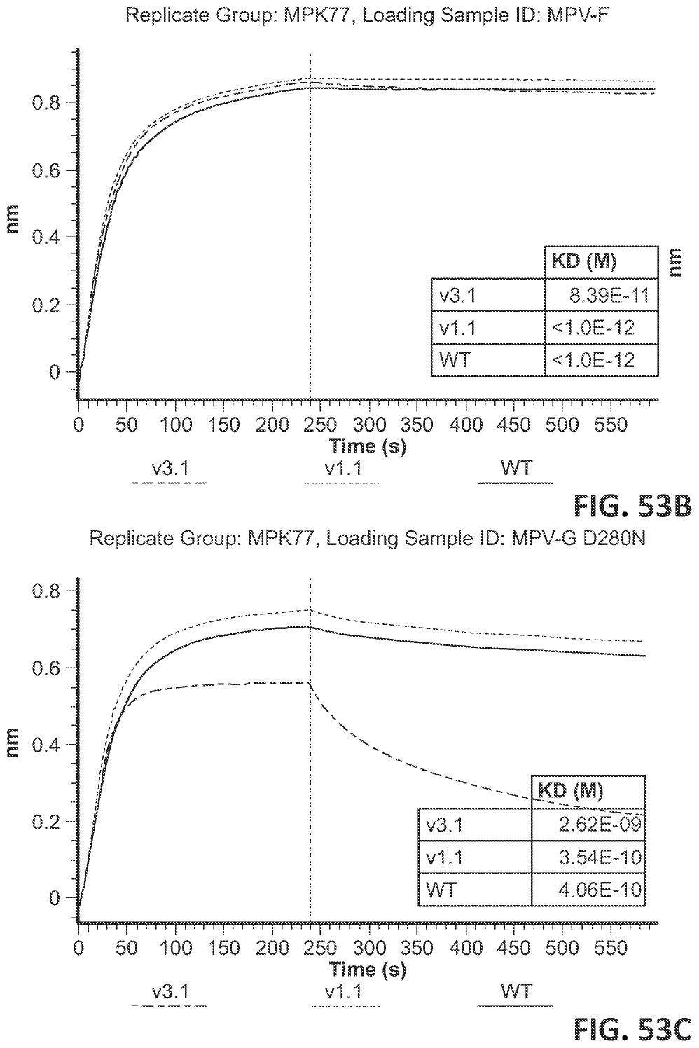

MPK104, MPK190, MPK51, and MPK77 antibody variants binding to RSV-F (FIGS. 50A, 51A, 52A, 53A), MPV-F (FIGS. 50B, 51B, 52B, 53B), and MPV-F D280N (FIGS. 50C, 51C, 51B, 51C) was assessed by BLI.

Table 35 shows a comparison of MPK190 variants to parental mAb MPK190-v1.1. MPK190-v1.3 maintained yield, RSV and MPV neutralization, binding to RSV-F, MPV F and MPV D280N F at levels similar to the parental mAb (v.1).

TABLE 35
Comparison of MPK190 variants to parental mAb MPK190-v1.1

| | | Binding (FACS) | | | | |
|---|---|---|---|---|---|---|
| | | | MPV F | MPV F | Neutralization | |
| mAb | Yield | RSV F | WT | D280N | RSV A | MPV A2 |
| MPK190-v1.1 | 1.0 | 1.0 | 1.0 | 1.0 | 1.0 | 1.0 |
| MPK190-v1.2 | 0.0 | 0.8 | 0.9 | 0.8 | 1.0 | 1.0 |
| MPK190-v1.3 | 1.1 | 0.9 | 0.9 | 0.9 | 1.0 | 1.0 |
| MPK190-v2.1 | 1.5 | 0.8 | 0.8 | 0.0 | 1.0 | 1.0 |
| MPK190-v2.2 | 0.0 | 0.8 | 0.8 | 0.0 | 1.0 | 1.0 |
| MPK190-v2.3 | 1.1 | 0.9 | 0.8 | 0.0 | 1.0 | 1.0 |
| MPK190-v3.1 | 1.8 | 0.9 | 0.9 | 0.6 | 1.0 | 1.0 |
| MPK190-v3.2 | 0.0 | 0.7 | 0.8 | 0.3 | 1.0 | 1.0 |
| MPK190-v3.3 | 1.2 | 0.8 | 0.9 | 0.5 | 1.0 | 1.0 |
| MPK190-v4.1 | 1.6 | 0.9 | 0.9 | 0.6 | 1.0 | 1.0 |
| MPK190-v4.2 | 0.0 | 0.7 | 0.8 | 0.4 | 1.0 | 1.0 |
| MPK190-v4.3 | 0.8 | 0.9 | 0.9 | 0.5 | 1.0 | 1.0 |

Table 36 shows a comparison of MPK51 variants to parental mAb MPK51-v1.1. All the MPK51 variants showed loss of yield, binding and neutralization compared to the parental mAb (v1.1).

TABLE 36
Comparison of MPK190 variants to parental mAb MPK190-v1.1

| | | Binding (FACS) | | | | |
|---|---|---|---|---|---|---|
| | | | MPV F | MPV F | Neutralization | |
| mAb | Yield | RSV F | WT | D280N | RSV A | MPV A2 |
| MPK51-v1.1 | 1.0 | 1.0 | 1.0 | 1.0 | 1.0 | 1.0 |
| MPK51-v1.2 | 0.0 | 0.6 | 0.5 | 0.0 | 0.5 | 1.0 |
| MPK51-v1.3 | 0.0 | 0.8 | 0.8 | 0.3 | 1.0 | 1.0 |
| MPK51-v1.4 | 0.7 | 0.9 | 0.6 | 0.0 | 0.5 | 1.0 |
| MPK51-v2.1 | 0.0 | 0.6 | 0.6 | 0.0 | 0.5 | 1.0 |
| MPK51-v2.2 | 0.0 | 0.9 | 0.1 | 0.0 | 0.0 | 0.0 |
| MPK51-v2.3 | 0.0 | 0.6 | 0.3 | 0.0 | 0.5 | 0.0 |
| MPK51-v2.4 | 0.8 | 0.8 | 0.0 | 0.0 | 0.5 | 0.0 |
| MPK51-v3.1 | 0.0 | 0.7 | 0.8 | 0.7 | 1.0 | 1.0 |
| MPK51-v3.2 | 0.0 | 0.5 | 0.0 | 0.0 | 0.0 | 0.0 |
| MPK51-v3.3 | 0.7 | 0.8 | 0.7 | 0.0 | 0.5 | 1.0 |
| MPK51-v3.4 | 0.8 | 0.8 | 0.0 | 0.0 | 1.0 | 0.0 |
| MPK51-v4.1 | 0.0 | 0.8 | 0.8 | 0.6 | 1.0 | 1.0 |
| MPK51-v4.2 | 0.0 | 0.4 | 0.2 | 0.0 | 0.0 | 0.0 |
| MPK51-v4.3 | 0.7 | 0.9 | 0.7 | 0.0 | 0.5 | 1.0 |
| MPK51-v4.4 | 1.2 | 0.9 | 0.4 | 0.0 | 1.0 | 0.0 |

Table 37 shows a comparison of MPK14 variants to parental mAb MPK104-v1.1. MPK104-v2.3 maintained yield, RSV and MPV neutralization, binding to RSV-F, MPV F and MPV D280N F at levels similar to the parental mAb (v1.1).

TABLE 37
Comparison of MPK104 variants to parental mAb MPK104-v1.1

| | | Binding (FACS) | | | | |
|---|---|---|---|---|---|---|
| | | | MPV F | MPV F | Neutralization | |
| mAb | Yield | RSV F | WT | D280N | RSV A | MPV A2 |
| MPK104-v1.1 | 1.0 | 1.0 | 1.0 | 1.0 | 1.0 | 1.0 |
| MPK104-v1.2 | 0.5 | 0.9 | 1.0 | 1.0 | 1.0 | 1.0 |
| MPK104-v1.3 | 1.2 | 1.1 | 1.0 | 1.1 | 1.0 | 1.0 |
| MPK104-v2.1 | 0.9 | 1.1 | 0.8 | 0.0 | 1.0 | 1.0 |
| MPK104-v2.2 | 0.6 | 1.1 | 0.8 | 0.0 | 1.0 | 1.0 |
| MPK104-v2.3 | 1.1 | 1.2 | 0.8 | 0.0 | 1.0 | 1.0 |
| MPK104-v3.1 | 1.1 | 0.9 | 0.9 | 0.1 | 1.0 | 1.0 |
| MPK104-v3.2 | 0.7 | 0.8 | 0.9 | 0.1 | 1.0 | 1.0 |
| MPK104-v3.3 | 1.1 | 1.1 | 1.0 | 0.2 | 1.0 | 1.0 |
| MPK104-v4.1 | 1.1 | 1.3 | 1.0 | 0.4 | 1.0 | 1.0 |
| MPK104-v4.2 | 1.2 | 1.2 | 0.9 | 0.4 | 1.0 | 1.0 |
| MPK104-v4.3 | 0.8 | 1.2 | 0.9 | 0.4 | 1.0 | 1.0 |

Table 38 shows a comparison of MPK77 variants to parental mAb MPK77-v1.1. All the MPK77 variants showed loss of yield, binding and neutralization compared to the parental mAb (v1.1).

TABLE 38
Comparison of MPK77 variants to parental mAb MPK77-v1.1

| | | Binding (FACS) | | | | |
|---|---|---|---|---|---|---|
| | | | MPV F | MPV F | Neutralization | |
| mAb | Yield | RSV F | WT | D280N | RSV A | MPV A2 |
| MPK77-v1.1 | 1.0 | 1.0 | 1.0 | 1.0 | 1.0 | 1.0 |
| MPK77-v1.2 | 0.0 | 0.7 | 0.8 | 0.0 | 1.0 | 1.0 |
| MPK77-v1.3 | 0.0 | 0.8 | 0.8 | 0.1 | 1.0 | 1.0 |
| MPK77-v2.1 | 0.2 | 1.1 | 0.7 | 0.2 | 1.0 | 0.0 |
| MPK77-v2.2 | 0.0 | 0.3 | 0.0 | 0.0 | 0.5 | 0.0 |
| MPK77-v2.3 | 0.6 | 0.7 | 0.0 | 0.0 | 1.0 | 0.0 |
| MPK77-v3.1 | 0.4 | 0.8 | 0.9 | 0.6 | 1.0 | 1.0 |
| MPK77-v3.2 | 0.0 | 0.6 | 0.6 | 0.0 | 1.0 | 0.0 |
| MPK77-v3.3 | 0.0 | 0.7 | 0.6 | 0.0 | 1.0 | 0.0 |
| MPK77-v4.1 | 0.0 | 1.0 | 0.7 | 0.1 | 1.0 | 1.0 |
| MPK77-v4.2 | 0.0 | 0.3 | 0.0 | 0.0 | 1.0 | 0.0 |
| MPK77-v4.3 | 0.0 | 0.6 | 0.0 | 0.0 | 1.0 | 0.0 |

Among all the variants tested, only two variants MPK104-v-1.3 and MPK190-v1.3 maintained yield, RSV and MPV neutralization, binding and affinity for RSV-F, MPV F and MPV-F D280N at levels similar to their parental mAbs.

### Example 24

### Forced Deamidation of MPK190 Variants

The NG deamidation motif in H-FR3 of MPK190 and MPK104 is the only potential liability. NG74 in heavy chain FR3 potentially contacts the antigen. The NG motif cannot be removed by N74Q substitution (loss of affinity for D280N). The NG motif is known to be subject to deamidation under harsh stress conditions (forced deamidation at pH10).

A forced deamidation test was performed on MPK190-rIgG1-LS, using the following settings:

Materials— 100 mM Tris, pH 8.0; Slide-A-Lyzer™ G2 Dialysis Cassettes, 10K MWCO, 3 mL.

Sample—MPK190-rIgG1-LS 20211202/4B6 3.208 mg/ml 3.8 mg (1.18 ml).

Procedure—1 Jun. 2022: Diluted sample to 2 mg/ml in Tris buffer; Added sample to Slide-A-Lyzer Cassette following producer protocol; Dialysis: Incubated 1h in Tris buffer at 4° C., gently stirring, Changed the buffer and incubated 1h at 4° C., Changed the buffer and incubated overnight at 4° C.; —2 Jun. 2022: Checked sample concentration in Nanodrop: 1.39 mg/ml; Prepared 3 aliquots of 1.1 mg each; Snap-freezed 1 aliquot and store at −80° C.; Stored the 2 remnant aliquots at 40° C.; —9 Jun. 2022: Snap-freezed 1 aliquot and store at −80° C.→Day 7; —14 Jun. 2022: Snap-freezed 1 aliquot and store at −80° C.→Day 12.

Tables 39 and 40 show motifs in the heavy chain and light chain of MPK190 and MPK104.

TABLE 39
Antibody motifs in heavy chain of MPK190 and MPK104

| Name | Residues | Position (from, to) |
|---|---|---|
| Oxidation (W/M) in CDRH1 | M | (34, 34) |
| Oxidation (W/M) in CDRH2 | M | (64, 64) |
| N-terminal E | E | (1, 1) |
| Asp isomeration | DS | (62, 63) |
| (DS\|DD\|DN\|DY\|DR\|DP) in CDRH2 | | |
| Asn deamidation | N | (74) |

TABLE 40
Antibody motifs in light chain of MPK190 and MPK104

| | | Position |
|---|---|---|
| Name | Residues | (from, to) |
| Asp isomeratoin | DR | (94, 95) |
| (DS\|DD\|DN\|DY\|DR\|DP) in CDRL3 | | |
| Asn deamidation | NS | (26, 27) |
| (NS\|NN\|NH\|NT\|NY\|NQ\|NF\|NG) in CDRL1 | | |
| Asn deamidation | NN | (53, 54) |
| (NS\|NN\|NH\|NT\|NY\|NQ\|NF\|NG) in CDRL2 | | |

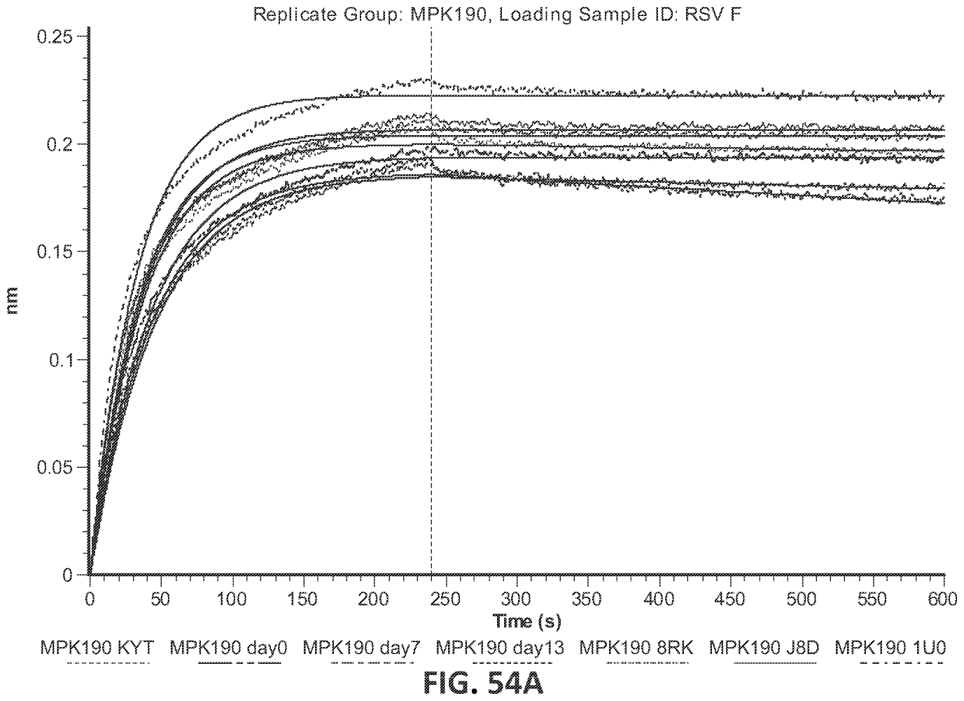

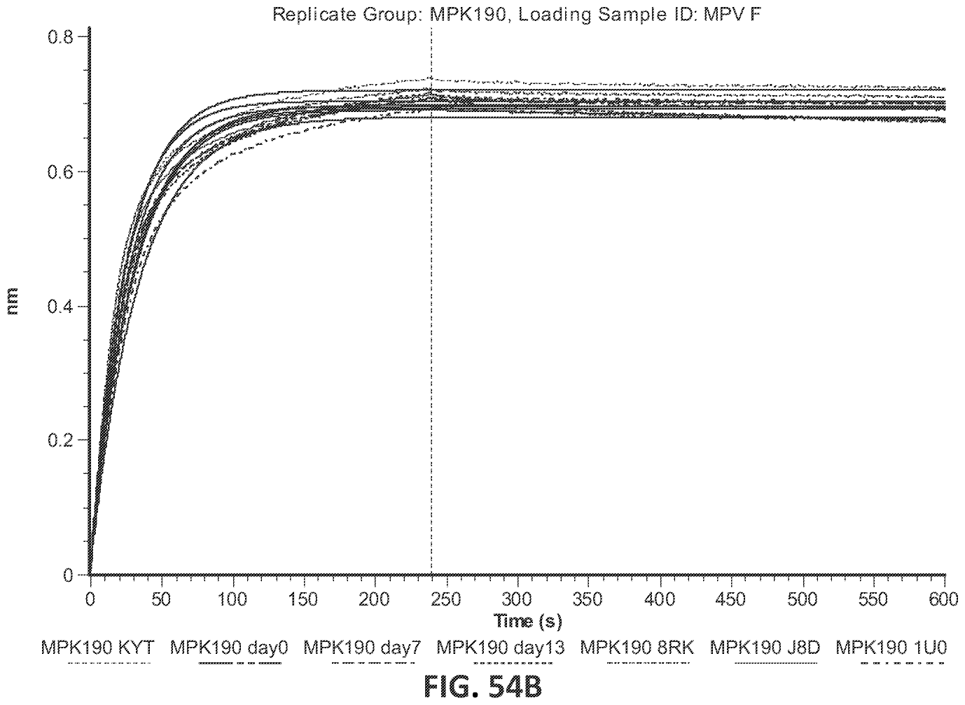

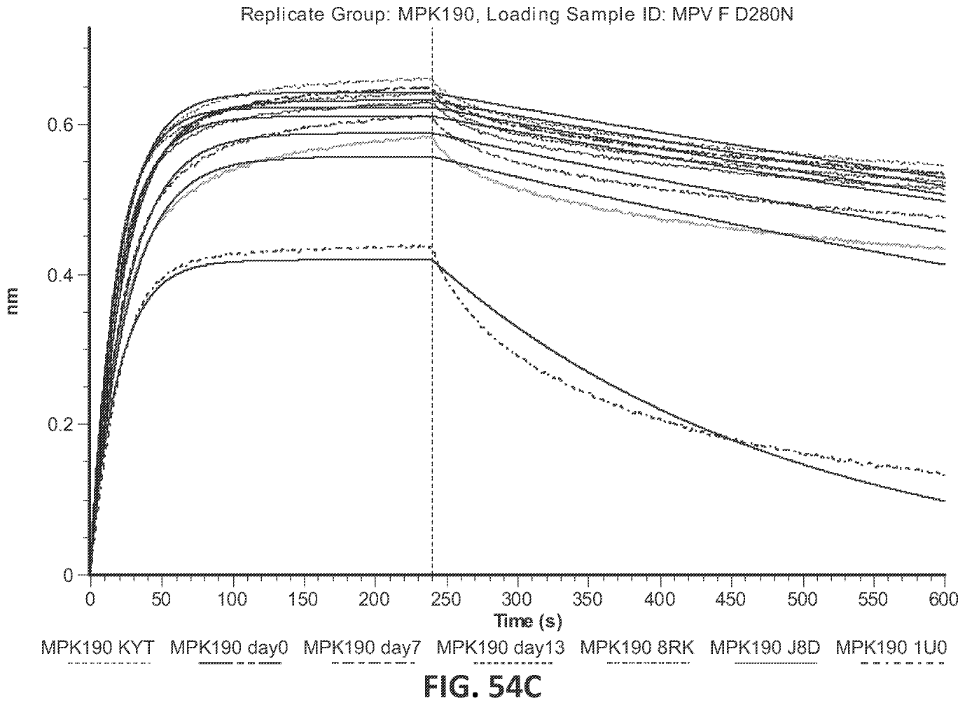

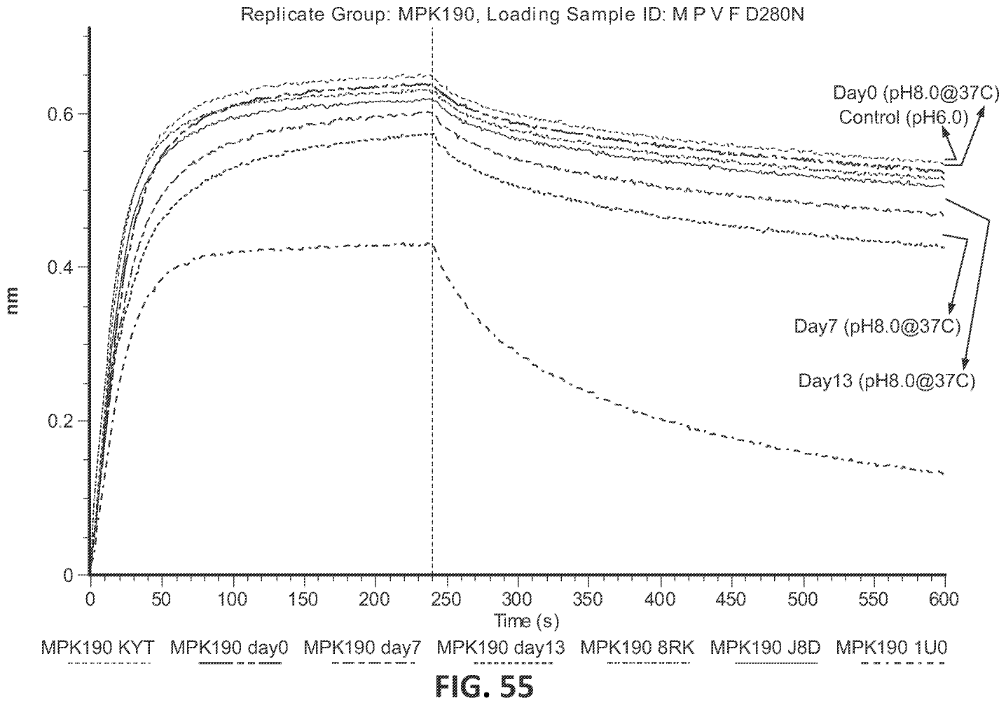

Binding of MPK190 after forced deamidation to RSV-F (FIG. 54A), MPV-F (FIG. 54B), and MPV-F D280N (FIG. 54C) was assessed by BLI. The affinity of a MPK190 variant with an NG motif had minor loss after forced deamidation (FIG. 55). Affinity of an additional MPK190 variant (v5.3) with an NA motif was reduced for the MPV-F D280N mutant. The results were also confirmed by flow cytometry using cells expressing F proteins.

Table 41 shows potency of MPK190 variants after forced deamidation against RSV-A, RSV-B, and MPV. All variants displayed similar potency against RSV-a, RSV-B, and MPV. Forced deamidation did not appear to affect potency. Potency against MPV-D280N was not able to be assessed.

TABLE 41
IC50 of MPK190 variants after forced deamidation
against RSV-A, RSV-B, and MPV

| | IC50 (ng/mL) | | |
|---|---|---|---|
| | RSV A GFP | RSV B GFP | MPV A GFP |
| MPK190-rIgG1-LS | 3.881 | 3.363 | 7.898 |
| MPK190-rIgG1-LS day 0 | 3.209 | 4.098 | 7.217 |
| MPK190-rIgG1-LS day 7 | 2.94 | 4.401 | 5.803 |
| MPK190-rIgG1-LS day 13 | 7.024 | 4.508 | 8.394 |
| MPK190-v1.1-rIgG1-LS | 3.54 | 3.394 | 4.03 |
| MPK190-v1.30rIgG1-LS | 3.184 | 2.383 | 5.992 |
| MPK-v5.3-rIg-G1-LS (NA) | 4.336 | 2.995 | 7.017 |

### Example 25

### Neutralization Of Stressed Material

MPK190 stressed antibody variants were tested in neutralization against RSV A and RSV B. MPK190 day 0, 7 and 12 of stress at 40° C. as well as NA version (v5.3) were tested. Also tested were MPK77 and MPK10⁴ selected variants. The following settings were used:

Material— Infection medium (IM): MEM (with Earle's salts)+P/S+2.4% HyClone; Detection solution (DS): Draq5 5× (1:300) in IM; MPK mAbs tested and internal controls (MPE8-Vir8000, RSD5, MPH12, MED18897); All but MPK190 stressed variants mAbs and MPK190-v5.3 have been quantified by Octet (Nanodrop for these 4 mAbs); Control mAbs (MEDI8897, RSD5, MPE8 and MPH12 are on every plate).

Protocol— 13 Jun. 2022: Diluted mAbs in IM (2′000 ng/ml (final 500 ng/ml)→1:3 serial dilutions 8×) and transferred 10 uL in each well of 384 well plates (Integra); Used same mAbs dilution for MPV neutralization (Exp 16537) performed the same day; Added 10 uL/well virus in IM (containing 300 TCID50/well); Incubated the mix 45′ at 37° C.; Added HEp-2 cells in 20 uL/well in IM (1000 cells/well); Let virus grow for 6 days; —17 Jun. 2022 (lecture after 3 days, first plates): Added 10 ul/well detection solution and incubated at least 3 h at 37° C.; Read the plates at Mirrorball with 10 μm resolution and gated for GFP positive cells (% of total); — 20 Jun. 2022 (lecture after 6 days, second plates): Added 10 ul/well detection solution and incubated at least 3 h at 37° C.; Read the plates at Mirrorball with 10 μm resolution and gated for GFP positive cells (% of total).

RSV A GFP and RSV B GFP results after 6 days with IC50 values are shown in Table 42.

TABLE 42
RSV Neutralization by MPK190 stressed
antibody variants - IC50 (ng/ml)

| | RSV A GFP | RSV B GFP |
|---|---|---|
| MPK190-rIgG1-LS/KYT | 3.881 | 3.363 |
| MPK190-rIgG1-LS day O | 3.209 | 4.098 |
| MPK190-rIgG1-LS day 7 | 2.94 | 4.401 |
| MPK190-rIgG1-LS day 12 | 7.024 | 4.508 |
| MPK190-v1.1-rIgG1-LS | 3.54 | 3.394 |
| MPK190-v1.3-rIgG1-LS | 3.184 | 2.383 |
| MPK190-v5.3-rIgG1-LS NA | 4.336 | 2.995 |
| MPK77-rIgG1-LS | 6.771 | 7.319 |
| MPK77-v1.1-rIgG1-LS | 5.066 | 4.465 |
| MPK104-rIgG1-LS | 7.44 | 5.233 |
| MPK104-v1.3-rIgG1-LS | 2.709 | 6.491 |
| MEDI8897-rIgG1-YTE | 0.4827 | 0.4193 |
| RSD5-FR-GL-rIgG1 | 1.275 | 1.144 |
| MPE8-Vir8000-rIgG1-LS | 4.411 | 3.55 |
| MPH12-rIgG1-LS | 6.549 | 5.233 |
| MP K190-rlgG1-LS/4B6 | 3.727 | 6.391 |

MPK190 stressed antibody variants were also tested in neutralization against MPV. MPK190 day 0, 7 and 12 of stress at 40° C. as well as NA version (v5.3) were tested. Also tested were MPK77 and MPK10⁴ selected variants. The following settings were used:

Materials— Infection medium (IM): MEM (with Earle's salts)+P/S+2.4% HyClone; Detection solution (DS): Draq5 5× (1:300) in IM; MPK mAbs tested and internal controls (MPE8-Vir8000, RSD5, MPH12, MEDI8897); All but MPK190 stressed variants mAbs and MPK190-v5.3 have been quantified by Octet (Nanodrop for these 4 mAbs); Control mAbs (MEDI8897, RSD5, MPE8 and MPH12 are on every plate).

Protocol— 13 Jun. 2022: Diluted mAbs in IM (2′000 ng/ml (final 500 ng/ml)→1:3 serial dilutions 8×) and transferred 10 uL in each well of 384 well plates (Integra); Used the same dilutions for Exp 16536 (RSV Neutralization) performed the same day; Added 10 uL/well virus in IM (containing 300 TCID50/well); Incubated the mix 45′ at 37° C.; Added LLC-MK2 cells in 10 uL/well in IM (1000 cells/well); Let virus grow for 6 days; —14 Jun. 2022: Added 20 15 uL/well TPCK-Trypsin (final concentration 45 μg/mL, initial concentration 112.5 ug/mL) only for the plates that are going to be read later (20 Jun. 2022); — 17 Jun. 2022 (first plates): Added 10 ul/well detection solution and incubated at least 3 h at 37° C.; Read the plates at Mirrorball with 10 μm resolution and gated for GFP positive cells (% of total); — 20 Jun. 2022 (second plates): Added 10 ul/well detection solution and incubated at least 3 h at 37° C.; Read the plates at Mirrorball with 10 μm resolution and gated for GFP positive cells (% of total).

MPV A GFP results after 6 days with IC50 values are shown in Table 43.

TABLE 43
MPV A GFP Neutralization by MPK190 stressed
antibody variants - IC50 (ng/ml)

| | MPV A GFP |
|---|---|
| MPK190-rIgG1-LS/KYT | 7.898 |
| MPK190-rIgG1-LS day O | 7.217 |
| MPK190-rIgG1-LS day 7 | 5.803 |
| MPK190-rIgG1-LS day 13 | 8.394 |
| MPK190-v1.1-rIgG1-LS | 4.03 |
| MPK190-v1.3-rIgG1-LS | 5.992 |
| MPK190-v5.3-rIgG1-LS NA | 7.017 |
| MPK77-rIgG1-LS | 12.92 |
| MPK77-v1.1-rIgG1-LS | 6.799 |
| MPK104-rIgG1-LS | 6.794 |
| MPK104-v1.3-rIgG1-LS | 5.568 |
| MED18897-rIgG1-YTE | 2000 |
| RSD5-FR-GL-rIgG1 | 2000 |
| MPE8-Vir8000-rIgG1-LS | 24.71 |
| MPH12-rIgG1-LS | 13 |
| MP K190-rlgG1-LS/4B6 | 6.158 |

### Example 26

### Polyreactivity of Selected MPK Antibody Variants

Tests were run to determine if selected MPK antibody variants are polyreactive. The following settings were used:

Materials— HEp20-10 slides, EurImmune, Cat. No. 1522-2010; Positive control mAb: F16-rIgG1-v3 11.18-CHO (20180410/8CG, 6.608 mg/mL) 50 μg/mL; Dilution of samples, II Ab and washing steps: PBS-Tween20 0.2% (1 mL Tween in 500 mL PBS); II Ab: goat anti-human IgG Fc gamma specific Alexa 488 (Jackson Immuno Research, Cat. No. 109-545-098, 1.5 mg/mL) 3 μg/mL; Incubation times: 1h; Microscopy conditions (Axio Vert.A1+ZEN Software): Exposure time: 980 ms, Analog gain: 2×.

Samples—all at 50 μg/mL
1. Negative control (non polyreactive mAb): MPE8-rIgG1-v3
2. Positive control (polyreactive mAb): F16 rIgG1-v3 11.18-CHO

Protocol— 14 Jun. 2022: Diluted MPK mAbs at 50 μg/mL in PBS-Tween. Dilute positive and negative control mAbs at 50 μg/mL; Added 25 μL of diluted sample on the squares of the hydrophobic support; Flipped the slide with HEp cells to get them in contact with the samples; Incubation time 1h at room temperature; After 1h, quickly rinsed with PBS-Tween the slide with HEp cells then washed the slide 5′ RT in PBS-Tween on a rocking platform. In the meantime, carefully rinse the hydrophobic support to remove samples (use dH2O); Added 20 uL II Ab on the squares of the hydrophobic support. Removed excess PBS-Tween from the slide and flipped it to get HEp cells in contact with II Ab; Incubation time 1h at room temperature; After 1h, quickly rinsed with PBS-Tween the slide with HEp cells then washed the slide 5′ RT in PBS-Tween on a rocking platform. In the meantime, carefully rinse the hydrophobic support to remove II Ab (use dH₂O); Removed excess PBS-Tween from the slide and added 10 uL glycerol on a coverslip (coverslip on the hydrophobic support to know where to add glycerol=on the squares); Flipped the slide with HEp cells to mount it with coverslip; Looked at the cells with Axio Vert Fluorescence microscope, ZEN Software; Exposure time 980 ms, analog gain 2×.

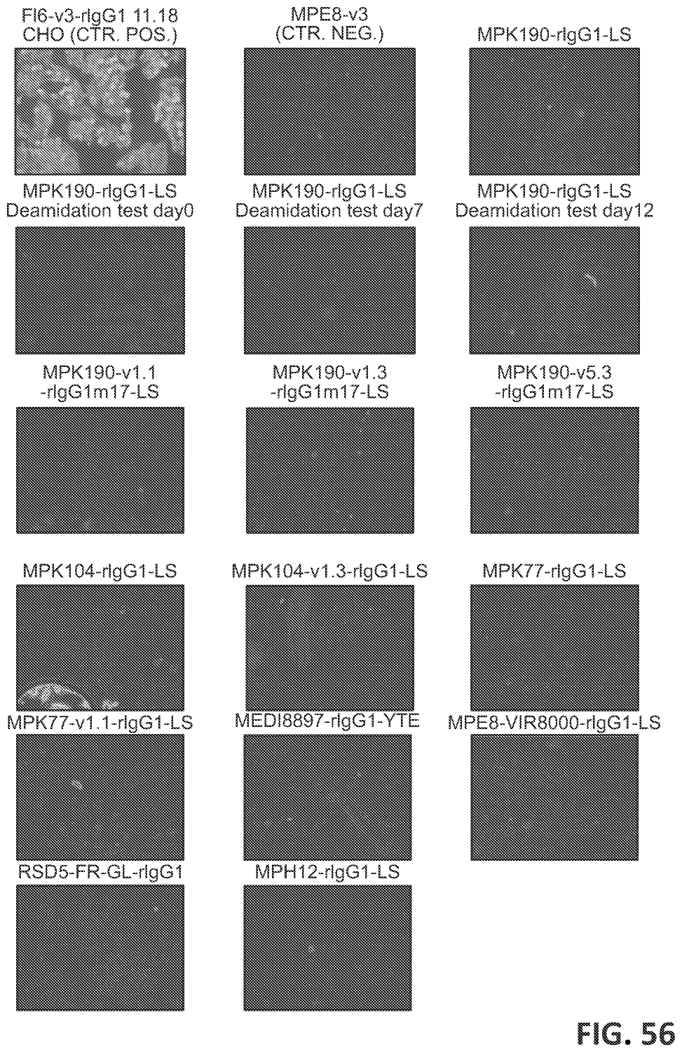

No polyreactivity was observed (FIG. 56).

### Example 27

### Binding (Octet) Tests on MPK190 and Other Selected MPK Variants for Binding to RSV F, MPV F, and MPV F D280N, and Mouse Sera Testing

Binding was assessed with the following settings:

Material— Kinetics Buffer (KB) lx no tween (BSA 0.01% in PBS, pH 7.1).

Conditions— SA-sensors; 96 well plate black →16 sensor mode; pH 7.1, 1000 rpm, 30° C.; volume: 200 ul/well; Sensor Hydratation: >10 min in KB; Loading of F-proteins: in KB, 10 min, concentrations are: 0.306 ug/ml for biot-RSV-F wt, 0.357 ug/ml for biot-MPV-F wt, 0.425 ug/ml for biot-MPV-F D280N; mAbs association: 5 μg/ml in KB, 4′; mAbs dissociation: 6′ in KB; Baselines: in KB, 60″.

Procedure—Pre-hydrate sensors in KB (see conditions) for at least 10 min; In the meanwhile prepare plate as follows in Table 44.

TABLE 44
Plate preparation for binding tests

| | 1 | 2 | 3 | 4 | 5 | 6 | 7 | 8 | 9 | 10 | 11 | 12 |
|---|---|---|---|---|---|---|---|---|---|---|---|---|
| A | RSV F | RSV F | MPV F | MPV F | MPV F | MPVF | MPK190-rIgG1-LS | MPK104-v1.3-rIgG1-LS | KB | KB | KB | KB |
| B | | | | | D280N | D280N | MPK190-rIgG1-LS day 0 | MPK77-rIgG1-LS | | | | |
| C | | | | | | | MPK190-rIgG1-LS day 7 | MPK77-v1.1-rIgG1-LS | | | | |
| D | | | | | | | MPK190-rIgG1-LS day 12 | MEDI8897-rIgG1-YTE | | | | |
| E | | | | | | | MPK190-v1.1-rIgG1-LS | RSD5-FR-GL-rIgG1 | | | | |
| F | | | | | | | MPK190-v1.3-rIgG1-LS | MPE8-Vir8000-rIgG1-LS | | | | |
| G | | | | | | | MPK190-v5.3-rIgG1-LS NA | MPH12-rIgG1-LS | | | | |
| H | | | | | | | MPK104-rIgG1-LS | KB | | | | |

Run experiment as follows:
RUN 1: baseline in 9-10→Loading in 1-2→baseline in 9-10→Association in 7-8→dissociation in 9-10
RUN 2: baseline in 9-10→Loading in 3-4→baseline in 9-10→Association in 7-8→dissociation in 9-10
RUN 3: baseline in 9-10→Loading in 5-6→baseline in 9-10→Association in 7-8→dissociation in 9-10

Binding results are shown in Table 45.

TABLE 45
Results of bindings tests on MPK190 and other selected MPK
variants for binding to RSV F, MPV F, and MPV F D280N

| | | KD (M) | KD (M) | KD (M) |
|---|---|---|---|---|
| mAb | Batch | RSV F | MPV F | MPV F D280N |
| MPK190-rlgG1-LS | 20220105/KYT | 5.39611 | <1.0E−12 | 3.70E−10 |
| MPK190-rlgG1-LS day 0 | 20220602/RU8 | <1.0E−12 | <1.0E−12 | 4.41E−10 |
| MPK190-rlgG1-LS day 7 | 20220602/RU8 | 1.28E−10 | 1.59E−11 | 6.00E−I0 |
| MPK190-rlgG1-LS day 13 | 20220602/RU8 | 3.04E−10 | 5.77611 | 7.33E−10 |
| MPK190-v1.1-rlgG1-LS | 20220511/8RK | <1.0E−12 | <1.0E−12 | 3.62E−10 |
| MPK190-v1.3-rlgG1-LS | 20220511/J8D | <1.0E−12 | <1.0E−12 | 3.95E−10 |
| MPK190-v5.3-rIgG1-LS NA | 20220614/1U0 | <1.0E−12 | 4.93612 | 2.91E−09 |
| MPK104-rlgG1-LS | 20210430/AA8 | 3.06E−10 | <1.0E−12 | 3.91E−10 |
| MPK104-v1.3-rlgG1-LS | 20220512/G2S | 2.09E−10 | <1.0E−12 | 3.70E−10 |
| MPK77-rlgG1-LS | 20210602/WH2 | 2.28E−12 | <1.0E−12 | 3.94E−10 |
| MPK77-v1.1-rlgG1-LS | 20220512/DFO | <1.0E−12 | <1.0E−12 | 2.98E−10 |
| MED18897-rlgG1-YTE | 20180928/ZAB | 3.97E−11 | No binding | No binding |
| RSD5-FR-GL-rlgG1 | 20171219/BJU | 1.46E−09 | No binding | No binding |
| MPE8-Vir8000-rlgG1-LS | 20180903/WXL | <1.0E−12 | 2.87609 | No binding |
| MPH12-rlgGI-LS | 20191122/WBM | 1.20E−09 | <1.0E−12 | 1.01E−09 |

### Example 28

### Binding (Facs) Tests on MPK190 and Other Selected MPK Variants for Binding to RSV F, MPV F, AND MPV F D280N (Exp1239F Transfected Cells)

Material— Exp1293F cells transfected in Exp 16557; MACS Buffer: PBS 1×+2% HyClone+2 mM EDTA; Perm Buffer: Saponin 0.5% in PBS; Fixation: Formaldehyde 4% in PBS (from stock at 37%, Sigma, Cat. No. F1635-500ML); Table 46 shows the antibody variants tested in dilution starting 1 μg/ml then 0.5 ug/ml then 1:3 dilutions; II Ab: goat anti-human IgG AlexaFluor 647 (Jackson ImmunoResearch, Cat. No. 109-606-098) used at 2.5 ug/mL.

TABLE 46
mAbs tested in dilution

| mAb | ug/ml | mAb | ug/ml |
|---|---|---|---|
| MPK190-rlgG1-LS | 1940 | MPK104-v1.3-rIgG1-LS | 2877 |
| MPK190-rIgG1 day 0 | 1002.15 | MPK77-rIgG1-LS | 3616 |
| MPK190-rIgG1 day 7 | 1001.4 | MPK77-v1.1-rIgG1-LS | 6532 |
| MPK190-rIgG1 day 13 | 1004.2 | MEDI8897-rIgG1-YTE | 2 |
| MPK190-v1.1-rIgG1-LS | 1162 | RSD5-FR-GL-rIgG1 | 4133 |
| MPK190-v1.3-rIgG1-LS | 1048 | MPE8-Vir8000-rIgG1-LS | 4774 |
| MPK190-v5.3-rIgG1-LS | 0.811 | MPH12-rIgG1-LS | 3490 |
| NA | | | |
| MPK104-rIgG1-LS | 2349 | S2X2S9-v5-rIgG1-LS | 5629 |

Protocol— Counted cells (one sample for all: RSV F), around 4.9 Mio/mL; Add 25 mL PBS and centrifuged cells in minibioreactors, 400 g 5′ RT; Fixation/permeabilization; Resuspended cells to get 10 Mio/mL in FA 4%; Fixed them 20′ at 4° C.; Added MACS Buffer up to get 1 Mio cells/mL and plated 150 uL/well in 96 well plates U bottom; Spin 5′ 400 g RT; Added 150 uL/well Perm Buffer, 20 at 4° C.; Spin 5′ 400 g RT; Added diluted mAbs (in 25 uL Perm Buffer: 25 uL MACS Buffer/well), 50 uL/well, 30′ at 4° C.; Washed the cells 1× with MACS Buffer and added 50 uL/well II Ab (Alexa 647) (in 25 uL Perm Buffer: 25 uL MACS Buffer/well), 30′ at 4° C.; Washed the cells 1× with MACS Buffer and resuspended them in 70 uL/well MACS Buffer; Read them at ZE5 FACS analyzer, 30 uL/well High throughput mode.

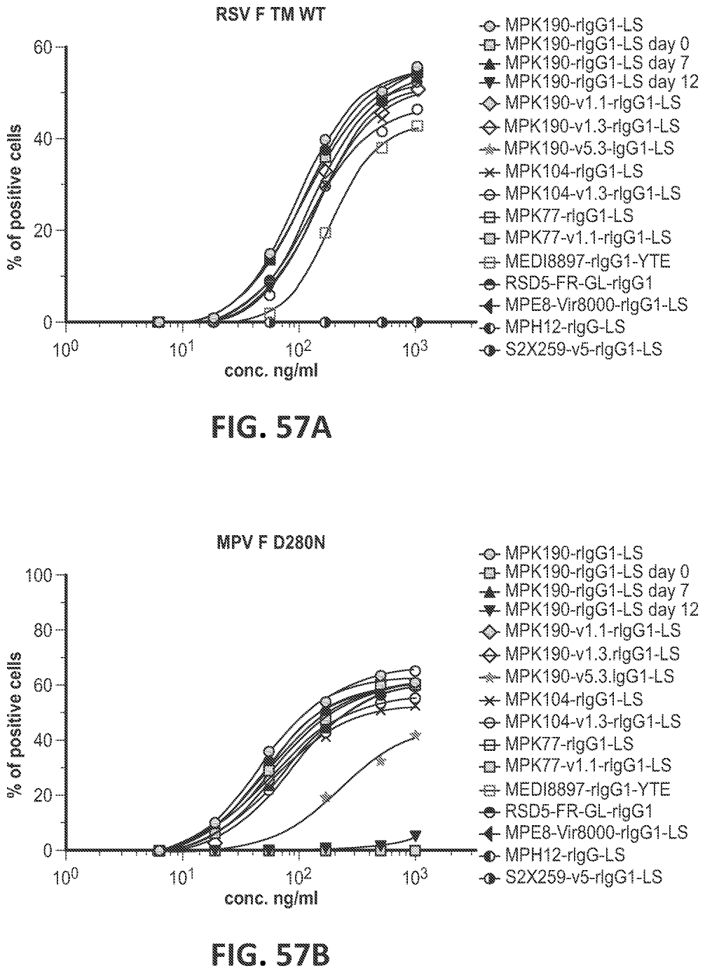

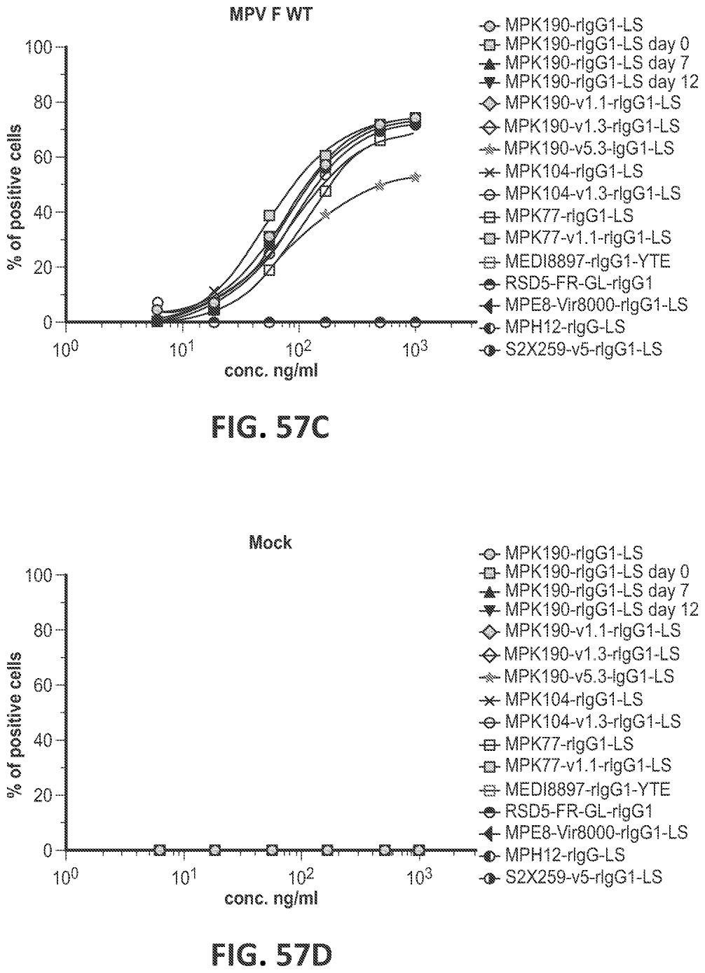

FIGS. 57A-57D show results of binding (FACS) of MPK antibody variants to RSV F TM WT (FIG. 57A), MPV F D280N (FIG. 57B), MPV F (FIG. 57C), and mock binding (FIG. 57D).

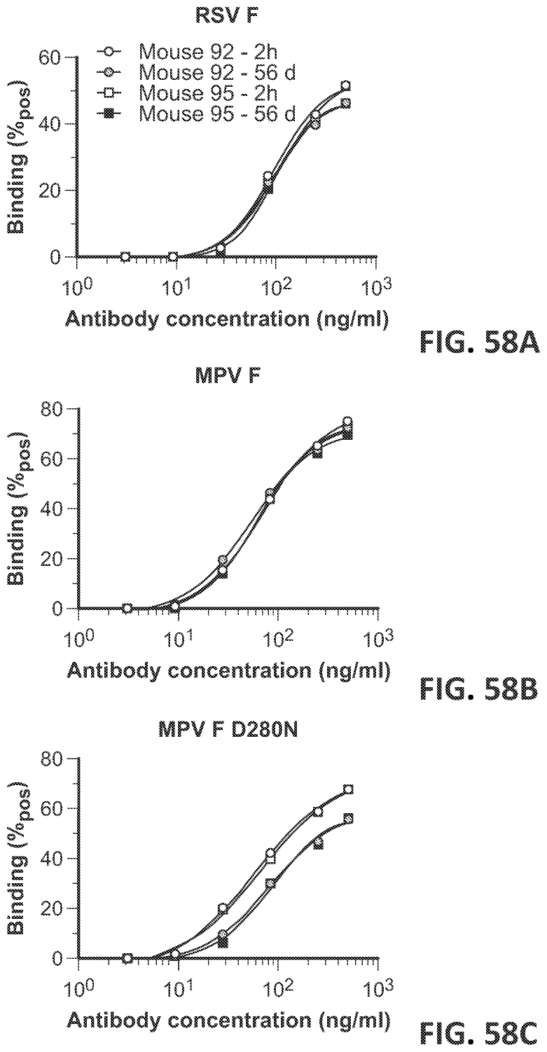

Next, in vivo mouse sera was tested for binding to RSV-F (FIG. 58A), MPV-F (FIG. 58B), and MPV-F D280N (FIG. 58C) expressed on Expi293F transfected cells. Same protocol as above was used. Settings that were used are further detailed in Table 47 and Table 48. Binding to RSV-F and MPV-F was unaltered after more than 50 days. Binding to MPV-F D280N was maintained after in vivo incubation greater than 50 days with minimal loss of binding (EC50 from 62 ng/ml at 2 h to 84 ng/ml at 56 d).

TABLE 47
Mouse sera concentrations 92 and 95 after 2 h and 56 days

| Mouse | | | | | | | | | Mouse | | | | | | | | |
|---|---|---|---|---|---|---|---|---|---|---|---|---|---|---|---|---|---|
| serum | | | conc. ng/mL | | | | | | serum | | | conc. ng/mL | | | | | |
| 92 2 | h | RSV | 500 | 250 | 83.33 | 27.78 | 9.26 | 3.09 | 92 2 | h | MVP F | 500 | 250 | 83.33 | 27.78 | 9.26 | 3.09 |
| 92 56 | d | F | 500 | 250 | 83.33 | 27.78 | 9.26 | 3.09 | 92 56 | d | D280N | 500 | 250 | 83.33 | 27.78 | 9.26 | 3.09 |
| 95 2 | h | | 500 | 250 | 83.33 | 27.78 | 9.26 | 3.09 | 95 2 | h | | 500 | 250 | 83.33 | 27.78 | 9.26 | 3.09 |
| 95 56 | d | | 500 | 250 | 83.33 | 27.78 | 9.26 | 3.09 | 95 56 | d | | 500 | 250 | 83.33 | 27.78 | 9.26 | 3.09 |
| 92 2 | h | MPV | 500 | 250 | 83.33 | 27.78 | 9.26 | 3.09 | 92 2 | h | Mock | 500 | 250 | 83.33 | 27.78 | 9.26 | 3.09 |
| 92 56 | d | F | 500 | 250 | 83.33 | 27.78 | 9.26 | 3.09 | 92 56 | d | | 500 | 250 | 83.33 | 27.78 | 9.26 | 3.09 |
| 95 2 | h | | 500 | 250 | 83.33 | 27.78 | 9.26 | 3.09 | 95 2 | h | | 500 | 250 | 83.33 | 27.78 | 9.26 | 3.09 |
| 95 56 | d | | 500 | 250 | 83.33 | 27.78 | 9.26 | 3.09 | 95 56 | d | | 500 | 250 | 83.33 | 27.78 | 9.26 | 3.09 |

TABLE 48
Antibody concentrations in mouse sera over time

| | Animal | | | | | | | |
|---|---|---|---|---|---|---|---|---|
| | Time Point | Time point | | | | | | |
| Antibody | Conc., μg/mL | 2 h | 6 h | 24 h | d 3 | d 7 | d 10 | d 14 |
| MPK190-rIgG1-LS(4B6) | 91 | 66.77 | 49.11 | 35.82 | 25.29 | 21.93 | 18.54 | 15.59 |
| | 92 | 84.35 | 59.07 | 40.10 | 29.80 | 25.43 | 20.03 | 15.68 |
| | 93 | 62.94 | 48.22 | 34.96 | 25.43 | 23.77 | 18.24 | 14.21 |
| | 94 | 98.72 | 75.73 | 48.38 | 38.61 | 24.50 | 18.45 | 12.42 |
| | 95 | 66.76 | 46.17 | 34.19 | 24.00 | 20.74 | 18.98 | 15.87 |
| | Conc., μg/mL | d 17 | d 21 | d 24 | d 28 | d 35 | d 42 | d 49 |
| MPK190-rIgG1-LS(4B6) | 91 | 13.83 | 10.98 | 8.52 | 7.03 | 5.29 | 3.90 | 2.71 |
| | 92 | 12.80 | 9.28 | 8.75 | 6.42 | 5.18 | 4.36 | 3.07 |
| | 93 | 10.39 | 8.91 | 7.22 | 621 | 4.12 | 3.14 | 2.10 |
| | 94 | 9.52 | 8.05 | 6.05 | 5.44 | 3.76 | 2.85 | 1.92 |
| | 95 | 12.05 | 11.00 | 8.94 | 7.44 | 4.64 | 3.58 | 2.79 |

### Example 29

### MPK190-v1.3 and Effector Functions

Effector functions of MPK190-v1.3 (a MPK190 variant with the NG motif) were investigated.

### Experiment 1: RSV/MPV ADCC Hep2 Infected RSV

The following settings were used.

Conditions— Cell lines: Hep2 adherent infected with 2.5 MOI HRSV A1 (for 18h), LLC-MK2 adherent infected with 5 MOI MPV (for 18h); Effectors: isolated NK cells from fresh blood (HM WBOI11FF), E:T ratio 9:1; Antibodies listed in Table 49, dilutions in AIM-V (50 ug/ml, 5 ug/ml, 0.5 ug/ml, 0.05 ug/ml, 0.005 ug/ml, 0.0005 ug/ml); Virus: RSV A/A2/61, batch 8 (ultracentrifugated), titer: 1.12E+08 TCID50/ml=1.12*0.7×+10E08pfu/ml=0.784×10E08 15 pfu/ml; MPV-GFP1, batch PEG Concentrated, titer: 2×10E08 pfu/ml; Infection medium: MEM+GlutaMAX[+] Earle's (41090-028)+1% FCS+P/S; Complete medium: MEM+GlutaMAX[+] Earle's (41090-028)+10% FCS+P/S; Plate for ADCC: 384 well, clear, round bottom, polypropylene, sterile (Corning, Cat. Nr.: 3656); Plate for reading: 384 well, clear, flat bottom, polystyrene, sterile (Corning, Cat. Nr.: 3701); Tube for blood uptate: BD Vacutainer K2EDTA, 10 ml (BD Biosciences, Cat. Nr.: 367525); Detection Kit: Cytotoxicity Detection Kit (LDH) (Roche; Cat. Nr.: 11644793001); Dilution medium: AIM V MED, liquid (Research Grade) (Life Technologies; Cat. Nr.: 12055091); NK Isolation Kit: MACSxpress WB NK cell isolation kit, human (Miltenyi Biotec, Cat. Nr.: 130-127-695); Antibodies for staining: Anti-CD107 PE (BioLegend, Cat. Nr.: 328608, Clone H4A3, Mouse IgG1, kappa) (use 1.5 ul/well); Anti-CD16 FITC (DAKO Schweiz, Cat. Nr.: F701101, Clone DJ130c, Mouse IgG1, kappa) (use 2.5 ul/well); Anti-CD56 APC (BioLegend, Cat. Nr.: 318310, Clone HCD56, Mouse IgG1, kappa) (use 1.5 ul/well); 4h LDH, 4h FACS; Hep2 adherent infected with 2.5 MOI RSV A2: viability 95% (from 2 infected flasks, recovery of 7.6 Mio cells); LLC-MK2 adherent infected with 5 MOI MPV: viability 95% (from 2 infected flasks, recovery of 4.465 Mio cells); HU_WB011_FF: isolated NK: from 120 ml fresh blood isolated 22.26 Mio cells.

TABLE 49
Antibodies used in RSV/MPV ADCC Hep2 infected RSV

| | | Concentration | |
|---|---|---|---|
| | Batch: | (ug/ml): | Specificity: |
| MPK190-v1.3-rIgG1m17, 1-LS | 20220721/WWU | 1877 | |
| MPK190-v1.3-rIgG1m17, 1-LA | 20220720/851 | 2180 | |
| MPK190-v1.3-rIgG1m17, 1-GRLR | 20220802/GD3 | 1465 | |
| MEDI8897-rIgG1-YTE (nirsevimab) | 20180928/ZAB | 942 | |
| MPE8-rIgG1-LS (Vir8000) | 20180903/WXL | 4774 | |
| S2X259-v5-rIgG1-LS | 20210426/GN4 | 6057 | |
| palivizumab-rIgG1 | 20120326/RCH | 3110 | |
| RSD5-FR-GL-rIgG1 | 20171219/BJU | 4133 | RSV - use on RSV |
| | | | infected cells |
| MPF5 VH117D-rIgG1 (V2) | 20190617/OKI | 2263 | MPV - use on MPV |
| | | | infected cells |
| MPK104-v1.3-rIgG1-LS | 20220512/G2S | 2877 | |
| MPH12-v16-rIgG-LS | 20210813/Y0R | 4611 | |

Procedure—On Day 1 (morning), Hep2 cells and LLC-MK2 cells were washed with PBS, and detached with Trypsin. Trypsin was blocked with medium, and the cells were collected and centrifuged for 4 min at 400 rpm. The supernatant was removed, and the pellet was resuspended in 2 ml. The cells were counted, and plated 2,800,00 cells/flask in a T25 flask in a final volume of 5 ml complete growth medium (2 flasks for each cell type), and incubated until the afternoon. In the afternoon of Day 1, the virus was thawed at 37° C. and virus mixed prepared for both HRSV and MPV, as follows: 3,200,000 supposed number of cells; for HRSV, MOI 2.5, 8.0E+06 particle virus to use, 224 μl virus, 1,536 μl medium; for MPV, MOI 5, 1.6E+07 particle virus to use, 176 μl virus, 1,584 μl medium (already calculated 10% more). The medium was removed from the cells. HRSV virus mix was added to Hep2 cells, and MPV virus mix was added to LLC-MK2 cells, at different MOI: 800 ul inoculum/flask (using growth medium+1% FCS+P/S). After 1 hour, another 4.2 ml of complete medium was added (10% FCS). The cells were incubated for 18h in the incubator.

On Day 2, target cells were prepared by deatching Hep2 and LLC-MK2 cells with trypsin, washing 2 times with AIM-V, then counting the cells and taking an aliquot and adjusting to 326,087 cells/ml (3.77 ml). Effector cells (isolated NK) were prepared by taking blood from EDTA tubes, and isolated NK starting from 120 ml blood with MACSexpress WB NK cell isolation kit, human. This was centrifuged for 1 min at 50×g after 5 minutes of incubation, before placing the tube in a separator. Water EC lysis was made, and the cells were counted and resuspended in AIM-V at 2,934,783 cells/ml. Preparation of target cells: detach Hep2 and LLC-MK2 cells with trypsin; wash 2 times with AIM-V, centrifuge 4 min at 350g; count cells, take an aliquot and adjust to 326,087 cells/ml (7.49 ml).

23 μl target cells/well (7,500 cells/well) in round 384-well-plate was aliquoted (with 16-channel pipet). 23 μl of antibody dilution was added (without mix, changing the tip for every different antibody, with 8-channel pipet). 23 μl/well NK was pipetted in the wells for compensation, except for CD107 (6 wells) (152 μl unstained). All other cells had 0.75 μl/well of anti-CD107 added (239 μl). After 15 min incubation, 23 μl/well of effector cells were added. 23 μl effector cells and 23 μl Trion-X100 was added to the control wells. An additional AIM-V was added in the control wells to reach 69 μl/well final volume. The plate was incubated for 4 hours.

Next, the LDH-substrate was thawed (suspended the substrate with 1 ml water), and flat bottom 384-well-plates were prepared for LDH measurement. The LDH-substrate was centrifuged for 4 min at 420×g. 35 μl of supernatant was transferred for LDH-Assay in the flat bottom 384-well plates without disturbing the cell pellet and without making bubbles. The supernatant for LDH-assay was stored in the fridge until further use.

Air bubbles were removed from the plates, and the LDH-substrate was prepared: 39,37 μl substrate+0.87 μl catalyst per well. 35 μl of LDH-substrate was added to the supernatants. Air bubbles were removed again, then reading performed with the kinetic protocol in an ELISA-reader (490 nm-650 nm).

For FACS analysis, with the pellet of cells (after removing all supernatant), 50 μl/well of PBS-0.1% BSA was added. This was centrifuged for 4 min, 400g. Supernatant was removed, then cells were resuspended vortexing the 96-well plate at about 1800 rpm. Staining mix was made as follows: anti-CD16—1.5 μl/well, 234.3 μl total; anti-CD56—0.75 μl/well, 117.15 μl total; for a total of 351.45 μl; 1991.55 μl PBS-0.1% BSA. Only one sample of each duplicate was stained. 15 μl/well of the mix was added and put at 4° C. for 20 min. Addition of PBS-0.1% BSA, centrifugation, removal of supernatant, and resuspension of the cells was repeated. 80 μl/well of PBS-0.1% BSA was then added, and the plate was measured in FACS Canto (Software FACS Diva).

Conclusions—As a result, the MPV infected cells were completely flat. Analysis with CD107 overexpression showed differences among the antibodies. In both cases (RSV and MPV) there was a negative correlation between CD16 downregulation and CD107 overexpression, and in case of LDH there was a direct correlation (as expected) between LDH release and CD107 overexpression.

Experiment 2: RSV/MPV ADCC Promega Hep2 infected RSV and LLC-MK2 infected MPV. The following settings were used:

Conditions— Target Cells: Hep2 adherent infected with 2.5 MOI HRSV A2, LLC-MK2 adherent infected with 5 MOI MPV, Cells infected in Exp ID 16937, 12,500 cells/well, E:T 6:1; ADCC Bioassay Effector Cells: Cell needed 75,000 cells/well, V158 variant; ADCC Assay Buffer: RPMI1640+4% Low IgG Serum; Dilution Buffer: ADCC Assay Buffer; Antibodies listed in Table 49; Antibody dilution: dilution in ADCC Assay Buffer, 3× final concentration, start at 10000 ng/ml, 1:4, 9 concentrations; Plate: white, flat-bottom 96-well assay plates (PerkinElmer, Cat. Nr.: 6005680); Kit: ADCC Reporter Bioassay, Core Kit 5× (Promega, Cat. Nr.: G7018); 23 h assay; Hep2 adherent infected with 2.5 MOI RSV A2: viability 95% (from 2 infected flasks, recovery of 7.6 Mio cells); LLC-MK2 adherent infected with 5 MOI MPV: viability 95% (from 2 infected flasks, recovery of 4.465 Mio cells).

Protocol— Day 1: ADCC Assay Buffer: Thaw the low IgG serum in a 37° C. water bath; Add 1.5 ml low IgG serum to 36 ml of RPMI 1640 medium to make 37.5 ml ADCC Assay Buffer; mix well and warm to 37° C. prior to use. Preparation of target cell—Collect target cells; wash them once with PBS (add 4 ml PBS, centrifuge 4 min at 400g); count cells and take the needed aliquot; Centrifuge and resuspend cells in ADCC Assay Buffer, adjusting to E:T ratio 6:1, 0.5 Mio cells/ml, 3.03 ml each; Pipet 25 ul/well of target cells in the inner wells of a white 96 well assay plates; Dispense 75 μl of ADCC Assay Buffer into outermost wells; Cover plates with lids and keep the plates on the bench before adding antibody dilutions and ADCC Bioassay Effector Cells; Prepare antibody dilution; Add 25 ul/well of antibody dilution series to the white plate already containing target cells' Cover plates with lids and keep the plates on the bench for 25 min at RT before adding ADCC Bioassay Effector Cells at the next step; Add 7.2 ml of ADCC Assay Buffer (prewarmed to 37° C.) to a 15 ml falcon tube; Thaw 2 vials of ADCC Bioassay Effector Cells in a 37° C. water bath until cells are just thawed; Gently mix the cell suspension by pipetting 1 or 2 times; Transfer 2×630 ul of cells to the falcon tube containing 7.2 ml of ADCC Assay Buffer; Mix well by gently inverting the tube 2 times; Pipet 25 ul/well of cells to the inner wells of the 96-well assay plates already containing target cells and antibody; Cover plates with lids, and incubate the plates for 6-20 hours at 37° C. in a humidified CO2 incubator; Do not stack plates within the incubator and thaw the Bio-Glo™ together with the incubation; Thaw the Bio-Glo™ Luciferase Assay Buffer in a refrigerator overnight or in a room temperature water bath on the day of the assay; Equilibrate BioGlo™ Luciferase Assay Buffer and BioGlo™ Luciferase Assay Substrate to RT protected from light; Transfer the buffer into the amber bottle containing Substrate and mix by inversion until the Substrate is thoroughly dissolved; Store reconstituted Bio-Glo™ Luciferase Assay Reagent at RT, protected from light; Protocol— Day 2: Adding Bio-Glo™ Luciferase Assay Reagent; Remove assay plates from the 37° C. incubator and equilibrate to RT for 15 minutes (equilibrate also Bio-Glo™ Reagent at RT); Add 75 ul/well of Bio-Glo™ Luciferase Assay Reagent to all the inner 60 wells of the assay plates (avoid creating any bubbles); Add also 75 ul/well of Bio-Glo™ Luciferase Assay Reagent to well B1, C1 and D1 in each assay plate to determine plate background; Incubate at RT for 15 min; Measure luminescence (integration time should be 0.5 sec/well) using a plate read with glow-type luminescence read capabilities after 15 min; Use Synergy 2 Fluorimeter (Promega kit Luminescence Protocol).

### Experiment 3: RSV/MPV ADCP Promega Hep 2 Infected RSV and LLC-MK2 Infected MPV

Protocol— Day 1: Assay Buffer: Thaw the Low IgG Serum in a 37° C. bath; Add 1.5 ml Low IgG Serum to 36 ml of RPMI 1640 Medium to make 37.5 ml of Assay Buffer for two assay plates: mix well and warm to 37° C. prior to use; Preparation of target cell—Collect target cells; Wash them once with PBS (add 4 ml PBS, centrifuge 4 min at 400g); Count cells and take the needed aliquot; Centrifuge and resuspend cells in Assay Buffer, adjusting to E:T ratio 5:1, 0.4 Mio cells/ml, 3.03 ml each; Pipet 25 ul/well of target cells in the inner wells of a white 96 well assay plates; Dispense 75 μl of Assay Buffer into outermost wells; Cover plates with lids and keep the plates on the bench before adding antibody dilutions and ADCP Bioassay Effector Cells; Prepare antibody dilution; Add 25 ul/well of antibody dilution series to the quite plate already containing target cells; Cover plates with lids and keep the plates on the bench for 30 min at RT before adding ADCP Bioassay Effector Cells at the next step; Add 7.14 ml of Assay Buffer (prewarmed to 37° C.) to a 15 ml falcon tube; Thaw 2 vials of ADCP Bioassay Effector Cells in a 37° C. water bath until cells are just thawed; Gently mixt eh cell suspension by pipetting 1 or 2 times; Transfer 2×550 ul of cells to the falcon tube containing 7.14 ml of Assay Buffer; Mix well by gently inverting the tube 2 times; Pipet 25 ul/well of cells to the inner wells of the 96-well away plates already containing target cells and antibody; Cover plates with lids, and incubate the plates for 6-20 hours at 37° C. in a humidified CO2 incubator; Do not stack plates within the incubator and thaw the Bio-Glo™ together with the incubation; Thaw the Bio-Glo™ Luciferase Assay Buffer in a refrigerator overnight or in a room temperature water bath on the day of the assay; Equilibrate BioGlo™ Luciferase Assay Buffer and BioGlo™ Luciferase Assay Substrate to RT protected from light; Transfer the buffer into the amber bottle containing Substrate and mix by inversion until the Substrate is thoroughly dissolved; Store reconstituted Bio-Glo™ Luciferase Assay Reagent at RT, protected from light; Protocol— Day 2: Adding Bio-Glo™ Luciferase Assay Reagent; Remove assay plates from the 37° C. incubator and equilibrate to RT for 15 minutes (equilibrate also Bio-Glo™ Reagent at RT); Add 75 ul/well of Bio-Glo™ Luciferase Assay Reagent to all the inner 60 wells of the assay plates (avoid creating any bubbles); Add also 75 ul/well of Bio-Glo™ Luciferase Assay Reagent to well B1, C1 and D1 in each assay plate to determine plate background; Incubate at RT for 15 min; Measure luminescence (integration time should be 0.5 sec/well) using a plate read with glow-type luminescence read capabilities after 15 min; Use Synergy 2 Fluorimeter (Promega kit Luminescence Protocol).

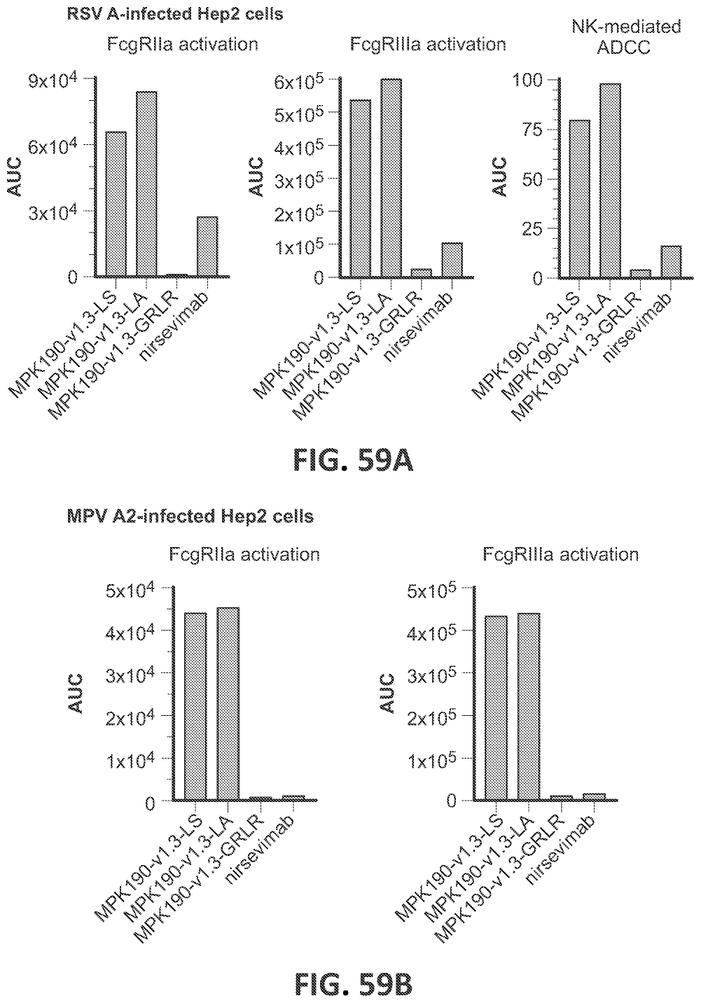

Conclusions—MPK190 v1.3 with both LS and LA showed higher activation of FcγRIIa (ADCP) and FcRγIIIa (ADCC) and induce better NK cell killing (ADCC) than nirsevimab, in RSV A-infected Hep2 cells (FIG. 59A) and in MPV A1-infected Hep2 cells (FIG. 59B).

### Example 30

### Mpk190-v1.3, Mpk65-v2, Mpk176, and Mpk201 In Vivo Testing

MEDI18897-rlgG1m17,1, MPK190-v1.3-rlgG1m17,1, MPE8-v3-rlgG1, or positive control antibody Palivizumab were administered to test groups of mice at doses of 2.0 or 0.5 mg/kg, and the mice were infected with RSV. Mice used for this study were BALB/c mice, female, and between 7-9 weeks of age. Antibodies were diluted according to the master excel using sterile 1×PBS. Dilutions were kept on ice until administration. Mice were weighed, restrained in a plastic restrainer, briefly heated under a heat lamp, and compound was administered intravenously via tail vein injection. Mice were dosed by weight, receiving 10 uL of the corresponding compound dilution per gram body weight (ex. a 20 g mouse would receive 200 uL). Mice were observed post-infection to confirm health.

On the day of infection (DO), blood was collected from each mouse to confirm antibody concentration. A small volume of blood was collected via cheek bleed into SST tubes and spun at 2000×g for 10 minutes at 4° C. Serum was removed, transferred to microcentrifuge tubes, and stored at −20° C. On DO, mice were dosed intranasally under isoflurane anesthesia. Virus was transported to the vivarium on dry ice and inoculums were diluted immediately before use. Inoculums were generated based on calculations in the master excel sheet and kept on ice.

Mice were infected with lethal dose: 3E6 pfu/mouse of MaRSV. Mice were placed in induction chamber with 4% isoflurane and 1.00 L/min 02. Once unresponsive to toe pinch, 50 μL of inoculum was delivered to each mouse intranasally and animals were observed until recovery. Stock virus and inoculum were titered via plaque assay to confirm accurate dilution.

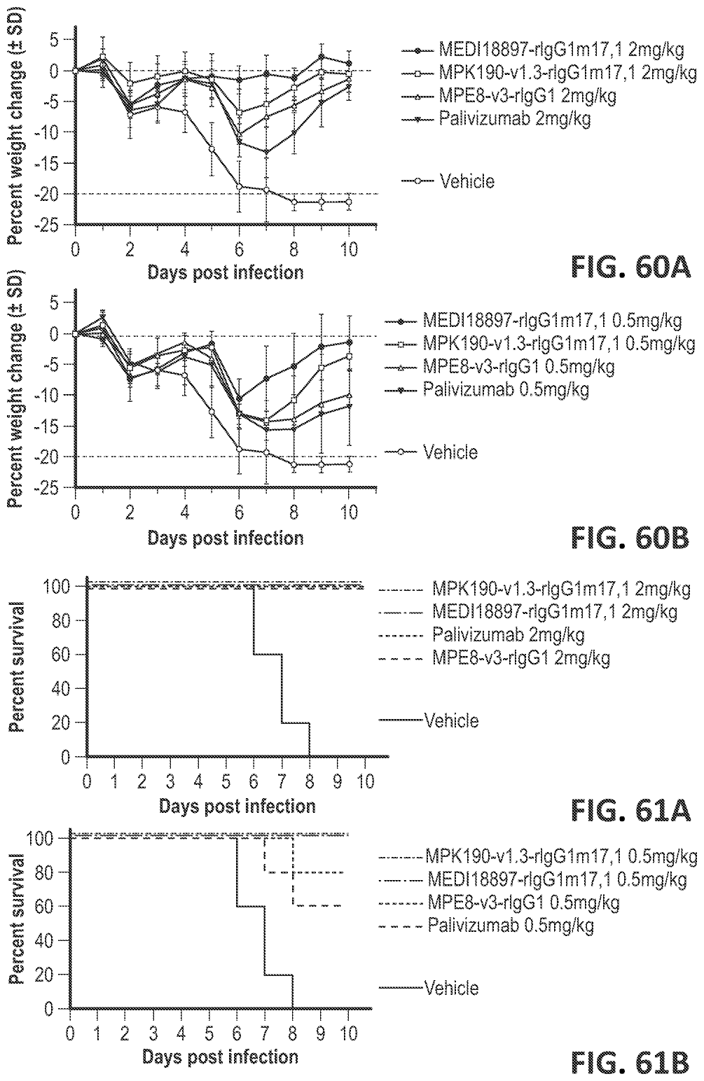

Weight loss and morbidity were monitored on Days 1-10. Results are presented in FIG. 60A (weight loss, dose 2 mg/kg), FIG. 60B (weight loss, dose 0.5 mg/kg), FIG. 61A (morbidity, dose 2 mg/kg), and FIG. 61B (moribidity, dose 0.5 mg/kg). MPK190-v1.3 protects mice from lethal infection with RSV. A 2 kg/mg dose fully protected mice from morbidity and death.

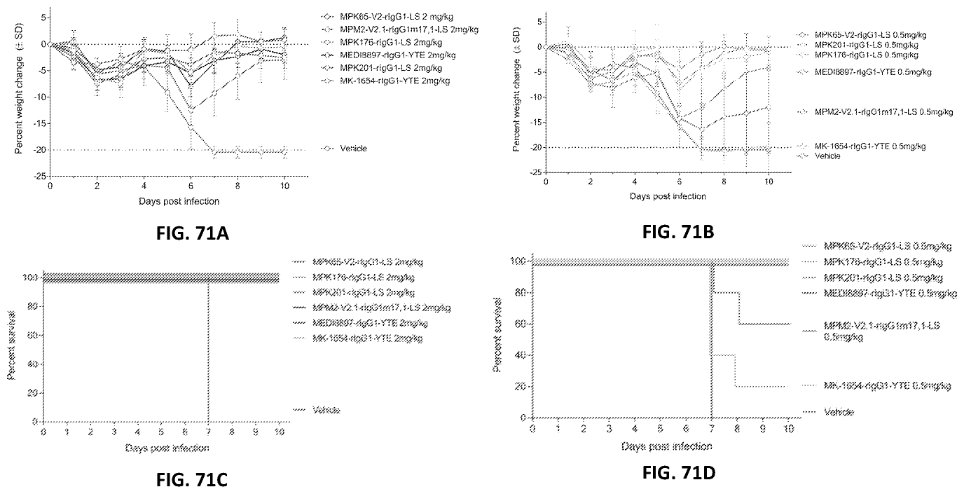

In vivo tests in mice similar to those performed for MPK190-v1.3 were performed using MPK65-v2, MPK176, and MPK201. Test doses were 2 mg/mg (FIG. 71A and FIG. 71C) or 0.5 mg/kg (FIG. 71B and FIG. 71D). Mice treated with MPK65-v2, MPK176, and MPK201 had less weight loss than MEDI8897 and MK-1654 at the same doses. MPK65-v2, MPK176 and MPK201 provide better protection against weight loss at 0.5 mg/kg than MPM2-v2.1, MEDI8897 (nirsevimab) and MK-1654 (clesrovimab).

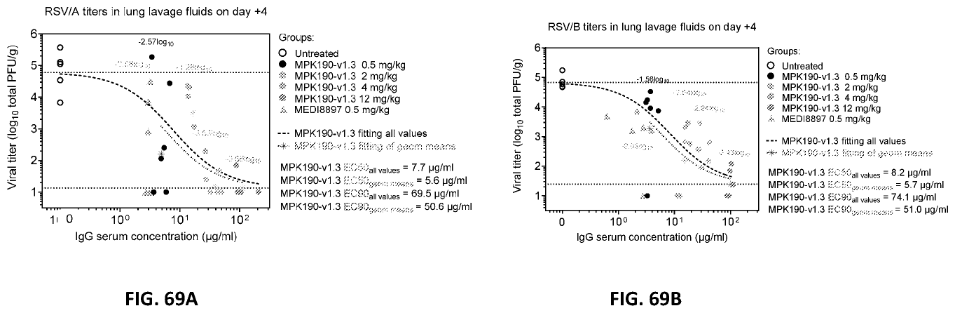

In vivo studies were also performed in cotton rats to assess the ability of MPK190-v1.3 to protect against infection by RSV A (FIG. 69A) and RSV B (FIG. 69B). Antibodies were administered by IP injection for RSV A rats and IM injection for RSV B rats 24 hours before challenge with either RSV/A/GA1/Tracy-CR (1989) or RSV/B/18537-CR (1962). Samples were taken a day 4 post challenge and viral titers were assess in lung lavage fluids. Serum levels of 8+0.2 g/ml MPK190-v1.3 (EC50), corresponding to a dose of about 2 mg/kg, significantly reduced the viral titer by 1.78+0.05 log 10 (about 98% reduction compared to baseline). A 2-log 10 (99%) viral reduction can be achieved with 10.4+1.1 g/ml MPK190-v1.3.

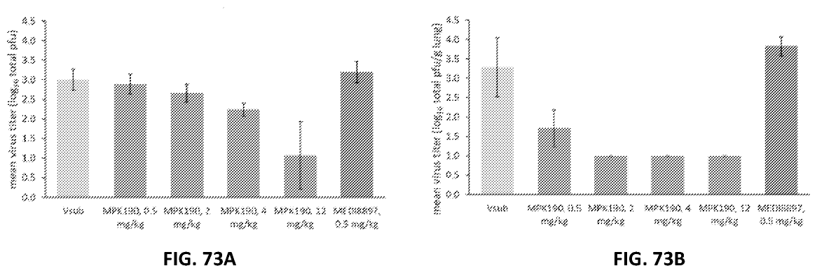

Similar in vivo studies in cotton rats were performed using MPV. Antibodies were administered by IM injection 24 hours prior to viral challenge. Viral titers in lung lavage fluids (FIG. 73A) and nasal lavage fluids (FIG. 73B) were assessed. MPK190-v1.3 showed a protective effect in the lungs across all doses tsted. EC50 is expected to be <3 μg/ml in human patients.

### Example 31

### RSV-Only Antibodies and Neutralization of RSV-A and RSV-B

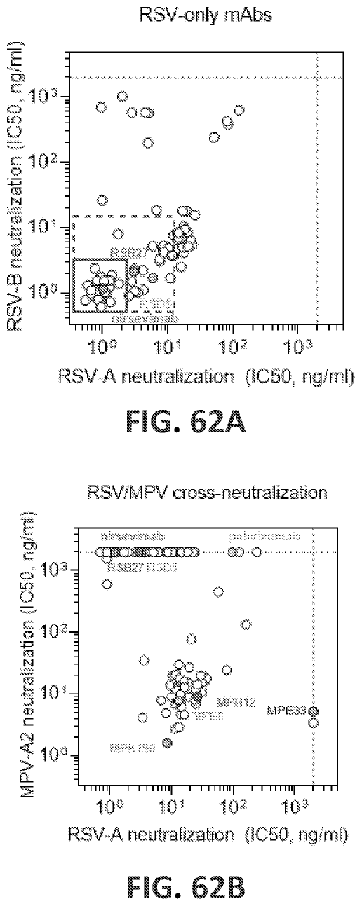

FIG. 62A shows results of neutralization screenings from 6 donors of RSV-only antibodies, plotting RSV-A against RSV-B IC₅₀. 82 hits were identified. 40 antibodies showed high neutralization potency against RSV-A and RSV-B (IC50<15 ng/ml). These antibodies belong to different clonal families (use>20 different VJ gene pairs). 20 antiboides showed the highest neutralization (IC50<2.5 ng/ml) similar to nirsevimab (IC50=1.0 and 1.1 ng/ml). These 20 antiboides blond to 2 clonal families: 18 mAbs are clonally related (VJ gene usage: VH5-51/VK3-15), and 2 mAbs are clonally related (VJ gene usage: VH1-24/VK2-28).

Data on 2 mAbs belonging to 2 different clonal families showed that they bind to RSV-A with high affinity (KD<10⁻¹⁰ M), they compete with D25 (parental of nirsevimab) and RSD5 for binding to site 0 of the RSV F protein, they neutralize different RSV A and B strains similarly to nirsevimab, and they show very low levels of activation of FcgRIIa and IIIa, suggesting poor effector functions. Table 50 shows IC50 values of select RSV antibodies against RSV A and RSV B strains.

TABLE 50
IC50 of select RSV antibodies against
RSV A, RSV B, RSV A2, and RSV B1

| mAb IC50 | RSV A | RSV B | RSV A2 | RSV B1 |
|---|---|---|---|---|
| (ng/ml) | Randall | 9719/09 | (BEI) | (BEI) |
| MPK44 | 2.174 | 1.662 | 0.1 | 0.7 |
| MPK65-v2 | 1.701 | 0.7406 | 0.5 | 0.4 |
| RSD5 | 6.654 | 0.7649 | 0.8 | 1.0 |
| MEDI8897 | 2.419 | 1.254 | 0.2 | 0.4 |
| Palivizumab | 122.1 | 78.61 | not tested | not tested |

As expected, RSV-only antibodies did not show neutralization of MPV-A2 in a similar neutralization screening plotting MPV-A against RSV-A (FIG. 62B).

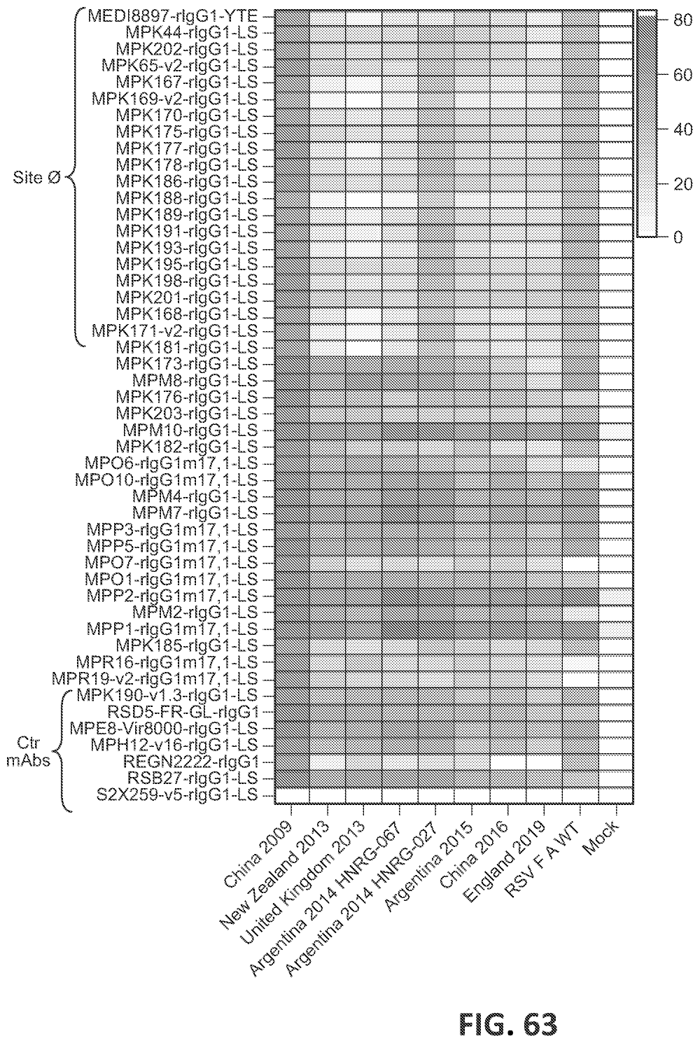

FIG. 63 shows a heat map of RSV-only antibodies and their binding to a panel of F proteins of recently circulating RSV B strains. 30 out of 40 antibodies maintained binding to F protens. Half of expected Site 0 mAbs (n=10) and most of unmapped mAbs showed better breadth compared to nirsevimab.

The abilities of MPK65-v2, MPK176, and MPK201 to bind various RSV known to be escape mutants for nirsevimab, are summarized in Table 51. Mutations are indicated as compared to RSV-B B18537/62.

TABLE 51
Binding of nirsevimab escape mutants

| | RSV-B | | L172Q + | | | | RSV-B |
|---|---|---|---|---|---|---|---|
| mAb | B18537/62 | L172Q | S173L | P312H | S186R | L203I | B9320 |
| MPK176-rIgG1-LS | 41.6 | 42.6 | 34.3 | 37.8 | 33.9 | 44 | 41.6 |
| MPK201-rIgG1-LS | 46.8 | 46.2 | 37.7 | 41.2 | 37.6 | 50.2 | 42.6 |
| MPK65-v2-rIgG1-LS | 54.2 | 56.2 | 50 | 51.4 | 50.3 | 60.4 | 56.1 |
| MED18897-rIgG1-YTE | 20.7 | 22.9 | 16.1 | 14.5 | 15 | 0.12 | 37.5 |

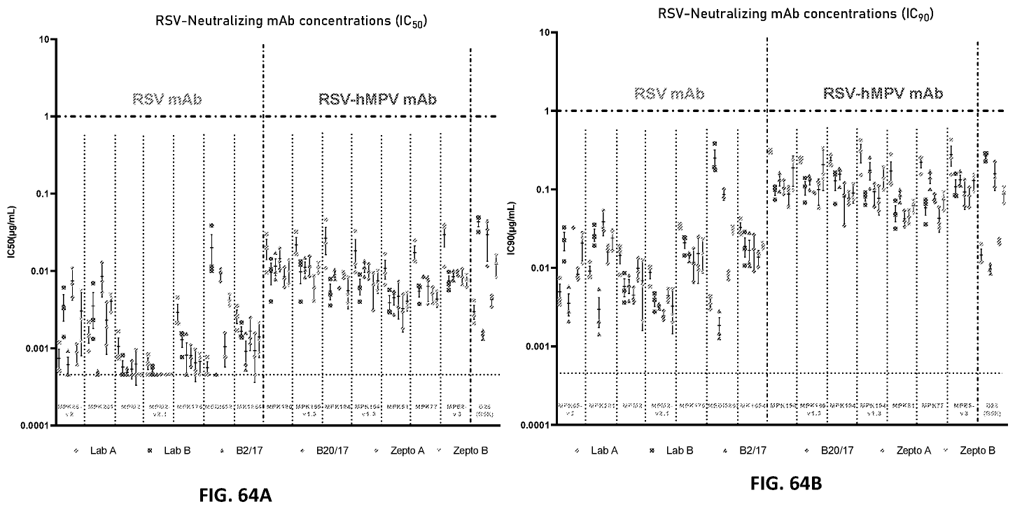

Further testing of MPK201 and MPK176 neutralization against various RSV A and RSV B lab-adapted and circulating strains was conducted (FIGS. 64A (IC50) and 64B (IC90)) and demonstrate neutralization breadth of these antibodies.

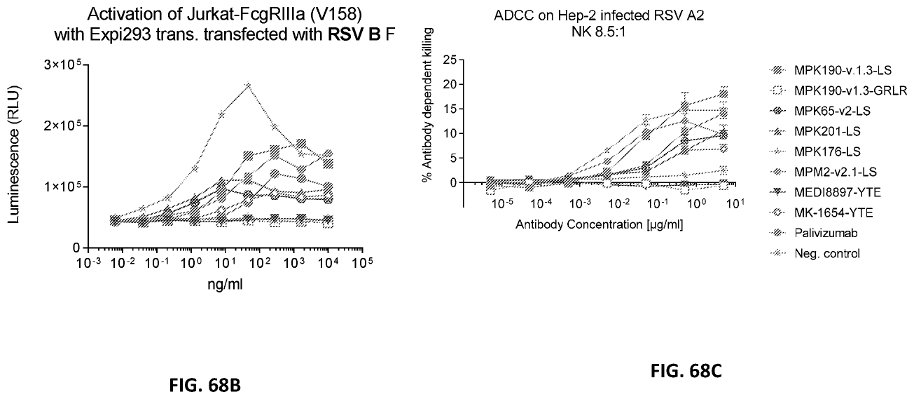

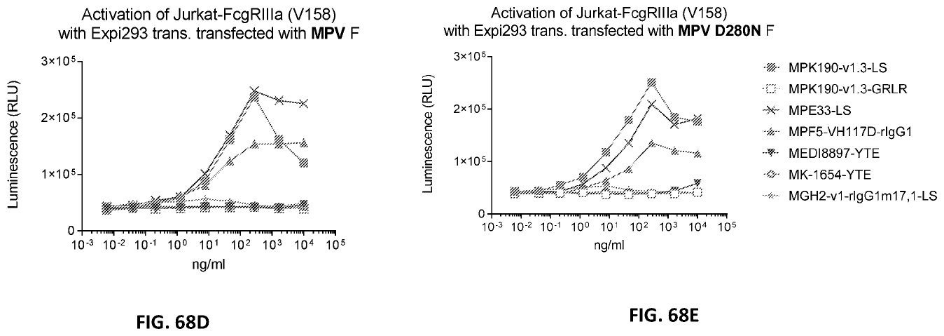

Further testing of MPK201, MPK176, and MPK65-v2 effector function using various RSV and MPV strains was also tested (FIG. 68A-68C). The tested antibodies showed partial activation of FcγRIIa (ADCP) and FcRγIIIa (ADCC) and induce NK cell killing (ADCC) at high concentrations. The RSV-only antibodies had lower effector functions than MPK190-v1.3, but higher effector functions than nirsevimab, which had no detectable effector functions in these assays.

The abilities of MPK65-v2, MPK176, and MPK201 to neutralize various RSV strains are summarized in Table 52. Results are IC50 in ng/ml. “RSV A” and “RSV B” are screening GFP strains. All other strains are non-GFP commercial strains.

TABLE 51
Neutralization of RSV strains

| | Geo | | | A | | | A2 | A2 | A | |
|---|---|---|---|---|---|---|---|---|---|---|
| | mean | RSV | RSV | 2001/ | A1997/ | A1998/ | BPR- | VR | 2000/ | Ds |
| mAb | IC50 | A2 | B | 3- 12 | 12-35 | 3-2 | 344-0 | 1540 | 3-4 | Red 5 |
| MPK176-rIgG1-LS | 2.5 | 4.149 | 2.185 | 2.12 | 2.08 | 2.45 | 5.04 | 2.87 | 2.3 | 1.06 |
| MPK201-rIgG1-LS | 2.0 | 2.832 | 1.485 | 1.77 | 2.33 | 3.01 | 4.92 | 1.53 | 1.26 | 1.23 |
| MPK65-v2- rIgG1-LS | 0.8 | 0.9551 | 0.6066 | 0.59 | 0.56 | 0.17 | 1.85 | 0.95 | 0.74 | 0.47 |
| MED18897- rIgG1-YTE | 0.8 | 1.016 | 1.136 | 0.63 | 0.76 | 0.90 | 1.01 | 1.14 | 0.57 | 0.48 |

The binding affinities and effector functions of MPK65-v2, MPK176, and MPK201 for RSV are summarized in Table 53.

TABLE 53
Binding affinities and effector functions

| | Affinity | | Effector functions | |
|---|---|---|---|---|
| | RSV-A F | RSV-BF | Pseudo | Pseudo |
| mAb | (KD, M) | (KD, M) | ADCC | ADCP |
| MPK176- rIgG1-LS | 5.911E−11 | 1.00E−12 | 92838 | 14235 |
| MPK201- rIgG1-LS | 1.00E−12 | 1.00E−12 | 42981 | 6545 |
| MPK65-v2- rIgG1-LS | 1.345E−10 | 4.042E−10 | 60729 | 12745 |
| MED18897- rIgG1-YTE | 1.00E−12 | 1.224E−09 | 29227 | 2732 |

IC50 (ng/mL) for neutralization of RSVA, binding KD (M) for RSVA F (assessed by BLI), and RSV F A (% binding, FACS) for several RSV strains (mutations indicated with reference to ref A2 sequence) using MPK65-v2, MPK176, and MPK201 variants as well as compartors MPK190v1.3-rIgG1m17,1-LS, MED18897-iIgGl-YTE, and MK-1654-rIgG1m17,1-YTE was assessed. Results are presented in Table 54.

TABLE 54
RSV A Neutralization and Binding Data

| | | A2 | N208Y- | | S433P- | S443P- | | | |
|---|---|---|---|---|---|---|---|---|---|
| mAb | IC50 | Ref | N671I | S433P | G446E | K445N | G446E | L203I | KD |
| MPK65-v2-v1.2 | 4.376 | 56 | 0.38 | 31.7 | 32.9 | 33.5 | 25.4 | 18 | 2.054E−10 |
| MPK65-v2-v3.1 | 5.401 | 53 | 15.1 | 34.4 | 34.6 | 33.4 | 26.9 | 4.74 | 1.915E−10 |
| MPK201-v1.2 | 2.879 | 66.9 | 0.027 | 35.9 | 36.4 | 36 | 29 | 40 | 2.109E−10 |
| MPK201-v4.1 | 4.353 | 54.1 | 0.021 | 26.4 | 28.2 | 2703 | 18 | 13.1 | 2.925E−10 |
| MPK176-v1.3 | 3.936 | 78.1 | 24.5 | 4.98 | 0.33 | 0.036 | 0.017 | 56.1 | 1.358E−10 |
| MPK174-v4.3 | 4.346 | 78.1 | 19.1 | 4.41 | 0.33 | 0.041 | 0.019 | 56.6 | 1.389E−10 |
| MPK190-v1.3 | 15.29 | 80.8 | 59.6 | 47.9 | 52.5 | 44 | 60.7 | 61.7 | NA |
| MED 18897 | 7.737 | 60.2 | 0.049 | 35.3 | 35.8 | 34.5 | 28.5 | 0.04 | 3.5E−11 |
| MK-1654 | 4.605 | 83.7 | 43.1 | 7.14 | 1.04 | 0.11 | 0.049 | 62.6 | NA |

Binding KD (M) for RSV B F (assessed by BLI), and RSV F B (% binding, FACS) for several RSV strains (mutations indicated with reference to ref B sequence) using MPK65-v2, MPK176, and MPK201 vaniants as well as compartors MPK190v1.3-rIgG1m17,1-LS, MED18897-iIgG1-YTE, and MK-1654-rIgG1m17,1-YTE was assessed. Results are presented in Table 55.

TABLE 55
RSV B Neutralization and Binding Data

| | | K68N- | K68N- | | | | | |
|---|---|---|---|---|---|---|---|---|
| mAb | B Ref | N201S | N208S | N201S | N208D | N208S | S443P | KD |
| MPK65-v2-v1.2 | 56.4 | 30.8 | 0.047 | 35.9 | 0.47 | 0.041 | 33.8 | 5.807E−10 |
| MPK65-v2-v3.1 | 60.1 | 22.6 | 0.052 | 31.3 | 0.14 | 0.029 | 30.6 | 6.393E−10 |
| MPK201-v1.2 | 61.3 | 47.3 | 12.1 | 47.7 | 0.015 | 14.4 | 45.8 | 1.609E−10 |
| MPK201-v4.1 | 51.9 | 30 | 0.048 | 33 | 0.018 | 0.016 | 31.5 | 5.519E−10 |
| MPK176-v1.3 | 39.9 | 60 | 57.9 | 60.2 | 53.6 | 60.4 | 33.8 | 2.559E−11 |
| MPK174-v4.3 | 34.2 | 60.4 | 59.3 | 60.6 | 5306 | 61.2 | 29.8 | 1.173E−11 |
| MPK190-v1.3 | 72.1 | 63.3 | 61.5 | 63.1 | 60.5 | 63.4 | 66.7 | NA |
| MED 18897 | 57.3 | 0.056 | 0.031 | 27.3 | 0.026 | 0.035 | 38.5 | 2.211E−10 |
| MK-1654 | 63.4 | 66.7 | 65.7 | 64.7 | 63.1 | 64.3 | 22.6 | NA |

Pharmacokinetics of MPK190-v1.3-LS and additional RSV-only antibodies was investigated in SCID mice injected with an IV injection at the indicated doses. Results are presented in Table 56.

TABLE 56
PK Data for SCID Mice

| | | Half-life | Cavg | | CLₛₛ | AUCₗₐₛₜ |
|---|---|---|---|---|---|---|
| | Dose | (day) | (ug/ml) | V_{z} (ml/kg) | (ml/h/kg) | (day*ug/ml) |
| MPK190-v1.3 | 93.750 ± 4.787 | 15.892 ± 0.508 | 56.126 ± 2.690 | 38.307 ± 1.722 | 1.671 ± 0.055 | 735.090 ± 99.455 |
| MPK176 | 93.750 ± 4.787 | 16.080 ± 0.751 | 65.114 ± 6.163 | 35.088 ± 0.818 | 1.516 ± 0.110 | 884.685 ± 116.737 |
| MPK65-v2 | 97.200 ± 6.573 | 12.839 ± 1.237 | 64.723 ± 5.872 | 27.919 ± 3.257 | 1.509 ± 0.135 | 808.720 ± 79.347 |
| MPK201 | 95.000 ± 7.906 | 9.561 ± 0.220 | 46.008 ± 5.606 | 29.055 ± 6.311 | 2.101 ± 0.410 | 39.996 ± 34.778 |

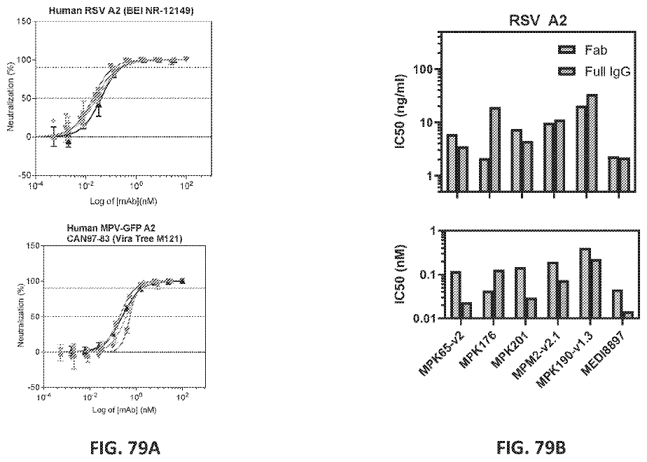

Fab fragments of the full-length antibodies were tested (FIG. 79A and FIG. 79B) and neutralization data was similar to that obtained using full-length antibodies.

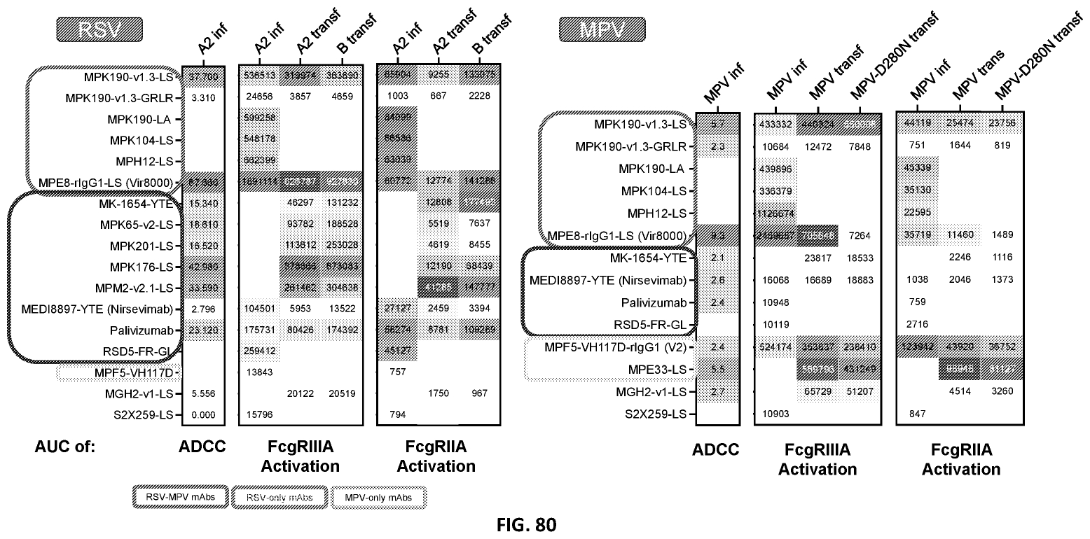

Effector functions of various RSV-only antibodies are summarized in FIG. 80.

A summary of the properties of antibodies for which combinations with were tested under forced stress conditions s provided in Table 57. 2,2′-azobis (2-amidinopropane) dihydrochloride (AAPH) was used in some tests as an oxidative stress reagent.

TABLE 57
MPK65-v2, MPK176, and MPK201 Variant Summaries

| | | Post stress | |
|---|---|---|---|
| | | neutralization | Post stress RSV B F |
| mAb | Post stress affinity | potency | protein binding |
| MPK176-v1.3 | High affinity to RSV F | Potent neutralization | Decrease in binding for |
| (site IV) | A and RSV F B after | capability maintained, | mutant S443P (low |
| | stress conditions | despite less | frequency <1%) and |
| | maintained | neutralization potency | China 2014 strain after |
| | | for RSV A and B after | oxidative stress with |
| | | oxidative stress with | 1 mM AAPH |
| | | 1 mM AAPH | |
| MPK176-v4.3 | High affinity to RSV F | Potent neutralization | Decrease in binding for |
| (site IV) | A and RSV F B after | capability maintained, | mutant S443P (low |
| | stress conditions | despite less | frequency <1%) and |
| | maintained | neutralization potency | China 2014 strain after |
| | | for RSV A and B after | oxidative stress with |
| | | oxidative stress with | 1 mM AAPH |
| | | 1 mM AAPH | |
| MPK201-v1.2 | High affinity to RSVF | Potent neutralization | Loss binding for |
| (site Ø) | A and RSV F B after | capability maintained | mutant N208S |
| | stress conditions | | (low frequency <1%), |
| | maintained | | after stress in |
| | | | phosphate buffer |
| MPK201-v4.1 | High affinity to RSV F | Potent neutralization | Complete loss binding |
| (site Ø) | A and RSV F B after | capability maintained, | for mutant N208S |
| | stress conditions | despite slightly less | (low frequency <1%) |
| | maintained | neutralization potency | |
| | | for RSV A after | |
| | | oxidative stress | |
| MPK65-v2-v1.2 | High affinity to RSV F | Slightly less | Loss binding for |
| (site Ø) | A and RSV F B after | neutralization potency | mutant N208S |
| | stress conditions | for RSV A after | (low frequency <1%) |
| | maintained | oxidation | |
| MPK65-v2-v1.2 | High affinity to RSV F | Potent neutralization | Loss binding for |
| (site Ø) | A and RSV F B after | capability maintained | mutant N208S |
| | stress conditions | | (low frequency <1%), |
| | maintained | | moreover some |
| | | | reduction in binding |
| | | | observed after |
| | | | oxidative stress |

### Example 32

### Further Characterization of Mpk190-V1.3

Neutralization screenings similar to those or Example 31 were conducted using RSV-only antibodies and MPV/RSV antibodies, including MPK190-v1.3 and several comparator antibodies, with a MPV A strain and a RSV A strain as targets. Cross-neutralization results are presented in FIG. 62B.

Further testing of MPK190-v1.3 and various comparator antibody neutralization against various RSV A and RSV B lab-adapted and circulating strains was conducted (FIGS. 64A (IC50) and 64B (IC90)) and demonstrate neutralization breadth of MPK190-v1.3.

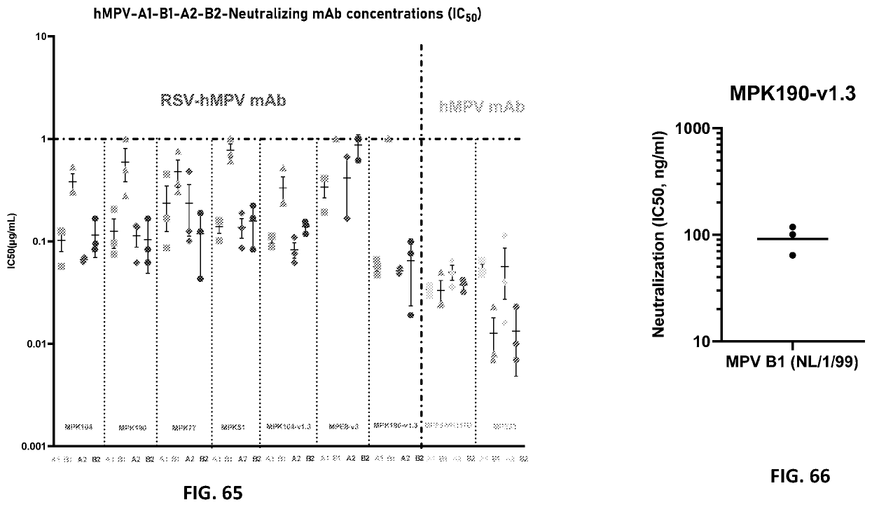

Neutralization testing of MPK190-v1.3 and various comparator antibodies against representatives of all four MPV subtypes, MPV A1, MPV A2, MPV B1, and MPV B2 demonstrated the ability of MPK190-v1.3 to neutralize all four subtypes (FIG. 65).

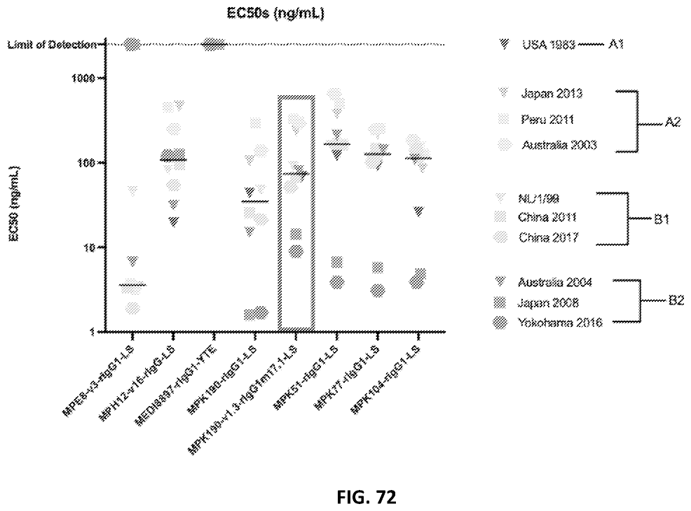

MPK190-v1.3 neutralization of a representative MPV B1 strain virus (NL/1/99) was conducted using a VSF pseudotyped system as in the prior examples (FIG. 66). Neutralization was observed at an average IC50 of 100 ng/ml. Further results with additional MPV strains are provided in FIG. 72, which shows that MPK190v1.3-LS is slightly less potent than MPK190-LS, but very comparable

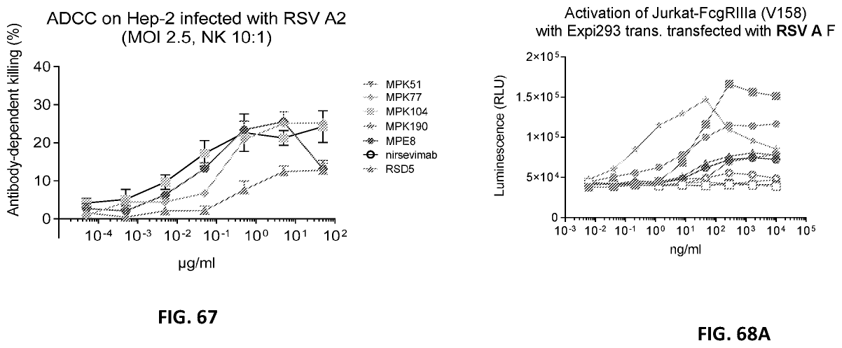

ADCC testing using Hep-2 cells infected with the RSV A2 strain (MOI 2.5, NK 10:1) was conducted and results are presented in FIG. 67, further demonstrating the effector functions of MPK 190.

ADCC testing using Hep-2 cells infected with the RSV A2 strain (MOI 2.5, NK 10:1) was conducted and results are presented in FIG. 67, further demonstrating the effector functions of MPK 190.

Further testing of MPK190-v1.3 effector function using various RSV and MPV strains was also tested (FIGS. 68A-68E). MPK190-v1.3 showed higher activation of FcγRIIa (ADCP) and FcRγIIIa (ADCC) and induces better NK cell killing (ADCC) than RSV-only mAbs (against RSV A and B) and similar to MPV-only mAbs (against MPV and MPV D280N).

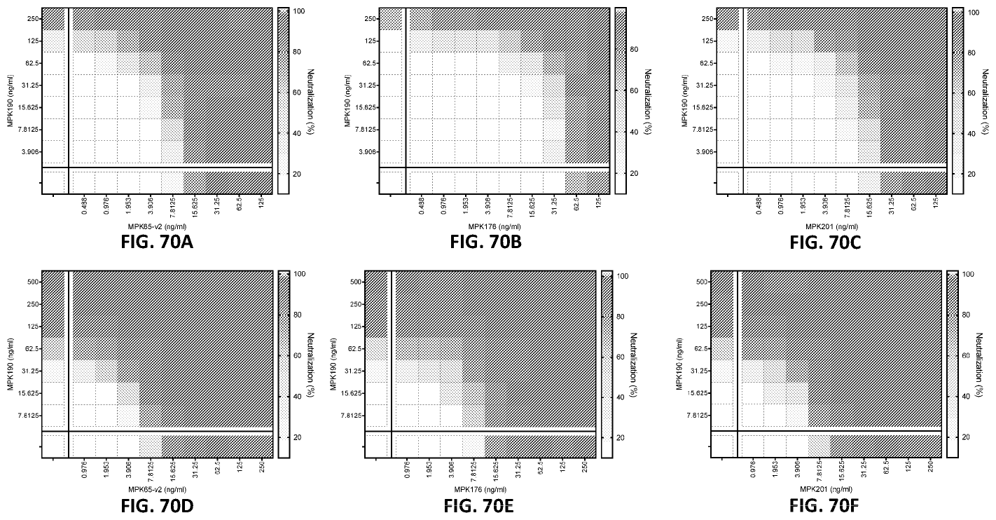

Synergism between MPK190 and MPK65-v2, MPK201, or MPK176 in both RSV A (FIGS. 70A-C) and RSV B (FIGS. 70D-F) neutralization was tested. Results are summarized Table 58. All three RSV-only antibodies shows synergism with MPK190 for RSV A and RSV B neutralization.

TABLE 58
Synergy scores of MPK190 and RSV-only antibodies

| | RSV A | RSV A | RSV B | RSV B | |
|---|---|---|---|---|---|
| | 95% | 99.9% | 95% | 99.9% | Antagonism |
| mAb | conf. | conf. | conf. | conf. | score |
| MPK65-v2 | 718.8 | 500.22 | 505.59 | 344.82 | 0 |
| MPK201 | 721.73 | 506.99 | 424.59 | 212.04 | 0 |
| MPK176 | 931.33 | 653.03 | 186.64 | 359.95 | 0 |

The pharmacokinetics of MPK130-v1.3-LS were investigated in cynomolgus monkeys injected with an IV dose of 5 mg/kg. Non-RSV/MPV comparator antibodies were also assessed. Results are presented in Table 59.

TABLE 59
PK Data for MPK190-v1.3-LS in Monkeys

| | MPK190- | | | | VIR- |
|---|---|---|---|---|---|
| | v1.3 | FNI9v8 | S2X324 | S2E12 | 7831 |
| T_{1/2} (days) | 15.2 | 20.8 | 22.8 | 25.2 | 16.1 |
| Cₘᵢₙ (ug/ml) | 5.39 | 8.33 | 22.1 | 13.6 | 4.59 |
| V_{z} (ml/kg) | 66.3 | 83.4 | 65.3 | 66.7 | 95.7 |
| Cl (ml/h/kg) | 0.129 | 0.118 | 0.0860 | 0.078 | 0.182 |
| AUCₗₐₛₜ | 1530 | 1530 | 2520 | 2760 | 1190 |
| (ug · d/ml) | | | | | |

Pharmacokinetics of MPK190-v1.3-LS (two batches) were investigated in SCID mice injected with an IV dose of 5 mg/kg. Non-RSV/MPV comparator antibodies were also assessed. Results are presented in Table 60.

TABLE 60
PK Data for MPK190-v1.3-LS in SCID Mice

| | HL_Lambda_{—} | Cₘₐₓ | Cₗₐₛₜ | AUCₗₐₛₜ | V_{z}_{_{—}}_{obs} | Cₗ_{_{—}}_{obs} |
|---|---|---|---|---|---|---|
| | z (day) | (ug/ml) | (ug/ml) | (day*ug/ml) | (ml/kg) | (ml/h/kg) |
| MPK190-v1.3 (Batch 1) | 15.9 | 87.3 | 2.23 | 735 | 163 | 0.295 |
| MPK190-v1.3 (Batch 2) | 15.5 | 114 | 2.62 | 882 | 131 | 0.246 |
| FNI9v8 | 16.6 | 82.2 | 2.10 | 738 | 151 | 0.265 |
| S2X324 | 15.8 | 105 | 2.78 | 914 | 116 | 0.214 |
| S2E12 | 18.5 | 103 | 1.60 | 685 | 183 | 0.290 |
| VIR-7831 | 14.0 | 86.1 | 1.18 | 671 | 148 | 0.306 |

Pharmacokinetics of MPK190-v1.3-LS and additional RSV-only antibodies was investigated in SCID mice injected with an IV injection at the indicated doses. Results are presented in Table 56.

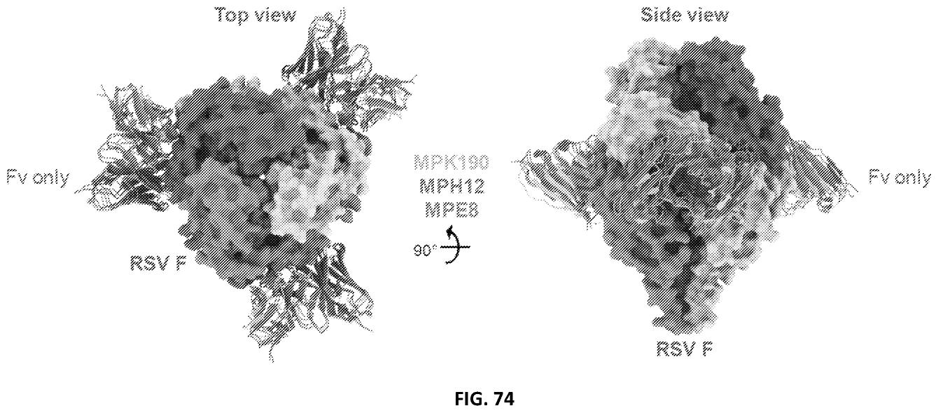

Predicted RSV F binding of MPK190-v1.3 to RSVF as compared to binding of comparator antibodies MPE8 and MPH12 was determined and is illustrated in FIG. 74.

Fab fragments of the full-length antibody were tested (FIG. 79A and FIG. 79B) and neutralization data was similar to that obtained using full-length antibody. Further results are presented in Table 61.

TABLE 61
Fab testing

| | RSV-long | RSV-B strain | RSV-B Strain | RSV-B1 (BEI |
|---|---|---|---|---|
| EC50 (nM) | strain | 18537 (ATCC) | 9320 (ATCC) | Bioresources) |
| MPK190-v.1.3- | 0.1048 | 0.0247 | 0.0170 | 0.0057 |
| rIgG1m, 17, 1-LS | | | | |
| MPK190-rFab | 0.0979 | 0.0633 | 0.0325 | 0.0172 |
| MED18897- | 0.0069 | 0.0110 | 0.0102 | 0.0088 |
| rIgG1-YTE | | | | |

Effector functions of various RSV-only antibodies are summarized in FIG. 80.

### Example 33

### Characterization of Mpk190-V1.3 and Mpk65-v2, Mpk176, and Mpk201 Antibody Combinations

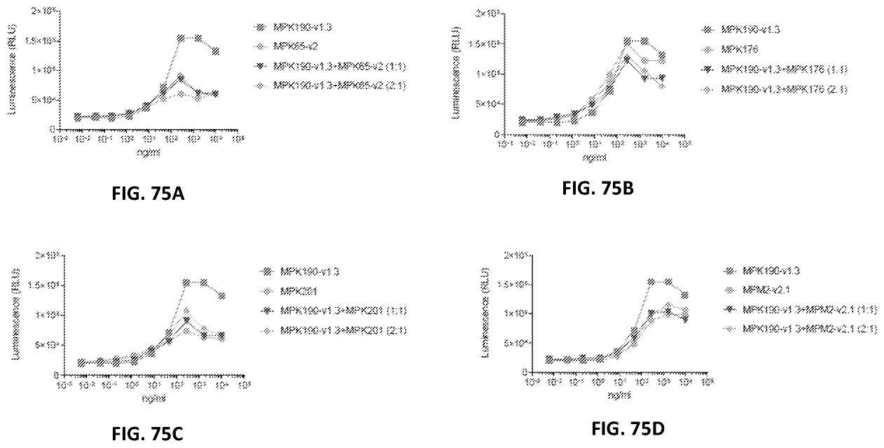

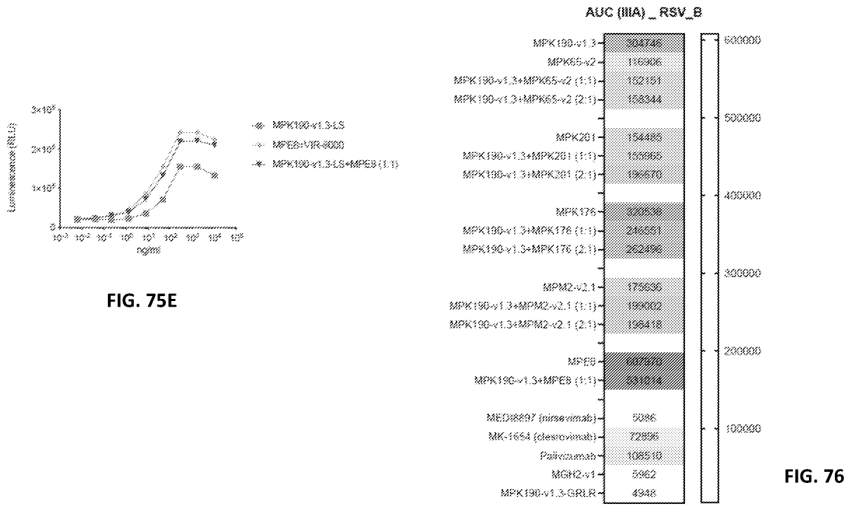

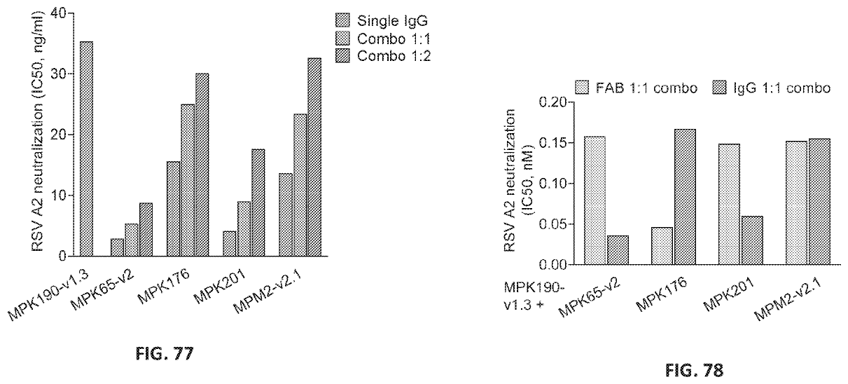

Combinations of MPK190-v1.3 and various RSV-only antibodies, specifically MPK64-v2, MPK65-v2, MPK176, and MPK201 were tested for additive effector function in Jurkat-FcgRIIIa (V158) Expi293 transfected with F protein of RSV B. Tests were otherwise performed as in other examples with these cells. Luminescence results after 22 hours are presented in FIGS. 75A-75E. AUC results are summarized in FIG. 76. The data from FIGS. 75A-75E is summarized in FIG. 77. Combinations of full-length antibodies and Fab fragments as compared to full-length antibodies are summarized in FIG. 78.

Combinations of MPK190-v1.3 and MPK65-v2, MPK176, and MPK201 showed additive neutralization as compared to MPK190-v1.3 alone.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 63/392,834, filed on Jul. 27, 2022, U.S. Patent Application No. 63/395,269, filed on Aug. 4, 2022, U.S. Patent Application No. 63/427,391, filed on Nov. 22, 2022, U.S. Patent Application No. 63/430,310, filed on Dec. 5, 2022, U.S. Patent Application No. 63/482,538, filed on Jan. 31, 2023, and U.S. Patent Application No. 63/494,751, filed on Apr. 6, 2023, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. An antibody or antigen-binding fragment comprising a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are according to the IMGT numbering system,
(A) wherein the antibody or antigen-binding fragment binds to a respiratory syncytial virus (RSV) fusion glycoprotein (RSV-F) and wherein: (i) the CDRH1 comprises the amino acid sequence set forth in any one of SEQ ID NOs.: 121, 3, 137, 147, 160, 170, 182, 216, 234, 244, 262, 278, 285, 301, 333, 363, 370, 728, 738, 747, 756, 766, 776, 785, 795, 805, 814, 887, 890, 13, 23, 31, 39, 54, 60, 74, 80, 87, 112, 130, 380, 387, 396, 417, 441, 452, 459, 471, 481, 492, 498, 505, 514, 528, and 533 or a functional variant thereof comprising one, two, or three acid substitutions, one or more of which substitutions is optionally a conservative substitution and/or is a substitution to a germline-encoded amino acid; (ii) the CDRH2 comprises the amino acid sequence set forth in any one of SEQ ID NOs.: 122, 4, 138, 161, 217, 226, 235, 263, 279, 302, 334, 364, 371, 729, 739, 748, 757, 767, 777, 786, 796, 815, 818, 838, 884, 894, 897, 14, 24, 32, 40, 47, 61, 81, 88, 96, 113, 388, 397, 405, 442, 453, 464, 506, 515, 534, 540, 543, and 548 or a functional variant thereof comprising one, two, or three amino acid substitutions, one or more of which substitutions is optionally a conservative substitution and/or is a substitution to a germline-encoded amino acid; (iii) the CDRH3 comprises the amino acid sequence set forth in any one of SEQ ID NOs.: 123, 5, 139, 148, 162, 176, 183, 190, 197, 203, 218, 227, 236, 245, 255, 264, 272, 280, 286, 294, 303, 310, 317, 322, 328, 335, 343, 353, 358, 365, 372, 730, 740, 749, 758, 768, 778, 787, 797, 806, 821, 824, 832, 835, 841, 844, 15, 25, 33, 48, 62, 89, 114, 131, 389, 398, 406, 427, 432, 476, 493, 499, 507, 516, 535, and 551 or a functional variant thereof comprising one, two, or three amino acid substitutions, one or more of which substitutions is optionally a conservative substitution and/or is a substitution to a germline-encoded amino acid; (iv) the CDRL1 comprises the amino acid sequence set forth in any one of SEQ ID NOs.: 18, 8, 142, 151, 165, 173, 186, 193, 206, 221, 230, 239, 258, 267, 289, 297, 306, 313, 338, 350, 375, 733, 743, 761, 771, 781, 790, 800, 809, 65, 92, 99, 126, 383, 401, 437, 445, 456, 488, 554, 561, 564, and 568 or a functional variant thereof comprising one, two, or three amino acid substitutions, one or more of which substitutions is optionally a conservative substitution and/or is a substitution to a germline-encoded amino acid; (v) the CDRL2 comprises the amino acid sequence set forth in any one of SEQ ID NOs.: 705, 9, 143, 152, 156, 166, 200, 207, 222, 240, 268, 290, 314, 339, 734, 752, 762, 772, 791, 801, 856, 871, 19, 43, 66, 117, 392, 409, 467, 489, 510, 519, and 555, or a functional variant thereof comprising one, two, or three amino acid substitutions, one or more of which substitutions is optionally a conservative substitution and/or is a substitution to a germline-encoded amino acid; and/or (vi) the CDRL3 comprises the amino acid sequence set forth in any one of SEQ ID NOs.: 127, 10, 144, 153, 157, 167, 179, 187, 194, 208, 213, 223, 231, 241, 248, 259, 269, 275, 291, 298, 307, 325, 340, 376, 735, 744, 753, 763, 773, 782, 792, 802, 810, 848, 852, 866, 20, 28, 36, 44, 51, 57, 67, 77, 84, 93, 118, 134, 384, 393, 402, 410, 420, 438, 446, 468, 502, 511, 556, and 520 or a functional variant thereof comprising having one, two, or three amino acid substitutions, one or more of which substitutions is optionally a conservative substitution and/or is a substitution to a germline-encoded amino acid;
(B) wherein the antibody or antigen-binding fragment binds to a metapneumovirus (MPV) fusion glycoprotein (MPV-F) and wherein: (i) the CDRH1 comprises the amino acid sequence set forth in any one of SEQ ID Nos.: 102, 13, 23, 31, 39, 54, 60, 74, 80, 87, 112, 121, 130, 380, 387, 396, 417, 441, 452, 459, 471, 481, 492, 498, 505, 514, 528, and 533 or a functional variant thereof comprising one, two, or three acid substitutions, one or more of which substitutions is optionally a conservative substitution and/or is a substitution to a germline-encoded amino acid; (ii) the CDRH2 comprises the amino acid sequence set forth in any one of SEQ ID Nos.: 103, 14, 24, 32, 40, 47, 61, 81, 88, 96, 113, 122, 388, 397, 405, 442, 453, 464, 506, 515, 534, 540, 543, and 548 or a functional variant thereof comprising one, two, or three amino acid substitutions, one or more of which substitutions is optionally a conservative substitution and/or is a substitution to a germline-encoded amino acid; (iii) the CDRH3 comprises the amino acid sequence set forth in any one of SEQ ID NO.: 104, 15, 25, 33, 48, 62, 89, 114, 123, 131, 389, 398, 406, 427, 432, 476, 493, 499, 507, 516, 535, and 551 or a functional variant thereof comprising one, two, or three amino acid substitutions, one or more of which substitutions is optionally a conservative substitution and/or is a substitution to a germline-encoded amino acid; (iv) the CDRL1 comprises the amino acid sequence set forth in any one of SEQ ID Nos.: 107, 18, 65, 92, 99, 126, 383, 401, 437, 445, 456, 488, 554, 561, 564, and 568 or a functional variant thereof comprising one, two, or three amino acid substitutions, one or more of which substitutions is optionally a conservative substitution and/or is a substitution to a germline-encoded amino acid; (v) the CDRL2 comprises the amino acid sequence set forth in any one of SEQ ID Nos.: 108, 19, 43, 66, 117, 392, 409, 467, 489, 510, 519, 555, and 705, or a functional variant thereof comprising one, two, or three amino acid substitutions, one or more of which substitutions is optionally a conservative substitution and/or is a substitution to a germline-encoded amino acid; and/or (vi) the CDRL3 comprises the amino acid sequence set forth in any one of SEQ ID Nos.: 109, 20, 28, 36, 44, 51, 57, 67, 77, 84, 93, 118, 127, 134, 384, 393, 402, 410, 420, 438, 446, 468, 502, 511, 556, and 520, or a functional variant thereof comprising having one, two, or three amino acid substitutions, one or more of which substitutions is optionally a conservative substitution and/or is a substitution to a germline-encoded amino acid; or
(C) wherein the antibody or antigen-binding fragment binds to a respiratory syncytial virus (RSV) fusion glycoprotein (RSV-F) and a metapneumovirus (MPV) fusion glycoprotein (MPV-F) and wherein: (i) the CDRH1 comprises the amino acid sequence set forth in any one of SEQ ID Nos.: 13, 23, 31, 39, 54, 60, 74, 80, 87, 112, 121, 130, 380, 387, 396, 417, 441, 452, 459, 471, 481, 492, 498, 505, 514, 528, and 533, or a functional variant thereof comprising one, two, or three acid substitutions, one or more of which substitutions is optionally a conservative substitution and/or is a substitution to a germline-encoded amino acid; (ii) the CDRH2 comprises the amino acid sequence set forth in any one of SEQ ID Nos.: 14, 24, 32, 40, 47, 61, 81, 88, 96, 113, 122, 388, 397, 405, 442, 453, 464, 506, 515, 534, 540, 543, and 548, or a functional variant thereof comprising one, two, or three amino acid substitutions, one or more of which substitutions is optionally a conservative substitution and/or is a substitution to a germline-encoded amino acid; (iii) the CDRH3 comprises the amino acid sequence set forth in any one of SEQ ID Nos.: 15, 25, 33, 48, 62, 89, 114, 123, 131, 389, 398, 406, 427, 432, 476, 493, 499, 507, 516, 535, and 551, or a functional variant thereof comprising one, two, or three amino acid substitutions, one or more of which substitutions is optionally a conservative substitution and/or is a substitution to a germline-encoded amino acid; (iv) the CDRL1 comprises the amino acid sequence set forth in any one of SEQ ID NOs.: 18, 65, 92, 99, 126, 383, 401, 437, 445, 456, 488, 554, 561, 564, and 568, or a functional variant thereof comprising one, two, or three amino acid substitutions, one or more of which substitutions is optionally a conservative substitution and/or is a substitution to a germline-encoded amino acid; (v) the CDRL2 comprises the amino acid sequence set forth in any one of SEQ ID Nos.: 19, 43, 66, 117, 392, 409, 467, 489, 510, 519, 555, and 705, or a functional variant thereof comprising one, two, or three amino acid substitutions, one or more of which substitutions is optionally a conservative substitution and/or is a substitution to a germline-encoded amino acid; and/or (vi) the CDRL3 comprises the amino acid sequence set forth in any one of SEQ ID Nos.: 20, 28, 36, 44, 51, 57, 67, 77, 84, 93, 118, 127, 134, 384, 393, 402, 410, 420, 438, 446, 468, 502, 511, 520, and 556, or a functional variant thereof comprising having one, two, or three amino acid substitutions, one or more of which substitutions is optionally a conservative substitution and/or is a substitution to a germline-encoded amino acid.

2. The antibody or antigen-binding fragment of claim 1, wherein:
(1) the VH and the VL comprise amino acid sequences having at least 90% identity to the amino acid sequences set forth in: (i) SEQ ID NOs.: 702 and 704, respectively; (ii) SEQ ID NOs.: 136 and 851, respectively; (iii) SEQ ID NOs.: 817 and 141, respectively; (iv) SEQ ID NOs.: 899 and 360, respectively; (v) SEQ ID NOs.: 136 and 851, respectively; (vi) SEQ ID NOs.: 817 and 141, respectively; (vii) SEQ ID NOs.: 233 and 858, respectively; (viii) SEQ ID NOs.: 837 and 858, respectively; (ix) SEQ ID NOs.: 357 and 847, respectively, wherein sequence variation is optionally limited to one or more framework regions and/or sequence variation comprises one or more substitution to a germline-encoded amino acid;
(2) the antibody or antigen-binding fragment comprises the CDRH1, CDRH2, CDRH3, CDRL1, CDRL2, and CDRL3 amino acid sequences of the VH and VL amino acid sequences set forth in (i) SEQ ID NOs.: 702 and 704, respectively; (ii) SEQ ID NOs.:136 and 851, respectively; (iii) SEQ ID NOs.: 817 and 141, respectively; (iv) SEQ ID NOs.: 899 and 360, respectively; (v) SEQ ID NOs.: 136 and 851, respectively; (vi) SEQ ID NOs.: 817 and 141, respectively; (vii) SEQ ID NOs.: 233 and 858, respectively; (viii) SEQ ID NOs.: 837 and 858, respectively; (ix) SEQ ID NOs.: 357 and 847, respectively; or
(3)(i) the CDRH1 comprises the amino acid sequence set forth in any one of SEQ ID NOs.: 121, 137, 814, 234, 814, or a functional variant thereof comprising one, two, or three acid substitutions, one or more of which substitutions is optionally a conservative substitution and/or is a substitution to a germline-encoded amino acid; (ii) the CDRH2 comprises the amino acid sequence set forth in any one of SEQ ID NOs.: 122, 138, 818, 235, 838, 226, or a functional variant thereof comprising one, two, or three amino acid substitutions, one or more of which substitutions is optionally a conservative substitution and/or is a substitution to a germline-encoded amino acid; (iii) the CDRH3 comprises the amino acid sequence set forth in any one of SEQ ID NOs.: 123, 139, 236, 358, or a functional variant thereof comprising one, two, or three amino acid substitutions, one or more of which substitutions is optionally a conservative substitution and/or is a substitution to a germline-encoded amino acid; (iv) the CDRL1 comprises the amino acid sequence set forth in any one of SEQ ID NOs.: 18, 142, 239, or a functional variant thereof comprising one, two, or three amino acid substitutions, one or more of which substitutions is optionally a conservative substitution and/or is a substitution to a germline-encoded amino acid; (v) the CDRL2 comprises the amino acid sequence set forth in any one of SEQ ID NOs.: 705, 143, 240, or a functional variant thereof comprising one, two, or three amino acid substitutions, one or more of which substitutions is optionally a conservative substitution and/or is a substitution to a germline-encoded amino acid; and/or (vi) the CDRL3 comprises the amino acid sequence set forth in any one of SEQ ID NOs.: 127, 852, 144, 241, 848, 231, or a functional variant thereof comprising having one, two, or three amino acid substitutions, one or more of which substitutions is optionally a conservative substitution and/or is a substitution to a germline-encoded amino acid.

3. The antibody or antigen-binding fragment of claim 1 or claim 2, wherein:
(A) the VH and the VL comprise amino acid sequences having at least 90% identity to the amino acid sequences set forth in: SEQ ID NOs.: 136 and 851, respectively, wherein sequence variation is optionally limited to one or more framework regions and/or sequence variation comprises one or more substitution to a germline-encoded amino acid;
(B) the antibody or antigen-binding fragment comprises the CDRH1, CDRH2, CDRH3, CDRL1, CDRL2, and CDRL3 amino acid sequences of the VH and VL amino acid sequences set forth in (i) SEQ ID NOs.: 136 and 851, respectively; or
(C)(i) the CDRH1 comprises the amino acid sequence set forth in SEQ ID NO.: 137, or a functional variant thereof comprising one, two, or three acid substitutions, one or more of which substitutions is optionally a conservative substitution and/or is a substitution to a germline-encoded amino acid; (ii) the CDRH2 comprises the amino acid sequence set forth in SEQ ID NO.: 138, or a functional variant thereof comprising one, two, or three amino acid substitutions, one or more of which substitutions is optionally a conservative substitution and/or is a substitution to a germline-encoded amino acid; (iii) the CDRH3 comprises the amino acid sequence set forth in SEQ ID NO.: 139, or a functional variant thereof comprising one, two, or three amino acid substitutions, one or more of which substitutions is optionally a conservative substitution and/or is a substitution to a germline-encoded amino acid; (iv) the CDRL1 comprises the amino acid sequence set forth in SEQ ID NO.: 142, or a functional variant thereof comprising one, two, or three amino acid substitutions, one or more of which substitutions is optionally a conservative substitution and/or is a substitution to a germline-encoded amino acid; (v) the CDRL2 comprises the amino acid sequence set forth in SEQ ID NO.: 143, or a functional variant thereof comprising one, two, or three amino acid substitutions, one or more of which substitutions is optionally a conservative substitution and/or is a substitution to a germline-encoded amino acid; and/or (vi) the CDRL3 comprises the amino acid sequence set forth in SEQ ID NO.: 852, or a functional variant thereof comprising having one, two, or three amino acid substitutions, one or more of which substitutions is optionally a conservative substitution and/or is a substitution to a germline-encoded amino acid.

4. The antibody or antigen-binding fragment of claim 1 or claim 2, wherein:
(A) the VH and the VL comprise amino acid sequences having at least 90% identity to the amino acid sequences set forth in: SEQ ID NOs.: 817 and 141, respectively, wherein sequence variation is optionally limited to one or more framework regions and/or sequence variation comprises one or more substitution to a germline-encoded amino acid;
(B) the antibody or antigen-binding fragment comprises the CDRH1, CDRH2, CDRH3, CDRL1, CDRL2, and CDRL3 amino acid sequences of the VH and VL amino acid sequences set forth in (i) SEQ ID NOs.: 817 and 141, respectively; or
(C)(i) the CDRH1 comprises the amino acid sequence set forth in SEQ ID NO.: 814, or a functional variant thereof comprising one, two, or three acid substitutions, one or more of which substitutions is optionally a conservative substitution and/or is a substitution to a germline-encoded amino acid; (ii) the CDRH2 comprises the amino acid sequence set forth in SEQ ID NO.: 818, or a functional variant thereof comprising one, two, or three amino acid substitutions, one or more of which substitutions is optionally a conservative substitution and/or is a substitution to a germline-encoded amino acid; (iii) the CDRH3 comprises the amino acid sequence set forth in SEQ ID NO.: 139, or a functional variant thereof comprising one, two, or three amino acid substitutions, one or more of which substitutions is optionally a conservative substitution and/or is a substitution to a germline-encoded amino acid; (iv) the CDRL1 comprises the amino acid sequence set forth in SEQ ID NO.: 142, or a functional variant thereof comprising one, two, or three amino acid substitutions, one or more of which substitutions is optionally a conservative substitution and/or is a substitution to a germline-encoded amino acid; (v) the CDRL2 comprises the amino acid sequence set forth in SEQ ID NO.: 143, or a functional variant thereof comprising one, two, or three amino acid substitutions, one or more of which substitutions is optionally a conservative substitution and/or is a substitution to a germline-encoded amino acid; and/or (vi) the CDRL3 comprises the amino acid sequence set forth in SEQ ID NO.: 144, or a functional variant thereof comprising having one, two, or three amino acid substitutions, one or more of which substitutions is optionally a conservative substitution and/or is a substitution to a germline-encoded amino acid.

5. The antibody or antigen-binding fragment of claim 1 or claim 2, wherein:
(A) the VH and the VL comprise amino acid sequences having at least 90% identity to the amino acid sequences set forth in: SEQ ID NOs.: 233 and 858, respectively, wherein sequence variation is optionally limited to one or more framework regions and/or sequence variation comprises one or more substitution to a germline-encoded amino acid;
(B) the antibody or antigen-binding fragment comprises the CDRH1, CDRH2, CDRH3, CDRL1, CDRL2, and CDRL3 amino acid sequences of the VH and VL amino acid sequences set forth in (i) SEQ ID NOs.: 233 and 858, respectively; of
(C)(i) the CDRH1 comprises the amino acid sequence set forth in SEQ ID NO.: 234, or a functional variant thereof comprising one, two, or three acid substitutions, one or more of which substitutions is optionally a conservative substitution and/or is a substitution to a germline-encoded amino acid; (ii) the CDRH2 comprises the amino acid sequence set forth in SEQ ID NO.: 235, or a functional variant thereof comprising one, two, or three amino acid substitutions, one or more of which substitutions is optionally a conservative substitution and/or is a substitution to a germline-encoded amino acid; (iii) the CDRH3 comprises of the amino acid sequence set forth in SEQ ID NO.: 236, or a functional variant thereof comprising one, two, or three amino acid substitutions, one or more of which substitutions is optionally a conservative substitution and/or is a substitution to a germline-encoded amino acid; (iv) the CDRL1 comprises the amino acid sequence set forth in SEQ ID NO.: 239, or a functional variant thereof comprising one, two, or three amino acid substitutions, one or more of which substitutions is optionally a conservative substitution and/or is a substitution to a germline-encoded amino acid; (v) the CDRL2 comprises the amino acid sequence set forth in SEQ ID NO.: 240, or a functional variant thereof comprising one, two, or three amino acid substitutions, one or more of which substitutions is optionally a conservative substitution and/or is a substitution to a germline-encoded amino acid; and/or (vi) the CDRL3 comprises the amino acid sequence set forth in SEQ ID NO.: 241, or a functional variant thereof comprising having one, two, or three amino acid substitutions, one or more of which substitutions is optionally a conservative substitution and/or is a substitution to a germline-encoded amino acid.

6. The antibody or antigen-binding fragment of claim 1 or claim 2, wherein:
(A) the VH and the VL comprise amino acid sequences having at least 90% identity to the amino acid sequences set forth in: SEQ ID NOs.: 837 and 858, respectively, wherein sequence variation is optionally limited to one or more framework regions and/or sequence variation comprises one or more substitution to a germline-encoded amino acid;
(B) the antibody or antigen-binding fragment comprises the CDRH1, CDRH2, CDRH3, CDRL1, CDRL2, and CDRL3 amino acid sequences of the VH and VL amino acid sequences set forth in (i) SEQ ID NOs.: 837 and 858, respectively;
(C)(i) the CDRH1 comprises o the amino acid sequence set forth in SEQ ID NO.: 234, or a functional variant thereof comprising one, two, or three acid substitutions, one or more of which substitutions is optionally a conservative substitution and/or is a substitution to a germline-encoded amino acid; (ii) the CDRH2 comprises the amino acid sequence set forth in SEQ ID NO.: 838, or a functional variant thereof comprising one, two, or three amino acid substitutions, one or more of which substitutions is optionally a conservative substitution and/or is a substitution to a germline-encoded amino acid; (iii) the CDRH3 comprises the amino acid sequence set forth in SEQ ID NO.: 236, or a functional variant thereof comprising one, two, or three amino acid substitutions, one or more of which substitutions is optionally a conservative substitution and/or is a substitution to a germline-encoded amino acid; (iv) the CDRL1 comprises the amino acid sequence set forth in SEQ ID NO.: 239, or a functional variant thereof comprising one, two, or three amino acid substitutions, one or more of which substitutions is optionally a conservative substitution and/or is a substitution to a germline-encoded amino acid; (v) the CDRL2 comprises the amino acid sequence set forth in SEQ ID NO.: 240, or a functional variant thereof comprising one, two, or three amino acid substitutions, one or more of which substitutions is optionally a conservative substitution and/or is a substitution to a germline-encoded amino acid; and/or (vi) the CDRL3 comprises the amino acid sequence set forth in SEQ ID NO.: 241, or a functional variant thereof comprising having one, two, or three amino acid substitutions, one or more of which substitutions is optionally a conservative substitution and/or is a substitution to a germline-encoded amino acid.

7. The antibody or antigen-binding fragment of claim 1 or claim 2, wherein:
(A) the VH and the VL comprise amino acid sequences having at least 90% identity to the amino acid sequences set forth in: SEQ ID NOs.: 357 and 847, respectively, wherein sequence variation is optionally limited to one or more framework regions and/or sequence variation comprises one or more substitution to a germline-encoded amino acid;
(B) the antibody or antigen-binding fragment comprises the CDRH1, CDRH2, CDRH3, CDRL1, CDRL2, and CDRL3 amino acid sequences of the VH and VL amino acid sequences set forth in (i) SEQ ID NOs.: 357 and 847, respectively;
(C)(i) the CDRH1 comprises the amino acid sequence set forth in SEQ ID NO.: 137, or a functional variant thereof comprising one, two, or three acid substitutions, one or more of which substitutions is optionally a conservative substitution and/or is a substitution to a germline-encoded amino acid; (ii) the CDRH2 comprises the amino acid sequence set forth in SEQ ID NO.: 226, or a functional variant thereof comprising one, two, or three amino acid substitutions, one or more of which substitutions is optionally a conservative substitution and/or is a substitution to a germline-encoded amino acid; (iii) the CDRH3 comprises the amino acid sequence set forth in SEQ ID NO.: 358, or a functional variant thereof comprising one, two, or three amino acid substitutions, one or more of which substitutions is optionally a conservative substitution and/or is a substitution to a germline-encoded amino acid; (iv) the CDRL1 comprises the amino acid sequence set forth in SEQ ID NO.: 142, or a functional variant thereof comprising one, two, or three amino acid substitutions, one or more of which substitutions is optionally a conservative substitution and/or is a substitution to a germline-encoded amino acid; (v) the CDRL2 comprises the amino acid sequence set forth in SEQ ID NO.: 143, or a functional variant thereof comprising one, two, or three amino acid substitutions, one or more of which substitutions is optionally a conservative substitution and/or is a substitution to a germline-encoded amino acid; and/or (vi) the CDRL3 comprises the amino acid sequence set forth in SEQ ID NO.: 848, or a functional variant thereof comprising having one, two, or three amino acid substitutions, one or more of which substitutions is optionally a conservative substitution and/or is a substitution to a germline-encoded amino acid.

8. The antibody or antigen-binding fragment of claim 1 or claim 2, wherein:
(A) the VH and the VL comprise amino acid sequences having at least 90% identity to the amino acid sequences set forth in: SEQ ID NOs.: 899 and 360, respectively, wherein sequence variation is optionally limited to one or more framework regions and/or sequence variation comprises one or more substitution to a germline-encoded amino acid;
(B) the antibody or antigen-binding fragment comprises the CDRH1, CDRH2, CDRH3, CDRL1, CDRL2, and CDRL3 amino acid sequences of the VH and VL amino acid sequences set forth in (i) SEQ ID NOs.: 899 and 360, respectively; or
(C)(i) the CDRH1 comprises the amino acid sequence set forth in SEQ ID NO.: 814, or a functional variant thereof comprising one, two, or three acid substitutions, one or more of which substitutions is optionally a conservative substitution and/or is a substitution to a germline-encoded amino acid; (ii) the CDRH2 comprises the amino acid sequence set forth in SEQ ID NO.: 226, or a functional variant thereof comprising one, two, or three amino acid substitutions, one or more of which substitutions is optionally a conservative substitution and/or is a substitution to a germline-encoded amino acid; (iii) the CDRH3 comprises the amino acid sequence set forth in SEQ ID NO.: 358, or a functional variant thereof comprising one, two, or three amino acid substitutions, one or more of which substitutions is optionally a conservative substitution and/or is a substitution to a germline-encoded amino acid; (iv) the CDRL1 comprises the amino acid sequence set forth in SEQ ID NO.: 142, or a functional variant thereof comprising one, two, or three amino acid substitutions, one or more of which substitutions is optionally a conservative substitution and/or is a substitution to a germline-encoded amino acid; (v) the CDRL2 comprises the amino acid sequence set forth in SEQ ID NO.: 143, or a functional variant thereof comprising one, two, or three amino acid substitutions, one or more of which substitutions is optionally a conservative substitution and/or is a substitution to a germline-encoded amino acid; and/or (vi) the CDRL3 comprises the amino acid sequence set forth in SEQ ID NO.: 231, or a functional variant thereof comprising having one, two, or three amino acid substitutions, one or more of which substitutions is optionally a conservative substitution and/or is a substitution to a germline-encoded amino acid.

9. The antibody or antigen-binding fragment of claim 1 or claim 2, wherein:
(A) the VH and the VL comprise amino acid sequences having at least 90% identity to the amino acid sequences set forth in: SEQ ID NOs.: 702 and 704, respectively, wherein sequence variation is optionally limited to one or more framework regions and/or sequence variation comprises one or more substitution to a germline-encoded amino acid;
(B) the antibody or antigen-binding fragment comprises the CDRH1, CDRH2, CDRH3, CDRL1, CDRL2, and CDRL3 amino acid sequences of the VH and VL amino acid sequences set forth in (i) SEQ ID NOs.: 702 and 704, respectively; or
(C)(i) the CDRH1 comprises the amino acid sequence set forth in SEQ ID NO.: 121, or a functional variant thereof comprising one, two, or three acid substitutions, one or more of which substitutions is optionally a conservative substitution and/or is a substitution to a germline-encoded amino acid; (ii) the CDRH2 comprises the amino acid sequence set forth in SEQ ID NO.: 122, or a functional variant thereof comprising one, two, or three amino acid substitutions, one or more of which substitutions is optionally a conservative substitution and/or is a substitution to a germline-encoded amino acid; (iii) the CDRH3 comprises the amino acid sequence set forth in SEQ ID NO.: 123, or a functional variant thereof comprising one, two, or three amino acid substitutions, one or more of which substitutions is optionally a conservative substitution and/or is a substitution to a germline-encoded amino acid; (iv) the CDRL1 comprises the amino acid sequence set forth in SEQ ID NO.: 18, or a functional variant thereof comprising one, two, or three amino acid substitutions, one or more of which substitutions is optionally a conservative substitution and/or is a substitution to a germline-encoded amino acid; (v) the CDRL2 comprises the amino acid sequence set forth in SEQ ID NO.: 705, or a functional variant thereof comprising one, two, or three amino acid substitutions, one or more of which substitutions is optionally a conservative substitution and/or is a substitution to a germline-encoded amino acid; and/or (vi) the CDRL3 comprises the amino acid sequence set forth in SEQ ID NO.: 127, or a functional variant thereof comprising having one, two, or three amino acid substitutions, one or more of which substitutions is optionally a conservative substitution and/or is a substitution to a germline-encoded amino acid.

10. The antibody or antigen-binding fragment of any one of claims 1-9, wherein the antibody or antigen-binding fragment is an IgG isotype selected from IgG1, IgG2, IgG3, and IgG4.

11. The antibody or antigen-binding fragment of any one of claims 1-10, wherein the antibody, or the antigen-binding fragment, comprises a human antibody, a monoclonal antibody, a purified antibody, a single chain antibody, a Fab, a Fab′, a F(ab′)2, or Fv.

12. The antibody or antigen-binding fragment of any one of claims 1-11, wherein the antibody or antigen-binding fragment is a multi-specific antibody or antigen-binding fragment, optionally a bispecific antibody or antigen-binding fragment, comprising (i) a first VH and a first VL; and (ii) a second VH and a second VL, wherein the first VH and the first VL together form a first antigen-binding site, and wherein the second VH and the second VL together form a second antigen-binding site; wherein the first VH and first VL, respectively, comprise: i) an amino acid sequence having at least 85% identity to the amino acid sequence set forth in any one of SEQ ID NOs.: 2, 136, 146, 159, 169, 175, 181, 189, 196, 202, 210, 215, 225, 233, 243, 250, 254, 261, 271, 277, 284, 293, 300, 309, 316, 321, 327, 332, 342, 347, 352, 357, 362, and 369; and/or an amino acid sequence having at least 85% identity to the amino acid sequence set forth in any one of SEQ ID NOs.: 7, 141, 150, 155, 164, 172, 178, 185, 192, 199, 205, 212, 220, 229, 238, 247, 252, 257, 266, 274, 282, 288, 296, 305, 312, 319, 324, 330, 337, 345, 349, 355, 360, 367, and 374; ii) an amino acid sequence having at least 85% identity to the amino acid sequence set forth in SEQ ID NO.: 101; and/or an amino acid sequence having at least 85% identity to the amino acid sequence set forth in SEQ ID NO.: 106; iii) an amino acid sequence having at least 85% identity to the amino acid sequence set forth in any one of SEQ ID NOs.: 129, 12, 38, 46, 53, 59, 69, 120, 73, 79, 86, 95, 22, 111, 30, 378, 386, 395, 404, 412, 416, 422, 426, 431, 440, 451, 458, 463, 470, 475, 480, 485, 491, 497, 532, 537, 539, 542, 545, 547, 550, 504, 513, 524, 527, 702, 707, 712, and 716; and/or an amino acid sequence having at least 85% identity to the amino acid sequence set forth in any one of SEQ ID NOs.: 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 553, 558, 560, 563, 567, 570, 572, 574, 704, and 710; iv) an amino acid sequence having at least 85% identity to the amino acid sequence set forth in any one of SEQ ID NOs.: 532, 537, 539, 542, 545, 547, and 550; and/or an amino acid sequence having at least 85% identity to the amino acid sequence set forth in any one of SEQ ID NOs.: 553, 558, 560, 563, 567, 570, 572, and 574; and/or v) an amino acid sequence having at least 85% identity to the amino acid sequence set forth in any one of SEQ ID NOs.: 2, 136, 146, 159, 169, 175, 181, 189, 196, 202, 210, 215, 225, 233, 243, 250, 254, 261, 271, 277, 284, 293, 300, 309, 316, 321, 327, 332, 342, 347, 352, 357, 362, 369, 369, 727, 737, 746, 755, 765, 775, 784, 794, 804, 813, 817, 820, 823, 826, 828, 831, 834, 837, 840, 843, 883, 886, 889, 893, 896, 899, 901, 903, 101, 129, 12, 38, 46, 53, 59, 69, 120, 73, 79, 86, 95, 22, 111, 30, 378, 386, 395, 404, 412, 416, 422, 426, 431, 440, 451, 458, 463, 470, 475, 480, 485, 491, 497, 504, 513, 524, 527, 702, 532, 537, 539, 542, 545, 547, 550, 707, 712, and 716; and/or an amino sequences having at least 85% identity to the amino acid sequence set forth in any one of SEQ ID NOs.: 7, 141, 150, 155, 164, 172, 178, 185, 192, 199, 205, 212, 220, 229, 238, 247, 252, 257, 266, 274, 282, 288, 296, 305, 312, 319, 324, 330, 337, 345, 349, 355, 360, 367, 374, 732, 742, 751, 760, 770, 780, 789, 799, 808, 847, 851, 855, 858, 860, 862, 865, 868, 870, 873, 875, 877, 879, 881, 106, 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 704, 553, 558, 560, 563, 567, 570, 572, 574, and 710, and wherein the second VH and second VL are not both the same as the first VH and first VH, and, respectively, comprise: i) an amino acid sequence having at least 85% identity to the amino acid sequence set forth in any one of SEQ ID NOs.: 2, 136, 146, 159, 169, 175, 181, 189, 196, 202, 210, 215, 225, 233, 243, 250, 254, 261, 271, 277, 284, 293, 300, 309, 316, 321, 327, 332, 342, 347, 352, 357, 362, 369, 727, 737, 746, 755, 765, 775, 784, 794, 804, 813, 817, 820, 823, 826, 828, 831, 834, 837, 840, 843, 883, 886, 889, 893, 896, 899, 901, and 903; and/or an amino acid sequence having at least 85% identity to the amino acid sequence set forth in any one of SEQ ID NOs.: 7, 141, 150, 155, 164, 172, 178, 185, 192, 199, 205, 212, 220, 229, 238, 247, 252, 257, 266, 274, 282, 288, 296, 305, 312, 319, 324, 330, 337, 345, 349, 355, 360, 367, 374 732, 742, 751, 760, 770, 780, 789, 799, 808, 847, 851, 855, 858, 860, 862, 865, 868, 870, 873, 875, 877, 879, and 881; ii) an amino acid sequence having at least 85% identity to the amino acid sequence set forth in SEQ ID NO.: 101; and/or an amino acid sequence having at least 85% identity to the amino acid sequence set forth in SEQ ID NO.: 106; iii) an amino acid sequence having at least 85% identity to the amino acid sequence set forth in any one of SEQ ID NOs.: 129, 12, 38, 46, 53, 59, 69, 120, 73, 79, 86, 95, 22, 111, 30, 378, 386, 395, 404, 412, 416, 422, 426, 431, 440, 451, 458, 463, 470, 475, 480, 485, 491, 497, 532, 537, 539, 542, 545, 547, 550, 504, 513, 524, 527, 702, 707, 712, and 716; and/or an amino acid sequence having at least 85% identity to the amino acid sequence set forth in any one of SEQ ID NOs.: 7, 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 553, 558, 560, 563, 567, 570, 572, 574, 704, and 710; iv) an amino acid sequence having at least 85% identity to the amino acid sequence set forth in any one of SEQ ID NOs.: 532, 537, 539, 542, 545, 547, and 550; and/or an amino acid sequence having at least 85% identity to the amino acid sequence set forth in any one of SEQ ID NOs.: 553, 558, 560, 563, 567, 570, 572, and 574; v) an amino acid sequence having at least 85% identity to the amino acid sequence set forth in any one of SEQ ID NOs.: 576, 586, 591, 600, 604, 613, and 617; and/or an amino acid sequence having at least 85% identity to the amino acid sequence set forth in any one of SEQ ID NOs.: 581, 588, 596, 602, 609, 615, and 622; vi) an amino acid sequence having at least 85% identity to the amino acid sequence set forth in any one of SEQ ID NOs.: 627, 636, 641, 648, 657, and 659; and/or an amino acid sequence having at least 85% identity to the amino acid sequence set forth in any one of SEQ ID NOs.: 632, 638, 645, 653, and 662; and/or vii) an amino acid sequence having at least 85% identity to the amino acid sequence set forth in any one of SEQ ID NOs.: 2, 136, 146, 159, 169, 175, 181, 189, 196, 202, 210, 215, 225, 233, 243, 250, 254, 261, 271, 277, 284, 293, 300, 309, 316, 321, 327, 332, 342, 347, 352, 357, 362, 369, 727, 737, 746, 755, 765, 775, 784, 794, 804, 813, 817, 820, 823, 826, 828, 831, 834, 837, 840, 843, 883, 886, 889, 893, 896, 899, 901, 903, 101, 129, 12, 38, 46, 53, 59, 69, 120, 73, 79, 86, 95, 22, 111, 30, 378, 386, 395, 404, 412, 416, 422, 426, 431, 440, 451, 458, 463, 470, 475, 480, 485, 491, 497, 504, 513, 524, 527, 702, 532, 537, 539, 542, 545, 547, 550, 576, 586, 591, 600, 604, 613, 617, 627, 636, 641, 648, 657, 659, 707, 712, and 716; and/or an amino sequences having at least 85% identity to the amino acid sequence set forth in any one of SEQ ID NOs.: 7, 141, 150, 155, 164, 172, 178, 185, 192, 199, 205, 212, 220, 229, 238, 247, 252, 257, 266, 274, 282, 288, 296, 305, 312, 319, 324, 330, 337, 345, 349, 355, 360, 367, 374, 732, 742, 751, 760, 770, 780, 789, 799, 808, 847, 851, 855, 858, 860, 862, 865, 868, 870, 873, 875, 877, 879, 881, 106, 133, 17, 42, 50, 56, 64, 71, 125, 76, 83, 91, 98, 27, 116, 35, 382, 391, 400, 408, 414, 419, 424, 429, 434, 436, 444, 449, 455, 461, 466, 473, 478, 483, 487, 495, 501, 509, 518, 522, 530, 704, 553, 558, 560, 563, 567, 570, 572, 574, 581, 588, 596, 602, 609, 615, 622, 632, 638, 645, 653, 662, and 710.

13. The antibody or antigen-binding fragment of claim 12, comprising:
(A)(i) a first VH and a first VL; and (ii) a second VH and a second VL, wherein the first VH and the first VL together form a first antigen-binding site, and wherein the second VH and the second VL together form a second antigen-binding site; wherein the first VH and first VL, respectively, comprise the VH and a VL set forth in SEQ ID NOs.: 136 and 851; 814 and 141; 233 and 858; 837 and 858; 357 and 847; and 899 and 360, respectively;
(B)(i) a first VH and a first VL; and (ii) a second VH and a second VL, wherein the first VH and the first VL together form a first antigen-binding site, and wherein the second VH and the second VL together form a second antigen-binding site; wherein the first VH and first VL, respectively, comprise the VH and VL as set forth in SEQ ID NOs.: 702 and 704; or
(C)(i) a first VH and a first VL; and (ii) a second VH and a second VL, wherein the first VH and the first VL together form a first antigen-binding site, and wherein the second VH and the second VL together form a second antigen-binding site; wherein the first VH and VL comprise a VH and a VL set forth in SEQ ID NOs.: 136 and 851; 814 and 141; 233 and 858; 837 and 858; 357 and 847; and 899 and 360, respectively; and the second VH and VL comprise a VH and VL as set forth in SEQ ID NOs.: 702 and 704.

14. The antibody or antigen-binding fragment of any one of claims 1-13, wherein the antibody or antigen-binding fragment comprises a Fc polypeptide or a fragment thereof.

15. The antibody or antigen-binding fragment of claim 14, wherein the Fc polypeptide comprises a Fc polypeptide or fragment thereof that comprises an amino acid sequence having at least 85% identity to any one of SEQ ID NOs.: 679-684 and 688-690, optionally other than naturally occurring variants thereof, or that comprises an amino acid sequence set forth in any one of SEQ ID NOs.: 679-684 and 688-690.

16. The antibody or antigen-binding fragment of claim 15, wherein the antibody comprises a heavy chain (HC) that comprises a polypeptide or fragment thereof that comprises the amino acid sequence set forth in SEQ ID NO.: 723, and a light chain (LC) that comprises a polypeptide or fragment thereof that comprises the amino acid sequence set forth in SEQ ID NO.: 725.

17. The antibody or antigen-binding fragment of any one of claims 1-16, wherein the antibody or antigen-binding fragment thereof binds to a) both a RSV A and RSV B strain; b) both a MPV A and MPV B strain; c) any combinations of an RSV A, RSV B, MPV A, and MPV B strain.

18. The antibody or antigen-binding fragment of any one of claims 1-17, wherein the antibody or antigen-binding fragment thereof (A) activates a human FcγRIIa or (B) activates a human FcγRIIIa.

19. The antibody or antigen-binding fragment of any one of claims 1-18, wherein (A) the antibody neutralizes infection by a RSV and/or a MPV; and/or (B) the antibody or antigen-binding fragment treats and/or prevents (i) a RSV infection and/or (ii) a MPV infection in a subject.

20. An isolated polynucleotide encoding the antibody or antigen-binding fragment of any one of claims 1-19, or encoding a VH, a heavy chain, a VL, a light chain and/or one or more CDR of the antibody or the antigen-binding fragment.

21. The isolated polynucleotide of claim 20, wherein the polynucleotide comprises a polynucleotide having at least 50% identity to the VH-encoding polynucleotide sequence set forth in any one or more of SEQ ID NOs.: 701, 135, 816, 232, 836, 356, 898, and/or the VL-encoding polynucleotide sequence set forth in any one or more of SEQ ID NOs.: 703, 850, 140, 857, 846, 359.

22. A recombinant vector comprising the polynucleotide of claim 20 or claim 21.

23. A host cell comprising the polynucleotide of claim 20 or claim 21 and/or the vector of claim 22, wherein the polynucleotide is heterologous to the host cell and wherein the host cell expresses the encoded antibody or antigen-binding fragment.

24. An isolated human B cell comprising the polynucleotide of claim 20 or claim 21 and/or the vector of claim 22, wherein polynucleotide is heterologous to the human B cell and/or wherein the human B cell is immortalized.

25. A composition comprising: (i) the antibody or antigen-binding fragment of any one of claims 1-19; (ii) the polynucleotide of claim 20 or claim 21; (iii) the recombinant vector of claim 22; (iv) the host cell of claim 23; and/or (v) the human B cell of claim 24, and a pharmaceutically acceptable excipient, carrier, or diluent.

26. The composition of claim 25, comprising a first antibody or antigen-binding fragment and a second antibody or antigen-binding fragment, wherein each of the first antibody or antigen-binding fragment and the second antibody or antigen-binding fragment are different and are each according any one of claims 1-19, or at least one is according to any one of claims 1-19 and at least one is palivizumab, nirsevimab or clesrovimab, or and antibody having a VH and VL of any one of palivizumab, nirsevimab or clesrovimab.

27. The composition of claim 25 or claim 26, comprising a first antibody or antigen-binding fragment and a second antibody or antigen-binding fragment, wherein:
(A) the first antibody or antigen-binding fragment comprises at least a VH and VL or CDRs of the VH and a VL set forth in any one of SEQ ID NOs.: 136 and 851; 814 and 141; 233 and 858; 837 and 858; 357 and 847; and 899 and 360, respectively;
(B) the second antibody or antigen-binding fragment comprises at least a VH and VL or CDRs of the VH and VL as set forth in SEQ ID NOs.: 702 and 704; or
(C) the first antibody or antigen-binding fragment comprises the VH and a VL set forth in anyone of SEQ ID NOs.: 136 and 851; 814 and 141; 233 and 858; 837 and 858; 357 and 847; and 899 and 360, respectively; and the second antibody or antigen-bindgin fragment comprises a second VH and VL or second CDRs of the VH and VL as set forth in SEQ ID NOs.: 702 and 704.

28. A composition comprising the polynucleotide of claim 20 or claim 21 or the vector of claim 22 encapsulated in a carrier molecule, wherein the carrier molecule optionally comprises a lipid, a lipid-derived delivery vehicle, such as a liposome, a solid lipid nanoparticle, an oily suspension, a submicron lipid emulsion, a lipid microbubble, an inverse lipid micelle, a cochlear liposome, a lipid microtubule, a lipid microcylinder, lipid nanoparticle (LNP), or a nanoscale platform.

29. A method of making an antibody or antigen-binding fragment of any one of claims 1-19, comprising culturing the host cell of embodiment claim 23 or the human B cell of claim 24 for a time and under conditions sufficient for the host cell or human B cell, respectively, to express the antibody or antigen-binding fragment.

30. A method of treating and/or preventing a RSV infection and/or a MPV infection in a subject, the method comprising administering to the subject an effective amount of: (i) the antibody or antigen-binding fragment of any one of claims 1-19; (ii) the polynucleotide of claim 20 or claim 21; (iii) the recombinant vector of claim 22; (iv) the host cell of claim 23; (v) the human B cell of claim 24; and/or (vi) the composition of any one of claims 25-28.

31. The antibody or antigen-binding fragment of any one of claims 1-19, the polynucleotide of claim 20 or claim 21, the recombinant vector of claim 22, the host cell of claim 23, the human B cell of claim 24, and/or the composition of any one of claims 25-28, for use:
(A) in a method of treating or preventing a RSV infection and/or a MPV infection in a subject; or
(B) in the preparation of a medicament for the treatment or prevention of a RSV infection and/or a MPV infection in a subject.

32. The method of claim 30 or the antibody or antigen-binding fragment, the polynucleotide, the recombinant vector, the host cell, the human B cell, and/or the composition for the use of claim 31, wherein: a) the RSV comprises both a RSV A and RSV B strain; b) the MPV comprises both a MPV A and MPV B strain; c) the RSV and MPV comprise any combinations of a RSV A, RSV B, MPV A, and MPV B strain.

33. A method for in vitro diagnosis of a RSV infection and/or a MPV infection, the method comprising: (i) contacting a sample from a subject with an antibody or antigen-binding fragment of any one of claims 1-19; and (ii) detecting a complex comprising an antigen and the antibody, or comprising an antigen and the antigen-binding fragment.

34. A kit comprising a liquid composition comprising and antibody or antigen-binding fragment of any one of claims 1-19, a polynucleotide according to claim 20 or claim 21, a recombinant vector according to claim 22, a host cell of any one of claim 23 or 24, or a composition of any one of claims 25-28 and instructions for use thereof in treating a RSV and/or MPV infection in a subject.


SEQUENCE LISTING (905 sequences)

SEQ ID NO: 1 - DNA, 376 bp, synthetic construct
  source 1..376: mol_type = other DNA; note = MPK44 VH
caggtccagctggtgcagtctcgggctgaggtgaagaagcctggggcctcagtgaaggtctcctgcaaggttctcggagacggcctcactgaactagccatgcactgggtgcgacaggctcctggaaaagggcttgagtggatgggaggttttcatcgtgaagggggtgagacagtctatgcacagaagtttcagggcagagtcaccatgaccgaggacacatattcgaacacagcctacatggagttgagcagcctgcgatctgccgacacggccatgtattattgtacaacattaatggacatagcggtgacgaccgccggagcaggagccaaccactactggggccagggaaccatggtcaccgtctcctcag

SEQ ID NO: 2 - protein, 125 aa, synthetic construct
  source 1..125: note = MPK44 VH
QVQLVQSRAEVKKPGASVKVSCKVLGDGLTELAMHWVRQAPGKGLEWMGGFHREGGETVYAQKFQGRVTMTEDTYSNTAYMELSSLRSADTAMYYCTTLMDIAVTTAGAGANHYWGQGTMVTVSS

SEQ ID NO: 3 - protein, 8 aa, synthetic construct
  source 1..8: note = MPK44 CDR-H1 (aa)
GDGLTELA

SEQ ID NO: 4 - protein, 8 aa, synthetic construct
  source 1..8: note = MPK44 CDR-H2 (aa)
FHREGGET

SEQ ID NO: 5 - protein, 18 aa, synthetic construct
  source 1..18: note = MPK44 CDR-H3 (aa)
TTLMDIAVTTAGAGANHY

SEQ ID NO: 6 - DNA, 337 bp, synthetic construct
  source 1..337: mol_type = other DNA; note = MPK44 VL (nt)
gatattgtgatgactcagtctccactctccctgcccgtcacccctggagagccggcctccatctcctgcaggtctagtcagagcctcctgcatagtaatggatacaactatttggattggtacctgcagaagccagggcagtctccacaggtcctgatctttttgggttctaatcgggcctccggggtccctgacaggtttagtggcagtggatccggcacagatttcacactgaaaatcaccagagtggaggctgaggatgttggggtgtatttctgcatgcaaggtctacagactcccttcactttcggcggagggaccagggtggatatcaaac

SEQ ID NO: 7 - protein, 112 aa, synthetic construct
  source 1..112: note = MPK44 VL (aa)
DIVMTQSPLSLPVTPGEPASISCRSSQSLLHSNGYNYLDWYLQKPGQSPQVLIFLGSNRASGVPDRFSGSGSGTDFTLKITRVEAEDVGVYFCMQGLQTPFTFGGGTRVDIK

SEQ ID NO: 8 - protein, 11 aa, synthetic construct
  source 1..11: note = MPK44; MPK202 CDR-L1 (aa)
QSLLHSNGYNY

SEQ ID NO: 9 - skipped

SEQ ID NO: 10 - protein, 9 aa, synthetic construct
  source 1..9: note = MPK44; MPK202 CDR-L3 (aa)
MQGLQTPFT

SEQ ID NO: 11 - DNA, 361 bp, synthetic construct
  source 1..361: mol_type = other DNA; note = MPK10 VH (nt)
gaggtgcagctggtgcagtctgggggaggcttggtcaagcctggagggtccctgaggctctcctgtgcagcctctggattcaggttcagtggttactttatgatctggatccgtcaggcgccaggtaaggggctggagtggatttctttcatcagtggaggaagttcctacacaaattacgctgactctgtgaagggccgattcaccatttccagagacaacgtccagaacacactctttctcgacatgaatagcctgagagccgaagatacgggtgtctatttctgtgcgacagaacaggtgccgacacctggcccctttgacctctggggccagggaagcctggtcaccgtctcctcag

SEQ ID NO: 12 - protein, 120 aa, synthetic construct
  source 1..120: note = MPK10 VH (aa)
EVQLVQSGGGLVKPGGSLRLSCAASGFRFSGYFMIWIRQAPGKGLEWISFISGGSSYTNYADSVKGRFTISRDNVQNTLFLDMNSLRAEDTGVYFCATEQVPTPGPFDLWGQGSLVTVSS

SEQ ID NO: 13 - protein, 8 aa, synthetic construct
  source 1..8: note = MPK10 CDR-H1 (aa)
GFRFSGYF

SEQ ID NO: 14 - protein, 8 aa, synthetic construct
  source 1..8: note = MPK10 CDR-H2 (aa)
ISGGSSYT

SEQ ID NO: 15 - protein, 13 aa, synthetic construct
  source 1..13: note = MPK10 CDR-H3 (aa)
ATEQVPTPGPFDL

SEQ ID NO: 16 - DNA, 334 bp, synthetic construct
  source 1..334: mol_type = other DNA; note = MPK10 VL (nt)
cagtctgtgctgacgcagccgccctcggtgtctggggtcccagggcagagggtcatcatctcctgcactgggagcagctccaacatcggggcaggttatgatgtacactggtatcaacagtttccaggaagatcccccacactcctcatctatcgtaataccaatcggccctcaggggtccctgaccgattctctggcttccagtccggctcctcagcctccctggccatcactggactccaggctgaggatgaggctgattattactgccagtcctttgaccgaaccctggatggatgggctttcggcggagggaccaaggtgaccgtcctag

SEQ ID NO: 17 - protein, 111 aa, synthetic construct
  source 1..111: note = MPK10 VL (aa)
QSVLTQPPSVSGVPGQRVIISCTGSSSNIGAGYDVHWYQQFPGRSPTLLIYRNTNRPSGVPDRFSGFQSGSSASLAITGLQAEDEADYYCQSFDRTLDGWAFGGGTKVTVL

SEQ ID NO: 18 - protein, 9 aa, synthetic construct
  source 1..9: note = MPK10; MPK9; MPK18; MPK30-v1; MPK36-v3; MPK67; MPK77; MPK77-v1.1; MPK86; MPK102; MPK104; MPK104-v1.1; MPK104-v1.3; MPK108; MPK127; MPK130; MPK133; MPK142-v1; MPK145; MPK149 CDR-L1 (aa)
SSNIGAGYD

SEQ ID NO: 19 - skipped

SEQ ID NO: 20 - protein, 11 aa, synthetic construct
  source 1..11: note = MPK10 CDR-L3 (aa)
QSFDRTLDGWA

SEQ ID NO: 21 - DNA, 361 bp, synthetic construct
  source 1..361: mol_type = other DNA; note = MPK102 VH (nt)
gaggtgcagctggtggagtctgggggaggcttggtcaagcctagagggtccctaagactctcctgtgcagcctctggattcaccttcagtggctacttcatgatctggatccgccaggccccaggtaaggggctggagtgggtctcatacatcagtggcagtagtacttacacaaactacgctgactctgtgaagggccgattcaccatctccagagacaacggccggaacacactttatctccaaatgaacagcctgagagccgacgacacggctgtctattattgtgcgacagaacaaacgccgtcacctggccccttcgacctctggggccagggaaccctggtcaccgtctcctcag

SEQ ID NO: 22 - protein, 120 aa, synthetic construct
  source 1..120: note = MPK102 VH (aa)
EVQLVESGGGLVKPRGSLRLSCAASGFTFSGYFMIWIRQAPGKGLEWVSYISGSSTYTNYADSVKGRFTISRDNGRNTLYLQMNSLRADDTAVYYCATEQTPSPGPFDLWGQGTLVTVSS

SEQ ID NO: 23 - protein, 8 aa, synthetic construct
  source 1..8: note = MPK102 CDR-H1 (aa)
GFTFSGYF

SEQ ID NO: 24 - protein, 8 aa, synthetic construct
  source 1..8: note = MPK102; MPK36-v3; MPK77; MPK77-v1.1; MPK133; MPK149 CDR-H2 (aa)
ISGSSTYT

SEQ ID NO: 25 - protein, 13 aa, synthetic construct
  source 1..13: note = MPK102; MPK18; MPK36-v3; MPK67; MPK86; MPK126; MPK204; MPK155; MPK151; MPK149 CDR-H3 (aa)
ATEQTPSPGPFDL

SEQ ID NO: 26 - DNA, 334 bp, synthetic construct
  source 1..334: mol_type = other DNA; note = MPK102 VL (nt)
cagtctgtgctgacgcagccgccctcagtgtctggggccccagggcagagggtcaccatctcctgcactgggaccagctccaacatcggggcaggttatgatgtgcactggtaccaacagtttccaggaacatcccccaaactcctcatctttcgtaataccaatcggccctcaggggtctctgaccgattctctggctccaagtctggctcctcagcctccctggccatcgctgggctccaggctgaggatgagtctgattattactgccagtcctatgacagaggcctggatggatgggttttcggcggagggaccaggctgaccgtcctag

SEQ ID NO: 27 - protein, 111 aa, synthetic construct
  source 1..111: note = MPK102 VL (aa)
QSVLTQPPSVSGAPGQRVTISCTGTSSNIGAGYDVHWYQQFPGTSPKLLIFRNTNRPSGVSDRFSGSKSGSSASLAIAGLQAEDESDYYCQSYDRGLDGWVFGGGTRLTVL

SEQ ID NO: 28 - protein, 11 aa, synthetic construct
  source 1..11: note = MPK102; MPK141 CDR-L3 (aa)
QSYDRGLDGWV

SEQ ID NO: 29 - DNA, 361 bp, synthetic construct
  source 1..361: mol_type = other DNA; note = MPK108 VH (nt)
gaggtgcagctggtggagtcggggggaggcttggtcaggcctggacagtccctgagactctcctgtacagcctctggattcagcatcagtggctacttcatgatctgggtccgccaggctccagggaaggggctggagtggatttcatacgttagcgccagtagcacatacacaaattacgcggactctgtgaggggccgattcaccacttccagagacaacggccagaactcactgtttctgcaaatggacagtctgacagccgaggacacgggtgtgtacttctgtgcgcgagaacagacaccgtcacctggcccctttgactcctggggccagggagtcctggtcaccgtctcctcag

SEQ ID NO: 30 - protein, 120 aa, synthetic construct
  source 1..120: note = MPK108 VH (aa)
EVQLVESGGGLVRPGQSLRLSCTASGFSISGYFMIWVRQAPGKGLEWISYVSASSTYTNYADSVRGRFTTSRDNGQNSLFLQMDSLTAEDTGVYFCAREQTPSPGPFDSWGQGVLVTVSS

SEQ ID NO: 31 - protein, 8 aa, synthetic construct
  source 1..8: note = MPK108 CDR-H1 (aa)
GFSISGYF

SEQ ID NO: 32 - protein, 8 aa, synthetic construct
  source 1..8: note = MPK108 CDR-H2 (aa)
VSASSTYT

SEQ ID NO: 33 - protein, 13 aa, synthetic construct
  source 1..13: note = MPK108 CDR-H3 (aa)
AREQTPSPGPFDS

SEQ ID NO: 34 - DNA, 334 bp, synthetic construct
  source 1..334: mol_type = other DNA; note = MPK108 VL (nt)
cagtctgtgctgacgcagccgccctcactgtctggggccccagggcagaggatcaccatctcctgcactgggaccagctccaacatcggggcaggttatgatgtccactggtaccagtaccttccaggaacagcccccaaactcgtcatctatcgtaacaccaatcggccctcaggggtctctggccggttctctggctccaagtctggcacgtcagcctcactggccatcactggcctccagactgaggatgaggctgattattactgccagtcctatgacaaaaaattggatggttgggtgttcggcggagggaccaagctgaccgtcctag

SEQ ID NO: 35 - protein, 111 aa, synthetic construct
  source 1..111: note = MPK108 VL (aa)
QSVLTQPPSLSGAPGQRITISCTGTSSNIGAGYDVHWYQYLPGTAPKLVIYRNTNRPSGVSGRFSGSKSGTSASLAITGLQTEDEADYYCQSYDKKLDGWVFGGGTKLTVL

SEQ ID NO: 36 - protein, 11 aa, synthetic construct
  source 1..11: note = MPK108 CDR-L3 (aa)
QSYDKKLDGWV

SEQ ID NO: 37 - DNA, 361 bp, synthetic construct
  source 1..361: mol_type = other DNA; note = MPK18 VH (nt)
gaggtgcagctggtggagtctgggggaggcttggtcaagcctggagggtccctaagactctcctgtgcagcctctggattcaccatcagtggctactacatgatctggatccgccaggctccagggaaggggctggagtggatttcatacataagtggcagtagttcttatacaaacttcggtgcctctgtgaagggccgattcaccatctccagggacaacggccaaaacacagtgcatctccaaatgaccaacctgagagccgacgacacggccctctattattgtgcgacagaacagacgccgtcacctggccccttcgacctctggggccagggaaccctggtcaccgtctcctcag

SEQ ID NO: 38 - protein, 120 aa, synthetic construct
  source 1..120: note = MPK18 VH (aa)
EVQLVESGGGLVKPGGSLRLSCAASGFTISGYYMIWIRQAPGKGLEWISYISGSSSYTNFGASVKGRFTISRDNGQNTVHLQMTNLRADDTALYYCATEQTPSPGPFDLWGQGTLVTVSS

SEQ ID NO: 39 - protein, 8 aa, synthetic construct
  source 1..8: note = MPK18; MPK30-v1; MPK67; MPK99; MPK141 CDR-H1 (aa)
GFTISGYY

SEQ ID NO: 40 - protein, 8 aa, synthetic construct
  source 1..8: note = MPK18; MPK67; MPK141; MPK152 CDR-H2 (aa)
ISGSSSYT

SEQ ID NO: 41 - DNA, 334 bp, synthetic construct
  source 1..334: mol_type = other DNA; note = MPK18 VL (nt)
cagtctgtcgtgacgcagccgccctcagtgtctggggccccaggacagagggtcaccatctcctgctctgggaccagctccaacatcggggcaggttatgatgtacactggtaccaacaatttccagggacatcccccaaactcctcatctctcgtaatttcaatcggccctcaggagtccctgcccgattctctggctccaagtctggctcctcagcctccctggccatcgctggactccaggctgaggatgaggctgattattactgccagtcctatgacagaagcctgggtggatgggttttcggcggagggaccaaggtgaccgtcctag

SEQ ID NO: 42 - protein, 111 aa, synthetic construct
  source 1..111: note = MPK18 VL (aa)
QSVVTQPPSVSGAPGQRVTISCSGTSSNIGAGYDVHWYQQFPGTSPKLLISRNFNRPSGVPARFSGSKSGSSASLAIAGLQAEDEADYYCQSYDRSLGGWVFGGGTKVTVL

SEQ ID NO: 43 - skipped

SEQ ID NO: 44 - protein, 11 aa, synthetic construct
  source 1..11: note = MPK18; MPK67; MPK99 CDR-L3 (aa)
QSYDRSLGGWV

SEQ ID NO: 45 - DNA, 361 bp, synthetic construct
  source 1..361: mol_type = other DNA; note = MPK30-v1 VH (nt)
gaggtgcagctggtggagtctgggggaggcttggtcaagcctggagggtccctaagactctcctgtgcagcctctggcttcaccatcagtggctactacatgatctggatccgccaggctccagggaaggggctggagtggatttcattcattagcgccagtagttcttacataaactacgctgactctgtgaggggccgattcaccatctccagggacaacggccggagcacagtatatctccaaatgaacagcctgagacccgaggatacggctgtctattattgtgcgacagaacaagtgccgtcacctggccccttcgacctctggggccagggaaccctggtcaccgtctcctcag

SEQ ID NO: 46 - protein, 120 aa, synthetic construct
  source 1..120: note = MPK30-v1 VH (aa)
EVQLVESGGGLVKPGGSLRLSCAASGFTISGYYMIWIRQAPGKGLEWISFISASSSYINYADSVRGRFTISRDNGRSTVYLQMNSLRPEDTAVYYCATEQVPSPGPFDLWGQGTLVTVSS

SEQ ID NO: 47 - protein, 8 aa, synthetic construct
  source 1..8: note = MPK30-v1; MPK9; MPK157; MPK158; MPK145; MPK142-v2; MPK142-v1 CDR-H2 (aa)
ISASSSYI

SEQ ID NO: 48 - protein, 13 aa, synthetic construct
  source 1..13: note = MPK30-v1; MPK77; MPK77-v1.1; MPK99; MPK133; MPK144; MPK146; MPK153 CDR-H3 (aa)
ATEQVPSPGPFDL

SEQ ID NO: 49 - DNA, 334 bp, synthetic construct
  source 1..334: mol_type = other DNA; note = MPK30-v1 VL (nt)
cagtctgtgttgacgcagccgccctcagtgtccggggccccagggcagagggtcaccatctcctgcactgggaccagctccaacatcggggcaggttatgatgtacactggtaccaacagtttccaggaagatcccccaaactcctcatctatcgtaataccaatcggccctcaggggtccctgaccgattctctggctccaagtctggctcctcagcctccctggccatcgctgggctccaggctgaggatgaggctgattattactgccagtcctatgacagaagcctggatggatgggttttcggcggagggaccacgctgaccgtcctgg

SEQ ID NO: 50 - protein, 111 aa, synthetic construct
  source 1..111: note = MPK30-v1 VL (aa)
QSVLTQPPSVSGAPGQRVTISCTGTSSNIGAGYDVHWYQQFPGRSPKLLIYRNTNRPSGVPDRFSGSKSGSSASLAIAGLQAEDEADYYCQSYDRSLDGWVFGGGTTLTVL

SEQ ID NO: 51 - protein, 11 aa, synthetic construct
  source 1..11: note = MPK30-v1; MPK153; MPK152 CDR-L3 (aa)
QSYDRSLDGWV

SEQ ID NO: 52 - DNA, 358 bp, synthetic construct
  source 1..358: mol_type = other DNA; note = MPK36-v3 VH (nt)
gaggtgcagctggtggagtccgggggaggcttggtcaagcctggagggtccctgagactctcctgtgcagcctcattcaccatcagtggctactacatgatctggatccgccaggctccagggaaggggctggagtggatttcatacataagtggcagtagtacttacacaaattacgctgactctgtgaagggccgattcaccatctccagggacaacggccagaacacactgtatctccacatgaccagcctgagagccgacgacacggccctctattattgtgcgacagaacagacgccgtcacctggccccttcgacctctggggccagggaaccctggtcaccgtctcctcag

SEQ ID NO: 53 - protein, 119 aa, synthetic construct
  source 1..119: note = MPK36-v3 VH (aa)
EVQLVESGGGLVKPGGSLRLSCAASFTISGYYMIWIRQAPGKGLEWISYISGSSTYTNYADSVKGRFTISRDNGQNTLYLHMTSLRADDTALYYCATEQTPSPGPFDLWGQGTLVTVSS

SEQ ID NO: 54 - protein, 7 aa, synthetic construct
  source 1..7: note = MPK36-v3 CDR-H1 (aa)
FTISGYY

SEQ ID NO: 55 - DNA, 334 bp, synthetic construct
  source 1..334: mol_type = other DNA; note = MPK36-v3 VL (nt)
cagactgtggtgacgcagccgccctcagtgtctggggccccaggacagagggtcaccatctcctgctctgggaccagctccaacatcggggcaggttatgatgtacactggtaccaacagtttccaggaacatcccccaaactcctcatctatcgtaatttcaatcggccctcaggagtccctgaccgattctctggctccaagtctggctcctcagcctccctggccatcgctgggctccaggctgaggatgaggctgattattactgccagtcctatgacaaaagcctggatggatgggttttcggcggagggaccaaggtgaccgtcctag

SEQ ID NO: 56 - protein, 111 aa, synthetic construct
  source 1..111: note = MPK36-v3 VL (aa)
QTVVTQPPSVSGAPGQRVTISCSGTSSNIGAGYDVHWYQQFPGTSPKLLIYRNFNRPSGVPDRFSGSKSGSSASLAIAGLQAEDEADYYCQSYDKSLDGWVFGGGTKVTVL

SEQ ID NO: 57 - protein, 11 aa, synthetic construct
  source 1..11: note = MPK36-v3 CDR-L3 (aa)
QSYDKSLDGWV

SEQ ID NO: 58 - DNA, 361 bp, synthetic construct
  source 1..361: mol_type = other DNA; note = MPK51 VH (nt)
caggtgcagctggtggagtctgggggaggcttggtcaagcctggagggtccctgagactctcctgtgcagcctctggattcaggatcagtggctactacatgatctgggtccgccaggctcccgggaagggactggagtggatttcagccatcagtggtatgagttcttacataacttatgcagactctgtgaagggccgattcaccgtctccagagacaacggccagaattcactatttttggaaatgaaaagcctcagaggcgaggacacggctgtctatttctgtgcgagagaagaggtgccgtcaccaggcccctttgacttatggggccagggaaccccggtcaccgtctccccag

SEQ ID NO: 59 - protein, 120 aa, synthetic construct
  source 1..120: note = MPK51 VH (aa)
QVQLVESGGGLVKPGGSLRLSCAASGFRISGYYMIWVRQAPGKGLEWISAISGMSSYITYADSVKGRFTVSRDNGQNSLFLEMKSLRGEDTAVYFCAREEVPSPGPFDLWGQGTPVTVSP

SEQ ID NO: 60 - protein, 8 aa, synthetic construct
  source 1..8: note = MPK51; MPK51-v1.1 CDR-H1 (aa)
GFRISGYY

SEQ ID NO: 61 - protein, 8 aa, synthetic construct
  source 1..8: note = MPK51; MPK51-v1.1 CDR-H2 (aa)
ISGMSSYI

SEQ ID NO: 62 - protein, 13 aa, synthetic construct
  source 1..13: note = MPK51; MPK51-v1.1 CDR-H3 (aa)
AREEVPSPGPFDL

SEQ ID NO: 63 - DNA, 334 bp, synthetic construct
  source 1..334: mol_type = other DNA; note = MPK51 VL (nt)
cagtctgtcgtgacgcagccgccctcagtgtctggggccccagggcagagggtcaccatcttctgcactggaagtacctccaacatcggggcaggttacgatgtccactggtaccagcaccttccgggaacagccccccgactcctcatctttaggaacaacaatcggccctcaggggtccctgaccgattctctggattcaagtctggcacctcagcctccctggccatcattgggctccaggttgaagatgaggctgattattactgccagtcctatgacagaaacatggatggttgggcgttcggcggagggaccaagctgaccgtcctgg

SEQ ID NO: 64 - protein, 111 aa, synthetic construct
  source 1..111: note = MPK51 VL (aa)
QSVVTQPPSVSGAPGQRVTIFCTGSTSNIGAGYDVHWYQHLPGTAPRLLIFRNNNRPSGVPDRFSGFKSGTSASLAIIGLQVEDEADYYCQSYDRNMDGWAFGGGTKLTVL

SEQ ID NO: 65 - protein, 9 aa, synthetic construct
  source 1..9: note = MPK51 CDR-L1 (aa)
TSNIGAGYD

SEQ ID NO: 66 - skipped

SEQ ID NO: 67 - protein, 11 aa, synthetic construct
  source 1..11: note = MPK51 CDR-L3 (aa)
QSYDRNMDGWA

SEQ ID NO: 68 - DNA, 361 bp, synthetic construct
  source 1..361: mol_type = other DNA; note = MPK67 VH (nt)
gaggtgcagctggtggagtctgggggaggcttggtcaagcctggagggtccctaagactctcctgtgcagcctctggattcaccatcagtggctactacatgatctggatccgccaggctccagggaaggggctggagtggatttcatacataagtggcagtagttcttatacaaactacggtgcctctgtgaagggccgattcaccatctccagggacaacggccaaaacacagtgcatctccaaatgaccaacctgagagccgacgacacggccctctattactgtgcgacagaacagacgccgtcacctggccccttcgacctctggggccagggaaccctggtcaccgtctcctcag

SEQ ID NO: 69 - protein, 120 aa, synthetic construct
  source 1..120: note = MPK67 VH (aa)
EVQLVESGGGLVKPGGSLRLSCAASGFTISGYYMIWIRQAPGKGLEWISYISGSSSYTNYGASVKGRFTISRDNGQNTVHLQMTNLRADDTALYYCATEQTPSPGPFDLWGQGTLVTVSS

SEQ ID NO: 70 - DNA, 334 bp, synthetic construct
  source 1..334: mol_type = other DNA; note = MPK67 VL (nt)
cagtctgtgctgacgcagccgccctcagtgtctggggccccaggacagagggtcaccatctcctgctctgggaccagctccaacatcggggcaggttatgatgtacactggtaccaacagtttccaggaacatcccccaaactcctcatctctcgtaatttcaatcggccctcaggagtccctgcccgattctctggctccaagtctggctcctcagcctccctggccatcgctggactccagcctgaggatgaggctgattattactgccagtcctatgacagaagcctgggtggctgggttttcggcggagggaccaaggtgaccgtcctag

SEQ ID NO: 71 - protein, 111 aa, synthetic construct
  source 1..111: note = MPK67 VL (aa)
QSVLTQPPSVSGAPGQRVTISCSGTSSNIGAGYDVHWYQQFPGTSPKLLISRNFNRPSGVPARFSGSKSGSSASLAIAGLQPEDEADYYCQSYDRSLGGWVFGGGTKVTVL

SEQ ID NO: 72 - DNA, 358 bp, synthetic construct
  source 1..358: mol_type = other DNA; note = MPK77 VH (nt)
gaggtgcaactggtggagtctgggggaggcttggtcaagcctggagggtccctgagactctcctgtgcagcctctaaatccttcagcggctactacatgatctggatccgccaggctccagggagggggctggaatggattgcagacattagtggaagtagtacttacacagactacgctgactctgttaagggccgattcaccatctccagagacaacacccggaacacactgtatctccaaatgaacagtctgagacccgaagacacggctgtgtattattgtgcgacagaacaggtgccgtcacctggcccctttgacctctggggcccgggaaccctggtcaccgtctcctcag

SEQ ID NO: 73 - protein, 119 aa, synthetic construct
  source 1..119: note = MPK77 VH (aa)
EVQLVESGGGLVKPGGSLRLSCAASKSFSGYYMIWIRQAPGRGLEWIADISGSSTYTDYADSVKGRFTISRDNTRNTLYLQMNSLRPEDTAVYYCATEQVPSPGPFDLWGPGTLVTVSS

SEQ ID NO: 74 - protein, 7 aa, synthetic construct
  source 1..7: note = MPK77; MPK77-v1.1 CDR-H1 (aa)
KSFSGYY

SEQ ID NO: 75 - DNA, 334 bp, synthetic construct
  source 1..334: mol_type = other DNA; note = MPK77 VL (nt)
cagtctgtgctgacgcagccgccctcagtgtctggggccccagggcagagggtcatcatctcctgcactgggagtagctccaacatcggggcaggttatgatgtacactggtaccaacaatttccaggaacatcccccaaactcctcatctatcgtaataccaatcggccctcaggggtccctgaccgattctctggcttcaagtctggcgcctcagccgccctggccatcactgggctccaggctgaggacgaggctgattattactgtcagtcctatgacagaagcctgagtggatgggctttcggcggagggaccaaggtgaccgtcctag

SEQ ID NO: 76 - protein, 111 aa, synthetic construct
  source 1..111: note = MPK77 VL (aa)
QSVLTQPPSVSGAPGQRVIISCTGSSSNIGAGYDVHWYQQFPGTSPKLLIYRNTNRPSGVPDRFSGFKSGASAALAITGLQAEDEADYYCQSYDRSLSGWAFGGGTKVTVL

SEQ ID NO: 77 - protein, 11 aa, synthetic construct
  source 1..11: note = MPK77 CDR-L3 (aa)
QSYDRSLSGWA

SEQ ID NO: 78 - DNA, 361 bp, synthetic construct
  source 1..361: mol_type = other DNA; note = MPK86 VH (nt)
gaggtgcagctggtggagtctgggggaggcttggtcaaacctggagggtccctgagactctcctgcgcagcctctggattctccatctctggatactacatgatctggatccgccaggctccagggagggggctggagtggatttcagacattagtgccagctctacttacacagactacgccgactctgtgaagggccgattcaccatctccagagacaacggccaaaacacagtgtatctacaaatgaacaacctgagagccgatgacacggctctatattattgtgcgactgaacagacgccgtcacctggcccctttgacctctggggccagggaaccctggtcaccgtctcctcag

SEQ ID NO: 79 - protein, 120 aa, synthetic construct
  source 1..120: note = MPK86 VH (aa)
EVQLVESGGGLVKPGGSLRLSCAASGFSISGYYMIWIRQAPGRGLEWISDISASSTYTDYADSVKGRFTISRDNGQNTVYLQMNNLRADDTALYYCATEQTPSPGPFDLWGQGTLVTVSS

SEQ ID NO: 80 - protein, 8 aa, synthetic construct
  source 1..8: note = MPK86; MPK130 CDR-H1 (aa)
GFSISGYY

SEQ ID NO: 81 - protein, 8 aa, synthetic construct
  source 1..8: note = MPK86; MPK126; MPK151 CDR-H2 (aa)
ISASSTYT

SEQ ID NO: 82 - DNA, 334 bp, synthetic construct
  source 1..334: mol_type = other DNA; note = MPK86 VL (nt)
cagtctgtcgtgacgcagccgccctcagtgtctggggccccaggacagagggtcatcatctcctgctctgggcgcagctccaacatcggggcaggttatgatgttcactggtaccagcactttccaggaacatcccccaaactcctcatctatcgtaataccaatcggccctcaggggtccctgaccgcttctctgcctccacgtctgccacctcagcctccctggccatcactgggctccaggctgacgatgaggctgattattactgccagtcttatgacagaaacctgggtggatgggttttcggcggagggaccaagctgaccgtcctag

SEQ ID NO: 83 - protein, 111 aa, synthetic construct
  source 1..111: note = MPK86 VL (aa)
QSVVTQPPSVSGAPGQRVIISCSGRSSNIGAGYDVHWYQHFPGTSPKLLIYRNTNRPSGVPDRFSASTSATSASLAITGLQADDEADYYCQSYDRNLGGWVFGGGTKLTVL

SEQ ID NO: 84 - protein, 11 aa, synthetic construct
  source 1..11: note = MPK86 CDR-L3 (aa)
QSYDRNLGGWV

SEQ ID NO: 85 - DNA, 361 bp, synthetic construct
  source 1..361: mol_type = other DNA; note = MPK92 VH (nt)
gaggtgcagctggtggagtctgggggaggcttggtcaagcctggagggtccctaagactctcctgtgaagcctctggaatctccctcagtggatactacatgatctggatccgccaggctccagggaagggactggagtggatttcatccatcagtagtagtagtaattatattgactacgctgactctgtgaggggccgattcaccatctccagacacaacggccagaacacactctatctccagatgaacagcctgagagtcgaggacacggctgtttatttttgtgcgaccgagcaaacgccgtcccctggcccctttgaattgtggggccagggaatcctggtcaccgtctcatcag

SEQ ID NO: 86 - protein, 120 aa, synthetic construct
  source 1..120: note = MPK92 VH (aa)
EVQLVESGGGLVKPGGSLRLSCEASGISLSGYYMIWIRQAPGKGLEWISSISSSSNYIDYADSVRGRFTISRHNGQNTLYLQMNSLRVEDTAVYFCATEQTPSPGPFELWGQGILVTVSS

SEQ ID NO: 87 - protein, 8 aa, synthetic construct
  source 1..8: note = MPK92; MPK132-v1; MPK150-v2; MPK155 CDR-H1 (aa)
GISLSGYY

SEQ ID NO: 88 - protein, 8 aa, synthetic construct
  source 1..8: note = MPK92; MPK132-v1; MPK155; MPK153; MPK204 CDR-H2 (aa)
ISSSSNYI

SEQ ID NO: 89 - protein, 13 aa, synthetic construct
  source 1..13: note = MPK92; MPK132-v1; MPK150-v2 CDR-H3 (aa)
ATEQTPSPGPFEL

SEQ ID NO: 90 - DNA, 334 bp, synthetic construct
  source 1..334: mol_type = other DNA; note = MPK92 VL (nt)
cagtctgtgttgacgcagccgccctcagtgtctggggccccagggcagagggtcaccatcttctgcactgggaccacctccaacatcgggacaggttatgatgtacactggtaccaacagtttccaggaacatcccccaaactcctcatctatcgtaataccaatcggccctcaggggtccctgaccgattctctggctccaggtctggctcctcagcctccctggccatcgctgggctccaggctgaggatgaggctgattattactgccagtcgtttgacagaagtgtcaatggatggattttcggcggagggaccaaggtgaccgtcctgg

SEQ ID NO: 91 - protein, 111 aa, synthetic construct
  source 1..111: note = MPK92 VL (aa)
QSVLTQPPSVSGAPGQRVTIFCTGTTSNIGTGYDVHWYQQFPGTSPKLLIYRNTNRPSGVPDRFSGSRSGSSASLAIAGLQAEDEADYYCQSFDRSVNGWIFGGGTKVTVL

SEQ ID NO: 92 - protein, 9 aa, synthetic construct
  source 1..9: note = MPK92 CDR-L1 (aa)
TSNIGTGYD

SEQ ID NO: 93 - protein, 11 aa, synthetic construct
  source 1..11: note = MPK92 CDR-L3 (aa)
QSFDRSVNGWI

SEQ ID NO: 94 - DNA, 361 bp, synthetic construct
  source 1..361: mol_type = other DNA; note = MPK99 VH (nt)
gaggtgcagctggtggagtctgggggaggcttggtcaagccgggagggtccctcagactctcctgtgcagcctctggattcaccatcagtggctactacatgatctggatccgccagtctccagggaagggactggagtggatttcagacattagtgctagtagttcttacacagactacgctgactctgtgaagggccgattcaccatctccagggacaacggccagaacacagtttatctccagatgaacagcctgagagccgaggacacggctgtctactactgtgcgacagaacaggtgccgtcacctggccccttcgacctctggggccagggaaccctggtcaccgtctcctcag

SEQ ID NO: 95 - protein, 120 aa, synthetic construct
  source 1..120: note = MPK99 VH (aa)
EVQLVESGGGLVKPGGSLRLSCAASGFTISGYYMIWIRQSPGKGLEWISDISASSSYTDYADSVKGRFTISRDNGQNTVYLQMNSLRAEDTAVYYCATEQVPSPGPFDLWGQGTLVTVSS

SEQ ID NO: 96 - protein, 8 aa, synthetic construct
  source 1..8: note = MPK99 CDR-H2 (aa)
ISASSSYT

SEQ ID NO: 97 - DNA, 334 bp, synthetic construct
  source 1..334: mol_type = other DNA; note = MPK99 VL (nt)
cagtctgtcgtgacgcagccgccctcggtgtctggggccccagggctgagggtcaccatctcctgctctgggaccagctccaacatcgggagaggttatgatgtgcactggtaccagcagtttccaggacagtcccccaaactcctcatctatcgtaacaccaatcggccctcaggggtccctgaccggttctccggcttcaagtctggctcttcagcctccctggccatcgctgggctccaggctgaggatgaggctgattattactgccagtcctatgacagaagcctgggtggatgggttttcggcggcgggaccaggctgatcgtcctag

SEQ ID NO: 98 - protein, 111 aa, synthetic construct
  source 1..111: note = MPK99 VL (aa)
QSVVTQPPSVSGAPGLRVTISCSGTSSNIGRGYDVHWYQQFPGQSPKLLIYRNTNRPSGVPDRFSGFKSGSSASLAIAGLQAEDEADYYCQSYDRSLGGWVFGGGTRLIVL

SEQ ID NO: 99 - protein, 9 aa, synthetic construct
  source 1..9: note = MPK99 CDR-L1 (aa)
SSNIGRGYD

SEQ ID NO: 100 - DNA, 352 bp, synthetic construct
  source 1..352: mol_type = other DNA; note = MPK15 VH (nt)
gaggtgcagctggtggagtctgggggaggcttggtaaagccgggggggtcccttagactctcctgcacagtctctggaatcactttcagtgacgcctggatgacctgggtccgccaggctccaggaaagggactggagtgggttggtcgtattaaaaggaaagttgatggagagacagcagactacgcgccatccgtgaaaggaagattcaccatctcaagagatgattcaaaaagcaccctatatctgcaaatgaacagcctgaaaaccgaagacacaggcctgtattactgtctcacgacatggagcgatcaattctggggccagggaaccctggtcaccgtctcctcgg

SEQ ID NO: 101 - protein, 117 aa, synthetic construct
  source 1..117: note = MPK15 VH (aa)
EVQLVESGGGLVKPGGSLRLSCTVSGITFSDAWMTWVRQAPGKGLEWVGRIKRKVDGETADYAPSVKGRFTISRDDSKSTLYLQMNSLKTEDTGLYYCLTTWSDQFWGQGTLVTVSS

SEQ ID NO: 102 - protein, 8 aa, synthetic construct
  source 1..8: note = MPK15 CDR-H1 (aa)
GITFSDAW

SEQ ID NO: 103 - protein, 10 aa, synthetic construct
  source 1..10: note = MPK15 CDR-H2 (aa)
IKRKVDGETA

SEQ ID NO: 104 - protein, 8 aa, synthetic construct
  source 1..8: note = MPK15 CDR-H3 (aa)
LTTWSDQF

SEQ ID NO: 105 - DNA, 328 bp, synthetic construct
  source 1..328: mol_type = other DNA; note = MPK15 VL (nt)
cagtctgtcgtgacgcaggagccctcactgactgtgtccccaggagggacagtcactctcacctgtgactccagtagtggagctgtcaccagaggtcattatcccttctggttccagcagaagccaggccaagttcccaggacactcatttatgatacaagcaagaaacactcctggacccctgcccggttctcaggctccctccttggggacaaagctgtcctgaccctttcgggtgcgcagcctgaggatgaggctgattattattgcttactcacctacagtggtgttcgggtgttcggcggagggaccaagctgaccgtcctag

SEQ ID NO: 106 - protein, 109 aa, synthetic construct
  source 1..109: note = MPK15 VL (aa)
QSVVTQEPSLTVSPGGTVTLTCDSSSGAVTRGHYPFWFQQKPGQVPRTLIYDTSKKHSWTPARFSGSLLGDKAVLTLSGAQPEDEADYYCLLTYSGVRVFGGGTKLTVL

SEQ ID NO: 107 - protein, 9 aa, synthetic construct
  source 1..9: note = MPK15 CDR-L1 (aa)
SGAVTRGHY

SEQ ID NO: 108 - skipped

SEQ ID NO: 109 - protein, 9 aa, synthetic construct
  source 1..9: note = MPK15 CDR-L3 (aa)
LLTYSGVRV

SEQ ID NO: 110 - DNA, 358 bp, synthetic construct
  source 1..358: mol_type = other DNA; note = MPK104 VH (nt)
gaggtgcagctggtggagtctgggggaggcctggtcaagcctggggggtccctgagactctcctgtgcagcctctggattcaccatcgatggctatggcatgaactgggtccgccaggctccagggaaggggctggagtgggtctcttccatcagcgctagtagcaattacatatattatgcagactcaatgaagggccggttcaccatctccagagacaacggcaagaattcactctatctgcaaatgcacagcctgagagtcgaggacacgggtgtctattattgtgcgagagcgcctctagaaccgccatacttcgatctctggggccgtggcaccctggtcactgtctcctcag

SEQ ID NO: 111 - protein, 119 aa, synthetic construct
  source 1..119: note = MPK104 VH (aa)
EVQLVESGGGLVKPGGSLRLSCAASGFTIDGYGMNWVRQAPGKGLEWVSSISASSNYIYYADSMKGRFTISRDNGKNSLYLQMHSLRVEDTGVYYCARAPLEPPYFDLWGRGTLVTVSS

SEQ ID NO: 112 - protein, 8 aa, synthetic construct
  source 1..8: note = MPK104; MPK104-v1.1; MPK104-v1.3 CDR-H1 (aa)
GFTIDGYG

SEQ ID NO: 113 - protein, 8 aa, synthetic construct
  source 1..8: note = MPK104; MPK104-v1.1; MPK104-v1.3 CDR-H2 (aa)
ISASSNYI

SEQ ID NO: 114 - protein, 12 aa, synthetic construct
  source 1..12: note = MPK104; MPK104-v1.1; MPK104-v1.3 CDR-H3 (aa)
ARAPLEPPYFDL

SEQ ID NO: 115 - DNA, 334 bp, synthetic construct
  source 1..334: mol_type = other DNA; note = MPK104 VL (nt)
cagtctgtgctgacgcagccgccctcagtgtctggggccccaggccagagggtcaccatctcctgcagcgggggtagttccaacatcggggcaggttatgatgtgcattggtaccagcaggtgccaggaaaaggccccaaagtcctcatctatggcaacaacaatcggccctcaggggtccctgaccgattctctggctctaattttggcgcctcagcctccctggccatcactggactccaggctgacgatgaggctgattatttctgccagtcttatgaccggagactgggtggtccggtattcggcggagggaccaagctgaccgtcctag

SEQ ID NO: 116 - protein, 111 aa, synthetic construct
  source 1..111: note = MPK104; MPK104-v1.1 VL (aa)
QSVLTQPPSVSGAPGQRVTISCSGGSSNIGAGYDVHWYQQVPGKGPKVLIYGNNNRPSGVPDRFSGSNFGASASLAITGLQADDEADYFCQSYDRRLGGPVFGGGTKLTVL

SEQ ID NO: 117 - skipped

SEQ ID NO: 118 - protein, 11 aa, synthetic construct
  source 1..11: note = MPK104; MPK104-v1.1; MPK104-v1.3 CDR-L3 (aa)
QSYDRRLGGPV

SEQ ID NO: 119 - DNA, 358 bp, synthetic construct
  source 1..358: mol_type = other DNA; note = MPK73 VH (nt)
gaggtgcagctggtggagtcggggggaggcctggtcaagccgggggggtccctgacactctcctgtgcagcctctggattctccatcagtggatatggcatgaactgggtccgccaggctccagggaggggactggagtgggtcgcatccattagtgccagtagtacttacatatattatgcagactcaatgaagggccgattcaccatctccagagacaacggcaagacttcactctatctgcaaatgaagagtttgcgagccgaggacacggctgtctattattgtgcgagagcgcctatagaaccgccgtacttcgatctctggggccgtggcaccctggtcactgtctcctcag

SEQ ID NO: 120 - protein, 119 aa, synthetic construct
  source 1..119: note = MPK73 VH (aa)
EVQLVESGGGLVKPGGSLTLSCAASGFSISGYGMNWVRQAPGRGLEWVASISASSTYIYYADSMKGRFTISRDNGKTSLYLQMKSLRAEDTAVYYCARAPIEPPYFDLWGRGTLVTVSS

SEQ ID NO: 121 - protein, 8 aa, synthetic construct
  source 1..8: note = MPK73; MPK190; MPK190-v1.1; MPK190- v1.3 CDR-H1 (aa)
GFSISGYG

SEQ ID NO: 122 - protein, 8 aa, synthetic construct
  source 1..8: note = MPK73; MPK190; MPK190-v1.1; MPK190- v1.3 CDR-H2 (aa)
ISASSTYI

SEQ ID NO: 123 - protein, 12 aa, synthetic construct
  source 1..12: note = MPK73; MPK 190; MPK190-v1.1; MPK190- v1.3 CDR-H3 (aa)
ARAPIEPPYFDL

SEQ ID NO: 124 - DNA, 334 bp, synthetic construct
  source 1..334: mol_type = other DNA; note = MPK73 VL (nt)
cagtctgtgctgacgcagccgccctcagtgtctggggcccccgggcagagggtcaccatctcctgcacagggagtaactccaacatcggggcaggatatgatgttcactggtaccaacatcttccaggaaaagcccccaaagtcctcatctatggtaacaacaatcggccctcaggggtccctgaccgattctctggctccaactctggcgcctcagcctccctggccatcagtgggctccaggctgaggatgaggctgattatttctgccagtcttatgacaggagactgagtggtccgatcttcggcggagggaccaagctgaccgtcctag

SEQ ID NO: 125 - protein, 111 aa, synthetic construct
  source 1..111: note = MPK73 VL (aa)
QSVLTQPPSVSGAPGQRVTISCTGSNSNIGAGYDVHWYQHLPGKAPKVLIYGNNNRPSGVPDRFSGSNSGASASLAISGLQAEDEADYFCQSYDRRLSGPIFGGGTKLTVL

SEQ ID NO: 126 - protein, 9 aa, synthetic construct
  source 1..9: note = MPK73; MPK190; MPK190-v1.1 CDR-L1 (aa)
NSNIGAGYD

SEQ ID NO: 127 - protein, 11 aa, synthetic construct
  source 1..11: note = MPK73; MPK190; MPK190-v1.1; MPK190- v1.3 CDR-L3 (aa)
QSYDRRLSGPI

SEQ ID NO: 128 - DNA, 358 bp, synthetic construct
  source 1..358: mol_type = other DNA; note = MPK9 VH (nt)
gaggtgcagctggtggagtctgggggaggcctggtcaagccgggggggtccctgagactctcctgtgcagcctctggattcaccatcagtggctatggcatgtcctggttccgccaggctccagggaaggggctggagtgggtctcatccattagtgctagtagtagttacatatattatgcagactcaacgaagggccgattcaccatctccagagacaacggcaagcaatcactgtatctgcaaatgaacagcctgagagccgaggacacggctgtctattattgtgcgacatcgcctatagagccgccgtacttcgatctctggggccgtggcaccctggtcactgtctcctcag

SEQ ID NO: 129 - protein, 119 aa, synthetic construct
  source 1..119: note = MPK9 VH (aa)
EVQLVESGGGLVKPGGSLRLSCAASGFTISGYGMSWFRQAPGKGLEWVSSISASSSYIYYADSTKGRFTISRDNGKQSLYLQMNSLRAEDTAVYYCATSPIEPPYFDLWGRGTLVTVSS

SEQ ID NO: 130 - protein, 8 aa, synthetic construct
  source 1..8: note = MPK9; MPK142-v1; MPK142-v2; MPK145 CDR-H1 (aa)
GFTISGYG

SEQ ID NO: 131 - protein, 12 aa, synthetic construct
  source 1..12: note = MPK9 CDR-H3 (aa)
ATSPIEPPYFDL

SEQ ID NO: 132 - DNA, 334 bp, synthetic construct
  source 1..334: mol_type = other DNA; note = MPK9 VL (nt)
cagtctgtgctgacgcagccgccctcagtgtctggggccccagggcagagggtcaccatctcctgcactgggagtagctccaacatcggggcaggttatgatgtgcactggtaccagcagcttccaggaaaagcccccaaactcctcatctatggtaacaacaatcggccctcaggggtccctgaccgattctctggctccaactctggcgcctcagcctccctggccatcactgggctccaggctgaggatgaggctgattatttctgccagtcctatgacagaagactgagtggtccggtattcggcagagggaccaagctgaccgtcctag

SEQ ID NO: 133 - protein, 111 aa, synthetic construct
  source 1..111: note = MPK9 VL (aa)
QSVLTQPPSVSGAPGQRVTISCTGSSSNIGAGYDVHWYQQLPGKAPKLLIYGNNNRPSGVPDRFSGSNSGASASLAITGLQAEDEADYFCQSYDRRLSGPVFGRGTKLTVL

SEQ ID NO: 134 - protein, 11 aa, synthetic construct
  source 1..11: note = MPK9; MPK142-v1; MPK145 CDR-L3 (aa)
QSYDRRLSGPV

SEQ ID NO: 135 - DNA, 391 bp, synthetic construct
  source 1..391: mol_type = other DNA; note = MPK65-v2 VH (nt) (VH.1)(non-optimized)
caggtcccgctggtgcagtctggagcagaggtgaaaaagcccggggagtctctgaagatctcctgtaagggttctggatacagctttattaattactggatcggctgggtgcgccagatgcccgggaaaggcctggagtggatgggagtcatctatccttttgactctgataccaaatacagcccgtccttccaaggccacgtcaccatctcagccgacaagtccagcaacaccgcctacgtgcagtggagcagcctgaaggcctcggacaccgccatgtattactgtgcgagatttacctttggtattgcagtggtcacagatgatgttagtggggaaggccaagcttttgaaatctggggccaagggacaatggtcaccgtctcttcag

SEQ ID NO: 136 - protein, 130 aa, synthetic construct
  source 1..130: note = MPK65-v2 VH (aa) (VH.1)
QVPLVQSGAEVKKPGESLKISCKGSGYSFINYWIGWVRQMPGKGLEWMGVIYPFDSDTKYSPSFQGHVTISADKSSNTAYVQWSSLKASDTAMYYCARFTFGIAVVTDDVSGEGQAFEIWGQGTMVTVSS

SEQ ID NO: 137 - protein, 8 aa, Synthetic construct
  source 1..8: note = MPK65-v2 VH.1; MPK167; MPK169-v2; MPK170; MPK171-v1; MPK171-v2; MPK175; MPK179-v4; MPK181; MPK186; MPK188; MPK189; MPK191; MPK195; MPK198; MPK201; MPK201 VH.2; MPK201 VH.3 CDR-H1 (aa)
GYSFINYW

SEQ ID NO: 138 - protein, 8 aa, synthetic construct
  source 1..8: note = MPK65-v2 VH.1; MPK65 VH.4; MPK65 VH.5; MPK161-v1; MPK161-v2; MPK165; MPK167; MPK168; MPK169-v2; MPK170; MPK171-v1; MPK171-v2; MPK177; MPK178; MPK179-v4; MPK181; MPK185; MPK188; MPK189; MPK191; MPK193; MPK195; MPK198 CDR-H2 (aa)
IYPFDSDT

SEQ ID NO: 139 - protein, 23 aa, synthetic construct
  source 1..23: note = MPK65-v2; MPK65 VH.2; MPK65 VH.3; VH.1 CDR-H3 (aa)
ARFTFGIAVVTDDVSGEGQAFEI

SEQ ID NO: 140 - DNA, 322 bp, synthetic construct
  source 1..322: mol_type = other DNA; note = MPK65-v2 VK.1 VL (nt)
gaaatagtgatgatgcagtctccagccaccctgtctgtgtctccaggggaaagagccaccctctcctgcagggccagtcagagtgttgccggcaacttagcctggtaccagcagagacctggccaggctcccaggctcctcatttatggtgcatccaccagggccactggtgtcccagccaggttcagtggcagtgggtctgagacagagttcactctcaccatcagcagcctgcagtctgaagattttgcagtttattactgtcaccagtatagtaactggcctcaagccttcggccaagggacacgactggacattaaac

SEQ ID NO: 141 - protein, 107 aa, synthetic construct
  source 1..107: note = MPK65-v2 VK.1 VL (aa)
EIVMMQSPATLSVSPGERATLSCRASQSVAGNLAWYQQRPGQAPRLLIYGASTRATGVPARFSGSGSETEFTLTISSLQSEDFAVYYCHQYSNWPQAFGQGTRLDIK

SEQ ID NO: 142 - protein, 6 aa, synthetic construct
  source 1..6: note = MPK65-v2 VK.1; MPK65 VK.2; MPK161-v2; MPK167; MPK170; MPK171-v2; MPK178; MPK189; MPK191; MPK193; MPK198; MPK201; MPK201 VK.2 CDR-L1 (aa)
QSVAGN

SEQ ID NO: 143 - skipped

SEQ ID NO: 144 - protein, 9 aa, synthetic construct
  source 1..9: note = MPK65-v2 VK.1 CDR-L3 (aa)
HQYSNWPQA

SEQ ID NO: 145 - DNA, 391 bp, synthetic construct
  source 1..391: mol_type = other DNA; note = MPK161-v1 VH (nt) and MPK161-v2 VH (nt)
gaggtgcaactggtgcagtctggagtagaggcgaaaaagcccggggagtctctgaagatctcctgtcgggcttctggatacagttttgttaatttctggatcggctgggtgcgccaggtgcccgggaaaggcctggagtggatgggattcatctatccttttgattctgataccaaatatggtccgtccttcgaaggtcacgtcatcatctcagccgacaagtccaccaacaccgcctacttggagtggagaagcctgagggcctcggacagcgccatgtattactgtgcgagatttacctttggtattgcattagtcgcaggtgatgttactgggggaggccaggcttttgaagtctggggccaagggactttggtcagcgtgtcttcag

SEQ ID NO: 146 - protein, 130 aa, synthetic construct
  source 1..130: note = MPK161-v1 VH (aa)
EVQLVQSGVEAKKPGESLKISCRASGYSFVNFWIGWVRQVPGKGLEWMGFIYPFDSDTKYGPSFEGHVIISADKSTNTAYLEWRSLRASDSAMYYCARFTFGIALVAGDVTGGGQAFEVWGQGTLVSVSS

SEQ ID NO: 147 - protein, 8 aa, synthetic construct
  source 1..8: note = MPK161-v1 CDR-H1 (aa) and MPK161-v2-CRD-H1 (aa)
GYSFVNFW

SEQ ID NO: 148 - protein, 23 aa, synthetic construct
  source 1..23: note = MPK161-v1; MPK161-v2; MPK165; MPK178 CDR-H3 (aa)
ARFTFGIALVAGDVTGGGQAFEV

SEQ ID NO: 149 - DNA, 322 bp, synthetic construct
  source 1..322: mol_type = other DNA; note = MPK161-v1 VL (nt)
gccatctggatgacccagtctccttccaccctgtctgcttctgtaggagacagagtcactgtcacttgccgggccagtctgagtattggtacctggttggcctggtatcagcataagccagggaaagcccctaaattcttgatttataaggcgtccagtttggaaagtggggtcccatcaaggttcagcggcagcggatccgggacagaattcactctcaccatcagcagcctgcagcctgatgactttgcaacttattactgccaacagtatgacagttatccgttcaccttcggcggagggaccaaggtggagatcaaac

SEQ ID NO: 150 - protein, 107 aa, synthetic construct
  source 1..107: note = MPK161-v1 VL (aa)
AIWMTQSPSTLSASVGDRVTVTCRASLSIGTWLAWYQHKPGKAPKFLIYKASSLESGVPSRFSGSGSGTEFTLTISSLQPDDFATYYCQQYDSYPFTFGGGTKVEIK

SEQ ID NO: 151 - protein, 6 aa, synthetic construct
  source 1..6: note = MPK161-v1 CDR-L1 (aa)
LSIGTW

SEQ ID NO: 152 - skipped

SEQ ID NO: 153 - protein, 9 aa, synthetic construct
  source 1..9: note = MPK161-v1 CDR-L3 (aa)
QQYDSYPFT

SEQ ID NO: 154 - DNA, 322 bp, synthetic construct
  source 1..322: mol_type = other DNA; note = MPK161-v2 VL (nt)
gaaatagtgatgatgcagtctccagccaccctgtctgtgtctccaggggacagagccaccctctcttgcagggccagtcagagtgttgccggcaacttagcctggtaccagcagagacctggccaggctcccaggctcctcatttatgctgcatccaccagggccactggtgtcccagccaggttcagtggcagtgggtctgagacagagttcactctcaccatcagcagcctgcagtctgaagattttgcagtttattactgtcaccattatagtaactggcctcaagccttcggccaagggacacgactagacattaaac

SEQ ID NO: 155 - protein, 107 aa, synthetic construct
  source 1..107: note = MPK161-v2 VL (aa)
EIVMMQSPATLSVSPGDRATLSCRASQSVAGNLAWYQQRPGQAPRLLIYAASTRATGVPARFSGSGSETEFTLTISSLQSEDFAVYYCHHYSNWPQAFGQGTRLDIK

SEQ ID NO: 156 - skipped

SEQ ID NO: 157 - protein, 9 aa, synthetic construct
  source 1..9: note = MPK161-v2; MPK165; MPK170; MPK178; MPK188; MPK193 CDR-L3 (aa)
HHYSNWPQA

SEQ ID NO: 158 - DNA, 396 bp, synthetic construct
  source 1..396: mol_type = other DNA; note = MPK163 VH (nt)
gaggtgcagctggtgcagtctggagcagaggtgaagaagcccggggagtctctgaagatgtcctgtaagacttctggatacagctttcgcagttattggatcgcctgggtgcgccagaggcccgggaaaggcctggagtggatggggatcatctggcctggtgactctgatgtcagatacggcccgtccttccagggccaagtcaccatttcagccgacaggtccaccaataccgcctacctgcagtggagcagcctgaaggcctcggacaccgccatttattggtgtgcgagacagaattacgacttttggagtggtcattcgcttgggactggcgccgactactactatggtatggacgtctggggccaagggaccacggtcaccgtctcgtca

SEQ ID NO: 159 - protein, 132 aa, synthetic construct
  source 1..132: note = MPK163 VH (aa)
EVQLVQSGAEVKKPGESLKMSCKTSGYSFRSYWIAWVRQRPGKGLEWMGIIWPGDSDVRYGPSFQGQVTISADRSTNTAYLQWSSLKASDTAIYWCARQNYDFWSGHSLGTGADYYYGMDVWGQGTTVTVSS

SEQ ID NO: 160 - protein, 8 aa, synthetic construct
  source 1..8: note = MPK163 CDR-H1 (aa)
GYSFRSYW

SEQ ID NO: 161 - protein, 8 aa, synthetic construct
  source 1..8: note = MPK163 CDR-H2 (aa)
IWPGDSDV

SEQ ID NO: 162 - protein, 25 aa, synthetic construct
  source 1..25: note = MPK163 CDR-H3 (aa)
ARQNYDFWSGHSLGTGADYYYGMDV

SEQ ID NO: 163 - DNA, 331 bp, synthetic construct
  source 1..331: mol_type = other DNA; note = MPK163 VL (nt)
tcttctgagctgactcagccaccctcagcgtctgggacccccgggcagagagtcaccatctcttgttctggaagcagctccaacatcggaaggaatattgtacattggtaccagcacgtcccaggaacggcccccaaactcctcatctataataatgatgagcggccctcaggggtccctgaccgattctctggctccaagtctggcacctcagcctccctggccatcagtggactccagtctgaggatgaggctgattattattgtacagtatgggatgacagcctgaatggaggggtgttcggcggagggaccaaggtgaccgtcctac

SEQ ID NO: 164 - protein, 110 aa, synthetic construct
  source 1..110: note = MPK163 VL (aa)
SSELTQPPSASGTPGQRVTISCSGSSSNIGRNIVHWYQHVPGTAPKLLIYNNDERPSGVPDRFSGSKSGTSASLAISGLQSEDEADYYCTVWDDSLNGGVFGGGTKVTVL

SEQ ID NO: 165 - protein, 8 aa, synthetic construct
  source 1..8: note = MPK163 CDR-L1 (aa)
SSNIGRNI

SEQ ID NO: 166 - skipped

SEQ ID NO: 167 - protein, 11 aa, synthetic construct
  source 1..11: note = MPK163 CDR-L3 (aa)
TVWDDSLNGGV

SEQ ID NO: 168 - DNA, 391 bp, synthetic construct
  source 1..391: mol_type = other DNA; note = MPK165 VH (nt)
gaggtgcagctggtgcagtctggagcagaggtgaaaaagcccggggagtctctgaagatctcctgtagggcttctggatacagttttaataattactgggtcggctgggtgcgccaggtgcccgggaaaggcctggagtggatgggattcatctatccctttgactctgataccaaatacagtccgtccttcgaaggccacgtcaccatctcagccgacaagtccatcgacaccgcctacctgcagtggagcagcctgaggtcctcggacaccgccatatattactgtgcgagatttacctttggtattgcattggtcgcaggtgatgttactgggggaggccaggcttttgaagtctggggccaagggacgttggtcaccgtctcttcag

SEQ ID NO: 169 - protein, 130 aa, synthetic construct
  source 1..130: note = MPK165 VH (aa)
EVQLVQSGAEVKKPGESLKISCRASGYSFNNYWVGWVRQVPGKGLEWMGFIYPFDSDTKYSPSFEGHVTISADKSIDTAYLQWSSLRSSDTAIYYCARFTFGIALVAGDVTGGGQAFEVWGQGTLVTVSS

SEQ ID NO: 170 - protein, 8 aa, synthetic construct
  source 1..8: note = MPK165; MPK193 CDR-H1 (aa)
GYSFNNYW

SEQ ID NO: 171 - DNA, 322 bp, synthetic construct
  source 1..322: mol_type = other DNA; note = MPK165 VL (nt)
gaaattgtgatgacgcagtctccagccaccctgtctgtgtctccaggggagagagccaccctctcttgcagggccagtcagagtgttgccggcaagttagcctggtaccagcagagacctggccaggctcccaggctcatcatttatgctgcatccaccagggccgctggtgtcccagccaggttcagtggcagtgggtctgagacagagttcactctcaccatcagcagcctgcagtctgaagattttgcagtttattactgtcaccattatagtaactggcctcaagccttcggccaagggacacgactagacattaaac

SEQ ID NO: 172 - protein, 107 aa, synthetic construct
  source 1..107: note = MPK165 VL (aa)
EIVMTQSPATLSVSPGERATLSCRASQSVAGKLAWYQQRPGQAPRLIIYAASTRAAGVPARFSGSGSETEFTLTISSLQSEDFAVYYCHHYSNWPQAFGQGTRLDIK

SEQ ID NO: 173 - protein, 6 aa, synthetic construct
  source 1..6: note = MPK165; MPK177; MPK181; MPK195 CDR-L1 (aa)
QSVAGK

SEQ ID NO: 174 - DNA, 391 bp, synthetic construct
  source 1..391: mol_type = other DNA; note = MPK167 VH (nt)
gaggtgcagctggtgcagtctggaccagaggtgaaaaagcccggggagtctctgaagatctcctgtaaggcttctggatacagctttattaattattggatcggctgggtgcgccagatgcccgggaaaggcctggagtggatgggtatcatctatccttttgactctgatacccagtacagcccgtccttccaaggccacgtcaccatctctgccgacaagtccatcaacaccgcctacgtgcagtggagcagccttaaggcctcggacaccgccatgtattactgtgcgagatttacctttggtattgcagtggtcgcaggtgatgttagtggggaaggccatgcttttgatatctggggccaagggacgatggtcaccgtctcttcag

SEQ ID NO: 175 - protein, 130 aa, synthetic construct
  source 1..130: note = MPK167 VH (aa)
EVQLVQSGPEVKKPGESLKISCKASGYSFINYWIGWVRQMPGKGLEWMGIIYPFDSDTQYSPSFQGHVTISADKSINTAYVQWSSLKASDTAMYYCARFTFGIAVVAGDVSGEGHAFDIWGQGTMVTVSS

SEQ ID NO: 176 - protein, 23 aa, synthetic construct
  source 1..23: note = MPK167 CDR-H3 (aa)
ARFTFGIAVVAGDVSGEGHAFDI

SEQ ID NO: 177 - DNA, 322 bp, synthetic construct
  source 1..322: mol_type = other DNA; note = MPK167 VL (nt)
gaaatagtgatgatgcagtctccagccaccctgtctgtgtctccaggggaaagagccaccctctcctgcagggccagtcagagtgttgccggcaacttagcctggtaccagcagagccctggccaggctcccaggctcctcatttatggtgcatccaccagggccactggtgtcccagccaggttcagtggcagtgggtctgagacagagttcactctcaccatcagcagcctgcagtctgaagattttgcagtttatttctgtcagcagtatagtaactggcctcacgccttcggccaagggacacgactggacattaaac

SEQ ID NO: 178 - protein, 107 aa, synthetic construct
  source 1..107: note = MPK167 VL (aa)
EIVMMQSPATLSVSPGERATLSCRASQSVAGNLAWYQQSPGQAPRLLIYGASTRATGVPARFSGSGSETEFTLTISSLQSEDFAVYFCQQYSNWPHAFGQGTRLDIK

SEQ ID NO: 179 - protein, 9 aa, synthetic construct
  source 1..9: note = MPK167 CDR-L3 (aa)
QQYSNWPHA

SEQ ID NO: 180 - DNA, 391 bp, synthetic construct
  source 1..391: mol_type = other DNA; note = MPK168 VH (nt)
gaggtgcagctggtgcagtctggagcagaggtgaaaaagcccggggagtctctgaagatctcctgtcaggtttctggatacagctttgttaattattggatcggctgggtgcgccaggtgcccgggaaaggcctggagtggatggggctcatctatccttttgactctgacacccaatatagcccgtccttccaaggacacgtcaccatctccgccgacacgtccatcacgaccgcctacctccagtggagcagcctgcaggcctcggacaccgccacatattactgtgcgagatttaccttcggtcttgcactagtcacagccggtcttaccgatgacgggcaagcttttgatttctggggccaagggacagtggtcaccgtctcttcag

SEQ ID NO: 181 - protein, 130 aa, synthetic construct
  source 1..130: note = MPK168 VH (aa)
EVQLVQSGAEVKKPGESLKISCQVSGYSFVNYWIGWVRQVPGKGLEWMGLIYPFDSDTQYSPSFQGHVTISADTSITTAYLQWSSLQASDTATYYCARFTFGLALVTAGLTDDGQAFDFWGQGTVVTVSS

SEQ ID NO: 182 - protein, 8 aa, synthetic construct
  source 1..8: note = MPK168; MPK178 CDR-H1 (aa)
GYSFVNYW

SEQ ID NO: 183 - protein, 23 aa, synthetic construct
  source 1..23: note = MPK168 CDR-H3 (aa)
ARFTFGLALVTAGLTDDGQAFDF

SEQ ID NO: 184 - DNA, 322 bp, synthetic construct
  source 1..322: mol_type = other DNA; note = MPK168 VL (nt)
gaaatagtgatgatgcagtctccagccaccctgtctgtgtctccaggggaaagagccaccctctcctgcagggccactgagagtgttgccggcaagttagcctggtaccagcagagacctggccagactcccaggctcctcatttatggtgcatccgccagggccactggtgtcccagccaggttcagtggcagtgggtctgagacagagttcactctcaccatcagcagcctgcagtctgaggattttgcagtttattactgtcagcagtatagtacctggcctcacgcctttggccaggggacacgactggacattaaac

SEQ ID NO: 185 - protein, 107 aa, synthetic construct
  source 1..107: note = MPK168 VL (aa)
EIVMMQSPATLSVSPGERATLSCRATESVAGKLAWYQQRPGQTPRLLIYGASARATGVPARFSGSGSETEFTLTISSLQSEDFAVYYCQQYSTWPHAFGQGTRLDIK

SEQ ID NO: 186 - protein, 6 aa, synthetic construct
  source 1..6: note = MPK168 CDR-L1 (aa)
ESVAGK

SEQ ID NO: 187 - protein, 9 aa, synthetic construct
  source 1..9: note = MPK168 CDR-L3 (aa)
QQYSTWPHA

SEQ ID NO: 188 - DNA, 391 bp, synthetic construct
  source 1..391: mol_type = other DNA; note = MPK169-v2 VH (nt)
gaggtgcagctggtgcaatctggagcagaggtgaaaaagcccggggaatctctgacgatctcctgtcagacttctggatacagttttattaattattggatcggctgggtgcgccagatgcccgggaaaggcctggagtggatgggagtcatctatccttttgactctgacaccgaatacagcccgtccttccgaggccacgtcaccatctcagccgacaagtccatcaataccgcctacctccagtggaacagtctgggggcctcggacaccgccatgtattactgtgccagatttacctttggtattgcactggtctcaggtgatgttaacggggaaggccatgcttttgatgtctggggccgagggacactggtcaccgtctcttcag

SEQ ID NO: 189 - protein, 130 aa, synthetic construct
  source 1..130: note = MPK169-v2 VH (aa)
EVQLVQSGAEVKKPGESLTISCQTSGYSFINYWIGWVRQMPGKGLEWMGVIYPFDSDTEYSPSFRGHVTISADKSINTAYLQWNSLGASDTAMYYCARFTFGIALVSGDVNGEGHAFDVWGRGTLVTVSS

SEQ ID NO: 190 - protein, 23 aa, synthetic construct
  source 1..23: note = MPK169-v2 CDR-H3 (aa)
ARFTFGIALVSGDVNGEGHAFDV

SEQ ID NO: 191 - DNA, 322 bp, synthetic construct
  source 1..322: mol_type = other DNA; note = MPK169-v2 VL (nt)
gaaattgtgctgactcagtctccacccaccctgtctgtgtctccagggcaaagagccaccctctcctgcagggccagtcagagtgttgccgacaagttagcctggtaccagcagagacctggccaggctcccagactcctcatttatggtgcatcctccagggccactggtgtcccagccaggttcagtggcagtgggtctgagacagagttcactctcaccatcaccagcctgcaatctgaagattttgcactttattactgtcaccagtacaataactggcctcacgccttcggccaagggacacgactggtcattaaac

SEQ ID NO: 192 - protein, 107 aa, synthetic construct
  source 1..107: note = MPK169-v2 VL (aa)
EIVLTQSPPTLSVSPGQRATLSCRASQSVADKLAWYQQRPGQAPRLLIYGASSRATGVPARFSGSGSETEFTLTITSLQSEDFALYYCHQYNNWPHAFGQGTRLVIK

SEQ ID NO: 193 - protein, 6 aa, synthetic construct
  source 1..6: note = MPK169-v2 CDR-L1 (aa)
QSVADK

SEQ ID NO: 194 - protein, 9 aa, synthetic construct
  source 1..9: note = MPK169-v2 CDR-L3 (aa)
HQYNNWPHA

SEQ ID NO: 195 - DNA, 391 bp, synthetic construct
  source 1..391: mol_type = other DNA; note = MPK170 VH (nt)
caggtgcagctggtgcagtctggagcagaggtgaaaaagcccggggagtctctgaagatctcctgtaagggttctggatacagctttattaattactggatcggctgggtgcgccagatgcccgggaaaggcctggagtggatgggaatcatctatccttttgactctgataccaaatacagcccgtccttccaaggccacgtcaccatctcagcggacaagtccatcaacaccgcctacgtacagtggagcagcctgaaggcctcggacaccgccatttattactgtgcgagatttacgtttggtattgcagtgtccacagatgatgttagtggggaaggccaagcttttgatctctggggccaagggacaagggtcaccgtctcttcag

SEQ ID NO: 196 - protein, 130 aa, synthetic construct
  source 1..130: note = MPK170 VH (aa)
QVQLVQSGAEVKKPGESLKISCKGSGYSFINYWIGWVRQMPGKGLEWMGIIYPFDSDTKYSPSFQGHVTISADKSINTAYVQWSSLKASDTAIYYCARFTFGIAVSTDDVSGEGQAFDLWGQGTRVTVSS

SEQ ID NO: 197 - protein, 23 aa, synthetic construct
  source 1..23: note = MPK170 CDR-H3 (aa)
ARFTFGIAVSTDDVSGEGQAFDL

SEQ ID NO: 198 - DNA, 322 bp, synthetic construct
  source 1..322: mol_type = other DNA; note = MPK170 VL (nt)
gaaatagtgatgatgcagtctccagccaccctatctgtgtctccaggggaaagagccaccctctcctgcagggccagtcagagtgttgccggcaacttagcctggtaccagcagagacctggccaggctcccaggctcctcatttatgctggatccgccagggccactggtgtcccggccaggttcagtggcagtgggtctgagacagagttcactctcaccatcagcagcctgcagtctgaagattttgcagtttattactgtcaccactatagtaactggcctcaagccttcggccaagggacacgactggacattaaac

SEQ ID NO: 199 - protein, 107 aa, synthetic construct
  source 1..107: note = MPK170 VL (aa)
EIVMMQSPATLSVSPGERATLSCRASQSVAGNLAWYQQRPGQAPRLLIYAGSARATGVPARFSGSGSETEFTLTISSLQSEDFAVYYCHHYSNWPQAFGQGTRLDIK

SEQ ID NO: 200 - skipped

SEQ ID NO: 201 - DNA, 391 bp, synthetic construct
  source 1..391: mol_type = other DNA; note = MPK171-v1 VH (nt)
caggtgcagctggtgcagtctggagcagaggtgaaaaagcccggggagtctctgaagatctcctgtaagggttctggatacagctttattaattactggatcggctgggtgcgccagatgcccgggaaaggcctggagtggatgggaatcatctatccttttgactctgataccaaatacagcccgtccttccaaggccacgtcaccatctcagccgacaagtccatcaacaccgcctacgtgcagtggagcagcctgaaggcctcggacaccgccatgtattactgtgcgagatttacctttggtattgcagtggtcacagatgatgttagtggggaaggccatgcttttgatatctggggccaagggacaatggtcaccgtctcttcag

SEQ ID NO: 202 - protein, 130 aa, synthetic construct
  source 1..130: note = MPK171-v1 VH (aa)
QVQLVQSGAEVKKPGESLKISCKGSGYSFINYWIGWVRQMPGKGLEWMGIIYPFDSDTKYSPSFQGHVTISADKSINTAYVQWSSLKASDTAMYYCARFTFGIAVVTDDVSGEGHAFDIWGQGTMVTVSS

SEQ ID NO: 203 - protein, 23 aa, synthetic construct
  source 1..23: note = MPK171-v1; MPK171-v2 CDR-H3 (aa)
ARFTFGIAVVTDDVSGEGHAFDI

SEQ ID NO: 204 - DNA, 331 bp, synthetic construct
  source 1..331: mol_type = other DNA; note = MPK171-v1 VL (nt)
cagtctgtgctgactcagccgccctcagtgtctggggccccagggcagagggtcaccatctcctgcgccgggagcagctccaacatcggggcaggttttgatgtccactggtaccagcaagttccaggaacagcccccaaacttctcatctataataccgacaatcgggcctcaggggtccctgaccgattctctgcctccaagtctggcacctcagcctccctggccatcactgggctccaggctgaggatgaggctgattattattgccagtcctatgacaacagcctgagtggggtgttcggcggagggaccaagctgaccgtcctac

SEQ ID NO: 205 - protein, 110 aa, synthetic construct
  source 1..110: note = MPK171-v1 VL (aa)
QSVLTQPPSVSGAPGQRVTISCAGSSSNIGAGFDVHWYQQVPGTAPKLLIYNTDNRASGVPDRFSASKSGTSASLAITGLQAEDEADYYCQSYDNSLSGVFGGGTKLTVL

SEQ ID NO: 206 - protein, 9 aa, synthetic construct
  source 1..9: note = MPK171-v1; MPK182 CDR-L1 (aa)
SSNIGAGFD

SEQ ID NO: 207 - skipped

SEQ ID NO: 208 - protein, 10 aa, synthetic construct
  source 1..10: note = MPK171-v1; MPK182 CDR-L3 (aa)
QSYDNSLSGV

SEQ ID NO: 209 - DNA, 391 bp, synthetic construct; identical to SEQ ID NO: 201
  source 1..391: mol_type = other DNA; note = MPK171-v2 VH (nt)

SEQ ID NO: 210 - protein, 130 aa, synthetic construct; identical to SEQ ID NO: 202
  source 1..130: note = MPK171-v2 VH (aa)

SEQ ID NO: 211 - DNA, 322 bp, synthetic construct
  source 1..322: mol_type = other DNA; note = MPK171-v2 VL (nt)
cagtctgtgttgacgcagtctccagccaccctgtctgtgtctccaggggaaagagccaccctctcctgcagggccagtcagagtgttgccggcaacttagcctggtaccagcagagacctggccaggctcccaggctcctcatttatggtgcatccaccagggccactggtgtcccagccaggttcagtggcagtgggtctgagacagagttcactctcaccatcagcagcctgcagtctgaagattttgcagtttattactgtcagcagtatagtaactggcctcaagccttcggccaagggacacgactggacattaaac

SEQ ID NO: 212 - protein, 107 aa, synthetic construct
  source 1..107: note = MPK171-v2 VL (aa)
QSVLTQSPATLSVSPGERATLSCRASQSVAGNLAWYQQRPGQAPRLLIYGASTRATGVPARFSGSGSETEFTLTISSLQSEDFAVYYCQQYSNWPQAFGQGTRLDIK

SEQ ID NO: 213 - protein, 9 aa, synthetic construct
  source 1..9: note = MPK171-v2; MPK189; MPK195; MPK198 CDR-L3 (aa)
QQYSNWPQA

SEQ ID NO: 214 - DNA, 367 bp, synthetic construct
  source 1..367: mol_type = other DNA; note = MPK173 VH (nt)
caggtccagcttgtgcagtctggagctgaggtgaggaagcctggggcctcagtgaaggtctcctgcagggcctctggttacaccttttccaactttggtatcacttgggtgcgacaggcccctggacaagggcttgagtggatggggtggatcagcgcttataatggcaacacaaagagtgcacagaagttccaggacagagtcaccatgaccacagacacaaccacgagcacagcctacatggagctgaggagcctgagatctgacgacacggccgtctattactgtgcgagagacactccaagttcagacggagctgccctccttgactactggggccagggaaccctggtcaccgtctcctcac

SEQ ID NO: 215 - protein, 122 aa, synthetic construct
  source 1..122: note = MPK173 VH (aa)
QVQLVQSGAEVRKPGASVKVSCRASGYTFSNFGITWVRQAPGQGLEWMGWISAYNGNTKSAQKFQDRVTMTTDTTTSTAYMELRSLRSDDTAVYYCARDTPSSDGAALLDYWGQGTLVTVSS

SEQ ID NO: 216 - protein, 8 aa, synthetic construct
  source 1..8: note = MPK173 CDR-H1 (aa)
GYTFSNFG

SEQ ID NO: 217 - protein, 8 aa, synthetic construct
  source 1..8: note = MPK173 CDR-H2 (aa)
ISAYNGNT

SEQ ID NO: 218 - protein, 15 aa, synthetic construct
  source 1..15: note = MPK173 CDR-H3 (aa)
ARDTPSSDGAALLDY

SEQ ID NO: 219 - DNA, 334 bp, synthetic construct
  source 1..334: mol_type = other DNA; note = MPK173 VL (nt)
gatattgtgatgacccagactccactctccctgcccgtcacccttggacagccggcctccatctcctgcaggtctagtcaaagtctcgtatacagtgatggaaacacctacttgagttggtttcaacagaggccaggccaatctccaaggcgcctaatttataaggtttctaaccgggactctggggtcccggacagattcagcggcagtggctcaggcacttatttcacactgaaaatcaacagggtggaggctgaagatgttggggtttattattgcatgcagagtaaatactggctgacgttcggccaagggaccaaggtggaaatcaaac

SEQ ID NO: 220 - protein, 111 aa, synthetic construct
  source 1..111: note = MPK173 VL (aa)
DIVMTQTPLSLPVTLGQPASISCRSSQSLVYSDGNTYLSWFQQRPGQSPRRLIYKVSNRDSGVPDRFSGSGSGTYFTLKINRVEAEDVGVYYCMQSKYWLTFGQGTKVEIK

SEQ ID NO: 221 - protein, 11 aa, synthetic construct
  source 1..11: note = MPK173; MPM8 CDR-L1 (aa)
QSLVYSDGNTY

SEQ ID NO: 222 - skipped

SEQ ID NO: 223 - protein, 8 aa, synthetic construct
  source 1..8: note = MPK173 CDR-L3 (aa)
MQSKYWLT

SEQ ID NO: 224 - DNA, 391 bp, synthetic construct
  source 1..391: mol_type = other DNA; note = MPK175 VH (nt)
caggtgcagcttgtgcagtctggagcagaggtgaaaaagcccggggagtctctgaagatctcctgtcaggcttctggatacagctttattaattactggatcggctgggtgcgccagaagcccgggaaaggcctggagtggatgggaatcatctatccttttgactctgagaccaaatacagcccgtccttccaaggccacgtcaccatctcagccgacagattcaccaataccgcctacgtgcagtggaccagcctgaaggcctcggacaccgccatctattactgtgcgagatttacctttggtcttgcagtggtcgcaggtggtcttagtgacgacggccaagcttttgatatctggggccaagggacaagggtcaccgtctcttcag

SEQ ID NO: 225 - protein, 130 aa, synthetic construct
  source 1..130: note = MPK175 VH (aa)
QVQLVQSGAEVKKPGESLKISCQASGYSFINYWIGWVRQKPGKGLEWMGIIYPFDSETKYSPSFQGHVTISADRFTNTAYVQWTSLKASDTAIYYCARFTFGLAVVAGGLSDDGQAFDIWGQGTRVTVSS

SEQ ID NO: 226 - protein, 8 aa, synthetic construct
  source 1..8: note = MPK175; MPK186; MPK201; MPK201 VH.4 CDR-H2 (aa)
IYPFDSET

SEQ ID NO: 227 - protein, 23 aa, synthetic construct
  source 1..23: note = MPK175 CDR-H3 (aa)
ARFTFGLAVVAGGLSDDGQAFDI

SEQ ID NO: 228 - DNA, 322 bp, synthetic construct
  source 1..322: mol_type = other DNA; note = MPK175 VL (nt)
gaaattgtgttgacgcagtctccagccaccctgtctgtgtctccaggggaaagagccaccctctcctgcagggccagtcagagtgttgccgccaacctagcctggtaccagcagagacctggccagactcccaggctcctcatttatggtgcatccaccagggccactggtgtcccagccaggttcagtggcagtgggtctgagacagagttcactctcaccatcagcagcctgcagcctgaagattttgcagtttattactgtcaccactatagtgactggcctcaagccttcggccaagggacacgacttgacattaaac

SEQ ID NO: 229 - protein, 107 aa, synthetic construct
  source 1..107: note = MPK175 VL (aa)
EIVLTQSPATLSVSPGERATLSCRASQSVAANLAWYQQRPGQTPRLLIYGASTRATGVPARFSGSGSETEFTLTISSLQPEDFAVYYCHHYSDWPQAFGQGTRLDIK

SEQ ID NO: 230 - protein, 6 aa, synthetic construct
  source 1..6: note = MPK175 CDR-L1 (aa)
QSVAAN

SEQ ID NO: 231 - protein, 9 aa, synthetic construct
  source 1..9: note = MPK175; MPK201 CDR-L3 (aa)
HHYSDWPQA

SEQ ID NO: 232 - DNA, 370 bp, synthetic construct
  source 1..370: mol_type = other DNA; note = MPK176 VH.1; MPK176 VH (nt)
caggtgcagctggtgcagtctggagctgaggtgaagaagccaggggcctcagtgagggtctcctgcaaggcttcaggttacacccttaccgcctacggtatcagctgggtgcgacaggcccctggacaaggccttgagtggatggggtggatcagccctaagcatgggaacacacactatgcacagaggttccagggcagagtcaccatgaccacagacacgtccaccagtacagcctacatggaactgaggagcctgagacctgacgacaccgccgtttatttctgtgcgagagagggcgtggatataatggcggttccctggtattttgactactggggccagggaaccctggtcgccgtctcctcag

SEQ ID NO: 233 - protein, 123 aa, synthetic construct
  source 1..123: note = MPK176 VH.1; MPK176 VH (aa)
QVQLVQSGAEVKKPGASVRVSCKASGYTLTAYGISWVRQAPGQGLEWMGWISPKHGNTHYAQRFQGRVTMTTDTSTSTAYMELRSLRPDDTAVYFCAREGVDIMAVPWYFDYWGQGTLVAVSS

SEQ ID NO: 234 - protein, 8 aa, synthetic construct
  source 1..8: note = MPK176 VH.1; MPK176 VH.2; MPK176 VH.3; MPK176 VH.4; MPK176 VH.5; MPK176 VH.6; MPK176 CDR-H1 (aa)
GYTLTAYG

SEQ ID NO: 235 - protein, 8 aa, synthetic construct
  source 1..8: note = MPK176 VH.1; MPK176 VH.2; MPK176 VH.3; MPK176 VH.5; MPK176 CDR-H2 (aa)
ISPKHGNT

SEQ ID NO: 236 - protein, 16 aa, synthetic construct
  source 1..16: note = MPK176 VH.1; MPK176; MPK176 VH.4 CDR-H3 (aa)
AREGVDIMAVPWYFDY

SEQ ID NO: 237 - DNA, 325 bp, synthetic construct
  source 1..325: mol_type = other DNA; note = MPK176 VL.1; MPK176 VL (nt)
tcctatgagctgacacagccaccctcggtgtcagtggccccaggacagacggccaggatttcctgtgggggaaacaacattggaagtaaaagtgtgcactggtaccaacagaagccaggccaggcccctgtggtggtcgtctatgatgatagcgaccggccctcagggatccctgagcgattctctggctccaactctgggaacacggccaccctgaccatcagcggggtcgaagccggggatgaggccgactatttctgccaggtgtgggatagtgctactgatcattgggtgttcggcggagggaccaagttgaccgtcctag

SEQ ID NO: 238 - protein, 108 aa, synthetic construct
  source 1..108: note = MPK176 VL.1; MPK176 VL (aa)
SYELTQPPSVSVAPGQTARISCGGNNIGSKSVHWYQQKPGQAPVVVVYDDSDRPSGIPERFSGSNSGNTATLTISGVEAGDEADYFCQVWDSATDHWVFGGGTKLTVL

SEQ ID NO: 239 - protein, 6 aa, synthetic construct
  source 1..6: note = MPK176 VL.1; MPK176 VL.2; MPK176 VL.3; MPK176 VL.4; MPK176 VL.5; MPK176 CDR-L1 (aa)
NIGSKS

SEQ ID NO: 240 - skipped

SEQ ID NO: 241 - protein, 11 aa, synthetic construct
  source 1..11: note = MPK176 VL.1; MPK176 VL.2; MPK176 VL.3; MPK176 VL.4; MPK176 VL.5; MPK176 CDR-L3 (aa)
QVWDSATDHWV

SEQ ID NO: 242 - DNA, 391 bp, synthetic construct
  source 1..391: mol_type = other DNA; note = MPK177 VH (nt)
gaggtgcagctggtgcagtctggagcagaggtgaaaaagcccggggagtctctgaagatctcctgtcaggtttctggatacagctttatgaattattgggtcggctgggtgcgccagatgcccgggaaaggcctggagtggatgggaatcatctatccttttgactctgataccatatacagtccgtccttccaaggacacgtcaccatctcagccgacacgtccatcacgaccgcctacctacattggagcagcctgaaggcctcggacaccgccacctattactgtgcgagatttaccttcgggattgcggtggtcgcagatggtcttagtggggatggagaagcttttgatatctggggccaaggaacagtggtcaccgtctcttcag

SEQ ID NO: 243 - protein, 130 aa, synthetic construct
  source 1..130: note = MPK177 VH (aa)
EVQLVQSGAEVKKPGESLKISCQVSGYSFMNYWVGWVRQMPGKGLEWMGIIYPFDSDTIYSPSFQGHVTISADTSITTAYLHWSSLKASDTATYYCARFTFGIAVVADGLSGDGEAFDIWGQGTVVTVSS

SEQ ID NO: 244 - protein, 8 aa, synthetic construct
  source 1..8: note = MPK177 CDR-H1 (aa)
GYSFMNYW

SEQ ID NO: 245 - protein, 23 aa, synthetic construct
  source 1..23: note = MPK177 CDR-H3 (aa)
ARFTFGIAVVADGLSGDGEAFDI

SEQ ID NO: 246 - DNA, 322 bp, synthetic construct
  source 1..322: mol_type = other DNA; note = MPK177 VL (nt)
gatattgtgatgacccagactccagccaccctgtctgtgtctccaggggaaagagccaccctctcctgcagggccagtcagagtgttgccggcaagttagcctggtaccagcagagacctggccagactcccaggctcctcatttatggtgcatccaccagggccactggtgtcccagccaggttcagtggcagtgggtctgagacagagttcactctcaccatcagcagcctgcagtctgaagattttgcagtttattactgtcagcagtatagttcctggcctcacgccttcggccaggggacacgactggacattaaac

SEQ ID NO: 247 - protein, 107 aa, synthetic construct
  source 1..107: note = MPK177 VL (aa)
DIVMTQTPATLSVSPGERATLSCRASQSVAGKLAWYQQRPGQTPRLLIYGASTRATGVPARFSGSGSETEFTLTISSLQSEDFAVYYCQQYSSWPHAFGQGTRLDIK

SEQ ID NO: 248 - protein, 9 aa, synthetic construct
  source 1..9: note = MPK177 CDR-L3 (aa)
QQYSSWPHA

SEQ ID NO: 249 - DNA, 391 bp, synthetic construct
  source 1..391: mol_type = other DNA; note = MPK178 VH (nt)
gaggtgcagctggtgcagtctggagcagaggtgaaaaagcccggggagtctctgaagatctcctgtagggcctctggatacagctttgttaattattggatcggctgggtgcgccaggtgcccgggaaaggcctggagtggatgggattcatctatccttttgactctgatacccaatacagtccgtccttccaaggccacgtcaccatctcagccgacaagtccatcaacaccgcctatttgcagtggagcagcctgagggcctcggacaccgccatttattactgtgcgagatttacctttggtattgcattggtcgcaggtgatgttactgggggaggccaggcttttgaagtctggggccaagggacgttggtcagcgtgtcttcag

SEQ ID NO: 250 - protein, 130 aa, synthetic construct
  source 1..130: note = MPK178 VH (aa)
EVQLVQSGAEVKKPGESLKISCRASGYSFVNYWIGWVRQVPGKGLEWMGFIYPFDSDTQYSPSFQGHVTISADKSINTAYLQWSSLRASDTAIYYCARFTFGIALVAGDVTGGGQAFEVWGQGTLVSVSS

SEQ ID NO: 251 - DNA, 322 bp, synthetic construct
  source 1..322: mol_type = other DNA; note = MPK178 VL (nt)
gatattgtgatgactcagtctccagtcaccctgtctgtctctccaggggaaagagccaccctctcttgcagggccagtcagagtgttgccggcaacttagcctggtatcaacagagacctggccaggctcccaggctcctcatttatgctgcatccaccagggccactggtgtcccagccaggttcagtggcagtgggtctgagacagagttcactctcaccatcagcagcctgcagtctgaagattttgcagtttattactgtcaccactatagtaactggcctcaagccttcggccaagggacacgactagacattaaac

SEQ ID NO: 252 - protein, 107 aa, synthetic construct
  source 1..107: note = MPK178 VL (aa)
DIVMTQSPVTLSVSPGERATLSCRASQSVAGNLAWYQQRPGQAPRLLIYAASTRATGVPARFSGSGSETEFTLTISSLQSEDFAVYYCHHYSNWPQAFGQGTRLDIK

SEQ ID NO: 253 - DNA, 391 bp, synthetic construct
  source 1..391: mol_type = other DNA; note = MPK179-v4 VH (nt)
caggtccagctggtgcagtctagagcagaggtgaagaagcccggggagtctctgaagatctcctgtagggcttctggatacagctttattaattactggatcggctgggtgcgccagatgcccgggaaaggcctggagtggatgggaatcatctatccttttgactctgataccaaatatagtccgtcctttgaaggccacgtcaccatctcagccgacaagtccatcaacaccgcctacgtgcagtggagcagcctgaaggcctcggacaccgccatttattactgtgcgagatttacctttggaattgctctggtcgcaggtgatgttggtggggaaggccatgcttttgatatctggggccaagggacactggtcaccgtctcctcag

SEQ ID NO: 254 - protein, 130 aa, synthetic construct
  source 1..130: note = MPK179-v4 VH (aa)
QVQLVQSRAEVKKPGESLKISCRASGYSFINYWIGWVRQMPGKGLEWMGIIYPFDSDTKYSPSFEGHVTISADKSINTAYVQWSSLKASDTAIYYCARFTFGIALVAGDVGGEGHAFDIWGQGTLVTVSS

SEQ ID NO: 255 - protein, 23 aa, synthetic construct
  source 1..23: note = MPK179-v4 CDR-H3 (aa)
ARFTFGIALVAGDVGGEGHAFDI

SEQ ID NO: 256 - DNA, 325 bp, synthetic construct
  source 1..325: mol_type = other DNA; note = MPK179-v4 VL (nt)
gatattgtgatgactcagtctccttcctccctgtctgcatctgttggcgacagagtcaccatcacttgccgggcaagtcagacaattagcacctatttaaattggtatcagcagaagccagggaaagcccctaaactcctgatctatgctgcatccagattgcaaagtggtgtcccatcacgattcagtggcagtggatctgggacagatttcactctcaccatcagcagtctgcaacctgaagattttgcaacttacttctgtcaacagagttacaatgcccctccattcaccttcggccctgggaccaaagtggatatcagac

SEQ ID NO: 257 - protein, 108 aa, synthetic construct
  source 1..108: note = MPK179-v4 VL (aa)
DIVMTQSPSSLSASVGDRVTITCRASQTISTYLNWYQQKPGKAPKLLIYAASRLQSGVPSRFSGSGSGTDFTLTISSLQPEDFATYFCQQSYNAPPFTFGPGTKVDIR

SEQ ID NO: 258 - protein, 6 aa, synthetic construct
  source 1..6: note = MPK179-v4 CDR-L1 (aa)
QTISTY

SEQ ID NO: 259 - protein, 10 aa, synthetic construct
  source 1..10: note = MPK179-v4 CDR-L3 (aa)
QQSYNAPPFT

SEQ ID NO: 260 - DNA, 355 bp, synthetic construct
  source 1..355: mol_type = other DNA; note = MPK180 VH (nt)
caggtccagctggtgcagtctggagcagaggtgaagaagcccggggagtctctgaggatttcctgtaaggcttcaggatacagttttgccagctactggatcggctgggtgcgccagaagcccggaaaaggcccagagtggatggggatcatctttcctaatgactctgataccgaatacagcccgtccttcgaaggccaggtcagcatttcagccgacaagtccaccagcaccgtccatttgcagtggggcagcctgaaggcctcggacaccgccgtgtattactgtgggaggatgtattatttaggggcccttgaaacctggggccaggggacaatggtcaccgtctcttcag

SEQ ID NO: 261 - protein, 118 aa, synthetic construct
  source 1..118: note = MPK180 VH (aa)
QVQLVQSGAEVKKPGESLRISCKASGYSFASYWIGWVRQKPGKGPEWMGIIFPNDSDTEYSPSFEGQVSISADKSTSTVHLQWGSLKASDTAVYYCGRMYYLGALETWGQGTMVTVSS

SEQ ID NO: 262 - protein, 8 aa, synthetic construct
  source 1..8: note = MPK180 CDR-H1 (aa)
GYSFASYW

SEQ ID NO: 263 - protein, 8 aa, synthetic construct
  source 1..8: note = MPK180 CDR-H2 (aa)
IFPNDSDT

SEQ ID NO: 264 - protein, 11 aa, synthetic construct
  source 1..11: note = MPK180 CDR-H3 (aa)
GRMYYLGALET

SEQ ID NO: 265 - DNA, 322 bp, synthetic construct
  source 1..322: mol_type = other DNA; note = MPK180 VL (nt)
gccatccggatgacccagtctccatcctccctgtctgcatctgtcggagacagagtcaccatagtttgtcaggcgagtcacgacattagcaactttttaaattggtatcagcagagaccagggcaagcccctaagctcctgatctacgatgcatcctttttggaaacaggggtcccatctaggttcagtggaagtggatctgggacatattttactctcaccatcagcagcctgcagcctgaagattttgcaacatattactgtcagcggtatgataatcgcttcttcactttcggccctgggaccaaagtggacatcaaac

SEQ ID NO: 266 - protein, 107 aa, synthetic construct
  source 1..107: note = MPK180 VL (aa)
AIRMTQSPSSLSASVGDRVTIVCQASHDISNFLNWYQQRPGQAPKLLIYDASFLETGVPSRFSGSGSGTYFTLTISSLQPEDFATYYCQRYDNRFFTFGPGTKVDIK

SEQ ID NO: 267 - protein, 6 aa, synthetic construct
  source 1..6: note = MPK180 CDR-L1 (aa)
HDISNF

SEQ ID NO: 268 - skipped

SEQ ID NO: 269 - protein, 9 aa, synthetic construct
  source 1..9: note = MPK180 CDR-L3 (aa)
QRYDNRFFT

SEQ ID NO: 270 - DNA, 391 bp, synthetic construct
  source 1..391: mol_type = other DNA; note = MPK181 VH (nt)
gaggtgcagctggtgcagtctggagcagaggtgaaaaagcccggggagtctctgaagatctcctgtcaggtttctggatacagctttattaattattggatcggctgggtgcgccagatgcccgggaaaggcctggagtggatgggaatcatctatccttttgactctgataccaaatacagcccgtccttccaaggacacgtcaccatctcagccgacacgtccatcaggaccgcctacctgcagtggagcagcctgaaggcctcggacaccgccatgtatttctgtgcgagatttaccttcggtattgcagtggtcgcagatggtcttagcggggaagggcaagcttttgatctctggggccaagggacagtggtcaccgtctcctcag

SEQ ID NO: 271 - protein, 130 aa, synthetic construct
  source 1..130: note = MPK181 VH (aa)
EVQLVQSGAEVKKPGESLKISCQVSGYSFINYWIGWVRQMPGKGLEWMGIIYPFDSDTKYSPSFQGHVTISADTSIRTAYLQWSSLKASDTAMYFCARFTFGIAVVADGLSGEGQAFDLWGQGTVVTVSS

SEQ ID NO: 272 - protein, 23 aa, synthetic construct
  source 1..23: note = MPK181 CDR-H3 (aa)
ARFTFGIAVVADGLSGEGQAFDL

SEQ ID NO: 273 - DNA, 322 bp, synthetic construct
  source 1..322: mol_type = other DNA; note = MPK181 VL (nt)
gaaatagtgatgacgcagtctccagccaccctgtctgtgtctccaggggaaagagccaccctctcctgcagggccagtcagagtgttgccggcaagttagcctggtaccagcagagacctggccagactcccaggctcctcatttatggtgcatccaccagggccactggtgtcccagccaggttcagtggcagtgggtctgagacagagttcactctcaccatcagcagcctgcagtctgaagattttgcagtttattactgtcagcagtatactacctggcctcacgccttcggccaggggacacgactggacattaaac

SEQ ID NO: 274 - protein, 107 aa, synthetic construct
  source 1..107: note = MPK181 VL (aa)
EIVMTQSPATLSVSPGERATLSCRASQSVAGKLAWYQQRPGQTPRLLIYGASTRATGVPARFSGSGSETEFTLTISSLQSEDFAVYYCQQYTTWPHAFGQGTRLDIK

SEQ ID NO: 275 - protein, 9 aa, synthetic construct
  source 1..9: note = MPK181 CDR-L3 (aa)
QQYTTWPHA

SEQ ID NO: 276 - DNA, 367 bp, synthetic construct
  source 1..367: mol_type = other DNA; note = MPK182 VH (nt)
gaggtgcagctggtggagtccgggggagggctggtcaagcctggcgggtccctgagactctcctgcacagcctctttaagtaccttcagtgactactacatgacctggatccgccaggctcctgggaaggggctggagtggcttgggtacatcagtggtagtcagagtcagacaaatctcgcagactctgtgaagggtcgcttcaccatctccagagacaacgccaagaagtcgctctatctgcagatgaacagcctgagagtcgaagacacggctacatatttttgtgcgagagtccacctgtggttcggagaccttaatgctcttgatctctggggccaaggaacaaaggtcaccgtctcttcag

SEQ ID NO: 277 - protein, 122 aa, synthetic construct
  source 1..122: note = MPK182 VH (aa)
EVQLVESGGGLVKPGGSLRLSCTASLSTFSDYYMTWIRQAPGKGLEWLGYISGSQSQTNLADSVKGRFTISRDNAKKSLYLQMNSLRVEDTATYFCARVHLWFGDLNALDLWGQGTKVTVSS

SEQ ID NO: 278 - protein, 8 aa, synthetic construct
  source 1..8: note = MPK182 CDR-H1 (aa)
LSTFSDYY

SEQ ID NO: 279 - protein, 8 aa, synthetic construct
  source 1..8: note = MPK182 CDR-H2 (aa)
ISGSQSQT

SEQ ID NO: 280 - protein, 15 aa, synthetic construct
  source 1..15: note = MPK182 CDR-H3 (aa)
ARVHLWFGDLNALDL

SEQ ID NO: 281 - DNA, 331 bp, synthetic construct
  source 1..331: mol_type = other DNA; note = MPK182 VL (nt)
cagtctgtgctgacgcagccgccctcagtgtctggggccccagggcagagggtcaccatctcctgcgccgggagcagctccaacatcggggcaggttttgatgtccactggtaccagcaagttccaggaacagcccccaaacttctcatctataataccgacaatcgggcctcaggggtccctgaccgattctctgcctccaagtctggcacttcagccttcctggccatcactgggctccaggctgaggatgaggctgattattattgccagtcctatgacaacagcctgagtggggtgttcggcggagggaccaagctgaccgtcctac

SEQ ID NO: 282 - protein, 110 aa, synthetic construct
  source 1..110: note = MPK182 VL (aa)
QSVLTQPPSVSGAPGQRVTISCAGSSSNIGAGFDVHWYQQVPGTAPKLLIYNTDNRASGVPDRFSASKSGTSAFLAITGLQAEDEADYYCQSYDNSLSGVFGGGTKLTVL

SEQ ID NO: 283 - DNA, 387 bp, synthetic construct
  source 1..387: mol_type = other DNA; note = MPK185 VH (nt)
gaggtgcagctggtgcagtctggagcagaggtgaagaagcccggggagtctctcaagatctcctgtacgggtcctgaatacaccttcagtaacttctggatcgcctgggtgcgccagatgcccgggaaaggcctggagtggttgggtttcatttatccttttgactctgataccaaatacaacccgtccttccaaggccaggtcatcatgtcagccgacaagtccctcaacaccgcctacctacagtgggccagcctgaaggcctcggacaccgccatttattactgtgcgagactcttcgctggctggttgtatacaactagtgacgcaccaaaaactcacgctatggacgtctggggccaagggaccagggtcaccgtctcttta

SEQ ID NO: 284 - protein, 129 aa, synthetic construct
  source 1..129: note = MPK185 VH (aa)
EVQLVQSGAEVKKPGESLKISCTGPEYTFSNFWIAWVRQMPGKGLEWLGFIYPFDSDTKYNPSFQGQVIMSADKSLNTAYLQWASLKASDTAIYYCARLFAGWLYTTSDAPKTHAMDVWGQGTRVTVSL

SEQ ID NO: 285 - protein, 8 aa, synthetic construct
  source 1..8: note = MPK185 CDR-H1 (aa)
EYTFSNFW

SEQ ID NO: 286 - protein, 22 aa, synthetic construct
  source 1..22: note = MPK185 CDR-H3 (aa)
ARLFAGWLYTTSDAPKTHAMDV

SEQ ID NO: 287 - DNA, 331 bp, synthetic construct
  source 1..331: mol_type = other DNA; note = MPK185 VL (nt)
cagactgtggtgactcagcctgcctccgtgtctgggtctcctggacagtcgatcaccttctcctgcactggaaccagcagtgacgttggtgctcatgactatgtctcctggtaccaacaccacccaggcaaagcccccaaactcttgatttatgaggtcactaatcggccctcaggagtttctaatcgcttctctggctccaagtctgccaacacggcctccctgaccatctctgggctccaggctgacgacgaggctgattattactgcagctcatatgcaaccagtaacacttgggtgttcggcggcgggaccaagttgaccgtcctaa

SEQ ID NO: 288 - protein, 110 aa, synthetic construct
  source 1..110: note = MPK185 VL (aa)
QTVVTQPASVSGSPGQSITFSCTGTSSDVGAHDYVSWYQHHPGKAPKLLIYEVTNRPSGVSNRFSGSKSANTASLTISGLQADDEADYYCSSYATSNTWVFGGGTKLTVL

SEQ ID NO: 289 - protein, 9 aa, synthetic construct
  source 1..9: note = MPK185 CDR-L1 (aa)
SSDVGAHDY

SEQ ID NO: 290 - skipped

SEQ ID NO: 291 - protein, 10 aa, synthetic construct
  source 1..10: note = MPK185 CDR-L3 (aa)
SSYATSNTWV

SEQ ID NO: 292 - DNA, 391 bp, synthetic construct
  source 1..391: mol_type = other DNA; note = MPK186 VH (nt)
caggtccagcttgtgcagtctggagcagaggtgaaaaagcccggggagtctctgaagatctcctgtcagggttctggatacagctttattaattactggatcggctgggtgcgccagatgcccgggaaaggcctggagtggatgggaatcatctatccttttgactctgaaaccaaatacagcccgtccttccaaggccacgtcaccatctcagccgacaagtccatcaacaccgcctacgtgcagtggagcagcctgaaggcctcggacaccgccatgtattactgtgcgagatttacctttggtattgcagtggtcacagatgatgtttatggggaagcccaaggctttgatatctggggccaagggacaatggtcaccgtctcttcag

SEQ ID NO: 293 - protein, 130 aa, synthetic construct
  source 1..130: note = MPK186 VH (aa)
QVQLVQSGAEVKKPGESLKISCQGSGYSFINYWIGWVRQMPGKGLEWMGIIYPFDSETKYSPSFQGHVTISADKSINTAYVQWSSLKASDTAMYYCARFTFGIAVVTDDVYGEAQGFDIWGQGTMVTVSS

SEQ ID NO: 294 - protein, 23 aa, synthetic construct
  source 1..23: note = MPK186 CDR-H3 (aa)
ARFTFGIAVVTDDVYGEAQGFDI

SEQ ID NO: 295 - DNA, 322 bp, synthetic construct
  source 1..322: mol_type = other DNA; note = MPK186 VL (nt)
gatattgtgatgacccagactccagccaccctgtctgtgtctccaggggaaagagccaccctctcctgcagggccagtcagagtgttgacggcaacttagcctggtaccagcagagacctggccaggctcccaggctcctcatttatggtgcatcgaccagggccactggtgtcccagccaggttcagtggcagtgggtctgagacagagttcactctcaccatcagcagcctgcagtctgaagattttgcagtttattactgtcagcactatagtaactggcctcaagccttcggccaagggacacgactggacattaaac

SEQ ID NO: 296 - protein, 107 aa, synthetic construct
  source 1..107: note = MPK186 VL (aa)
DIVMTQTPATLSVSPGERATLSCRASQSVDGNLAWYQQRPGQAPRLLIYGASTRATGVPARFSGSGSETEFTLTISSLQSEDFAVYYCQHYSNWPQAFGQGTRLDIK

SEQ ID NO: 297 - protein, 6 aa, synthetic construct
  source 1..6: note = MPK186 CDR-L1 (aa)
QSVDGN

SEQ ID NO: 298 - protein, 9 aa, synthetic construct
  source 1..9: note = MPK186 CDR-L3 (aa)
QHYSNWPQA

SEQ ID NO: 299 - DNA, 358 bp, synthetic construct
  source 1..358: mol_type = other DNA; note = MPK187 VH (nt)
caggtccagctggtgcagtccggggctgaggtgaagaagcctggggcctcagtgaaggtctcctgcaagacttctggatactacttcaacgactactttattcactgggtgcgacacgcccctggacaagggcttgagtggatgggatgggtcaaccctttgagtggtgccacagtttatgcacagaaatttcagggctgggtcaccttgacccgggacacatccatcaacactgcctacatggagctgactagactgacatccaatgacacggccatgtattactgtgcgaccggggcccttttttggactggtccgacttactggggccagggaaccccggtcaccgtctctgcag

SEQ ID NO: 300 - protein, 119 aa, synthetic construct
  source 1..119: note = MPK187 VH (aa)
QVQLVQSGAEVKKPGASVKVSCKTSGYYFNDYFIHWVRHAPGQGLEWMGWVNPLSGATVYAQKFQGWVTLTRDTSINTAYMELTRLTSNDTAMYYCATGALFWTGPTYWGQGTPVTVSA

SEQ ID NO: 301 - protein, 8 aa, synthetic construct
  source 1..8: note = MPK187; MPK197 CDR-H1 (aa)
GYYFNDYF

SEQ ID NO: 302 - protein, 8 aa, synthetic construct
  source 1..8: note = MPK187; MPK197 CDR-H2 (aa)
VNPLSGAT

SEQ ID NO: 303 - protein, 12 aa, synthetic construct
  source 1..12: note = MPK187; MPK197 CDR-H3 (aa)
ATGALFWTGPTY

SEQ ID NO: 304 - DNA, 328 bp, synthetic construct
  source 1..328: mol_type = other DNA; note = MPK187 VL (nt)
gaaattgtgttgacacagtctccagccaccctgtctttgtttccaggggaaagagccaccctctcctgcaggaccagtcagtttgttagtaataacttagcctggtaccaacagagacctggccaggctcccaggctcctcatctatgatgcatccagtcgggccagtggcaccccaggcaggttcagtgccagtgggtctgggacagacttcactctcaccgtcagcagcctagagactgaagactttgcaatttattactgtcaacaccgaagcaactggcctccggggatcactttcggccctgggaccagagtggatattagac

SEQ ID NO: 305 - protein, 109 aa, synthetic construct
  source 1..109: note = MPK187 VL (aa)
EIVLTQSPATLSLFPGERATLSCRTSQFVSNNLAWYQQRPGQAPRLLIYDASSRASGTPGRFSASGSGTDFTLTVSSLETEDFAIYYCQHRSNWPPGITFGPGTRVDIR

SEQ ID NO: 306 - protein, 6 aa, synthetic construct
  source 1..6: note = MPK187 CDR-L1 (aa)
QFVSNN

SEQ ID NO: 307 - protein, 11 aa, synthetic construct
  source 1..11: note = MPK187; MPK197 CDR-L3 (aa)
QHRSNWPPGIT

SEQ ID NO: 308 - DNA, 391 bp, synthetic construct
  source 1..391: mol_type = other DNA; note = MPK188 VH (nt)
gaggtgcagctggtgcagtctggagcagaggtgaaaaagcccggggagtctctgaagatctcctgtcaggcttctggatacagctttattaattactggatcggctgggtgcgccagatgcccgggaaaggcctggagtggatgggcatcatctatccttttgactctgataccaactacagcccgtccttccaaggcctcgtcaccatctcagccgacaagtccatcaacaccgcctacctgcagtggcgcagcctgaaggcctcggacaccgccatgtattactgtgcgcgatttacctttggtattgcactggtcgcaggtgatgttaatggggaaggccatgcttttgatatctggggccaagggacagtggtcaccgtctcttcag

SEQ ID NO: 309 - protein, 130 aa, synthetic construct
  source 1..130: note = MPK188 VH (aa)
EVQLVQSGAEVKKPGESLKISCQASGYSFINYWIGWVRQMPGKGLEWMGIIYPFDSDTNYSPSFQGLVTISADKSINTAYLQWRSLKASDTAMYYCARFTFGIALVAGDVNGEGHAFDIWGQGTVVTVSS

SEQ ID NO: 310 - protein, 23 aa, synthetic construct
  source 1..23: note = MPK188 CDR-H3 (aa)
ARFTFGIALVAGDVNGEGHAFDI

SEQ ID NO: 311 - DNA, 322 bp, synthetic construct
  source 1..322: mol_type = other DNA; note = MPK188 VL (nt)
gaaattgtgatgacgcagtctccagccaccctgtctgtgtctccaggggaaagtgccaccctctcctgcagggccagtcagggtgttgccggcaacttagcctggtaccagcagagacctggccaggctcccaggctcctcatttatggtacatccaccagggccaccggtgtcccagccaggttcagtggcagtgggtctgagacagagttcactctcaccatcagcagcctgcagtctgaagattttgcagtttattactgtcaccactatagtaactggcctcaagccttcggccaagggacacgactggacattaaac

SEQ ID NO: 312 - protein, 107 aa, synthetic construct
  source 1..107: note = MPK188 VL (aa)
EIVMTQSPATLSVSPGESATLSCRASQGVAGNLAWYQQRPGQAPRLLIYGTSTRATGVPARFSGSGSETEFTLTISSLQSEDFAVYYCHHYSNWPQAFGQGTRLDIK

SEQ ID NO: 313 - protein, 6 aa, synthetic construct
  source 1..6: note = MPK188 CDR-L1 (aa)
QGVAGN

SEQ ID NO: 314 - skipped

SEQ ID NO: 315 - DNA, 391 bp, synthetic construct
  source 1..391: mol_type = other DNA; note = MPK189 VH (nt)
caggtgcagctggtgcagtctggagcagaggtgaaaaagcccggggagtctctgaagatctcctgtaagggttctggatacagctttattaattactggatcggctgggtgcgccagatgcccgggaaaggcctggaatggatgggaatcatctatccttttgactctgataccaaatacagcccgtccttccaaggccacgtcaccatctcagccgacaagtccatcaacaccgcctacgtgcagtggagcagcctgaaggcctcggacaccgccatgtattactgtgcgagatttacctttggtattgcagtggtcacagatgatgttagtgacgagggccatgcttttgatatctggggccaagggacaatggtcaccgtctcctcag

SEQ ID NO: 316 - protein, 130 aa, synthetic construct
  source 1..130: note = MPK189 VH (aa)
QVQLVQSGAEVKKPGESLKISCKGSGYSFINYWIGWVRQMPGKGLEWMGIIYPFDSDTKYSPSFQGHVTISADKSINTAYVQWSSLKASDTAMYYCARFTFGIAVVTDDVSDEGHAFDIWGQGTMVTVSS

SEQ ID NO: 317 - protein, 23 aa, synthetic construct
  source 1..23: note = MPK189 CDR-H3 (aa)
ARFTFGIAVVTDDVSDEGHAFDI

SEQ ID NO: 318 - DNA, 322 bp, synthetic construct
  source 1..322: mol_type = other DNA; note = MPK189 VL (nt)
gaaatagtgatgatgcagtctccagccaccctgtctgtgtctccaggggaaagagccaccctctcctgcagggccagtcagagtgttgccggcaacttagcctggtaccagcagagacctggccaggctcccaggctcctcatttatggtgcatccaccagggccactggtgtcccagccaggttcagtggcagtgggtctgagacagagttcactctcaccatcagcagcctgcagtctgaagattttgcagtttattactgtcagcagtatagtaactggcctcaagccttcggccaagggacacgactggacattaaac

SEQ ID NO: 319 - protein, 107 aa, synthetic construct
  source 1..107: note = MPK189 VL (aa)
EIVMMQSPATLSVSPGERATLSCRASQSVAGNLAWYQQRPGQAPRLLIYGASTRATGVPARFSGSGSETEFTLTISSLQSEDFAVYYCQQYSNWPQAFGQGTRLDIK

SEQ ID NO: 320 - DNA, 391 bp, synthetic construct
  source 1..391: mol_type = other DNA; note = MPK191 VH (nt)
gaggtgcagctggtgcagtctggagcagaggtgaaaaagcccggggagtctctgaagatctcctgtaagggttctggatacagttttattaattactggatcggctgggtgcgccagatgcccgggaaaggcctggagtggatgggaatcatctatccttttgactctgataccaaatacagcccgtccttccaaggccacgtcaccatctcagccgacaagtccatcaataccgcctacgtgcagtggagcggcctgaaggcctcggacaccgccatgtattattgtgcgagatttacctttggaattgcagtggtcgcaggtgatgtcactggggaaggccatgcttttgatgtctggggccaagggacaatggtcaccgtctcttcag

SEQ ID NO: 321 - protein, 130 aa, synthetic construct
  source 1..130: note = MPK191 VH (aa)
EVQLVQSGAEVKKPGESLKISCKGSGYSFINYWIGWVRQMPGKGLEWMGIIYPFDSDTKYSPSFQGHVTISADKSINTAYVQWSGLKASDTAMYYCARFTFGIAVVAGDVTGEGHAFDVWGQGTMVTVSS

SEQ ID NO: 322 - protein, 23 aa, Synthetic construct
  source 1..23: note = MPK191 CDR-H3 (aa)
ARFTFGIAVVAGDVTGEGHAFDV

SEQ ID NO: 323 - DNA, 322 bp, synthetic construct
  source 1..322: mol_type = other DNA; note = MPK191 VL (nt)
gaaatagtgatgacgcagtctccagccaccctgtctgtgtctccaggggatagagccaccctctcctgcagggccagtcagagtgtggccggcaacttagcctggtaccagcagagacctggccagactcccaggctcctcatttatggtgcatccaccagggcctctggtgtcccagccaggttcagtggcagtgggtctgagacagagttcactctcaccatcagcagcctgcagtctgaagattttgcagtttattactgtcagcagtatgataactggcctcacgccttcggccaagggacacgactggacattaaac

SEQ ID NO: 324 - protein, 107 aa, synthetic construct
  source 1..107: note = MPK191 VL (aa)
EIVMTQSPATLSVSPGDRATLSCRASQSVAGNLAWYQQRPGQTPRLLIYGASTRASGVPARFSGSGSETEFTLTISSLQSEDFAVYYCQQYDNWPHAFGQGTRLDIK

SEQ ID NO: 325 - protein, 9 aa, synthetic construct
  source 1..9: note = MPK191 CDR-L3 (aa)
QQYDNWPHA

SEQ ID NO: 326 - DNA, 391 bp, synthetic construct
  source 1..391: mol_type = other DNA; note = MPK193 VH (nt)
gaggtgcagctggtgcagtctggagcagaggtgaaaaagcccggggagtctctgaagatctcctgtgaggcttctggatacagctttaataattactggatcggctgggtgcgccagatgcccgggaaaggcctggagtggatgggaatcatctatccttttgactcggataccaaatacagcccgtccttccaaggccacgtcaccatctcagccgacaagtccatcaacaccgcctacctgcagtggagcagtttgaaggcctcggacaccgccatgtacttctgtgcgagatttacttttggtattgtcttggtcgcaggtgatgttactggggaaggccatgcttttgatgtctggggccgagggaccatggtcaccgtcacttcag

SEQ ID NO: 327 - protein, 130 aa, synthetic construct
  source 1..130: note = MPK193 VH (aa)
EVQLVQSGAEVKKPGESLKISCEASGYSFNNYWIGWVRQMPGKGLEWMGIIYPFDSDTKYSPSFQGHVTISADKSINTAYLQWSSLKASDTAMYFCARFTFGIVLVAGDVTGEGHAFDVWGRGTMVTVTS

SEQ ID NO: 328 - protein, 23 aa, synthetic construct
  source 1..23: note = MPK193 CDR-H3 (aa)
ARFTFGIVLVAGDVTGEGHAFDV

SEQ ID NO: 329 - DNA, 322 bp, synthetic construct
  source 1..322: mol_type = other DNA; note = MPK193 VL (nt)
gaaatagtgatgatgcagtctccagtcaccctgtctgtgtctccagggggaagagccaccctctcctgcagggccagtcagagtgttgccggcaacttagcctggtaccaacagagacctggccaggctcccaggctcctcatttatggtgcatccaccagggccactggtgtcccagccaggttcagtggcagtgggtctgagacagagttcactctcaccatcagcagcctgcagtctgaagattttgcagtttattactgtcatcattatagtaactggcctcaggccttcggccaagggacacgactggacattaaac

SEQ ID NO: 330 - protein, 107 aa, synthetic construct
  source 1..107: note = MPK193 VL (aa)
EIVMMQSPVTLSVSPGGRATLSCRASQSVAGNLAWYQQRPGQAPRLLIYGASTRATGVPARFSGSGSETEFTLTISSLQSEDFAVYYCHHYSNWPQAFGQGTRLDIK

SEQ ID NO: 331 - DNA, 364 bp, synthetic construct
  source 1..364: mol_type = other DNA; note = MPK194-v2 VH (nt)
caggtgcagctggtggaatctgggggaggcgtggtccagcctgggaggtccctgagactctcctgtgtagcctctggattcaccttcagtaattatcctctccactgggtccgccaggctccaggcaaggggctacagtgggtggcagttatatcatatgatggaagtgagaactactacgctgactccgtgaagggccgattcaccatctccagagacaattccaagagcacactgtatttgcaaatgaacagcctgagacctgaggacacggctatctattactgtgcgagaagtaaggtggctggggggagctattactttgactactggggccagggaaccctggtccccgtctcctcag

SEQ ID NO: 332 - protein, 121 aa, synthetic construct
  source 1..121: note = MPK194-v2 VH (aa)
QVQLVESGGGVVQPGRSLRLSCVASGFTFSNYPLHWVRQAPGKGLQWVAVISYDGSENYYADSVKGRFTISRDNSKSTLYLQMNSLRPEDTAIYYCARSKVAGGSYYFDYWGQGTLVPVSS

SEQ ID NO: 333 - protein, 8 aa, synthetic construct
  source 1..8: note = MPK194-v2 CDR-H1 (aa)
GFTFSNYP

SEQ ID NO: 334 - protein, 8 aa, synthetic construct
  source 1..8: note = MPK194-v2 CDR-H2 (aa)
ISYDGSEN

SEQ ID NO: 335 - protein, 14 aa, synthetic construct
  source 1..14: note = MPK194-v2 CDR-H3 (aa)
ARSKVAGGSYYFDY

SEQ ID NO: 336 - DNA, 325 bp, synthetic construct
  source 1..325: mol_type = other DNA; note = MPK194-v2 VL (nt)
tcctatgagctgactcagccaccctcggtgtcagtggccccaggacaggcggccagggtttcctgtgggggaaacaacattggaagtcaatatgtacagtggtaccagcagaagccgggccaggcccctgtcctggtcatctatgatgacgacgaccggccctcagggatccctgagcgattctctggctccaactctgggagcacggccaccctgaccatcagcagggtcgaagccggggatgaggccgactattactgtcaggtgtgggataaaagtaatcatgttgtggtattcggcggagggaccaagttgaccgtcctag

SEQ ID NO: 337 - protein, 108 aa, synthetic construct
  source 1..108: note = MPK194-v2 VL (aa)
SYELTQPPSVSVAPGQAARVSCGGNNIGSQYVQWYQQKPGQAPVLVIYDDDDRPSGIPERFSGSNSGSTATLTISRVEAGDEADYYCQVWDKSNHVVVFGGGTKLTVL

SEQ ID NO: 338 - protein, 6 aa, synthetic construct
  source 1..6: note = MPK194-v2 CDR-L1 (aa)
NIGSQY

SEQ ID NO: 339 - skipped

SEQ ID NO: 340 - protein, 11 aa, synthetic construct
  source 1..11: note = MPK194-v2 CDR-L3 (aa)
QVWDKSNHVVV

SEQ ID NO: 341 - DNA, 391 bp, synthetic construct
  source 1..391: mol_type = other DNA; note = MPK195 VH (nt)
gaggtgcaactggtgcagtctggagcagcggtgaaaaagcccggggagtctttgaagatctcctgtaggggttctggatacagctttatcaattactggatcggctgggtgcgccagatgcccggtaaaggcctggagtggatgggagtcatctatccttttgactctgatacccaatacagtccgtccttccaaggccacgtcaccatctcagccgacaagtccatcaacaccgcctacctgcagtgggacagcctgaaggccgcggacaccgccatatattactgtgcgagatttacctttggtattgcattatccactgctgatgtttacggggaagggcaagctttcgatatatggggcccagggacaatggtcaccgtctcttcag

SEQ ID NO: 342 - protein, 130 aa, synthetic construct
  source 1..130: note = MPK195 VH (aa)
EVQLVQSGAAVKKPGESLKISCRGSGYSFINYWIGWVRQMPGKGLEWMGVIYPFDSDTQYSPSFQGHVTISADKSINTAYLQWDSLKAADTAIYYCARFTFGIALSTADVYGEGQAFDIWGPGTMVTVSS

SEQ ID NO: 343 - protein, 23 aa, synthetic construct
  source 1..23: note = MPK195 CDR-H3 (aa)
ARFTFGIALSTADVYGEGQAFDI

SEQ ID NO: 344 - DNA, 322 bp, synthetic construct
  source 1..322: mol_type = other DNA; note = MPK195 VL (nt)
gaaattgtgttgacacagtctccagccaccctgtctgtgtctccaggggaaagagccaccctctcctgcagggccagtcagagtgttgccggcaagatagcctggtaccagcagagacctggccaggctcccaggctcctcatttatggtgcatccaccagggccactggtgtcccagccaggttcagtggcagtgggtctgagacagagttcactctcaccatcagcagcctgcagtctgaggattttgcagtttattactgtcaacaatatagtaactggcctcaagccttcggcccagggacacggctggacatgaaac

SEQ ID NO: 345 - protein, 107 aa, synthetic construct
  source 1..107: note = MPK195 VL (aa)
EIVLTQSPATLSVSPGERATLSCRASQSVAGKIAWYQQRPGQAPRLLIYGASTRATGVPARFSGSGSETEFTLTISSLQSEDFAVYYCQQYSNWPQAFGPGTRLDMK

SEQ ID NO: 346 - DNA, 358 bp, synthetic construct
  source 1..358: mol_type = other DNA; note = MPK197 VH (nt)
caggtgcagctggtgcagtccggggctgaggtgaagaagcctggggcctcagtgaaggtctcctgcaagacttctggatactacttcaacgactactttattcactgggtgcgacacgcccctggccaagggcttgagtggatgggatgggtcaaccctttgagtggtgccacagtttatgcacagaagtttcagggctgggtcaccttgaccagggacacgtccatcaacactgcctacatggaattgactagactgacatccaacgacacggccctatattactgtgcgaccggggccctcttttggactggtccaacatactggggccagggaaccccggtcaccgtctctgcag

SEQ ID NO: 347 - protein, 119 aa, synthetic construct
  source 1..119: note = MPK197 VH (aa)
QVQLVQSGAEVKKPGASVKVSCKTSGYYFNDYFIHWVRHAPGQGLEWMGWVNPLSGATVYAQKFQGWVTLTRDTSINTAYMELTRLTSNDTALYYCATGALFWTGPTYWGQGTPVTVSA

SEQ ID NO: 348 - DNA, 328 bp, synthetic construct
  source 1..328: mol_type = other DNA; note = MPK197 VL (nt)
gaaatagtgatgatgcagtctccagccaccctgtctttgtctccaggggaaagagccaccctctcctgcagggccagtcagagtattagtaataacttagcctggtaccaacagaaaactggccaggctcccaggctcctcatctatgatgcatccagtcgggccagtggcaccccaggcaggttcagtgccagtgggtctgggacagacttcactctcaccgtcagcagcctagagactgaagattttgcaatttattactgtcaacatcgcagcaattggcctccggggatcactttcggccctgggaccagagtggatgttagac

SEQ ID NO: 349 - protein, 109 aa, synthetic construct
  source 1..109: note = MPK197 VL (aa)
EIVMMQSPATLSLSPGERATLSCRASQSISNNLAWYQQKTGQAPRLLIYDASSRASGTPGRFSASGSGTDFTLTVSSLETEDFAIYYCQHRSNWPPGITFGPGTRVDVR

SEQ ID NO: 350 - protein, 6 aa, synthetic construct
  source 1..6: note = MPK197 CDR-L1 (aa)
QSISNN

SEQ ID NO: 351 - DNA, 391 bp, synthetic construct
  source 1..391: mol_type = other DNA; note = MPK198 VH (nt)
gaggtgcagctggtgcagtctggagcagaggtgaaaaagcccggggagtctctgaagatctcctgtaagggttctggatacagctttattaattactggatcggctgggtgcgccagatgcccgggaaaggcctggagtggatgggaatcatctatccttttgactctgataccaaatacagcccgtccttccaaggccacgtcaccatctcagccgacaagtccatcaacaccgcctacgtgcagtggagcagcctgaaggcctcggacaccgccatgtattactgtgcgagatttacctttggtattgcagtggtcacagatgatgttagtggggaaggccaagcttttgatatctggggccaagggacaatggtcaccgtctcttcag

SEQ ID NO: 352 - protein, 130 aa, synthetic construct
  source 1..130: note = MPK198 VH (aa)
EVQLVQSGAEVKKPGESLKISCKGSGYSFINYWIGWVRQMPGKGLEWMGIIYPFDSDTKYSPSFQGHVTISADKSINTAYVQWSSLKASDTAMYYCARFTFGIAVVTDDVSGEGQAFDIWGQGTMVTVSS

SEQ ID NO: 353 - protein, 23 aa, synthetic construct
  source 1..23: note = MPK198 CDR-H3 (aa)
ARFTFGIAVVTDDVSGEGQAFDI

SEQ ID NO: 354 - DNA, 322 bp, synthetic construct; identical to SEQ ID NO: 318
  source 1..322: mol_type = other DNA; note = MPK198 VL (nt)

SEQ ID NO: 355 - protein, 107 aa, synthetic construct; identical to SEQ ID NO: 319
  source 1..107: note = MPK198 VL (aa)

SEQ ID NO: 356 - DNA, 391 bp, synthetic construct
  source 1..391: mol_type = other DNA; note = MPK201 VH.1; MPK201 VH (nt)
gaggtgcagctggtgcagtctggagcagaggtgaaaaagcccggggagtctctgaagatctcctgtcagggttctggatacagctttattaattactggatcggctgggtgcgccagaagcccgggaaaggcctggagtggatgggaatcatctatccttttgactctgagaccaaatacagcccgtccttccaaggccacgtcaccttctcagccgacaagtccatcaacaccgcctacgtgcagtggagcagcctgagggcctcggacaccgccatgtattactgtgcgagatttacctttggtcttgcagtgaccactgctgatgttagtggggaaggccaggcttttgaaatctggggccaagggacaatggtcaccgtctcttcag

SEQ ID NO: 357 - protein, 130 aa, synthetic construct
  source 1..130: note = MPK201 VH.1; MPK201 VH (aa)
EVQLVQSGAEVKKPGESLKISCQGSGYSFINYWIGWVRQKPGKGLEWMGIIYPFDSETKYSPSFQGHVTFSADKSINTAYVQWSSLRASDTAMYYCARFTFGLAVTTADVSGEGQAFEIWGQGTMVTVSS

SEQ ID NO: 358 - protein, 23 aa, synthetic construct
  source 1..23: note = MPK201 VH.1; MPK201 VH.2; MPK201 VH.3; MPK201 VH.4; MPK201 VH.5; MPK201 VH.6; MPK201 CDR-H3 (aa)
ARFTFGLAVTTADVSGEGQAFEI

SEQ ID NO: 359 - DNA, 322 bp, synthetic construct
  source 1..322: mol_type = other DNA; note = MPK201 VK.1; MPK201 VL (nt)
gaaatagtgatgatgcagtctccagccaccctgtctgtgtctccaggggaaagagccaccctctcctgcagggccagtcagagtgttgccggcaacttagcctggtaccagcagagacctggccaggctcccagactcctcgtttatggtgcatccaccagggccagtggtgtcccagccaggttcagtggcagtgggtctgagacagagttcactctcaccatcagcagcctgcagtctgaagattttgcagtttattactgtcaccattatagtgattggcctcaagccttcggccaagggacacgactggacattaaac

SEQ ID NO: 360 - protein, 107 aa, synthetic construct
  source 1..107: note = MPK201 VK.1; MPK201 VL (aa)
EIVMMQSPATLSVSPGERATLSCRASQSVAGNLAWYQQRPGQAPRLLVYGASTRASGVPARFSGSGSETEFTLTISSLQSEDFAVYYCHHYSDWPQAFGQGTRLDIK

SEQ ID NO: 361 - DNA, 376 bp, synthetic construct
  source 1..376: mol_type = other DNA; note = MPK202 VH (nt)
gaggtgcagctggtacagtcaggggctgaggtgaagaagactggggcctcagtgaaggtctcctgcaaggttttcggagacaccctcactgaactatccatgcactgggtgcgacaggctcctggaaaagggcttgagtggatgggaggttttcatcgtgaagagggtgaaacaatctacgcacagaagtttcagggcagagtcaccatgaccgaggacacatcttcagacacagcctacatggagctgagcagcctgagatctgacgacacggccatgtattactgtgcaacattagtggatatagtggtgacgaccgccggagcaggagccaaccactactggggccagggaaccatggtcaccgtctcctcag

SEQ ID NO: 362 - protein, 125 aa, synthetic construct
  source 1..125: note = MPK202 VH (aa)
EVQLVQSGAEVKKTGASVKVSCKVFGDTLTELSMHWVRQAPGKGLEWMGGFHREEGETIYAQKFQGRVTMTEDTSSDTAYMELSSLRSDDTAMYYCATLVDIVVTTAGAGANHYWGQGTMVTVSS

SEQ ID NO: 363 - protein, 8 aa, synthetic construct
  source 1..8: note = MPK202 CDR-H1 (aa)
GDTLTELS

SEQ ID NO: 364 - protein, 8 aa, synthetic construct
  source 1..8: note = MPK202 CDR-H2 (aa)
FHREEGET

SEQ ID NO: 365 - protein, 18 aa, synthetic construct
  source 1..18: note = MPK202 CDR-H3 (aa)
ATLVDIVVTTAGAGANHY

SEQ ID NO: 366 - DNA, 337 bp, synthetic construct
  source 1..337: mol_type = other DNA; note = MPK202 VL (nt)
gatattgtgatgactcagtctccactctccctgcccgtcacccctggagagccggcctccatctcctgcaggtctagtcagagcctcctgcatagtaatggatacaactatttggattggtacctgcagaagccagggcagtctccacaggtcctgatctatttgggttctaatcgggcctccggggtccctgacaggttcagtggcagtggatcaggcacagattttacactgaaaatcagcagagtggaggctgaggatgttggggtgtattactgcatgcaaggtctacaaactcccttcactttcggcggagggaccaaggtggagatcaaac

SEQ ID NO: 367 - protein, 112 aa, synthetic construct
  source 1..112: note = MPK202 VL (aa)
DIVMTQSPLSLPVTPGEPASISCRSSQSLLHSNGYNYLDWYLQKPGQSPQVLIYLGSNRASGVPDRFSGSGSGTDFTLKISRVEAEDVGVYYCMQGLQTPFTFGGGTKVEIK

SEQ ID NO: 368 - DNA, 382 bp, synthetic construct
  source 1..382: mol_type = other DNA; note = MPK203 VH (nt)
gaggtgcagctggtgcaatctgggcctcaggtgaaaaagcctggggccgcggtgaaggtctcctgcaaggcttctggcttcaccttcactgatactactgtcaactgggtgcgacagacccgtggacaaaggcttgagtggatgggatggatcgccgttggcagtgggaccactaactacgcacagaagttccgggaccgagtcaccatttcccgcgacacgtccacagacacagcctacatggagctgggtggtctgacccacgatgacacggccgtctattactgtacgacagaggctagtcgtacttggttcgtccaaacaagcccctttgtgggtttcgacccctggggccagggaacgctggtcaccgtctcctcag

SEQ ID NO: 369 - protein, 127 aa, synthetic construct
  source 1..127: note = MPK203 VH (aa)
EVQLVQSGPQVKKPGAAVKVSCKASGFTFTDTTVNWVRQTRGQRLEWMGWIAVGSGTTNYAQKFRDRVTISRDTSTDTAYMELGGLTHDDTAVYYCTTEASRTWFVQTSPFVGFDPWGQGTLVTVSS

SEQ ID NO: 370 - protein, 8 aa, synthetic construct
  source 1..8: note = MPK203 CDR-H1 (aa)
GFTFTDTT

SEQ ID NO: 371 - protein, 8 aa, synthetic construct
  source 1..8: note = MPK203 CDR-H2 (aa)
IAVGSGTT

SEQ ID NO: 372 - protein, 20 aa, synthetic construct
  source 1..20: note = MPK203 CDR-H3 (aa)
TTEASRTWFVQTSPFVGFDP

SEQ ID NO: 373 - DNA, 322 bp, synthetic construct
  source 1..322: mol_type = other DNA; note = MPK203 VL (nt)
gaaattgtgttgacacagtctccaggcaccctgtctttgtctccaggggagagggccactctctcctgcagggccagtcagagtgttagcagcagctacttcgcctggtatcagcaaaaacctggccagactcccagggtcctcatatatggtgcatccaatagggcccctgacatcccagacaggttcagtggcagtgggtctgggacagacttcattctcaccatcagcagactggaacctgaagattttgcaatttattactgtcagcactatggttcctcaccgacgttcggccaagggaccaaggtggagatcaaac

SEQ ID NO: 374 - protein, 107 aa, synthetic construct
  source 1..107: note = MPK203 VL (aa)
EIVLTQSPGTLSLSPGERATLSCRASQSVSSSYFAWYQQKPGQTPRVLIYGASNRAPDIPDRFSGSGSGTDFILTISRLEPEDFAIYYCQHYGSSPTFGQGTKVEIK

SEQ ID NO: 375 - protein, 7 aa, synthetic construct
  source 1..7: note = MPK203 CDR-L1 (aa)
QSVSSSY

SEQ ID NO: 376 - protein, 8 aa, synthetic construct
  source 1..8: note = MPK203 CDR-L3 (aa)
QHYGSSPT

SEQ ID NO: 377 - DNA, 361 bp, synthetic construct
  source 1..361: mol_type = other DNA; note = MPK126 VH (nt)
gaggtgcagctggtggagtctgggggaggcttggtcaagcctggagggtccatgagactctcctgtgcagcctctggagtcaccctcagtggctactacatgatctggatccgccaggctccagggaaggggctggagtggatctcagacatcagcgccagtagtacttacacagactacgctgactctgtgaagggccgattcaccgtctccagagacaactcccagaacacggtttatctccaaatgaacagcctgagagccgaggacacggctgtgtattattgtgcgacagaacaaacaccgtcacctggcccctttgacctgtggggccagggaaccctggtcaccgtctcctcag

SEQ ID NO: 378 - protein, 120 aa, synthetic construct
  source 1..120: note = MPK126 VH (aa)
EVQLVESGGGLVKPGGSMRLSCAASGVTLSGYYMIWIRQAPGKGLEWISDISASSTYTDYADSVKGRFTVSRDNSQNTVYLQMNSLRAEDTAVYYCATEQTPSPGPFDLWGQGTLVTVSS

SEQ ID NO: 379 - skipped

SEQ ID NO: 380 - protein, 8 aa, synthetic construct
  source 1..8: note = MPK126 CDR-H1 (aa)
GVTLSGYY

SEQ ID NO: 381 - DNA, 334 bp, synthetic construct
  source 1..334: mol_type = other DNA; note = MPK126 VL (nt)
cagtctgtgctgacgcagccgccctcagtgtctggggccccagggcagagggtcaccatctcctgcactgggagcagctccaacgtcggggcaggttatgatgtacactggtaccaacagtttccaggaacatcccccaaactcctcatctatcgtaacaccaatcgaccctccggggtccctgaccgattctctggcttcaagtctggcacctcagcctccctggccatcactgggctcgaggctgaagatgaggctgattattactgccagtcctatgacagaagcctgagtggatgggttttcggcgcagggaccaagctgaccgtcctag

SEQ ID NO: 382 - protein, 111 aa, synthetic construct
  source 1..111: note = MPK126 VL (aa)
QSVLTQPPSVSGAPGQRVTISCTGSSSNVGAGYDVHWYQQFPGTSPKLLIYRNTNRPSGVPDRFSGFKSGTSASLAITGLEAEDEADYYCQSYDRSLSGWVFGAGTKLTVL

SEQ ID NO: 383 - protein, 9 aa, synthetic construct
  source 1..9: note = MPK126 CDR-L1 (aa)
SSNVGAGYD

SEQ ID NO: 384 - protein, 11 aa, synthetic construct
  source 1..11: note = MPK126; MPK146; MPK151; MPK157 CDR-L3 (aa)
QSYDRSLSGWV

SEQ ID NO: 385 - DNA, 361 bp, synthetic construct
  source 1..361: mol_type = other DNA; note = MPK127 VH (nt)
caggtgcagctggtgatctctgggggaggcctggtcaagccgggggggtccctgagactctcatgtgcagcctctggtttcagaatcgacagttttagtttgaactgggtccgccaggccccagggaagggactggagtggctcgcgtccgtgagtgccagtagtagttacatagactacagagactcagtggcggggcgattcaccatctccagagacaatgcccagaattcactgtatcttcaaatggacaccctgagagccgaggacacggctgtctattactgtgcgagaactaatggctacgaaacgggttcctttgatgtctggggccaagggactctggtcaccgtcgcttcag

SEQ ID NO: 386 - protein, 120 aa, synthetic construct
  source 1..120: note = MPK127 VH (aa)
QVQLVISGGGLVKPGGSLRLSCAASGFRIDSFSLNWVRQAPGKGLEWLASVSASSSYIDYRDSVAGRFTISRDNAQNSLYLQMDTLRAEDTAVYYCARTNGYETGSFDVWGQGTLVTVAS

SEQ ID NO: 387 - protein, 8 aa, synthetic construct
  source 1..8: note = MPK127 CDR-H1 (aa)
GFRIDSFS

SEQ ID NO: 388 - protein, 8 aa, synthetic construct
  source 1..8: note = MPK127 CDR-H2 (aa)
VSASSSYI

SEQ ID NO: 389 - protein, 13 aa, synthetic construct
  source 1..13: note = MPK127 CDR-H3 (aa)
ARTNGYETGSFDV

SEQ ID NO: 390 - DNA, 334 bp, synthetic construct
  source 1..334: mol_type = other DNA; note = MPK127 VL (nt)
cagtctgtgctgacgcagccgccctcagtgtctggggccccagggcagagggtcaccatctcctgcactgggagcagctccaacatcggggcaggttatgatgtccactggtaccagcaacttccaggaaaagcccccaaactcctcatcaacgctaacaacaatcggccctcaggggtccctgaccgattctctggctccaactctggcacctcagcctccctggccatcactggcctccaggctgaggatgaggctgattattactgccagtcctatgacaaaaccctgagtggttatgtcttcggacttgggacaacggtcaccgtcctgg

SEQ ID NO: 391 - protein, 111 aa, synthetic construct
  source 1..111: note = MPK127 VL (aa)
QSVLTQPPSVSGAPGQRVTISCTGSSSNIGAGYDVHWYQQLPGKAPKLLINANNNRPSGVPDRFSGSNSGTSASLAITGLQAEDEADYYCQSYDKTLSGYVFGLGTTVTVL

SEQ ID NO: 392 - skipped

SEQ ID NO: 393 - protein, 11 aa, synthetic construct
  source 1..11: note = MPK127 CDR-L3 (aa)
QSYDKTLSGYV

SEQ ID NO: 394 - DNA, 361 bp, synthetic construct
  source 1..361: mol_type = other DNA; note = MPK129 VH (nt)
gaggtgcagctggtggagtctgggggaggcttggtcaagcctggagggtccctaagactctcctgtgaagcctctggattctccctcagtggctactacatgatctggatccgccaggctccagggaaggggctcgagtggatttcatccattagtggaagtggtaactacatagactacgctgattctgtgaagggccgattcaccatctccagagacaacggccacaacacactgtatctccagatgaacagcctgagagtcgaggacacggctgtctactattgtgcgacagaacaaacgccatcccctggcccctttgaccactggggccagggaaccctggtcaccgtctcctctg

SEQ ID NO: 395 - protein, 120 aa, synthetic construct
  source 1..120: note = MPK129 VH (aa)
EVQLVESGGGLVKPGGSLRLSCEASGFSLSGYYMIWIRQAPGKGLEWISSISGSGNYIDYADSVKGRFTISRDNGHNTLYLQMNSLRVEDTAVYYCATEQTPSPGPFDHWGQGTLVTVSS

SEQ ID NO: 396 - protein, 8 aa, synthetic construct
  source 1..8: note = MPK129; MPK136 CDR-H1 (aa)
GFSLSGYY

SEQ ID NO: 397 - protein, 8 aa, synthetic construct
  source 1..8: note = MPK129; MPK136 CDR-H2 (aa)
ISGSGNYI

SEQ ID NO: 398 - protein, 13 aa, synthetic construct
  source 1..13: note = MPK129; MPK136 CDR-H3 (aa)
ATEQTPSPGPFDH

SEQ ID NO: 399 - DNA, 334 bp, synthetic construct
  source 1..334: mol_type = other DNA; note = MPK129 VL (nt)
cagactgtggtgacgcagccgccctcagtgtctggggccccagggcagagggtcaccatctcctgcactgggaccagctccaacatcgggacaggttatgatgtacactggtaccaacagtttccaggaacatcccccaaactcctcatctatcgtaataccaatcggccctcaggggtccctgaccgattctctggctccaagtctggctcctcagcctccctggccatcgctgggctccaggctgaggatgaggctgattattactgccagtcgtatgacagaagtgtgagtgggtgggttttcggcggagggaccaagctgaccgtcctag

SEQ ID NO: 400 - protein, 111 aa, synthetic construct
  source 1..111: note = MPK129 VL (aa)
QTVVTQPPSVSGAPGQRVTISCTGTSSNIGTGYDVHWYQQFPGTSPKLLIYRNTNRPSGVPDRFSGSKSGSSASLAIAGLQAEDEADYYCQSYDRSVSGWVFGGGTKLTVL

SEQ ID NO: 401 - protein, 9 aa, synthetic construct
  source 1..9: note = MPK129; MPK132-v1; MPK136; MPK141; MPK150-v2; MPK204 CDR-L1 (aa)
SSNIGTGYD

SEQ ID NO: 402 - protein, 11 aa, synthetic construct
  source 1..11: note = MPK129; MPK132-v1; MPK136; MPK149; MPK155; MPK204 CDR-L3 (aa)
QSYDRSVSGWV

SEQ ID NO: 403 - DNA, 361 bp, synthetic construct
  source 1..361: mol_type = other DNA; note = MPK130 VH (nt)
gaggtgcagctggtggagtcggggggaggcttggtcaagcctggagggtccctgacactctcctgtgaagcctctggattcagtataagtggctactacatgatctgggtccgccaggctccagggaagggcctggagtgggtctcctccattagtgccagtgatacttatatagactacgcagtctctgtgcagggccgattcaccatctccagacacaacaccaagaactcgctttatctgcagatgagcagcctgagagtcgaggacacgggtgtctattattgtacgagagaaaaggtgccgtcgcctggcccctttgactgctggggccagggaaccctggtcaccgtctcctcag

SEQ ID NO: 404 - protein, 120 aa, synthetic construct
  source 1..120: note = MPK130 VH (aa)
EVQLVESGGGLVKPGGSLTLSCEASGFSISGYYMIWVRQAPGKGLEWVSSISASDTYIDYAVSVQGRFTISRHNTKNSLYLQMSSLRVEDTGVYYCTREKVPSPGPFDCWGQGTLVTVSS

SEQ ID NO: 405 - protein, 8 aa, synthetic construct
  source 1..8: note = MPK130 CDR-H2 (aa)
ISASDTYI

SEQ ID NO: 406 - protein, 13 aa, synthetic construct
  source 1..13: note = MPK130 CDR-H3 (aa)
TREKVPSPGPFDC

SEQ ID NO: 407 - DNA, 334 bp, synthetic construct
  source 1..334: mol_type = other DNA; note = MPK130 VL (nt)
cagtctgtgttgacgcagccgccctcagtgtctggggccccagggcagaggatcaccatctcctgcactgggacgagctccaacatcggggcgggttatgatgtacactggtaccagcaccttccaggagcagcccccagactcctcatctatgttaacaccaatcgaccctcaggggtccctgaccgattctctggcgcccggtcaggcacctcagcctccctggccatcactgggctccaggctgaggatgaggctgattattactgccagtcctacgacagaagtctgggtggttgggcgttcggcgaagggaccaaggtgaccgtcctag

SEQ ID NO: 408 - protein, 111 aa, synthetic construct
  source 1..111: note = MPK130 VL (aa)
QSVLTQPPSVSGAPGQRITISCTGTSSNIGAGYDVHWYQHLPGAAPRLLIYVNTNRPSGVPDRFSGARSGTSASLAITGLQAEDEADYYCQSYDRSLGGWAFGEGTKVTVL

SEQ ID NO: 409 - skipped

SEQ ID NO: 410 - protein, 11 aa, synthetic construct
  source 1..11: note = MPK130 CDR-L3 (aa)
QSYDRSLGGWA

SEQ ID NO: 411 - DNA, 361 bp, synthetic construct
  source 1..361: mol_type = other DNA; note = MPK132-v1 VH (nt)
gaggtgcagctggtggagtctgggggaggcttggtcaagcctggagggtccctaagactctcctgtgaagcctctggaatctccctcagtgggtactacatgatctggatccgccaggctccagggaagggactggagtggatttcatccatcagtagtagtagtaattatattgactacgctgactctgtgaagggccgattcaccatctccagacacaacggccagaacacactctatctccagatgaacagcctgagagtcgaggacacggctgtttatttttgtgcgacagagcaaacgccgtcccctggcccctttgaattgtggggccagggaatcctggtcaccgtctcatcag

SEQ ID NO: 412 - protein, 120 aa, synthetic construct
  source 1..120: note = MPK132-v1 VH (aa)
EVQLVESGGGLVKPGGSLRLSCEASGISLSGYYMIWIRQAPGKGLEWISSISSSSNYIDYADSVKGRFTISRHNGQNTLYLQMNSLRVEDTAVYFCATEQTPSPGPFELWGQGILVTVSS

SEQ ID NO: 413 - DNA, 334 bp, synthetic construct
  source 1..334: mol_type = other DNA; note = MPK132-v1 VL (nt)
cagtctgtcgtgacgcagccgccctcagtgtctggggccccagggcagagggtcaccatcttctgcactgggaccagctccaacatcgggacaggttatgatgtacactggtaccaacagtttccaggaacatcccccaaactcctcatctatcgtaataccaatcggccctcaggggtccctgaccgattctctggctccaagtctggctcctcagcctccctggccatcgctgggctccaggctgaggatgaggctgattattactgccagtcgtatgacagaagtgtgagtggatgggttttcggcggagggaccaagctgaccgtcctag

SEQ ID NO: 414 - protein, 111 aa, synthetic construct
  source 1..111: note = MPK132-v1 VL (aa)
QSVVTQPPSVSGAPGQRVTIFCTGTSSNIGTGYDVHWYQQFPGTSPKLLIYRNTNRPSGVPDRFSGSKSGSSASLAIAGLQAEDEADYYCQSYDRSVSGWVFGGGTKLTVL

SEQ ID NO: 415 - DNA, 361 bp, synthetic construct
  source 1..361: mol_type = other DNA; note = MPK133 VH (nt)
caggtgcagctggtgcagtctgggggaggcttggtcaagcctggagggtccctgaggctctcctgtgcagcctctgaattcagcttcagtggttactttatgatctggatccgtcaggctccaggtaaggggctggagtggatttcatacatcagtggaagtagtacctacacaaattacgctgactctgtgaagggccgattcaccatttccagagacaacgcccagaacacactgtatctcgaaatgaacagcctgagagccgaagacacgggtgtctattattgtgcgacagaacaggtgccgtcacctggcccctttgacctctggggccagggaaccctggtcaccgtctcctcag

SEQ ID NO: 416 - protein, 120 aa, synthetic construct
  source 1..120: note = MPK133 VH (aa)
QVQLVQSGGGLVKPGGSLRLSCAASEFSFSGYFMIWIRQAPGKGLEWISYISGSSTYTNYADSVKGRFTISRDNAQNTLYLEMNSLRAEDTGVYYCATEQVPSPGPFDLWGQGTLVTVSS

SEQ ID NO: 417 - protein, 8 aa, synthetic construct
  source 1..8: note = MPK133 CDR-H1 (aa)
EFSFSGYF

SEQ ID NO: 418 - DNA, 334 bp, synthetic construct
  source 1..334: mol_type = other DNA; note = MPK133 VL (nt)
cagtctgtgttgacgcagccgccctcggtgtctggggccccagggcagagggtcaccatctcctgcactgggagcagctccaacatcggggcaggttatgatgtacactggtaccaacagtttccaggaagatcccccacactcctcatctatcgtaataccaatcggccctcaggggtccctgaccgattctctggctcccagtctggctcctcagcctccctggccatcactggactccaggctgaggatgaggctgattattactgccagtcctatgaccgaaccctgagtggatgggctttcggcggagggaccaaggtgaccgtcctag

SEQ ID NO: 419 - protein, 111 aa, synthetic construct
  source 1..111: note = MPK133 VL (aa)
QSVLTQPPSVSGAPGQRVTISCTGSSSNIGAGYDVHWYQQFPGRSPTLLIYRNTNRPSGVPDRFSGSQSGSSASLAITGLQAEDEADYYCQSYDRTLSGWAFGGGTKVTVL

SEQ ID NO: 420 - protein, 11 aa, synthetic construct
  source 1..11: note = MPK133 CDR-L3 (aa)
QSYDRTLSGWA

SEQ ID NO: 421 - DNA, 361 bp, synthetic construct
  source 1..361: mol_type = other DNA; note = MPK136 VH (nt)
gaggtgcagctggtggagtctgggggaggcttggtcaagcctggagggtccctaagactctcctgtgaagcctctggattctccctcagtggctactacatgatctggatccgccaggctccagggaaggggctcgagtggatttcatccattagtggaagtggtaactacataaactacgctgattctgtgaagggccgattcaccgtctccagagacaacggccacaacacactgtatctccagatgaacagcctgagagtcgaggacacggctgtctactattgtgcgacagaacaaacgccatcccctggcccctttgaccactggggccagggaaccctggtcaccgtctcctctg

SEQ ID NO: 422 - protein, 120 aa, synthetic construct
  source 1..120: note = MPK136 VH (aa)
EVQLVESGGGLVKPGGSLRLSCEASGFSLSGYYMIWIRQAPGKGLEWISSISGSGNYINYADSVKGRFTVSRDNGHNTLYLQMNSLRVEDTAVYYCATEQTPSPGPFDHWGQGTLVTVSS

SEQ ID NO: 423 - DNA, 334 bp, synthetic construct
  source 1..334: mol_type = other DNA; note = MPK136 VL (nt)
caggtccagctggtgcagccgccctcagtgtctggggccccagggcagagggtctccatctcctgcactgggaccagctccaacatcgggacaggttatgatgtacactggtatcaacaatttccaggaacatcccccaaactcctcatctatcgtaataccaatcggccctcaggggtccctgaccgattctctggctccaagtctggctcctcagcctccctggccatcgctgggctccaggctgaggatgaggctgattattactgccagtcgtatgacagaagtgtgagtgggtgggttttcggcggagggaccaagctgaccgtcctag

SEQ ID NO: 424 - protein, 111 aa, synthetic construct
  source 1..111: note = MPK136 VL (aa)
QVQLVQPPSVSGAPGQRVSISCTGTSSNIGTGYDVHWYQQFPGTSPKLLIYRNTNRPSGVPDRFSGSKSGSSASLAIAGLQAEDEADYYCQSYDRSVSGWVFGGGTKLTVL

SEQ ID NO: 425 - DNA, 361 bp, synthetic construct
  source 1..361: mol_type = other DNA; note = MPK141 VH (nt)
gaggtgcagctggtggagtctgggggaggcttggtcaagcctggagggtccctgaggctctcctgtgcagcctctggattcaccatcagtggctactatatgacctggatccgccaggctccagggaaggggctggagtggatttcatatattagtggtagtagtagttatacaaattacgcagactctgtgaagggccgattcaccatctccagagacaatggcaggaattcagtctatctccaaatgaacagcctgagagccgacgacacgggtgtttattactgtgcgagggaggagacgccgtcacctggcccctttgactcctggggccagggaatcctggtcaccgtctcctcag

SEQ ID NO: 426 - protein, 120 aa, synthetic construct
  source 1..120: note = MPK141 VH (aa)
EVQLVESGGGLVKPGGSLRLSCAASGFTISGYYMTWIRQAPGKGLEWISYISGSSSYTNYADSVKGRFTISRDNGRNSVYLQMNSLRADDTGVYYCAREETPSPGPFDSWGQGILVTVSS

SEQ ID NO: 427 - protein, 13 aa, synthetic construct
  source 1..13: note = MPK141 CDR-H3 (aa)
AREETPSPGPFDS

SEQ ID NO: 428 - DNA, 334 bp, synthetic construct
  source 1..334: mol_type = other DNA; note = MPK141 VL (nt)
cagtctgtgttgacgcagccgccctcagtgtctggggccccagggcagaggatcattatctcctgcattggcagcagctccaacatcgggacaggttatgatgtccactggtaccagcaacttccaggaagagcccccaaactcctcatctctcgtaacaccaatcggccctcagggatccctgaccgattctctggctccaagtctggcacctcagcctccctggccatcactgggctccaggctgacgatgaggctgattattactgccagtcctatgacaggggtctggatggttgggtgttcggcggagggaccaagctgaccgtcctag

SEQ ID NO: 429 - protein, 111 aa, synthetic construct
  source 1..111: note = MPK141 VL (aa)
QSVLTQPPSVSGAPGQRIIISCIGSSSNIGTGYDVHWYQQLPGRAPKLLISRNTNRPSGIPDRFSGSKSGTSASLAITGLQADDEADYYCQSYDRGLDGWVFGGGTKLTVL

SEQ ID NO: 430 - DNA, 358 bp, synthetic construct
  source 1..358: mol_type = other DNA; note = MPK142-v1 VH (nt) and MPK142-v2 VH (nt)
gaggtgcagctggtggagtctggcggaggcctggtcgagccgggggggtccctgagactctcctgtgcagcgtctggattcaccatcagtggctatggcatgagctgggtccggcaggctccagggaaggggctggagtgggtctcgtccattagtgctagtagtagttacatatattatggagacgcaatgaagggccgaatcaccatctccagagacaacggctggaactcaatgtatctgcaaatgaacagcctgagaaccgaggacacggctgtctattactgtgcgacagcgcctatagaaccgccatacttcgatctgtggggccgtggcaccctggtcactgtctcctcag

SEQ ID NO: 431 - protein, 119 aa, synthetic construct
  source 1..119: note = MPK142-v1; MPK142-v2; MPK145 VH (aa)
EVQLVESGGGLVEPGGSLRLSCAASGFTISGYGMSWVRQAPGKGLEWVSSISASSSYIYYGDAMKGRITISRDNGWNSMYLQMNSLRTEDTAVYYCATAPIEPPYFDLWGRGTLVTVSS

SEQ ID NO: 432 - protein, 12 aa, synthetic construct
  source 1..12: note = MPK142-v1; MPK 142-v2; MPK145 CDR-H3 (aa)
ATAPIEPPYFDL

SEQ ID NO: 433 - DNA, 334 bp, synthetic construct
  source 1..334: mol_type = other DNA; note = MPK142-v1 VL (nt)
cagtctgtgctgactcagccgccctcagtgtctggggccccagggcagagggtcaccatctcctgcactgggagtagctccaacatcggggcaggttatgatgtgcactggtatcaacagtttccaggcaaagcccccaaagtcctcatctatggtaacaacaatcggccctcaggggtccctgaccgattcgctgcctccaactctggcgcttcagcctccctggccatcactgggctccaggctgaggatgaggctgcttatttctgccagtcctatgacaggagactgagtggtccggttttcggcggagggaccaagctgaccgtcctag

SEQ ID NO: 434 - protein, 111 aa, synthetic construct
  source 1..111: note = MPK142-v1 (aa)
QSVLTQPPSVSGAPGQRVTISCTGSSSNIGAGYDVHWYQQFPGKAPKVLIYGNNNRPSGVPDRFAASNSGASASLAITGLQAEDEAAYFCQSYDRRLSGPVFGGGTKLTVL

SEQ ID NO: 435 - DNA, 322 bp, synthetic construct
  source 1..322: mol_type = other DNA; note = MPK142-v2 VL (nt)
gacatccagttgacccagactccaacctccctgtctgcgtctgtaggagacagagtcaccttcacttgccgggcaagtcagaacattggtttctatttaaattggtatcagcagaagccagggaaagcccctaggctgctgatctatggtgcatccaatttgcaaagtggggtcccatcaaggttcagtggcagtggggctgggacagatttctctctcaccatcagcagcctgcagcctgaagattttgcaacttactactgtcaacagagtttcaatacgccccggacattcggccaagggaccaaggtagaaatcaatc

SEQ ID NO: 436 - protein, 107 aa, synthetic construct
  source 1..107: note = MPK142-v2 VL (aa)
DIQLTQTPTSLSASVGDRVTFTCRASQNIGFYLNWYQQKPGKAPRLLIYGASNLQSGVPSRFSGSGAGTDFSLTISSLQPEDFATYYCQQSFNTPRTFGQGTKVEIN

SEQ ID NO: 437 - protein, 6 aa, synthetic construct
  source 1..6: note = MPK142-v2 CDR-L1 (aa)
QNIGFY

SEQ ID NO: 438 - protein, 9 aa, synthetic construct
  source 1..9: note = MPK142-v2 CDR-L3 (aa)
QQSFNTPRT

SEQ ID NO: 439 - DNA, 361 bp, synthetic construct
  source 1..361: mol_type = other DNA; note = MPK144 VH (nt)
gaggtgcagctggtggagtctgggggaggcttggtcaggcctggagggtccctgagactctcctgtgcagcctctggattcaccttcactggctactacatgatgtggatccgccaggctccagggaaggggctggagtggatttcagacattagtagtagtagtagttacacaaactacgctgactctgtgaagggccgattcgccatctccagagacaacgcccagaacacactgtatctgcaaatgaacagcctcagagccgaggacacggctgtgtattattgtgcgacagaacaggtgccgtcacctggcccctttgacctctggggccagggaaccctggtcaccgtctcctcag

SEQ ID NO: 440 - protein, 120 aa, synthetic construct
  source 1..120: note = MPK144 VH (aa)
EVQLVESGGGLVRPGGSLRLSCAASGFTFTGYYMMWIRQAPGKGLEWISDISSSSSYTNYADSVKGRFAISRDNAQNTLYLQMNSLRAEDTAVYYCATEQVPSPGPFDLWGQGTLVTVSS

SEQ ID NO: 441 - protein, 8 aa, synthetic construct
  source 1..8: note = MPK144 CDR-H1 (aa)
GFTFTGYY

SEQ ID NO: 442 - protein, 8 aa, synthetic construct
  source 1..8: note = MPK144 CDR-H2 (aa)
ISSSSSYT

SEQ ID NO: 443 - DNA, 334 bp, synthetic construct
  source 1..334: mol_type = other DNA; note = MPK144 VL (nt)
cagtctgtgttgacgcagccgccctcagtgtctggggccccagggcagagggtcaccatctcctgcactgggagcagctccaacttcggggcaggttatgatgtacactggtaccaacagtttccaggaacatcccccaaactcctcatctatcgtaataccaatcggccctcaggggtccctgaccgattctctggctccaagtctggcacctcagcctccctggccatcactgggctccaggctgaggatgaggctgattattactgccagtcctatgacagaagcctgactggatgggttttcggcggagggaccaagctgaccgtcctag

SEQ ID NO: 444 - protein, 111 aa, synthetic construct
  source 1..111: note = MPK144 VL (aa)
QSVLTQPPSVSGAPGQRVTISCTGSSSNFGAGYDVHWYQQFPGTSPKLLIYRNTNRPSGVPDRFSGSKSGTSASLAITGLQAEDEADYYCQSYDRSLTGWVFGGGTKLTVL

SEQ ID NO: 445 - protein, 9 aa, synthetic construct
  source 1..9: note = MPK144; MPK151 CDR-L1 (aa)
SSNFGAGYD

SEQ ID NO: 446 - protein, 11 aa, synthetic construct
  source 1..11: note = MPK144 CDR-L3 (aa)
QSYDRSLTGWV

SEQ ID NO: 447 - DNA, 358 bp, synthetic construct
  source 1..358: mol_type = other DNA; note = MPK145 VH (nt)
gaggtgcaactggtggagtctggcggaggcctggtcgagccgggggggtccctgagactctcctgtgcagcgtctggattcaccatcagtggctatggcatgagctgggtccggcaggctccagggaaggggctggagtgggtctcgtccattagtgctagtagtagttacatatattatggagacgcaatgaagggccgaatcaccatctccagagacaacggctggaactcaatgtatctgcaaatgaacagcctgagaaccgaggacacggctgtctattactgtgcgacagcgcctatagaaccgccatacttcgatctgtggggccgtggcaccctggtcactgtctcctcag

SEQ ID NO: 448 - DNA, 334 bp, synthetic construct
  source 1..334: mol_type = other DNA; note = MPK145 VL (nt)
cagtctgtcgtgacgcagccgccctcagtgtctggggccccagggcagagggtcaccatctcctgcactgggagtagctccaacatcggggcaggttatgatgtgcactggtatcaacagtttccaggcaaagcccccaaagtcctcatctatggtaacaacaatcggccctcaggggtccctgaccgattcgctgcctccaactctggcgcttcagcctccctggccatcactgggctccaggctgaggatgaggctgcttatttctgccagtcctatgacaggagactgagtggtccggttttcggcggagggaccaagctgaccgtcctag

SEQ ID NO: 449 - protein, 111 aa, synthetic construct
  source 1..111: note = MPK145 VL (aa)
QSVVTQPPSVSGAPGQRVTISCTGSSSNIGAGYDVHWYQQFPGKAPKVLIYGNNNRPSGVPDRFAASNSGASASLAITGLQAEDEAAYFCQSYDRRLSGPVFGGGTKLTVL

SEQ ID NO: 450 - DNA, 361 bp, synthetic construct
  source 1..361: mol_type = other DNA; note = MPK146 VH (nt)
caggtgcagctggtggagtctgggggaggcttggtcaagcctggagggtccctgagactctcctgtgcagcctctggattcaccttcagtggatactacatgatatggatccgccaggctccagggaaggggctggagtggattgcatccattagtggtagtagtaattacatagactacgctgactctgtgaggggccgattcaccatctccagagacaacgcccggaatattttggatctccaaatgaacagcctgagagccggggacacggctctgtattattgtgcgacagaacaggtgccgtcacctggcccctttgacctctggggccagggaaccctggtcaccgtctcctcag

SEQ ID NO: 451 - protein, 120 aa, synthetic construct
  source 1..120: note = MPK146 VH (aa)
QVQLVESGGGLVKPGGSLRLSCAASGFTFSGYYMIWIRQAPGKGLEWIASISGSSNYIDYADSVRGRFTISRDNARNILDLQMNSLRAGDTALYYCATEQVPSPGPFDLWGQGTLVTVSS

SEQ ID NO: 452 - protein, 8 aa, synthetic construct
  source 1..8: note = MPK146 CDR-H1 (aa)
GFTFSGYY

SEQ ID NO: 453 - protein, 8 aa, synthetic construct
  source 1..8: note = MPK146 CDR-H2 (aa)
ISGSSNYI

SEQ ID NO: 454 - DNA, 334 bp, synthetic construct
  source 1..334: mol_type = other DNA; note = MPK146 VL (nt)
cagtctgtgctgacgcagccgccctcagtgtctggggccccagggcagagggtcaccatctcctgcactgggagcaactccaatctcggggcaggttatgatgtacactggtaccaacagtttccaggaacatcccccaaactcctcatctatcgtaataccaatcggccctcaggggtccctgaccgattcgctggctccaagtctggcacctcagcctccctggccatcactgggctccaggctgaggatgaggctgattattactgccagtcctatgacagaagcctgagtggatgggttttcggcggagggaccaagctgaccgtcctag

SEQ ID NO: 455 - protein, 111 aa, synthetic construct
  source 1..111: note = MPK146 VL (aa)
QSVLTQPPSVSGAPGQRVTISCTGSNSNLGAGYDVHWYQQFPGTSPKLLIYRNTNRPSGVPDRFAGSKSGTSASLAITGLQAEDEADYYCQSYDRSLSGWVFGGGTKLTVL

SEQ ID NO: 456 - protein, 9 aa, synthetic construct
  source 1..9: note = MPK146 CDR-L1 (aa)
NSNLGAGYD

SEQ ID NO: 457 - DNA, 361 bp, synthetic construct
  source 1..361: mol_type = other DNA; note = MPK149 VH (nt)
caggtgcagctggtggagtctgggggaggcttggtcaagcctggagggtccctaagactctcctgtgaagcctctggattcaccataagtggctacttcatgatctggatccgccaggctccagggaaggggctggagtggatttcatccattagtggtagtagtacttacacagactacgctgagtctgtgaagggccgattcaccatctccagagacaacggccggagcacactgtatctccaaatgaacagcctgagagtcgaggacacggctgtgtattattgtgcgacggaacagacgccgtcacctggcccctttgacctctggggccagggaaccctggtcaccgtctcctcag

SEQ ID NO: 458 - protein, 120 aa, synthetic construct
  source 1..120: note = MPK149 VH (aa)
QVQLVESGGGLVKPGGSLRLSCEASGFTISGYFMIWIRQAPGKGLEWISSISGSSTYTDYAESVKGRFTISRDNGRSTLYLQMNSLRVEDTAVYYCATEQTPSPGPFDLWGQGTLVTVSS

SEQ ID NO: 459 - protein, 8 aa, synthetic construct
  source 1..8: note = MPK149 CDR-H1 (aa)
GFTISGYF

SEQ ID NO: 460 - DNA, 334 bp, synthetic construct
  source 1..334: mol_type = other DNA; note = MPK149 VL (nt)
cagtctgtcgtgacgcagccgccctcagtgtctggggccccagggcagagggtcaccatctcctgcactgggaccagctccaacatcggggcaggttatgatgtacactggtaccagcagtttccaggaacatcccccagactcctcatcaatcgtaataccaatcggccctcaggggtccctgaccgattctctggctcccagtctggctcctcagcctccctggccatcgctgggctccaggctgaggatgaggctgattattactgccagtcctatgacagaagtgtgagtggatgggttttcggcggtgggaccaagctgaccgtcctag

SEQ ID NO: 461 - protein, 111 aa, synthetic construct
  source 1..111: note = MPK149 VL (aa)
QSVVTQPPSVSGAPGQRVTISCTGTSSNIGAGYDVHWYQQFPGTSPRLLINRNTNRPSGVPDRFSGSQSGSSASLAIAGLQAEDEADYYCQSYDRSVSGWVFGGGTKLTVL

SEQ ID NO: 462 - DNA, 361 bp, synthetic construct
  source 1..361: mol_type = other DNA; note = MPK150-v2 VH (nt)
gaggtgcagctggtggagtctgggggaggcttggtcaagcctggagggtccctaagactctcctgtgaagcctctggaatctccctcagtggatactacatgatctggatccgccaggctcccgggaagggactggagtggatttcatccatcagtggtagtagtacttatattgactacgctgactctgtgaggggccgattcaccatctccagacacaacggccagaacacaatttatctccagatgaacagcctgagagtcgaggacacggctgtttatttttgtgcgacagaacaaacgccgtcccctggcccctttgaattgtggggccggggaatcctggtcaccgtctcatcag

SEQ ID NO: 463 - protein, 120 aa, synthetic construct
  source 1..120: note = MPK150-v2 VH (aa)
EVQLVESGGGLVKPGGSLRLSCEASGISLSGYYMIWIRQAPGKGLEWISSISGSSTYIDYADSVRGRFTISRHNGQNTIYLQMNSLRVEDTAVYFCATEQTPSPGPFELWGRGILVTVSS

SEQ ID NO: 464 - protein, 8 aa, synthetic construct
  source 1..8: note = MPK150-v2 CDR-H2 (aa)
ISGSSTYI

SEQ ID NO: 465 - DNA, 334 bp, synthetic construct
  source 1..334: mol_type = other DNA; note = MPK150-v2 VL (nt)
cagtctgtgttgacgcagccgccctcagtgtctggggccccagggcagagggtcaccatcttctgcactgggaccagctccaacatcgggacaggttatgatgtacactggtaccaacagtttccaggaacatcccccaaactcctcatctttcgtaattccaatcggccctcaggggtccctgaccgattctctggctccaagtctggctcctcagcctccctggccatcgctgggctccaggctgaggatgaggctgattattactgccagtcgtatgacagaggtgtgaatggatgggttttcggcggagggaccaagctgaccgtcctag

SEQ ID NO: 466 - protein, 111 aa, synthetic construct
  source 1..111: note = MPK150-v2 VL (aa)
QSVLTQPPSVSGAPGQRVTIFCTGTSSNIGTGYDVHWYQQFPGTSPKLLIFRNSNRPSGVPDRFSGSKSGSSASLAIAGLQAEDEADYYCQSYDRGVNGWVFGGGTKLTVL

SEQ ID NO: 467 - skipped

SEQ ID NO: 468 - protein, 11 aa, synthetic construct
  source 1..11: note = MPK150-v2 CDR-L3 (aa)
QSYDRGVNGWV

SEQ ID NO: 469 - DNA, 361 bp, synthetic construct
  source 1..361: mol_type = other DNA; note = MPK151 VH (nt)
gaggtgcagctggtggagtcggggggaggcttggtcaagcctggagggtccctgagactctcctgtgcagcctctggattgaccatcagtggctactacatgatctggatccgccaggctccaggaaaggggctggagtggatttcagacattagtgcgagcagtacttacacagactacgctgagtctgtgaagggccgattcagcatctccagagacaacggccagaacacagtgtatctccaaatgaacagcctgagagccgaagacacggctgtttattattgtgcgacagaacaaacgccgtcacctggcccctttgacctctggggccagggaaccctggtcaccgtctcttcag

SEQ ID NO: 470 - protein, 120 aa, synthetic construct
  source 1..120: note = MPK151 VH (aa)
EVQLVESGGGLVKPGGSLRLSCAASGLTISGYYMIWIRQAPGKGLEWISDISASSTYTDYAESVKGRFSISRDNGQNTVYLQMNSLRAEDTAVYYCATEQTPSPGPFDLWGQGTLVTVSS

SEQ ID NO: 471 - protein, 8 aa, synthetic construct
  source 1..8: note = MPK151; MPK152 CDR-H1 (aa)
GLTISGYY

SEQ ID NO: 472 - DNA, 334 bp, synthetic construct
  source 1..334: mol_type = other DNA; note = MPK151 VL (nt)
cagtctgtgctgacgcagccgccctcagtgtctggggccccagggcagaagatcaccatctcctgcactgggggcagctccaacttcggggcaggttatgatgtacactggtaccaacagtttccaggaacatcccccaaactcctcatctatcgtaatacctttcggccctcaggggtccctgaccgattctctggctccaagtctggaacctcagcctccctggccatcactgggctccaggctgaggatgaggctgactattactgtcagtcctatgacagaagcctgagtggatgggttttcggcggcgggaccaagctgaccgtcctag

SEQ ID NO: 473 - protein, 111 aa, synthetic construct
  source 1..111: note = MPK151 VL (aa)
QSVLTQPPSVSGAPGQKITISCTGGSSNFGAGYDVHWYQQFPGTSPKLLIYRNTFRPSGVPDRFSGSKSGTSASLAITGLQAEDEADYYCQSYDRSLSGWVFGGGTKLTVL

SEQ ID NO: 474 - DNA, 361 bp, synthetic construct
  source 1..361: mol_type = other DNA; note = MPK152 VH (nt)
gaggtgcagctggtggagtctgggggaggcttggtcaagcctggcggatccctcagactctcctgtgcagcctctggattaaccatcagtggctactacatgatctggattcggcaggctccagggaagggactggagtggatttcatacataagtgggagcagttcctatacaaattacgctgactctgtgaagggccgattcaccatctccagagacaacggccagaacacactgtttctccaaatggacagcctgagagtcgaggatacggctgtttatttttgtgcgacagaagagactccgtcacctggccccttcggcctctggggccagggaaccctggtcaccgtctcctcag

SEQ ID NO: 475 - protein, 120 aa, synthetic construct
  source 1..120: note = MPK152 VH (aa)
EVQLVESGGGLVKPGGSLRLSCAASGLTISGYYMIWIRQAPGKGLEWISYISGSSSYTNYADSVKGRFTISRDNGQNTLFLQMDSLRVEDTAVYFCATEETPSPGPFGLWGQGTLVTVSS

SEQ ID NO: 476 - protein, 13 aa, synthetic construct
  source 1..13: note = MPK152 CDR-H3 (aa)
ATEETPSPGPFGL

SEQ ID NO: 477 - DNA, 334 bp, synthetic construct
  source 1..334: mol_type = other DNA; note = MPK152 VL (nt)
cagtctgtcgtgacgcagccgccctcagtgtctggggccccagggcagagggtcaccatctcctgcactgggaccagctccaacatcggggcaggttatgatgtacactggtatcaacaatttcccggaacatcccccaaactcctcatctttcgtaataccaatcggccctcaggggtccctgaccgattctctggctccacgtctggctcctcagcctccctggccatcactgggctccagactgaggatgaggctgattattactgccaatcttatgacagaagcctggatggctgggttttcggcggagggaccgagctgaccgtcctag

SEQ ID NO: 478 - protein, 111 aa, synthetic construct
  source 1..111: note = MPK152 VL (aa)
QSVVTQPPSVSGAPGQRVTISCTGTSSNIGAGYDVHWYQQFPGTSPKLLIFRNTNRPSGVPDRFSGSTSGSSASLAITGLQTEDEADYYCQSYDRSLDGWVFGGGTELTVL

SEQ ID NO: 479 - DNA, 361 bp, synthetic construct
  source 1..361: mol_type = other DNA; note = MPK153 VH (nt)
gaggtgcatctggtggagtctgggggaggcttggtcaagcccggagggtctctaagactctcctgtgcagcctctggattcaccttcactggctacttcatgatctggatccgccaggctccagggaaggggctggagtggatttcatacatcagtagcagtagtaattacattaattatgctgactctgtgaagggccgattcaccatctccagggacaacggccagaacacactgtatctccaaatgaacagcctgagacccgacgacacggctgtgtattattgtgcgacagagcaggtgccgtcgcctggccccttcgacctctggggccagggaaccctggtcaccgtctcctcgg

SEQ ID NO: 480 - protein, 120 aa, synthetic construct
  source 1..120: note = MPK153 VH (aa)
EVHLVESGGGLVKPGGSLRLSCAASGFTFTGYFMIWIRQAPGKGLEWISYISSSSNYINYADSVKGRFTISRDNGQNTLYLQMNSLRPDDTAVYYCATEQVPSPGPFDLWGQGTLVTVSS

SEQ ID NO: 481 - protein, 8 aa, synthetic construct
  source 1..8: note = MPK153 CDR-H1 (aa)
GFTFTGYF

SEQ ID NO: 482 - DNA, 334 bp, synthetic construct
  source 1..334: mol_type = other DNA; note = MPK153 VL (nt)
cagtctgtgctgacgcagccgccctcagtgtctggggccccagggcagagggtcatcatctcctgcactgggaccagctccaacatcggggcaggttatgatgtacactggtaccaacaatttccaggagcatcccccaaactcctcatctttcgtaatacagatcggccctcaggggtccctgaccgattctctggctccaagtctggctcctcagcctccctggccatcgctgggctccaggctgaggatgaggctgattattactgccagtcctatgacagaagcctggatggatgggttttcggcggagggaccaaggtgaccgtcctag

SEQ ID NO: 483 - protein, 111 aa, synthetic construct
  source 1..111: note = MPK153 VL (aa)
QSVLTQPPSVSGAPGQRVIISCTGTSSNIGAGYDVHWYQQFPGASPKLLIFRNTDRPSGVPDRFSGSKSGSSASLAIAGLQAEDEADYYCQSYDRSLDGWVFGGGTKVTVL

SEQ ID NO: 484 - DNA, 361 bp, synthetic construct
  source 1..361: mol_type = other DNA; note = MPK155 VH (nt)
gaggtgcagctggtggagtctgggggaggcttggtcaagcctggagggtccctgagactctcctgtgaagcctctggaatctccctcagtgggtactacatgatctggatccgccaggctccagggaagggactggagtggatttcatccataagtagtagtagtaattatattgactacgctgactctgtgaggggccgattcaccatctccagacacaacggccagaacacactctatctgcagatgaacagtctgagagtcgaggacacggctgtttatttttgtgcgacagaacaaacgccgtcccctggcccctttgacttgtggggccagggaatcctggtcaccgtctcatcag

SEQ ID NO: 485 - protein, 120 aa, synthetic construct
  source 1..120: note = MPK155 VH (aa)
EVQLVESGGGLVKPGGSLRLSCEASGISLSGYYMIWIRQAPGKGLEWISSISSSSNYIDYADSVRGRFTISRHNGQNTLYLQMNSLRVEDTAVYFCATEQTPSPGPFDLWGQGILVTVSS

SEQ ID NO: 486 - DNA, 334 bp, synthetic construct
  source 1..334: mol_type = other DNA; note = MPK155 VL (nt)
cagtctgtgctgacgcagccgccctccgtctctggggccccagggcagagggtcaccatcttctgcactgggaccagctccaacctcggggcaggttatgatgtccactggtatcaacagtttccaggaacatcgcccaaactcctcatctatcgttataccaatcggccctcaggggtccctgaccgattctctggctccaagtctggctcctcagcctccctggccatcgctgggctccaggctgaggatgaggctgattattactgccagtcctatgacagaagtgtcagtggatgggttttcggcggagggaccaagctgaccgtcctag

SEQ ID NO: 487 - protein, 111 aa, synthetic construct
  source 1..111: note = MPK155 VL (aa)
QSVLTQPPSVSGAPGQRVTIFCTGTSSNLGAGYDVHWYQQFPGTSPKLLIYRYTNRPSGVPDRFSGSKSGSSASLAIAGLQAEDEADYYCQSYDRSVSGWVFGGGTKLTVL

SEQ ID NO: 488 - protein, 9 aa, synthetic construct
  source 1..9: note = MPK155 CDR-L1 (aa)
SSNLGAGYD

SEQ ID NO: 489 - skipped

SEQ ID NO: 490 - DNA, 358 bp, synthetic construct
  source 1..358: mol_type = other DNA; note = MPK157 VH (nt)
gaggtgcagctggtggagtctgggggaggcttggtcaagcctggagggtccctgagactctcctgttcagcgtctggattcaccctcagtggctacaacatgaactgggtccgccaggctccagggaaggggctggagtggatttcatatattagtgccagtagtagttacataaactacgcggactctgtgaagggccgattcaccatctccagagacaacggcaggagttcattgtatctgcaaatgaacagcctgcgagccgaggacaccgctgtgtatttctgtacgacgcaggcggagttgccgggatggttcgacccctggggccagggaaccctggtcaccgtctcttcag

SEQ ID NO: 491 - protein, 119 aa, synthetic construct
  source 1..119: note = MPK157 VH (aa)
EVQLVESGGGLVKPGGSLRLSCSASGFTLSGYNMNWVRQAPGKGLEWISYISASSSYINYADSVKGRFTISRDNGRSSLYLQMNSLRAEDTAVYFCTTQAELPGWFDPWGQGTLVTVSS

SEQ ID NO: 492 - protein, 8 aa, synthetic construct
  source 1..8: note = MPK157 CDR-H1 (aa)
GFTLSGYN

SEQ ID NO: 493 - protein, 12 aa, synthetic construct
  source 1..12: note = MPK157 CDR-H3 (aa)
TTQAELPGWFDP

SEQ ID NO: 494 - DNA, 334 bp, synthetic construct
  source 1..334: mol_type = other DNA; note = MPK157 VL (nt)
cagtctgtgttgacgcagccgccctcaatgtctggggccccagggcagagggtcaccatctcctgcactgggagcagttccaacatcggggcaggttatgatgtaaactggtatcggcagcttccaggaacagccccccaactcctcatctatcgcaataccaatcggccctcaggggtccctgaccgattctctggctccaactctggcacctcagcctccctggccatcaccgggctccaggctgacgatgaggctcattattactgccagtcctatgaccgcagcctgagtggttgggtattcggcggagggaccaagctgaccgtcctag

SEQ ID NO: 495 - protein, 111 aa, synthetic construct
  source 1..111: note = MPK157 VL (aa)
QSVLTQPPSMSGAPGQRVTISCTGSSSNIGAGYDVNWYRQLPGTAPQLLIYRNTNRPSGVPDRFSGSNSGTSASLAITGLQADDEAHYYCQSYDRSLSGWVFGGGTKLTVL

SEQ ID NO: 496 - DNA, 361 bp, synthetic construct
  source 1..361: mol_type = other DNA; note = MPK158 VH (nt)
gaggtgcagctggtggaatctgggggaggcctggtcaaacctggagggtccctgagactgtcctgcgcagcctctggattctccatctctggattctacatgatctggatccgtcaggctccagggaaggggctggaatggatttcagacattagtgccagttcttcttacatagactacgccgactctgtgaagggccgattcaccatctccagagacaacggccagaacacagtccatctactgatgaacaacctgagagccgacgacacgggagtgtattattgtgcgagagagcagacgccgtcacctggcccctttgacctctggggccagggaaccctggtcaccgtctccacag

SEQ ID NO: 497 - protein, 120 aa, synthetic construct
  source 1..120: note = MPK158 VH (aa)
EVQLVESGGGLVKPGGSLRLSCAASGFSISGFYMIWIRQAPGKGLEWISDISASSSYIDYADSVKGRFTISRDNGQNTVHLLMNNLRADDTGVYYCAREQTPSPGPFDLWGQGTLVTVST

SEQ ID NO: 498 - protein, 8 aa, synthetic construct
  source 1..8: note = MPK158 CDR-H1 (aa)
GFSISGFY

SEQ ID NO: 499 - protein, 13 aa, synthetic construct
  source 1..13: note = MPK158 CDR-H3 (aa)
AREQTPSPGPFDL

SEQ ID NO: 500 - DNA, 334 bp, synthetic construct
  source 1..334: mol_type = other DNA; note = MPK158 VL (nt)
cagcctgtgctgactcagccgccctcagtgtctggggccccagggcagagggtcaccatctcctgcactgggcgcagctccaacatcggggcaggttatgatgttcactggtaccaacaccttcctggaacatcacccaaactcctcatctatcgtaattccaatcggccctcaggggtccctgaccgattctctggctccacgtctggcacctcagcctccctggccatcactgggctccaggctgacgatgaggctgattattactgccaggcctatgacagaatcctgggtggatgggctttcggcggggggaccaagctgaccgtcctag

SEQ ID NO: 501 - protein, 111 aa, synthetic construct
  source 1..111: note = MPK158 VL (aa)
QPVLTQPPSVSGAPGQRVTISCTGRSSNIGAGYDVHWYQHLPGTSPKLLIYRNSNRPSGVPDRFSGSTSGTSASLAITGLQADDEADYYCQAYDRILGGWAFGGGTKLTVL

SEQ ID NO: 502 - protein, 11 aa, synthetic construct
  source 1..11: note = MPK158 CDR-L3 (aa)
QAYDRILGGWA

SEQ ID NO: 503 - DNA, 361 bp, synthetic construct
  source 1..361: mol_type = other DNA; note = MPK162 VH (nt)
gaggtgcagctggtggagtctgggggaggcttggtcaagcctggagattccctgagactctcctgcgcagcctctgggttcagcctcagttcctactacatgttctggatccgccaggctccagggaagggactggagtgggtttcagacatcagtggaggtagtactttcacaaactacgccgactctgtgaagggccgattcaccatctccagagacaacgccaagaaagtactgtatctgcaaatgaacagcctgagagtcgacgactcggctgtttattattgtgtgagagggtcttcgatcagcccaaactggttcgagtcctggggccagggagccctggtcaccgtctcctcag

SEQ ID NO: 504 - protein, 120 aa, synthetic construct
  source 1..120: note = MPK162 VH (aa)
EVQLVESGGGLVKPGDSLRLSCAASGFSLSSYYMFWIRQAPGKGLEWVSDISGGSTFTNYADSVKGRFTISRDNAKKVLYLQMNSLRVDDSAVYYCVRGSSISPNWFESWGQGALVTVSS

SEQ ID NO: 505 - protein, 8 aa, synthetic construct
  source 1..8: note = MPK162 CDR-H1 (aa)
GFSLSSYY

SEQ ID NO: 506 - protein, 8 aa, synthetic construct
  source 1..8: note = MPK162 CDR-H2 (aa)
ISGGSTFT

SEQ ID NO: 507 - protein, 13 aa, synthetic construct
  source 1..13: note = MPK162 CDR-H3 (aa)
VRGSSISPNWFES

SEQ ID NO: 508 - DNA, 337 bp, synthetic construct
  source 1..337: mol_type = other DNA; note = MPK162 VL (nt)
cagtctgtgctgacgcagccgccctcggtgtctggggccccagggcagagggtcaccatctcctgcactgggaccagctccaacatcggggcaggttatgatgttcactggtaccagcaggttccgggaacagcccccaaactcctcatctatggtaacagcaatcggccctcaggggtccctgaccgattctctggctccaagtctggcaccgcagcctccctggccatcaatgggctccagactgaggatgaggctgagtattactgccagtcttatgaccgcagcctgactggttcttatgtcttcggaagtgggaccagggtcttcgtccgag

SEQ ID NO: 509 - protein, 112 aa, synthetic construct
  source 1..112: note = MPK162 VL (aa)
QSVLTQPPSVSGAPGQRVTISCTGTSSNIGAGYDVHWYQQVPGTAPKLLIYGNSNRPSGVPDRFSGSKSGTAASLAINGLQTEDEAEYYCQSYDRSLTGSYVFGSGTRVFVR

SEQ ID NO: 510 - skipped

SEQ ID NO: 511 - protein, 12 aa, synthetic construct
  source 1..12: note = MPK162 CDR-L3 (aa)
QSYDRSLTGSYV

SEQ ID NO: 512 - DNA, 372 bp, synthetic construct
  source 1..372: mol_type = other DNA; note = MPK174-v2 VH (nt)
gaggtgcagctggtggagtctgggggaggcttggtcaagcctggagggtccctgagactctcctgtgcagcctctcgatttaagttcggtgactactacatgagctggatccgccaggctccagggaaggggctggagtgggtttcaaacattagtcctagtagtagttatacaaactacgcagactctgtgaagggccgattcaccatctccagagacaacgccaagaacttactgtatctgcaaatgaacagcctgagagccgaggacacggctgtttattactgtgcgagagagtcctgtagtggtggtagctgctctctgcacggtatggacgtctggggccaagggaccacggtcaccgtcgcgtca

SEQ ID NO: 513 - protein, 124 aa, synthetic construct
  source 1..124: note = MPK174-v2 VH (aa)
EVQLVESGGGLVKPGGSLRLSCAASRFKFGDYYMSWIRQAPGKGLEWVSNISPSSSYTNYADSVKGRFTISRDNAKNLLYLQMNSLRAEDTAVYYCARESCSGGSCSLHGMDVWGQGTTVTVAS

SEQ ID NO: 514 - protein, 8 aa, synthetic construct
  source 1..8: note = MPK174-v2; MPK196 CDR-H1 (aa)
RFKFGDYY

SEQ ID NO: 515 - protein, 8 aa, synthetic construct
  source 1..8: note = MPK174-v2; MPK196 CDR-H2 (aa)
ISPSSSYT

SEQ ID NO: 516 - protein, 17 aa, synthetic construct
  source 1..17: note = MPK174-v2; MPK196 CDR-H3 (aa)
ARESCSGGSCSLHGMDV

SEQ ID NO: 517 - DNA, 334 bp, synthetic construct
  source 1..334: mol_type = other DNA; note = MPK174-v2 VL (nt)
cagtctgtgctgacgcagccgccctcagtgtctggggccccagggcagagggtcaccatctcctgcaccgggagcagctccaacatcggggcaggttatgatgtacactggtaccagcagcttccaggaacagcccccaaactcctcatctatgataacagcaatcggccctcaggggtccctgaccgattctctggctccaagtctggcacctcagcctccctggccatcactggcctccgggctgaggatgacgctgattattactgccagtcctatgacaggagcctgggtggttcaatattcggcggagggaccaagctgaccgtcctag

SEQ ID NO: 518 - protein, 111 aa, synthetic construct
  source 1..111: note = MPK174-v2 VL (aa) and MPK196 VL (aa)
QSVLTQPPSVSGAPGQRVTISCTGSSSNIGAGYDVHWYQQLPGTAPKLLIYDNSNRPSGVPDRFSGSKSGTSASLAITGLRAEDDADYYCQSYDRSLGGSIFGGGTKLTVL

SEQ ID NO: 519 - skipped

SEQ ID NO: 520 - protein, 11 aa, synthetic construct
  source 1..11: note = MPK174-v2 CDR-L3 (aa) and MPK 196 CRD-L3 (aa)
QSYDRSLGGSI

SEQ ID NO: 521 - DNA, 334 bp, synthetic construct
  source 1..334: mol_type = other DNA; note = MPK190 VL (nt)
cagtctgtgctgacgcagccgccctcagtgtctggggcccccgggcagagggtcaccatctcctgcacagggagtaactccaacatcggggcaggatatgatgttcactggtaccaacatcttccaggaaaagcccccaaagtcctcatctatggtaacaacaatcggccctcagtggtccctgaccgattctctggctccaactctggcgcctcagcctccctggccatcagtgggctccaggctgaggatgaggctgattatttctgccagtcttatgacaggagactgagtggtccgatcttcggcggagggaccaagctgaccgtcctag

SEQ ID NO: 522 - protein, 111 aa, synthetic construct
  source 1..111: note = MPK190 VL (aa) and MPK 190-v1.1 VL (aa)
QSVLTQPPSVSGAPGQRVTISCTGSNSNIGAGYDVHWYQHLPGKAPKVLIYGNNNRPSVVPDRFSGSNSGASASLAISGLQAEDEADYFCQSYDRRLSGPIFGGGTKLTVL

SEQ ID NO: 523 - DNA, 372 bp, synthetic construct
  source 1..372: mol_type = other DNA; note = MPK196 VH (nt)
caggtgcagctggtggagtctgggggaggcttggtcaagcctggagggtccctgagactctcctgtgcagcctctcgatttaagttcggtgactactacatgagctggatccgccaggctccagggaaggggctggagtgggtttcaaacattagtcctagtagtagttatacaaactacgcagactctgtgaagggccgattcaccatctccagagacaacgccaacaacttactgtatctgcaaatgaacagcctgagagccgaggacacggctgtttattactgtgcgagagagtcgtgtagtggtggtagctgctctctgcacggtatggacgtctggggccaagggaccacggtcaccgtcgcgtca

SEQ ID NO: 524 - protein, 124 aa, synthetic construct
  source 1..124: note = MPK196 VH (aa)
QVQLVESGGGLVKPGGSLRLSCAASRFKFGDYYMSWIRQAPGKGLEWVSNISPSSSYTNYADSVKGRFTISRDNANNLLYLQMNSLRAEDTAVYYCARESCSGGSCSLHGMDVWGQGTTVTVAS

SEQ ID NO: 525 - DNA, 334 bp, synthetic construct
  source 1..334: mol_type = other DNA; note = MPK196 VL (nt)
cagtctgtgctgacgcagccgccctcagtgtctggggccccagggcagagggtcaccatctcctgcaccgggagcagctccaacatcggggcaggttatgatgtacactggtaccagcagcttccaggaacagcccccaaactcctcatctatgataacagcaatcggccctcaggggtccctgaccgattctctggctccaagtctggcacctcagcctccctggccatcactggcctccgggctgaggatgacgctgattattactgccagtcgtatgacaggagcctgggtggttcaatattcggcggagggaccaagctgaccgtcctag

SEQ ID NO: 526 - DNA, 361 bp, synthetic construct
  source 1..361: mol_type = other DNA; note = MPK204 VH (nt)
caggtacagctggtggagtctgggggaggcttggtcaagcctggagggtccctaagactctcctgtgaagcctctggactctccctcagtggctactacatgatctggatccgccaggctccagggaagggactggagtggatttcatccattagtagtagtagtaattacatagactacgctgactctgtgaagggccgattcaccatctccagacacaacggccagaacacactctatctccagatgaacagcctgagagtcgaggacacggctgtgtattattgtgcgacagaacaaacgccgtcccctggcccctttgacctctggggccagggaaccctggtcaccgtctcctcag

SEQ ID NO: 527 - protein, 120 aa, synthetic construct
  source 1..120: note = MPK204 VH (aa)
QVQLVESGGGLVKPGGSLRLSCEASGLSLSGYYMIWIRQAPGKGLEWISSISSSSNYIDYADSVKGRFTISRHNGQNTLYLQMNSLRVEDTAVYYCATEQTPSPGPFDLWGQGTLVTVSS

SEQ ID NO: 528 - protein, 8 aa, synthetic construct
  source 1..8: note = MPK204 CDR-H1 (aa)
GLSLSGYY

SEQ ID NO: 529 - DNA, 334 bp, synthetic construct
  source 1..334: mol_type = other DNA; note = MPK204 VL (nt)
cagtctgtgctgacgcagccgccctcagtgtctggggccccagggcagagggtcaccatctcctgcactgggaccagctccaacatcgggacaggttatgatgtacactggtaccaacagtttccaggaacatcccccaaactcctcatctatcgtaataccaatcggccctcaggggtccctgaccgattctctggctccaagtctggctcctcagcctccctggccatcgctgggctccaggctgaggatgaggctgattattactgccagtcgtatgacagaagtgtgagtggatgggttttcggcggagggaccaagctgaccgtcctag

SEQ ID NO: 530 - protein, 111 aa, synthetic construct
  source 1..111: note = MPK204 VL (aa)
QSVLTQPPSVSGAPGQRVTISCTGTSSNIGTGYDVHWYQQFPGTSPKLLIYRNTNRPSGVPDRFSGSKSGSSASLAIAGLQAEDEADYYCQSYDRSVSGWVFGGGTKLTVL

SEQ ID NO: 531 - DNA, 401 bp, synthetic construct
  source 1..401: mol_type = other DNA; note = MPH12 VH (co-nt)
accggtgtacattctcaggtgcagctggtggagagcggaggaggactggtgaagccaggaggatccctgaggctgagctgcgtggcctctggcttcaccttttctggcttctacatgaagtggatcagacaggctcctggcaagggcctggagtgggtgtccagcatctccgccagcggctcttacaccaactatgctgactccgtggagggcaggttcacagtgtctcgggataacgccaagaattccctgtttctgctgatgtcttccctgagagccgaggacacagccgtgtactattgtgctaggggcgattcctattgcggagcttccgcctgtcacggagagttctttcataagtggggccagggcaccctggtgacagtgagctctgcgtcgac

SEQ ID NO: 532 - protein, 126 aa, synthetic construct
  source 1..126: note = MPH12 VH (aa)
QVQLVESGGGLVKPGGSLRLSCVASGFTFSGFYMKWIRQAPGKGLEWVSSISASGSYTNYADSVEGRFTVSRDNAKNSLFLLMSSLRAEDTAVYYCARGDSYCGASACHGEFFHKWGQGTLVTVSS

SEQ ID NO: 533 - protein, 10 aa, synthetic construct
  source 1..10: note = MPH12; MPH12 VH-2CP (C111P, C112.2P); MPH12 VH-FRGL-S63A; MPH12 VH-FRGL-D62E; MPH12 VH-S63A; MPH12 VH-D62E; MPH12 VH-FRGL; MPH12 VH CDR-H1 (aa)
GFTFSGFYMK

SEQ ID NO: 534 - protein, 17 aa, synthetic construct
  source 1..17: note = MPH12; MPH12 VH-2CP (C111P, C112.2P); MPH12 VH-FRGL; MPH12 VH CDR-H2 (aa)
SISASGSYTNYADSVEG

SEQ ID NO: 535 - protein, 19 aa, synthetic construct
  source 1..19: note = MPH12; MPH12 VH-FRGL-S63A; MPH12 VH-FRGL-D62E; MPH12 VH-S63A; MPH12 VH-D62E; MPH12 VH-FRGL; MPH12 VH CDR-H3 (aa)
ARGDSYCGASACHGEFFHK

SEQ ID NO: 536 - DNA, 401 bp, synthetic construct
  source 1..401: mol_type = other DNA; note = MPH12 VH-FRGL VH (co-nt)
accggtgtacattctcaggtgcagctggtggagagcggaggaggactggtgaagccaggaggatccctgaggctgagctgcgcggcctctggcttcaccttttctggcttctacatgaagtggatcagacaggctcctggcaagggcctggagtgggtgtccagcatctccgccagcggctcttacaccaactatgctgactccgtggagggcaggttcacaatttctcgggataacgccaagaattccctgtatctgcagatgaactccctgagagccgaggacacagccgtgtactattgtgctaggggcgattcctattgcggagcttccgcctgtcacggagagttctttcataagtggggccagggcaccctggtgacagtgagctctgcgtcgac

SEQ ID NO: 537 - protein, 126 aa, synthetic construct
  source 1..126: note = MPH12 VH-FRGL VH (aa)
QVQLVESGGGLVKPGGSLRLSCAASGFTFSGFYMKWIRQAPGKGLEWVSSISASGSYTNYADSVEGRFTISRDNAKNSLYLQMNSLRAEDTAVYYCARGDSYCGASACHGEFFHKWGQGTLVTVSS

SEQ ID NO: 538 - DNA, 401 bp, synthetic construct
  source 1..401: mol_type = other DNA; note = MPH12 VH-D62E VH (co-nt)
accggtgtacattctcaggtgcagctggtggagagcggaggaggactggtgaagccaggaggatccctgaggctgagctgcgtggcctctggcttcaccttttctggcttctacatgaagtggatcagacaggctcctggcaagggcctggagtgggtgtccagcatctccgccagcggctcttacaccaactatgctgaatccgtggagggcaggttcacagtgtctcgggataacgccaagaattccctgtttctgctgatgtcttccctgagagccgaggacacagccgtgtactattgtgctaggggcgattcctattgcggagcttccgcctgtcacggagagttctttcataagtggggccagggcaccctggtgacagtgagctctgcgtcgac

SEQ ID NO: 539 - protein, 126 aa, synthetic construct
  source 1..126: note = MPH12 VH-D62E VH (aa)
QVQLVESGGGLVKPGGSLRLSCVASGFTFSGFYMKWIRQAPGKGLEWVSSISASGSYTNYAESVEGRFTVSRDNAKNSLFLLMSSLRAEDTAVYYCARGDSYCGASACHGEFFHKWGQGTLVTVSS

SEQ ID NO: 540 - protein, 17 aa, synthetic construct
  source 1..17: note = MPH12 VH-D62E; MPH12 VH-FRGL-D62E CDR-H2 (aa)
SISASGSYTNYAESVEG

SEQ ID NO: 541 - DNA, 401 bp, synthetic construct
  source 1..401: mol_type = other DNA; note = MPH12 VH-S63A VH (co-nt)
accggtgtacattctcaggtgcagctggtggagagcggaggaggactggtgaagccaggaggatccctgaggctgagctgcgtggcctctggcttcaccttttctggcttctacatgaagtggatcagacaggctcctggcaagggcctggagtgggtgtccagcatctccgccagcggctcttacaccaactatgctgacgcggtggagggcaggttcacagtgtctcgggataacgccaagaattccctgtttctgctgatgtcttccctgagagccgaggacacagccgtgtactattgtgctaggggcgattcctattgcggagcttccgcctgtcacggagagttctttcataagtggggccagggcaccctggtgacagtgagctctgcgtcgac

SEQ ID NO: 542 - protein, 126 aa, synthetic construct
  source 1..126: note = MPH12 VH-S63A VH (aa)
QVQLVESGGGLVKPGGSLRLSCVASGFTFSGFYMKWIRQAPGKGLEWVSSISASGSYTNYADAVEGRFTVSRDNAKNSLFLLMSSLRAEDTAVYYCARGDSYCGASACHGEFFHKWGQGTLVTVSS

SEQ ID NO: 543 - protein, 18 aa, synthetic construct
  source 1..18: note = MPH12 VH-S63A CDR-H2 (aa)
SSISASGSYTNYADAVEG

SEQ ID NO: 544 - DNA, 401 bp, synthetic construct
  source 1..401: mol_type = other DNA; note = MPH12 VH-FRGL-D62E VH (co-nt)
accggtgtacattctcaggtgcagctggtggagagcggaggaggactggtgaagccaggaggatccctgaggctgagctgcgcggcctctggcttcaccttttctggcttctacatgaagtggatcagacaggctcctggcaagggcctggagtgggtgtccagcatctccgccagcggctcttacaccaactatgctgaatccgtggagggcaggttcacaatttctcgggataacgccaagaattccctgtatctgcagatgaactccctgagagccgaggacacagccgtgtactattgtgctaggggcgattcctattgcggagcttccgcctgtcacggagagttctttcataagtggggccagggcaccctggtgacagtgagctctgcgtcgac

SEQ ID NO: 545 - protein, 126 aa, synthetic construct
  source 1..126: note = MPH12 VH-FRGL-D62E VH (aa)
QVQLVESGGGLVKPGGSLRLSCAASGFTFSGFYMKWIRQAPGKGLEWVSSISASGSYTNYAESVEGRFTISRDNAKNSLYLQMNSLRAEDTAVYYCARGDSYCGASACHGEFFHKWGQGTLVTVSS

SEQ ID NO: 546 - DNA, 401 bp, synthetic construct
  source 1..401: mol_type = other DNA; note = MPH12 VH-FRGL-S63A VH (co-nt)
accggtgtacattctcaggtgcagctggtggagagcggaggaggactggtgaagccaggaggatccctgaggctgagctgcgcggcctctggcttcaccttttctggcttctacatgaagtggatcagacaggctcctggcaagggcctggagtgggtgtccagcatctccgccagcggctcttacaccaactatgctgacgcggtggagggcaggttcacaatttctcgggataacgccaagaattccctgtatctgcagatgaactccctgagagccgaggacacagccgtgtactattgtgctaggggcgattcctattgcggagcttccgcctgtcacggagagttctttcataagtggggccagggcaccctggtgacagtgagctctgcgtcgac

SEQ ID NO: 547 - protein, 126 aa, synthetic construct
  source 1..126: note = MPH12 VH-FRGL-S63A VH (aa)
QVQLVESGGGLVKPGGSLRLSCAASGFTFSGFYMKWIRQAPGKGLEWVSSISASGSYTNYADAVEGRFTISRDNAKNSLYLQMNSLRAEDTAVYYCARGDSYCGASACHGEFFHKWGQGTLVTVSS

SEQ ID NO: 548 - protein, 17 aa, synthetic construct
  source 1..17: note = MPH12 VH-FRGL-S63A CDR-H2 (aa)
SISASGSYTNYADAVEG

SEQ ID NO: 549 - DNA, 401 bp, synthetic construct
  source 1..401: mol_type = other DNA; note = MPH12 VH-2CP (C111P, C112.2P) VH (co-nt)
accggtgtacattctcaggtgcagctggtggagagcgggggtggtctggtcaaacctggggggtcactgaggctgtcatgcgtggcaagcggattcactttttctggcttctacatgaagtggatcaggcaggctccaggcaagggcctggagtgggtgtccagcatcagcgcctctggctcctacaccaactatgctgactccgtggagggcaggttcacagtgtcccgggataacgctaagaatagcctgtttctgctgatgtcttccctgagagccgaggacaccgccgtgtactattgcgcccgcggcgatagctatcctggcgcttctgcacctcacggcgagttctttcacaaatgggggcagggcacactggtcactgtcagcagcgcgtcgac

SEQ ID NO: 550 - protein, 126 aa, synthetic construct
  source 1..126: note = MPH12 VH-2CP (C111P, C112.2P) VH (aa)
QVQLVESGGGLVKPGGSLRLSCVASGFTFSGFYMKWIRQAPGKGLEWVSSISASGSYTNYADSVEGRFTVSRDNAKNSLFLLMSSLRAEDTAVYYCARGDSYPGASAPHGEFFHKWGQGTLVTVSS

SEQ ID NO: 551 - protein, 19 aa, synthetic construct
  source 1..19: note = MPH12 VH-2CP (C111P, C112.2P) CDR-H3 (aa)
ARGDSYPGASAPHGEFFHK

SEQ ID NO: 552 - DNA, 392 bp, synthetic construct
  source 1..392: mol_type = other DNA; note = MPH12 VL (co-nt)
accggtgtacattctcagagcgtcctgacccagccccccagtgtgagcggagccccaggccagcgtgtgaccatttcttgtaccgggagcgatagcaacattggcgccgacttcgatgtgcactggtaccagcagctgccaggaaccgctcctaagctgctgatctatggcaacatcaatagaccatccggagtgccagacaggttctccggcagccggtctggcacctccgcctttctggctatcacacgcctgcaggccgaggacgaggctgattactattgccagtcttacgataggtctctgcattatgtcttcggagctggtacaaaggtcaccgtcctgggtcagcccaaggctgccccctcggtcactctgttcccgccctcgag

SEQ ID NO: 553 - protein, 110 aa, synthetic construct
  source 1..110: note = MPH12 VL (aa)
QSVLTQPPSVSGAPGQRVTISCTGSDSNIGADFDVHWYQQLPGTAPKLLIYGNINRPSGVPDRFSGSRSGTSAFLAITRLQAEDEADYYCQSYDRSLHYVFGAGTKVTVL

SEQ ID NO: 554 - protein, 9 aa, synthetic construct
  source 1..9: note = MPH12; MPH12 VL-FRGL CDR-L1 (aa)
DSNIGADFD

SEQ ID NO: 555 - skipped

SEQ ID NO: 556 - protein, 10 aa, synthetic construct
  source 1..10: note = MPH12; MPH12 VL-FRGL-D25E; MPH12 VL-FRGL-S26A; MPH12 VL-FRGL-D25S; MPH12 VL-S26A; MPH12 VL-D25E; MPH12 VL-D25S; MPH12 VL-FRGL CDR-L3 (aa)
QSYDRSLHYV

SEQ ID NO: 557 - DNA, 392 bp, synthetic construct
  source 1..392: mol_type = other DNA; note = MPH12 VL-FRGL VL (co-nt)
accggtgtacattctcagagcgtcctgacccagccccccagtgtgagcggagccccaggccagcgtgtgaccatttcttgtaccgggagcgatagcaacattggcgccgacttcgatgtgcactggtaccagcagctgccaggaaccgctcctaagctgctgatctatggcaacatcaatagaccatccggagtgccagacaggttctccggcagcaaatctggcacctccgccagcctggctatcacaggcctgcaggccgaggacgaggctgattactattgccagtcttacgataggtctctgcattatgtcttcggaaccggtacaaaggtcaccgtcctgggtcagcccaaggctgccccctcggtcactctgttcccgccctcgag

SEQ ID NO: 558 - protein, 110 aa, synthetic construct
  source 1..110: note = MPH12 VL-FRGL VL (aa)
QSVLTQPPSVSGAPGQRVTISCTGSDSNIGADFDVHWYQQLPGTAPKLLIYGNINRPSGVPDRFSGSKSGTSASLAITGLQAEDEADYYCQSYDRSLHYVFGTGTKVTVL

SEQ ID NO: 559 - DNA, 392 bp, synthetic construct
  source 1..392: mol_type = other DNA; note = MPH12 VL-D25S VL (co-nt)
accggtgtacattctcagagcgtcctgacccagccccccagtgtgagcggagccccaggccagcgtgtgaccatttcttgtaccgggagcagcagcaacattggcgccgacttcgatgtgcactggtaccagcagctgccaggaaccgctcctaagctgctgatctatggcaacatcaatagaccatccggagtgccagacaggttctccggcagccggtctggcacctccgcctttctggctatcacacgcctgcaggccgaggacgaggctgattactattgccagtcttacgataggtctctgcattatgtcttcggagctggtacaaaggtcaccgtcctgggtcagcccaaggctgccccctcggtcactctgttcccgccctcgag

SEQ ID NO: 560 - protein, 110 aa, synthetic construct
  source 1..110: note = MPH12 VL-D25S VL (aa)
QSVLTQPPSVSGAPGQRVTISCTGSSSNIGADFDVHWYQQLPGTAPKLLIYGNINRPSGVPDRFSGSRSGTSAFLAITRLQAEDEADYYCQSYDRSLHYVFGAGTKVTVL

SEQ ID NO: 561 - protein, 9 aa, synthetic construct
  source 1..9: note = MPH12 VL-D25S; MPH12 VL-FRGL-D25S CDR-L1 (aa)
SSNIGADFD

SEQ ID NO: 562 - DNA, 392 bp, synthetic construct
  source 1..392: mol_type = other DNA; note = MPH12 VL-D25E VL (co-nt)
accggtgtacattctcagagcgtcctgacccagccccccagtgtgagcggagccccaggccagcgtgtgaccatttcttgtaccgggagcgaaagcaacattggcgccgacttcgatgtgcactggtaccagcagctgccaggaaccgctcctaagctgctgatctatggcaacatcaatagaccatccggagtgccagacaggttctccggcagccggtctggcacctccgcctttctggctatcacacgcctgcaggccgaggacgaggctgattactattgccagtcttacgataggtctctgcattatgtcttcggagctggtacaaaggtcaccgtcctgggtcagcccaaggctgccccctcggtcactctgttcccgccctcgag

SEQ ID NO: 563 - protein, 110 aa, synthetic construct
  source 1..110: note = MPH12 VL-D25E VL (aa)
QSVLTQPPSVSGAPGQRVTISCTGSESNIGADFDVHWYQQLPGTAPKLLIYGNINRPSGVPDRFSGSRSGTSAFLAITRLQAEDEADYYCQSYDRSLHYVFGAGTKVTVL

SEQ ID NO: 564 - protein, 9 aa, synthetic construct
  source 1..9: note = MPH12 VL-D25E CDR-L1 (aa)
ESNIGADFD

SEQ ID NO: 565 - skipped

SEQ ID NO: 566 - DNA, 392 bp, synthetic construct
  source 1..392: mol_type = other DNA; note = MPH12 VL-S26A VL (co-nt)
accggtgtacattctcagagcgtcctgacccagccccccagtgtgagcggagccccaggccagcgtgtgaccatttcttgtaccgggagcgatgcgaacattggcgccgacttcgatgtgcactggtaccagcagctgccaggaaccgctcctaagctgctgatctatggcaacatcaatagaccatccggagtgccagacaggttctccggcagccggtctggcacctccgcctttctggctatcacacgcctgcaggccgaggacgaggctgattactattgccagtcttacgataggtctctgcattatgtcttcggagctggtacaaaggtcaccgtcctgggtcagcccaaggctgccccctcggtcactctgttcccgccctcgag

SEQ ID NO: 567 - protein, 110 aa, synthetic construct
  source 1..110: note = MPH12 VL-S26A VL (aa)
QSVLTQPPSVSGAPGQRVTISCTGSDANIGADFDVHWYQQLPGTAPKLLIYGNINRPSGVPDRFSGSRSGTSAFLAITRLQAEDEADYYCQSYDRSLHYVFGAGTKVTVL

SEQ ID NO: 568 - protein, 9 aa, synthetic construct
  source 1..9: note = MPH12 VL-S26A; MPH12 VL-FRGL-S26A CDR-L1 (aa)
DANIGADFD

SEQ ID NO: 569 - DNA, 392 bp, synthetic construct
  source 1..392: mol_type = other DNA; note = MPH12 VL-FRGL-D25S VL (co-nt)
accggtgtacattctcagagcgtcctgacccagccccccagtgtgagcggagccccaggccagcgtgtgaccatttcttgtaccgggagcagcagcaacattggcgccgacttcgatgtgcactggtaccagcagctgccaggaaccgctcctaagctgctgatctatggcaacatcaatagaccatccggagtgccagacaggttctccggcagcaaatctggcacctccgccagcctggctatcacaggcctgcaggccgaggacgaggctgattactattgccagtcttacgataggtctctgcattatgtcttcggaaccggtacaaaggtcaccgtcctgggtcagcccaaggctgccccctcggtcactctgttcccgccctcgag

SEQ ID NO: 570 - protein, 110 aa, synthetic construct
  source 1..110: note = MPH12 VL-FRGL-D25S VL (aa)
QSVLTQPPSVSGAPGQRVTISCTGSSSNIGADFDVHWYQQLPGTAPKLLIYGNINRPSGVPDRFSGSKSGTSASLAITGLQAEDEADYYCQSYDRSLHYVFGTGTKVTVL

SEQ ID NO: 571 - DNA, 392 bp, synthetic construct
  source 1..392: mol_type = other DNA; note = MPH12 VL-FRGL-D25E VL (co-nt)
accggtgtacattctcagagcgtcctgacccagccccccagtgtgagcggagccccaggccagcgtgtgaccatttcttgtaccgggagcgaaagcaacattggcgccgacttcgatgtgcactggtaccagcagctgccaggaaccgctcctaagctgctgatctatggcaacatcaatagaccatccggagtgccagacaggttctccggcagcaaatctggcacctccgccagcctggctatcacaggcctgcaggccgaggacgaggctgattactattgccagtcttacgataggtctctgcattatgtcttcggaaccggtacaaaggtcaccgtcctgggtcagcccaaggctgccccctcggtcactctgttcccgccctcgag

SEQ ID NO: 572 - protein, 110 aa, synthetic construct
  source 1..110: note = MPH12 VL-FRGL-D25E VL (aa)
QSVLTQPPSVSGAPGQRVTISCTGSESNIGADFDVHWYQQLPGTAPKLLIYGNINRPSGVPDRFSGSKSGTSASLAITGLQAEDEADYYCQSYDRSLHYVFGTGTKVTVL

SEQ ID NO: 573 - DNA, 392 bp, synthetic construct
  source 1..392: mol_type = other DNA; note = MPH12 VL-FRGL-S26A VL (co-nt)
accggtgtacattctcagagcgtcctgacccagccccccagtgtgagcggagccccaggccagcgtgtgaccatttcttgtaccgggagcgatgcgaacattggcgccgacttcgatgtgcactggtaccagcagctgccaggaaccgctcctaagctgctgatctatggcaacatcaatagaccatccggagtgccagacaggttctccggcagcaaatctggcacctccgccagcctggctatcacaggcctgcaggccgaggacgaggctgattactattgccagtcttacgataggtctctgcattatgtcttcggaaccggtacaaaggtcaccgtcctgggtcagcccaaggctgccccctcggtcactctgttcccgccctcgag

SEQ ID NO: 574 - protein, 110 aa, synthetic construct
  source 1..110: note = MPH12 VL-FRGL-S26A VL (aa)
QSVLTQPPSVSGAPGQRVTISCTGSDANIGADFDVHWYQQLPGTAPKLLIYGNINRPSGVPDRFSGSKSGTSASLAITGLQAEDEADYYCQSYDRSLHYVFGTGTKVTVL

SEQ ID NO: 575 - DNA, 381 bp, synthetic construct
  source 1..381: mol_type = other DNA; note = RSD5 VH (nt)
caggtgcagctggtggagtctgggggaggcgtggtccagcctgggaggcccctcagactctcctgtgcagcctctggattcgccttcaataattttgctttacactgggtccgccaggctccaggcaagggtccagagtggctggcagctgtgtcctatgacggaaccagtccatactacgcagagtccgtcagggcccgattcagcatctccagagacaattccaagaaaacattctatctgcaattggacagcctgcgacctgaagacacggctgtctattactgtgcgagagggcttggttcggggagttattcgtggattggttacttttatgcaatggacgtctggggccgagggacgacggtcaccgtctcctca

SEQ ID NO: 576 - protein, 127 aa, synthetic construct
  source 1..127: note = RSD5 VH (aa)
QVQLVESGGGVVQPGRPLRLSCAASGFAFNNFALHWVRQAPGKGPEWLAAVSYDGTSPYYAESVRARFSISRDNSKKTFYLQLDSLRPEDTAVYYCARGLGSGSYSWIGYFYAMDVWGRGTTVTVSS

SEQ ID NO: 577 - protein, 8 aa, synthetic construct
  source 1..8: note = RSD5-CDR-H1 (aa)
GFAFNNFA

SEQ ID NO: 578 - protein, 8 aa, synthetic construct
  source 1..8: note = RSD5-CDR-H2 (aa)
VSYDGTSP

SEQ ID NO: 579 - protein, 20 aa, synthetic construct
  source 1..20: note = RSD5-CDR-H3 (aa)
ARGLGSGSYSWIGYFYAMDV

SEQ ID NO: 580 - DNA, 330 bp, synthetic construct
  source 1..330: mol_type = other DNA; note = RSD5 VL (nt)
cagtctgccctgactcagcctgcctccgtgtctgggtctcctggacaatcgatcaccatctcctgcactggaaccagcagtgacgttggtggttatgtctatgtcgcctggtaccaacaacacccaggcacagcccccaaactcatcatttatgatgtcagtgatcggccctcaggggtttctaatcgattctctgggtccaagtctggcaacacggcctccctgaccatctctgggctccaggctgaggacgacgctgattattactgcatctcgtataccgacagaaacactgtcgtttttggcggcgggaccaagttgaccgtcctg

SEQ ID NO: 581 - protein, 110 aa, synthetic construct
  source 1..110: note = RSD5 VL (aa)
QSALTQPASVSGSPGQSITISCTGTSSDVGGYVYVAWYQQHPGTAPKLIIYDVSDRPSGVSNRFSGSKSGNTASLTISGLQAEDDADYYCISYTDRNTVVFGGGTKLTVL

SEQ ID NO: 582 - protein, 9 aa, synthetic construct
  source 1..9: note = RSD5-CD-L1 (aa)
SSDVGGYVY

SEQ ID NO: 583 - skipped

SEQ ID NO: 584 - protein, 10 aa, synthetic construct
  source 1..10: note = RSD5-CD-L3 (aa)
ISYTDRNTVV

SEQ ID NO: 585 - DNA, 381 bp, synthetic construct
  source 1..381: mol_type = other DNA; note = RSD5-FR-GL - VH (nt)
caggtgcagctggtcgagtctggaggaggagtggtccagccaggacggagtctgagactgtcatgcgccgctagcggcttcgcttttaacaatttcgcaatgcactgggtgcggcaggcaccaggcaagggactggaatgggtcgctgtggtcagttacgacgggacttcaccttactatgccgatagcgtgaagggcaggtttaccatctcccgcgacaactctaaaaatacactgtacctgcagatgaactccctgcgagcagaggataccgccgtgtactattgtgccagaggactgggcagcgggtcctattcttggatcggctacttctatgctatggatgtgtgggggcagggcactaccgtcactgtgtcttca

SEQ ID NO: 586 - protein, 127 aa, synthetic construct
  source 1..127: note = RSD5-FR-GL VH (aa)
QVQLVESGGGVVQPGRSLRLSCAASGFAFNNFAMHWVRQAPGKGLEWVAVVSYDGTSPYYADSVKGRFTISRDNSKNTLYLQMNSLRAEDTAVYYCARGLGSGSYSWIGYFYAMDVWGQGTTVTVSS

SEQ ID NO: 587 - DNA, 330 bp, synthetic construct
  source 1..330: mol_type = other DNA; note = RSD5-FR-GL VL (nt)
cagtccgcactgactcagccagcctctgtgagtggatcacccggccagagcatcacaatttcctgcaccggcacaagctccgacgtcggaggatacgtgtatgtctcttggtaccagcagcaccccggcaaggctcctaaactgatgatctatgatgtgagcaaccggcccagtggggtctcaaatagattcagcgggtccaagtctggaaacacagcctctctgactatcagtggcctgcaggccgaggacgaagctgattactattgtattagctacaccgacaggaatacagtggtctttggaggcgggactaaactgaccgtgctg

SEQ ID NO: 588 - protein, 110 aa, synthetic construct
  source 1..110: note = RSD5-FR-GL VL (aa)
QSALTQPASVSGSPGQSITISCTGTSSDVGGYVYVSWYQQHPGKAPKLMIYDVSNRPSGVSNRFSGSKSGNTASLTISGLQAEDEADYYCISYTDRNTVVFGGGTKLTVL

SEQ ID NO: 589 - skipped

SEQ ID NO: 590 - DNA, 384 bp, synthetic construct
  source 1..384: mol_type = other DNA; note = HMB2437 VH (nt)
caggtcaccttgaaggagtctggtcctgtgctggtgaaaccctcagagaccctcacgctgacctgcaccgtctctggattctcactcacagatgctagaatgggtgtgagttggatccgtcagcccccagggaaggccctggagtggcttgcacacattttctcgaatgacgaaaaattctacagcacatctctgaagaccaggctcaccatctccaaggacacctccacaagccaggtggtccttaggatgaccaacatggaccctgtggacacagccacttattattgtgcacgagtcgatcagggatgggtaaacacgtacagcgccttttattatggtatggacttctggggccaagggaccacggtcaccgtctcctca

SEQ ID NO: 591 - protein, 128 aa, synthetic construct
  source 1..128: note = HMB2437 VH (aa)
QVTLKESGPVLVKPSETLTLTCTVSGFSLTDARMGVSWIRQPPGKALEWLAHIFSNDEKFYSTSLKTRLTISKDTSTSQVVLRMTNMDPVDTATYYCARVDQGWVNTYSAFYYGMDFWGQGTTVTVSS

SEQ ID NO: 592 - protein, 10 aa, synthetic construct
  source 1..10: note = HMB2437-CDR-H1 (aa)
GFSLTDARMG

SEQ ID NO: 593 - protein, 7 aa, synthetic construct
  source 1..7: note = HMB2437-CDR-H2 (aa)
IFSNDEK

SEQ ID NO: 594 - protein, 20 aa, synthetic construct
  source 1..20: note = HMB2437-CDR-H3 (aa)
ARVDQGWVNTYSAFYYGMDF

SEQ ID NO: 595 - DNA, 321 bp, synthetic construct
  source 1..321: mol_type = other DNA; note = HMB2437 VL (nt)
gacatccagatgacccagtctccatcctccctgtctgcatctgtaggagaaagagtcaccatcacttgccgggcaagtcagtacattagcacccatttaaattggtatcagcacaaaccagggaaagcccctcgtctcctgatctatggtgcctcccatttggaaggtggggacccatcacggttcagtggcagtggatctgggacagatttcagtctcaccattaccagtctgcaacctgaagattttgcaacttactactgtcaacagacttataaaaccccgatcaccttcgcccaagggacacgactggagattaaa

SEQ ID NO: 596 - protein, 107 aa, synthetic construct
  source 1..107: note = HMB2437 VL (aa)
DIQMTQSPSSLSASVGERVTITCRASQYISTHLNWYQHKPGKAPRLLIYGASHLEGGDPSRFSGSGSGTDFSLTITSLQPEDFATYYCQQTYKTPITFAQGTRLEIK

SEQ ID NO: 597 - protein, 6 aa, synthetic construct
  source 1..6: note = HMB2437-CD-L1 (aa)
QYISTH

SEQ ID NO: 598 - protein, 9 aa, synthetic construct
  source 1..9: note = HMB2437-CD-L3 (aa)
QQTYKTPIT

SEQ ID NO: 599 - DNA, 384 bp, synthetic construct
  source 1..384: mol_type = other DNA; note = HMB2437_FR_GL__VH (nt)
caagtgactctgaaggagtctggcccagtgctggtcaaacccaccgaaacactgactctgacctgcacagtgagcggcttctccctgaccgacgctcggatgggggtctcctggatcagacagccacctggaaaggcactggagtggctggctcacatttttagcaacgacgaaaagagctactccacatctctgaaatctaggctgaccatcagtaaggatacaagtaaatcacaggtggtcctgactatgaccaacatggaccccgtggatacagcaacttactattgtgcccgggtggatcaggggtgggtcaatacctattcagccttctattatggaatggacttctggggacaggggactaccgtcaccgtctcttca

SEQ ID NO: 600 - protein, 128 aa, synthetic construct
  source 1..128: note = HMB2437-FR-GL VH (aa)
QVTLKESGPVLVKPTETLTLTCTVSGFSLTDARMGVSWIRQPPGKALEWLAHIFSNDEKSYSTSLKSRLTISKDTSKSQVVLTMTNMDPVDTATYYCARVDQGWVNTYSAFYYGMDFWGQGTTVTVSS

SEQ ID NO: 601 - DNA, 321 bp, synthetic construct
  source 1..321: mol_type = other DNA; note = HMB2437-FR-GL VL (nt)
gacatccagatgacccagagccctagctccctgtccgcatctgtgggggatcgggtcaccatcacatgcagagcctcccagtacatttctacacacctgaactggtatcagcagaagcccggcaaagcccctaagctgctgatctacggagcttctagtctgcagagtggcgtgccctctaggttcagtggctcagggagcggaacagactttactctgaccatttcaagcctgcagccagaggatttcgctacttactattgtcagcagacatataaaactcccatcacctttggccaggggacccggctggaaatcaaa

SEQ ID NO: 602 - protein, 107 aa, synthetic construct
  source 1..107: note = HMB2437-FR-GL VL (aa)
DIQMTQSPSSLSASVGDRVTITCRASQYISTHLNWYQQKPGKAPKLLIYGASSLQSGVPSRFSGSGSGTDFTLTISSLQPEDFATYYCQQTYKTPITFGQGTRLEIK

SEQ ID NO: 603 - DNA, 384 bp, synthetic construct
  source 1..384: mol_type = other DNA; note = HMB2432-v1 VH (nt)
gaggtgcagctggtgcagtctggagcggaggtgaaaaaacccgggacgtctctgaagatctcctgcaagggttctggatttagttttagcaactattggatcggctgggtgcgccagatgcccgggaaaggcctggagtggatggggatcgtctatccggctgactctgacaccagatacagcccgtccttccagggccaggtcaccatctcaggcgacaactccatcaataccgcctacctgcagtggagccgcctgaaggcctcggacaccgccacctattactgtgtgagacaaatcgggggggtggtgacaactgctactgacgactacttctacggtatggacatctggggcccagggaccacggtcatcgtctcctca

SEQ ID NO: 604 - protein, 128 aa, synthetic construct
  source 1..128: note = HMB2432-v1 VH (aa)
EVQLVQSGAEVKKPGTSLKISCKGSGFSFSNYWIGWVRQMPGKGLEWMGIVYPADSDTRYSPSFQGQVTISGDNSINTAYLQWSRLKASDTATYYCVRQIGGVVTTATDDYFYGMDIWGPGTTVIVSS

SEQ ID NO: 605 - protein, 8 aa, synthetic construct
  source 1..8: note = HMB2432-CDR-H1 (aa)
GFSFSNYW

SEQ ID NO: 606 - protein, 8 aa, synthetic construct
  source 1..8: note = HMB2432-CDR-H2 (aa)
VYPADSDT

SEQ ID NO: 607 - protein, 21 aa, synthetic construct
  source 1..21: note = HMB2432-CDR-H3 (aa)
VRQIGGVVTTATDDYFYGMDI

SEQ ID NO: 608 - DNA, 321 bp, synthetic construct
  source 1..321: mol_type = other DNA; note = HMB2432-v1 VL (nt)
gaaacgacactcacgcagtctccatcctccctgtctgcatctgtaggagacagagtcaccatcacttgccgggcaagtcagaccgttaaaagctttttaaattggtatcagcagaagccagggaaagcccctaaactcctgatctatgatgcatccgatttgcaaagtggggtcccatccaggttcagtggcagtggatctgggacagatttcactctcaccatcagccgtctgcaacctgaagattttgcaacttacttctgtcaacagagttacaggacccctctgacgttcggccaagggaccagggtggaaatcaaa

SEQ ID NO: 609 - protein, 107 aa, synthetic construct
  source 1..107: note = HMB2432-v1 VL (aa)
ETTLTQSPSSLSASVGDRVTITCRASQTVKSFLNWYQQKPGKAPKLLIYDASDLQSGVPSRFSGSGSGTDFTLTISRLQPEDFATYFCQQSYRTPLTFGQGTRVEIK

SEQ ID NO: 610 - protein, 6 aa, synthetic construct
  source 1..6: note = HMB2432-CD-L1 (aa)
QTVKSF

SEQ ID NO: 611 - protein, 9 aa, synthetic construct
  source 1..9: note = HMB2432-CD-L3 (aa)
QQSYRTPLT

SEQ ID NO: 612 - DNA, 384 bp, synthetic construct
  source 1..384: mol_type = other DNA; note = HMB2432-v2 VH (nt)
gaggtgcagctggtgcagtctggagcggaggtgaaaaaacccgggacgtctctgaagatctcctgcaagggttctggatttagttttagcaactattggatcggctgggtgcgccagatgcccgggaaaggcctggagtggatggggatcgtctatccggctgactctgacaccagatacagcccgtccttccagggccaggtcaccatctcaggcgacaactccatcaataccgcctacctgcagtggagccgcctgaaggcctcggacaccgccacctattactgtgtgagacaaatcgggggggtggtgacaactgctactgacgactacttctacggtatggacatctggggcccagggaccacggtcaccgtctcctca

SEQ ID NO: 613 - protein, 128 aa, synthetic construct
  source 1..128: note = HMB2432-v2 VH (aa)
EVQLVQSGAEVKKPGTSLKISCKGSGFSFSNYWIGWVRQMPGKGLEWMGIVYPADSDTRYSPSFQGQVTISGDNSINTAYLQWSRLKASDTATYYCVRQIGGVVTTATDDYFYGMDIWGPGTTVTVSS

SEQ ID NO: 614 - DNA, 321 bp, synthetic construct
  source 1..321: mol_type = other DNA; note = HMB2432-v2 VL (nt)
gaaacgacactcacgcagtctccatcctccctgtctgcatctgtaggagacagagtcaccatcacttgccgggcaagtcagaccgttaaaagctttttaaattggtatcagcagaagccagggaaagcccctaaactcctgatctatgatgcatccgatttgcaaagtggggtcccatccaggttcagtggcagtggatctgggacagatttcactctcaccatcagccgtctgcaacctgaagattttgcaacttacttctgtcaacagagttacaggacccctctgacgttcggccaagggaccaaggtggaaatcaaa

SEQ ID NO: 615 - protein, 107 aa, synthetic construct
  source 1..107: note = HMB2432-v2 VL (aa)
ETTLTQSPSSLSASVGDRVTITCRASQTVKSFLNWYQQKPGKAPKLLIYDASDLQSGVPSRFSGSGSGTDFTLTISRLQPEDFATYFCQQSYRTPLTFGQGTKVEIK

SEQ ID NO: 616 - DNA, 369 bp, synthetic construct
  source 1..369: mol_type = other DNA; note = HMB2416 VH (nt)
caggtgcagctgcaggagtcgggcccaggactcgtgaagccttcacagaccctgtccctcacctgcactgtctctggtggctccatcagcagtggtgattactactggagttggatccgccagcccccagggaagggcctggagtggattgggtacatctacttcagtggcagcacctactacaatccgtccctcaagagtcgagttaccatgtcaggggacacgtccaagaatcagttctccctgaggctgagctctgtgactgccgcagacacggccgtgtattattgtgccagagaggatacaactatggctattccatactacttcgacccctggggccggggaatcctggtcaccgtctcctca

SEQ ID NO: 617 - protein, 123 aa, synthetic construct
  source 1..123: note = HMB2416 VH (aa)
QVQLQESGPGLVKPSQTLSLTCTVSGGSISSGDYYWSWIRQPPGKGLEWIGYIYFSGSTYYNPSLKSRVTMSGDTSKNQFSLRLSSVTAADTAVYYCAREDTTMAIPYYFDPWGRGILVTVSS

SEQ ID NO: 618 - protein, 10 aa, synthetic construct
  source 1..10: note = HMB2416-CDR-H1 (aa)
GGSISSGDYY

SEQ ID NO: 619 - protein, 7 aa, synthetic construct
  source 1..7: note = HMB2416-CDR-H2 (aa)
IYFSGST

SEQ ID NO: 620 - protein, 15 aa, synthetic construct
  source 1..15: note = HMB2416-CDR-H3 (aa)
AREDTTMAIPYYFDP

SEQ ID NO: 621 - DNA, 324 bp, synthetic construct
  source 1..324: mol_type = other DNA; note = HMB2416 VL (nt)
tcctatgtcctgactcagccaccctcagtgtcagtggcccccggaaggacggccaggatcacctgtgggagacataatattggaaatgaaaatgttcactggtaccagcagaggccaggccaggcccctgtgctggtcatctattctgatagcgaccggccctcagggatccctgagcgattctctggctccaactctgggaacacggccaccctaagcatcagcagggtcgaagccggggatgaggccgactattattgtcaggtgtgggatagtagtactgatcaagtggtattcggcggagggaccaagctgaccgtccta

SEQ ID NO: 622 - protein, 108 aa, synthetic construct
  source 1..108: note = HMB2416 VL (aa)
SYVLTQPPSVSVAPGRTARITCGRHNIGNENVHWYQQRPGQAPVLVIYSDSDRPSGIPERFSGSNSGNTATLSISRVEAGDEADYYCQVWDSSTDQVVFGGGTKLTVL

SEQ ID NO: 623 - protein, 6 aa, synthetic construct
  source 1..6: note = HMB2416-CD-L1 (aa)
NIGNEN

SEQ ID NO: 624 - skipped

SEQ ID NO: 625 - protein, 11 aa, synthetic construct
  source 1..11: note = HMB2416-CD-L3 (aa)
QVWDSSTDQVV

SEQ ID NO: 626 - DNA, 373 bp, synthetic construct
  source 1..373: mol_type = other DNA; note = HMB3210 VH (nt)
gaggaacagctgctagagtctgggggaggcctggtcaagcctggggggtccctgagactctcctgtgcagcctctggattcaccttcagtagttatagcatgaactgggtccgccaggctccagggaaggggctggagtgggtctcatccattagtgcaagtagcagttacagcgattacgcagactcagcgaagggccgattcaccatctccagagacaacgccaagacctcactgtttctgcaaatgaacagcctgagagccgaggacacggctatctatttctgtgcgagagctcgggcaactggctacagttccattaccccctactttgacatttggggccagggaaccctggtcaccgtctcctcag

SEQ ID NO: 627 - protein, 124 aa, synthetic construct
  source 1..124: note = HMB3210 VH (aa)
EEQLLESGGGLVKPGGSLRLSCAASGFTFSSYSMNWVRQAPGKGLEWVSSISASSSYSDYADSAKGRFTISRDNAKTSLFLQMNSLRAEDTAIYFCARARATGYSSITPYFDIWGQGTLVTVSS

SEQ ID NO: 628 - protein, 8 aa, synthetic construct
  source 1..8: note = HMB3210-CDR-H1 (aa)
GFTFSSYS

SEQ ID NO: 629 - protein, 8 aa, synthetic construct
  source 1..8: note = HMB3210-CDR-H2 (aa)
ISASSSYS

SEQ ID NO: 630 - protein, 17 aa, synthetic construct
  source 1..17: note = HMB3210-CDR-H3 (aa)
ARARATGYSSITPYFDI

SEQ ID NO: 631 - DNA, 331 bp, synthetic construct
  source 1..331: mol_type = other DNA; note = HMB3210 VL (nt)
cagtctgtcgtgacgcagacgccctcagtgtctggggccccagggcagagggtcaccatctcctgcactgggagcagctccaacatcggggcaggttatgatgtacactggtaccagcaacttccaggaacagcccccaaactcctcatctatgataacaacaatcgaccctcaggggtcccggaccgattctctgcctccaagtctggcacctcagcctccctggccatcaccgggctccaggctgaggatgaggctgattattactgccagtcctatgacaggaacctgagtggtgtcttcggaactgggaccaaggtcaccgtcctag

SEQ ID NO: 632 - protein, 110 aa, synthetic construct
  source 1..110: note = HMB3210 VL (aa)
QSVVTQTPSVSGAPGQRVTISCTGSSSNIGAGYDVHWYQQLPGTAPKLLIYDNNNRPSGVPDRFSASKSGTSASLAITGLQAEDEADYYCQSYDRNLSGVFGTGTKVTVL

SEQ ID NO: 633 - skipped

SEQ ID NO: 634 - protein, 10 aa, synthetic construct
  source 1..10: note = HMB3210-CD-L3 (aa)
QSYDRNLSGV

SEQ ID NO: 635 - DNA, 373 bp, synthetic construct
  source 1..373: mol_type = other DNA; note = HMB3210-v2 VH (nt)
gaggtgcagctggtggagtctgggggaggcctggtcaagcctggggggtccctgagactctcctgtgcagcctctggattcaccttcagtagttatagcatgaactgggtccgccaggctccagggaaggggctggagtgggtctcatccattagtgcaagtagcagttacagcgattacgcagactcagcgaagggccgattcaccatctccagagacaacgccaagacctcactgtttctgcaaatgaacagcctgagagccgaggacacggctatctatttctgtgcgagagctcgggcaactggctacagttccattaccccctactttgacatttggggccagggaaccctggtcaccgtctcctcag

SEQ ID NO: 636 - protein, 124 aa, synthetic construct
  source 1..124: note = HMB3210-v2 VH (aa)
EVQLVESGGGLVKPGGSLRLSCAASGFTFSSYSMNWVRQAPGKGLEWVSSISASSSYSDYADSAKGRFTISRDNAKTSLFLQMNSLRAEDTAIYFCARARATGYSSITPYFDIWGQGTLVTVSS

SEQ ID NO: 637 - DNA, 331 bp, synthetic construct
  source 1..331: mol_type = other DNA; note = HMB3210-v3 VL (nt)
cagtctgtcgtgacgcagccgccctcagtgtctggggccccagggcagagggtcaccatctcctgcactgggagcagctccaacatcggggcaggttatgatgtacactggtaccagcaacttccaggaacagcccccaaactcctcatctatgataacaacaatcgaccctcaggggtcccggaccgattctctgcctccaagtctggcacctcagcctccctggccatcaccgggctccaggctgaggatgaggctgattattactgccagtcctatgacaggagcctgagtggtgtcttcggaactgggaccaaggtcaccgtcctag

SEQ ID NO: 638 - protein, 110 aa, synthetic construct
  source 1..110: note = HMB3210-v3 VL (aa)
QSVVTQPPSVSGAPGQRVTISCTGSSSNIGAGYDVHWYQQLPGTAPKLLIYDNNNRPSGVPDRFSASKSGTSASLAITGLQAEDEADYYCQSYDRSLSGVFGTGTKVTVL

SEQ ID NO: 639 - protein, 10 aa, synthetic construct
  source 1..10: note = HMB3210-v3-CDRL3 (aa)
QSYDRSLSGV

SEQ ID NO: 640 - DNA, 372 bp, synthetic construct
  source 1..372: mol_type = other DNA; note = HMB3210-v4 VH (nt)
gaggtgcagctggtggagagcggaggcggactggtcaaacctggcgggtcactgagactgtcatgcgcagcaagcggcttcacattcagctcctactctatgaactgggtgcgacaggctcctggcaagggactggagtgggtctctagtatctcaagctcctctagttacatctactatgcagactccgtgaagggaaggttcaccatctcacgcgataacgccaaaaatagcctgtatctgcagatgaattccctgagagccgaagacaccgctgtctactattgcgcccgggctagagcaacaggctataacagcattactccttactttgacatctggggacagggcacactggtgaccgtctcctca

SEQ ID NO: 641 - protein, 124 aa, synthetic construct
  source 1..124: note = HMB3210-v4 VH (aa)
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARARATGYNSITPYFDIWGQGTLVTVSS

SEQ ID NO: 642 - protein, 8 aa, synthetic construct
  source 1..8: note = HMB3210-v4-CDRH2 (aa)
ISSSSSYI

SEQ ID NO: 643 - protein, 17 aa, synthetic construct
  source 1..17: note = HMB3210-v4-CDRH3 (aa)
ARARATGYNSITPYFDI

SEQ ID NO: 644 - DNA, 330 bp, synthetic construct
  source 1..330: mol_type = other DNA; note = HMB3210-v4 VL (nt)
cagtccgtcgtcactcagcctccaagcgtcagcggggcacctgggcagcgggtcacaatctcatgcactgggtcctcatccaacatcggcgctgggtacgacgtgcactggtatcagcagctgcctggaacagcacctaagctgctgatctacgggaacagcaatcggccatctggagtccccgatagattcagcggatccaaatctggcaccagtgcctcactggctattacagggctgcaggcagaggacgaagccgattactattgccagtcttatgattcttctctgtctggagtcttcggcaccggcacaaaagtcaccgtcctg

SEQ ID NO: 645 - protein, 110 aa, synthetic construct
  source 1..110: note = HMB3210-v4 VL (aa)
QSVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGVFGTGTKVTVL

SEQ ID NO: 646 - protein, 10 aa, synthetic construct
  source 1..10: note = HMB3210-v4-CDRL3 (aa)
QSYDSSLSGV

SEQ ID NO: 647 - DNA, 367 bp, synthetic construct
  source 1..367: mol_type = other DNA; note = HMB2430 VH (nt)
gaggtgcacctggtggagtctgggggaggcctggtcaagcctggggggtccctgagactctcctgtgcagcctctggattcgcattcactggttatggtctaaattgggtccgccaggttccagggaagggcctggagtgggtttcatccatcactgctggaagctcatacatcgactacgcagagtcagtgaagggccgattcaccatctccagagacaacggcaagaatacactgttcctgcaaatgagcgacctgagagccgacgacacggctgtctattactgtgcgagagttgcgtctcctctggttcggggactccacttagactactggggccagggagccctggtcaccgtctcctcag

SEQ ID NO: 648 - protein, 122 aa, synthetic construct
  source 1..122: note = HMB2430 VH (aa)
EVHLVESGGGLVKPGGSLRLSCAASGFAFTGYGLNWVRQVPGKGLEWVSSITAGSSYIDYAESVKGRFTISRDNGKNTLFLQMSDLRADDTAVYYCARVASPLVRGLHLDYWGQGALVTVSS

SEQ ID NO: 649 - protein, 8 aa, synthetic construct
  source 1..8: note = HMB2430-CDR-H1 (aa)
GFAFTGYG

SEQ ID NO: 650 - protein, 8 aa, synthetic construct
  source 1..8: note = HMB2430-CDR-H2 (aa)
ITAGSSYI

SEQ ID NO: 651 - protein, 15 aa, synthetic construct
  source 1..15: note = HMB2430-CDR-H3 (aa)
ARVASPLVRGLHLDY

SEQ ID NO: 652 - DNA, 331 bp, synthetic construct
  source 1..331: mol_type = other DNA; note = HMB2430 VL (nt)
cagtctgtgctgacgcagccgccctcaatgtccggggccccagggcagagggtcaccatctcctgcactgggggcagctccaacatcggggcaggttatgatgtgcagtggtaccagcaacttccaggagcagcccccaaactcctcatctatgctaacgacaatcggccctcaggggtccctgaccgattctctggctccaagtctggcacctcaggctccctagtcatcgctggcctccgggctgaggatgaggctgattattactgccagtcctatgaccgcaccctgagtgtagtgttcggcggagggaccaagctgaccgtcctgg

SEQ ID NO: 653 - protein, 110 aa, synthetic construct
  source 1..110: note = HMB2430 VL (aa)
QSVLTQPPSMSGAPGQRVTISCTGGSSNIGAGYDVQWYQQLPGAAPKLLIYANDNRPSGVPDRFSGSKSGTSGSLVIAGLRAEDEADYYCQSYDRTLSVVFGGGTKLTVL

SEQ ID NO: 654 - skipped

SEQ ID NO: 655 - protein, 10 aa, synthetic construct
  source 1..10: note = HMB2430-CD-L3 (aa)
QSYDRTLSVV

SEQ ID NO: 656 - DNA, 367 bp, synthetic construct
  source 1..367: mol_type = other DNA; note = HMB2430-v2 VH (nt)
gaggtgcagctggtggagtctgggggaggcctggtcaagcctggggggtccctgagactctcctgtgcagcctctggattcgcattcactggttatggtctaaattgggtccgccaggttccagggaagggcctggagtgggtttcatccatcactgctggaagctcatacatcgactacgcagagtcagtgaagggccgattcaccatctccagagacaacggcaagaatacactgttcctgcaaatgagcgacctgagagccgacgacacggctgtctattactgtgcgagagttgcgtctcctctggttcggggactccacttagactactggggccagggagccctggtcaccgtctcctcag

SEQ ID NO: 657 - protein, 122 aa, synthetic construct
  source 1..122: note = HMB2430-v2 VH (aa)
EVQLVESGGGLVKPGGSLRLSCAASGFAFTGYGLNWVRQVPGKGLEWVSSITAGSSYIDYAESVKGRFTISRDNGKNTLFLQMSDLRADDTAVYYCARVASPLVRGLHLDYWGQGALVTVSS

SEQ ID NO: 658 - DNA, 366 bp, synthetic construct
  source 1..366: mol_type = other DNA; note = HMB2430-v3 VH (nt)
gaagtgcagctggtggaatctgggggcgggctggtcaaacctggcggaagtctgaggctgtcctgtgctgctagtggctttacctttagctcctactctatgaactgggtgcgacaggcacctggcaagggactggagtgggtctctagtatctcaagctcctctagttacatctactatgctgactccgtgaagggccggttcaccatctcaagagataacgcaaaaaatagcctgtatctgcagatgaattccctgagggcagaagacacagccgtgtactattgcgcccgcgtcgctagccctatggtgcgggggctgcattttgattattggggacagggaactctggtgaccgtctcatcc

SEQ ID NO: 659 - protein, 122 aa, synthetic construct
  source 1..122: note = HMB2430-v3 VH (aa)
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARVASPMVRGLHFDYWGQGTLVTVSS

SEQ ID NO: 660 - protein, 15 aa, synthetic construct
  source 1..15: note = HMB2430-v3-CDRH3 (aa)
ARVASPMVRGLHFDY

SEQ ID NO: 661 - DNA, 330 bp, synthetic construct
  source 1..330: mol_type = other DNA; note = HMB2430-v3 VL (nt)
cagagcgtcctgacccagccaccatccgtgagcggcgcacccggccagcgagtgactatttcctgtaccggcagttcttcaaacatcggcgctgggtacgacgtgcactggtatcagcagctgcctggaacagcacctaagctgctgatctacgggaacagcaatcggccatctggagtccccgatagattcagcggatccaaatctggcaccagtgcctcactggctattacagggctgcaggcagaggacgaagccgattactattgccagagctacgattcatccctgagcgtggtcttcggaggcggcacaaaactgactgtcctg

SEQ ID NO: 662 - protein, 110 aa, synthetic construct
  source 1..110: note = HMB2430-v3 VL (aa)
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSVVFGGGTKLTVL

SEQ ID NO: 663 - protein, 10 aa, synthetic construct
  source 1..10: note = HMB2430-v3-CDRL3 (aa)
QSYDSSLSVV

SEQ ID NO: 664 - protein, 330 aa, Homo sapiens [sequence omitted]

SEQ ID NO: 665 - protein, 232 aa, Homo sapiens
  source 1..232: note = Wild-type human IgG1 hinge-CH2-CH3
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 666 - protein, 110 aa, Homo sapiens
  source 1..110: note = Wild-type human IgG1 CH2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 667 - protein, 107 aa, Homo sapiens
  source 1..107: note = Wild-type human IgG1 CH3
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 668 - protein, 125 aa, Homo sapiens
  source 1..125: note = Wild-type human IgG1 hinge-CH2
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 669 - protein, 330 aa, synthetic construct
  source 1..330: note = IgG1m3 CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 670 - protein, 330 aa, synthetic construct [sequence omitted]

SEQ ID NO: 671 - protein, 330 aa, synthetic construct
  source 1..330: note = IgG1m3,1 CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 672 - protein, 330 aa, synthetic construct
  source 1..330: note = Human IgG1 CH1_CH3 __ GAALIE mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 673 - protein, 232 aa, synthetic construct
  source 1..232: note = Human IgG1 hinge-CH2-CH3 - GAALIE mutation
EPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 674 - protein, 110 aa, synthetic construct
  source 1..110: note = Human IgG1 CH2 - GAALIE mutation
APELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAK

SEQ ID NO: 675 - protein, 125 aa, synthetic construct
  source 1..125: note = Human IgG1 hinge-CH2; - GAALIE mutation
EPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAK

SEQ ID NO: 676 - protein, 330 aa, synthetic construct
  source 1..330: note = IgG1m3 CH1-CH3 - GAALIE mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 677 - protein, 330 aa, synthetic construct; identical to SEQ ID NO: 672
  source 1..330: note = IgG1m17,1 CH1-CH3- GAALIE mutation

SEQ ID NO: 678 - protein, 330 aa, synthetic construct
  source 1..330: note = IgG1m3,1 CH1-CH3 - GAALIE mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 679 - protein, 330 aa, synthetic construct
  source 1..330: note = Human IgG1 CH1-CH3_LS mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 680 - protein, 232 aa, synthetic construct
  source 1..232: note = Human IgG1 hinge-CH2-CH3_LS mutation
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 681 - protein, 107 aa, synthetic construct
  source 1..107: note = Human IgG1 CH3_LS mutation
GQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 682 - protein, 330 aa, synthetic construct
  source 1..330: note = IgG1m3 CH1-CH3_LS mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 683 - protein, 330 aa, synthetic construct; identical to SEQ ID NO: 679
  source 1..330: note = IgG1m17,1 CH1-CH3_LS mutation

SEQ ID NO: 684 - protein, 330 aa, synthetic construct
  source 1..330: note = IgG1m3,1 CH1-CH3_LS mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 685 - protein, 330 aa, synthetic construct
  source 1..330: note = Human IgG1 CH1-CH3_LA mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 686 - protein, 232 aa, synthetic construct
  source 1..232: note = Human IgG1 hinge-CH2-CH3_LA mutation
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 687 - protein, 107 aa, synthetic construct
  source 1..107: note = Human IgG1 CH3_LA mutation
GQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 688 - protein, 330 aa, synthetic construct
  source 1..330: note = IgG1m3 CH1-CH3_LS mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 689 - protein, 330 aa, synthetic construct; identical to SEQ ID NO: 685
  source 1..330: note = IgG1m17,1 CH1-CH3_LS mutation

SEQ ID NO: 690 - protein, 330 aa, synthetic construct
  source 1..330: note = IgG1m3,1 CH1-CH3_LS mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 691 - protein, 330 aa, synthetic construct
  source 1..330: note = Human IgG1 CH1-CH3_GAALIE and LS mutations
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 692 - protein, 232 aa, synthetic construct
  source 1..232: note = Human IgG1 hinge-CH2-CH3_GAALIE and LS mutations
EPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 693 - protein, 330 aa, synthetic construct
  source 1..330: note = IgG1m3 CH1-CH3_GAALIE and LS mutations
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 694 - protein, 330 aa, synthetic construct; identical to SEQ ID NO: 691
  source 1..330: note = IgG1m17,1 CH1-CH3_GAALIE and LS mutations

SEQ ID NO: 695 - protein, 330 aa, synthetic construct
  source 1..330: note = IgG1m3,1 CH1-CH3_GAALIE and LS mutations
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 696 - protein, 330 aa, synthetic construct
  source 1..330: note = Human IgG1 CH1-CH3_GAALIE and LA mutations
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 697 - protein, 232 aa, synthetic construct
  source 1..232: note = Human IgG1 hinge-CH2-CH3_GAALIE and LA mutations
EPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 698 - protein, 330 aa, synthetic construct
  source 1..330: note = IgG1m3 CH1-CH3_GAALIE and LA mutations
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 699 - protein, 330 aa, synthetic construct; identical to SEQ ID NO: 696
  source 1..330: note = IgG1m17,1 CH1-CH3_GAALIE and LA mutations

SEQ ID NO: 700 - protein, 330 aa, synthetic construct
  source 1..330: note = IgG1m3,1 CH1-CH3_GAALIE and LA mutations
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 701 - DNA, 357 bp, synthetic construct
  source 1..357: mol_type = other DNA; note = MPK190-v1.1 VH (nt) and MPK190-v1.3 VH (nt)
caggtgcagctggtggagtctggaggaggactggtgaagccaggaggatctctgaccctgtcctgcgccgctagcggcttcagcatctctggctacggcatgaactgggtgaggcaggctccaggaaggggactggagtgggtggcctccatctccgcctccagcacctacatctactatgctgactccatgaagggcagattcaccatctctcgcgataatggcaagacatccctgtatctgcagatgaagagcctgagggccgaggacacagccgtgtactattgtgccagagctcccatcgagcccccttattttgatctgtggggcaggggcaccctggtgacagtgtcttcc

SEQ ID NO: 702 - protein, 119 aa, synthetic construct
  source 1..119: note = MPK190-v1.1 VH (aa) and MPK190-v1.3 VH (aa)
QVQLVESGGGLVKPGGSLTLSCAASGFSISGYGMNWVRQAPGRGLEWVASISASSTYIYYADSMKGRFTISRDNGKTSLYLQMKSLRAEDTAVYYCARAPIEPPYFDLWGRGTLVTVSS

SEQ ID NO: 703 - DNA, 333 bp, synthetic construct
  source 1..333: mol_type = other DNA; note = MPK190-v1.3 VL (nt)
cagtccgtgctgacccagccaccttccgtgagcggagctccaggccagagggtgaccatcagctgcacaggctccagctctaacatcggcgccggctacgacgtgcactggtatcagcatctgccaggcaaggctcccaaggtgctgatctacggcaacacaaatagaccttctgtggtgccagatcgcttctctggctccaatagcggcgcctctgcttccctggctatctccggactgcaggctgaggacgaggctgattacttctgtcagagctatgacaggcggctgtctggccctatctttggcggcggcaccaagctgacagtgctg

SEQ ID NO: 704 - protein, 111 aa, synthetic construct
  source 1..111: note = MPK190-v1.3 VL (aa)
QSVLTQPPSVSGAPGQRVTISCTGSSSNIGAGYDVHWYQHLPGKAPKVLIYGNTNRPSVVPDRFSGSNSGASASLAISGLQAEDEADYFCQSYDRRLSGPIFGGGTKLTVL

SEQ ID NO: 705 - skipped

SEQ ID NO: 706 - DNA, 357 bp, synthetic construct
  source 1..357: mol_type = other DNA; note = MPK104-v1.1 VH (nt) and MPK104-v1.3 VH (nt)
caggtgcagctggtggagtctggaggaggactggtgaagccaggaggatccctgaggctgagctgcgccgcttctggcttcaccatcgacggctacggcatgaactgggtgagacaggctccaggcaagggactggagtgggtgtccagcatctccgcctcttccaattacatctactatgctgacagcatgaagggcaggttcaccatctcccgggataacggcaagaatagcctgtatctgcagatgcactctctgagagtggaggacacaggcgtgtactattgtgctagggctccactggagccaccttattttgatctgtggggcaggggcaccctggtgacagtgagctct

SEQ ID NO: 707 - protein, 119 aa, synthetic construct
  source 1..119: note = MPK104-v1.1 VH (aa) and MPK104-v1.3 VH (nt)
QVQLVESGGGLVKPGGSLRLSCAASGFTIDGYGMNWVRQAPGKGLEWVSSISASSNYIYYADSMKGRFTISRDNGKNSLYLQMHSLRVEDTGVYYCARAPLEPPYFDLWGRGTLVTVSS

SEQ ID NO: 708 - DNA, 333 bp, synthetic construct
  source 1..333: mol_type = other DNA; note = MPK104-v1.1 VL (nt)
cagtccgtgctgacccagccaccttccgtgtccggagctccaggccagagggtgacaatcagctgctctggcggctccagcaatatcggcgctggctacgacgtgcactggtatcagcaggtgccaggcaagggccccaaggtgctgatctacggcaacaataacagacctagcggcgtgccagatcgcttctccggcagcaactttggagcttctgcttccctggctatcaccggcctgcaggctgacgatgaggctgactacttctgtcagtcttatgataggcggctgggaggccccgtgttcggaggaggcaccaagctgacagtgctg

SEQ ID NO: 709 - DNA, 333 bp, synthetic construct
  source 1..333: mol_type = other DNA; note = MPK104-v1.3 VL (nt)
cagtccgtgctgacccagccaccttccgtgtccggagctccaggccagagggtgacaatcagctgctctggcggctccagcaacatcggcgctggctacgacgtgcactggtatcagcaggtgccaggcaagggccccaaggtgctgatctacggcaacaccaatagacctagcggcgtgccagatcgcttctccggcagcaattttggcgcctctgcttccctggctatcacaggcctgcaggctgaggacgaggctgattacttctgtcagtcttatgacaggcggctgggaggccccgtgttcggaggaggcaccaagctgacagtgctg

SEQ ID NO: 710 - protein, 111 aa, synthetic construct
  source 1..111: note = MPK104-v1.3 VL (aa)
QSVLTQPPSVSGAPGQRVTISCSGGSSNIGAGYDVHWYQQVPGKGPKVLIYGNTNRPSGVPDRFSGSNFGASASLAITGLQAEDEADYFCQSYDRRLGGPVFGGGTKLTVL

SEQ ID NO: 711 - DNA, 357 bp, synthetic construct
  source 1..357: mol_type = other DNA; note = MPK77-v1.1 VH (nt)
caggtgcagctggtggagagcggaggaggactggtgaagccaggaggaagcctgaggctgtcttgcgccgctagcaagtctttctccggctactatatgatctggatcagacaggctccaggaaggggactggagtggatcgctgacatctctggctccagcacctacacagactatgccgattccgtgaagggcaggtttaccatcagcagggacaacacccggaatacactgtacctgcagatgaactctctgagacccgaggataccgccgtgtactattgtgctacagagcaggtgccctcccctggcccattcgatctgtggggccctggcaccctggtgacagtgtcttcc

SEQ ID NO: 712 - protein, 119 aa, synthetic construct
  source 1..119: note = MPK77-v1.1 VH (aa)
QVQLVESGGGLVKPGGSLRLSCAASKSFSGYYMIWIRQAPGRGLEWIADISGSSTYTDYADSVKGRFTISRDNTRNTLYLQMNSLRPEDTAVYYCATEQVPSPGPFDLWGPGTLVTVSS

SEQ ID NO: 713 - DNA, 333 bp, synthetic construct
  source 1..333: mol_type = other DNA; note = MPK77-v1.1 VL (nt)
cagtctgtgctgacccagccaccttccgtgagcggagctccaggccagagagtgatcatctcctgcacaggctccagctctaacatcggcgctggctacgacgtgcactggtatcagcagttcccaggcaccagccccaagctgctgatctacaggaacacaaatcggccttccggcgtgccagatagattcagcggctttaagagcggagcttctgccgctctggctatcaccggcctgcaggctgaggacgaggctgattactattgtcagtcttatgaccgctctctgtccggatgggcctttggcggaggaaccaaggtgacagtgctg

SEQ ID NO: 714 - DNA, 333 bp, synthetic construct
  source 1..333: mol_type = other DNA; note = MPK190-v1.1 VL (nt)
cagtctgtgctgacccagccaccttccgtgagcggagctccaggccagagggtgaccatctcctgcacaggctctaattccaacatcggcgccggctacgacgtgcactggtatcagcatctgccaggcaaggctcccaaggtgctgatctacggcaacaataacagacctagcgtggtgccagatcgcttcagcggctctaattccggcgcctccgcctctctggctatctctggcctgcaggccgaggacgaggctgattacttctgtcagtcctatgacaggcggctgagcggacctatctttggaggaggcaccaagctgacagtgctg

SEQ ID NO: 715 - DNA, 360 bp, synthetic construct
  source 1..360: mol_type = other DNA; note = MPK51-v1.1 VH (nt)
caggtgcagctggtggagtccggaggaggactggtgaagccaggaggatccctgagactgagctgcgccgcttctggcttccgcatcagcggctactatatgatctgggtgaggcaggctcctggcaagggactggagtggatctccgccatctctggcatgtccagctacatcacctatgccgactctgtgaagggcaggttcacagtgtcccgggataacggccagaatagcctgtttctggagatgaagtctctgaggggcgaggacaccgccgtgtacttctgtgctagagaggaggtgccctcccctggcccatttgatctgtggggccagggcaccccagtgacagtgtcttcc

SEQ ID NO: 716 - protein, 120 aa, synthetic construct
  source 1..120: note = MPK51-v1.1 VH (aa)
QVQLVESGGGLVKPGGSLRLSCAASGFRISGYYMIWVRQAPGKGLEWISAISGMSSYITYADSVKGRFTVSRDNGQNSLFLEMKSLRGEDTAVYFCAREEVPSPGPFDLWGQGTPVTVSS

SEQ ID NO: 717 - DNA, 333 bp, synthetic construct
  source 1..333: mol_type = other DNA; note = MPK51-v1.1 VL (nt)
cagtccgtggtgacccagccaccttccgtgagcggagctccaggccagagggtgacaatcttctgcaccggctctacatccaatatcggcgccggctacgacgtgcactggtatcagcatctgccaggcaccgctcccagactgctgatctttaggaacaataaccggccttctggcgtgccagatcgcttctccggctttaagagcggcacaagcgcctctctggctatcatcggcctgcaggtggaggacgaggccgattactattgtcagtcctacgacagaaacatggatggatgggctttcggcggaggaaccaagctgacagtgctg

SEQ ID NO: 718 - DNA, 1347 bp, synthetic construct
  source 1..1347: mol_type = other DNA; note = MPK190_G1m3-LS VH (nt)
gaggtgcagctggtggagtcggggggaggcctggtcaagccgggggggtccctgacactctcctgtgcagcctctggattctccatcagtggatatggcatgaactgggtccgccaggctccagggaggggactggagtgggtcgcatccattagtgccagtagtacttacatatattatgcagactcaatgaagggccgattcaccatctccagagacaacggcaagacttcactctatctgcaaatgaagagtttgcgagccgaggacacggctgtctattattgtgcgagagcgcctatagaaccgccgtacttcgatctctggggccgtggcaccctggtcactgtctcctcagcgtcgaccaagggcccatcggtcttccccctggcaccaagtagcaagagcacatccggtggcacagccgccctgggttgtctggtgaaagattatttccctgagcccgtgacagtctcctggaactctggcgccctgacctccggagtgcacacattccctgctgtgctgcagtccagcggcctgtactccctgtcttccgtggtgaccgtgccaagctcttccctgggcacccagacatatatctgcaacgtgaatcacaagccttccaatacaaaggtggacaagagggtggagccaaagagctgtgataagacccatacatgcccaccttgtccagctccagagctgctgggcggcccatccgtgttcctgtttccacccaagcccaaggacaccctgatgatctctagaaccccagaggtgacatgcgtggtggtggacgtgtcccacgaggatcccgaggtgaagtttaactggtacgtggatggcgtggaggtgcataatgctaagacaaagcccagggaggagcagtacaacagcacctatcgggtggtgtctgtgctgacagtgctgcatcaggactggctgaacggcaaggagtataagtgcaaggtgagcaataaggccctgcctgctccaatcgagaagaccatctctaaggccaagggccagcccagagagcctcaggtgtacacactgcctccaagccgcgaggagatgaccaagaaccaggtgtctctgacatgtctggtgaagggcttctatccctctgacatcgctgtggagtgggagtccaatggccagcctgagaacaattacaagaccacaccccctgtgctggactccgatggcagcttctttctgtattccaagctgaccgtggataagagcaggtggcagcagggcaacgtgttctcctgttctgtgctgcacgaagccctgcactcccattatactcagaagtccctgtccctgtcccctggaaaa

SEQ ID NO: 719 - protein, 449 aa, synthetic construct
  source 1..449: note = MPK190_G1m3-LS VH (aa)
EVQLVESGGGLVKPGGSLTLSCAASGFSISGYGMNWVRQAPGRGLEWVASISASSTYIYYADSMKGRFTISRDNGKTSLYLQMKSLRAEDTAVYYCARAPIEPPYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 720 - DNA, 651 bp, synthetic construct
  source 1..651: mol_type = other DNA; note = MPK190_G1m3-LS VL (nt)
cagtctgtgctgacgcagccgccctcagtgtctggggcccccgggcagagggtcaccatctcctgcacagggagtaactccaacatcggggcaggatatgatgttcactggtaccaacatcttccaggaaaagcccccaaagtcctcatctatggtaacaacaatcggccctcagtggtccctgaccgattctctggctccaactctggcgcctcagcctccctggccatcagtgggctccaggctgaggatgaggctgattatttctgccagtcttatgacaggagactgagtggtccgatcttcggcggagggaccaagctgaccgtcctagggcagcccaaggctgccccctcggtcactctgttcccgccctcgagtgaggagctccaagccaacaaggccaccctggtctgtctgattagcgatttctaccctggtgctgtcactgtcgcatggaaggccgattcatcccccgtcaaagctggtgtcgagacaacaacccccagcaagcagtctaacaataagtacgccgcttccagctatctgtccctgacacctgagcagtggaagagccacaggtcttactcctgtcaggtcactcacgaaggatcaactgtggaaaaaactgtggcaccaaccgaatgttca

SEQ ID NO: 721 - protein, 217 aa, synthetic construct
  source 1..217: note = MPK190__G1m3_LS VL (aa)
QSVLTQPPSVSGAPGQRVTISCTGSNSNIGAGYDVHWYQHLPGKAPKVLIYGNNNRPSVVPDRFSGSNSGASASLAISGLQAEDEADYFCQSYDRRLSGPIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 722 - DNA, 1347 bp, synthetic construct
  source 1..1347: mol_type = other DNA; note = MPK190-v1.3__G1m17,1_LS HC (nt)
caggtgcagctggtggagtctggaggaggactggtgaagccaggaggatctctgaccctgtcctgcgccgctagcggcttcagcatctctggctacggcatgaactgggtgaggcaggctccaggaaggggactggagtgggtggcctccatctccgcctccagcacctacatctactatgctgactccatgaagggcagattcaccatctctcgcgataatggcaagacatccctgtatctgcagatgaagagcctgagggccgaggacacagccgtgtactattgtgccagagctcccatcgagcccccttattttgatctgtggggcaggggcaccctggtgacagtgtcttccgcgtcgaccaagggcccatcggtcttccccctggcaccaagtagcaagagcacatccggtggcacagccgccctgggttgtctggtgaaagattatttccctgagcccgtgacagtctcctggaactctggcgccctgacctccggagtgcacacattccctgctgtgctgcagtccagcggcctgtactccctgtcttccgtggtgaccgtgccaagctcttccctgggcacccagacatatatctgcaacgtgaatcacaagccttccaatacaaaggtggacaagaaggtggagccaaagagctgtgataagacccatacatgcccaccttgtccagctccagagctgctgggcggcccatccgtgttcctgtttccacccaagcccaaggacaccctgatgatctctagaaccccagaggtgacatgcgtggtggtggacgtgtcccacgaggatcccgaggtgaagtttaactggtacgtggatggcgtggaggtgcataatgctaagacaaagcccagggaggagcagtacaacagcacctatcgggtggtgtctgtgctgacagtgctgcatcaggactggctgaacggcaaggagtataagtgcaaggtgagcaataaggccctgcctgctccaatcgagaagaccatctctaaggccaagggccagcccagagagcctcaggtgtacacactgcctccaagccgcgacgagttgaccaagaaccaggtgtctctgacatgtctggtgaagggcttctatccctctgacatcgctgtggagtgggagtccaatggccagcctgagaacaattacaagaccacaccccctgtgctggactccgatggcagcttctttctgtattccaagctgaccgtggataagagcaggtggcagcagggcaacgtgttctcctgttctgtgctgcacgaagccctgcactcccattatactcagaagtccctgtccctgtcccctggaaaa

SEQ ID NO: 723 - protein, 449 aa, synthetic construct
  source 1..449: note = MPK190-v1.3__G1m17,1_LS HC (aa)
QVQLVESGGGLVKPGGSLTLSCAASGFSISGYGMNWVRQAPGRGLEWVASISASSTYIYYADSMKGRFTISRDNGKTSLYLQMKSLRAEDTAVYYCARAPIEPPYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 724 - DNA, 651 bp, synthetic construct
  source 1..651: mol_type = other DNA; note = MPK190-v1.3__G1m17,1_LS LC (nt)
cagtccgtgctgacccagccaccttccgtgagcggagctccaggccagagggtgaccatcagctgcacaggctccagctctaacatcggcgccggctacgacgtgcactggtatcagcatctgccaggcaaggctcccaaggtgctgatctacggcaacacaaatagaccttctgtggtgccagatcgcttctctggctccaatagcggcgcctctgcttccctggctatctccggactgcaggctgaggacgaggctgattacttctgtcagagctatgacaggcggctgtctggccctatctttggcggcggcaccaagctgacagtgctggggcagcccaaggctgccccctcggtcactctgttcccgccctcgagtgaggagctccaagccaacaaggccaccctggtctgtctgattagcgatttctaccctggtgctgtcactgtcgcatggaaggccgattcatcccccgtcaaagctggtgtcgagacaacaacccccagcaagcagtctaacaataagtacgccgcttccagctatctgtccctgacacctgagcagtggaagagccacaggtcttactcctgtcaggtcactcacgaaggatcaactgtggaaaaaactgtggcaccaaccgaatgttca

SEQ ID NO: 725 - protein, 217 aa, synthetic construct
  source 1..217: note = MPK190-v1.3__G1m17,1_LS LC (aa)
QSVLTQPPSVSGAPGQRVTISCTGSSSNIGAGYDVHWYQHLPGKAPKVLIYGNTNRPSVVPDRFSGSNSGASASLAISGLQAEDEADYFCQSYDRRLSGPIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 726 - DNA, 364 bp, synthetic construct
  source 1..364: mol_type = other DNA; note = MPM10 VH (nt)
gaggtgcagctggtggaatctgggggaggcgtggtccagcctgggaggtccctgagactctcctgtgcagcctctggattcattttcagtagctattctatgcactgggtccgccaggctccaggcaaggggctggagtgggcggcagatatttcatttgatggaagcgataaaaactacgcagccgccgtgaatggccgattcaccatctccagagacaattccaagaacacggtgtatctgcaaatgaacggcctgagagctgccgacacggctgtctattattgtgcgagcacttattgcactgatggtacctgctacctcgactactggggccagggaaccctggtcaccgtttcctcag

SEQ ID NO: 727 - protein, 121 aa, synthetic construct
  source 1..121: note = MPM10 VH (aa)
EVQLVESGGGVVQPGRSLRLSCAASGFIFSSYSMHWVRQAPGKGLEWAADISFDGSDKNYAAAVNGRFTISRDNSKNTVYLQMNGLRAADTAVYYCASTYCTDGTCYLDYWGQGTLVTVSS

SEQ ID NO: 728 - protein, 8 aa, synthetic construct
  source 1..8: note = MPM10 CDR-H1 (aa)
GFIFSSYS

SEQ ID NO: 729 - protein, 8 aa, synthetic construct
  source 1..8: note = MPM10 CDR-H2 (aa)
ISFDGSDK

SEQ ID NO: 730 - protein, 14 aa, synthetic construct
  source 1..14: note = MPM10 CDR-H3 (aa)
ASTYCTDGTCYLDY

SEQ ID NO: 731 - DNA, 322 bp, synthetic construct
  source 1..322: mol_type = other DNA; note = MPM10 VK (nt)
cagactgtggtgacccagtctccatcctccctgtctgcttctgtaggggacagcgtcaccatcacttgccgggcaagtcagggcattagaaatgaattaacctggtatcagcaaaaaccagggagagcccctaagcgcctgatctatgctgcgtccagattgcaaagtggggtcccatcaaggttcagcggcagtggatctgggacagaattcgctctcacaataagcagcctgcagcctgaagattttgcaacttattactgtctacagcatagtacttacccgtggacgttcggccaagggaccaaggtggaaatcaaac

SEQ ID NO: 732 - protein, 107 aa, synthetic construct
  source 1..107: note = MPM10 VK (aa)
QTVVTQSPSSLSASVGDSVTITCRASQGIRNELTWYQQKPGRAPKRLIYAASRLQSGVPSRFSGSGSGTEFALTISSLQPEDFATYYCLQHSTYPWTFGQGTKVEIK

SEQ ID NO: 733 - protein, 6 aa, synthetic construct
  source 1..6: note = MPM10 CDR-L1 (aa)
QGIRNE

SEQ ID NO: 734 - skipped

SEQ ID NO: 735 - protein, 9 aa, synthetic construct
  source 1..9: note = MPM10 CDR-L3 (aa)
LQHSTYPWT

SEQ ID NO: 736 - DNA, 361 bp, synthetic construct
  source 1..361: mol_type = other DNA; note = MPM2 VH (nt)
caggtgcagctacagcagtggggcgcaggactgttgaagccttcggagaccctgtccctcacatgcgctgtgtttggtgggtccttcagtgattccttctggagctggatccgccagcccccagggaaggggctggagtggattggagagatcaatcatagtggaatcagtaactacagcccgtccctcaagagtcgtctcaccatatcaatagacacgtccagcagccagttctccctgaggctgacctctacgaccgccgcggacacggctgtttactactgtgcgagagtcgggttcacctatagttccttctactttgactattggggccagggaaccctggtcaccgtctcctcag

SEQ ID NO: 737 - protein, 120 aa, synthetic construct
  source 1..120: note = MPM2 VH (aa)
QVQLQQWGAGLLKPSETLSLTCAVFGGSFSDSFWSWIRQPPGKGLEWIGEINHSGISNYSPSLKSRLTISIDTSSSQFSLRLTSTTAADTAVYYCARVGFTYSSFYFDYWGQGTLVTVSS

SEQ ID NO: 738 - protein, 8 aa, synthetic construct
  source 1..8: note = MPM2; MPM2 VH.2 CDR-H1 (aa)
GGSFSDSF

SEQ ID NO: 739 - protein, 7 aa, synthetic construct
  source 1..7: note = MPM2 CDR-H2 (aa)
INHSGIS

SEQ ID NO: 740 - protein, 14 aa, synthetic construct
  source 1..14: note = MPM2; MPM2 VH.2; MPM2 VH.4; MPM2 VH.5 CDR-H3 (aa)
ARVGFTYSSFYFDY

SEQ ID NO: 741 - DNA, 328 bp, synthetic construct
  source 1..328: mol_type = other DNA; note = MPM2 VL.1; MPM2 VL (nt)
cagactgtggtgactcagccaccctcggtgtcagtggccccaggaaagacggccagcattacctgtgggggagagaacattggaactaaaagtgtgcactggtaccagcagaagccagggcaggcccctgtgttggtcgtccgtgatgatagcgaccggccctcagggatccctgagcgattctctggctccagctctgggaacacggccaccctgaccatcagcagggtcgaagccggggatgaggccgactattactgtcaggtttggcttagtagtggtgatcatccttgggtgttcggcggagggaccaagctgaccgtcctag

SEQ ID NO: 742 - protein, 109 aa, synthetic construct
  source 1..109: note = MPM2 VL.1; MPM2 VL (aa)
QTVVTQPPSVSVAPGKTASITCGGENIGTKSVHWYQQKPGQAPVLVVRDDSDRPSGIPERFSGSSSGNTATLTISRVEAGDEADYYCQVWLSSGDHPWVFGGGTKLTVL

SEQ ID NO: 743 - protein, 6 aa, synthetic construct
  source 1..6: note = MPM2 VL.1; MPM2 VL.2; MPM2 VL.3; MPM2 VL.4; MPM2 VL.5; MPM2 VL.6; MPM2 VL.7; MPM2 VL.8; MPM2 VL.9; MPM2 CDR-L1 (aa)
NIGTKS

SEQ ID NO: 744 - protein, 12 aa, synthetic construct
  source 1..12: note = MPM2 VL.1; MPM2 VL.3; MPM2 VL.4; MPM2 VL.5; MPM2 VL.6; MPM2 VL.7; MPM2 VL.8; MPM2 CDR-L3 (aa)
QVWLSSGDHPWV

SEQ ID NO: 745 - DNA, 364 bp, synthetic construct
  source 1..364: mol_type = other DNA; note = MPM8 VH (nt)
caggtacagctggtgcagtctggcgctgaggtgaagaaacctggggcctctgtgaaggtctcctgcaagacctctggttacatcttaagcaactatggaatgtcctggttccgacaggcccctggacaggggcttgagtggatggcatccgtcagcggttacaatggtaacacaaaatttgcacggaaggtccaggacagagtctccatgaacatagacacatccacgaggacggcctccatggagctgaggggcctaagatctgacgacacggccgtgtattactgtgcgcgagacacgggtttagacgcagcccccctccttgactactggggccagggatccctggtcatcgtctcctcag

SEQ ID NO: 746 - protein, 121 aa, synthetic construct
  source 1..121: note = MPM8 VH (aa)
QVQLVQSGAEVKKPGASVKVSCKTSGYILSNYGMSWFRQAPGQGLEWMASVSGYNGNTKFARKVQDRVSMNIDTSTRTASMELRGLRSDDTAVYYCARDTGLDAAPLLDYWGQGSLVIVSS

SEQ ID NO: 747 - protein, 8 aa, synthetic construct
  source 1..8: note = MPM8 CDR-H1 (aa)
GYILSNYG

SEQ ID NO: 748 - protein, 8 aa, synthetic construct
  source 1..8: note = MPM8 CDR-H2 (aa)
VSGYNGNT

SEQ ID NO: 749 - protein, 14 aa, synthetic construct
  source 1..14: note = MPM8 CDR-H3 (aa)
ARDTGLDAAPLLDY

SEQ ID NO: 750 - DNA, 340 bp, synthetic construct
  source 1..340: mol_type = other DNA; note = MPM8 VK; MPM8 VL (nt)
gatattgtgatgactcagtctccactctccctgtccgtcacccttggacagccggcctccatctcctgcacgtctagtcaaagcctcgtatatagtgatggaaacacctacttgacttggtttcagcagaggccaggccaatctccaaggcgcctaatttataaggtgtctaatcgggactctggggtcccagccagattcagcggcagtgggtcaggcactgctttcacactgagaatcagcagggtggagcctgaggatgttggggtttattactgcatgcaaggtacacacgggccccagtatacctttggccaggggaccaagctggagatcaaac

SEQ ID NO: 751 - protein, 113 aa, synthetic construct
  source 1..113: note = MPM8 VK; MPM8 VL (aa)
DIVMTQSPLSLSVTLGQPASISCTSSQSLVYSDGNTYLTWFQQRPGQSPRRLIYKVSNRDSGVPARFSGSGSGTAFTLRISRVEPEDVGVYYCMQGTHGPQYTFGQGTKLEIK

SEQ ID NO: 752 - skipped

SEQ ID NO: 753 - protein, 10 aa, synthetic construct
  source 1..10: note = MPM8 CDR-L3 (aa)
MQGTHGPQYT

SEQ ID NO: 754 - DNA, 381 bp, synthetic construct
  source 1..381: mol_type = other DNA; note = MPO1 VH (nt)
gaggtgcagctggtggaatctgggggaggcgtggtccagcctgggaggtccctgagactctcctgtgtagccgctggattcaccttcagtaactatggtgtccactgggtccgccaggctccaggcaaggggctggagtgggtggcagctatatcatacgatggaaccagtagatactacgcagactccgtgaagggccgattcaccatctccagagacaatgccaagaacacactgtatctgcaaatgaacagcctaagagctgaggacacggctgtctttttctgtgcgagaccctatgatagtggtgcttattcatggttgggctactactactatatggacgtctggggcgtcgggaccgcggtcaccgtctcctca

SEQ ID NO: 755 - protein, 127 aa, synthetic construct
  source 1..127: note = MPO1 VH (aa)
EVQLVESGGGVVQPGRSLRLSCVAAGFTFSNYGVHWVRQAPGKGLEWVAAISYDGTSRYYADSVKGRFTISRDNAKNTLYLQMNSLRAEDTAVFFCARPYDSGAYSWLGYYYYMDVWGVGTAVTVSS

SEQ ID NO: 756 - protein, 8 aa, synthetic construct
  source 1..8: note = MPO1 CDR-H1 (aa)
GFTFSNYG

SEQ ID NO: 757 - protein, 8 aa, synthetic construct
  source 1..8: note = MPO1 CDR-H2 (aa)
ISYDGTSR

SEQ ID NO: 758 - protein, 20 aa, synthetic construct
  source 1..20: note = MPO1 CDR-H3 (aa)
ARPYDSGAYSWLGYYYYMDV

SEQ ID NO: 759 - DNA, 331 bp, synthetic construct
  source 1..331: mol_type = other DNA; note = MPO1 VL (nt)
cagtctgccctgactcagcctgcctccgtgtctgggtctcctggacagtcgatcaccatctcctgcactggaaccagcagtgacgttggtgcttatacctatgtctcctggtaccaacagcacccaggcaaagcccccaaacttatgatttatgatgtcactaatcggccctcaggggtttctaatcgcttctctggctccaagtctgacaacacggcctccctgaccatctctgggctccaggctgaagacgaggctgattattactgcagctcatatacaagcagcaacactctcgtgttcggcggagggaccaagctgaccgtcctag

SEQ ID NO: 760 - protein, 110 aa, synthetic construct
  source 1..110: note = MPO1 VL (aa)
QSALTQPASVSGSPGQSITISCTGTSSDVGAYTYVSWYQQHPGKAPKLMIYDVTNRPSGVSNRFSGSKSDNTASLTISGLQAEDEADYYCSSYTSSNTLVFGGGTKLTVL

SEQ ID NO: 761 - protein, 9 aa, synthetic construct
  source 1..9: note = MPO1 CDR-L1 (aa)
SSDVGAYTY

SEQ ID NO: 762 - skipped

SEQ ID NO: 763 - protein, 10 aa, synthetic construct
  source 1..10: note = MPO1 CDR-L3 (aa)
SSYTSSNTLV

SEQ ID NO: 764 - DNA, 373 bp, synthetic construct
  source 1..373: mol_type = other DNA; note = MPO7 VH (nt)
gaggtgcagctggtggagtctgggggaggcttgatacagcctggggagtccctgagactctcctgtgcagcctctggattcaactttaacaactatgtcatgagttgggtccgccaggctccagggaaggggctggaatgggtctcaatcattagtgctagtgctgatagaacacactacgcagactccgtgaagggccggttcaccatctccagagacaattccaagaacacgatgtatttgcaaatgaacagcctgagagtcgaggacacggccgtatattactgcgcgaaagatctgttcgaagagtgggagctagttactttctactctgagtcttggggccagggaaccctggtcaccgtctcctcag

SEQ ID NO: 765 - protein, 124 aa, synthetic construct
  source 1..124: note = MPO7 VH (aa)
EVQLVESGGGLIQPGESLRLSCAASGFNFNNYVMSWVRQAPGKGLEWVSIISASADRTHYADSVKGRFTISRDNSKNTMYLQMNSLRVEDTAVYYCAKDLFEEWELVTFYSESWGQGTLVTVSS

SEQ ID NO: 766 - protein, 8 aa, synthetic construct
  source 1..8: note = MPO7 CDR-H1 (aa)
GFNFNNYV

SEQ ID NO: 767 - protein, 8 aa, synthetic construct
  source 1..8: note = MPO7 CDR-H2 (aa)
ISASADRT

SEQ ID NO: 768 - protein, 17 aa, synthetic construct
  source 1..17: note = MPO7 CDR-H3 (aa)
AKDLFEEWELVTFYSES

SEQ ID NO: 769 - DNA, 331 bp, synthetic construct
  source 1..331: mol_type = other DNA; note = MPO7 VL (nt)
cagtctgccctgactcagcctccctccgcgtccgggtctcctggacagtcagtcaccatctcctgcactggaaccagctttgacgttggtggttatacctctgtctcctggtaccaacagcacccagacaaagcccccaaactcttgatttatgaagtctttaagcggccctcaggggtccctgatcgcttctctggctccaagtctggcaacacggcctccctgaccgtctctgggctccaggctgaggatgaggctgattattactgcaactcatattcaggcaccaaccattatgtcttcggaactgggaccaaggtcaccgtcctag

SEQ ID NO: 770 - protein, 109 aa, synthetic construct
  source 1..109: note = MPO7 VL (aa)
QSALTQPPSASGSPGQSVTISCTGTSFDVGGYTSVSWYQQHPDKAPKLLIYEVFKRPSGVPDRFSGSKSGNTASLTVSGLQAEDEADYYCNSYSGTNHYVFGTGTKVTV

SEQ ID NO: 771 - protein, 9 aa, synthetic construct
  source 1..9: note = MPO7 CDR-L1
SFDVGGYTS

SEQ ID NO: 772 - skipped

SEQ ID NO: 773 - protein, 10 aa, synthetic construct
  source 1..10: note = MPO7 CDR-L3
NSYSGTNHYV

SEQ ID NO: 774 - DNA, 379 bp, synthetic construct
  source 1..379: mol_type = other DNA; note = MPP1 VH (nt)
caggtgcagctgcaggagtcgggcccgggactggtgaagccttcggggaccctgtccctcacctgcgttgtctctggtgactccatgaacaatgatgactggtggagttgggtccgccagcccccagggaaggggctggagtggattggagaaatctattctagtgggaccaccaactacaacccgtccgtcaagagtcgagtcagcttatcagtagacaagtccaagaaggagttctccctgaagctgacctctgtgaccgccgcggacacggccgtctattactgtgcgagagggtggggatactgtagcagaaaaagttgctatgccaaagctttcgatatctggggccaagggacactggtcatcgtctcttcag

SEQ ID NO: 775 - protein, 126 aa, synthetic construct
  source 1..126: note = MPP1 VH (aa)
QVQLQESGPGLVKPSGTLSLTCVVSGDSMNNDDWWSWVRQPPGKGLEWIGEIYSSGTTNYNPSVKSRVSLSVDKSKKEFSLKLTSVTAADTAVYYCARGWGYCSRKSCYAKAFDIWGQGTLVIVSS

SEQ ID NO: 776 - protein, 9 aa, synthetic construct
  source 1..9: note = MPP1 CDR-H1 (aa)
GDSMNNDDW

SEQ ID NO: 777 - protein, 7 aa, synthetic construct
  source 1..7: note = MPP1 CDR-H2 (aa)
IYSSGTT

SEQ ID NO: 778 - protein, 19 aa, synthetic construct
  source 1..19: note = MPP1 CDR-H3
ARGWGYCSRKSCYAKAFDI

SEQ ID NO: 779 - DNA, 325 bp, synthetic construct
  source 1..325: mol_type = other DNA; note = MPP1 VK; MPP1 VL (nt)
gacatccagatgacccagtctccatcctccctgtctgcatctgtaggagacagagtcaccatcacttgccaggcgagtcaggacattgacaactatttaaattggtatcagcagaaaccagggaaagcccctaaactcctaatctacgatgcatccactttgaaagaaggggtcccctcaaggttcagtggaagtgggtctgggacagaatttactttcaccatcagcagcctgcagcctgaagatattgcaacatattactgtcaacagtatcataatctccctccgatcaccttcggccaagggacgcgactgcaaattaaac

SEQ ID NO: 780 - protein, 108 aa, synthetic construct
  source 1..108: note = MPP1 VK; MPP1 VL (aa)
DIQMTQSPSSLSASVGDRVTITCQASQDIDNYLNWYQQKPGKAPKLLIYDASTLKEGVPSRFSGSGSGTEFTFTISSLQPEDIATYYCQQYHNLPPITFGQGTRLQIK

SEQ ID NO: 781 - protein, 6 aa, synthetic construct
  source 1..6: note = MPP1 CDR-L1 (aa)
QDIDNY

SEQ ID NO: 782 - protein, 10 aa, synthetic construct
  source 1..10: note = MPP1 CDR-L3 (aa)
QQYHNLPPIT

SEQ ID NO: 783 - DNA, 372 bp, synthetic construct
  source 1..372: mol_type = other DNA; note = MPP2 VH (nt)
caggtgcagctacaggagtccggctcacgactggtgaagccttcacagaccctgtccctcacctgcactgcctctggtatctctatcagcggtaccgattactcctggaactggctccggcagccaccagggaagggcctggagtggattggctacgtctctcaaagtggaattcccaagtacaacccgtccctcaagagtcgagtcaccatatcaattgattggcccagcaatcgcttctccctgaccctgaagtccgtgaccgccgcggacacggccgtgtattactgcgccagagatgtgggagctaccgactactattactacgggatggacgtctggggccaggggaccacggtcatcgtctcctca

SEQ ID NO: 784 - protein, 124 aa, synthetic construct
  source 1..124: note = MPP2 VH (aa)
QVQLQESGSRLVKPSQTLSLTCTASGISISGTDYSWNWLRQPPGKGLEWIGYVSQSGIPKYNPSLKSRVTISIDWPSNRFSLTLKSVTAADTAVYYCARDVGATDYYYYGMDVWGQGTTVIVSS

SEQ ID NO: 785 - protein, 10 aa, synthetic construct
  source 1..10: note = MPP2 CDR-H1 (aa)
GISISGTDYS

SEQ ID NO: 786 - protein, 7 aa, synthetic construct
  source 1..7: note = MPP2 CDR-H2 (aa)
VSQSGIP

SEQ ID NO: 787 - protein, 16 aa, synthetic construct
  source 1..16: note = MPP2 CDR-H3 (aa)
ARDVGATDYYYYGMDV

SEQ ID NO: 788 - DNA, 322 bp, synthetic construct
  source 1..322: mol_type = other DNA; note = MPP2 VK (nt)
gaaattgtgttgacacagtctccaggcaccctgtctttgtctccaggggacagggtcaccctctcctgcagggccagtcagagtgttagccgcaacttcttagcctggtaccaacagaaacctggccaggctcccaggctcctcatttatagtgcatccagcagggtcactggcatcccagacaggttcagtggcagtgggtctgggacagacttcactctcaccatcagcagactggagcctgaagattatgcagtttattactgtcagcagtatgatggctcactcactttcggcggagggaccaaggtggagatcaaac

SEQ ID NO: 789 - protein, 107 aa, synthetic construct
  source 1..107: note = MPP2 VK (aa)
EIVLTQSPGTLSLSPGDRVTLSCRASQSVSRNFLAWYQQKPGQAPRLLIYSASSRVTGIPDRFSGSGSGTDFTLTISRLEPEDYAVYYCQQYDGSLTFGGGTKVEIK

SEQ ID NO: 790 - protein, 7 aa, synthetic construct
  source 1..7: note = MPP2 CDR-L1 (aa)
QSVSRNF

SEQ ID NO: 791 - skipped

SEQ ID NO: 792 - protein, 8 aa, synthetic construct
  source 1..8: note = MPP2 CDR-L3 (aa)
QQYDGSLT

SEQ ID NO: 793 - DNA, 379 bp, synthetic construct
  source 1..379: mol_type = other DNA; note = MPR16 VH (nt)
caggtgcagctggtggagtctgggggacgcgtggtccagcctgggaggtccctgagactctcctgtgtggcgtctggattcaccttcaatgactacggcatgcactgggtccgccaggctccaggcaaggggctggagtgggtggcagttatatcctatgatggaactaataagtactatgcagactccgtgaggggccgattcaccatctccagagacaattccaagaacaccgtacatctgcaaatgaacagcctgcgagccgaggacacggctgtgtattactgtgcgagaggatattgtggtagtaatgcctgttttgcctgggtttcggatttagattggtggggccagggaaccctggtcaccgcctcctcag

SEQ ID NO: 794 - protein, 126 aa, synthetic construct
  source 1..126: note = MPR16 VH (aa)
QVQLVESGGRVVQPGRSLRLSCVASGFTFNDYGMHWVRQAPGKGLEWVAVISYDGTNKYYADSVRGRFTISRDNSKNTVHLQMNSLRAEDTAVYYCARGYCGSNACFAWVSDLDWWGQGTLVTASS

SEQ ID NO: 795 - protein, 8 aa, synthetic construct
  source 1..8: note = MPR16 CDR-H1 (aa)
GFTFNDYG

SEQ ID NO: 796 - protein, 8 aa, synthetic construct
  source 1..8: note = MPR16; MPR19-v2 CDR-H2 (aa)
ISYDGTNK

SEQ ID NO: 797 - protein, 19 aa, synthetic construct
  source 1..19: note = MPR16 CDR-H3 (aa)
ARGYCGSNACFAWVSDLDW

SEQ ID NO: 798 - DNA, 328 bp, synthetic construct
  source 1..328: mol_type = other DNA; note = MPR16 VL (nt)
cagtctgtgctgactcagcctgcctccgtgtctgggtctcctggacagtcgatcaccatctcctgcactggaaccagcagtgatgttgggagctttaaccttgtctcctggtaccaacaatacccaggcaaagcccccaaactcatcatttatgacgtcattgagcggccctcagggatttctaagcgcttctctggctccaagtctggcaacacggcctccctgacaatctctggactccaggctgaggacgaggctgattattactgctgctcagatgcaggtcatatctgggtgttcggcggagggaccaggctgaccgtcctaa

SEQ ID NO: 799 - protein, 109 aa, synthetic construct
  source 1..109: note = MPR16 VL (aa)
QSVLTQPASVSGSPGQSITISCTGTSSDVGSFNLVSWYQQYPGKAPKLIIYDVIERPSGISKRFSGSKSGNTASLTISGLQAEDEADYYCCSDAGHIWVFGGGTRLTVL

SEQ ID NO: 800 - protein, 9 aa, synthetic construct
  source 1..9: note = MPR16 CDR-L1 (aa)
SSDVGSFNL

SEQ ID NO: 801 - skipped

SEQ ID NO: 802 - protein, 9 aa, synthetic construct
  source 1..9: note = MPR16 CDR-L3 (aa)
CSDAGHIWV

SEQ ID NO: 803 - DNA, 379 bp, synthetic construct
  source 1..379: mol_type = other DNA; note = MPR19-v2 VH (nt)
caggtgcagctggtggagtctgggggaggcgtggtccagcctgggaagtccctgagcctctcctgcgcagcgtctggattcaccttcaatcagtatgccatgcactgggtccgccaggctccaggcaaggggctggagtgggtggcagttatatcgtatgatggaactaacaagtattatgcagaatccgtgaggggccgattcaccatctccagagacaattccaaggacacggttcatctgcaaatggacagtctgagagccgaggacacggctatgtattactgtgcgagaggatattgtggtagtaatgactgttatatctgggtttcggatttagattggtggggccagggaaccctggtcaccgcctcgtcag

SEQ ID NO: 804 - protein, 126 aa, synthetic construct
  source 1..126: note = MPR19-v2 VH (aa)
QVQLVESGGGVVQPGKSLSLSCAASGFTFNQYAMHWVRQAPGKGLEWVAVISYDGTNKYYAESVRGRFTISRDNSKDTVHLQMDSLRAEDTAMYYCARGYCGSNDCYIWVSDLDWWGQGTLVTASS

SEQ ID NO: 805 - protein, 8 aa, synthetic construct
  source 1..8: note = MPR19-v2 CDR-H1 (aa)
GFTFNQYA

SEQ ID NO: 806 - protein, 19 aa, synthetic construct
  source 1..19: note = MPR19-v2 CDR-H3 (aa)
ARGYCGSNDCYIWVSDLDW

SEQ ID NO: 807 - DNA, 331 bp, synthetic construct
  source 1..331: mol_type = other DNA; note = MPR19-v2 VL (nt)
cagtctgtgctgactcagcctgcctccgtgtctgggtctcctggacagtcgatcaccatctcctgcactgccaccgccggcggtgatgttggaagttttaaccttgtctcctggttccaacaacacccaggcaaagcccccaaactcatactttatgacgtcattgagcggccctcaggaatttctaagcgcttctctggctccaagtctggcagcacggcctccctgacaatctctggactccaggctgaggacgaggctgactattactgctgctcagatgcaggcaatatctgggtgttcggcggagggaccaggctgaccgtcctgc

SEQ ID NO: 808 - protein, 110 aa, synthetic construct
  source 1..110: note = MPR19-v2 VL (aa)
QSVLTQPASVSGSPGQSITISCTATAGGDVGSFNLVSWFQQHPGKAPKLILYDVIERPSGISKRFSGSKSGSTASLTISGLQAEDEADYYCCSDAGNIWVFGGGTRLTVL

SEQ ID NO: 809 - protein, 10 aa, synthetic construct
  source 1..10: note = MPR19-v2 CDR-L1 (aa)
AGGDVGSFNL

SEQ ID NO: 810 - protein, 9 aa, synthetic construct
  source 1..9: note = MPR19-v2 CDR-L3 (aa)
CSDAGNIWV

SEQ ID NO: 811 - DNA, 390 bp, synthetic construct
  source 1..390: mol_type = other DNA; note = MPK65-v2 VH; MPK65 VH (VH.1) (hamster codon optimized) (nt)
caagtgccactagtgcaaagtggtgcagaagtgaagaagcctggagagtccctgaagatctcctgcaagggctctggctactccttcatcaactactggatcggctgggtgcggcagatgcctggcaagggcctggaatggatgggcgtgatctaccccttcgactccgataccaagtattctccttcttttcaaggccacgtgaccatctctgccgacaagtcctccaacaccgcctacgtgcagtggtccagcctgaaagcttctgatacagctatgtactactgtgctagattcaccttcggcatcgccgtggtgaccgacgacgtgtccggcgagggacaggccttcgagatctggggccagggcaccatggtcaccgtgtctagc

SEQ ID NO: 812 - DNA, 390 bp, synthetic construct
  source 1..390: mol_type = other DNA; note = MPK65 VH (VH.2) N31S-D55Q (nt)
caagtgccactagtgcaaagtggtgcagaagtgaagaagcctggagagtctctgaagatctcctgcaagggctccggctactccttcatcagctactggatcggctgggtgcggcagatgcctggaaaaggcctggaatggatgggcgtgatctaccccttccagtccgacaccaagtattctcctagctttcagggccacgtgaccatctctgctgacaagtcctccaacaccgcctacgtgcagtggtccagcctgaaggcctctgatacagctatgtactactgtgctagattcaccttcggcatcgccgtggtgaccgacgacgtgtctggcgagggccaggccttcgagatctggggccaaggcacaatggtcaccgtgtcctct

SEQ ID NO: 813 - protein, 130 aa, synthetic construct
  source 1..130: note = MPK65 VH (VH.2) N31S-D55Q (aa)
QVPLVQSGAEVKKPGESLKISCKGSGYSFISYWIGWVRQMPGKGLEWMGVIYPFQSDTKYSPSFQGHVTISADKSSNTAYVQWSSLKASDTAMYYCARFTFGIAVVTDDVSGEGQAFEIWGQGTMVTVSS

SEQ ID NO: 814 - protein, 8 aa, synthetic construct
  source 1..8: note = MPK65 VH.2 N31S-D55Q; MPK65 VH.3; MPK65 VH.4; MPK65 VH.5; MPK65 VH.6; MPK201 VH.4; MPK201 VH.5; MPK201 VH.6 CDR-H1 (aa)
GYSFISYW

SEQ ID NO: 815 - protein, 8 aa, synthetic construct
  source 1..8: note = MPK65 VH.2 VH N31S-D55Q; MPK65 VH.6; MPK65 VH.7 CDR-H2 (aa)
IYPFQSDT

SEQ ID NO: 816 - DNA, 390 bp, synthetic construct
  source 1..390: mol_type = other DNA; note = MPK65 VH (VH.3) N31S-S56A (nt)
caagtgccactagtgcaaagtggtgcagaagtgaagaagcctggagagtctctgaagatctcctgcaagggctctggctactccttcatctcctactggatcggctgggtgcggcagatgcctggcaaaggcctggaatggatgggcgtgatctaccccttcgatgctgataccaagtattctccttcttttcaaggccacgtgaccatctctgccgacaagtcctccaacaccgcctacgtgcagtggtccagcctgaaggcctccgacaccgctatgtactactgtgctagattcaccttcggcatcgccgtggtgacagacgacgtgtccggcgagggacaggccttcgagatctggggccagggcaccatggtcaccgtgtctagc

SEQ ID NO: 817 - protein, 130 aa, synthetic construct
  source 1..130: note = MPK65 VH (VH.3) N31S-S56A (aa)
QVPLVQSGAEVKKPGESLKISCKGSGYSFISYWIGWVRQMPGKGLEWMGVIYPFDADTKYSPSFQGHVTISADKSSNTAYVQWSSLKASDTAMYYCARFTFGIAVVTDDVSGEGQAFEIWGQGTMVTVSS

SEQ ID NO: 818 - protein, 8 aa, synthetic construct
  source 1..8: note = MPK65 VH.3 N31S-S56A CDR-H2 (aa)
IYPFDADT

SEQ ID NO: 819 - DNA, 390 bp, synthetic construct
  source 1..390: mol_type = other DNA; note = MPK65 VH (VH.4) N31S-D108S (nt)
caagtgccactagtgcaaagtggtgcagaagtgaagaagcctggagagtccctgaagatctcctgcaagggctctggctactccttcatctcctactggatcggctgggtgcggcagatgcctggcaaaggcctggaatggatgggcgtgatctatcctttcgactccgacaccaagtacagccccagctttcagggccacgtgacaatctctgctgacaagtcctccaacaccgcctacgtgcagtggtcctctctgaaggcctctgatacagctatgtactactgtgctagattcaccttcggcatcgccgtggtgacctctgacgtgtccggcgagggccaggccttcgagatctggggacaaggcaccatggtcaccgtgtctagc

SEQ ID NO: 820 - protein, 130 aa, synthetic construct
  source 1..130: note = MPK65 VH (VH.4) N31S-D108S (aa)
QVPLVQSGAEVKKPGESLKISCKGSGYSFISYWIGWVRQMPGKGLEWMGVIYPFDSDTKYSPSFQGHVTISADKSSNTAYVQWSSLKASDTAMYYCARFTFGIAVVTSDVSGEGQAFEIWGQGTMVTVSS

SEQ ID NO: 821 - protein, 23 aa, synthetic construct
  source 1..23: note = MPK65 VH.4 N31S-D108S CDR-H3 (aa)
ARFTFGIAVVTSDVSGEGQAFEI

SEQ ID NO: 822 - DNA, 390 bp, synthetic construct
  source 1..390: mol_type = other DNA; note = MPK65 VH (VH.5) N31S-D108E (nt)
caagtgccactagtgcaaagtggtgcagaagtgaagaagcctggagaatctctgaagatctcctgcaagggctctggctactccttcatctcctactggatcggctgggtgcggcagatgcctggaaaaggcctggaatggatgggcgtgatctatcccttcgactccgacacaaagtactctcctagctttcagggccacgtgaccatctctgctgacaagtcctccaacaccgcctacgtgcagtggtccagcctgaaggcctctgatacagctatgtactactgtgctagattcaccttcggcatcgccgtggtgaccgaggacgtgtccggcgagggccaggccttcgagatctggggccaaggcaccatggtcaccgtgtctagc

SEQ ID NO: 823 - protein, 130 aa, synthetic construct
  source 1..130: note = MPK65 VH (VH.5) N31S-D108E (aa)
QVPLVQSGAEVKKPGESLKISCKGSGYSFISYWIGWVRQMPGKGLEWMGVIYPFDSDTKYSPSFQGHVTISADKSSNTAYVQWSSLKASDTAMYYCARFTFGIAVVTEDVSGEGQAFEIWGQGTMVTVSS

SEQ ID NO: 824 - protein, 23 aa, synthetic construct
  source 1..23: note = MPK65 VH.5 N31S-D108E; MPK65 VH.7 CDR-H3 (aa)
ARFTFGIAVVTEDVSGEGQAFEI

SEQ ID NO: 825 - DNA, 390 bp, synthetic construct
  source 1..390: mol_type = other DNA; note = MPK65 VH (VH.6) N31S-D55Q-D108S (nt)
caagtgccactagtgcaaagtggtgcagaagtgaagaagcctggagagtccctgaagatctcctgcaagggctctggctatagcttcatctcctactggatcggctgggtgcggcagatgcctggcaaaggcctggaatggatgggcgtgatctaccccttccagtccgacaccaagtactcccctagctttcagggccacgtgacaatctctgctgacaagtcctccaacaccgcctacgtgcagtggtcctctctgaaggcctctgataccgctatgtactactgtgctagattcaccttcggcatcgccgtggtgacctctgacgtgtccggcgagggccaggccttcgagatctggggacaaggcaccatggtcaccgtgtctagc

SEQ ID NO: 826 - protein, 130 aa, synthetic construct
  source 1..130: note = MPK65 VH (VH.6) N31S-D55Q-D108S (aa)
QVPLVQSGAEVKKPGESLKISCKGSGYSFISYWIGWVRQMPGKGLEWMGVIYPFQSDTKYSPSFQGHVTISADKSSNTAYVQWSSLKASDTAMYYCARFTFGIAVVTSDVSGEGQAFEIWGQGTMVTVSS

SEQ ID NO: 827 - DNA, 390 bp, synthetic construct
  source 1..390: mol_type = other DNA; note = MPK65 VH.7 N31S-S56A-D108E (nt)
caagtgccactagtgcaaagtggtgcagaagtgaagaagcctggcgagtccctgaaaatctcctgcaagggatccggatactccttcatctcctactggatcggctgggtgcggcagatgcccggcaagggcctggaatggatgggcgtgatctaccctttccagagcgacacaaagtattctccttcttttcaaggccacgtgacaatctctgctgacaagtccagcaacaccgcctacgtgcagtggtccagcctgaaggcctctgataccgctatgtactactgtgctagattcaccttcggcatcgccgtggtgaccgaggacgtgtctggcgaaggccaggccttcgagatctggggccagggcaccatggtcaccgtgtcctct

SEQ ID NO: 828 - protein, 130 aa, synthetic construct
  source 1..130: note = MPK65 VH.7 N31S-S56A-D108E (aa)
QVPLVQSGAEVKKPGESLKISCKGSGYSFISYWIGWVRQMPGKGLEWMGVIYPFQSDTKYSPSFQGHVTISADKSSNTAYVQWSSLKASDTAMYYCARFTFGIAVVTEDVSGEGQAFEIWGQGTMVTVSS

SEQ ID NO: 829 - DNA, 369 bp, synthetic construct
  source 1..369: mol_type = other DNA; note = MPK176 VH.1 (nt) (hamster codon optimized)
caagtgcaactagtgcaaagtggtgcagaagtgaagaaacctggcgcttccgtgcgggtgtcctgcaaggcctctggctacaccctgaccgcctacggcatctcctgggtcagacaggctccaggacagggcctggagtggatgggctggatctctcccaagcacggcaacacccactacgcccagcggttccagggcagagtgaccatgaccaccgatacctccacctctacagcttacatggaactgagatccctgcggcctgacgacaccgctgtgtacttttgtgccagagagggcgtggacatcatggccgtgccttggtatttcgactactggggccaaggaacactggtggccgtgtctagc

SEQ ID NO: 830 - DNA, 369 bp, synthetic construct
  source 1..369: mol_type = other DNA; note = MPK176 VH.2 M104V (nt)
caagtgcaactagtgcaaagtggtgcagaagtgaagaaacccggcgcttccgtgcgggtgtcctgcaaggcctctggatacaccctgaccgcctacggcatctcttgggtcagacaggcccctggccagggcctggagtggatgggctggatctcccctaagcacggcaacacccactacgcccagcggttccagggaagagtgacaatgaccaccgacacctctacctccaccgcttacatggaactgcggtccctgagaccagacgatacagctgtgtacttttgtgccagagagggcgtggacatcgttgctgtgccttggtatttcgactactggggccaaggcaccctggtggccgtgtctagc

SEQ ID NO: 831 - protein, 123 aa, synthetic construct
  source 1..123: note = MPK176 VH.2 M104V (aa)
QVQLVQSGAEVKKPGASVRVSCKASGYTLTAYGISWVRQAPGQGLEWMGWISPKHGNTHYAQRFQGRVTMTTDTSTSTAYMELRSLRPDDTAVYFCAREGVDIVAVPWYFDYWGQGTLVAVSS

SEQ ID NO: 832 - protein, 16 aa, synthetic construct
  source 1..16: note = MPK176 VH.2 M104V CDR-H3 (aa)
AREGVDIVAVPWYFDY

SEQ ID NO: 833 - DNA, 369 bp, synthetic construct
  source 1..369: mol_type = other DNA; note = MPK176 VH.3 VH; MPK176 VH.3 W108F (nt)
caagtgcaactagtgcaaagtggtgcagaagtgaagaaacctggagcttccgtgcgggtgtcctgcaaggcctctggctacaccctgaccgcctacggcatctcctgggtcagacaggctccaggacaaggcctggagtggatgggctggatctctcctaagcacggcaacacccactacgcccagcggttccagggcagagtgaccatgaccaccgacacctccacctctacagcttatatggaactgagatccctgcggcctgacgatacagctgtgtacttttgtgccagagagggcgtggacatcatggccgtgcccttctacttcgactactggggccagggcaccctggtggccgtgtctagc

SEQ ID NO: 834 - protein, 123 aa, synthetic construct
  source 1..123: note = MPK176 VH.3 VH; MPK176 VH.3 W108F (aa)
QVQLVQSGAEVKKPGASVRVSCKASGYTLTAYGISWVRQAPGQGLEWMGWISPKHGNTHYAQRFQGRVTMTTDTSTSTAYMELRSLRPDDTAVYFCAREGVDIMAVPFYFDYWGQGTLVAVSS

SEQ ID NO: 835 - protein, 16 aa, synthetic construct
  source 1..16: note = MPK176 VH.3; MPK176 VH.3 W108F CRD-H3 (aa)
AREGVDIMAVPFYFDY

SEQ ID NO: 836 - DNA, 369 bp, synthetic construct
  source 1..369: mol_type = other DNA; note = MPK176 VH.4 VH; MPK176 VH.4 N57S (nt)
caagtgcaactagtgcaaagtggtgcagaagtgaagaaacctggagcttccgtgcgggtgtcctgcaaggcctctggctacaccctgaccgcttatggcatctcctgggtcagacaggctccaggacaaggcctggagtggatgggctggatctctcccaagcacggctctacccactacgcccagcggttccagggcagagtgaccatgaccaccgatacaagcacctccacagcctacatggaactgagatccctgcggcctgacgacaccgccgtgtacttttgtgccagagagggcgtggacatcatggccgtgccttggtacttcgactactggggccagggcaccctggtggctgtgtctagc

SEQ ID NO: 837 - protein, 123 aa, synthetic construct
  source 1..123: note = MPK176 VH.4; MPK176 VH.4 N57S (aa)
QVQLVQSGAEVKKPGASVRVSCKASGYTLTAYGISWVRQAPGQGLEWMGWISPKHGSTHYAQRFQGRVTMTTDTSTSTAYMELRSLRPDDTAVYFCAREGVDIMAVPWYFDYWGQGTLVAVSS

SEQ ID NO: 838 - protein, 8 aa, synthetic construct
  source 1..8: note = MPK176 VH.6; MPK176 VH.4; MPK176 VH.4 N57S CDR-H2 (aa)
ISPKHGST

SEQ ID NO: 839 - DNA, 369 bp, synthetic construct
  source 1..369: mol_type = other DNA; note = MPK176 VH.5 VH; MPK176 VH.5 D111A (nt)
caagtgcaactagtgcaaagtggtgcagaagtgaagaaacccggagcttccgtgcgggtgagctgcaaggcctctggctacaccctgaccgcttatggcatctcctgggtcagacaggctcctggccagggcctggagtggatgggctggatctctccaaagcacggcaacacccactacgcccagcggttccagggcagagtgaccatgaccaccgacacctctacctccaccgcctacatggaactgagatccctgcggcctgacgatacagctgtgtacttttgtgccagagagggcgtggacatcatggccgtgccttggtacttcgcctactggggccaaggaacactggtggccgtgtccagc

SEQ ID NO: 840 - protein, 123 aa, synthetic construct
  source 1..123: note = MPK176 VH.5 VH; MPK176 VH.5 D111A (aa)
QVQLVQSGAEVKKPGASVRVSCKASGYTLTAYGISWVRQAPGQGLEWMGWISPKHGNTHYAQRFQGRVTMTTDTSTSTAYMELRSLRPDDTAVYFCAREGVDIMAVPWYFAYWGQGTLVAVSS

SEQ ID NO: 841 - protein, 16 aa, synthetic construct
  source 1..16: note = MPK176 VH.5; MPK176 VH.5 D111A CDR-H3 (aa)
AREGVDIMAVPWYFAY

SEQ ID NO: 842 - DNA, 369 bp, synthetic construct
  source 1..369: mol_type = other DNA; note = MPK176 VH.6 VH; MPK176 VH.6 N57S-M104V-W108F-D111A (nt)
caagtgcaactagtgcaaagtggtgcagaagtgaagaaacctggcgcttccgtgcgggtgtcctgcaaggcctctggctacaccctgaccgcttatggcatctcctgggttagacaggccccaggacaaggcctggagtggatgggctggatctctcctaagcacggctccacccactacgcccagcggttccagggcagagtgaccatgaccaccgatacctctaccagcacagcttacatggaactgagatccctgcggcctgacgacaccgccgtgtacttttgtgccagagagggcgtggacatcgtggccgtccccttctacttcgcctactggggccagggaacactggtggctgtgtctagc

SEQ ID NO: 843 - protein, 123 aa, synthetic construct
  source 1..123: note = MPK176 VH.6 VH; MPK176 VH.6 N57S-M104V-W108F-D111A (aa)
QVQLVQSGAEVKKPGASVRVSCKASGYTLTAYGISWVRQAPGQGLEWMGWISPKHGSTHYAQRFQGRVTMTTDTSTSTAYMELRSLRPDDTAVYFCAREGVDIVAVPFYFAYWGQGTLVAVSS

SEQ ID NO: 844 - protein, 16 aa, synthetic construct
  source 1..16: note = MPK176 VH.6; MPK176 VH.6 N57S-M104V-W108F-D111A CDR-H3 (aa)
AREGVDIVAVPFYFAY

SEQ ID NO: 845 - DNA, 321 bp, synthetic construct
  source 1..321: mol_type = other DNA; note = MPK201 VK.1 (nt) (hamster codon optimized)
gaaatcgtgatgatgcaaagtccagcaactctgtctgtgtcccctggcgagcgggctacactgtcctgcagagcctctcagtccgtggccggcaacctggcctggtaccagcagcggccaggacaagctcctagactcctggtctatggcgcttccaccagagcttctggcgtgcccgccagattctccggaagcggctctgaaaccgagttcaccctgaccatctccagcctgcagtctgaggacttcgccgtgtactactgtcaccactactccgattggcctcaggcctttggccagggcacccggctggacatcaag

SEQ ID NO: 846 - DNA, 321 bp, synthetic construct
  source 1..321: mol_type = other DNA; note = MPK201 VK.2 E1Q-W94L; MPK201 VK.2 VL (nt)
caaatcgtgatgatgcaaagtccagcaactctctctgtgtcccccggcgagcgggctacactgtcctgcagagcctctcagtccgtggccggcaacctggcctggtaccagcagcggcctggccaagctcctagactgctggtctatggcgcttccaccagagcttctggcgtgccagccagattctccggctctggaagcgaaaccgagttcaccctgaccatctccagcctgcagtctgaggacttcgccgtgtactactgtcaccactactccgatctgcctcaggcctttggccagggcacccggctggacatcaag

SEQ ID NO: 847 - protein, 107 aa, synthetic construct
  source 1..107: note = MPK201 VK.2 E1Q-W94L; MPK201 VK.2 VL (aa)
QIVMMQSPATLSVSPGERATLSCRASQSVAGNLAWYQQRPGQAPRLLVYGASTRASGVPARFSGSGSETEFTLTISSLQSEDFAVYYCHHYSDLPQAFGQGTRLDIK

SEQ ID NO: 848 - protein, 9 aa, synthetic construct
  source 1..9: note = MPK201 VK.2 E1Q-W94L; MPK201 VK.2 CDRL3 (aa)
HHYSDLPQA

SEQ ID NO: 849 - DNA, 321 bp, synthetic construct
  source 1..321: mol_type = other DNA; note = MPK65 VK.1 VL (nt) (hamster codon optimized)
gaaatcgtgatgatgcaaagtccagcaactctgtccgtgtcccccggcgagagagctacactgtcttgtagagcctctcaatctgtggccggcaacctggcctggtatcagcagcggcctggccaggctcctcggctgctgatctacggcgcttctacccgggctaccggcgtcccagccagattctccggctctggcagcgagacagaattcaccctgaccatctccagcctgcagtccgaggacttcgccgtgtactactgccaccagtactccaactggcctcaggcctttggacagggcaccagactcgatatcaag

SEQ ID NO: 850 - DNA, 321 bp, synthetic construct
  source 1..321: mol_type = other DNA; note = MPK65 VK.2 VL E1Q-W94L (nt)
caaatcgtgatgatgcaaagtccagcaactctgtccgtgtcccccggcgagcgggccaccctctcttgtagagcctctcagagcgtcgctggcaacctggcctggtatcagcagcggcctggccaagctcctagactgctgatctacggcgcttctaccagagctaccggcgtgccagccagattctccggctctggatctgaaaccgagttcaccctgaccatctccagcctgcagtccgaggacttcgccgtgtactactgccaccagtactccaacctgcctcaggcctttggccagggcacacggctggatatcaag

SEQ ID NO: 851 - protein, 107 aa, synthetic construct
  source 1..107: note = MPK65 VK.2 VL E1Q-W94L (aa)
QIVMMQSPATLSVSPGERATLSCRASQSVAGNLAWYQQRPGQAPRLLIYGASTRATGVPARFSGSGSETEFTLTISSLQSEDFAVYYCHQYSNLPQAFGQGTRLDIK

SEQ ID NO: 852 - protein, 9 aa, synthetic construct
  source 1..9: note = MPK65 VK.2 E1Q-W94L CDR-L3 (aa)
HQYSNLPQA

SEQ ID NO: 853 - DNA, 324 bp, synthetic construct
  source 1..324: mol_type = other DNA; note = MPK176 VL.1 (nt) (hamster codon optimized)
agttatgaactaactcaaccaccaagtgtgtctgtggctcctggccagaccgccagaatcagctgtggcggcaacaacatcggctctaagtccgtgcactggtaccagcagaaaccaggacaggcccctgtggtggtcgtgtacgacgactccgatagaccttctggcatccccgagcggtttagcggctccaactccggcaataccgctaccctgaccatctccggcgtggaagctggcgacgaggccgactacttctgccaagtgtgggattctgccaccgaccactgggtgttcggcggaggcaccaagctgacagtgctg

SEQ ID NO: 854 - DNA, 324 bp, synthetic construct
  source 1..324: mol_type = other DNA; note = MPK176 VL.2 E3V-D49Q; MPK176 VL.2 VL (nt)
agttatgtgctaactcaaccaccaagtgtgtctgtggcccctggacaaacagccagaatctcctgtggcggcaacaacatcggctctaagtccgtgcactggtaccagcagaaacctggccaggctcctgtggtggtcgtgtaccaggactccgatagaccatctggcatccccgagcggtttagcggctccaactccggcaataccgctaccctgaccatctctggcgtggaagctggcgacgaggccgactacttctgccaggtgtgggatagcgccaccgaccactgggtgttcggcggaggcaccaagctgaccgtgctg

SEQ ID NO: 855 - protein, 108 aa, synthetic construct
  source 1..108: note = MPK176 VL.2 E3V-D49Q; MPK176 VL.2 VL (aa)
SYVLTQPPSVSVAPGQTARISCGGNNIGSKSVHWYQQKPGQAPVVVVYQDSDRPSGIPERFSGSNSGNTATLTISGVEAGDEADYFCQVWDSATDHWVFGGGTKLTVL

SEQ ID NO: 856 - skipped

SEQ ID NO: 857 - DNA, 324 bp, synthetic construct
  source 1..324: mol_type = other DNA; note = MPK176 VL.3 E3V-D52K; MPK176 VL.3 VL (nt)
agttatgtgctaactcaaccaccaagtgtgtctgtggcccccggacagaccgccagaatcagctgtggcggcaacaacatcggctctaagtccgtgcactggtaccagcagaaacctggccaggcccctgtggtggtcgtgtacgacgactccaagcggccatctggcatccctgagagatttagcggctccaactccggcaataccgctacactgaccatctccggcgtggaagctggcgacgaggccgactacttctgccaagtgtgggattctgctaccgatcactgggtgttcggcggaggcaccaagctgaccgtgctg

SEQ ID NO: 858 - protein, 108 aa, synthetic construct
  source 1..108: note = MPK176 VL.3 E3V-D52K; MPK176 VL.3 VL (aa)
SYVLTQPPSVSVAPGQTARISCGGNNIGSKSVHWYQQKPGQAPVVVVYDDSKRPSGIPERFSGSNSGNTATLTISGVEAGDEADYFCQVWDSATDHWVFGGGTKLTVL

SEQ ID NO: 859 - DNA, 324 bp, synthetic construct
  source 1..324: mol_type = other DNA; note = MPK176 VL.4 E3V-N25A; MPK176 VL.4 VL (nt)
agttatgtgctaactcaaccaccaagtgtgtctgtggcccctggacagaccgccagaatcagctgtggcggcgccaacatcggctctaagtccgtgcactggtaccagcagaaacctggccaggctcctgtggtggtcgtgtacgacgactccgatagaccatctggcatccccgagcggtttagcggctccaactccggcaacaccgctaccctgacaatctccggcgtggaagctggcgacgaggccgactacttctgccaagtgtgggattctgccaccgaccactgggtgttcggcggaggcaccaagctgaccgtgctg

SEQ ID NO: 860 - protein, 108 aa, synthetic construct
  source 1..108: note = MPK176 VL.4 E3V-N25A; MPK176 VL.4 VL (aa)
SYVLTQPPSVSVAPGQTARISCGGANIGSKSVHWYQQKPGQAPVVVVYDDSDRPSGIPERFSGSNSGNTATLTISGVEAGDEADYFCQVWDSATDHWVFGGGTKLTVL

SEQ ID NO: 861 - DNA, 324 bp, synthetic construct
  source 1..324: mol_type = other DNA; note = MPK176 VL.5 E3V-D49Q-D52K-N25A; MPK176 VL.5 VL (nt)
agttatgtgctaactcaaccaccaagtgtgtctgtggcccctggccagaccgcccggatctcctgtggcggagccaacatcggctctaagtccgtgcactggtaccagcagaagcctggacaagctcctgttgtggtcgtgtaccaggattccaaaagaccatctggcatccccgagagatttagcggctccaactccggcaacaccgctaccctgacaatcagcggcgtggaagctggcgacgaggccgactacttctgccaggtgtgggactctgccaccgaccactgggtgttcggcggcggcaccaagctgaccgtgctg

SEQ ID NO: 862 - protein, 108 aa, synthetic construct
  source 1..108: note = MPK176 VL.5 E3V-D49Q-D52K-N25A; MPK176 VL.5 VL (aa)
SYVLTQPPSVSVAPGQTARISCGGANIGSKSVHWYQQKPGQAPVVVVYQDSKRPSGIPERFSGSNSGNTATLTISGVEAGDEADYFCQVWDSATDHWVFGGGTKLTVL

SEQ ID NO: 863 - DNA, 327 bp, synthetic construct
  source 1..327: mol_type = other DNA; note = MPM2 VL.1 (nt) (hamster codon optimized)
caaactgtggtgactcaaccaccaagtgtgtctgtggcccctggcaagaccgcttccatcacctgtggcggcgagaacatcggcaccaagtccgtgcactggtaccagcagaaacccggccaggctcctgtgctggtggtgcgggacgactccgatagaccatctggcatccctgagcggttctccggctctagctctggcaacaccgccaccctgaccatctccagagtggaagccggcgacgaggccgactactactgccaagtgttcctgtccagcggcgatcacccttgggtctttggcggaggaacaaagctgaccgtgctg

SEQ ID NO: 864 - DNA, 327 bp, synthetic construct; identical to SEQ ID NO: 863
  source 1..327: mol_type = other DNA; note = MPM2 VL.2 W90F; MPM2 VL.2 VL (nt)

SEQ ID NO: 865 - protein, 109 aa, synthetic construct
  source 1..109: note = MPM2 VL.2 W90F; MPM2 VL.2 VL (aa)
QTVVTQPPSVSVAPGKTASITCGGENIGTKSVHWYQQKPGQAPVLVVRDDSDRPSGIPERFSGSSSGNTATLTISRVEAGDEADYYCQVFLSSGDHPWVFGGGTKLTVL

SEQ ID NO: 866 - protein, 12 aa, synthetic construct
  source 1..12: note = MPM2 VL.2 W90F; MPM2 VL.2; MPM2 VL.9 CDR-H3 (aa)
QVFLSSGDHPWV

SEQ ID NO: 867 - DNA, 327 bp, synthetic construct
  source 1..327: mol_type = other DNA; note = MPM2 VL.3 D49Q; MPM2 VL.3 VL (nt)
caaactgtggtgactcaaccaccaagtgtgtctgtggcccctggcaagaccgcctccatcacctgtggaggcgagaacatcggcacaaaaagcgtgcactggtaccagcagaagcccggccaggccccagtgctggtcgtgcggcaggactccgaccggccttctggcatccctgagagattctccggctctagctctggcaacaccgctaccctgaccatctccagagtggaagctggcgacgaggccgactactactgccaagtgtggctgtcctccggcgatcacccttgggtgttcggcggaggcaccaagctgacagtgctg

SEQ ID NO: 868 - protein, 109 aa, synthetic construct
  source 1..109: note = MPM2 VL.3 D49Q; MPM2 VL.3 VL (aa)
QTVVTQPPSVSVAPGKTASITCGGENIGTKSVHWYQQKPGQAPVLVVRQDSDRPSGIPERFSGSSSGNTATLTISRVEAGDEADYYCQVWLSSGDHPWVFGGGTKLTVL

SEQ ID NO: 869 - DNA, 327 bp, synthetic construct
  source 1..327: mol_type = other DNA; note = MPM2 VL.4 D49K; MPM2 VL.4 VL (nt)
caaactgtggtgactcaaccaccaagtgtgtctgtggccccaggcaagaccgcctccatcacctgtggcggcgagaacatcggcaccaagtccgtgcactggtaccagcagaaacctggccaggcccctgtgctggtcgtgcggaaggactccgacagaccttctggcatccccgagcggttctccggctctagctctggcaacaccgctaccctgaccatctccagagtggaagctggcgacgaggccgactactactgccaagtgtggctgtccagcggcgatcacccttgggtgttcggcggaggaacaaagctgacagtgctg

SEQ ID NO: 870 - protein, 109 aa, synthetic construct
  source 1..109: note = MPM2 VL.4 D49K; MPM2 VL.4 VL (aa)
QTVVTQPPSVSVAPGKTASITCGGENIGTKSVHWYQQKPGQAPVLVVRKDSDRPSGIPERFSGSSSGNTATLTISRVEAGDEADYYCQVWLSSGDHPWVFGGGTKLTVL

SEQ ID NO: 871 - skipped

SEQ ID NO: 872 - DNA, 327 bp, synthetic construct
  source 1..327: mol_type = other DNA; note = MPM2 VL.5 D50A; MPM2 VL.5 VL (nt)
caaactgtggtgactcaaccaccaagtgtgtctgtggcccctggcaagaccgcttccatcacctgtggcggcgagaacatcggcaccaagtccgtgcactggtaccagcagaaaccaggccaggctcctgtgctggtcgtgcgggacgcctccgacagaccttctggcatccccgagcggttctccggctctagcagcggcaacaccgccaccctgaccatctccagagtggaagctggcgacgaggccgactactactgccaagtgtggctgtcctctggcgatcacccttgggtgttcggcggaggaacaaagctgaccgtgctg

SEQ ID NO: 873 - protein, 109 aa, synthetic construct
  source 1..109: note = MPM2 VL.5 D50A; MPM2 VL.5 VL (aa)
QTVVTQPPSVSVAPGKTASITCGGENIGTKSVHWYQQKPGQAPVLVVRDASDRPSGIPERFSGSSSGNTATLTISRVEAGDEADYYCQVWLSSGDHPWVFGGGTKLTVL

SEQ ID NO: 874 - DNA, 327 bp, synthetic construct
  source 1..327: mol_type = other DNA; note = MPM2 VL.6 D52Q (nt)
caaactgtggtgactcaaccaccaagtgtgtctgtggcccccggcaagaccgcctccatcacctgtggaggcgagaacatcggcaccaagtccgtgcactggtaccagcagaaacctggccaggcccctgtgctggtcgtgcgggacgactcccaacggccatctggcatccctgagagattctccggctctagcagcggcaacaccgctacactgaccatctccagagtggaagctggcgacgaggccgactactactgccaggtgtggctgtcctctggcgatcacccttgggtgttcggcggcggaacaaagctgaccgtgctg

SEQ ID NO: 875 - protein, 109 aa, synthetic construct
  source 1..109: note = MPM2 VL.6 D52Q; MPM2 VL.6 VL (aa)
QTVVTQPPSVSVAPGKTASITCGGENIGTKSVHWYQQKPGQAPVLVVRDDSQRPSGIPERFSGSSSGNTATLTISRVEAGDEADYYCQVWLSSGDHPWVFGGGTKLTVL

SEQ ID NO: 876 - DNA, 327 bp, synthetic construct
  source 1..327: mol_type = other DNA; note = MPM2 VL.7 W90F-D49Q-D52Q; MPM2 VL.7 VL (nt)
caaactgtggtgactcaaccaccaagtgtgtctgtggccccaggcaagaccgcttccatcacctgtggcggcgagaacatcggcacaaaatccgtgcactggtaccagcagaagcctggccaggcccctgtgctggtggtgcggcaggactctcagcggccttctggcatccccgagagattctccggctccagctctggcaacaccgccacactgaccatctccagagtggaagctggcgacgaggccgactactactgccaagtgttcctgtccagcggagatcacccttgggtctttggcggaggcaccaagctgaccgtgctg

SEQ ID NO: 877 - protein, 109 aa, synthetic construct
  source 1..109: note = MPM2 VL.7 W90F-D49Q-D52Q; MPM2 VL.7 VL (aa)
QTVVTQPPSVSVAPGKTASITCGGENIGTKSVHWYQQKPGQAPVLVVRQDSQRPSGIPERFSGSSSGNTATLTISRVEAGDEADYYCQVFLSSGDHPWVFGGGTKLTVL

SEQ ID NO: 878 - DNA, 327 bp, synthetic construct
  source 1..327: mol_type = other DNA; note = MPM2 VL.8 W90F-D49K-D52Q; MPM2 VL.8 VL (nt)
caaactgtggtgactcaaccaccaagtgtgtctgtggccccaggcaagaccgcctccatcacctgtggcggcgagaacatcggcaccaagtccgtgcactggtaccagcagaaacctggccaggcccctgtgctggtggtgcggaaggactcccaacggccttctggcatccccgagagattctccggctctagcagcggcaacaccgctaccctgaccatctccagagtggaagctggcgacgaggccgactactactgccaggtgttcctgtcctctggcgatcacccttgggtctttggcggaggaacaaagctgaccgtgctg

SEQ ID NO: 879 - protein, 109 aa, synthetic construct
  source 1..109: note = MPM2 VL.8 W90F-D49K-D52Q; MPM2 VL.8 VL (aa)
QTVVTQPPSVSVAPGKTASITCGGENIGTKSVHWYQQKPGQAPVLVVRKDSQRPSGIPERFSGSSSGNTATLTISRVEAGDEADYYCQVFLSSGDHPWVFGGGTKLTVL

SEQ ID NO: 880 - DNA, 327 bp, synthetic construct
  source 1..327: mol_type = other DNA; note = MPM2 VL.9 W90F-D50A-D52Q; MPM2 VL.9 VL (nt)
caaactgtggtgactcaaccaccaagtgtgagcgtcgctcctggcaagaccgcctccatcacctgtggcggcgagaacatcggcaccaagtccgtgcactggtaccagcagaaacccggccaggcccctgtgctggtggtgcgggacgcctctcagagaccttctggcatcccagagcggttcagcggctcttcctccggcaacaccgctacactgaccatctccagagtggaagctggcgacgaggccgattactactgccaagtgtttctgtcctctggcgaccacccttgggtgttcggcggaggaacaaagctgaccgtgctg

SEQ ID NO: 881 - protein, 109 aa, synthetic construct
  source 1..109: note = MPM2 VL.9 W90F-D50A-D52Q; MPM2 VL.9 VL (nt)
QTVVTQPPSVSVAPGKTASITCGGENIGTKSVHWYQQKPGQAPVLVVRDASQRPSGIPERFSGSSSGNTATLTISRVEAGDEADYYCQVFLSSGDHPWVFGGGTKLTVL

SEQ ID NO: 882 - DNA, 360 bp, synthetic construct
  source 1..360: mol_type = other DNA; note = MPM2 VL.2 N52H-N58Y; MPM2 VH.2 VH (nt)
caagtgcaactacaacaatggggtgcaggtctgctgaaaccctccgagacactgtctctgacctgtgctgtgttcggcggatctttttccgactccttctggtcctggatccggcagcctcctggcaagggcctggaatggatcggagagatccaccactctggcatctcttactattctcctagcctgaagtcccggctcacaatctccatcgacacctcctcttctcagttctccctgagactgacctctaccaccgctgccgataccgccgtgtactactgcgccagagtgggcttcacctactccagcttctacttcgactactggggccagggcaccctggtcaccgtgtccagc

SEQ ID NO: 883 - protein, 120 aa, synthetic construct
  source 1..120: note = MPM2 VL.2 N52H-N58Y; MPM2 VH.2 VH (aa)
QVQLQQWGAGLLKPSETLSLTCAVFGGSFSDSFWSWIRQPPGKGLEWIGEIHHSGISYYSPSLKSRLTISIDTSSSQFSLRLTSTTAADTAVYYCARVGFTYSSFYFDYWGQGTLVTVSS

SEQ ID NO: 884 - protein, 7 aa, synthetic construct
  source 1..7: note = MPM2 VL.2 N52H-N58Y; MPM2 VH.2; MPM2 VH.4; MPM2 VH.5 CDR-H2 (aa)
IHHSGIS

SEQ ID NO: 885 - DNA, 360 bp, synthetic construct
  source 1..360: mol_type = other DNA; note = MPM2 VL.4 N52H-N58Y-D31Q; MPM2 VH.4 VH (nt)
caagtgcaactacaacaatggggtgcaggtctgctgaagccctccgagacactgtccctgacctgtgctgtgttcggcggctccttttctcaatccttctggtcctggatccggcagcctcctggaaagggactggaatggatcggcgagatccaccactctggcatctcctactattctccttctctcaaatcccggctgaccatctccatcgacacctcttctagccagttcagcctgagactgacatctaccaccgctgccgataccgccgtgtactactgcgccagagtgggcttcacctactccagcttctacttcgactactggggccagggcaccctggtcaccgtgtcctct

SEQ ID NO: 886 - protein, 120 aa, synthetic construct
  source 1..120: note = MPM2 VL.4 N52H-N58Y-D31Q; MPM2 VH.4 VH (aa)
QVQLQQWGAGLLKPSETLSLTCAVFGGSFSQSFWSWIRQPPGKGLEWIGEIHHSGISYYSPSLKSRLTISIDTSSSQFSLRLTSTTAADTAVYYCARVGFTYSSFYFDYWGQGTLVTVSS

SEQ ID NO: 887 - protein, 8 aa, synthetic construct
  source 1..8: note = MPM2 VL.4 N52H-N58Y-D31Q; MPM2 VH.4 CDR-H1 (aa)
GGSFSQSF

SEQ ID NO: 888 - DNA, 360 bp, synthetic construct
  source 1..360: mol_type = other DNA; note = MPM2 VL.5 N52H-N58Y-D32A; MPM2 VH.5 VH (nt)
caagtgcaactacaacaatggggtgcaggtctgctgaaaccctccgagacactgagcctcacatgtgccgtgttcggcggatctttttccgacgccttctggtcctggatccggcagcctcctggcaagggcctggaatggatcggcgagatccaccactctggcatcagctactattctccttctctgaagtcccggctgaccatctccatcgacacctcctcttctcagttctccctgagactgacctctaccaccgccgctgataccgctgtgtactactgcgccagagtgggcttcacctactcctccttctacttcgactactggggccagggcaccctggtcaccgtgtccagc

SEQ ID NO: 889 - protein, 120 aa, synthetic construct
  source 1..120: note = MPM2 VL.5 N52H-N58Y-D32A; MPM2 VH.5 VH (aa)
QVQLQQWGAGLLKPSETLSLTCAVFGGSFSDAFWSWIRQPPGKGLEWIGEIHHSGISYYSPSLKSRLTISIDTSSSQFSLRLTSTTAADTAVYYCARVGFTYSSFYFDYWGQGTLVTVSS

SEQ ID NO: 890 - protein, 8 aa, synthetic construct
  source 1..8: note = MPM2 VL.5 N52H-N58Y-D32A; MPM2 VH.5 CDR-H1 (aa)
GGSFSDAF

SEQ ID NO: 891 - DNA, 390 bp, synthetic construct
  source 1..390: mol_type = other DNA; note = MPK201 VH.1 (nt) (hamster codon optimized)
gaagtgcaactagtgcaaagtggtgcagaagtgaagaagcccggcgagtccctgaagatcagctgtcagggatctggctactccttcatcaactactggatcggctgggtgcggcagaaacctggcaagggcctggaatggatgggcatcatctaccctttcgactccgagacaaagtactctcctagcttccagggccacgtgaccttctccgccgacaagtccatcaacaccgcttatgtgcagtggtcctctctgagagcctctgacaccgccatgtactactgcgccagattcacctttggactggctgtcacaaccgctgatgtgtctggcgaaggccaggccttcgagatctggggccaaggcaccatggtgaccgtgtccagc

SEQ ID NO: 892 - DNA, 390 bp, synthetic construct
  source 1..390: mol_type = other DNA; note = MPK201 VH.2 E1Q D55Q; MPK201 VH.2 VH (nt)
caagtgcaactagtgcaaagtggtgcagaagtgaagaagcctggcgagtctctgaagatctcctgccagggatctggctactccttcatcaactactggatcggctgggtgcggcagaaacccggcaagggcctggaatggatgggcatcatctatcctttccagtccgagaccaagtactctcctagctttcagggccacgtgaccttctccgccgacaagtccatcaacaccgcctacgtgcagtggagcagcctgagagcctccgacaccgctatgtactactgtgctagattcaccttcggcctggccgtgacaacagctgatgtgtctggagaaggccaggccttcgagatctggggccaaggcaccatggtcaccgtgtcctct

SEQ ID NO: 893 - protein, 130 aa, synthetic construct
  source 1..130: note = MPK201 VH.2 E1Q D55Q; MPK201 VH.2 VH (aa)
QVQLVQSGAEVKKPGESLKISCQGSGYSFINYWIGWVRQKPGKGLEWMGIIYPFQSETKYSPSFQGHVTFSADKSINTAYVQWSSLRASDTAMYYCARFTFGLAVTTADVSGEGQAFEIWGQGTMVTVSS

SEQ ID NO: 894 - protein, 8 aa, synthetic construct
  source 1..8: note = MPK201 VH.2 E1Q D55Q; MPK201 VH.2; MPK201 VH.5 CDR-H2 (aa)
IYPFQSET

SEQ ID NO: 895 - DNA, 390 bp, synthetic construct
  source 1..390: mol_type = other DNA; note = MPK201 VH.3 E1Q D55S; MPK201 VH.3 VH (nt)
caagtgcaactagtgcaaagtggtgcagaagtgaagaagcctggcgagtccctgaagatctcctgccagggatctggatatagcttcatcaactactggatcggctgggtgcggcagaaacccggcaagggcctggaatggatgggcatcatctaccctttctcctccgagaccaagtactctccttctttccagggccacgtgacattttctgctgataagtccatcaacacagcctacgtgcagtggtcctctctgagagcctccgacaccgctatgtactactgtgctagattcaccttcggcctggccgtgaccaccgccgacgtgtctggcgaaggccaggccttcgagatctggggccaaggcaccatggtcaccgtgtccagc

SEQ ID NO: 896 - protein, 130 aa, synthetic construct
  source 1..130: note = MPK201 VH.3 E1Q D55S; MPK201 VH.3 VH (aa)
QVQLVQSGAEVKKPGESLKISCQGSGYSFINYWIGWVRQKPGKGLEWMGIIYPFSSETKYSPSFQGHVTFSADKSINTAYVQWSSLRASDTAMYYCARFTFGLAVTTADVSGEGQAFEIWGQGTMVTVSS

SEQ ID NO: 897 - protein, 8 aa, synthetic construct
  source 1..8: note = MPK201 VH.3 E1Q D55S; MPK201 VH.3; MPK201 VH.6 CDR-H2 (aa)
IYPFSSET

SEQ ID NO: 898 - DNA, 390 bp, synthetic construct
  source 1..390: mol_type = other DNA; note = MPK201 VH.4 E1Q N31S; MPK201 VH.4- VH (nt)
caagtgcaactagtgcaaagtggtgcagaagtgaagaagcccggcgagtctctgaagatctcttgtcagggctctggctactccttcatctcctactggatcggctgggtgcggcagaagcctggcaaaggactggaatggatgggcatcatctatcctttcgactccgagacaaagtactctccttctttccagggccacgtgaccttctccgccgacaagtccatcaacaccgcctacgtgcagtggtccagcctgagagcttccgacaccgctatgtactactgcgccagattcacctttggcctggccgtgacaaccgctgatgtgtccggcgaaggccaggccttcgagatctggggacaaggcaccatggtcaccgtgtctagc

SEQ ID NO: 899 - protein, 130 aa, synthetic construct
  source 1..130: note = MPK201 VH.4 E1Q N31S; MPK201 VH.4 VH (aa)
QVQLVQSGAEVKKPGESLKISCQGSGYSFISYWIGWVRQKPGKGLEWMGIIYPFDSETKYSPSFQGHVTFSADKSINTAYVQWSSLRASDTAMYYCARFTFGLAVTTADVSGEGQAFEIWGQGTMVTVSS

SEQ ID NO: 900 - DNA, 390 bp, synthetic construct
  source 1..390: mol_type = other DNA; note = MPK201 VH.5 E1Q N31S-D55Q; MPK201 VH.5 VH (nt)
caagtgcaactagtgcaaagtggtgcagaagtgaagaagcctggcgagtccctgaagatctcttgccagggatctggctactccttcatctcctactggatcggctgggtgcggcagaaacccggaaagggcctggaatggatgggcatcatctatcctttccagtccgaaaccaagtacagcccttcttttcagggccacgtgaccttctccgccgacaagtccatcaacaccgcctacgtgcagtggtccagcctgagagcttctgatacagccatgtactactgtgctagattcaccttcggcctggccgtgacaaccgctgacgtgtccggcgagggccaggccttcgagatctggggccaaggcaccatggtcaccgtgtcttct

SEQ ID NO: 901 - protein, 130 aa, synthetic construct
  source 1..130: note = MPK201 VH.5 E1Q N31S-D55Q; MPK201 VH.5 VH (aa)
QVQLVQSGAEVKKPGESLKISCQGSGYSFISYWIGWVRQKPGKGLEWMGIIYPFQSETKYSPSFQGHVTFSADKSINTAYVQWSSLRASDTAMYYCARFTFGLAVTTADVSGEGQAFEIWGQGTMVTVSS

SEQ ID NO: 902 - DNA, 390 bp, synthetic construct
  source 1..390: mol_type = other DNA; note = MPK201 VH.6 E1Q N31S-D55S; MPK201 VH.6 VH (nt)
caagtgcaactagtgcaaagtggtgcagaagtgaagaagcccggagagtccctgaagatcagctgtcagggctctggctattccttcatctcctactggatcggctgggtgcggcagaagcctggcaaaggactggaatggatgggcatcatctaccctttctcctccgagaccaagtactctcctagcttccagggccacgtgaccttttctgctgacaagtccatcaacacagcctacgtgcagtggtcctctctgagagctagcgacaccgccatgtactactgcgccagattcaccttcggcctggccgtgaccacagctgatgtgtccggcgaaggccaggccttcgagatctggggccaaggcaccatggtcaccgtgtcttct

SEQ ID NO: 903 - protein, 130 aa, synthetic construct
  source 1..130: note = MPK201 VH.6 E1Q N31S-D55S; MPK201 VH.6 VH (aa)
QVQLVQSGAEVKKPGESLKISCQGSGYSFISYWIGWVRQKPGKGLEWMGIIYPFSSETKYSPSFQGHVTFSADKSINTAYVQWSSLRASDTAMYYCARFTFGLAVTTADVSGEGQAFEIWGQGTMVTVSS

SEQ ID NO: 904 - DNA, 651 bp, synthetic construct
  source 1..651: mol_type = other DNA; note = MPK190-v1.3-rIgG1m17,1-LS LC (nt) - version 2
cagtccgtgctgacccagccaccttccgtgagcggagctccaggccagagggtgaccatcagctgcacaggctccagctctaacatcggcgccggctacgacgtgcactggtatcagcatctgccaggcaaggctcccaaggtgctgatctacggcaacacaaatagaccttctgtggtgccagatcgcttctctggctccaatagcggcgcctctgcttccctggctatctccggactgcaggctgaggacgaggctgattacttctgtcagagctatgacaggcggctgtctggccctatctttggcggcggcaccaagctgacagtgctgggtcagcccaaggctgccccctcggtcactctgttcccgccctcgagtgaggagctccaagccaacaaggccaccctggtctgtctgattagcgatttctaccctggtgctgtcactgtcgcatggaaggccgattcatcccccgtcaaagctggtgtcgagacaacaacccccagcaagcagtctaacaataagtacgccgcttccagctatctgtccctgacacctgagcagtggaagagccacaggtcttactcctgtcaggtcactcacgaaggatcaactgtggaaaaaactgtggcaccaaccgaatgttca

SEQ ID NO: 905 - protein, 107 aa, synthetic construct [sequence omitted]